(19) United States
(12) Patent Application Publication
(10) Pub. No.: US 2026/0138951 A1
(43) Pub. Date: May 21, 2026

(54) TREATING PKU WITH SPIRO-SUBSTITUTED AND OTHER PIPERIDINE INHIBITORS OF SLC6A19 FUNCTION

(71) Applicant: Jnana Therapeutics Inc., Boston, MA (US)

(72) Inventors: Ryan A. Hollibaugh, Quincy, MA (US); Dean G. Brown, Hollis, NH (US); Joshua E. Zweig, Somerville, MA (US); Giovanni Muncipinto, Cambridge, MA (US); Hyejin Park, Boston, MA (US)

(21) Appl. No.: 19/111,603

(22) PCT Filed: Sep. 14, 2023
(86) PCT No.: PCT/US23/32752

§ 371 Date: Mar. 13, 2025

Related U.S. Application Data
(60) Provisional application No. 63/406,536, filed on Sep. 14, 2022.

(51) Int. Cl. C07D 211/56; A61K 31/438; A61K 31/445; A61K 31/4523; A61K 31/454; A61K 31/4545; A61K 31/501; A61K 31/506; A61K 31/513; C07D 211/60; C07D 221/20; C07D 401/04; C07D 401/06; C07D 413/04; C07D 471/10; C07D 491/107; C07D 491/113; C07D 498/10

(52) U.S. Cl. CPC C07D 211/56; A61K 31/438; A61K 31/445; A61K 31/4523; A61K 31/454; A61K 31/4545; A61K 31/501; A61K 31/506; A61K 31/513; C07D 211/60; C07D 221/20; C07D 401/04; C07D 401/06; C07D 413/04; C07D 471/10; C07D 491/107; C07D 491/113; C07D 498/10

(57) ABSTRACT

Disclosed are compounds, compositions, and methods useful for treating or preventing a disease or disorder associated with abnormal levels of amino acids by modulation of SLC6A 19 transport.

### RELATED APPLICATION

This application claims the benefit of priority to U.S. Provisional Patent Application No. 63/406,536, filed Sep. 14, 2022.

### BACKGROUND

Phenylketonuria (PKU) is an inborn error of metabolism caused by mutations in phenylalanine hydroxylase (PAH), the enzyme responsible for metabolizing phenylalanine. PKU is an autosomal recessive metabolic disorder in which phenylalanine is not properly metabolized and results in abnormally high levels of plasma phenylalanine. People who have PKU have abnormally high blood levels of phenylalanine, which if untreated can lead to irreversible neurological damage resulting in a spectrum of complications such as intellectual disabilities, seizures, neurodevelopmental and behavioral disorders. PKU is difficult to treat because blood levels of phenylalanine are directly related to diet. Patients must adhere to a life-long and strict diet that impacts all aspects of patients' lives. Current standard of care are enzyme co-factor and enzyme substitution therapy, but these therapies are not effective in all patients, and they carry potential risk for adverse events.

The enzyme responsible for metabolizing phenylalanine, and thus maintaining phenylalanine homeostasis is phenylalanine hydroxylase (PAH). Loss-of-function (LOF) mutations at PAH gene at chromosome 12q23.2 are known to cause most forms of PKU. These LOF mutations resulting in PKU can be diagnosed as classical PKU (the most severe form), and “mild PKU” or “hyperphe” a less severe form. In addition to PAH, mutations in other enzymes that affect phenylalanine metabolism, such as dihydropteridine reductase (DHPR), the enzyme responsible for synthesis of co-factors required for PAH activity, may also result in elevated levels of phenylalanine. In addition to diet, blood amino acid levels, including levels of phenylalanine, are regulated by SLC6A19. SLC6A19 is located in the proximal tubule of the kidney and is responsible for reabsorption of amino acids back into the blood.

### SUMMARY

One aspect of the invention provides compounds, compositions, and methods useful for treating or preventing a disease or disorder associated with abnormal levels of amino acids by modulation of SLC6A19 transport.

Accordingly, provided herein is a compound having the structure of Formula (I):

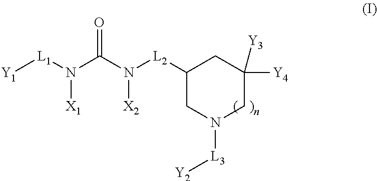

wherein:
n is 0, 1, or 2;
L₁ is absent or selected from -alkyl-, -hydroxyalkyl-, -cycloalkyl-, and -heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cyanoalkyl, —O-alkoxyalkyl, —O-haloalkyl, —O-hydroxyalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, haloalkyl, and cycloalkyl;
each Y₂″ is independently alkyl, or both instances taken together with the nitrogen atom to which they are bonded form a 4-, 5- or 6-membered heterocyclyl; and
Y₃ and Y₄ together with the carbon to which they are bonded form a 4-, 5-, or 6-membered cycloalkyl, cycloheteroalkyl, or heterocyclyl, or Y₃ and Y₄ are each independently selected from —OH, —CN, —CO₂H, —CO₂(alkyl), alkyl, hydroxyalkyl, cyanoalkyl, and halogen;
or a pharmaceutically acceptable salt thereof.

Also provided herein is a compound having the structure of Formula (II):

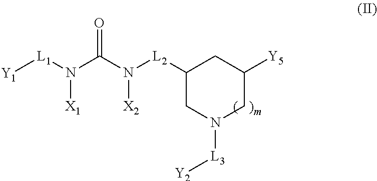

wherein:
m is 0, 1, or 2;
L₁ is absent or selected from -alkyl-, -hydroxyalkyl-, -cycloalkyl-, -heteroaryl-, and -heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cyanoalkyl, —O-alkoxyalkyl, —O-haloalkyl, —O-hydroxyalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, haloalkyl, and cycloalkyl;
each Y₂″ is independently alkyl, or both instances taken together with the nitrogen atom to which they are bonded form a 4-, 5- or 6-membered heterocyclyl;
Y₅ is selected from cycloalkyl, heteroaryl, heterocyclyl, C₀-C₆ alkyl-Y₅′, and C₂-C₆ alkenyl-Y₅′;
Y₅′ is selected from —CN, —OH, —NH₂, —OSO₂-alkyl, —NH(Y₅″), —C(O)N(Y₅′″)₂, —SO₂N(Y₅′″)₂, —O(CO)—Y₅′″, —(CO)O—Y₅′″, alkoxy, benzyloxy, —C═N—O(alkyl), and a squaramide moiety;
Y₅″ is selected from alkyl, —C(O)-alkyl, and —SO₂-alkyl; and
Y₅′″ is independently for each occurrence selected from —H, alkyl aminoalkyl, and aryl;
or a pharmaceutically acceptable salt thereof.

Further provided herein is a compound having the structure of Formula (III):

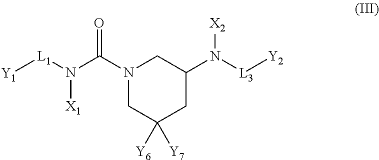

wherein:
L₁ is absent or selected from -alkyl-, -hydroxyalkyl-, -cycloalkyl-, and -heteroaryl-CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl; and
Y₆ and Y₇ together with the carbon to which they are bonded form a 4-, 5-, or 6-membered cycloalkyl or heterocyclyl;
or a pharmaceutically acceptable salt thereof.

Still further provided herein is a compound having the structure of Formula (IV):

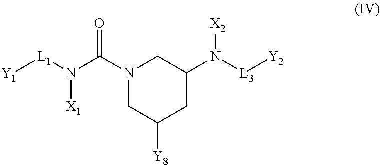

wherein:
L₁ is absent or selected from -alkyl-, -hydroxyalkyl-, -cycloalkyl-, -heteroaryl-, and
-heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl;
Y₈ is selected from cyano, cycloalkyl, heteroaryl, heterocyclyl, alkyl-Y₈′, and a squaramide moiety;
Y₈′ is selected from —CN, —OH, —NH₂, —NH(Y₈″), —C(O)N(Y₈′″)₂, —SO₂N(Y₈′″)₂, and a squaramide moiety;
Y₈″ is selected from alkyl, —C(O)-alkyl, and —SO₂-alkyl; and
Y₈′″ is independently for each occurrence selected from —H and alkyl;
or a pharmaceutically acceptable salt thereof.

Another aspect of the invention relates to methods of treating or preventing a disease or disorder associated with a genetic defect in phenylalanine hydroxylase in a subject in need thereof comprising administering to the subject an effective amount of a compound of Formula (I), (II), (III), or (IV).

Another aspect of the invention relates to methods of treating or preventing phenylketonuria, hyperphenylalaninemia, tyrosinemia, nonketotic hyperglycinemia, isovaleric acidemia, methylmalonic acidemia, propionic acidemia, maple syrup urine disease, DNAJC12 deficiency, urea cycle disorders, or hyperammonemia in a subject in need thereof comprising administering to the subject an effective amount of a compound of Formula (I), (II), (III), or (IV).

Another aspect of the invention relates to methods of modulating SLC6A19 transport in a subject in need thereof comprising administering to the subject an effective amount of a compound of Formula (I), (II), (III), or (IV).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 is a table summarizing isoleucine transport data for exemplary compounds of the invention. A=IC₅₀<500 nM; B=IC₅₀ 500 nM-1500 nM; C=IC₅₀>1500 nM-5000 nM; D=IC₅₀>5000 nM-10000 nM; and E=IC₅₀>10000 nM.

FIG. 2 is a table summarizing isoleucine transport data for additional exemplary compounds of the invention. A=IC₅₀<500 nM; B=IC₅₀ 500 nM-1500 nM; C=IC₅₀>1500 nM-5000 nM; D=IC₅₀>5000 nM-10000 nM; and E=IC₅₀>10000 nM.

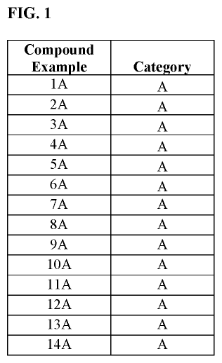

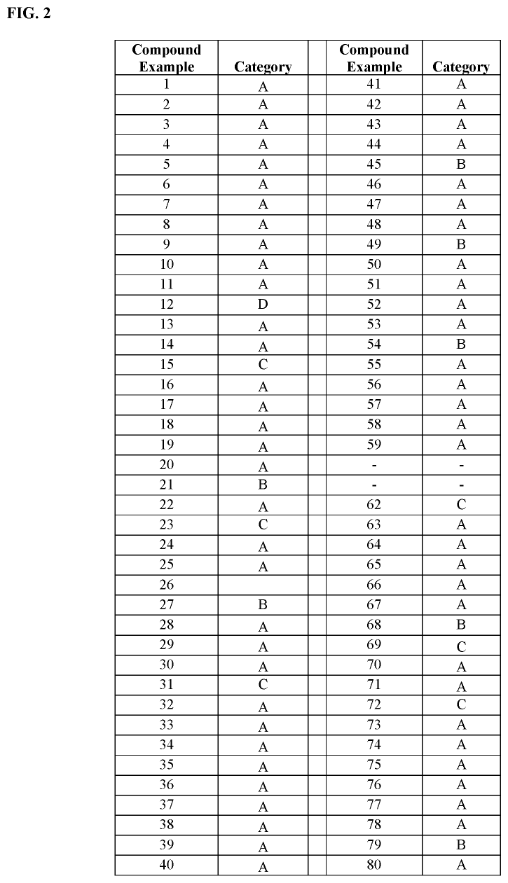

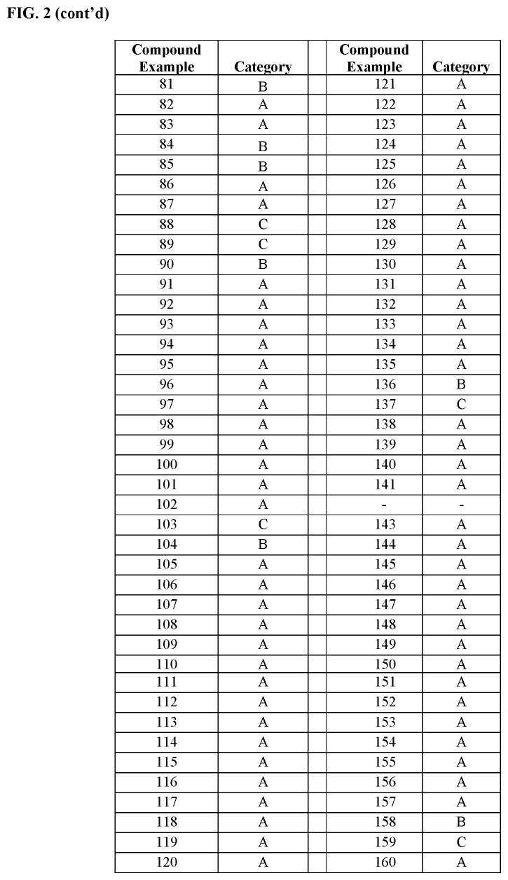

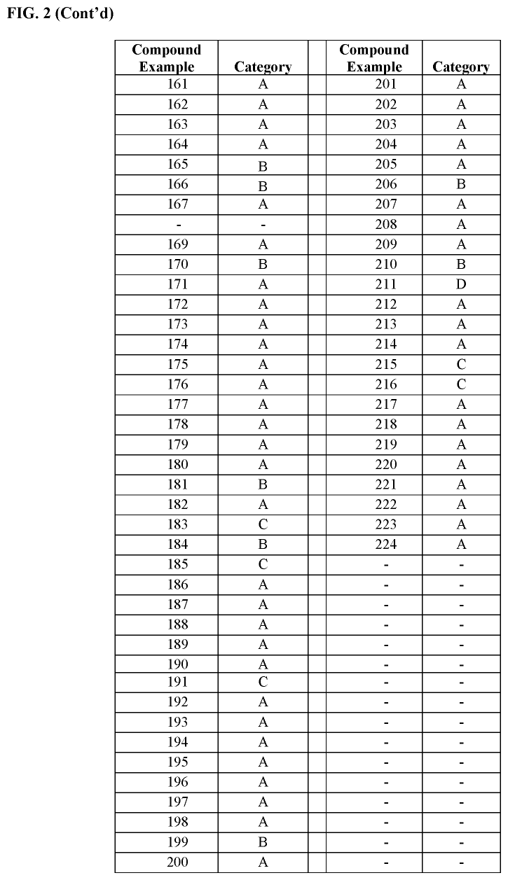

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “tautomer” as used herein means structural isomers that exist in equilibrium resulting from the migration of a hydrogen atom. For example, the two tautomers of 2-pyrimidinone are recited below. A single tautomer may be provided in a structural representation of a given compound. However, the present invention contemplates all such tautomers of a given compound.

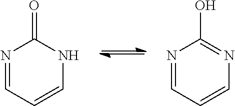 [SMILES: O=c1nccc[nH]1 ; Oc1ncccn1]

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to a cycloalkyl group as hereinbefore defined substituted with at least one halogen.

“Cycloheteroalkyl” refers to an cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carbocyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

### Formulas (I) and (II)

Provided herein is a compound having the structure of Formula (I):

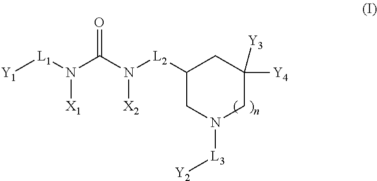

wherein:
n is 0, 1, or 2;
L₁ is absent or selected from -alkyl-, -hydroxyalkyl-, -cycloalkyl-, and -heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cyanoalkyl, —O-alkoxyalkyl, —O-haloalkyl, —O-hydroxyalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, haloalkyl, and cycloalkyl;
each Y₂″ is independently alkyl, or both instances taken together with the nitrogen atom to which they are bonded form a 4-, 5- or 6-membered heterocyclyl; and
Y₃ and Y₄ together with the carbon to which they are bonded form a 4-, 5-, or 6-membered cycloalkyl, cycloheteroalkyl, or heterocyclyl, or Y₃ and Y₄ are each independently selected from —OH, —CN, —CO₂H, —CO₂(alkyl), alkyl, hydroxyalkyl, cyanoalkyl, and halogen;
or a pharmaceutically acceptable salt thereof.

In certain embodiments,
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl; and
Y₃ and Y₄ together with the carbon to which they are bonded form a 4-, 5-, or 6-membered cycloalkyl, cycloheteroalkyl, or heterocyclyl. In certain embodiments, Y₃ and Y₄ together with the carbon to which they are bonded form an unsubstituted 4-, 5-, or 6-membered heterocyclyl.

In certain embodiments, Y₃ and Y₄ together with the carbon to which they are bonded form an unsubstituted 4-, 5-, or 6-membered cyclic urea, cyclic carbamate, cyclic sulfone, cyclic sulfonamide, lactam, azalactam, or lactone.

In certain embodiments, Y₃ and Y₄ together with the carbon to which they are bonded form any one of the following structures:

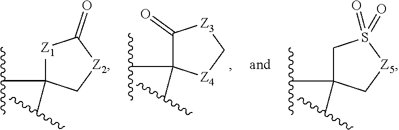

wherein Z₁ is selected from O, NH, and CH₂; Z₂ is selected from O, NH, and CH₂; Z₃ is selected from O and NH; Z₄ is selected from NH and CH₂; and Z₅ is selected from NH and CH₂; provided that one of Z₁ and Z₂ is not CH₂.

In certain embodiments, Y₃ and Y₄ together with the carbon to which they are bonded form the following structure:

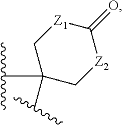

wherein Z₁ is selected from O, NH, and CH₂; and Z₂ is selected from O, NH, and CH₂; provided that one of Z₁ and Z₂ is not CH₂.

In certain embodiments, Y₃ and Y₄ together with the carbon to which they are bonded form any one of the following structures:

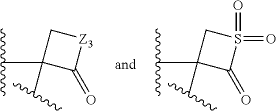

wherein Z₃ is selected from O and NH; and Z₅ is selected from NH and CH₂.

In certain embodiments, Y₃ and Y₄ together with the carbon to which they are bonded form an unsubstituted 4-, 5-, or 6-membered cycloheteroalkyl.

In certain embodiments, Y₃ and Y₄ together with the carbon to which they are bonded form an unsubstituted piperidinyl, tetrahydrofuranyl, azetidinyl, or morpholinyl.

In certain embodiments, Y₃ and Y₄ together with the carbon to which the are bonded form any one of the following structures:

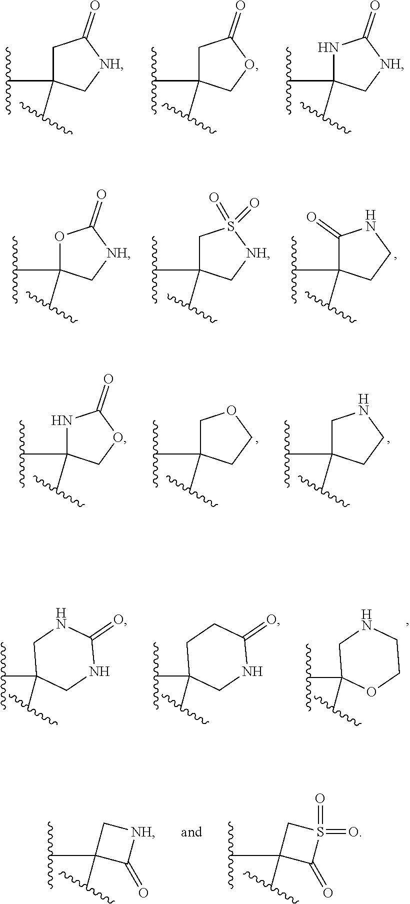

In certain embodiments Y₃ and Y₄ together with the carbon to which they are bonded form the following structure:

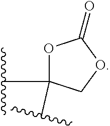 [SMILES: *C1(*)COC(=O)O1]

In certain embodiments, Y₃ and Y₄ together with the carbon to which they are bonded form a substituted 4-, 5-, or 6-membered heterocyclyl.

In certain embodiments, Y₃ and Y₄ together with the carbon to which they are bonded form a substituted 4-, 5-, or 6-membered cyclic urea, cyclic carbamate, cyclic sulfone, cyclic sulfonamide, lactam, azalactam, or lactone.

In certain embodiments, the cyclic urea, cyclic carbamate, cyclic sulfonamide, lactam, or azalactam is N-substituted.

In certain embodiments, the cyclic urea, cyclic carbamate, cyclic sulfonamide, lactam, or azalactam is N-alkyl substituted.

In certain embodiments, Y₃ and Y₄ together with the carbon to which they are bonded form the following structure:

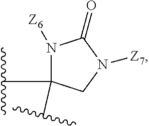

wherein Z₆ is selected from —H and alkyl; Z₇ is selected from —H and alkyl;
provided that Z₆ and Z₇ are not both —H.

In certain embodiments, Y₃ and Y₄ together with the carbon to which they are bonded form any one of the following structures:

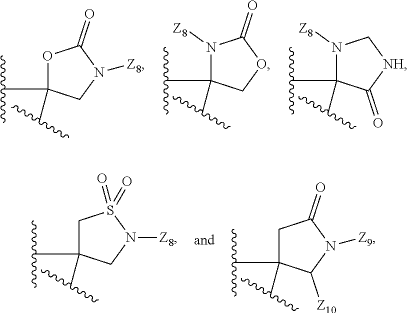

wherein each Z₈ is independently an alkyl; and Z₉ is selected from —H and alkyl; and Z₁₀ is selected from —H and alkyl; provided that Z₉ and Z₁₀ are not both —H.

In certain embodiments, Y₃ and Y₄ together with the carbon to which they are bonded form the following structure:

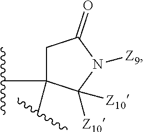

Z₉ is selected from —H and alkyl; and each Z₁₀′ is an alkyl or together with the carbon to which they are bonded from an unsubstituted or substituted cycloalkyl, e.g. cyclopropyl.

In certain embodiments, Y₃ and Y₄ together with the carbon to which they are bonded form the following structure:

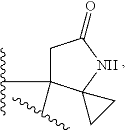 [SMILES: *C1(*)CC(=O)NC12CC2]

In certain embodiments, Y₃ and Y₄ together with the carbon to which they are bonded form any one of the following structures:

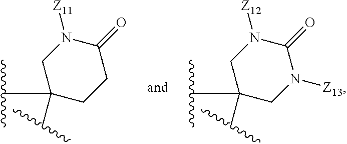

wherein Z₁₁ is alkyl; Z₁₂ is selected from —H and alkyl; and Z₁₃ is selected from —H and alkyl; provided that Z₁₂ and Z₁₃ are not both —H.

In certain embodiments Y₃ and Y₄ together with the carbon to which they are bonded form the following structure:

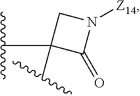

wherein Z₁₄ is alkyl.

In certain embodiments, Y₃ and Y₄ together with the carbon to which they are bonded form a substituted 4-, 5-, or 6-membered cycloheteroalkyl.

In certain embodiments, Y₃ and Y₄ together with the carbon to which they are bonded form a substituted piperidinyl, tetrahydrofuranyl, azetidinyl, or morpholinyl.

In certain embodiments, Y₃ and Y₄ together with the carbon to which they are bonded form an N-alkyl or N-acetyl substituted piperidinyl, azetidinyl, or morpholinyl.

In certain embodiments, Y₃ and Y₄ together with the carbon to which they are bonded form a substituted 4-, 5-, or 6-membered cycloalkyl. In certain embodiments, the substituted 4-, 5-, or 6-membered cycloalkyl is substituted with —CN, alkyl or hydroxyalkyl.

In certain embodiments, Y₃ and Y₄ together with the carbon to which they are bonded form a substituted cyclopropyl. In certain embodiments, Y₃ and Y₄ together with the carbon to which they are bonded form a substituted cyclobutyl.

In certain embodiments, Y₃ and Y₄ together with the carbon to which they are bonded form any one of the following structures:

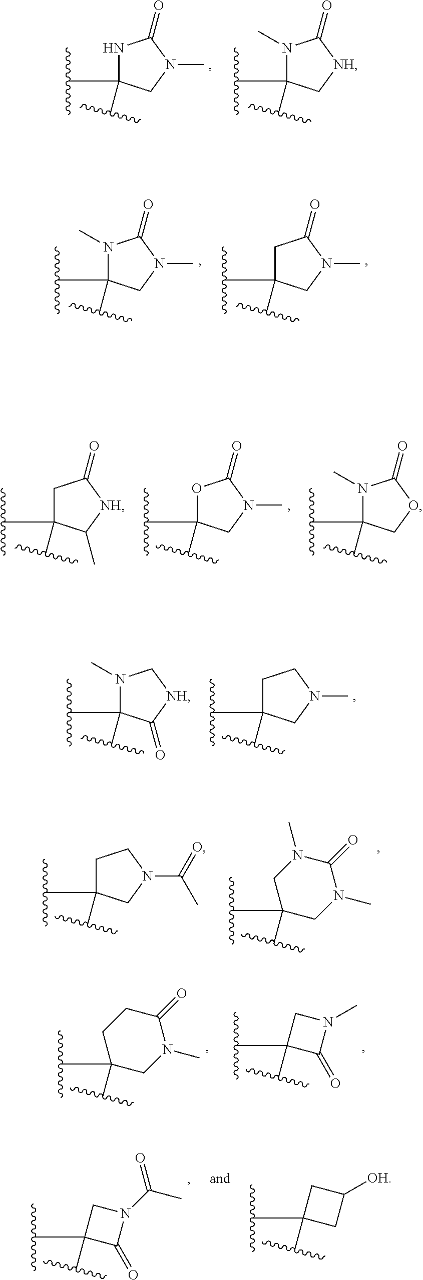

In certain embodiments, Y₃ and Y₄ together with the carbon to which they are bonded form the following structure:

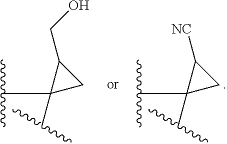 [SMILES: *C1(*)CC1CO ; *C1(*)CC1C#N]

In certain embodiments, Y₃ and Y₄ together with the carbon to which they are bonded form an unsubstituted 4-, 5-, or 6-membered cycloheteroalkyl.

In certain embodiments, Y₃ and Y₄ together with the carbon to which they are bonded form a substituted tetrahydrofuranyl or tetrahydropyranyl.

In certain embodiments, Y₃ and Y₄ together with the carbon to which they are bonded form any one of the following structures:

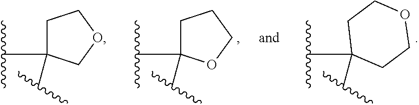

In certain embodiments, Y₃ and Y₄ are each independently selected from —OH, —CN, —CO₂H, —CO₂(alkyl), alkyl, hydroxyalkyl, cyanoalkyl, and halogen;

In certain embodiments, Y₃ and Y₄ are each independently selected from —F, —OH, —CN, —CO₂H, —CO₂Et, —CH₃, —CH₂CH₃, —CH₂CN, —CH₂OH, and —CH₂OSO₂Me.

In certain embodiments, Y₃ is selected from —F, CH₃, and —CH₂CH₃; and Y₄ is selected from —OH, —CN, —CO₂H, —CO₂Et, —CH₂CN, —CH₂OH, and —CH₂OSO₂Me.

In certain embodiments, n is 0. In other embodiments, n is 1. In other embodiments, n is 2. In certain embodiments, the compound having the structure selected from:

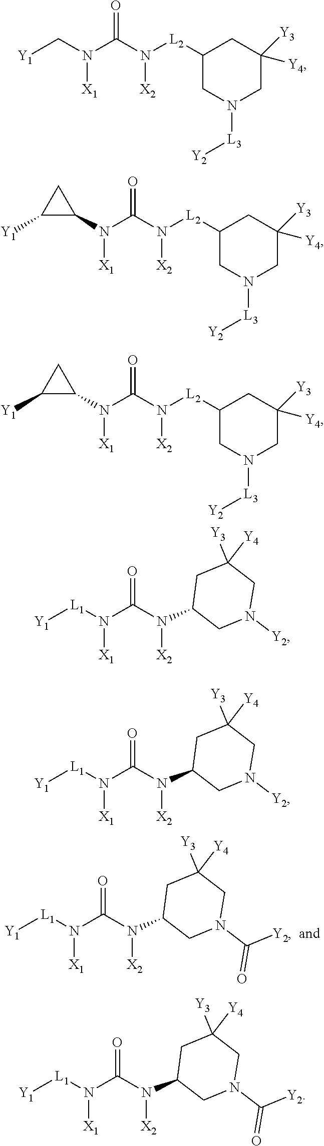

Also provided herein is a compound having the structure of Formula (II):

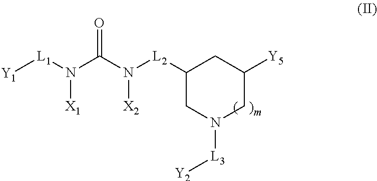

wherein:
m is 0, 1, or 2;
L₁ is absent or selected from -alkyl-, -hydroxyalkyl-, -cycloalkyl-, -heteroaryl-, and -heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cyanoalkyl, —O-alkoxyalkyl, —O-haloalkyl, —O-hydroxyalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, haloalkyl, and cycloalkyl;
each Y₂″ is independently alkyl, or both instances taken together with the nitrogen atom to which they are bonded form a 4-, 5- or 6-membered heterocyclyl;
Y₅ is selected from cycloalkyl, heteroaryl, heterocyclyl, C₀-C₆ alkyl-Y₅′, and C₂-C₆ alkenyl-Y₅′;
Y₅′ is selected from —CN, —OH, —NH₂, —OSO₂-alkyl, —NH(Y₅″), —C(O)N(Y₅′″)₂, —SO₂N(Y₅′″)₂, —O(CO)—Y₅′″, —(CO)O—Y₅′″, alkoxy, benzyloxy, —C═N—O(alkyl), and a squaramide moiety;
Y₅″ is selected from alkyl, —C(O)-alkyl, and —SO₂-alkyl; and
Y₅′″ is independently for each occurrence selected from —H, alkyl, aminoalkyl, and aryl;
or a pharmaceutically acceptable salt thereof.

In certain embodiments,
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl;
Y₅ is selected from cyano, cycloalkyl, heteroaryl, heterocyclyl, alkyl-Y₅′, and a squaramide moiety;
Y₅′ is selected from —CN, —OH, —NH₂, —NH(Y₅″), —C(O)N(Y₅′″)₂, —SO₂N(Y₅′″)₂, —and a squaramide moiety;
Y₅″ is selected from alkyl, —C(O)-alkyl, and —SO₂-alkyl; and
Y₅′″ is independently for each occurrence selected from —H and alkyl.

In certain embodiments, the compound having structure:

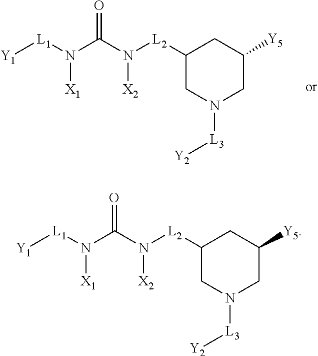

In certain embodiments, Y₅ is an unsubstituted 5-membered heteroaryl.

In certain embodiments, Y₅ is selected from an unsubstituted pyrazolyl, unsubstituted diazolyl, unsubstituted oxazolyl, and unsubstituted isooxazolyl.

In certain embodiments, Y₅ is selected from

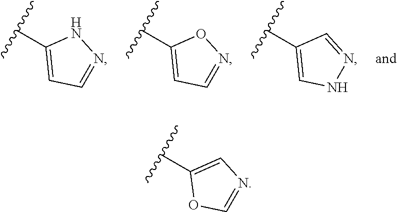 [SMILES: *c1ccn[nH]1 ; *c1ccno1 ; *c1cn[nH]c1 ; "and" ; *c1cnco1]

In certain embodiments, Y₅ is a substituted 6-membered heteroaryl.

In certain embodiments, Y₅ is selected from substituted pyridinyl and substituted pyrimidinyl.

In certain embodiments, Y₅ is selected from

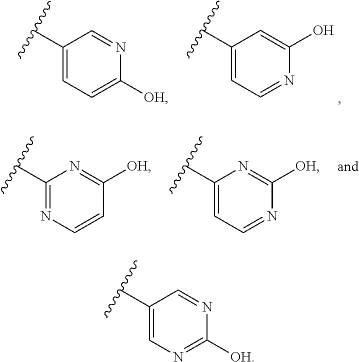 [SMILES: *c1ccc(O)nc1 ; *c1ccnc(O)c1 ; *c1nccc(O)n1 ; *c1ccnc(O)n1 ; "and" ; *c1cnc(O)nc1]

In certain embodiments, Y₅ is alkyl-Y₅′.

In certain embodiments, Y₅ is C₁-C₄ alkyl-Y₅′; and the alkyl is unbranched.

In certain embodiments, Y₅ is C₁-C₄ alkyl-Y₅′; and the alkyl is branched.

In certain embodiments, Y₅ is C₁-C₄ alkyl-Y₅′; and the alkyl is substituted with a cycloalkyl.

In certain embodiments, Y₅′ is selected from —NH(Y₅″), —C(O)N(Y₅′″)₂, and —SO₂N(Y₅′″)₂; Y₅″ is selected from —C(O)—CH₃, and —SO₂—CH₃; and Y₅″ is independently for each occurrence selected from —H and —CH₃.

In certain embodiments, Y₅′ is —OH, —CN, or alkoxy.

In certain embodiments, Y₅′ is O(CO)—Y₅″ or —(CO)O—Y₅′″.

In certain embodiments, Y₅′″ is alkyl, aminoalkyl, or aryl. In certain embodiments, Y₅′ is a squaramide moiety.

In certain embodiments, Y₅′ is

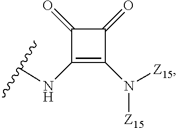

wherein Z₁₅ is independently for each occurrence selected from —H and alkyl.

In certain embodiments, each Z₁₅ is —H, each Z₁₅ is —CH₃, or one Z₁₅ is —H and the other is —CH₃.

In certain embodiments, Y₅ is a squaramide moiety.

In certain embodiments, Y₅ is

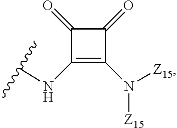

wherein Z₁₅ is independently for each occurrence selected from —H and alkyl.

In certain embodiments, each Z₁₅ is —H, each Z₁₅ is —CH₃, or one Z₁₅ is —H and the other is —CH₃.

In certain embodiments, Y₅ is selected from

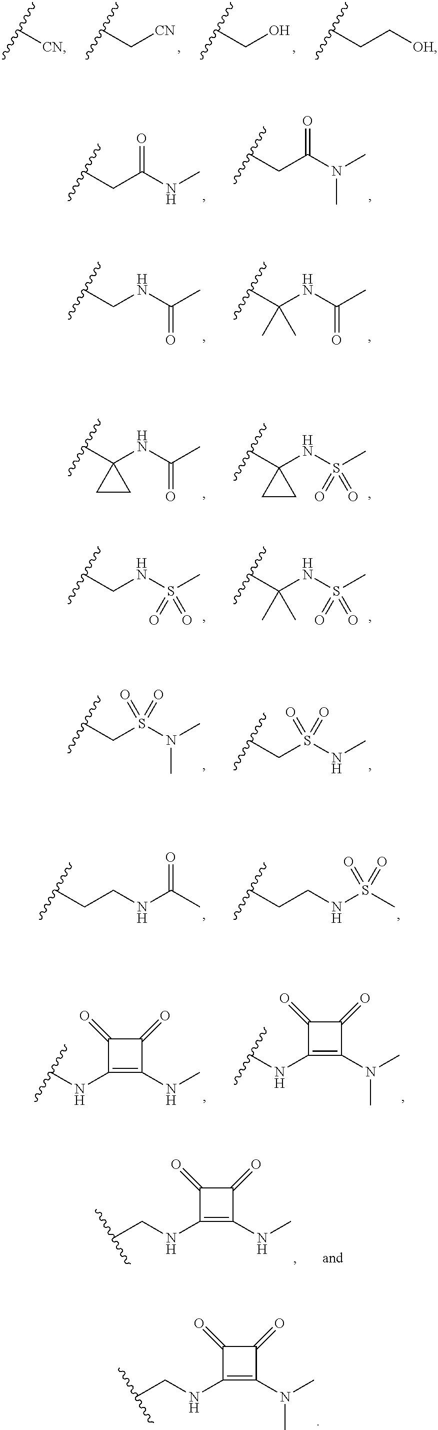

In certain embodiments, Y₅ is selected from —OH, —OAc,

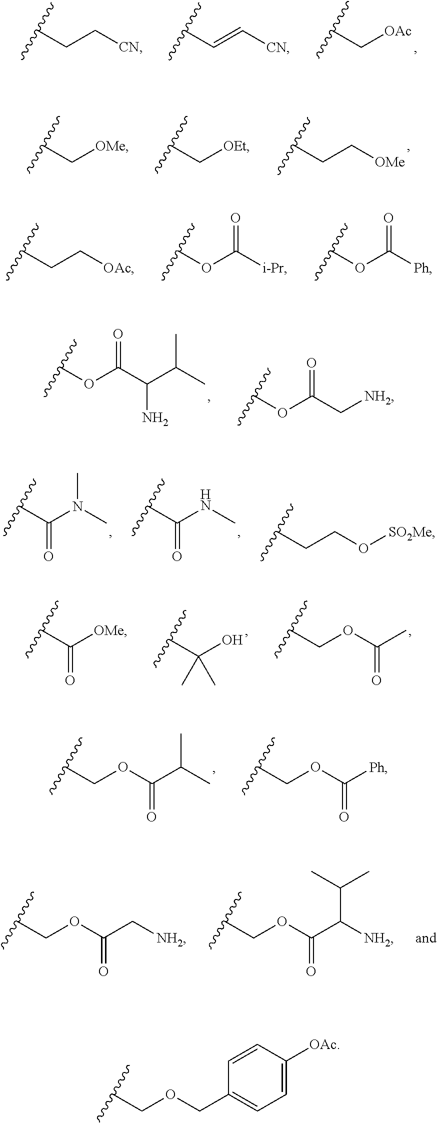

In certain embodiments, m is 0. In other embodiments, m is 1. In other embodiments, m is 2.

In certain embodiments, the compound having the structure:

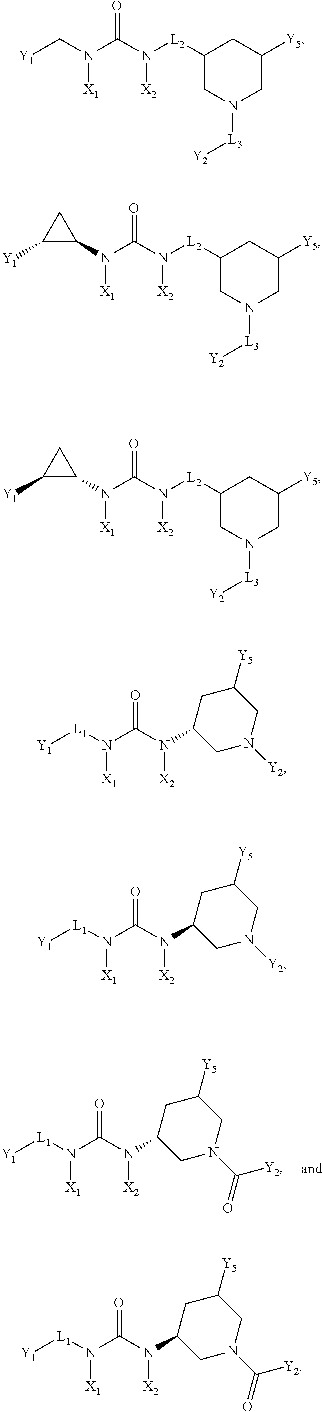

### Further Embodiments of Formulas (I) and (II)

In certain embodiments, one of X₁ and X₂ is —H; and the other of X₁ and X₂ is selected from —CH₃, —CH₂CH₃, —CH₂CF₃, —CH₂CH₂CH₃,

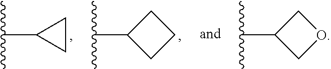

In certain embodiments, X₁ is —H; and X₂ is

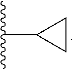 [SMILES: *C1CC1]

In certain embodiments, X₁ is —H; and X₂ is —CH₃. In other embodiments, X₁ is —H; and X₂ is —CH₂CH₃. In other embodiments, X₁ is —H; and X₂ is —CH₂CH₂CH₃.

In certain embodiments, X₂ is —H; and X₁ is

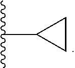 [SMILES: *C1CC1]

In certain embodiments, X₂ is —H; and X₁ is —CH₃. In other embodiments, X₂ is —H; and X₁ is —CH₂CH₃. In other embodiments, X₂ is —H; and X₁ is —CH₂CH₂CH₃.

In certain embodiments, L₁ is absent.

In certain embodiments, L₁ is selected from -alkyl-, -hydroxyalkyl-, -cycloalkyl-, -heteroaryl-, and -heteroaryl-CH₂—.

In certain embodiments, L₁ is selected from —CH₂—, —C(H)(CH₃)—, —CH₂CH₂—, and —C(H)(OH)CH₂—.

In certain embodiments, L₁ is

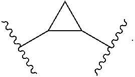 [SMILES: *C1CC1*]

In certain embodiments, L₁ is selected from

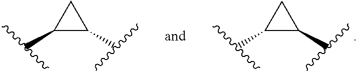 [SMILES: *[C@H]1C[C@@H]1* ; *[C@@H]1C[C@H]1*]

In certain embodiments, L₁ is selected from

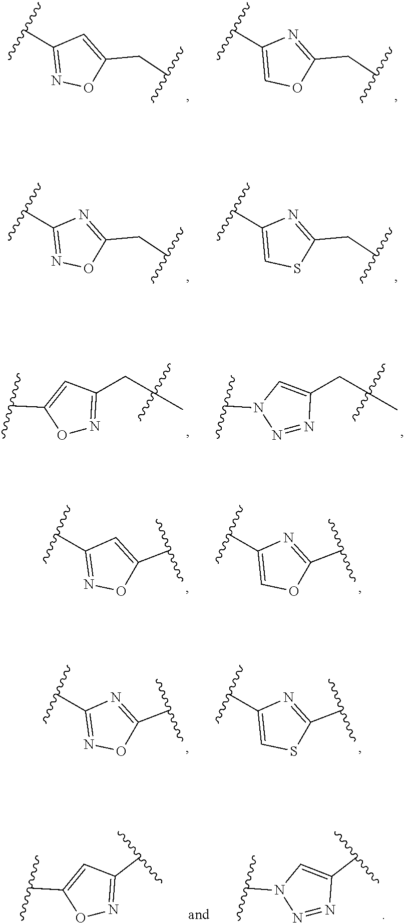

In certain embodiments, Y₁ is unsubstituted aryl. In other embodiments, Y₁ is selected from unsubstituted phenyl and unsubstituted naphthyl.

In certain embodiments, Y₁ is substituted aryl.

In certain embodiments, Y₁ is

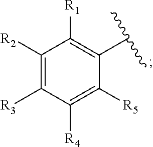

and
R₁, R₂, R₃, R₄, and R₅ are independently selected from —H, halogen, —CN, —CF₃, —CHF₂, —CF₂CH₃, —OCF₃, —OCHF₂, alkyl, haloalkyl, alkenyl, alkynyl, alkoxy, cycloalkyl, heterocyclyl, aryl, and heteroaryl; provided that one of R₁, R₂, R₃, R₄, and R₅ is not —H.

In certain embodiments, R₁, R₂, R₃, R₄, and R₅ are independently selected from —H, —F, —Cl, —Br, —CN, —CH₃, —CH₂CH₃, —CF₃, —CHF₂, —CF₂CH₃, —OCH₃, —OCF₃, —OCHF₂,

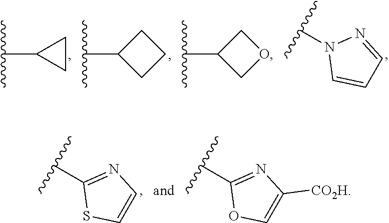

In certain embodiments, R₁, R₂, R₃, R₄, and R₅ are independently selected from —H, —F, —Cl, —Br, —CN, —CH₃, —CH₂CH₃, —CH₂CH₂CH₃, —CH(CH₃)₂, —OCH₃, —OCF₃, and

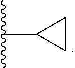 [SMILES: *C1CC1]

In certain embodiments, two of R₁, R₂, R₃, R₄, and R₅ are not —H, or three of R₁, R₂, R₃, R₄, and R₅ are not —H.

In certain embodiments, Y₁ is selected from

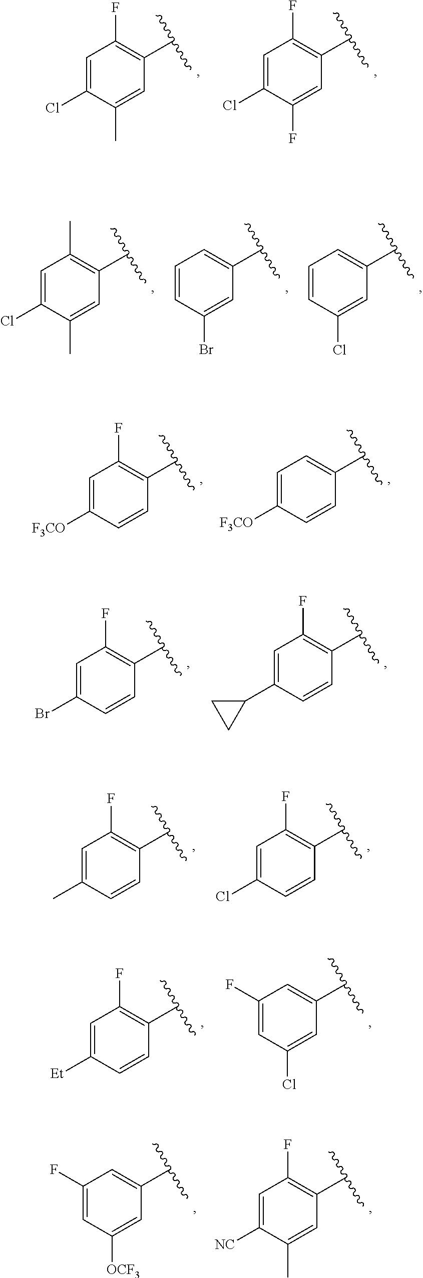

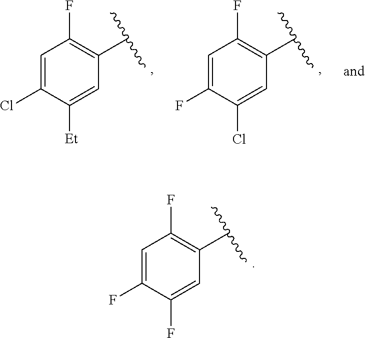 [SMILES: *c1cc(CC)c(Cl)cc1F ; *c1cc(Cl)c(F)cc1F ; *c1cc(F)c(F)cc1F]

In certain embodiments, Y₁ is

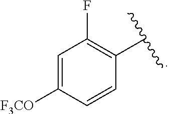 [SMILES: *c1ccc(OC(F)(F)F)cc1F]

In certain embodiments, Y₁ is unsubstituted heteroaryl.

In certain embodiments Y₁ is selected from

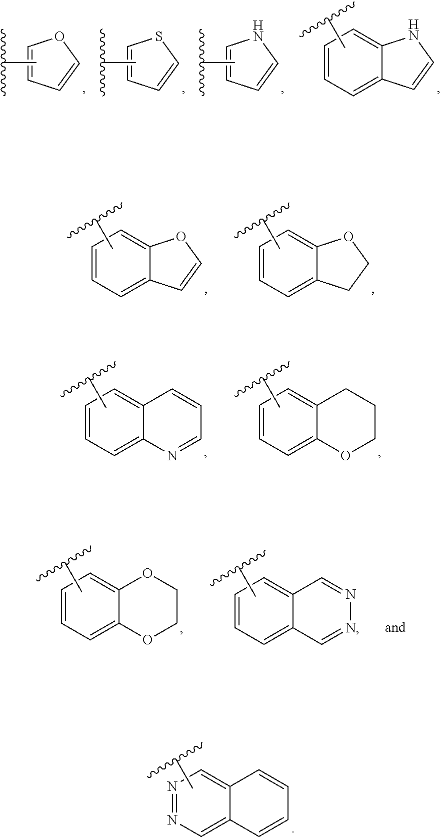

In certain embodiments, Y₁ is substituted heteroaryl.

In certain embodiments, Y₁ is selected from

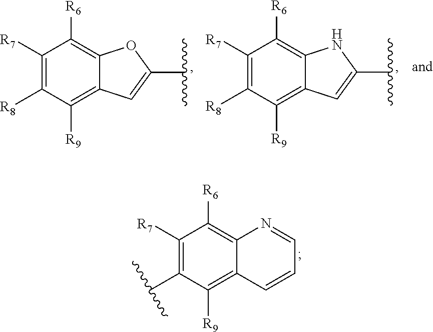

and
each occurrence of R₆, R₇, R₈, and R₉ are independently selected from —H, halogen, —CN, —OCF₃, —OCHF₂, alkyl, haloalkyl, alkenyl, alkynyl, alkoxy, cycloalkyl, aryl, and heteroaryl; provided that at least one of R₆, R₇, R₈, and R₉ is not —H.

In certain embodiments, L₂ is absent.

In certain embodiments, L₂ is —CH₂—.

In certain embodiments, L₃ is absent.

In certain embodiments, Y₂ is unsubstituted heteroaryl.

In certain embodiments, Y₂ is selected from

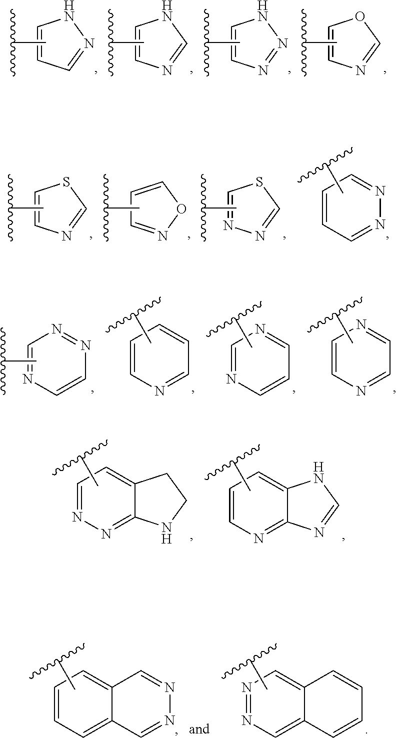

In certain embodiments, Y₂ is

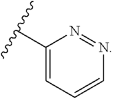 [SMILES: *c1cccnn1]

In certain embodiments, Y₂ is substituted heteroaryl.

In certain embodiments, Y₂ is

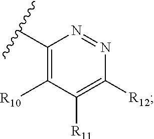

R₁₀, R₁₁, and R₁₂ are independently selected from —H, halogen, —CN, —OH, —NH₂, —OCF₃, —OCHF₂, —OAc, —NHAc, alkyl, haloalkyl, hydroxyalkyl, alkenyl, alkynyl, alkoxy, alkylamino cycloalkyl, aryl, heteroaryl, —C(O)NR₁₃R₁₄, —CO₂R₁₅, and —C(O)NHSO₂R₁₅; provided that at least one of R₁₀, R₁₁, and R₁₂ is not —H; and
each occurrence of R₁₃, R₁₄, and R₁₅ is independently selected from —H, alkyl, aryl, and heteroaryl.

In certain embodiments, R₁₀, R₁₁, and R₁₂ are independently selected from —H, —F, —Cl, —Br, —CN, —CH₃, —CH₂CH₃, —CF₃, —CHF₂, —CF₂CH₃, —OCH₃, —OCF₃, —OCHF₂, —OAc, —NH₂, —NHCH₃, —NHAc, —C(O)NH₂, —C(O)NHCH₃, —C(O)NHCH₂CH₃, —C(O)NHSO₂CH₃, —C(O)NHSO₂CH₂CH₃, —CH₂OH, —CO₂H, phenyl, cyclopropyl, cyclobutyl, imidazolyl, and tetrazolyl.

In certain embodiments, R₁₀ and R₁₂ are each —H; and R₁₁ is selected from —CN, —CF₃, —CH₃, —OCH₃, —NH₂, —NHCH₃, —NHAc, —CO₂H, —C(O)NH₂, —C(O)NHCH₃, —C(O)NHCH₂CH₃,

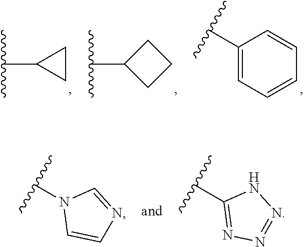

In certain embodiments, R₁₁ and R₁₂ are each —H; and R₁₀ is selected from —CN, —CF₃, —CH₃, —OCH₃, —NH₂, —NHCH₃, —NHAc, —CO₂H, —C(O)NH₂, —C(O)NHCH₃, —C(O)NHCH₂CH₃,

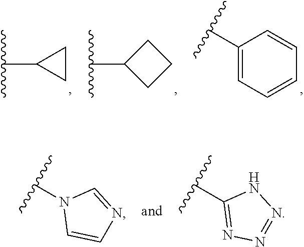

In certain embodiments, R₁₀ and R₁₁ are each —H; and R₁₂ is selected from —CN, —CF₃, —CH₃, —OCH₃, —NH₂, —NHCH₃, —NHAc, —CO₂H, —C(O)NH₂, —C(O)NHCH₃, —C(O)NHCH₂CH₃,

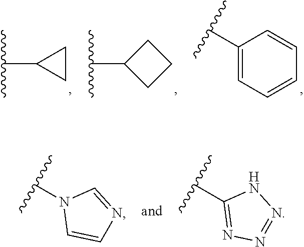

In certain embodiments, Y₂ is selected from

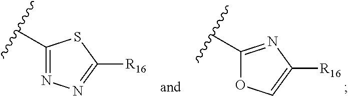

or Y₂ is selected from

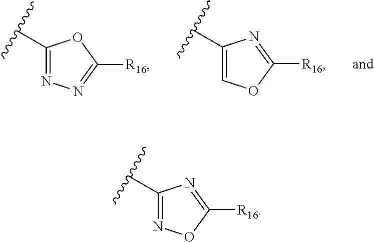

In certain embodiments, R₁₆ for each occurrence is independently selected from halogen, —CN, —NH₂, —OCF₃, —OCHF₂, —OAc, —NHAc, alkyl, haloalkyl, alkenyl, alkynyl, alkoxy, alkylamino cycloalkyl, aryl, heteroaryl, —C(O)NR₁₃R₁₄, —CO₂R₁₅; and each occurrence of R₁₃, R₁₄, and R₁₅ is independently selected from —H, alkyl, aryl, and heteroaryl. In certain embodiments, R₁₆ for each occurrence is independently selected from hydroxyalkyl and alkoxyalkyl.

In certain embodiments, R₁₆ is selected from —CN, —CH₃, —CF₃, —C(O)NH₂, —CO₂CH₂CH₃, and

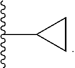 [SMILES: *C1CC1]

In certain embodiments, R₁₆ is selected from i-Pr, —CH₂OH, and —CH₂OCH₃.

In certain embodiments, Y₂ is selected from

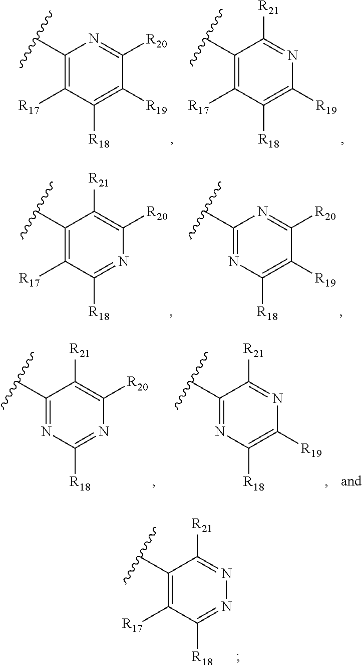

each occurrence of R₁₇, R₁₈, R₁₉, R₂₀, and R₂₁ is independently selected from —H, halogen, —CN, —NH₂, —OCF₃, —OCHF₂, —OAc, —NHAc, alkyl, haloalkyl, alkenyl, alkynyl, alkoxy, alkylamino cycloalkyl, aryl, heteroaryl, —C(O)NR₁₃R₁₄, and —CO₂R₁₅; provided that at least one of R₁₇, R₁₈, R₁₉, R₂₀, and R₂₁ is not —H; and
each occurrence of R₁₃, R₁₄, and R₁₅ is independently selected from —H, alkyl, aryl, and heteroaryl.

In certain embodiments, R₁₇, R₁₈, R₁₉, R₂₀, and R₂₁ are independently selected from —H, —CN, —CH₃, and —OCH₃.

In certain embodiments, Y₂ is selected from

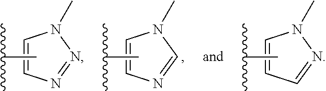

In other embodiments, Y₂ is selected from

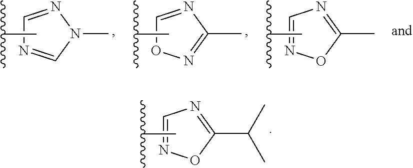

In other embodiments, Y₂ is selected from

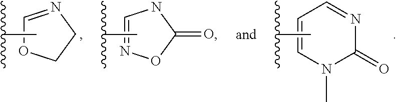

In certain embodiments, Y₂ is selected from

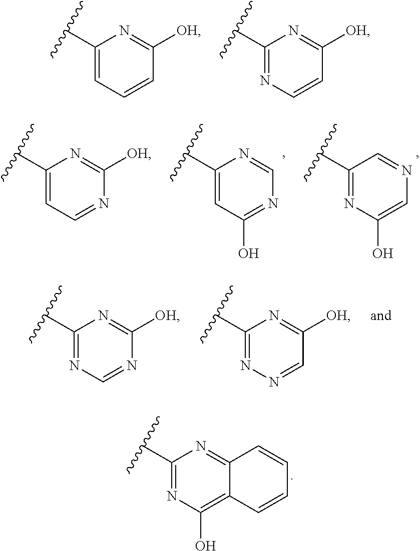

In certain embodiments, Y₂ is

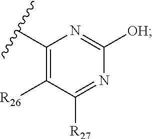

and
R₂₆ and R₂₇ are independently selected from —H, halogen, —CN, —OH, —OCF₃, —OCHF₂, —NH₂, alkyl, alkoxy, alkylamino, and cycloalkyl; provided that at least one of R₆ and R₇ is not —H; or R₆ and R₇ taken together with the carbon atoms to which they are bonded form an unsubstituted or substituted fused C₅-C₇ cycloalkyl; or
Y₂ is

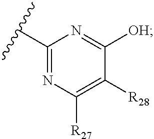

and
R₂₇ and R₂₈ are independently selected from —H, halogen, —CN, —OH, —OCF₃, —OCHF₂, —NH₂, alkyl, alkoxy, alkylamino, and cycloalkyl; provided that at least one of R₇ and R₈ is not —H; or R₇ and R₈ taken together with the carbon atoms to which they are bonded form an unsubstituted or substituted fused C₅-C₇ cycloalkyl; or
Y₂ is

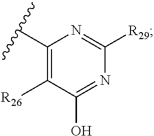

and
R₂₆ and R₂₉ are independently selected from —H, halogen, —CN, —OH, —OCF₃, —OCHF₂, —NH₂, alkyl, alkoxy, alkylamino, and cycloalkyl; provided that at least one of R₆ and R₉ is not —H; or
Y₂ is

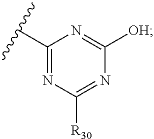

and
R₃₀ is selected from halogen, —CN, —OH, —OCF₃, —OCHF₂, —NH₂, alkyl, alkoxy, alkylamino, and cycloalkyl; or
Y₂ is

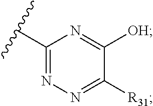

and
R₃₁ is selected from halogen, —CN, —OH, —OCF₃, —OCHF₂, —NH₂, alkyl, alkoxy, alkylamino, and cycloalkyl.

In certain embodiments, Y₂ is selected from

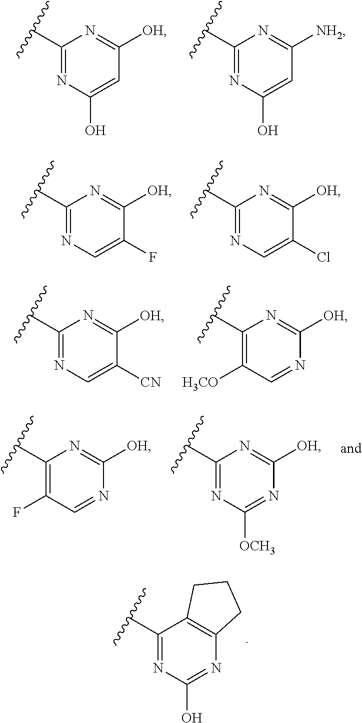

In certain embodiments, L₃ is —C(O)—.

In certain embodiments, Y₂ is selected from alkyl, alkenyl, alkynyl, alkoxy, alkoxyalkyl, hydroxyalkyl, and haloalkyl.

In certain embodiments, Y₂ is selected from alkyl, alkenyl, alkynyl, alkoxy, alkoxyalkyl, hydroxyalkyl, haloalkyl, and cyanoalkyl.
wherein Y₂ is selected from —CH₃, —CH₂CH₃, —CF₃, —CH₂CH(CH₃)₂, —CH₂CH₂C≡CH, —CH₂CH₂OCH₃, —C(H)(CH₃)CH₂OCH₃, —OCH₃, —OCH₂CH₃, —CH₂OH, —CH₂CH₂OH, —C(CH₃)₂OH, —CH₂CH₂F, —CH₂CH₂CN, and —CH₂OCH₃.

In certain embodiments, Y₂ is selected from —CH₃, —CF₃, —CH₂CH(CH₃)₂, —CH₂CH₂C≡CH, —CH₂CH₂OCH₃, —C(H)(CH₃)CH₂OCH₃, —OCH₃, —CH₂OH, —CH₂CH₂OH, —C(CH₃)₂OH, and —CH₂OCH₃.

In certain embodiments, Y₂ is selected from —CH₂OH and —CH₂CH₂OH.

In certain embodiments, Y₂ is unsubstituted heteroaryl.

In certain embodiments, Y₂ selected from,

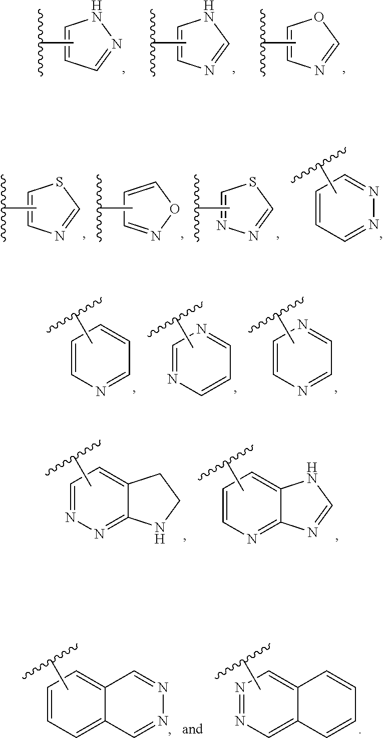

In certain embodiments, Y₂ is

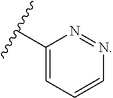 [SMILES: *c1cccnn1]

In certain embodiments, Y₂ is substituted heteroaryl.

In certain embodiments, Y₂ is selected from

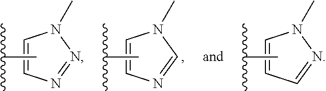

In certain embodiments, Y₂ is

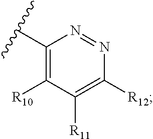

R₁₀, R₁₁, and R₁₂ are independently selected from —H, halogen, —CN, —OH, —NH₂, —OCF₃, —OCHF₂, —OAc, —NHAc, alkyl, haloalkyl, hydroxyalkyl, alkenyl, alkynyl, alkoxy, alkylamino, cycloalkyl, aryl, heteroaryl, —C(O)NR₁₃R₁₄, and —CO₂R₁₅; provided that at least one of R₁₀, R₁₁, and R₁₂ is not —H; and each occurrence of R₁₃, R₁₄, and R₁₅ is independently selected from —H, alkyl, aryl, and heteroaryl.

In certain embodiments, R₁₀, R₁₁, and R₁₂ are independently selected from —H, —F, —Cl, —Br, —CN, —CH₃, —CH₂CH₃, —CF₃, —CHF₂, —CF₂CH₃, —OCH₃, —OCF₃, —OCHF₂, —OAc, —NH₂, —NHCH₃, —NHAc, —C(O)NH₂, —C(O)NHCH₃, —C(O)NHCH₂CH₃, —C(O)NHSO₂CH₃, —C(O)NHSO₂CH₂CH₃, —CH₂OH, —CO₂H, phenyl, cyclopropyl, cyclobutyl, imidazolyl, and tetrazol 1.

In certain embodiments, Y₂

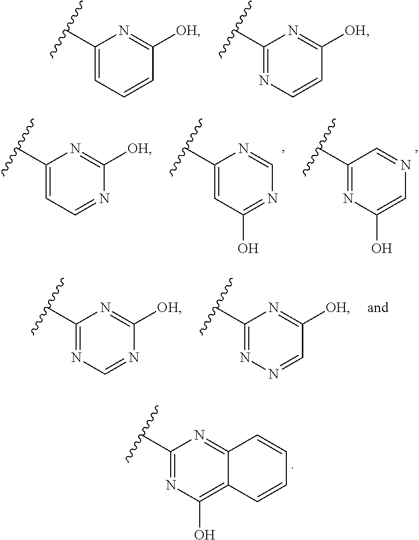

In certain embodiments, Y₂ is

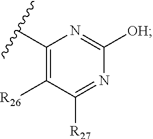

and
R₂₆ and R₂₇ are independently selected from —H, halogen, —CN, —OH, —OCF₃, —OCHF₂, —NH₂, alkyl, alkoxy, alkylamino, and cycloalkyl; provided that at least one of R₆ and R₇ is not —H; or R₆ and R₇ taken together with the carbon atoms to which they are bonded form an unsubstituted or substituted fused C₅-C₇ cycloalkyl; or

### Y₂ is

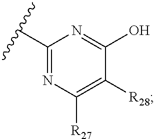

and
R₂₇ and R₂₈ are independently selected from —H, halogen, —CN, —OH, —OCF₃, —OCHF₂, —NH₂, alkyl, alkoxy, alkylamino, and cycloalkyl; provided that at least one of R₇ and R₈ is not —H; or R₇ and R₈ taken together with the carbon atoms to which they are bonded form an unsubstituted or substituted fused C₅-C₇ cycloalkyl; or

### Y₂ is

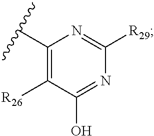

and
R₂₇ and R₂₈ are independently selected from —H, halogen, —CN, —OH, —OCF₃, —OCHF₂, —NH₂, alkyl, alkoxy, alkylamino, and cycloalkyl; provided that at least one of R₆ and R₇ is not —H; or

### Y₂ is

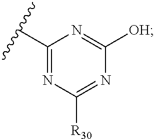

and
R₃₀ is selected from halogen, —CN, —OH, —OCF₃, —OCHF₂, —NH₂, alkyl, alkoxy, alkylamino, and cycloalkyl; or

### Y₂ is

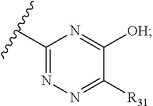

and
R₃₁ is selected from halogen, —CN, —OH, —OCF₃, —OCHF₂, —NH₂, alkyl, alkoxy, alkylamino, and cycloalkyl.

In certain embodiments, Y₂ is selected from

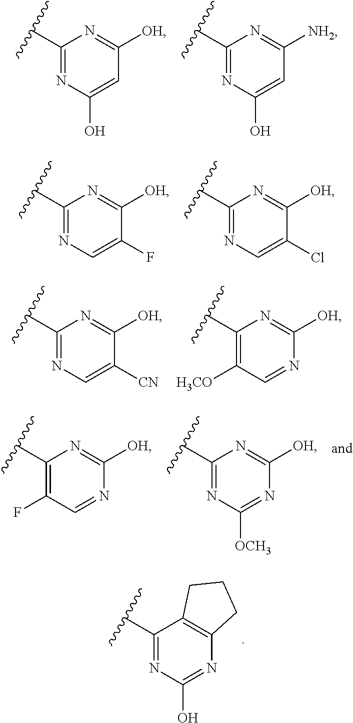

In certain embodiments, Y₂ is unsubstituted cycloalkyl or heterocyclyl.

In certain embodiments, Y₂ is selected from

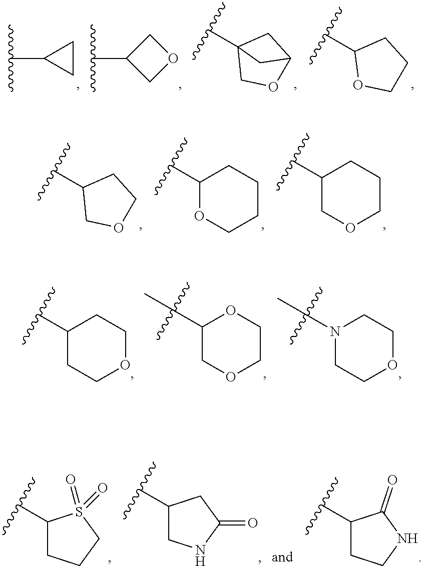

In certain embodiments, Y₂ is selected from

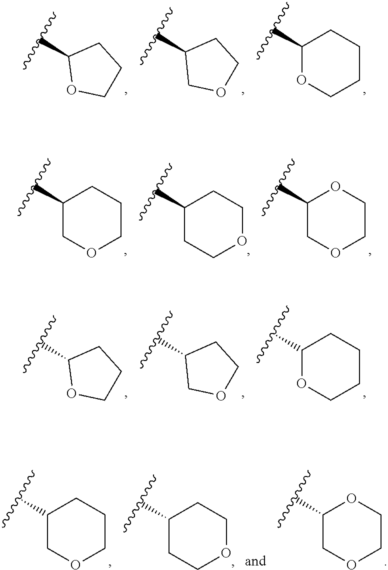

In certain embodiments, Y₂ is substituted cycloalkyl or heterocyclyl.

In certain embodiments, Y₂ is selected from

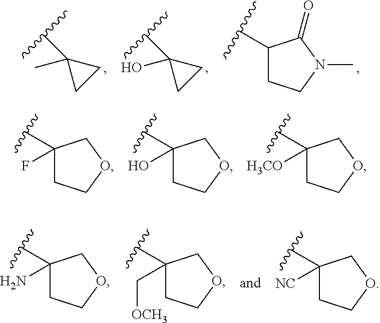

In certain embodiments, Y₂ is selected from

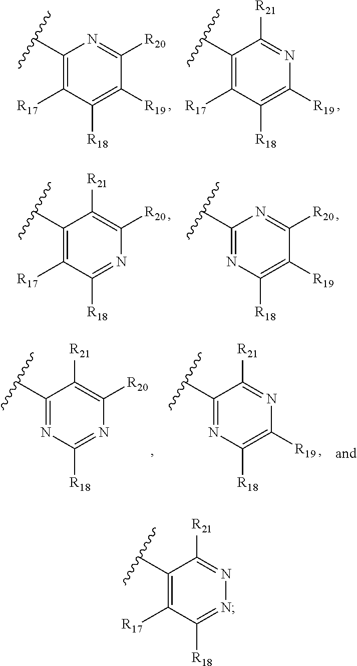

each occurrence of R₁₇, R₁₈, R₁₉, R₂₀, and R₂₁ is independently selected from —H, halogen, —CN, —NH₂, —OCF₃, —OCHF₂, —OAc, —NHAc, alkyl, haloalkyl, alkenyl, alkynyl, alkoxy, alkylamino cycloalkyl, aryl, heteroaryl, —C(O)NR₁₃R₁₄, and —CO₂R₁₅; and
each occurrence of R₁₃, R₁₄, and R₁₅ is independently selected from —H, alkyl, aryl, and heteroaryl.

In certain embodiments, at least one of R₁₇, R₁₈, R₁₉, R₂₀, and R₂₁ is not —H.

In certain embodiments, Y₂ is selected from

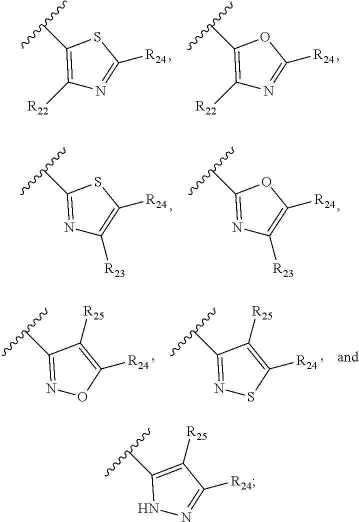

each occurrence of R₂₂, R₂₃, R₂₄, and R₂₅ is independently selected from —H, halogen, —CN, —NH₂, —OCF₃, —OCHF₂, —OAc, —NHAc, alkyl, haloalkyl, alkenyl, alkynyl, alkoxy, alkylamino cycloalkyl, aryl, heteroaryl, —C(O)NR₁₃R₁₄, and —CO₂R₁₅; and
each occurrence of R₁₃, R₁₄, and R₁₅ is independently selected from —H, alkyl, aryl, and heteroaryl.

In certain embodiments, each occurrence of R₂₂, R₂₃, R₂₄, and R₂₅ is independently selected from —H, and —CH₃.

In certain embodiments, Y₂ is —NH(Y₂′).

In certain embodiments, Y₂′ is selected from —H, —OH, alkyl, alkoxy, alkoxyalkyl, and cycloalkyl.

In certain embodiments, Y₂′ is selected from —H, —OH, —OCH₃, —CH₃, —CH₂CH₂OCH₃, and

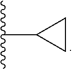 [SMILES: *C1CC1]

In certain embodiments, Y₂′ is selected from —H, alkyl, alkoxy, haloalkyl, and hydroxyalkyl.

In certain embodiments, Y₂′ is selected from —H, alkyl, alkoxy, and hydroxyalkyl.

In certain embodiments, Y₂′ is selected from —H, —OCH₃, —CH₃, —CH₂CH₃, —CH₂OH, —CH₂CH₂OH, —CH₂CH₂CH₂OH, —CH₂CH₂F, and —CH₂CH₂CH₂F.

In certain embodiments, Y₂′ is selected from —H, —OCH₃, —CH₃, —CH₂OH, and —CH₂CH₂OH.

In certain embodiments, Y₂ is —N(Y₂″)₂.

In certain embodiments, each Y₂″ is —CH₃.

In certain embodiments, both instances of Y₂″ taken together with the nitrogen atom to which they are bonded form a morpholinyl.

In certain embodiments, both instances of Y₂″ taken together with the nitrogen atom to which they are bonded form an azetidinyl.

In certain embodiments, Y₂′ is selected from cyanoalkyl, —O-alkoxyalkyl, —O— haloalkyl, and —O-hydroxyalkyl,

In certain embodiments, Y₂′ is selected from —CH₂CH₂CN, and —OCH₂CH₂CH₂CN, —OCH₂CHF₂, —OCH₂CH₂CHF₂, —CH₂CH₂OH, —CH₂CH₂OCH₃, and —OCH₂CH₂CH₂OH.

### Formulas (III) and (IV)

Also provided herein is a compound having the structure of Formula (III):

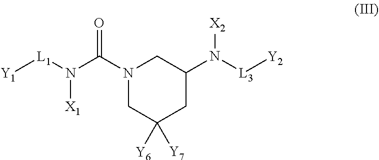

wherein:
L₁ is absent or selected from -alkyl-, -hydroxyalkyl-, -cycloalkyl-, and -heteroaryl-CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl; and
Y₆ and Y₇ together with the carbon to which they are bonded form a 4-, 5-, or 6-membered cycloalkyl or heterocyclyl;
or a pharmaceutically acceptable salt thereof.

In certain embodiments, Y₆ and Y₇ together with the carbon to which they are bonded form an unsubstituted 4-, 5-, or 6-membered heterocyclyl.

In certain embodiments, Y₆ and Y₇ together with the carbon to which they are bonded form an unsubstituted 4-, 5-, or 6-membered cyclic urea, cyclic carbamate, cyclic sulfone, cyclic sulfonamide, lactam, azalactam, or lactone.

In certain embodiments, Y₆ and Y₇ together with the carbon to which they are bonded form any one of the following structures:

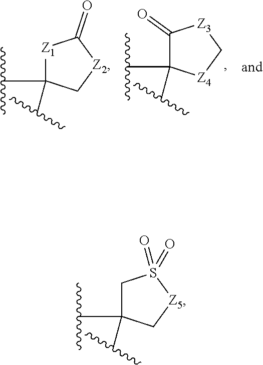

wherein
Z₁ is selected from O, NH, and CH₂;
Z₂ is selected from O, NH, and CH₂;
Z₃ is selected from O and NH;
Z₄ is selected from NH and CH₂; and
Z₅ is selected from NH and CH₂;
provided that one of Z₁ and Z₂ is not CH₂.

In certain embodiments, Y₆ and Y₇ together with the carbon to which they are bonded form the following structure:

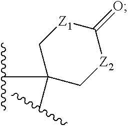

wherein Z₁ is selected from O, NH, and CH₂; and Z₂ is selected from O, NH, and CH₂; provided that one of Z₁ and Z₂ is not CH₂.

In certain embodiments, Y₆ and Y₇ together with the carbon to which they are bonded form any one of the following structures:

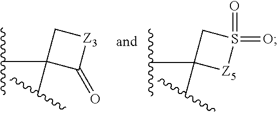

wherein Z₃ is selected from O and NH; and Z₅ is selected from NH and CH₂.

In certain embodiments, Y₆ and Y₇ together with the carbon to which they are bonded form an unsubstituted 4-, 5-, or 6-membered cycloheteroalkyl.

In certain embodiments, Y₆ and Y₇ together with the carbon to which they are bonded form an unsubstituted piperidinyl, tetrahydrofuranyl, azetidinyl, or morpholinyl.

In certain embodiments, Y₆ and Y₇ together with the carbon to which they are bonded form any one of the following structures:

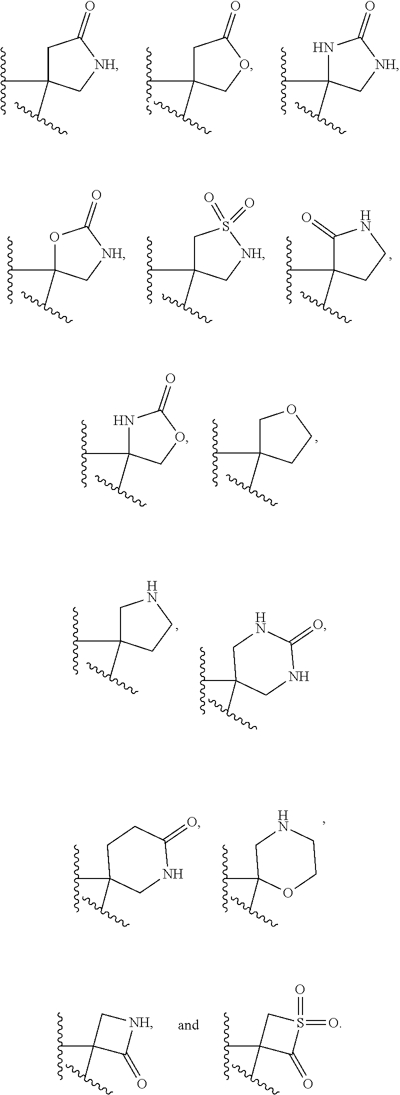

In certain embodiments, Y₆ and Y₇ together with the carbon to which they are bonded form a substituted 4-, 5-, or 6-membered heterocyclyl.

In certain embodiments, Y₆ and Y₇ together with the carbon to which they are bonded form a substituted 4-, 5-, or 6-membered cyclic urea, cyclic carbamate, cyclic sulfone, cyclic sulfonamide, lactam, azalactam, or lactone.

In certain embodiments, the cyclic urea, cyclic carbamate, cyclic sulfonamide, lactam, or azalactam is N-substituted.

In certain embodiments, cyclic urea, cyclic carbamate, cyclic sulfonamide, lactam, or azalactam is N-alkyl substituted.

In certain embodiments, Y₆ and Y₇ together with the carbon to which they are bonded form the following structure:

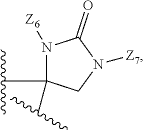

wherein Z₆ is selected from —H and alkyl; and Z₇ is selected from —H and alkyl;
provided that Z and Z₇ are not both —H.

In certain embodiments, Y₆ and Y₇ together with the carbon to which they are bonded form any one of the following structures:

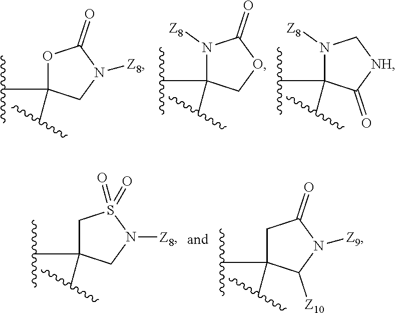

wherein each Z₈ is independently an alkyl; Z₉ is selected from —H and alkyl; and Z₁₀ is selected from —H and alkyl; provided that Z₉ and Z₁₀ are not both —H.

In certain embodiments, Y₆ and Y₇ together with the carbon to which they are bonded form any one of the following structures:

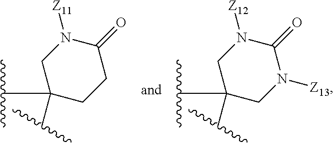

wherein Z₁₁ is alkyl; Z₁₂ is selected from —H and alkyl; and Z₁₃ is selected from —H and alkyl; provided that Z₁₂ and Z₁₃ are not both —H.

In certain embodiments, Y₆ and Y₇ together with the carbon to which they are bonded form the following structure:

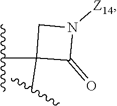

wherein Z₁₄ is alkyl.

In certain embodiments, Y₆ and Y₇ together with the carbon to which they are bonded form a substituted 4-, 5-, or 6-membered cycloheteroalkyl.

In certain embodiments, Y₆ and Y₇ together with the carbon to which they are bonded form a substituted piperidinyl, tetrahydrofuranyl, azetidinyl, or morpholinyl.

In certain embodiments, Y₆ and Y₇ together with the carbon to which they are bonded form an N-alkyl or N-acetyl substituted piperidinyl, azetidinyl, or morpholinyl.

In certain embodiments, Y₆ and Y₇ together with the carbon to which they are bonded form a substituted 4-, 5-, or 6-membered cycloalkyl.

In certain embodiments, Y₆ and Y₇ together with the carbon to which they are bonded form a substituted cyclobutyl.

In certain embodiments, Y₆ and Y₇ together with the carbon to which they are bonded form any one of the following structures:

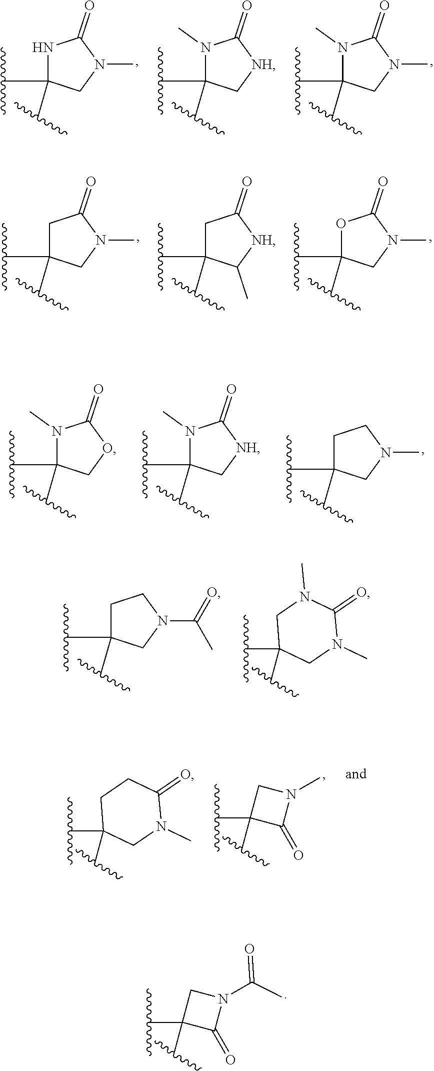

In certain embodiments, the compound having the structure selected from:

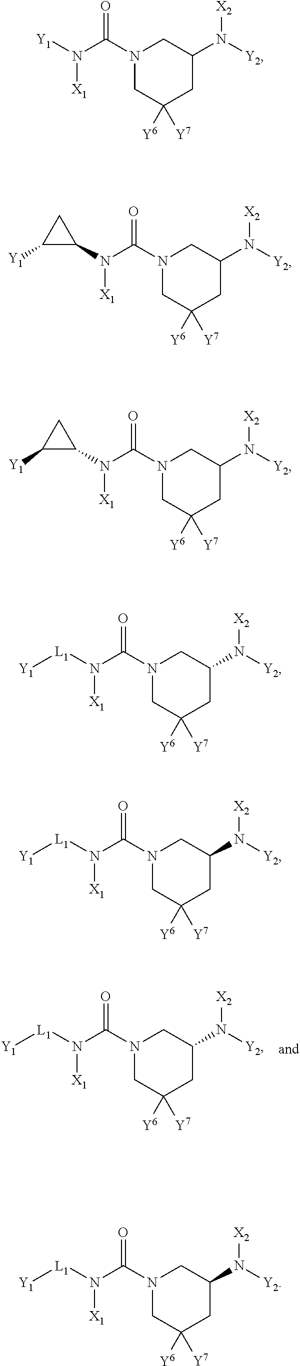

Also provided herein is a compound having the structure of Formula (IV):

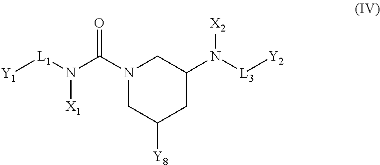

wherein:
L₁ is absent or selected from -alkyl-, -hydroxyalkyl-, -cycloalkyl-, -heteroaryl-, and -heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl;
Y₈ is selected from cyano, cycloalkyl, heteroaryl, heterocyclyl, alkyl-Y₈′, and a squaramide moiety;
Y₈′ is selected from —CN, —OH, —NH₂, —NH(Y₈″), —C(O)N(Y₈′″)₂, —SO₂N(Y₈′″)₂, and a squaramide moiety;
Y₈″ is selected from alkyl, —C(O)-alkyl, and —SO₂-alkyl; and
Y₈′″ is independently for each occurrence selected from —H and alkyl; or a pharmaceutically acceptable salt thereof.

In certain embodiments, the compound having the structure:

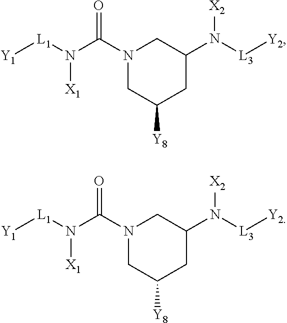

In certain embodiments, Y₈ is an unsubstituted 5-membered heteroaryl.

In certain embodiments, Y₈ is selected from an unsubstituted pyrazolyl, unsubstituted diazolyl, unsubstituted oxazolyl, and unsubstituted isooxazolyl.

In certain embodiments, Y₇ is selected from

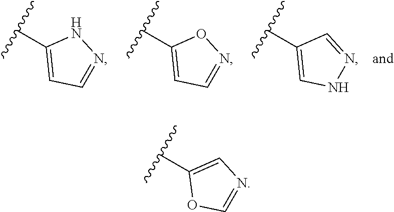 [SMILES: *c1ccn[nH]1 ; *c1ccno1 ; *c1cn[nH]c1 ; "and" ; *c1cnco1]

In certain embodiments, Y₈ is a substituted 6-membered heteroaryl.

In certain embodiments, Y₈ is selected from substituted pyridinyl and substituted pyrimidinyl.

In certain embodiments, Y₈ is selected from

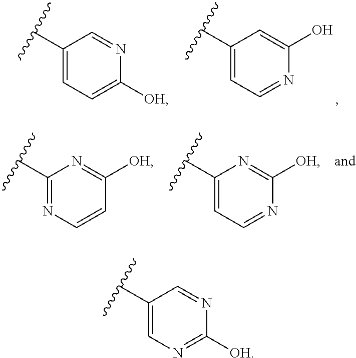 [SMILES: *c1ccc(O)nc1 ; *c1ccnc(O)c1 ; *c1nccc(O)n1 ; *c1ccnc(O)n1 ; "and" ; *c1cnc(O)nc1]

In certain embodiments, Y₈ is alkyl-Y₈′.

In certain embodiments, Y₈ is C₁-C₄ alkyl-Y₈′; and the alkyl is unbranched.

In certain embodiments, Y₈ is C₁-C₄ alkyl-Y₈′; and the alkyl is branched.

In certain embodiments, Y₈ is C₁-C₄ alkyl-Y₈′; and the alkyl is substituted with a cycloalkyl.

In certain embodiments, Y₈′ is selected from —NH(Y₈″), —C(O)N(Y₅′″)₂, and —SO₂N(Y₅′″)₂; Y₅″ is selected from —C(O)—CH₃, and —SO₂—CH₃; and Y₅′″ is independently for each occurrence selected from —H and —CH₃.

In certain embodiments, Y₈′ is a squaramide moiety.

In certain embodiments, Y₈′ is

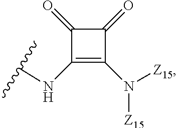

and Z₁₅ is independently for each occurrence selected from —H and alkyl.

In certain embodiments, each Z₁₅ is —H, each Z₁₅ is —CH₃, or one Z₁₅ is —H and the other is —CH₃.

In certain embodiments, Y₅ is a squaramide moiety.

In certain embodiments, Y₈ is

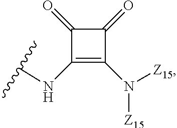

and Z₁₅ is independently for each occurrence selected from —H and alkyl.

In certain embodiments, each Z₁₅ is —H, each Z₁₅ is —CH₃, or one Z₁₅ is —H and the other is —CH₃.

In certain embodiments, Y₈ is selected from

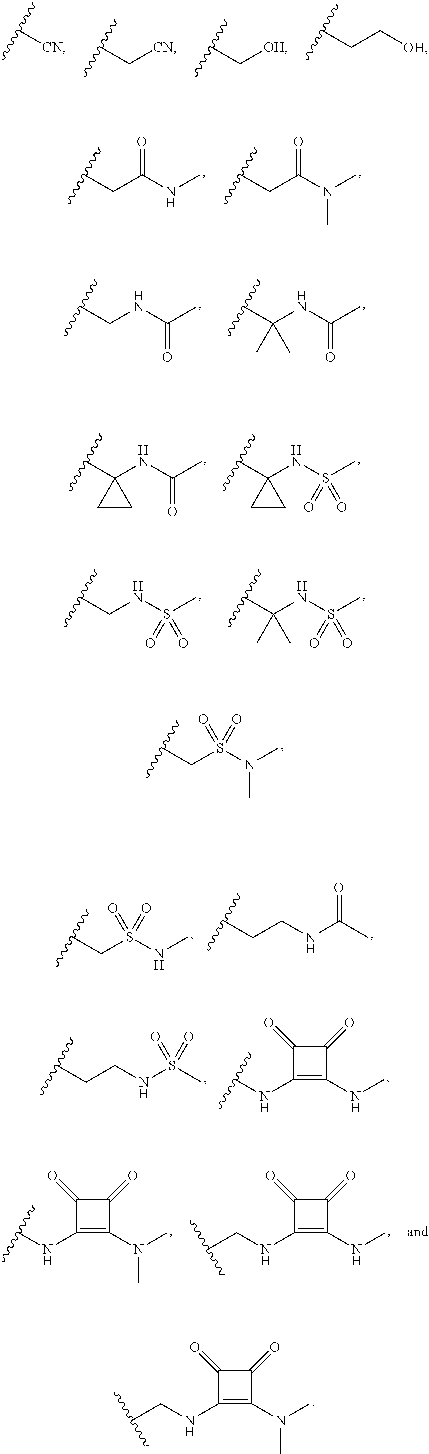

In certain embodiments, the compound having the structure selected from:

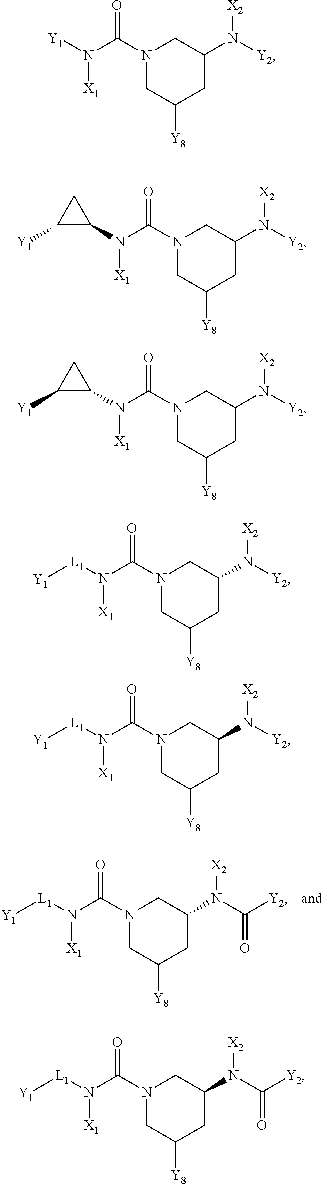

### Further Embodiments of Formulas (III) and (IV)

In certain embodiments, one of X₁ and X₂ is —H; and the other of X₁ and X₂ is selected from —CH₃, —CH₂CH₃, —CH₂CF₃, —CH₂CH₂CH₃,

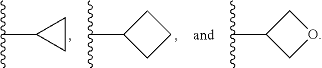

In certain embodiments, X₁ is —H; and X₂ is

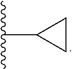 [SMILES: *C1CC1]

In certain embodiments, X₁ is —H; and X₂ is —CH₃. In other embodiments, X₁ is —H; and X₂ is —CH₂CH₃. In other embodiments, X₁ is —H; and X₂ is —CH₂CH₂CH₃.

In certain embodiments, X₂ is —H; and X₁ is

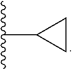 [SMILES: *C1CC1]

In certain embodiments, X₂ is —H; and X₁ is —CH₃. In other embodiments, X₂ is —H; and X₁ is —CH₂CH₃. In other embodiments, X₂ is —H; and X₁ is —CH₂CH₂CH₃.

In certain embodiments, L₁ is absent.

In certain embodiments, L₁ is selected from -alkyl-, -hydroxyalkyl-, -cycloalkyl-, -heteroaryl-, and -heteroaryl-CH₂—.

In certain embodiments, L₁ is selected from —CH₂—, —C(H)(CH₃)—, —CH₂CH₂—, and —C(H)(OH)CH₂—.

In certain embodiments, L₁ is

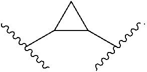 [SMILES: *C1CC1*]

In certain embodiments, L₁ is selected from

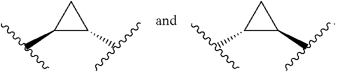 [SMILES: *[C@H]1C[C@@H]1* ; *[C@@H]1C[C@H]1*]

In certain embodiments, L₁ is selected from

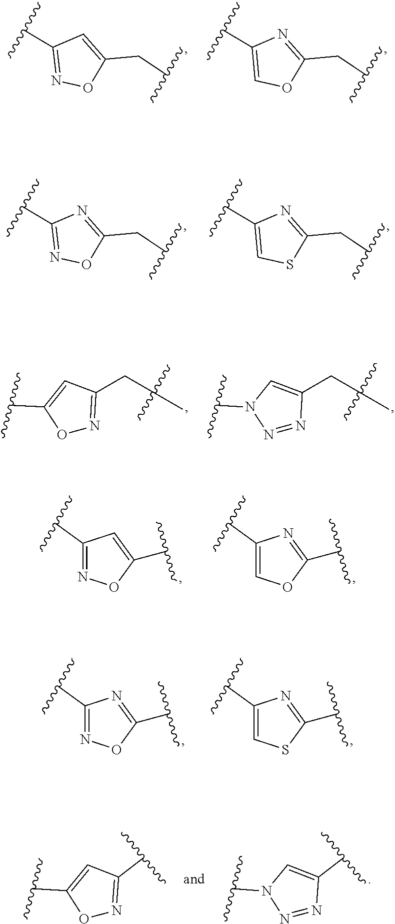

In certain embodiments, Y₁ is unsubstituted aryl. In other embodiments, Y₁ is selected from unsubstituted phenyl and unsubstituted naphthyl.

In certain embodiments, Y₁ is substituted aryl.

In certain embodiments, Y₁ is

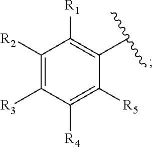

and
R₁, R₂, R₃, R₄, and R₅ are independently selected from —H, halogen, —CN, —CF₃, —CHF₂, —CF₂CH₃, —OCF₃, —OCHF₂, alkyl, haloalkyl, alkenyl, alkynyl, alkoxy, cycloalkyl, heterocyclyl, aryl, and heteroaryl; provided that one of R₁, R₂, R₃, R₄, and R₅ is not —H.

In certain embodiments, R₁, R₂, R₃, R₄, and R₅ are independently selected from —H, —F, —Cl, —Br, —CN, —CH₃, —CH₂CH₃, —CF₃, —CHF₂, —CF₂CH₃, —OCH₃, —OCF₃, —OCHF₂,

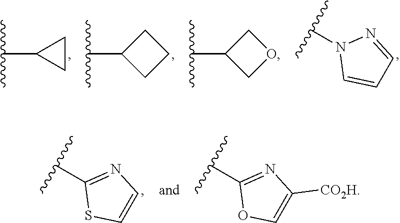

In certain embodiments, R₁, R₂, R₃, R₄, and R₅ are independently selected from —H, —F, —Cl, —Br, —CN, —CH₃, —CH₂CH₃, —CH₂CH₂CH₃, —CH(CH₃)₂, —OCH₃, —OCF₃, and

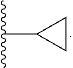 [SMILES: *C1CC1]

In certain embodiments, two of R₁, R₂, R₃, R₄, and R₅ are not —H, or three of R₁, R₂, R₃, R₄, and R₅ are not —H.

In certain embodiments, Y₁ is selected from

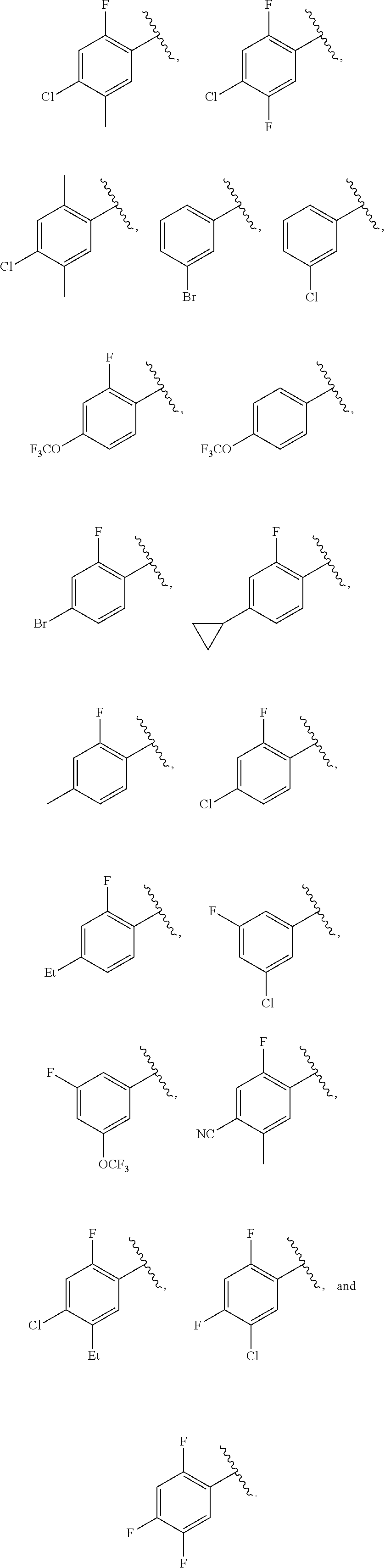

In certain embodiments, Y₁ is

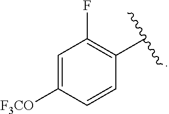 [SMILES: *c1ccc(OC(F)(F)F)cc1F]

In certain embodiments, Y₁ is unsubstituted heteroaryl.

In certain embodiments, Y₁ is selected from

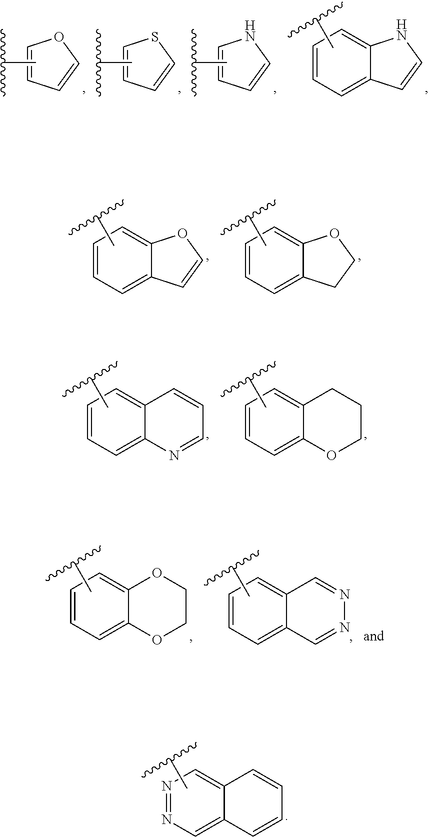

In certain embodiments, Y₁ is substituted heteroaryl.

In certain embodiments, Y₁ is selected from

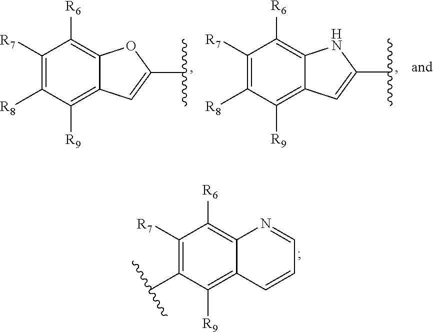

and
each occurrence of R₆, R₇, R₈, and R₉ are independently selected from —H, halogen, —CN, —OCF₃, —OCHF₂, alkyl, haloalkyl, alkenyl, alkynyl, alkoxy, cycloalkyl, aryl, and heteroaryl; provided that at least one of R₆, R₇, R₈, and R₉ is not —H.

In certain embodiments, L₃ is absent.

In certain embodiments, Y₂ is unsubstituted heteroaryl.

In certain embodiments, Y₂ is selected from

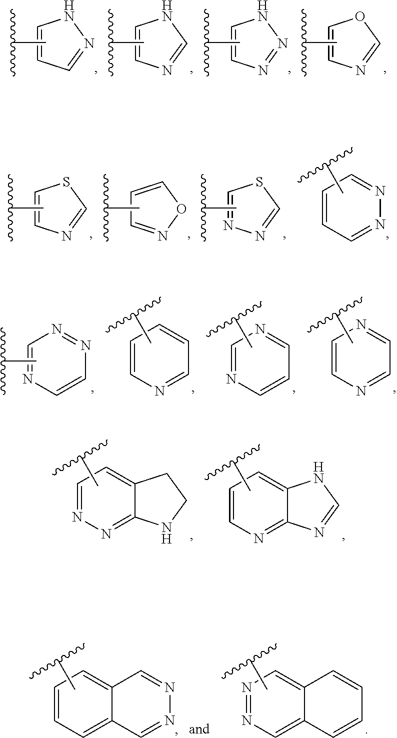

In certain embodiments, Y₂ is

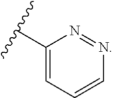 [SMILES: *c1cccnn1]

In certain embodiments, Y₂ is substituted heteroaryl.

In certain embodiments, Y₂ is

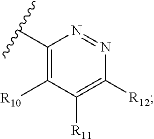

R₁₀, R₁₁, and R₁₂ are independently selected from —H, halogen, —CN, —OH, —NH₂, —OCF₃, —OCHF₂, —OAc, —NHAc, alkyl, haloalkyl, hydroxyalkyl, alkenyl, alkynyl, alkoxy, alkylamino cycloalkyl, aryl, heteroaryl, —C(O)NR₁₃R₁₄, —CO₂R₁₅, and —C(O)NHSO₂R₁₅; provided that at least one of R₁₀, R₁₁, and R₁₂ is not —H; and
each occurrence of R₁₃, R₁₄, and R₁₅ is independently selected from —H, alkyl, aryl, and heteroaryl.

In certain embodiments, R₁₀, R₁₁, and R₁₂ are independently selected from —H, —F, —Cl, —Br, —CN, —CH₃, —CH₂CH₃, —CF₃, —CHF₂, —CF₂CH₃, —OCH₃, —OCF₃, —OCHF₂, —OAc, —NH₂, —NHCH₃, —NHAc, —C(O)NH₂, —C(O)NHCH₃, —C(O)NHCH₂CH₃, —C(O)NHSO₂CH₃, —C(O)NHSO₂CH₂CH₃, —CH₂OH, —CO₂H, phenyl, cyclopropyl, cyclobutyl, imidazolyl, and tetrazolyl.

In certain embodiments, R₁₀ and R₁₂ are each —H; and R₁₁ is selected from —CN, —CF₃, —CH₃, —OCH₃, —NH₂, —NHCH₃, —NHAc, —CO₂H, —C(O)NH₂, —C(O)NHCH₃, —C(O)NHCH₂CH₃,

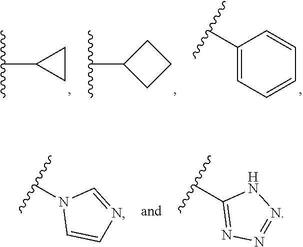

In certain embodiments, R₁₁ and R₁₂ are each —H; and R₁₀ is selected from —CN, —CF₃, —CH₃, —OCH₃, —NH₂, —NHCH₃, —NHAc, —CO₂H, —C(O)NH₂, —C(O)NHCH₃, —C(O)NHCH₂CH₃,

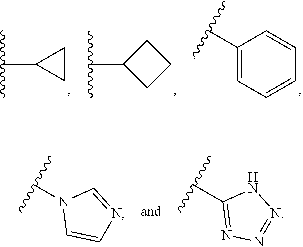

In certain embodiments, R₁₀ and R₁₁ are each —H; and R₁₂ is selected from —CN, —CF₃, —CH₃, —OCH₃, —NH₂, —NHCH₃, —NHAc, —CO₂H, —C(O)NH₂, —C(O)NHCH₃, —C(O)NHCH₂CH₃,

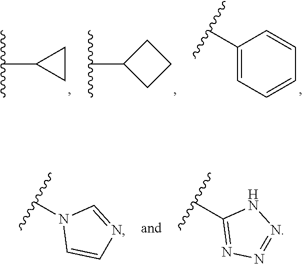

In certain embodiments, Y₂ is selected from

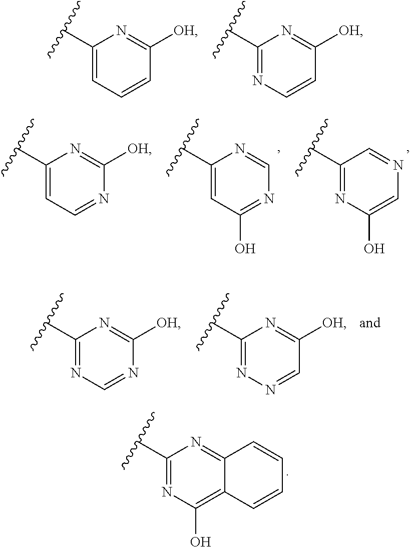

In certain embodiments, Y₂ is

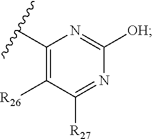

and
R₂₆ and R₂₇ are independently selected from —H, halogen, —CN, —OH, —OCF₃, —OCHF₂, —NH₂, alkyl, alkoxy, alkylamino, and cycloalkyl; provided that at least one of R₆ and R₇ is not —H; or R₆ and R₇ taken together with the carbon atoms to which they are bonded form an unsubstituted or substituted fused C₅-C₇ cycloalkyl; or

### Y₂ is

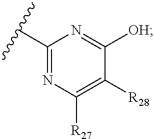

and
R₂₇ and R₂₈ are independently selected from —H, halogen, —CN, —OH, —OCF₃, —OCHF₂, —NH₂, alkyl, alkoxy, alkylamino, and cycloalkyl; provided that at least one of R₇ and R₈ is not —H; or R₇ and R₈ taken together with the carbon atoms to which they are bonded form an unsubstituted or substituted fused C₅-C₇ cycloalkyl; or

### Y₂ is

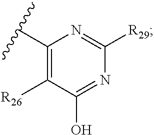

and
R₂₆ and R₂₉ are independently selected from —H, halogen, —CN, —OH, —OCF₃, —OCHF₂, —NH₂, alkyl, alkoxy, alkylamino, and cycloalkyl; provided that at least one of R₆ and R₉ is not —H; or

### Y₂ is

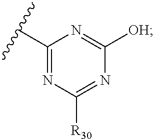

and
R₃₀ is selected from halogen, —CN, —OH, —OCF₃, —OCHF₂, —NH₂, alkyl, alkoxy, alkylamino, and cycloalkyl; or

### Y₂ is

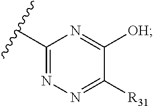

and
R₃₁ is selected from halogen, —CN, —OH, —OCF₃, —OCHF₂, —NH₂, alkyl, alkoxy, alkylamino, and cycloalkyl.

In certain embodiments, Y₂ is selected from

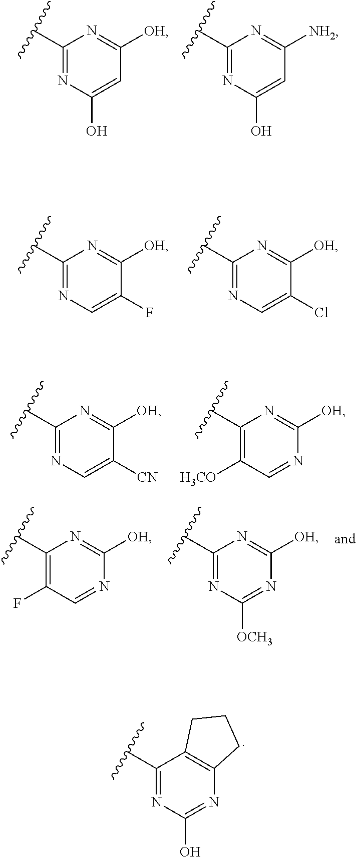

In certain embodiments, Y₂ is selected from

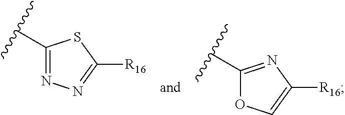

R₁₆ for each occurrence is independently selected from halogen, —CN, —NH₂, —OCF₃, —OCHF₂, —OAc, —NHAc, alkyl, haloalkyl, alkenyl, alkynyl, alkoxy, alkylamino cycloalkyl, aryl, heteroaryl, —C(O)NR₁₃R₁₄, and —CO₂R₁₅; and
each occurrence of R₁₃, R₁₄, and R₁₅ is independently selected from —H, alkyl, aryl, and heteroaryl.

In certain embodiments, R₁₆ is selected from —CN, —CH₃, —CF₃, —C(O)NH₂, —CO₂CH₂CH₃, and

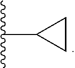 [SMILES: *C1CC1]

In certain embodiments, Y₂ is selected from

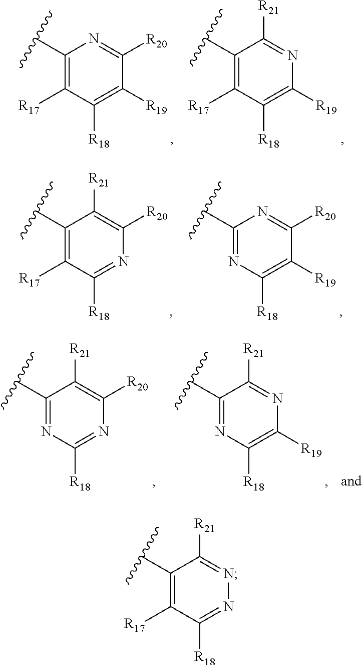

each occurrence of R₁₇, R₁₈, R₁₉, R₂₀, and R₂₁ is independently selected from —H, halogen, —CN, —NH₂, —OCF₃, —OCHF₂, —OAc, —NHAc, alkyl, haloalkyl, alkenyl, alkynyl, alkoxy, alkylamino cycloalkyl, aryl, heteroaryl, —C(O)NR₁₃R₁₄, and —CO₂R₁₅; provided that at least one of R₁₇, R₁₈, R₁₉, R₂₀, and R₂₁ is not —H; and
each occurrence of R₁₃, R₁₄, and R₁₅ is independently selected from —H, alkyl, aryl, and heteroaryl.

In certain embodiments, R₁₇, R₁₈, R₁₉, R₂₀, and R₂₁ are independently selected from —H, —CN, —CH₃, and —OCH₃.

In certain embodiments, Y₂ is selected from

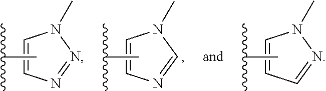

In certain embodiments, L₃ is —C(O)—.

In certain embodiments, Y₂ is selected from alkyl, alkenyl, alkynyl, alkoxy, alkoxyalkyl, hydroxyalkyl, and haloalkyl.

In certain embodiments, Y₂ is selected from —CH₃, —CF₃, —CH₂CH(CH₃)₂, —CH₂CH₂C≡CH, —CH₂CH₂OCH₃, —C(H)(CH₃)CH₂OCH₃, —OCH₃, —CH₂OH, —CH₂CH₂OH, —C(CH₃)₂OH, and —CH₂OCH₃.

In certain embodiments, Y₂ is selected from —CH₂OH and —CH₂CH₂OH.

In certain embodiments, Y₂ is unsubstituted heteroaryl.

In certain embodiments, Y₂ is

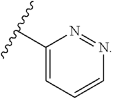 [SMILES: *c1cccnn1]

In certain embodiments, Y₂ is substituted heteroaryl.

In certain embodiments, Y₂ is selected from

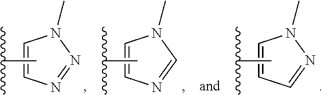

In certain embodiments, Y₂ is

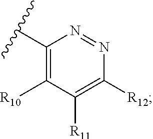

R₁₀, R₁₁, and R₁₂ are independently selected from —H, halogen, —CN, —OH, —NH₂, —OCF₃, —OCHF₂, —OAc, —NHAc, alkyl, haloalkyl, hydroxyalkyl, alkenyl, alkynyl, alkoxy, alkylamino cycloalkyl, aryl, heteroaryl, —C(O)NR₁₃R₁₄, and —CO₂R₁₅; provided that at least one of R₁₀, R₁₁, and R₁₂ is not —H; and
each occurrence of R₁₃, R₁₄, and R₁₅ is independently selected from —H, alkyl, aryl, and heteroaryl.

In certain embodiments, R₁₀, R₁₁, and R₁₂ are independently selected from —H, —F, —Cl, —Br, —CN, —CH₃, —CH₂CH₃, —CF₃, —CHF₂, —CF₂CH₃, —OCH₃, —OCF₃, —OCHF₂, —OAc, —NH₂, —NHCH₃, —NHAc, —C(O)NH₂, —C(O)NHCH₃, —C(O)NHCH₂CH₃, —C(O)NHSO₂CH₃, —C(O)NHSO₂CH₂CH₃, —CH₂OH, —CO₂H, phenyl, cyclopropyl, cyclobutyl, imidazolyl, and tetrazolyl.

In certain embodiments, Y₂ is

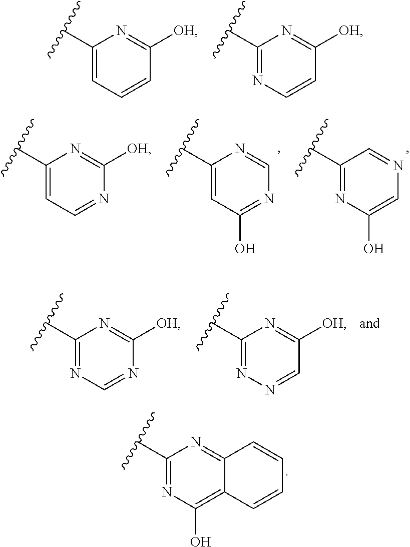

In certain embodiments, Y₂ is

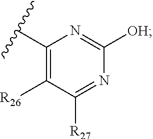

and
R₂₆ and R₂₇ are independently selected from —H, halogen, —CN, —OH, —OCF₃, —OCHF₂, —NH₂, alkyl, alkoxy, alkylamino, and cycloalkyl; provided that at least one of R₆ and R₇ is not —H; or R₆ and R₇ taken together with the carbon atoms to which they are bonded form an unsubstituted or substituted fused C₅-C₇ cycloalkyl; or

### Y₂ is

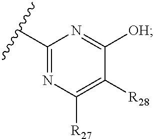

and
R₂₇ and R₂₈ are independently selected from —H, halogen, —CN, —OH, —OCF₃, —OCHF₂, —NH₂, alkyl, alkoxy, alkylamino, and cycloalkyl; provided that at least one of R₇ and R₈ is not —H; or R₇ and R₈ taken together with the carbon atoms to which they are bonded form an unsubstituted or substituted fused C₅-C₇ cycloalkyl; or

### Y₂ is

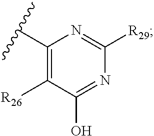

and
R₂₆ and R₂₉ are independently selected from —H, halogen, —CN, —OH, —OCF₃, —OCHF₂, —NH₂, alkyl, alkoxy, alkylamino, and cycloalkyl; provided that at least one of R₆ and R₉ is not —H; or

### Y₂ is

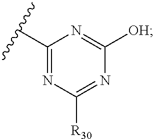

and
R₃₀ is selected from halogen, —CN, —OH, —OCF₃, —OCHF₂, —NH₂, alkyl, alkoxy, alkylamino, and cycloalkyl; or

### Y₂ is

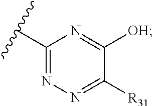

and
R₃₁ is selected from halogen, —CN, —OH, —OCF₃, —OCHF₂, —NH₂, alkyl, alkoxy, alkylamino, and cycloalkyl.

In certain embodiments, Y₂ is selected from

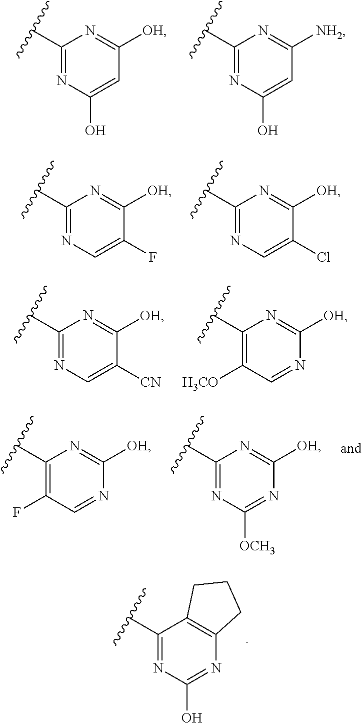

In certain embodiments, Y₂ is substituted cycloalkyl or heterocyclyl.

In certain embodiments, Y₂ is selected from

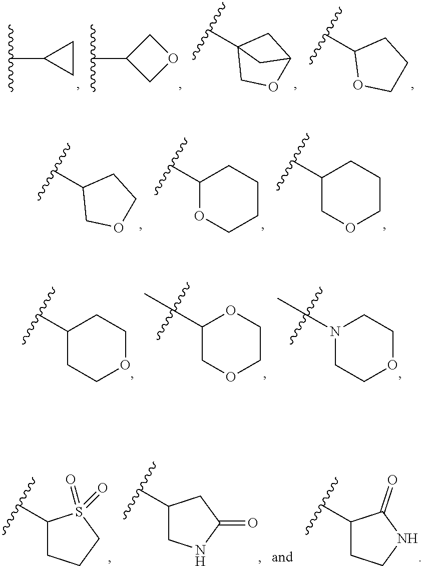

In certain embodiments, Y₂ is selected from

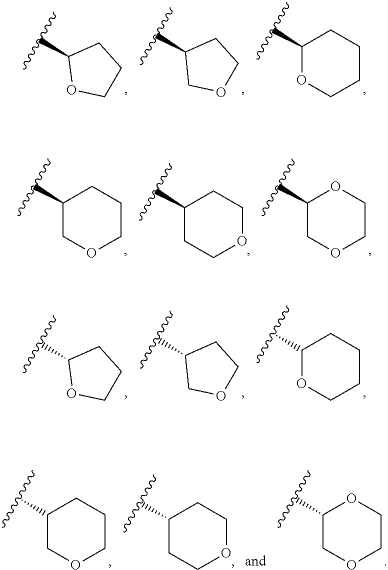

In certain embodiments, Y₂ is substituted cycloalkyl or heterocyclyl.

In certain embodiments, Y₂ is selected from

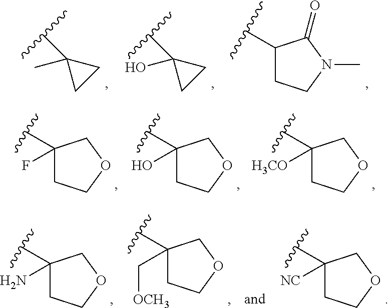

In certain embodiments, Y₂ is selected from

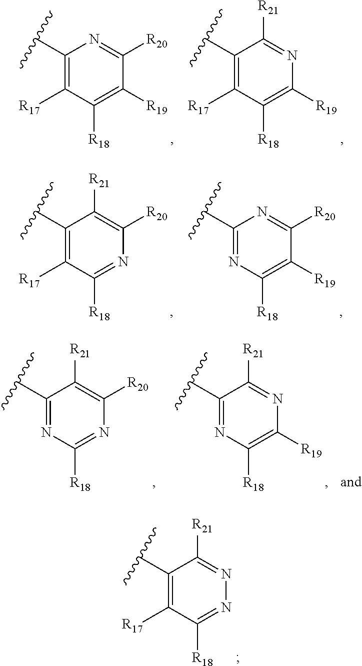

each occurrence of R₁₇, R₁₈, R₁₉, R₂₀, and R₂₁ is independently selected from —H, halogen, —CN, —NH₂, —OCF₃, —OCHF₂, —OAc, —NHAc, alkyl, haloalkyl, alkenyl, alkynyl, alkoxy, alkylamino cycloalkyl, aryl, heteroaryl, —C(O)NR₁₃R₁₄, and —CO₂R₁₅; and
each occurrence of R₁₃, R₁₄, and R₁₅ is independently selected from —H, alkyl, aryl, and heteroaryl.

In certain embodiments, at least one of R₁₇, R₁₈, R₁₉, R₂₀, and R₂₁ is not —H.

In certain embodiments, Y₂ is selected from

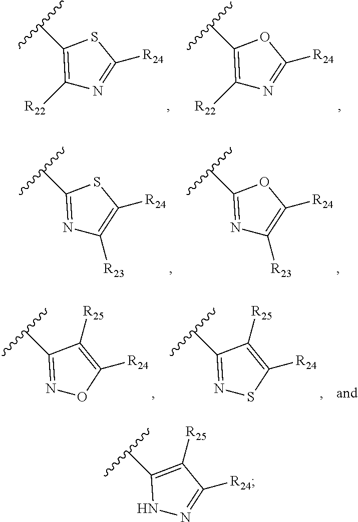

each occurrence of R₂₂, R₂₃, R₂₄, and R₂₅ is independently selected from —H, halogen, —CN, —NH₂, —OCF₃, —OCHF₂, —OAc, —NHAc, alkyl, haloalkyl, alkenyl, alkynyl, alkoxy, alkylamino cycloalkyl, aryl, heteroaryl, —C(O)NR₁₃R₁₄, and —CO₂R₁₅; and
each occurrence of R₁₃, R₁₄, and R₁₅ is independently selected from —H, alkyl, aryl, and heteroaryl.

In certain embodiments, each occurrence of R₂₂, R₂₃, R₂₄, and R₂₅ is independently selected from —H, and —CH₃.

In certain embodiments, Y₂ is —NH(Y₂′).

In certain embodiments, Y₂′ is selected from —H, —OH, alkyl, alkoxy, alkoxyalkyl, and cycloalkyl.

In certain embodiments, Y₂′ is selected from —H, —OH, —OCH₃, —CH₃, —CH₂CH₂OCH₃, and

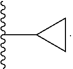 [SMILES: *C1CC1]

In certain embodiments, Y₂′ is selected from —H, alkyl, alkoxy, and hydroxyalkyl.

In certain embodiments, Y₂′ is selected from —H, —OCH₃, —CH₃, —CH₂OH, and —CH₂CH₂OH.

In certain embodiments, Y₂ is —N(Y₂″)₂.

In certain embodiments, each Y₂″ is —CH₃.

In certain embodiments, both Y₂″ taken together with the nitrogen atom to which they are bonded form a morpholinyl.

### Exemplary Compounds of Formula (I)

In certain embodiments, a compound or a pharmaceutically acceptable salt thereof having the structure of any one of the following compounds:

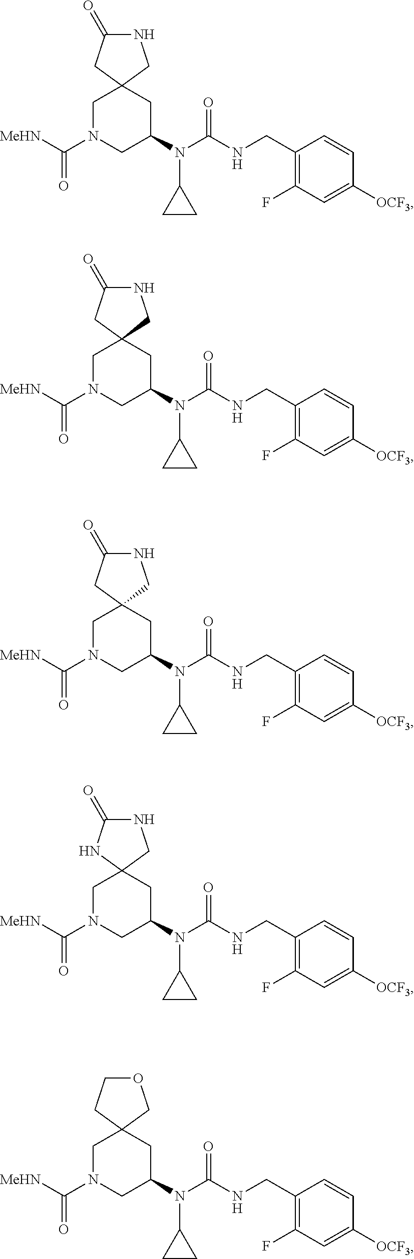 [SMILES: CNC(=O)N1C[C@H](N(C(=O)NCc2ccc(OC(F)(F)F)cc2F)C2CC2)CC2(CNC(=O)C2)C1 ; CNC(=O)N1C[C@H](N(C(=O)NCc2ccc(OC(F)(F)F)cc2F)C2CC2)C[C@]2(CNC(=O)C2)C1 ; CNC(=O)N1C[C@H](N(C(=O)NCc2ccc(OC(F)(F)F)cc2F)C2CC2)C[C@@]2(CNC(=O)C2)C1 ; CNC(=O)N1C[C@H](N(C(=O)NCc2ccc(OC(F)(F)F)cc2F)C2CC2)CC2(CNC(=O)N2)C1 ; CNC(=O)N1C[C@H](N(C(=O)NCc2ccc(OC(F)(F)F)cc2F)C2CC2)CC2(CCOC2)C1]

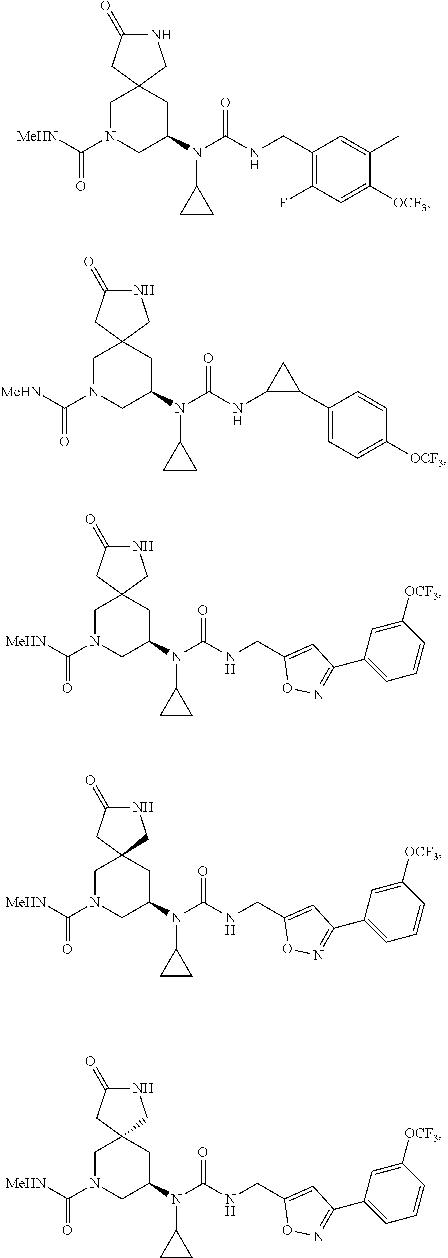 [SMILES: CNC(=O)N1C[C@H](N(C(=O)NCc2cc(C)c(OC(F)(F)F)cc2F)C2CC2)CC2(CNC(=O)C2)C1 ; CNC(=O)N1C[C@H](N(C(=O)NC2CC2c2ccc(OC(F)(F)F)cc2)C2CC2)CC2(CNC(=O)C2)C1 ; CNC(=O)N1C[C@H](N(C(=O)NCc2cc(-c3cccc(OC(F)(F)F)c3)no2)C2CC2)CC2(CNC(=O)C2)C1 ; CNC(=O)N1C[C@H](N(C(=O)NCc2cc(-c3cccc(OC(F)(F)F)c3)no2)C2CC2)C[C@]2(CNC(=O)C2)C1 ; CNC(=O)N1C[C@H](N(C(=O)NCc2cc(-c3cccc(OC(F)(F)F)c3)no2)C2CC2)C[C@@]2(CNC(=O)C2)C1]

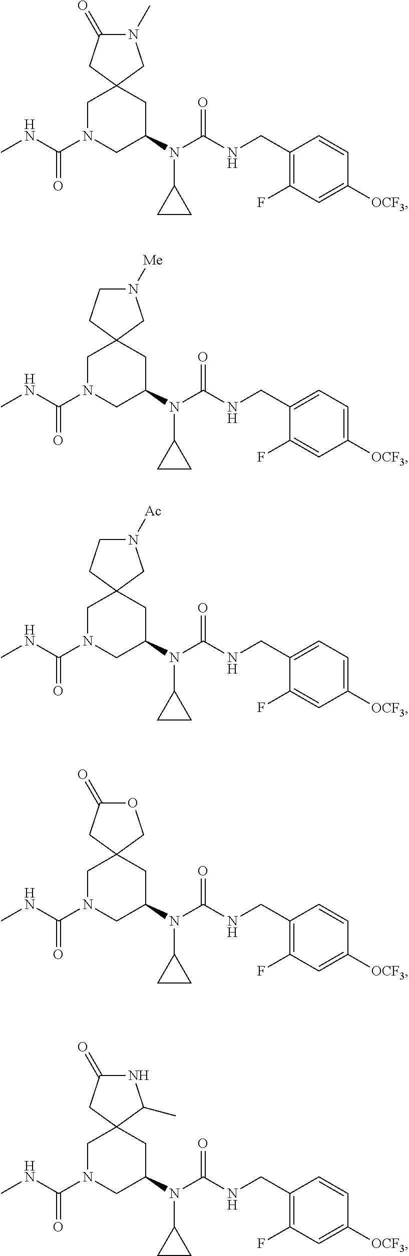

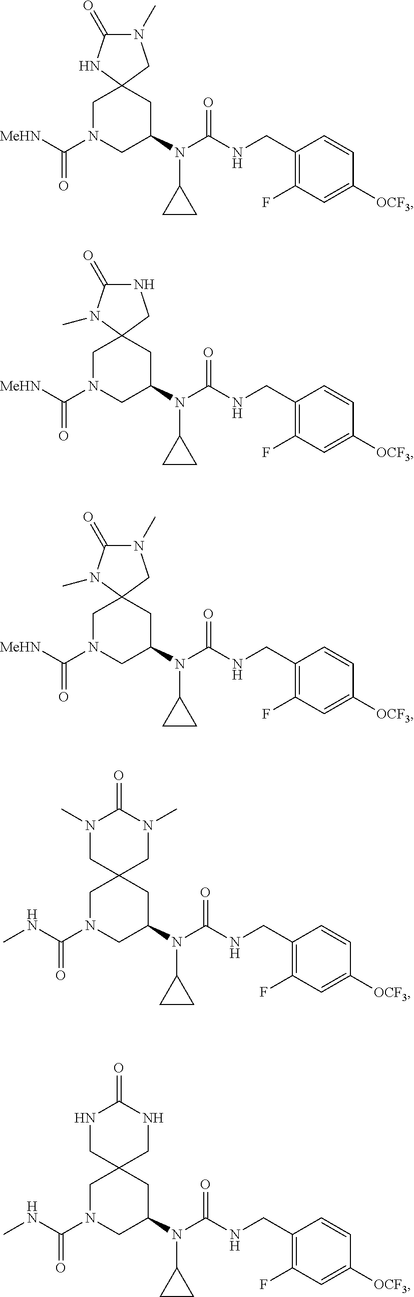 [SMILES: CNC(=O)N1C[C@H](N(C(=O)NCc2ccc(OC(F)(F)F)cc2F)C2CC2)CC2(CN(C)C(=O)N2)C1 ; CNC(=O)N1C[C@H](N(C(=O)NCc2ccc(OC(F)(F)F)cc2F)C2CC2)CC2(CNC(=O)N2C)C1 ; CNC(=O)N1C[C@H](N(C(=O)NCc2ccc(OC(F)(F)F)cc2F)C2CC2)CC2(CN(C)C(=O)N2C)C1 ; CNC(=O)N1C[C@H](N(C(=O)NCc2ccc(OC(F)(F)F)cc2F)C2CC2)CC2(CN(C)C(=O)N(C)C2)C1 ; CNC(=O)N1C[C@H](N(C(=O)NCc2ccc(OC(F)(F)F)cc2F)C2CC2)CC2(CNC(=O)NC2)C1]

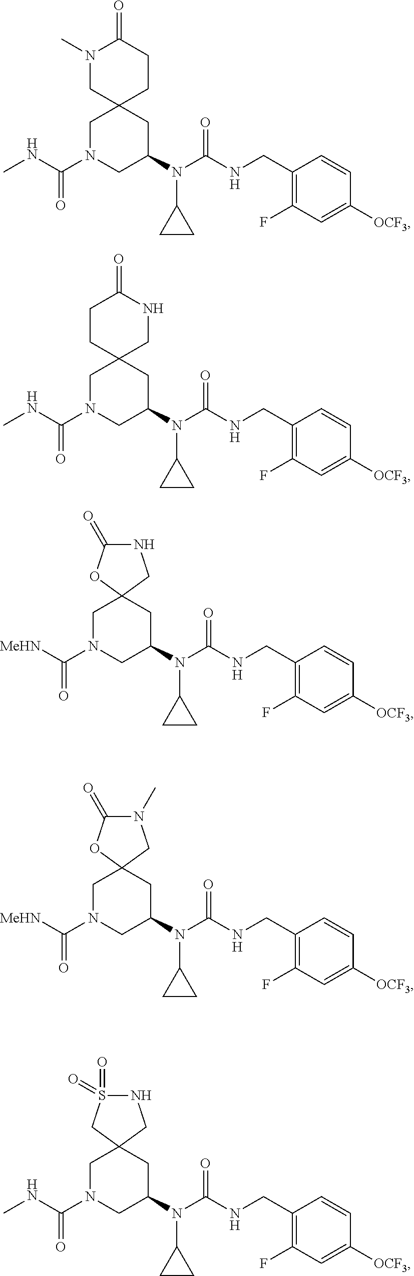 [SMILES: CNC(=O)N1C[C@H](N(C(=O)NCc2ccc(OC(F)(F)F)cc2F)C2CC2)CC2(CCC(=O)N(C)C2)C1 ; CNC(=O)N1C[C@H](N(C(=O)NCc2ccc(OC(F)(F)F)cc2F)C2CC2)CC2(CCC(=O)NC2)C1 ; CNC(=O)N1C[C@H](N(C(=O)NCc2ccc(OC(F)(F)F)cc2F)C2CC2)CC2(CNC(=O)O2)C1 ; CNC(=O)N1C[C@H](N(C(=O)NCc2ccc(OC(F)(F)F)cc2F)C2CC2)CC2(CN(C)C(=O)O2)C1 ; CNC(=O)N1C[C@H](N(C(=O)NCc2ccc(OC(F)(F)F)cc2F)C2CC2)CC2(CNS(=O)(=O)C2)C1]

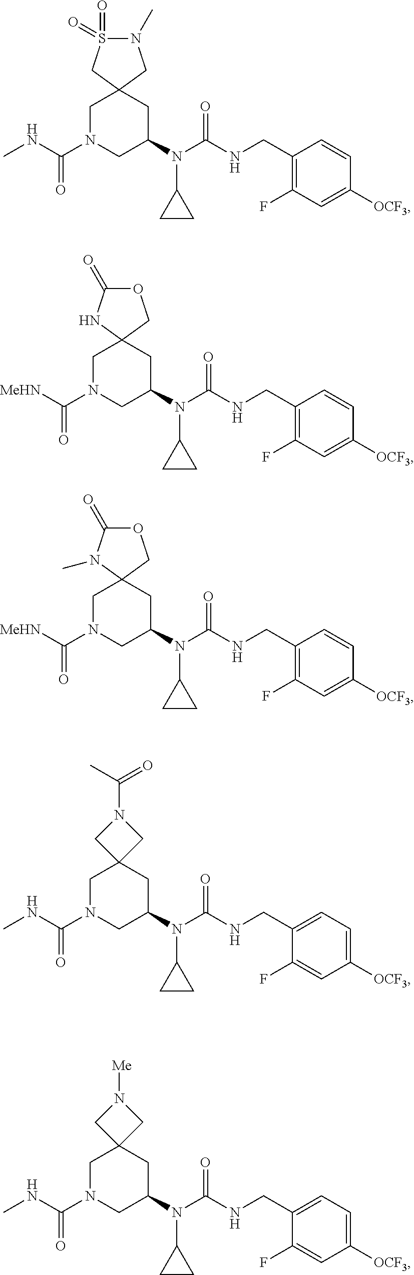 [SMILES: CNC(=O)N1C[C@H](N(C(=O)NCc2ccc(OC(F)(F)F)cc2F)C2CC2)CC2(C1)CN(C)S(=O)(=O)C2 ; CNC(=O)N1C[C@H](N(C(=O)NCc2ccc(OC(F)(F)F)cc2F)C2CC2)CC2(COC(=O)N2)C1 ; CNC(=O)N1C[C@H](N(C(=O)NCc2ccc(OC(F)(F)F)cc2F)C2CC2)CC2(COC(=O)N2C)C1 ; CNC(=O)N1C[C@H](N(C(=O)NCc2ccc(OC(F)(F)F)cc2F)C2CC2)CC2(CN(C(C)=O)C2)C1 ; CNC(=O)N1C[C@H](N(C(=O)NCc2ccc(OC(F)(F)F)cc2F)C2CC2)CC2(CN(C)C2)C1]

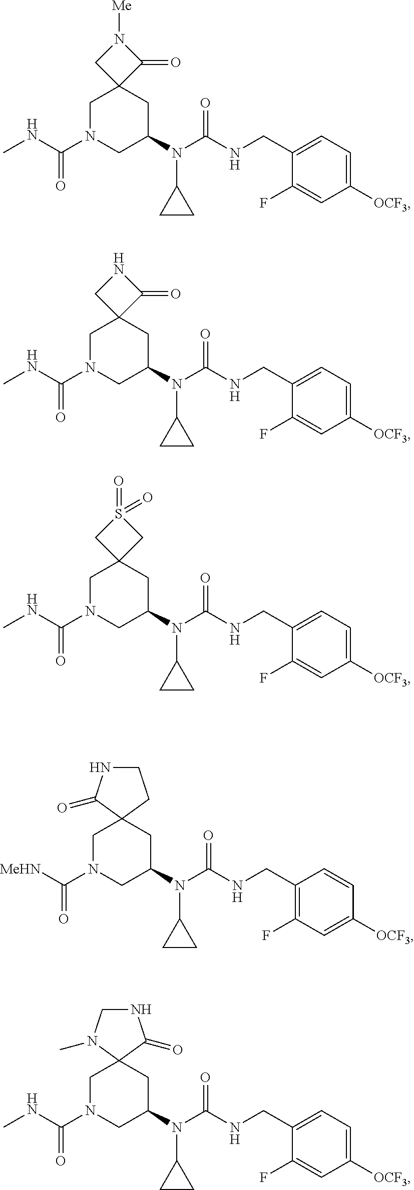 [SMILES: CNC(=O)N1C[C@H](N(C(=O)NCc2ccc(OC(F)(F)F)cc2F)C2CC2)CC2(CN(C)C2=O)C1 ; CNC(=O)N1C[C@H](N(C(=O)NCc2ccc(OC(F)(F)F)cc2F)C2CC2)CC2(CNC2=O)C1 ; CNC(=O)N1C[C@H](N(C(=O)NCc2ccc(OC(F)(F)F)cc2F)C2CC2)CC2(C1)CS(=O)(=O)C2 ; CNC(=O)N1C[C@H](N(C(=O)NCc2ccc(OC(F)(F)F)cc2F)C2CC2)CC2(CCNC2=O)C1 ; CNC(=O)N1C[C@H](N(C(=O)NCc2ccc(OC(F)(F)F)cc2F)C2CC2)CC2(C1)C(=O)NCN2C]

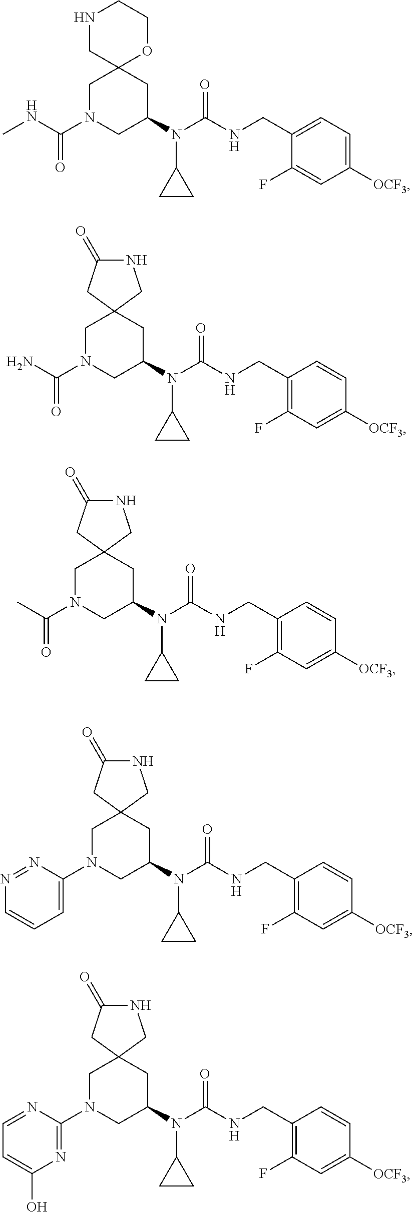 [SMILES: CNC(=O)N1C[C@H](N(C(=O)NCc2ccc(OC(F)(F)F)cc2F)C2CC2)CC2(CNCCO2)C1 ; NC(=O)N1C[C@H](N(C(=O)NCc2ccc(OC(F)(F)F)cc2F)C2CC2)CC2(CNC(=O)C2)C1 ; CC(=O)N1C[C@H](N(C(=O)NCc2ccc(OC(F)(F)F)cc2F)C2CC2)CC2(CNC(=O)C2)C1 ; O=C1CC2(CN1)C[C@@H](N(C(=O)NCc1ccc(OC(F)(F)F)cc1F)C1CC1)CN(c1cccnn1)C2 ; O=C1CC2(CN1)C[C@@H](N(C(=O)NCc1ccc(OC(F)(F)F)cc1F)C1CC1)CN(c1nccc(O)n1)C2]

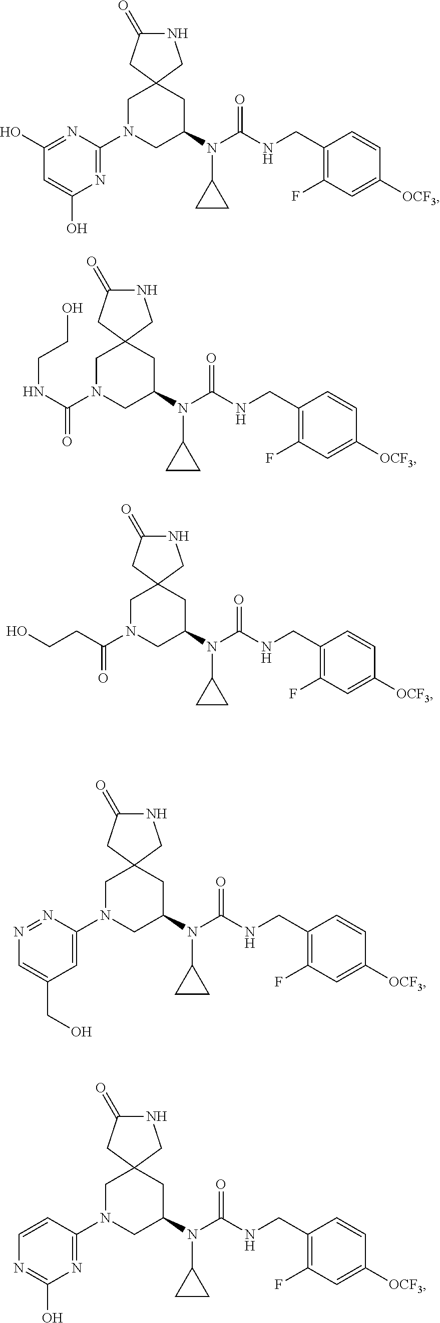

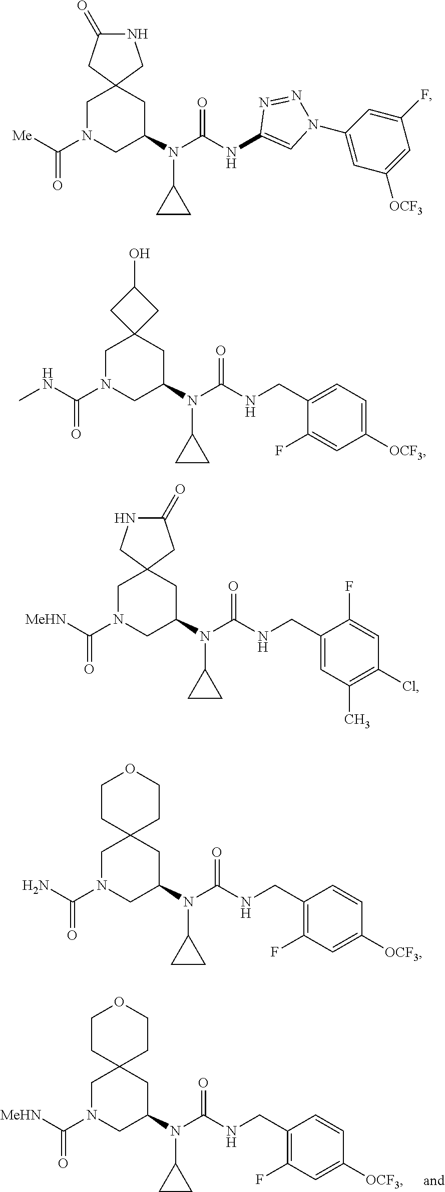

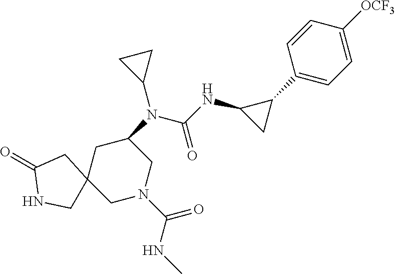 [SMILES: CNC(=O)N1C[C@H](N(C(=O)N[C@@H]2C[C@H]2c2ccc(OC(F)(F)F)cc2)C2CC2)CC2(CNC(=O)C2)C1]

(prepared from a racemic mixture of trans-cyclopropyl stereoisomers).

### Further Exemplary Compounds of Formula (I)

In certain embodiments, a compound or a pharmaceutically acceptable salt thereof having the structure of any one of the following compounds:

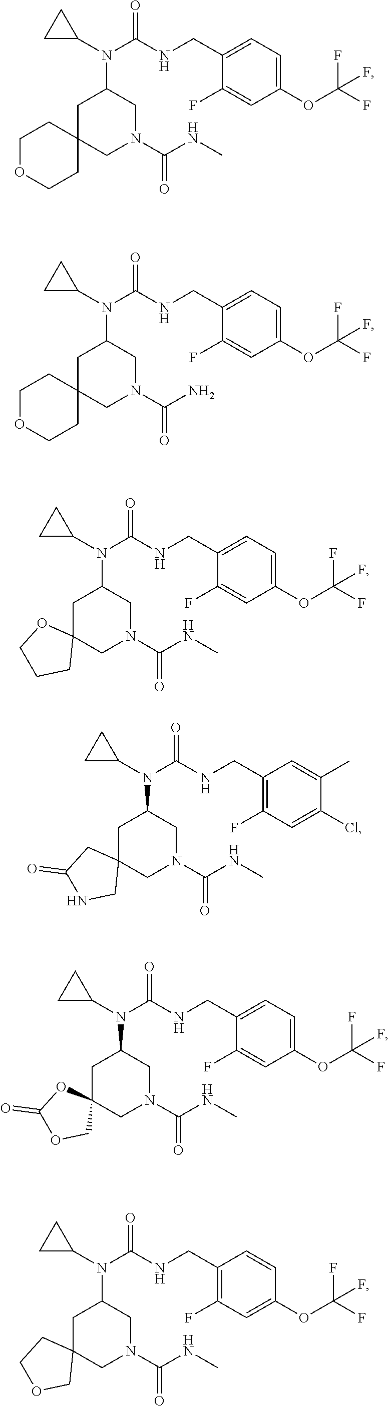

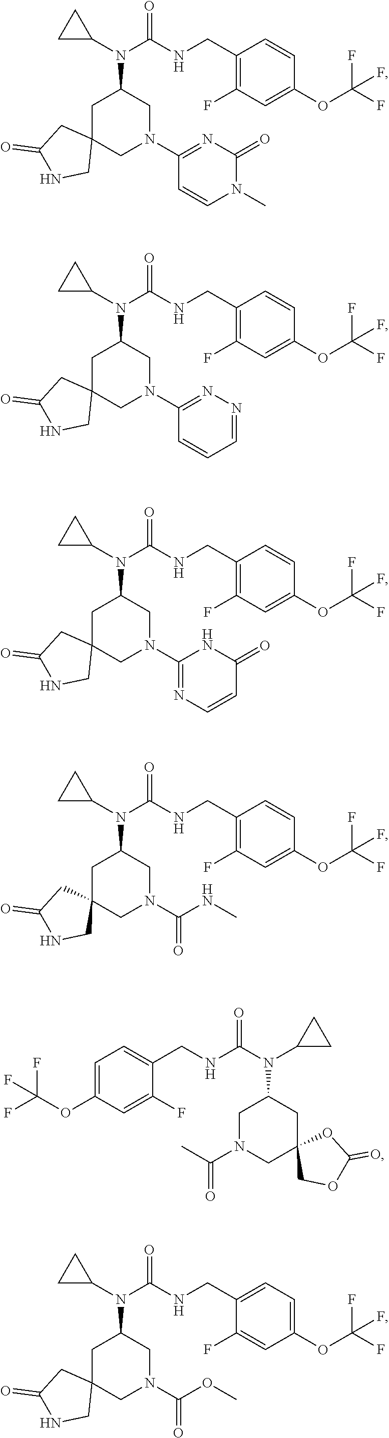 [SMILES: Cn1ccc(N2C[C@H](N(C(=O)NCc3ccc(OC(F)(F)F)cc3F)C3CC3)CC3(CNC(=O)C3)C2)nc1=O ; O=C1CC2(CN1)C[C@@H](N(C(=O)NCc1ccc(OC(F)(F)F)cc1F)C1CC1)CN(c1cccnn1)C2 ; O=C1CC2(CN1)C[C@@H](N(C(=O)NCc1ccc(OC(F)(F)F)cc1F)C1CC1)CN(c1nccc(=O)[nH]1)C2 ; CNC(=O)N1C[C@H](N(C(=O)NCc2ccc(OC(F)(F)F)cc2F)C2CC2)C[C@]2(CNC(=O)C2)C1 ; CC(=O)N1C[C@H](N(C(=O)NCc2ccc(OC(F)(F)F)cc2F)C2CC2)C[C@@]2(COC(=O)O2)C1 ; COC(=O)N1C[C@H](N(C(=O)NCc2ccc(OC(F)(F)F)cc2F)C2CC2)CC2(CNC(=O)C2)C1]

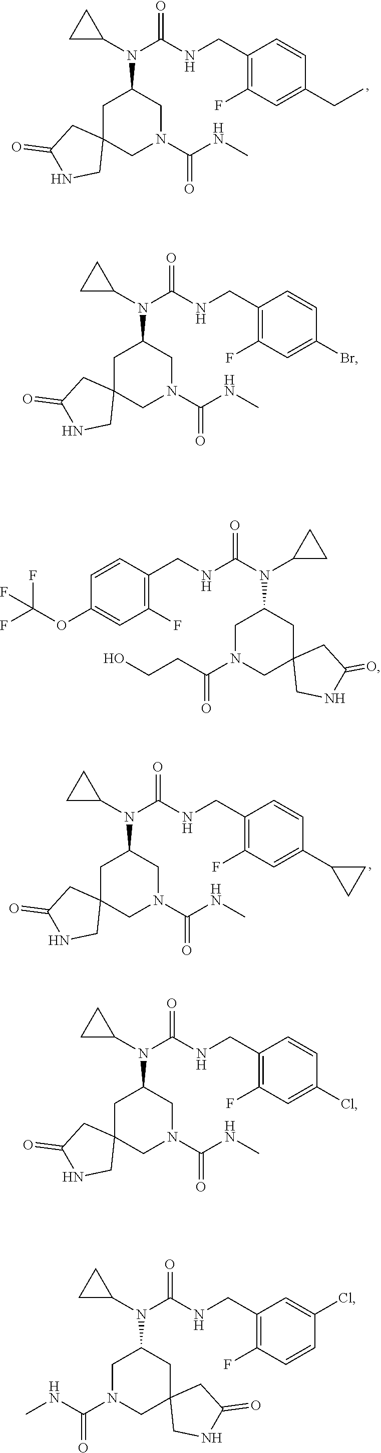

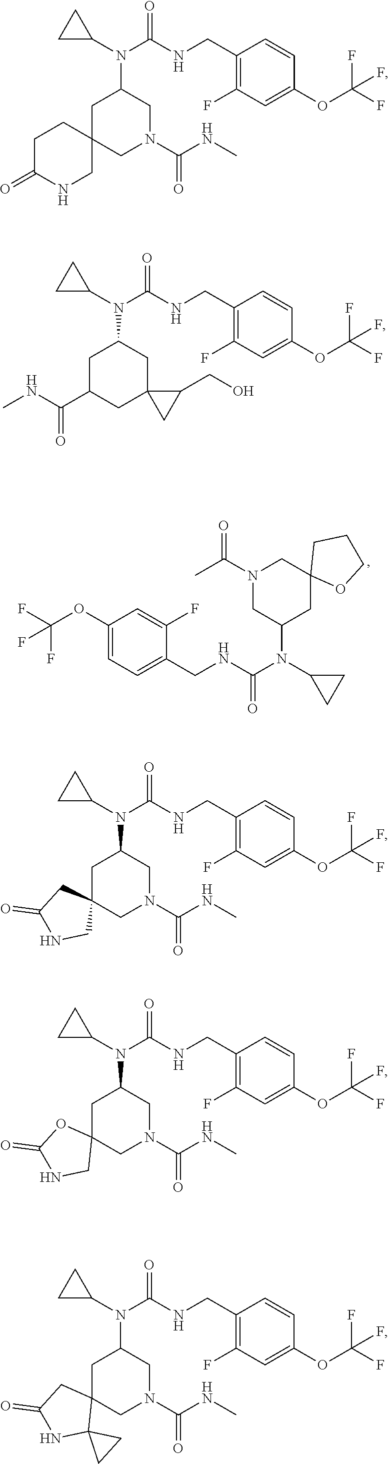 [SMILES: CNC(=O)N1CC(N(C(=O)NCc2ccc(OC(F)(F)F)cc2F)C2CC2)CC2(CCC(=O)NC2)C1 ; CNC(=O)C1C[C@H](N(C(=O)NCc2ccc(OC(F)(F)F)cc2F)C2CC2)CC2(C1)CC2CO ; CC(=O)N1CC(N(C(=O)NCc2ccc(OC(F)(F)F)cc2F)C2CC2)CC2(CCCO2)C1 ; CNC(=O)N1C[C@H](N(C(=O)NCc2ccc(OC(F)(F)F)cc2F)C2CC2)C[C@@]2(CNC(=O)C2)C1 ; CNC(=O)N1C[C@H](N(C(=O)NCc2ccc(OC(F)(F)F)cc2F)C2CC2)CC2(CNC(=O)O2)C1 ; CNC(=O)N1CC(N(C(=O)NCc2ccc(OC(F)(F)F)cc2F)C2CC2)CC2(CC(=O)NC23CC3)C1]

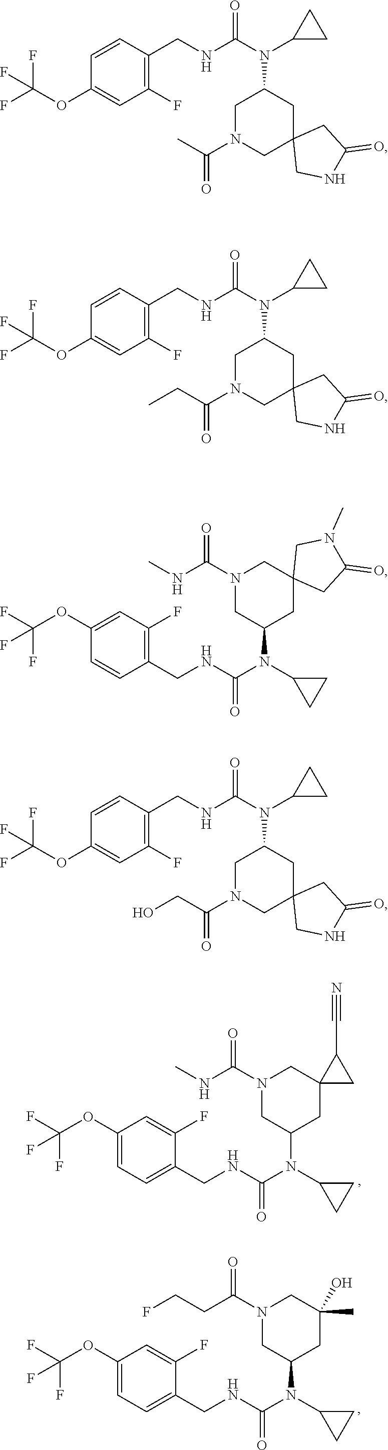 [SMILES: CC(=O)N1C[C@H](N(C(=O)NCc2ccc(OC(F)(F)F)cc2F)C2CC2)CC2(CNC(=O)C2)C1 ; CCC(=O)N1C[C@H](N(C(=O)NCc2ccc(OC(F)(F)F)cc2F)C2CC2)CC2(CNC(=O)C2)C1 ; CNC(=O)N1C[C@H](N(C(=O)NCc2ccc(OC(F)(F)F)cc2F)C2CC2)CC2(CC(=O)N(C)C2)C1 ; O=C1CC2(CN1)C[C@@H](N(C(=O)NCc1ccc(OC(F)(F)F)cc1F)C1CC1)CN(C(=O)CO)C2 ; CNC(=O)N1CC(N(C(=O)NCc2ccc(OC(F)(F)F)cc2F)C2CC2)CC2(CC2C#N)C1 ; C[C@@]1(O)C[C@@H](N(C(=O)NCc2ccc(OC(F)(F)F)cc2F)C2CC2)CN(C(=O)CCF)C1]

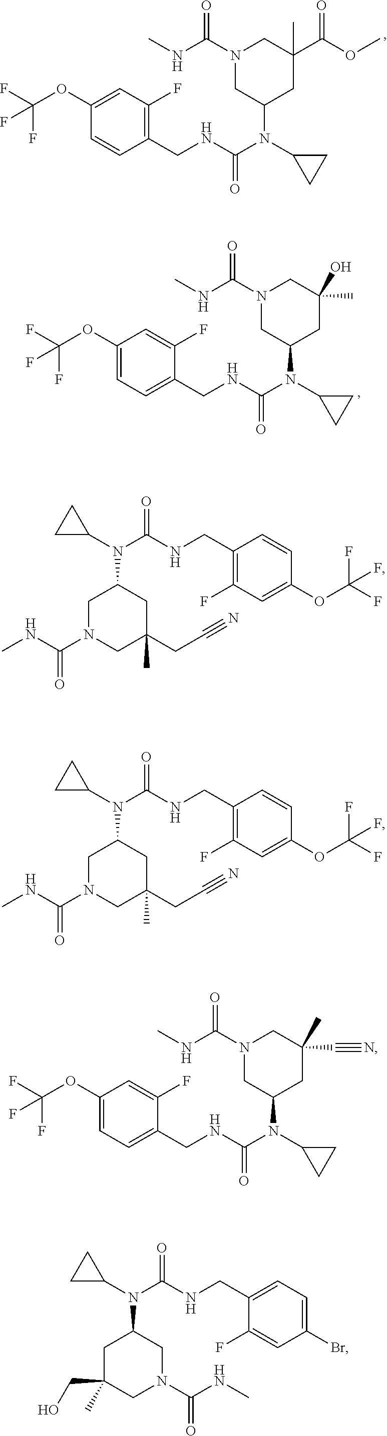

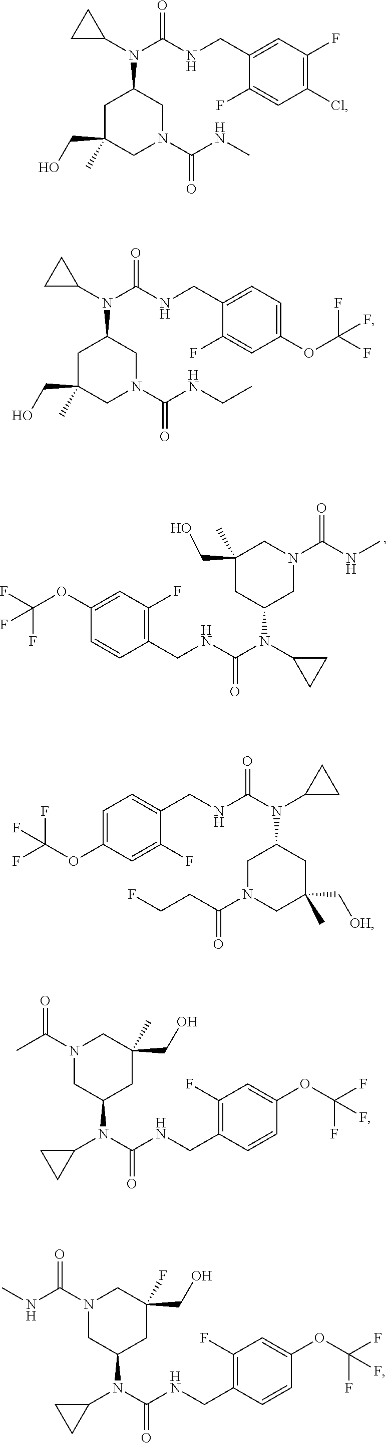

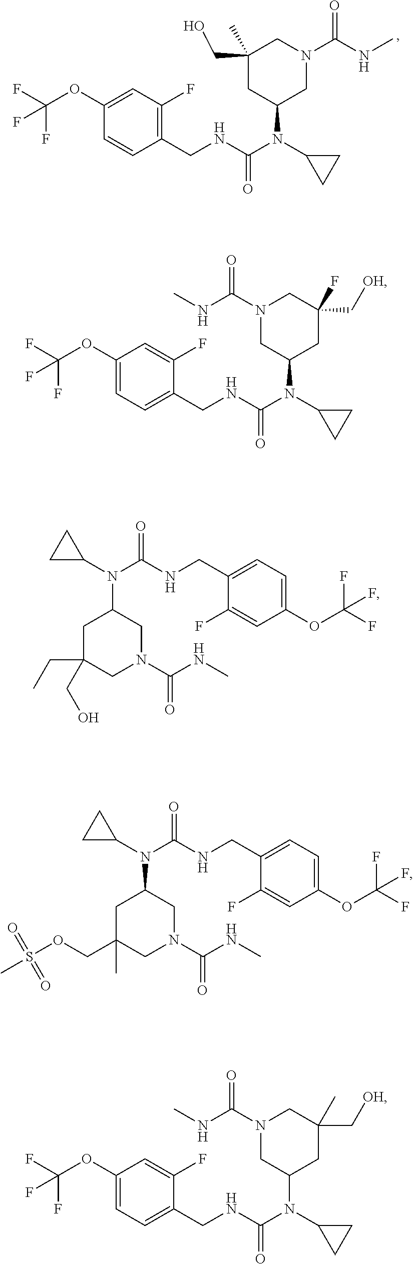

### Exemplary Compounds of Formula (II)

In certain embodiments, a compound or a pharmaceutically acceptable salt thereof having the structure of any one of the following compounds:

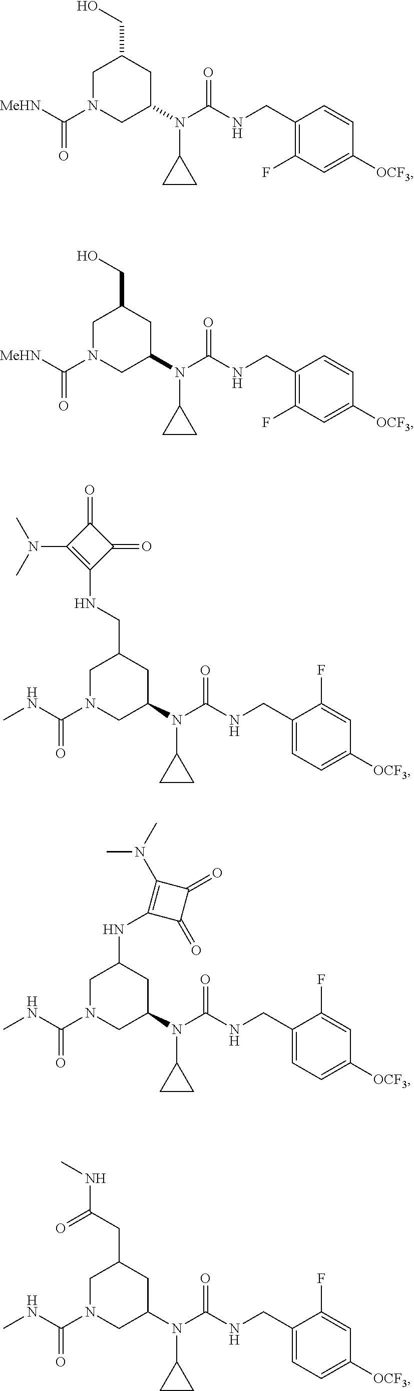

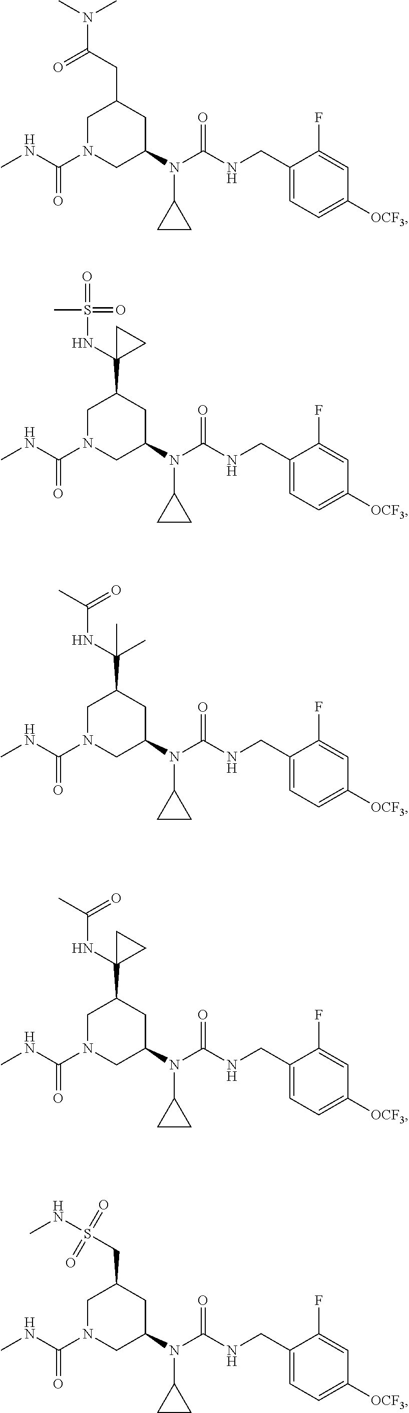 [SMILES: CNC(=O)N1CC(CC(=O)N(C)C)C[C@@H](N(C(=O)NCc2ccc(OC(F)(F)F)cc2F)C2CC2)C1 ; CNC(=O)N1C[C@H](N(C(=O)NCc2ccc(OC(F)(F)F)cc2F)C2CC2)C[C@H](C2(NS(C)(=O)=O)CC2)C1 ; CNC(=O)N1C[C@H](N(C(=O)NCc2ccc(OC(F)(F)F)cc2F)C2CC2)C[C@H](C(C)(C)NC(C)=O)C1 ; CNC(=O)N1C[C@H](N(C(=O)NCc2ccc(OC(F)(F)F)cc2F)C2CC2)C[C@H](C2(NC(C)=O)CC2)C1 ; CNC(=O)N1C[C@@H](CS(=O)(=O)NC)C[C@@H](N(C(=O)NCc2ccc(OC(F)(F)F)cc2F)C2CC2)C1]

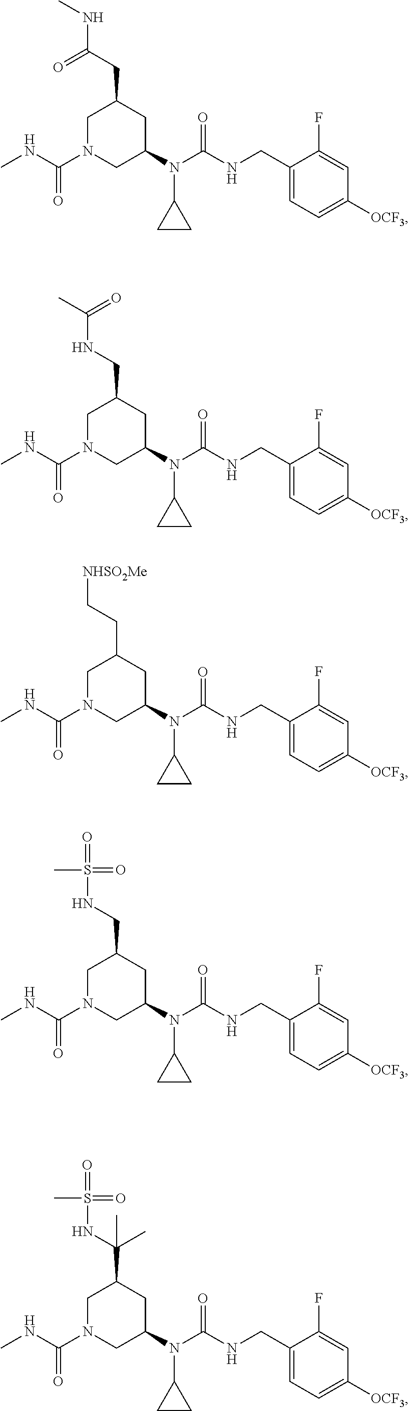

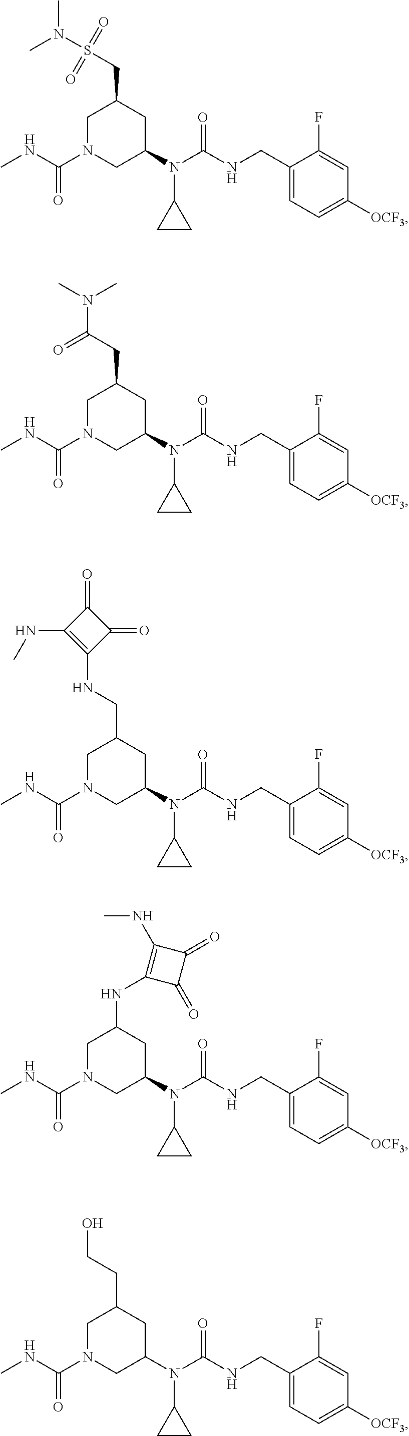 [SMILES: CNC(=O)N1C[C@@H](CS(=O)(=O)N(C)C)C[C@@H](N(C(=O)NCc2ccc(OC(F)(F)F)cc2F)C2CC2)C1 ; CNC(=O)N1C[C@@H](CC(=O)N(C)C)C[C@@H](N(C(=O)NCc2ccc(OC(F)(F)F)cc2F)C2CC2)C1 ; CNC(=O)N1CC(CNc2c(NC)c(=O)c2=O)C[C@@H](N(C(=O)NCc2ccc(OC(F)(F)F)cc2F)C2CC2)C1 ; CNC(=O)N1CC(Nc2c(NC)c(=O)c2=O)C[C@@H](N(C(=O)NCc2ccc(OC(F)(F)F)cc2F)C2CC2)C1 ; CNC(=O)N1CC(CCO)CC(N(C(=O)NCc2ccc(OC(F)(F)F)cc2F)C2CC2)C1]

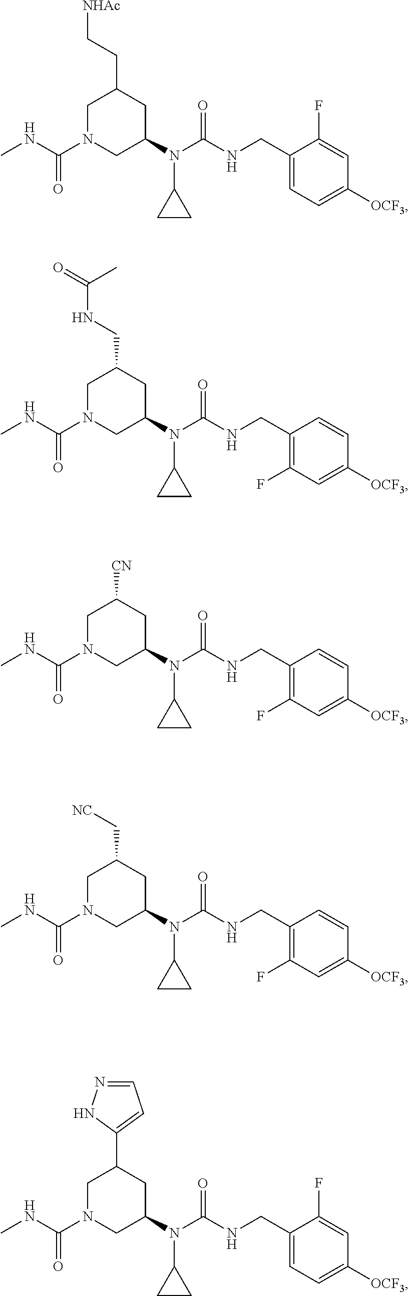 [SMILES: CNC(=O)N1CC(CCNC(C)=O)C[C@@H](N(C(=O)NCc2ccc(OC(F)(F)F)cc2F)C2CC2)C1 ; CNC(=O)N1C[C@H](CNC(C)=O)C[C@@H](N(C(=O)NCc2ccc(OC(F)(F)F)cc2F)C2CC2)C1 ; CNC(=O)N1C[C@H](C#N)C[C@@H](N(C(=O)NCc2ccc(OC(F)(F)F)cc2F)C2CC2)C1 ; CNC(=O)N1C[C@H](CC#N)C[C@@H](N(C(=O)NCc2ccc(OC(F)(F)F)cc2F)C2CC2)C1 ; CNC(=O)N1CC(c2ccn[nH]2)C[C@@H](N(C(=O)NCc2ccc(OC(F)(F)F)cc2F)C2CC2)C1]

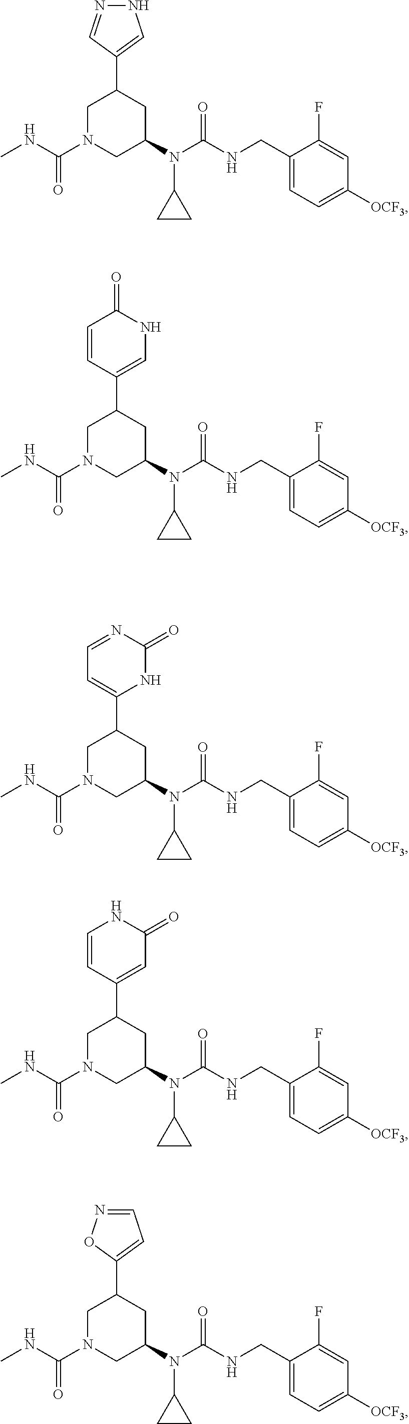 [SMILES: CNC(=O)N1CC(c2cn[nH]c2)C[C@@H](N(C(=O)NCc2ccc(OC(F)(F)F)cc2F)C2CC2)C1 ; CNC(=O)N1CC(c2ccc(=O)[nH]c2)C[C@@H](N(C(=O)NCc2ccc(OC(F)(F)F)cc2F)C2CC2)C1 ; CNC(=O)N1CC(c2ccnc(=O)[nH]2)C[C@@H](N(C(=O)NCc2ccc(OC(F)(F)F)cc2F)C2CC2)C1 ; CNC(=O)N1CC(c2cc[nH]c(=O)c2)C[C@@H](N(C(=O)NCc2ccc(OC(F)(F)F)cc2F)C2CC2)C1 ; CNC(=O)N1CC(c2ccno2)C[C@@H](N(C(=O)NCc2ccc(OC(F)(F)F)cc2F)C2CC2)C1]

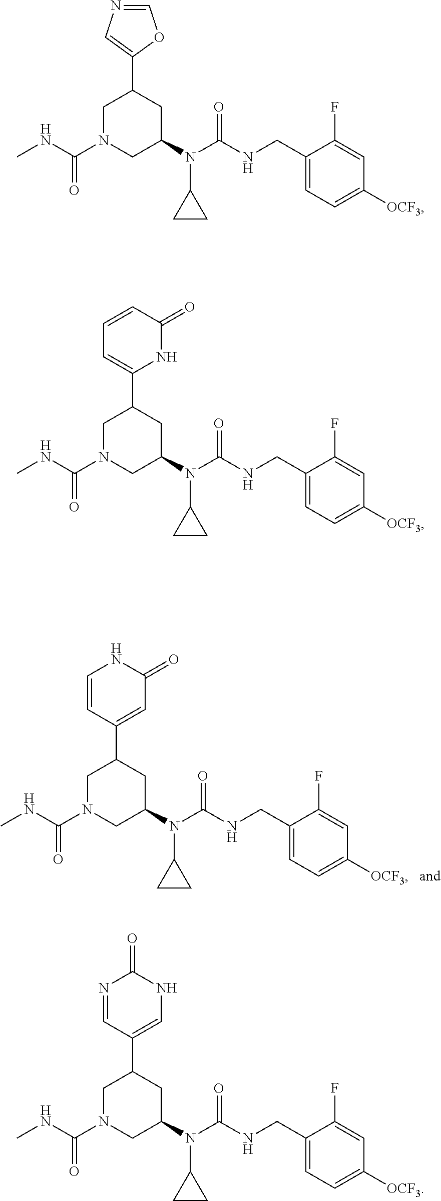 [SMILES: CNC(=O)N1CC(c2cnco2)C[C@@H](N(C(=O)NCc2ccc(OC(F)(F)F)cc2F)C2CC2)C1 ; CNC(=O)N1CC(c2cccc(=O)[nH]2)C[C@@H](N(C(=O)NCc2ccc(OC(F)(F)F)cc2F)C2CC2)C1 ; CNC(=O)N1CC(c2cc[nH]c(=O)c2)C[C@@H](N(C(=O)NCc2ccc(OC(F)(F)F)cc2F)C2CC2)C1 ; CNC(=O)N1CC(c2cnc(=O)[nH]c2)C[C@@H](N(C(=O)NCc2ccc(OC(F)(F)F)cc2F)C2CC2)C1]

### Further Exemplary Compounds of Formula (II)

In certain embodiments, a compound or a pharmaceutically acceptable salt thereof having the structure of any one of the following compounds:

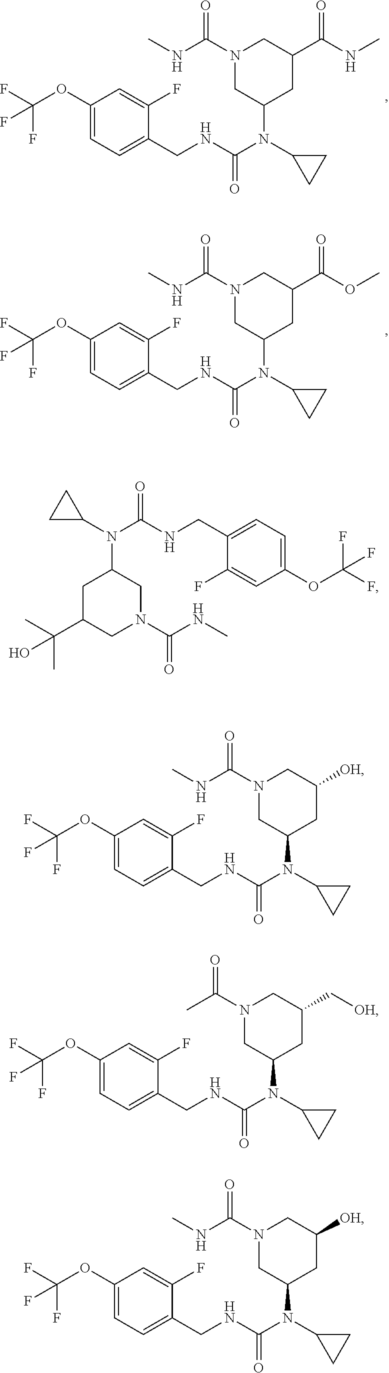

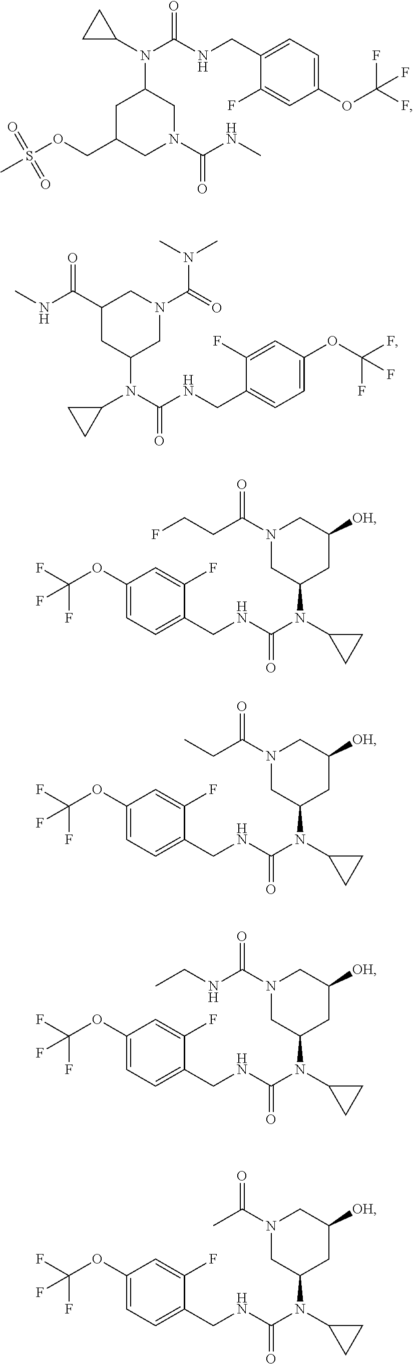 [SMILES: CNC(=O)N1CC(COS(C)(=O)=O)CC(N(C(=O)NCc2ccc(OC(F)(F)F)cc2F)C2CC2)C1 ; CNC(=O)C1CC(N(C(=O)NCc2ccc(OC(F)(F)F)cc2F)C2CC2)CN(C(=O)N(C)C)C1 ; O=C(CCF)N1C[C@@H](O)C[C@@H](N(C(=O)NCc2ccc(OC(F)(F)F)cc2F)C2CC2)C1 ; CCC(=O)N1C[C@@H](O)C[C@@H](N(C(=O)NCc2ccc(OC(F)(F)F)cc2F)C2CC2)C1 ; CCNC(=O)N1C[C@@H](O)C[C@@H](N(C(=O)NCc2ccc(OC(F)(F)F)cc2F)C2CC2)C1 ; CC(=O)N1C[C@@H](O)C[C@@H](N(C(=O)NCc2ccc(OC(F)(F)F)cc2F)C2CC2)C1]

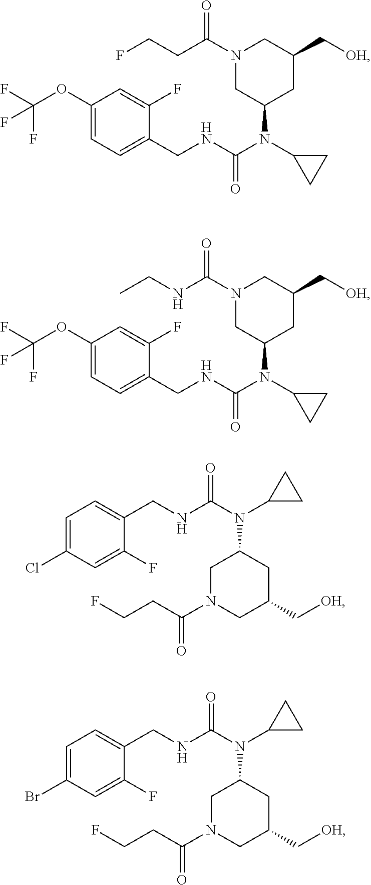 [SMILES: O=C(CCF)N1C[C@@H](CO)C[C@@H](N(C(=O)NCc2ccc(OC(F)(F)F)cc2F)C2CC2)C1 ; CCNC(=O)N1C[C@@H](CO)C[C@@H](N(C(=O)NCc2ccc(OC(F)(F)F)cc2F)C2CC2)C1 ; O=C(CCF)N1C[C@@H](CO)C[C@@H](N(C(=O)NCc2ccc(Cl)cc2F)C2CC2)C1 ; O=C(CCF)N1C[C@@H](CO)C[C@@H](N(C(=O)NCc2ccc(Br)cc2F)C2CC2)C1]

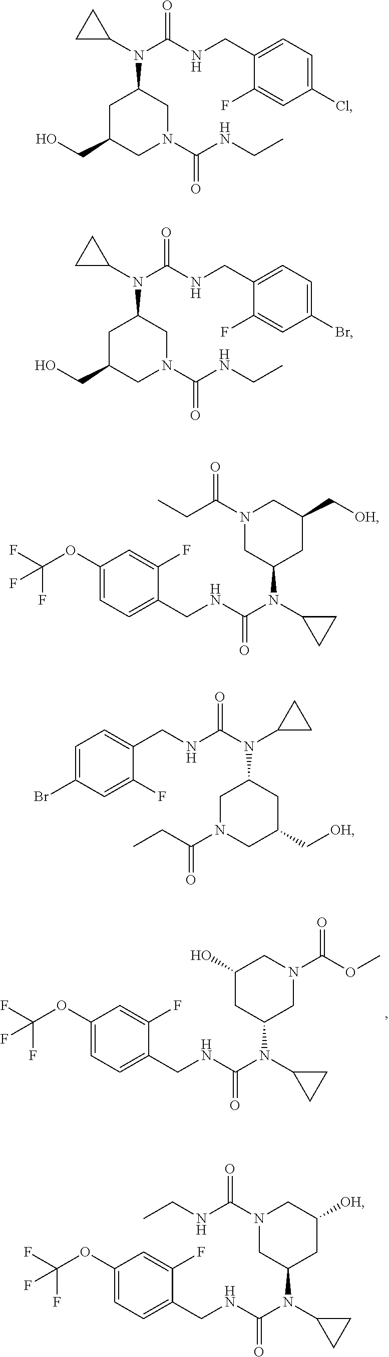 [SMILES: CCNC(=O)N1C[C@@H](CO)C[C@@H](N(C(=O)NCc2ccc(Cl)cc2F)C2CC2)C1 ; CCNC(=O)N1C[C@@H](CO)C[C@@H](N(C(=O)NCc2ccc(Br)cc2F)C2CC2)C1 ; CCC(=O)N1C[C@@H](CO)C[C@@H](N(C(=O)NCc2ccc(OC(F)(F)F)cc2F)C2CC2)C1 ; CCC(=O)N1C[C@@H](CO)C[C@@H](N(C(=O)NCc2ccc(Br)cc2F)C2CC2)C1 ; COC(=O)N1C[C@@H](O)C[C@@H](N(C(=O)NCc2ccc(OC(F)(F)F)cc2F)C2CC2)C1 ; CCNC(=O)N1C[C@H](O)C[C@@H](N(C(=O)NCc2ccc(OC(F)(F)F)cc2F)C2CC2)C1]

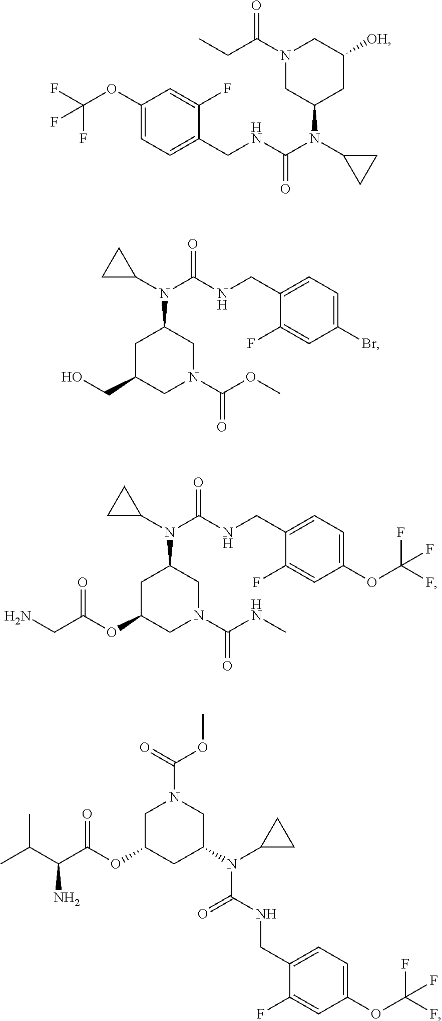 [SMILES: CCC(=O)N1C[C@H](O)C[C@@H](N(C(=O)NCc2ccc(OC(F)(F)F)cc2F)C2CC2)C1 ; COC(=O)N1C[C@@H](CO)C[C@@H](N(C(=O)NCc2ccc(Br)cc2F)C2CC2)C1 ; CNC(=O)N1C[C@@H](OC(=O)CN)C[C@@H](N(C(=O)NCc2ccc(OC(F)(F)F)cc2F)C2CC2)C1 ; COC(=O)N1C[C@@H](OC(=O)[C@@H](N)C(C)C)C[C@@H](N(C(=O)NCc2ccc(OC(F)(F)F)cc2F)C2CC2)C1]

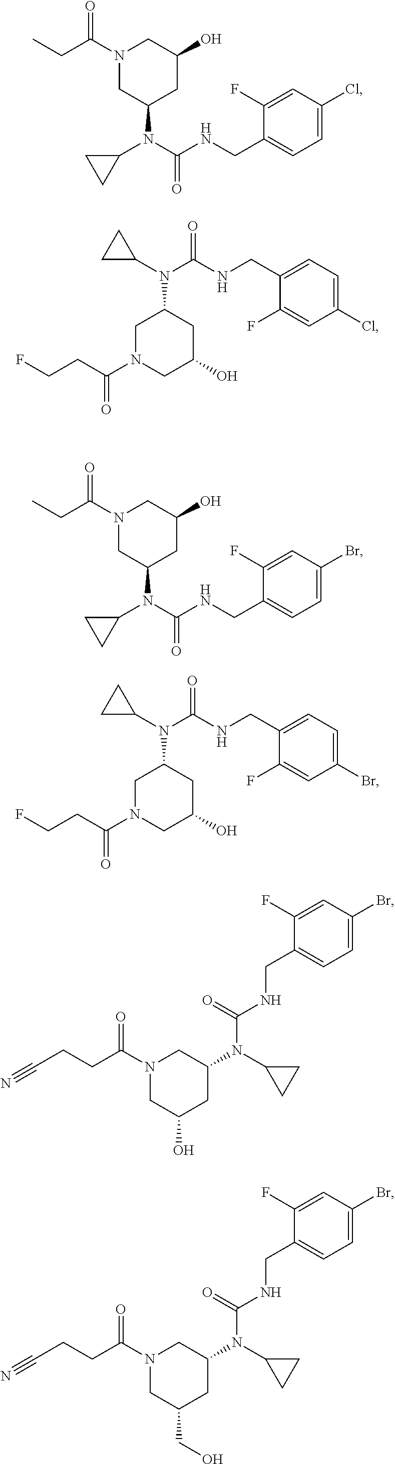

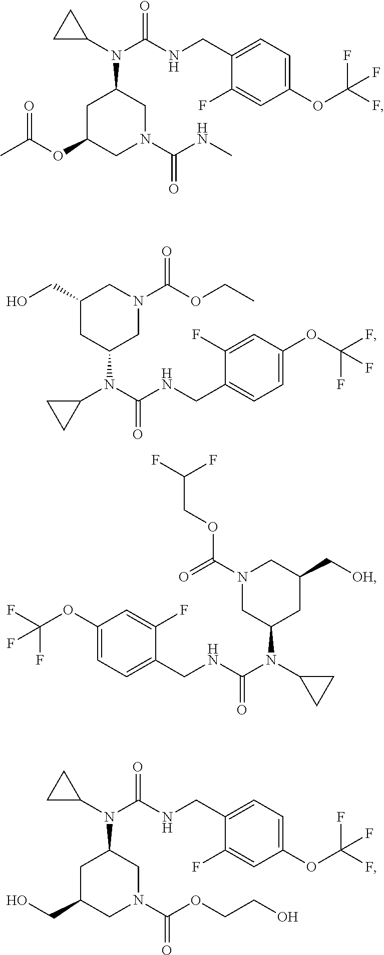

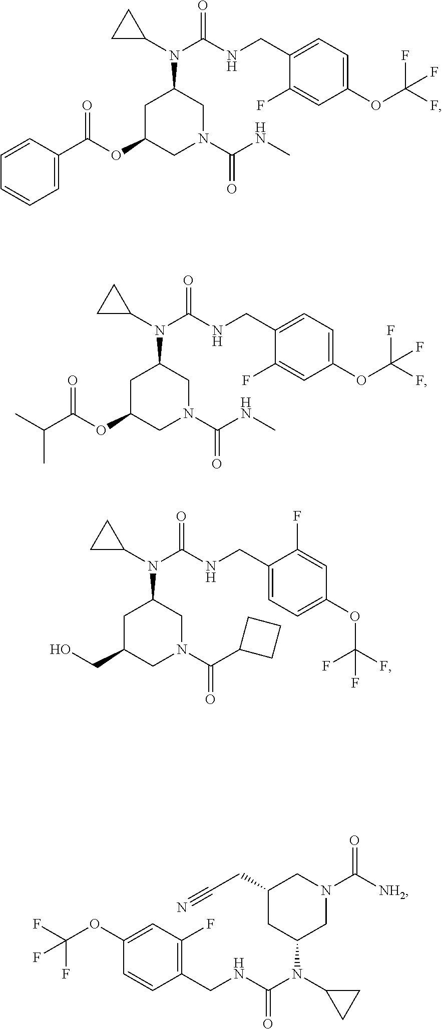 [SMILES: CNC(=O)N1C[C@@H](OC(=O)c2ccccc2)C[C@@H](N(C(=O)NCc2ccc(OC(F)(F)F)cc2F)C2CC2)C1 ; CNC(=O)N1C[C@@H](OC(=O)C(C)C)C[C@@H](N(C(=O)NCc2ccc(OC(F)(F)F)cc2F)C2CC2)C1 ; O=C(C1CCC1)N1C[C@@H](CO)C[C@@H](N(C(=O)NCc2ccc(OC(F)(F)F)cc2F)C2CC2)C1 ; N#CC[C@H]1C[C@@H](N(C(=O)NCc2ccc(OC(F)(F)F)cc2F)C2CC2)CN(C(N)=O)C1]

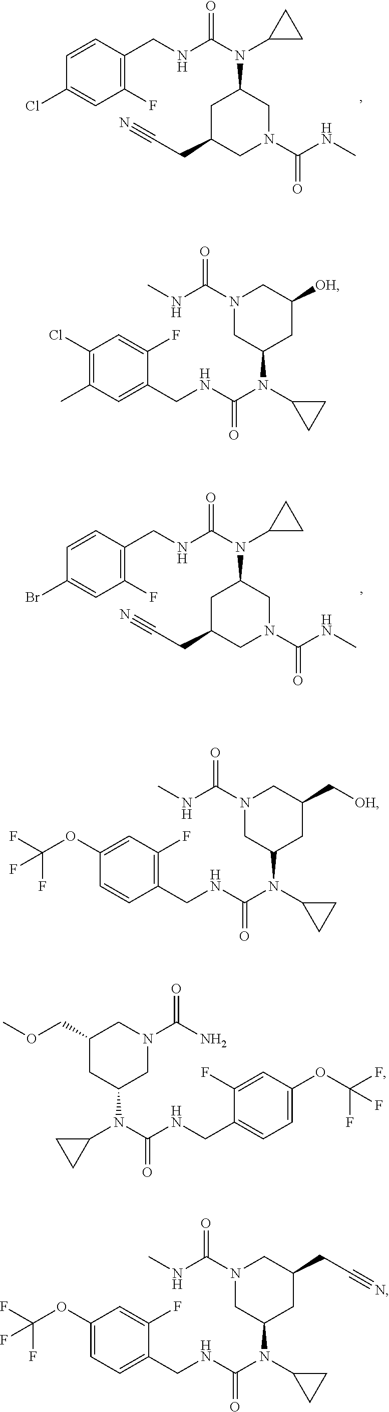

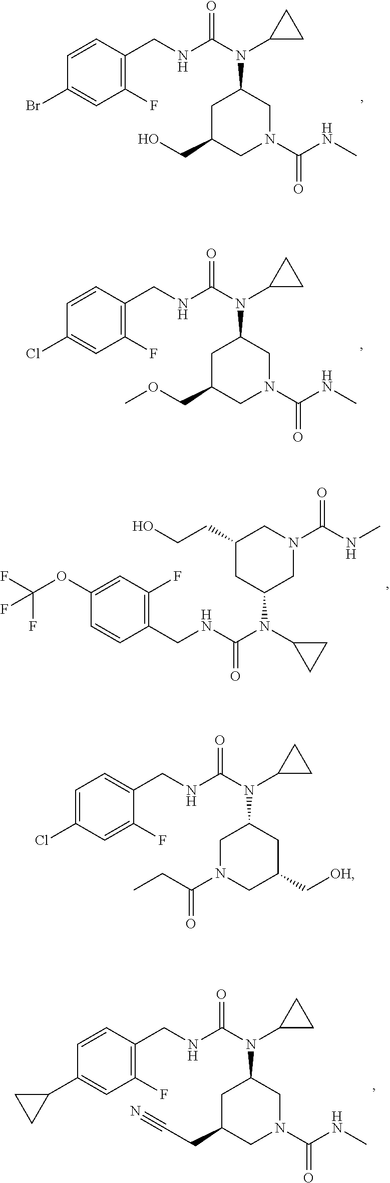 [SMILES: CNC(=O)N1C[C@@H](CO)C[C@@H](N(C(=O)NCc2ccc(Br)cc2F)C2CC2)C1 ; CNC(=O)N1C[C@@H](COC)C[C@@H](N(C(=O)NCc2ccc(Cl)cc2F)C2CC2)C1 ; CNC(=O)N1C[C@@H](CCO)C[C@@H](N(C(=O)NCc2ccc(OC(F)(F)F)cc2F)C2CC2)C1 ; CCC(=O)N1C[C@@H](CO)C[C@@H](N(C(=O)NCc2ccc(Cl)cc2F)C2CC2)C1 ; CNC(=O)N1C[C@@H](CC#N)C[C@@H](N(C(=O)NCc2ccc(C3CC3)cc2F)C2CC2)C1]

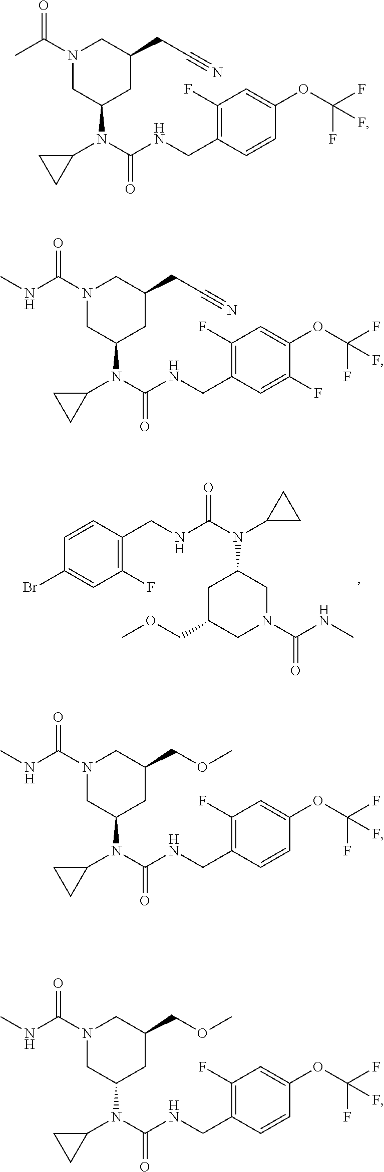

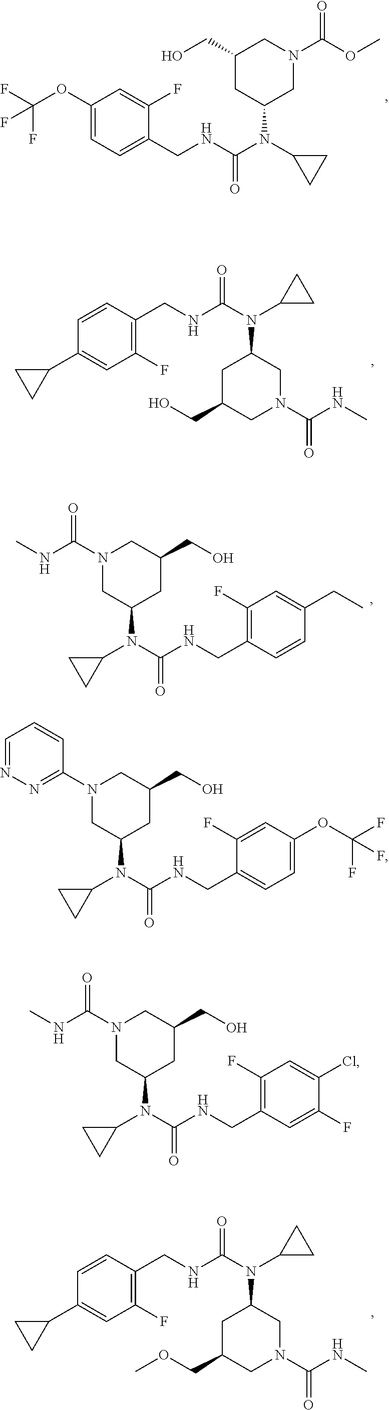

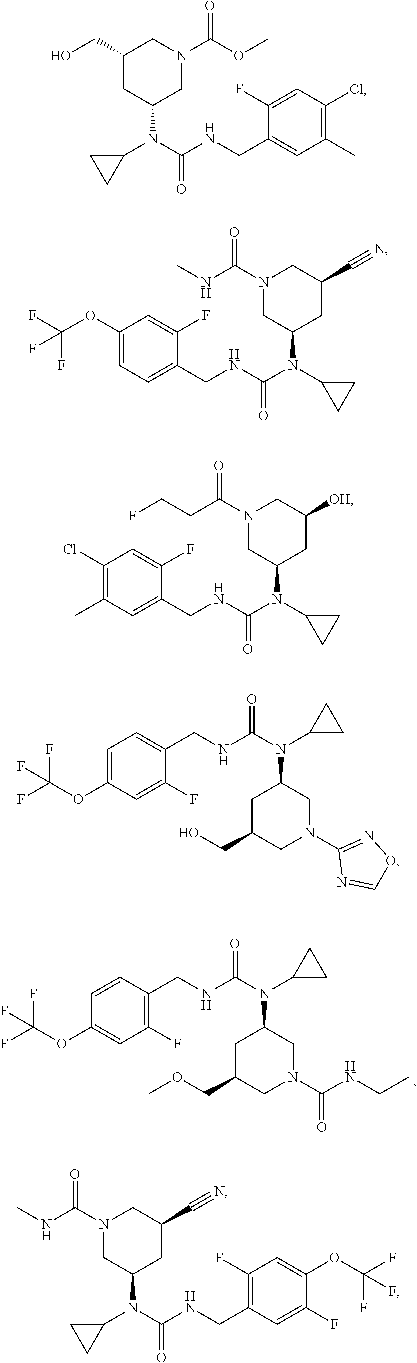

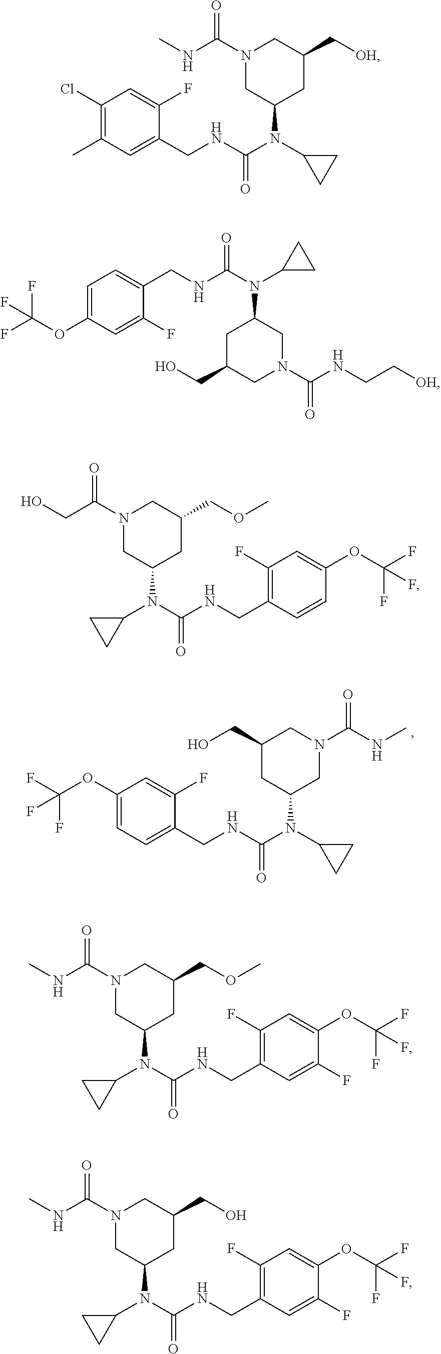

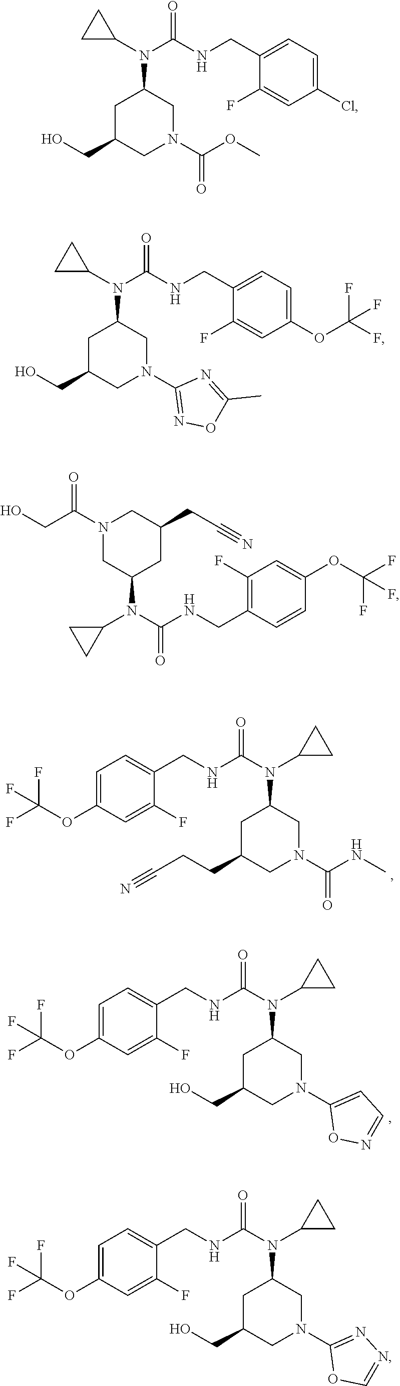 [SMILES: COC(=O)N1C[C@@H](CO)C[C@@H](N(C(=O)NCc2ccc(Cl)cc2F)C2CC2)C1 ; Cc1nc(N2C[C@@H](CO)C[C@@H](N(C(=O)NCc3ccc(OC(F)(F)F)cc3F)C3CC3)C2)no1 ; N#CC[C@H]1C[C@@H](N(C(=O)NCc2ccc(OC(F)(F)F)cc2F)C2CC2)CN(C(=O)CO)C1 ; CNC(=O)N1C[C@@H](CCC#N)C[C@@H](N(C(=O)NCc2ccc(OC(F)(F)F)cc2F)C2CC2)C1 ; O=C(NCc1ccc(OC(F)(F)F)cc1F)N(C1CC1)[C@@H]1C[C@H](CO)CN(c2ccno2)C1 ; O=C(NCc1ccc(OC(F)(F)F)cc1F)N(C1CC1)[C@@H]1C[C@H](CO)CN(c2nnco2)C1]

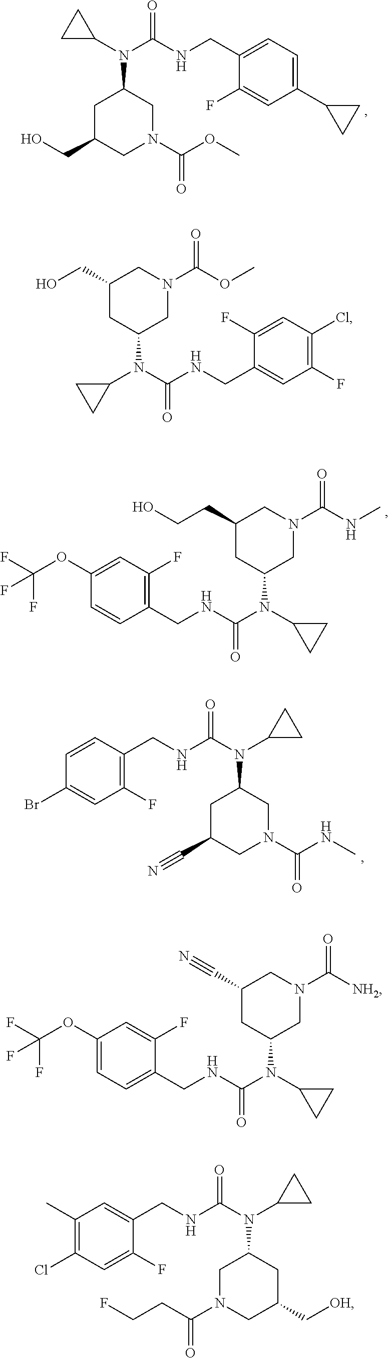 [SMILES: COC(=O)N1C[C@@H](CO)C[C@@H](N(C(=O)NCc2ccc(C3CC3)cc2F)C2CC2)C1 ; COC(=O)N1C[C@@H](CO)C[C@@H](N(C(=O)NCc2cc(F)c(Cl)cc2F)C2CC2)C1 ; CNC(=O)N1C[C@H](CCO)C[C@@H](N(C(=O)NCc2ccc(OC(F)(F)F)cc2F)C2CC2)C1 ; CNC(=O)N1C[C@@H](C#N)C[C@@H](N(C(=O)NCc2ccc(Br)cc2F)C2CC2)C1 ; N#C[C@H]1C[C@@H](N(C(=O)NCc2ccc(OC(F)(F)F)cc2F)C2CC2)CN(C(N)=O)C1 ; Cc1cc(CNC(=O)N(C2CC2)[C@@H]2C[C@H](CO)CN(C(=O)CCF)C2)c(F)cc1Cl]

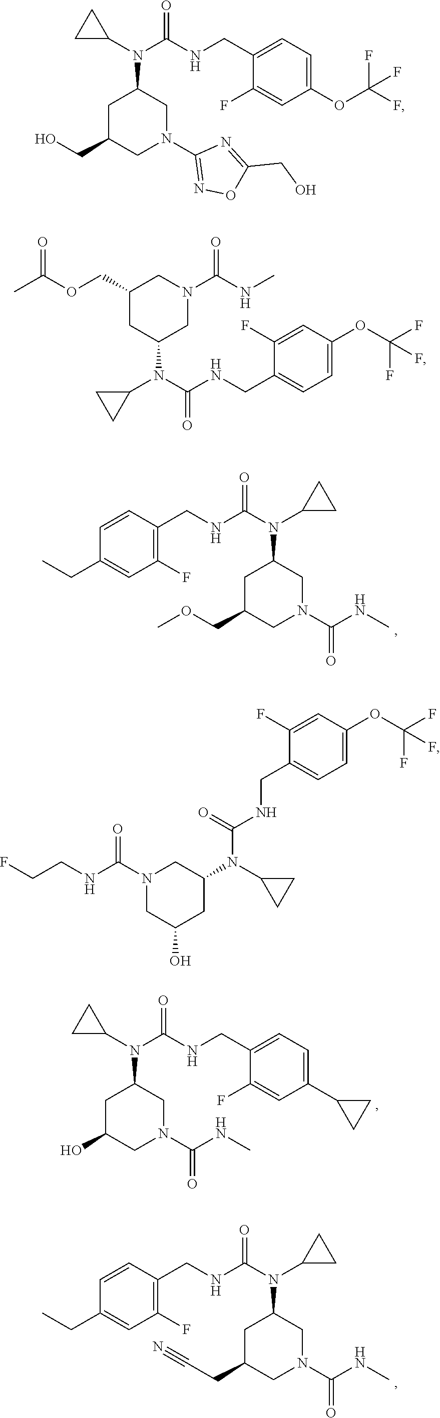

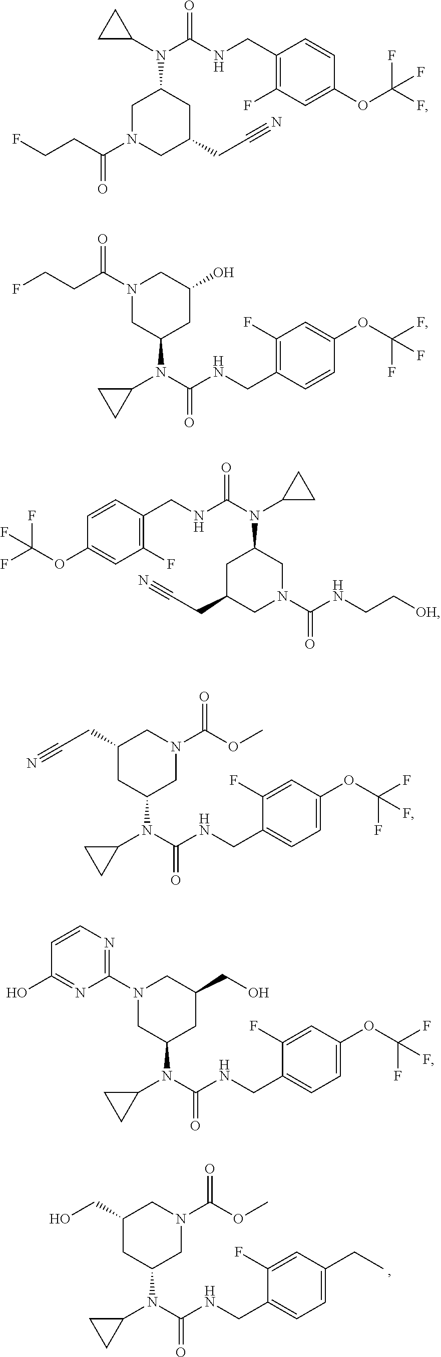

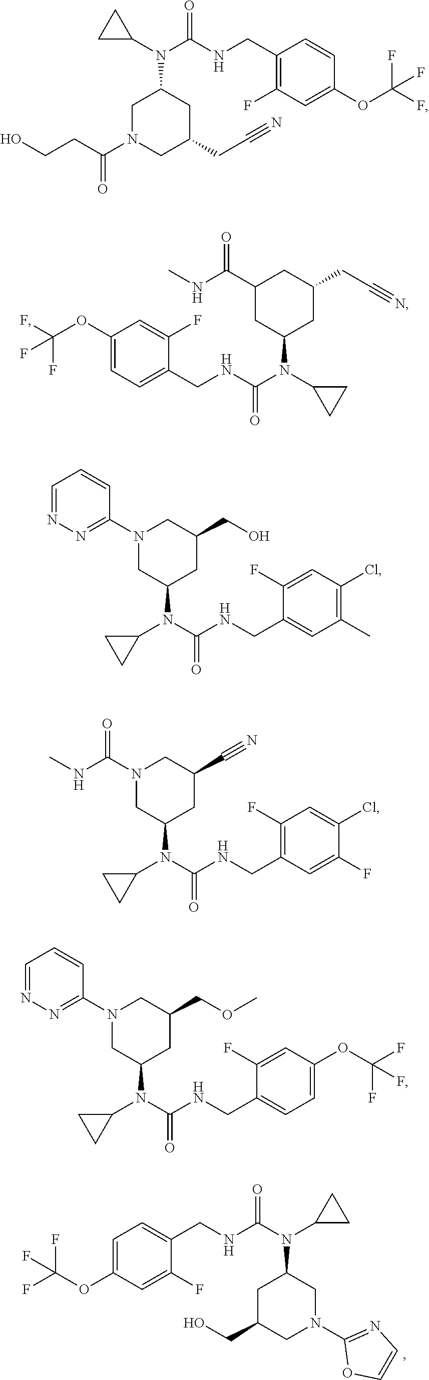

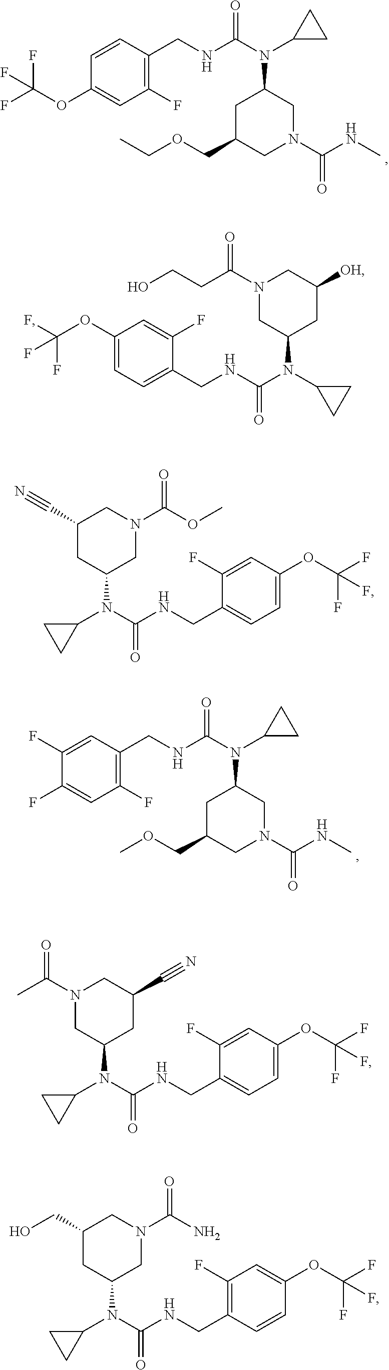

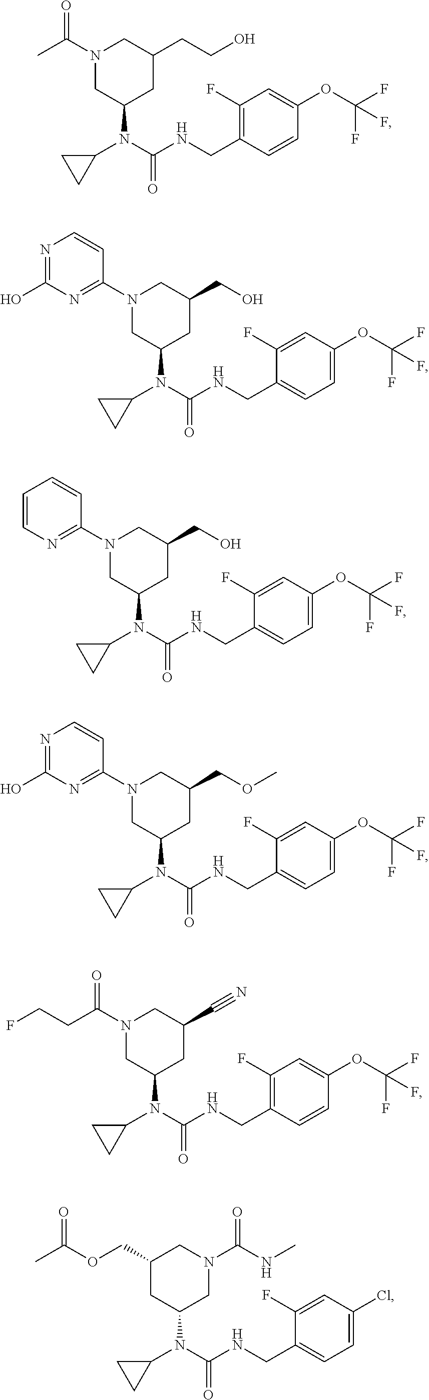 [SMILES: CC(=O)N1CC(CCO)C[C@@H](N(C(=O)NCc2ccc(OC(F)(F)F)cc2F)C2CC2)C1 ; O=C(NCc1ccc(OC(F)(F)F)cc1F)N(C1CC1)[C@@H]1C[C@H](CO)CN(c2ccnc(O)n2)C1 ; O=C(NCc1ccc(OC(F)(F)F)cc1F)N(C1CC1)[C@@H]1C[C@H](CO)CN(c2ccccn2)C1 ; COC[C@H]1C[C@@H](N(C(=O)NCc2ccc(OC(F)(F)F)cc2F)C2CC2)CN(c2ccnc(O)n2)C1 ; N#C[C@H]1C[C@@H](N(C(=O)NCc2ccc(OC(F)(F)F)cc2F)C2CC2)CN(C(=O)CCF)C1 ; CNC(=O)N1C[C@@H](COC(C)=O)C[C@@H](N(C(=O)NCc2ccc(Cl)cc2F)C2CC2)C1]

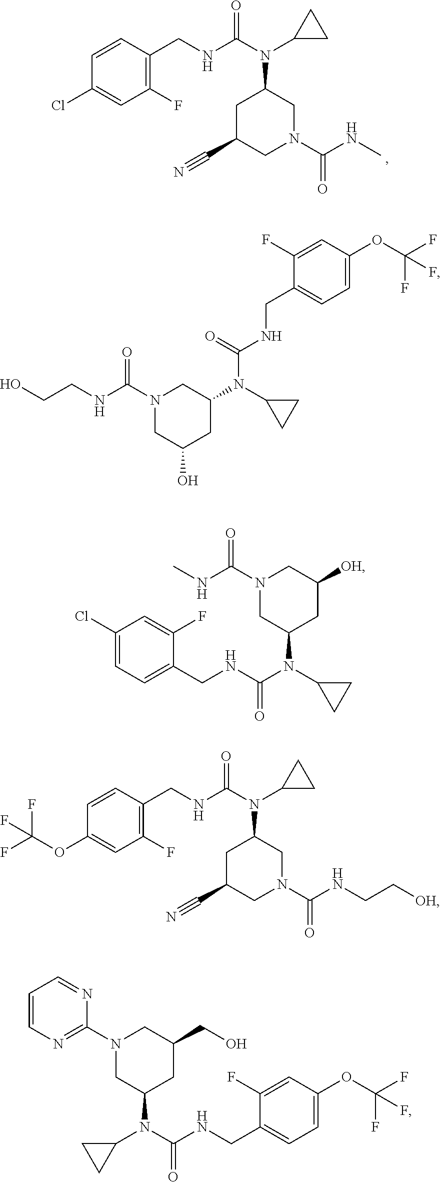 [SMILES: CNC(=O)N1C[C@@H](C#N)C[C@@H](N(C(=O)NCc2ccc(Cl)cc2F)C2CC2)C1 ; O=C(NCCO)N1C[C@@H](O)C[C@@H](N(C(=O)NCc2ccc(OC(F)(F)F)cc2F)C2CC2)C1 ; CNC(=O)N1C[C@@H](O)C[C@@H](N(C(=O)NCc2ccc(Cl)cc2F)C2CC2)C1 ; N#C[C@H]1C[C@@H](N(C(=O)NCc2ccc(OC(F)(F)F)cc2F)C2CC2)CN(C(=O)NCCO)C1 ; O=C(NCc1ccc(OC(F)(F)F)cc1F)N(C1CC1)[C@@H]1C[C@H](CO)CN(c2ncccn2)C1]

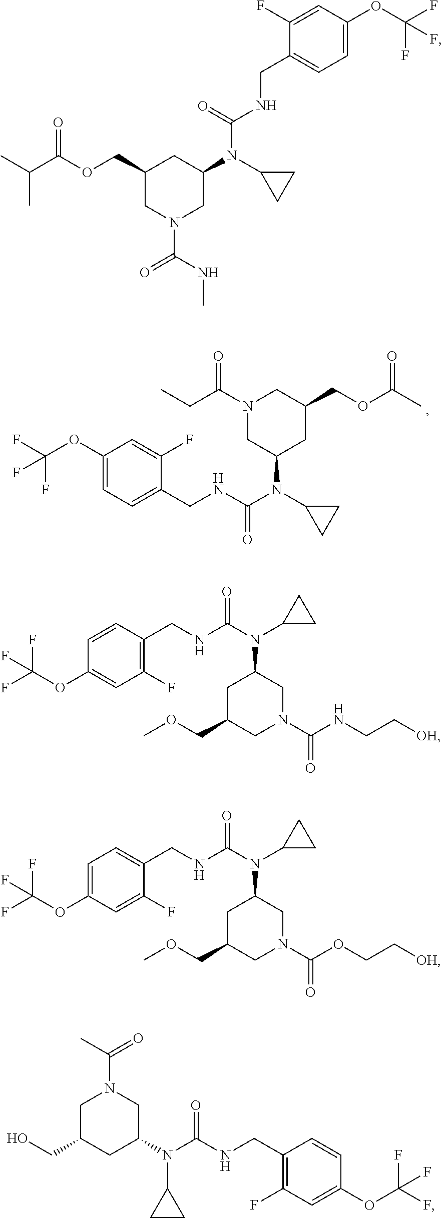 [SMILES: CNC(=O)N1C[C@@H](COC(=O)C(C)C)C[C@@H](N(C(=O)NCc2ccc(OC(F)(F)F)cc2F)C2CC2)C1 ; CCC(=O)N1C[C@@H](COC(C)=O)C[C@@H](N(C(=O)NCc2ccc(OC(F)(F)F)cc2F)C2CC2)C1 ; COC[C@H]1C[C@@H](N(C(=O)NCc2ccc(OC(F)(F)F)cc2F)C2CC2)CN(C(=O)NCCO)C1 ; COC[C@H]1C[C@@H](N(C(=O)NCc2ccc(OC(F)(F)F)cc2F)C2CC2)CN(C(=O)OCCO)C1 ; CC(=O)N1C[C@@H](CO)C[C@@H](N(C(=O)NCc2ccc(OC(F)(F)F)cc2F)C2CC2)C1]

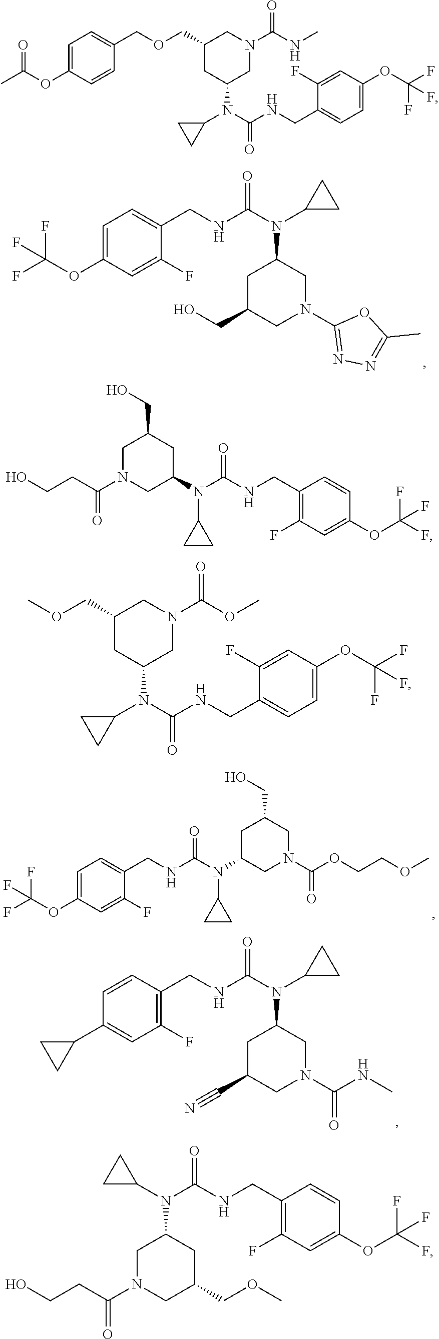

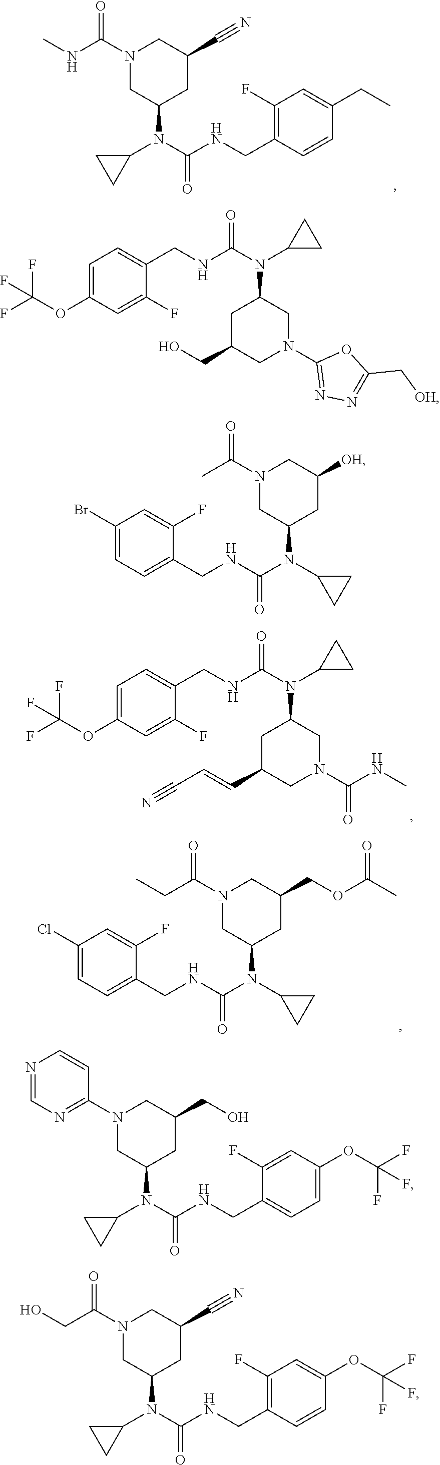

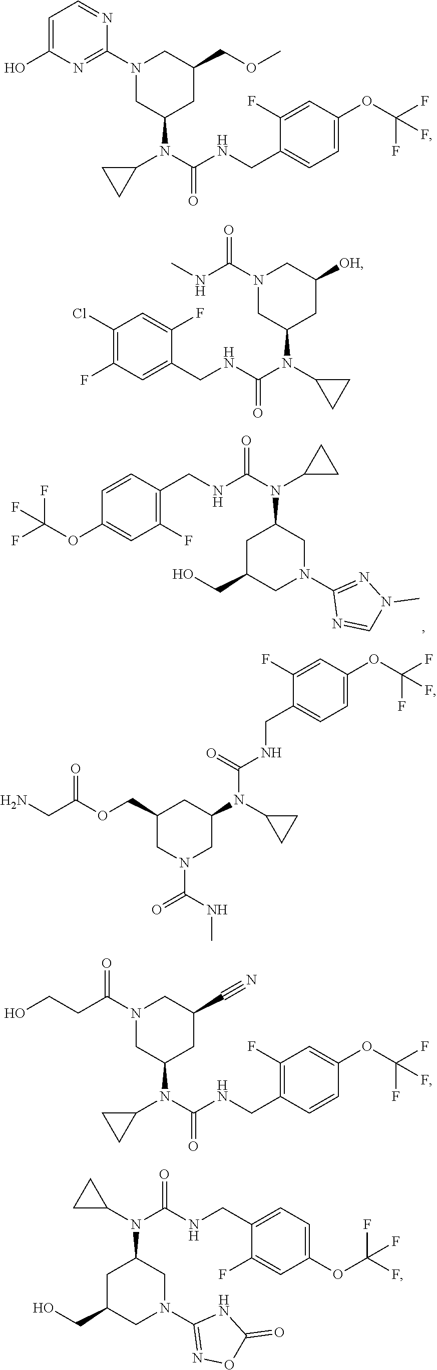

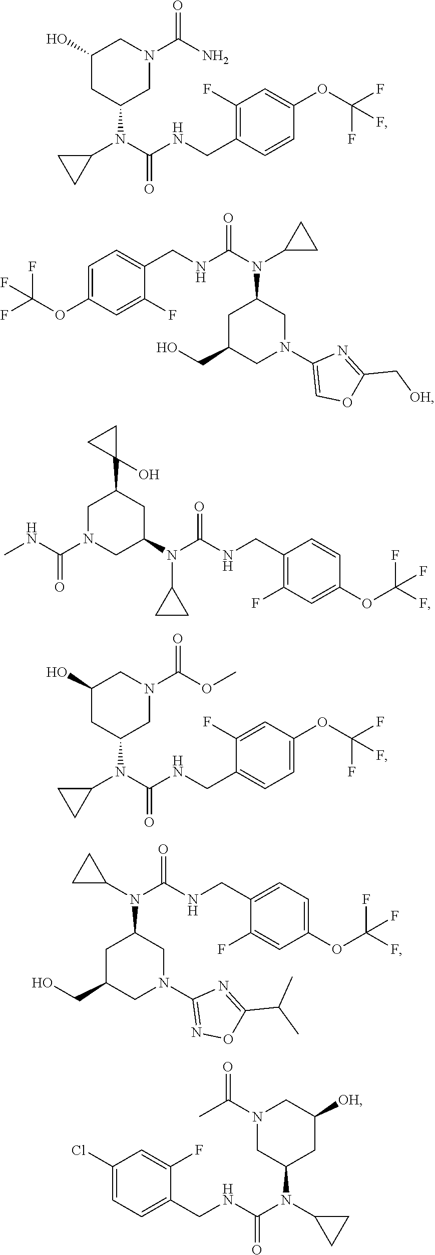

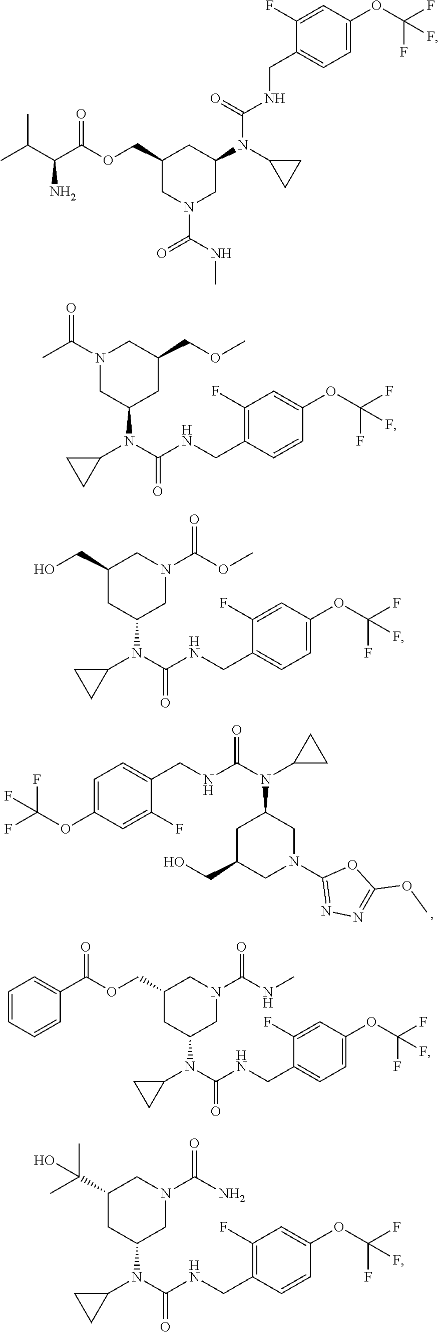

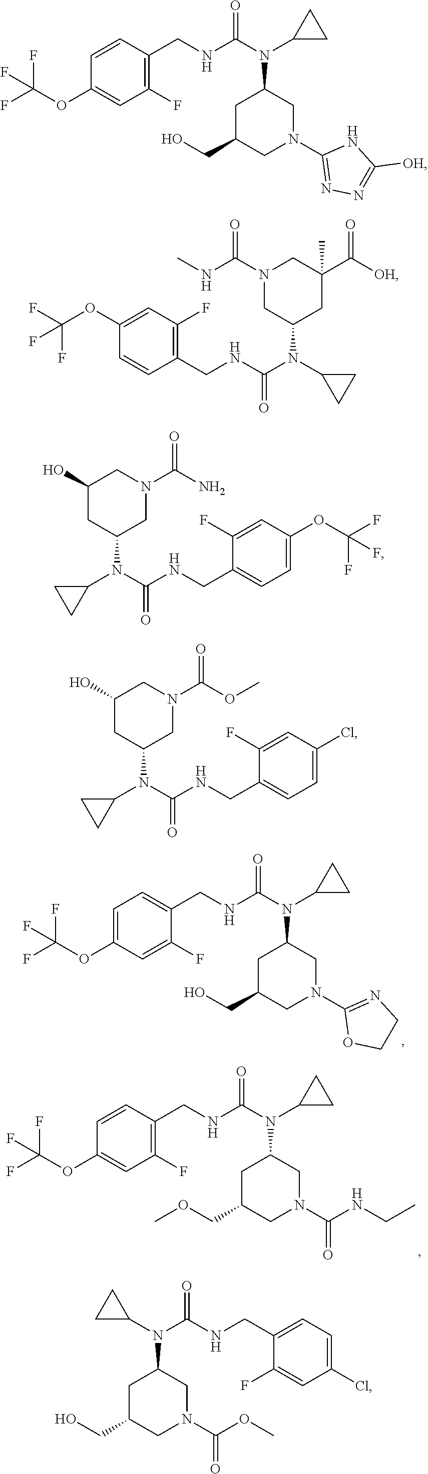

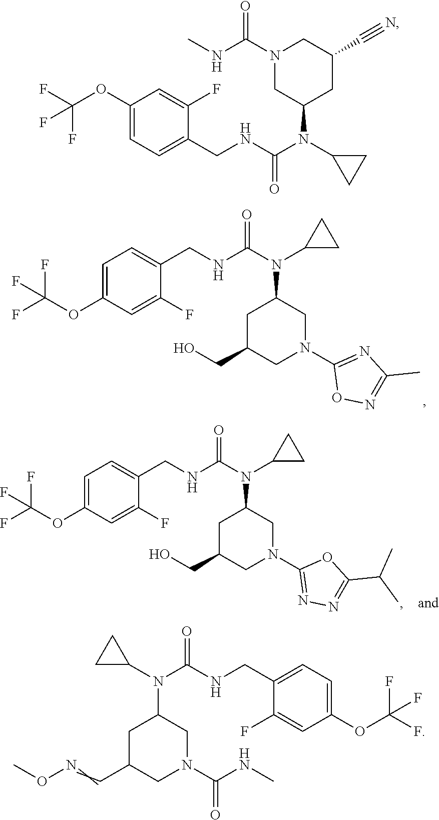

In certain embodiments, a compound or a pharmaceutically acceptable salt thereof having the structure:

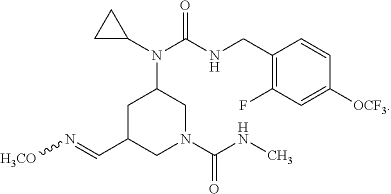 [SMILES: CNC(=O)N1CC(C=NOC)CC(N(C(=O)NCc2ccc(OC(F)(F)F)cc2F)C2CC2)C1]

### Exemplary Compounds of Formula (III)

In certain embodiments, a compound or a pharmaceutically acceptable salt thereof having the structure of any one of the following compounds:

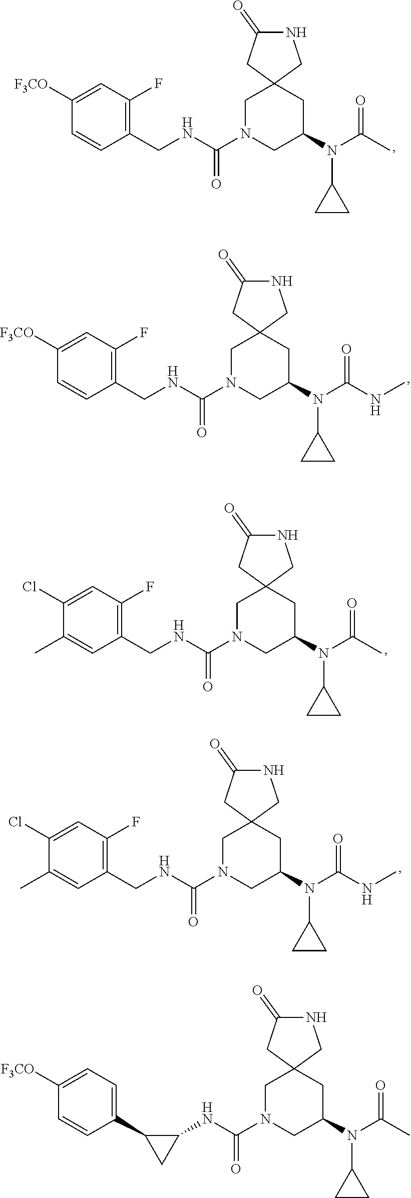 [SMILES: CC(=O)N(C1CC1)[C@H]1CN(C(=O)NCc2ccc(OC(F)(F)F)cc2F)CC2(CNC(=O)C2)C1 ; CNC(=O)N(C1CC1)[C@H]1CN(C(=O)NCc2ccc(OC(F)(F)F)cc2F)CC2(CNC(=O)C2)C1 ; CC(=O)N(C1CC1)[C@H]1CN(C(=O)NCc2cc(C)c(Cl)cc2F)CC2(CNC(=O)C2)C1 ; CNC(=O)N(C1CC1)[C@H]1CN(C(=O)NCc2cc(C)c(Cl)cc2F)CC2(CNC(=O)C2)C1 ; CC(=O)N(C1CC1)[C@H]1CN(C(=O)N[C@@H]2C[C@H]2c2ccc(OC(F)(F)F)cc2)CC2(CNC(=O)C2)C1]

(prepared from a racemic mixture of trans-cyclopropyl stereoisomers),

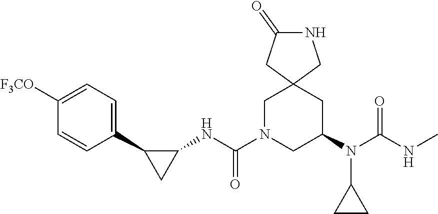 [SMILES: CNC(=O)N(C1CC1)[C@H]1CN(C(=O)N[C@@H]2C[C@H]2c2ccc(OC(F)(F)F)cc2)CC2(CNC(=O)C2)C1]

(prepared from a racemic mixture of trans-cyclopropyl stereoisomers),

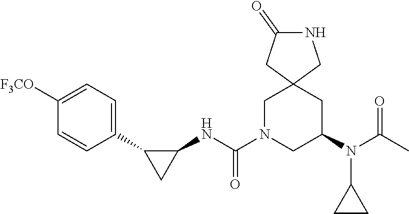 [SMILES: CC(=O)N(C1CC1)[C@H]1CN(C(=O)N[C@H]2C[C@@H]2c2ccc(OC(F)(F)F)cc2)CC2(CNC(=O)C2)C1]

(prepared from a racemic mixture of trans-cyclopropyl stereoisomers),

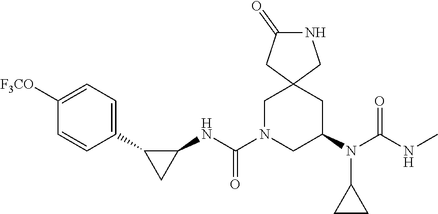 [SMILES: CNC(=O)N(C1CC1)[C@H]1CN(C(=O)N[C@H]2C[C@@H]2c2ccc(OC(F)(F)F)cc2)CC2(CNC(=O)C2)C1]

(prepared from a racemic mixture of trans-cyclopropyl stereoisomers),

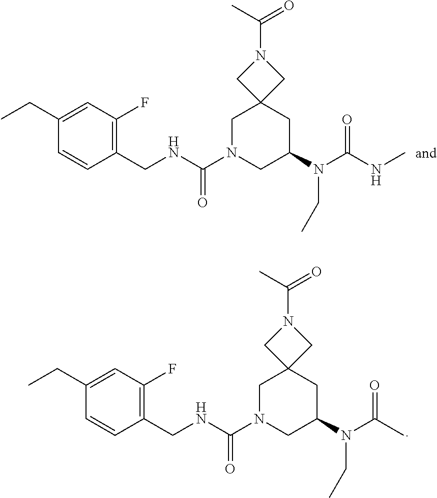

### Exemplary Compounds of Formula (IV)

In certain embodiments, a compound or a pharmaceutically acceptable salt thereof having the structure of any one of the following compounds:

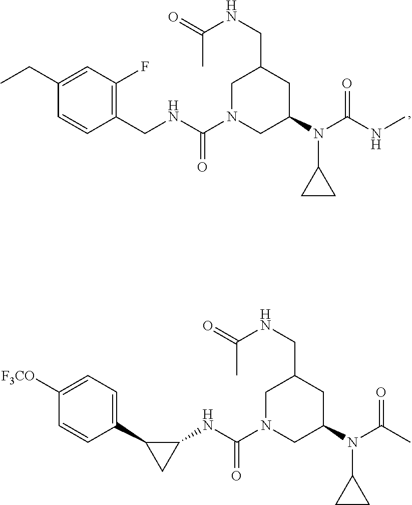 [SMILES: CCc1ccc(CNC(=O)N2CC(CNC(C)=O)C[C@@H](N(C(=O)NC)C3CC3)C2)c(F)c1 ; CC(=O)NCC1C[C@@H](N(C(C)=O)C2CC2)CN(C(=O)N[C@@H]2C[C@H]2c2ccc(OC(F)(F)F)cc2)C1]

(prepared from a racemic mixture of trans-cyclopropyl stereoisomers),

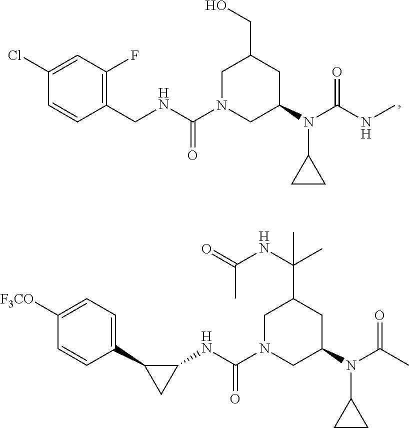 [SMILES: CNC(=O)N(C1CC1)[C@@H]1CC(CO)CN(C(=O)NCc2ccc(Cl)cc2F)C1 ; CC(=O)NC(C)(C)C1C[C@@H](N(C(C)=O)C2CC2)CN(C(=O)N[C@@H]2C[C@H]2c2ccc(OC(F)(F)F)cc2)C1]

(prepared from a racemic mixture of trans-cyclopropyl stereoisomers),

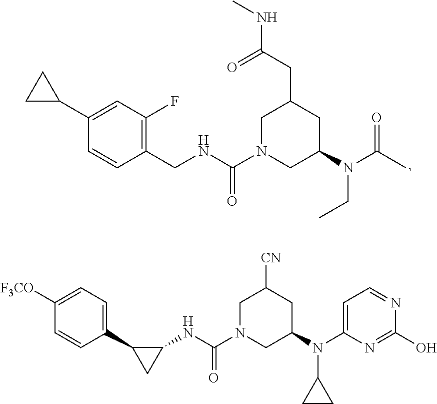 [SMILES: CCN(C(C)=O)[C@@H]1CC(CC(=O)NC)CN(C(=O)NCc2ccc(C3CC3)cc2F)C1 ; N#CC1C[C@@H](N(c2ccnc(O)n2)C2CC2)CN(C(=O)N[C@@H]2C[C@H]2c2ccc(OC(F)(F)F)cc2)C1]

(prepared from a racemic mixture of trans-cyclopropyl stereoisomers),

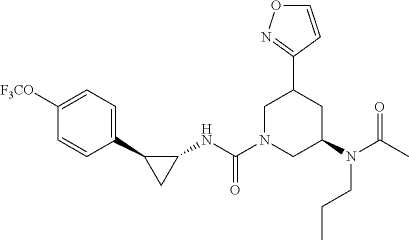 [SMILES: CCCN(C(C)=O)[C@@H]1CC(c2ccon2)CN(C(=O)N[C@@H]2C[C@H]2c2ccc(OC(F)(F)F)cc2)C1]

(prepared from a racemic mixture of trans-cyclopropyl stereoisomers), and

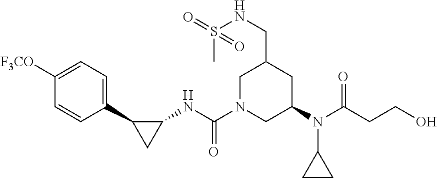 [SMILES: CS(=O)(=O)NCC1C[C@@H](N(C(=O)CCO)C2CC2)CN(C(=O)N[C@@H]2C[C@H]2c2ccc(OC(F)(F)F)cc2)C1]

(prepared from a racemic mixture of trans-cyclopropyl stereoisomers).

In certain embodiments, the compound is selected from the structure of any one of the compounds recited in Table 1, 2 or 3 (recited in Example 2).

In some embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., —CD₃, —OCD₃).

Any compound of the invention can also be radiolabeled for the preparation of a radiopharmaceutical agent.

### Methods of Treatment

One aspect of the invention provides compounds, compositions, and methods useful for treating or preventing a disease or disorder associated with abnormal levels of amino acids by modulation of SLC6A19 transport.

Another aspect of the invention relates to methods of modulating SLC6A19 transport in a subject in need thereof comprising administering to the subject an effective amount of a compound of Formula (I), (II), (III), or (IV).

Another aspect of the invention relates to methods of treating or preventing a disease or disorder associated with a genetic defect in phenylalanine hydroxylase in a subject in need thereof comprising administering to the subject an effective amount of a compound of Formula (I), (II), (III), or (IV).

In some embodiments, the invention relates to methods of treating or preventing phenylketonuria in a subject in need thereof comprising administering to the subject an effective amount of a compound of Formula (I), (II), (III), or (IV).

In some embodiments, the invention relates to methods of treating or preventing hyperphenylalaninemia in a subject in need thereof comprising administering to the subject an effective amount of a compound of Formula (I), (II), (III), or (IV).

In some embodiments, the compound reduces systemic phenylalanine levels in the subject.

In some embodiments, the invention relates to methods of treating or preventing tyrosinemia (Type I, II, or III) in a subject in need thereof comprising administering to the subject an effective amount of a compound of Formula (I), (II), (III), or (IV).

In some embodiments, the compound reduces systemic glycine levels in the subject.

In some embodiments, the invention relates to methods of treating or preventing isovaleric acidemia, methylmalonic acidemia, propionic acidemia, maple syrup urine disease, DNAJC12 deficiency, urea cycle disorders, or hyperammonemia in a subject in need thereof comprising administering to the subject an effective amount of a compound of Formula (I), (II), (III), or (IV).

In some embodiments of any one of the disclosed methods, the compound modulates SLC6A19 in the subject.

In some embodiments of any one of the disclosed methods, the compound inhibits SLC6A19 in the subject.

In some embodiments of any one of the disclosed methods, the compound modulates SLC6A19 transport in the subject.

In some embodiments of any one of the disclosed methods, the compound inhibits SLC6A19 transport in the subject.

In some embodiments, the compound reduces systemic levels of an amino acid in the subject.

In some embodiments of any one of the disclosed methods, wherein the subject is a mammal. In some embodiments of any one of the disclosed methods, the mammal is a human.

In some embodiments of any one of the disclosed methods, the compound of Formula (I).

In some embodiments of any one of the disclosed methods, the compound of Formula (II).

In some embodiments of any one of the disclosed methods, the compound of Formula (III).

In some embodiments of any one of the disclosed methods, the compound of Formula (IV).

In some embodiments of any one of the disclosed methods, the compound is selected from the structure of any one of the compounds recited in Table 1.

In some embodiments of any one of the disclosed methods, the compound is selected from the structure of any one of the compounds recited in Table 2.

In some embodiments of any one of the disclosed methods, the compound is selected from the structure of any one of the compounds recited in Table 3.

### Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appi Biochem 4:185-9 (1982). Other polymers that could be used are poly-1,3-dioxolane and poly-1,3,6-trioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5):143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethane, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodable, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### Example 1: SLC6A19 Isoleucine Transport Assay

### Cell Line Generation and Maintenance

The Flp-In™ T-REx™ 293 cell line was purchased from Thermo Fisher Scientific. The line was used to generate a stable cell line inducibly expressing human SLC6A19 with a C-terminal V5 tag and stably expressing human TMEM27 (also known as Collectrin) with a C-terminal myc-DDK tag. The stable cell line was generated by transfecting SLC6A19- and TMEM27-encoding plasmids using standard protocols, followed by antibiotic selection. Stable cells were maintained in DMEM/F12 supplemented with Glutamax, 10% fetal bovine serum, 100 U/mL penicillin, 100 ug/mL streptomycin, 200 ug/mL hygromycin, 10 ug/mL blasticidin and 300 ug/mL neomycin (Thermo Fisher).

### Assay: Isoleucine Transport Assay in 96-Well Format

Stable cell lines were seeded at a density of 35,000 cells per well in a poly-D-lysine coated 96-well cell culture-treated plate on day 0. On day 1 the expression of SLC6A19 was induced by dispensing tetracycline at a final concentration of 1 ug/mL using a Tecan D300e digital dispenser. On day 2 the transport assay was run. Media was removed from the plate using the GentleSpin setting of a Centrifugal Blue Washer (Blue Cat Bio) and cells were washed with 175 uL live cell imaging solution (Thermo Fisher) using the Blue Washer. Following washing, cells were treated with 70 μL of either DMSO, positive control or compound, diluted in Krebs buffer (140 mM NaCl, 4.7 mM KCl, 2.5 mM CaCl₂, 1.2 mM MgCl₂, 11 mM HEPES, 10 mM Glucose, pH 7.4) at room temperature. After 20-60 minutes 30 uL of a 3.3 mM solution of 13C6,15N-L-isoleucine (Cambridge Isotope Laboratories) was added. After 20 min incubation with the isoleucine substrate at room temperature cells were washed with 175 uL live cell imaging solution using the Blue Washer. Cells were then lysed in 150 uL of 15 uM D-Leucine-d10 (CDN Isotopes) in ultrapure water. Plates were put on a shaker at 700 rpm for a minimum of 40 minutes to facilitate lysis. Following lysis, a standard dilution curve of 13C6,15N-L-isoleucine was added to wells containing lysates of untreated cells. Plates were returned to the shaker for a minimum of 2 minutes to ensure proper mixing of the standard curve. Plates were then centrifuged for 5 min at 4,000 rpm to pellet cellular debris and precipitate. Supernatants were diluted 1:10 in acetonitrile+0.1% formic acid in polypropylene plates.

### Assay: Isoleucine Transport Assay in 384-Well Format

On day 0, stable cell lines were seeded at a density of 20,000 cells per well in a poly-D-lysine coated 384-well cell culture-treated plate in media containing 1 ug/mL tetracycline using a Viaflo 384-well pipette. Transport assays were run the following day (day 1). Media was removed from the plate using the GentleSpin setting of a Centrifugal Blue Washer (Blue Cat Bio) and cells were washed with 80 uL live cell imaging solution (Thermo Fisher) using the Blue Washer. Following washing, cells were treated with 20 uL of either DMSO, positive control or compound, diluted in Krebs buffer (140 mM NaCl, 4.7 mM KCl, 2.5 mM CaCl₂), 1.2 mM MgCl₂, 11 mM HEPES, 10 mM Glucose, pH 7.4) using a TECAN liquid handler. After 20-60 minutes incubation at room temperature 8.6 uL of a 3.3 mM solution of 13C6,15N-L-isoleucine (Cambridge Isotope Laboratories) was added. After 20 min incubation with the isoleucine substrate at room temperature cells were washed with 80 uL live cell imaging solution using the Blue Washer. Cells were then lysed in 80 uL of 15 uM D-Leucine-d₁₀ (CDN Isotopes) in ultrapure water. Plates were put on a shaker at 700 rpm for a minimum of 2 hours to facilitate lysis. Following lysis, a standard dilution curve of ¹³C₆,¹⁵N-L-isoleucine was added to wells containing lysates of untreated cells. Plates were returned to the shaker for a minimum of 5 minutes to ensure proper mixing of the standard curve. Plates were then centrifuged for 10 min at 4,000 rpm to pellet cellular debris and precipitate. Supernatants were diluted 1:10 in acetonitrile+0.1% formic acid in polypropylene plates.

¹³C₆,¹⁵N-L-isoleucine analysis was performed using a RapidFire365-QTOF 6545 (Agilent). Quantitative sample analysis utilizes automated solid-phase extraction (HILIC H6 cartridge) prior to mass spec injection. Samples were loaded using 95% acetonitrile, 0.1% formic acid and eluted from the cartridge with 5% acetonitrile, 0.1% formic acid directly for ESI-MS (electrospray ionization) analysis. Quantification of the analytes were performed using Agilent Masshunter Quant software from the high-resolution full scan data.

### Example 2: Synthesis of Exemplary Compounds

### Procedure 1: Synthesis of 2-fluoro-1-(isocyanatomethyl)-4-(trifluoromethoxy)benzene

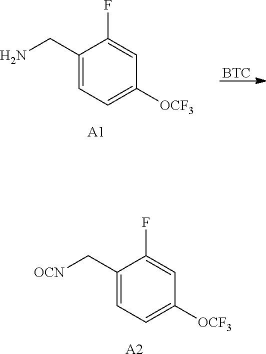

To a solution of A1 (78 mg, 0.37 mmol) in toluene (3 mL) was added a solution of triphosgene (53 mg, 0.18 mmol) in toluene (1 mL) at 0° C. The resulting mixture was stirred at 120° C. for 2 hrs under N₂ atmosphere. After cooling to room temperature, the mixture was concentrated under reduced pressure to give crude A2 (87 mg, 99.20% yield) as a yellow oil which was used directly in the next step without further purification.

### Procedure 2: Synthesis of N-(2-fluoro-4-(trifluoromethoxy)benzyl)-1H-imidazole-1-carboxamide

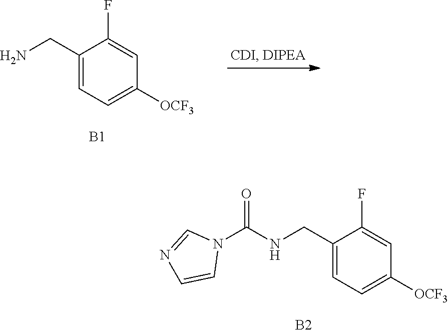

To a solution of CDI (426 mg, 2.63 mmol) and diisopropylethylamine (833 μL, 4.78 mmol) in DMF (3.95 mL) was added A1 (500 mg, 2.39 mmol) portion wise. The mixture was stirred at room temp for 2 hr at which time LCMS indicated the complete consumption of the starting material. The crude solution of B2 (0.5 M) was directly used for the next step. LC/MS (ESI) m/z: 304 (M+H)⁺.

### Procedure 3: Synthesis of (5R,9R)-9-(1-cyclopropyl-3-(2-fluoro-4-(trifluoromethoxy)benzyl)ureido)-N-methyl-3-oxo-2,7-diazaspiro[4.5]decane-7-carboxamide (C14-1) and (5S,9R)-9-(1-cyclopropyl-3-(2-fluoro-4-(trifluoromethoxy)benzyl)ureido)-N-methyl-3-oxo-2,7-diazaspiro[4.5]decane-7-carboxamide (C14-2)

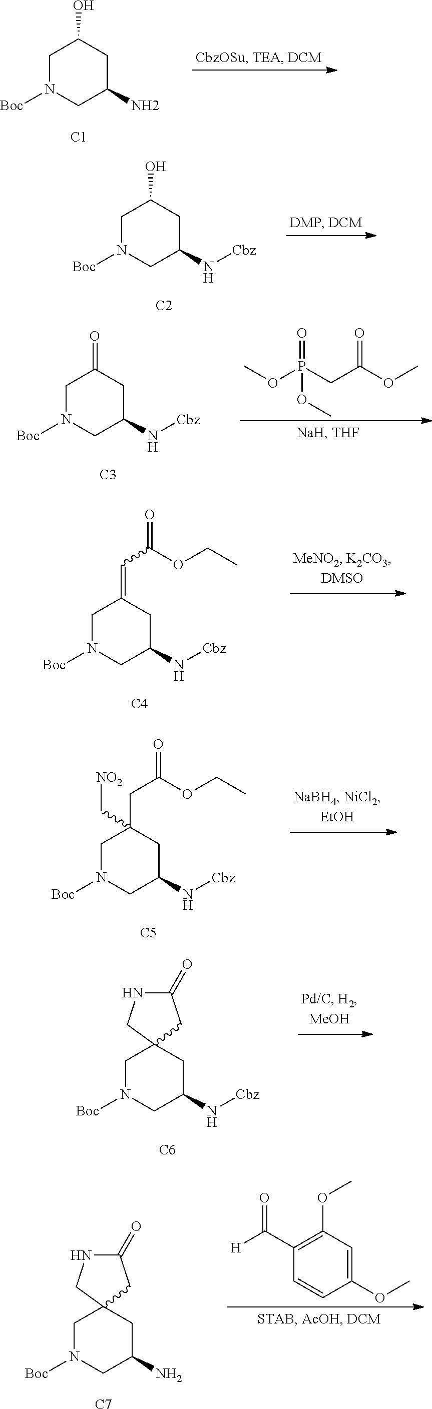

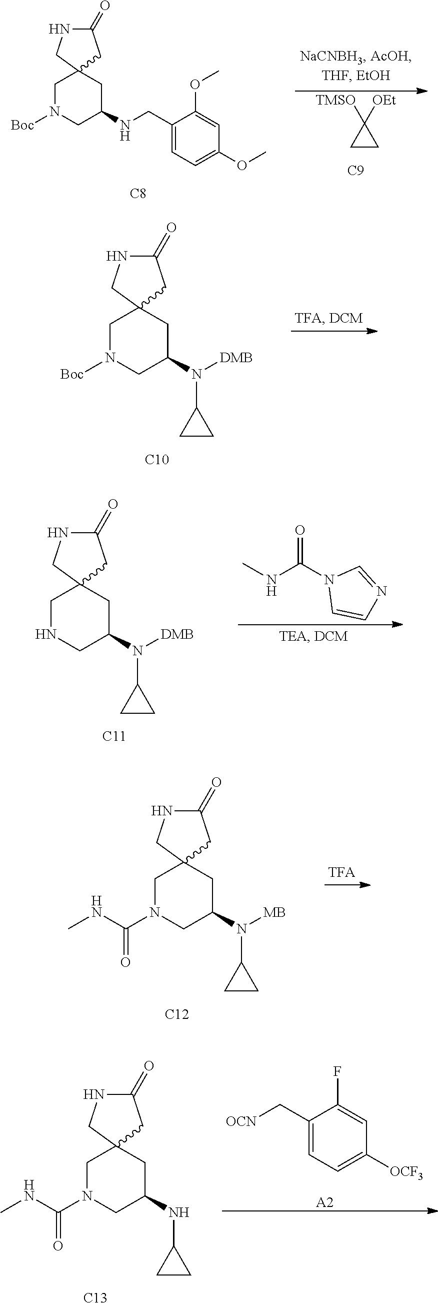

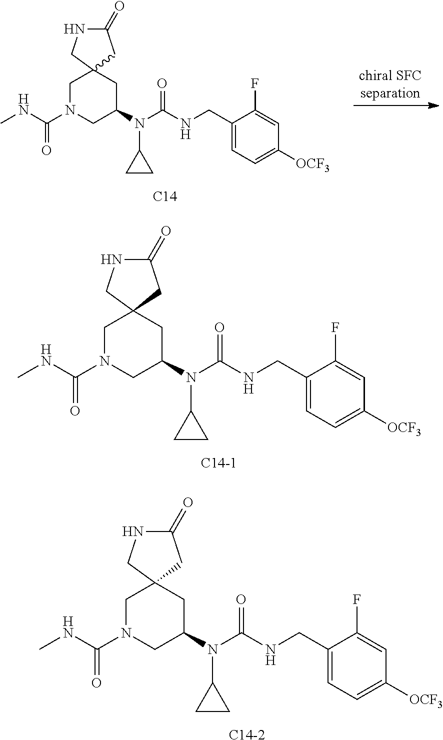 [SMILES: CNC(=O)N1C[C@H](N(C(=O)NCc2ccc(OC(F)(F)F)cc2F)C2CC2)CC2(CNC(=O)C2)C1 ; "C14" ; CNC(=O)N1C[C@H](N(C(=O)NCc2ccc(OC(F)(F)F)cc2F)C2CC2)C[C@@]2(CNC(=O)C2)C1 ; "C14-1" ; CNC(=O)N1C[C@H](N(C(=O)NCc2ccc(OC(F)(F)F)cc2F)C2CC2)C[C@]2(CNC(=O)C2)C1 ; "C14-2"]

Synthesis of C2: To a mixture of C1 (1.0 g, 4.62 mmol) and TEA (1.17 g, 11.56 mmol) in DCM (15 mL) was added Cbz-OSu (1.73 g, 6.94 mmol) at 0° C., the resulting mixture was stirred at room temperature for 20 hrs. Then the mixture was diluted with H₂O (40 mL) and extracted with DCM (30 mL) twice. The combined organic layers were washed with brine (40 mL), dried over anhydrous Na₂SO₄ and concentrated to dryness. The residue was purified by column chromatography on silica gel (eluted with DCM:MeOH=100:0 to 100:3) to give C2 (1.59 g, 98.14% yield) as white solid. LC/MS (ESI) m/z: 295 (M+H−56)⁺.

Synthesis of C3: To a solution of C2 (800 mg, 2.28 mmol) in DCM (10 mL) was added Dess-Martin periodinane (1.94 g, 4.57 mmol) at 0° C., the resulting mixture was stirred at room temperature for 2 hrs. Then the mixture was quenched with saturated NaHCO₃ solution (30 mL) and extracted with DCM (20 mL) twice. The combined organic layers were washed with brine (30 mL), dried over anhydrous Na₂SO₄ and concentrated to dryness. The residue was purified by column chromatography on silica gel (eluted with DCM:MeOH=100:0 to 50:1) to give C3 (789 mg, 99.20% yield) as light-yellow oil. LC/MS (ESI) m/z: 249 (M+H−100)⁺.

Synthesis of C4: To a mixture of trimethyl phosphonoacetate (496 mg, 2.72 mmol) in THF (12 mL) was added NaH (109 mg, 2.72 mmol) at 0° C. under N₂ atmosphere, the mixture was stirred at 0° C. for 30 mins. Then a solution of C3 (789 mg, 2.27 mmol) in THF (6 mL) was added into the above mixture at 0° C., the resulting mixture was stirred at room temperature for another 16 hrs. Then the mixture was quenched with saturated NH₄Cl solution (40 mL) and extracted with EtOAc (25 mL×2). The combined organic layers were washed with brine (30 mL), dried over anhydrous Na₂SO₄, filtered and concentrated to dryness. The residue was purified by column chromatography on silica gel (eluted with PE:EtOAc=100:0 to 3:1) to give C4 (733 mg, 80.03% yield) as colorless oil. LC/MS (ESI) m/z: 305 (M+H−100)⁺.

Synthesis of 5: To a mixture of C4 (718 mg, 1.78 mmol) and K₂CO₃ (246 mg, 1.78 mmol) in DMSO (16 mL) was added nitromethane (1.08 g, 17.77 mmol), the resulting mixture was stirred at 100° C. for 16 hrs under N₂ atmosphere. Then the mixture was diluted with H₂O (40 mL) and extracted with EtOAc (25 mL×2). The combined organic layers were washed with brine (40 mL), dried over anhydrous Na₂SO₄, filtered and concentrated to dryness. The residue was purified by column chromatography on silica gel (eluted with PE:EtOAc=100:0 to 4:1) to give C5 (384 mg, 46.47% yield) as colorless oil. LC/MS (ESI) m/z: 366 (M+H−100)⁺.

Synthesis of C6: To a solution of C5 (384 mg, 0.80 mmol) in EtOH (15 mL) was added nickel chloride hexahydrate (1.04 g, 8.01 mmol) and NaBH₄ (304 mg, 8.01 mmol) at 0° C., the resulting mixture was stirred at room temperature for 2 hrs. Then the mixture was quenched with saturated NH₄Cl solution (30 mL) and extracted with EtOAc (25 mL×2). The combined organic layers were washed with brine (30 mL), dried over anhydrous Na₂SO₄, filtered and concentrated to dryness. The residue was purified by column chromatography on silica gel (eluted with DCM:MeOH=100:0 to 25:1) to give C6 (221 mg, 68.40% yield) as colorless oil. LC/MS (ESI) m/z: 304 (M+H−100)⁺.

Synthesis of C7: To a solution of C6 (221 mg, 0.55 mmol) in MeOH (10 mL) was added Pd/C (200 mg, 10% w/w), the resulting mixture was degassed under N₂ atmosphere for three times and stirred at room temperature for 2 hrs under H₂ atmosphere. Then the mixture was filtered and the filtrate was concentrated to give crude C7 (147 mg, 99.64% yield) as colorless oil which was used at the next step directly without further purification. LC/MS (ESI) m/z: 270 (M+H)⁺.

Synthesis of C8: To a mixture of C7 (147 mg, 0.55 mmol) and 2,4-dimethoxybenzaldehyde (91 mg, 0.55 mmol) in DCM (8 mL) was added AcOH (65 mg, 1.09 mmol) and the mixture was stirred at room temperature for 1 hr. Then NaBH(OAc)₃ (348 mg, 1.64 mmol) was added at 0° C. and the resulting mixture was stirred at room temperature for 16 hrs.

After concentration, the residue was purified by column chromatography on silica gel (eluted with DCM:MeOH=100:0 to 12:1) to give C8 (228 mg, 99.58% yield) as colorless oil. LC/MS (ESI) m/z: 420 (M+H)⁺.

Synthesis of C10: To a mixture of C8 (228 mg, 0.54 mmol) and C9 (238 mg, 1.36 mmol) in EtOH (5 mL) and THF (10 mL) was added AcOH (327 mg, 5.44 mmol) and NaBH₃CN (120 mg, 1.90 mmol). The resulting mixture was stirred at 80° C. for 4 hrs under N₂ atmosphere. Then the mixture was concentrated to dryness, the residue was dissolved in EtOAc (30 mL) and washed with saturated NaHCO₃ solution (30 mL). The organic layer was separated, washed with brine (30 mL), dried over anhydrous Na₂SO₄, filtered and concentrated to dryness. The residue was purified by column chromatography on silica gel (eluted with DCM:MeOH=100:0 to 20:1) to give C10 (210 mg, 84.08% yield) as colorless oil. LC/MS (ESI) m/z: 460 (M+H)⁺.

Synthesis of C11: To a solution of C10 (210 mg, 0.46 mmol) in DCM (8 mL) was added TFA (2 mL) dropwise at 0° C. and the resulting mixture was stirred at room temperature for 1 hr. LCMS indicated the complete consumption of the starting material. Then the mixture was concentrated to give crude C11 (161 mg, 98.02% yield) as colorless oil which was used at the next step directly without further purification. LC/MS (ESI) m/z: 360 (M+H)⁺.

Synthesis of C12: To a mixture of C11 (161 mg, 0.45 mmol) and DIEA (271 mg, 2.08 mmol) in MeCN (10 mL) was added N-methyl-1H-imidazole-1-carboxamide (325 mg, 2.60 mmol) and the resulting mixture was stirred at 60° C. for 16 hrs. Then the mixture was diluted with H₂O (30 mL) and extracted with EtOAc (20 mL×2), the combined organic layers were washed with brine (30 mL), dried over anhydrous Na₂SO₄, filtered and concentrated to dryness. The residue was purified by column chromatography on silica gel (eluted with DCM:MeOH=100:0 to 12:1) to give C12 (168 mg, 90.05% yield) as colorless oil. LC/MS (ESI) m/z: 417 (M+H)⁺.

Synthesis of C13: A round-bottom flask was charged with C12 (168 mg, 0.40 mmol) and TFA (5 mL), the mixture was stirred at 80° C. for 4 hrs. LCMS indicated the complete consumption of the starting material. Then the mixture was concentrated to give crude C13 (98 mg, 91.23% yield) as purple oil which was used at the next step directly without further purification. LC/MS (ESI) m/z: 267 (M+H)⁺.

Synthesis of C14-1 and C14-2: To a mixture of C13 (98 mg, 0.37 mmol) and TEA (0.2 mL, 1.07 mmol) in DCM (6 mL) was added a solution of A2 (87 mg, 0.37 mmol) in DCM (2 mL) at 0° C., the resulting mixture was stirred at room temperature for 30 mins. Then the mixture was diluted with H₂O (30 mL) and extracted with DCM (20 mL×2). The combined organic layers were washed with brine (30 mL), dried over anhydrous Na₂SO₄, filtered and concentrated to dryness. The residue was purified by column chromatography on silica gel (eluted with DCM:MeOH=100:0 to 8:1) to give pure C14 (42 mg, 18.88% yield) as white solid. LC/MS (ESI) m/z: 502 (M+H)⁺. This was further purified via SFC separation (SHIMADZU prep solution SFC, ChiralCel OZ, 250×21.2 mm I.D., 5 μm) to give C14-1 (6.4 mg, 3.47% yield) as white solid. ¹H NMR (400 MHz, MeOD) δ 7.50-7.41 (m, 1H), 7.17-7.05 (m, 2H), 4.52-4.39 (m, 2H), 4.15-4.03 (m, 1H), 3.97-3.87 (m, 1H), 3.83-3.70 (m, 1H), 3.32-3.30 (m, 1H), 3.25-3.18 (m, 1H), 3.17-3.07 (m, 1H), 2.72 (s, 3H), 2.64-2.57 (m, 1H), 2.56-2.49 (m, 1H), 2.33-2.21 (m, 2H), 2.19-2.10 (m, 1H), 2.00-1.92 (m, 1H), 1.02-0.94 (m, 2H), 0.83-0.72 (m, 2H); ¹⁹F NMR (377 MHz, MeOD) δ −59.76 (s), −116.95 (s); and C14-2 (6.5 mg, 3.52% yield) as white solid. ¹H NMR (400 MHz, MeOD) δ 7.49-7.41 (m, 1H), 7.16-7.07 (m, 2H), 4.51-4.39 (m, 2H), 4.09-4.02 (m, 1H), 3.97-3.88 (m, 1H), 3.80-3.67 (m, 1H), 3.22-3.14 (m, 3H), 2.72 (s, 3H), 2.66-2.60 (m, 1H), 2.59-2.53 (m, 1H), 2.38-2.31 (m, 1H), 2.30-2.21 (m, 2H), 1.94-1.86 (m, 1H), 1.02-0.95 (m, 2H), 0.82-0.75 (m, 2H); ¹⁹F NMR (377 MHz, MeOD) δ −59.77 (s), −116.97 (s).

### Procedure 4: Synthesis of (9R)-9-(1-cyclopropyl-3-((1S,2R)-2-(4-(trifluoromethoxy)phenyl)cyclopropyl)ureido)-N-methyl-3-oxo-2,7-diazaspiro[4.5]decane-7-carboxamide (D5)

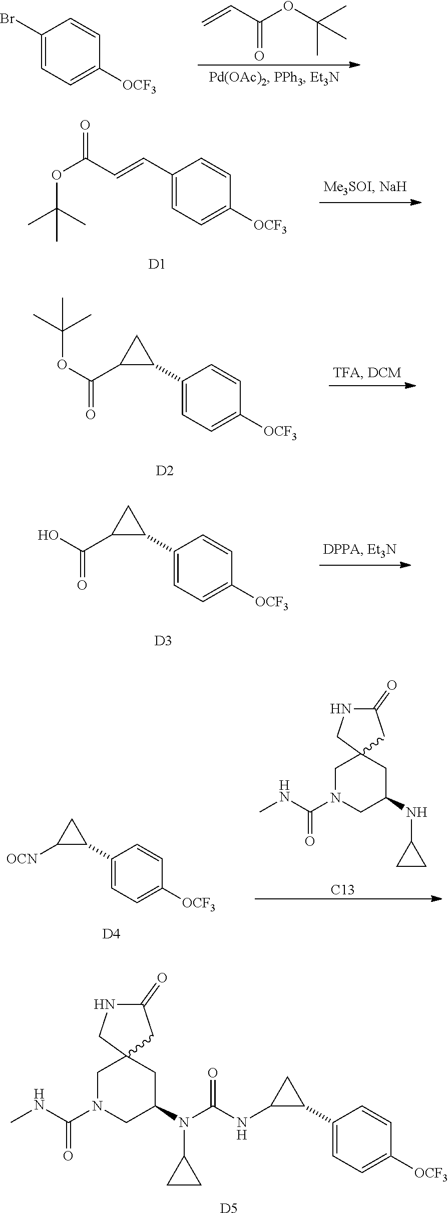

Synthesis of D1: To a mixture of 1-bromo-4-(trifluoromethoxy)benzene (5.00 g, 20.75 mmol) and t-butyl acrylate (3.99 g, 31.12 mmol) in DMF (70 mL) was added TEA (20 mL), PPh₃ (544 mg, 2.07 mmol) and Pd(OAc)₂ (466 mg, 2.07 mmol). The resulting mixture was stirred at 80° C. for 8 hours under N₂ atmosphere. Then the mixture was diluted with EtOAc (100 mL), filtered and the filtrate was washed with saturated NH₄Cl solution (80 mL) twice. The organic layer was separated, washed with brine (100 mL), dried over anhydrous Na₂SO₄, filtered and concentrated to dryness. The residue was purified via column chromatography on silica gel (eluted with PE:EtOAc=100:0 to 30:1) to give D1 (3.80 g, 63.54% yield) as colorless oil.

Synthesis of D2: To a solution of trimethylsulfoxonium iodide (4.35 g, 19.77 mmol) in anhydrous DMSO (50 m L) at 0° C. was added NaH (791 mg, 19.77 mmol, 60% dispersion in Paraffin Liquid) in portions. After stirring at 0° C. for 30 mins, a solution of D1 (3.80 g, 13.18 mmol) in DMSO (30 mL) was added into the above mixture. The resulting mixture was stirred at r.t. for 4 hrs and then the mixture was quenched with saturated NH₄Cl solution (120 mL) and extracted with EtOAc (70 mL) twice. The combined organic layers were washed with water (100 mL) and brine (100 mL), dried over MgSO₄, filtered and concentrated to dryness. The crude product was purified by column chromatography on silica gel (eluted with PE:EtOAc=100:0 to 30:1) to give D2 (2.10 g, 52.70% yield) as colorless oil.

Synthesis of D3: To a solution of D2 (2.10 g, 6.95 mmol) in DCM (30 mL) was added TFA (10 mL) at 0° C. dropwise under N₂ atmosphere. The resulting mixture was stirred at room temperature for 2 hrs. Then the mixture was concentrated under reduced pressure to give crude D3 (1.71 g, 99.97% yield) as yellow oil which was used at the next step directly without further purification. LC/MS (ESI) m/z: 245 (M−H)⁻.

Synthesis of D4: To a mixture of D3 (39 mg, 0.16 mmol) and TEA (32 mg, 0.32 mmol) in toluene (4 mL) was added DPPA (53 mg, 0.19 mmol) dropwise at 0° C. The resulting mixture was stirred at 120° C. for 3 hrs. After cooling to room temperature, the mixture was concentrated under reduced pressure to give crude D4 (38 mg, 98.64% yield) as colorless oil which was used at the next step directly without further purification.

Synthesis of D5: To a solution of C13 (42 mg, 0.16 mmol) in DCM (4 mL) were added TEA (162 mg, 1.60 mmol) and a solution of D4 (38 mg, 0.16 mmol) in DCM (2 mL) at 0° C., the resulting mixture was stirred at room temperature for 30 mins. Then the mixture was diluted with H₂O (20 mL) and extracted with DCM (20 mL) twice. The combined organic layers were separated, washed with brine (20 mL), dried over anhydrous Na₂SO₄, filtered and concentrated to dryness under reduced pressure. The residue was purified via prep-HPLC to give D5 (17.7 mg, 22.02% yield) as white solid. LC/MS (ESI) m/z: 510 (M+H)⁺. ¹H NMR (400 MHz, MeOD) δ 7.27 (d, J=8.1 Hz, 2H), 7.18 (d, J=8.4 Hz, 2H), 6.75 (s, 1H), 4.12-4.00 (m, 1H), 3.99-3.87 (m, 1H), 3.80-3.66 (m, 1H), 3.32-3.30 (m, 0.5H), 3.24-3.20 (m, 0.5H), 3.20-3.18 (m, 1H), 3.17-3.08 (m, 1H), 2.80-2.74 (m, 1H), 2.72 (s, 3H), 2.67-2.58 (m, 1H), 2.53-2.44 (m, 1H), 2.39-2.31 (m, 0.5H), 2.31-2.20 (m, 2H), 2.17-2.11 (m, 0.5H), 2.11-2.03 (m, 1H), 1.99-1.87 (m, 1H), 1.31-1.19 (m, 2H), 1.02-0.89 (m, 2H), 0.82-0.69 (m, 2H). ¹⁹F NMR (376 MHz, MeOD) δ −59.62 (d, J=4.4 Hz).

### Procedure 5: Synthesis of (5R,9R)-9-(1-cyclopropyl-3-((3-(3-(trifluoromethoxy) phenyl)isoxazol-5-yl)methyl)ureido)-N-methyl-3-oxo-2,7-diazaspiro[4.5]decane-7-carboxamide (E5-1) and (5S,9R)-9-(1-cyclopropyl-3-((3-(3-(trifluoromethoxy)phenyl)isoxazol-5-yl)methyl)ureido)-N-methyl-3-oxo-2,7-diazaspiro[4.5]decane-7-carboxamide (E5-2)

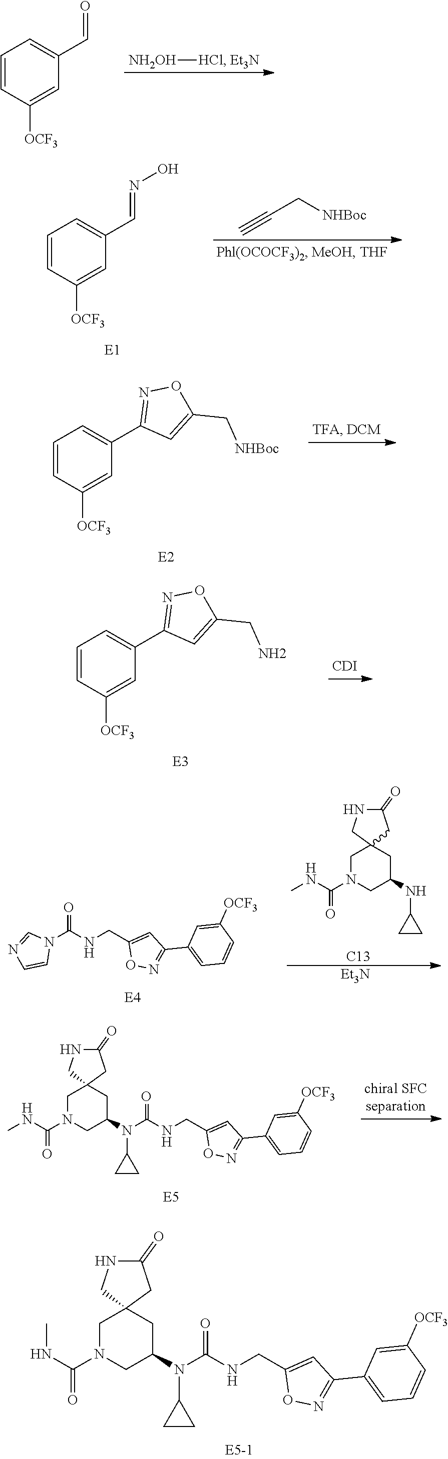

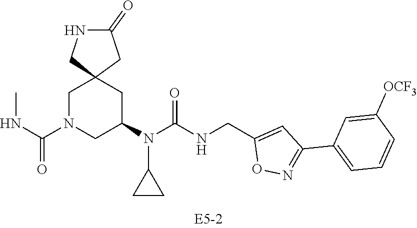 [SMILES: CNC(=O)N1C[C@H](N(C(=O)NCc2cc(-c3cccc(OC(F)(F)F)c3)no2)C2CC2)C[C@]2(CNC(=O)C2)C1]

Synthesis of E1: To a solution of 3-(trifluoromethoxy)benzaldehyde (1.0 g, 5.26 mmol) in PGP-DCM (25 mL) were added TEA (586 mg, 5.79 mmol) and hydroxylamine hydrochloride (402 mg, 5.79 mmol) at 0° C. The resulting mixture was stirred at room temperature for 4 hours. Then the mixture was diluted with water (50 mL) and extracted with DCM (30 mL) twice.

The combined organic layers were separated, washed with brine (30 mL), dried over anhydrous Na₂SO₄, filtered and concentrated to dryness under reduced pressure to give crude E1 (950 mg, 88.05% yield) as light-yellow oil which was used at the next step directly without further purification. LC/MS (ESI) m/z: 206 (M+H)⁺.

Synthesis of E2: To a mixture of E1 (950 mg, 4.63 mmol) and tert-butyl prop-2-yn-1-ylcarbamate (719 mg, 4.63 mmol) in MeOH (40 mL) and H₂O (10 mL) was added [bis(trifluoroacetoxy)iodo]benzene (2.59 g, 6.02 mmol) at 0° C. under N₂ atmosphere. The resulting mixture was stirred at room temperature for 16 hrs. Then the mixture was diluted with water (60 mL) and extracted with EtOAc (40 mL) twice. The combined organic layers were separated, washed with brine (50 mL), dried over anhydrous Na₂SO₄, filtered and concentrated to dryness. The residue was purified via flash column chromatography on silica gel (eluted with PE:EtOAc=100:0 to 2:1) to give E2 (390 mg, 23.50% yield) as white solid. LC/MS (ESI) m/z: 359 (M+H)⁺.

Synthesis of E3: To a mixture of E2 (390 mg, 1.09 mmol) in DCM (4 mL) was added TFA (1 mL) at 0° C. dropwise under N₂ atmosphere. The resulting mixture was stirred at room temperature for 1 hr. Then the mixture was concentrated to dryness under reduced pressure to give crude E3 (281 mg, 99.98% yield) as yellow oil which was used at the next step directly without further purification. LC/MS (ESI) m/z: 259 (M+H)⁺.

Synthesis of E4: To a mixture of E3 (53 mg, 0.21 mmol) and TEA (64 mg, 0.63 mmol) in THF (5 mL) was added CDI (36 mg, 0.22 mmol) at 0° C., the resulting mixture was stirred at room temperature for 45 mins. Then the mixture was concentrated under reduced pressure to give crude E4 (72 mg, 99.57% yield) as yellow oil which was used at the next step directly without further purification. LC/MS (ESI) m/z: 353 (M+H)⁺.

Synthesis of E5-1 and E5-2: To a solution of C13 (42 mg, 0.16 mmol) in THF (5 mL) were added TEA (162 mg, 1.60 mmol) and E4 (72 mg, 0.20 mmol) at 0° C., the resulting mixture was stirred at 60° C. for 16 hrs. Then the mixture was diluted with H₂O (30 mL) and extracted with EtOAc (30 mL×2). The combined organic layers were separated, washed with brine (30 mL), dried over anhydrous Na₂SO₄, filtered and concentrated to dryness under reduced pressure. The residue was purified by column chromatography on silica gel (eluted with DCM:MeOH=100:0 to 10:1) to give E5 (31.2 mg, 35.93% yield) as white solid. LC/MS (ESI) m/z: 551 (M+H)⁺. ¹H NMR (400 MHz, MeOD) δ 7.85 (d, J=7.8 Hz, 1H), 7.77 (s, 1H), 7.61 (t, J=8.0 Hz, 1H), 7.42 (d, J=8.3 Hz, 1H), 6.74 (s, 1H), 4.61-4.52 (m, 2H), 4.12-4.01 (m, 1H), 4.00-3.90 (m, 1H), 3.83-3.71 (m, 1H), 3.32-3.31 (m, 0.5H), 3.25-3.19 (m, 1H), 3.19-3.16 (m, 1H), 3.16-3.10 (m, 0.5H), 2.72 (d, J=1.1 Hz, 3H), 2.68-2.59 (m, 1H), 2.59-2.52 (m, 1H), 2.38-2.32 (m, 0.5H), 2.31-2.22 (m, 2H), 2.18-2.10 (m, 0.5H), 2.01-1.90 (m, 1H), 1.04-0.96 (m, 2H), 0.87-0.79 (m, 2H). ¹⁹F NMR (376 MHz, MeOD) δ −59.47 (s). This product was further purified via SFC (ChiralPak IB 250×30 mm I.D., 5 μm; mobile phase: A for CO₂ and B for MeOH+0.1% NH₃H₂O; B %: 35%-35%, 3.0 min; 120 min) to give E5-1 (9.5 mg, 10.94% yield) as white solid. ¹H NMR (400 MHz, MeOD) δ 7.86-7.80 (m, 1H), 7.75 (s, 1H), 7.59 (t, J=8.0 Hz, 1H), 7.39 (d, J=8.5 Hz, 1H), 6.72 (s, 1H), 4.59-4.50 (m, 2H), 4.12-4.02 (m, 1H), 3.96-3.88 (m, 1H), 3.81-3.69 (m, 1H), 3.29-3.27 (m, 1H), 3.23-3.16 (m, 1H), 3.15-3.08 (m, 1H), 2.69 (s, 3H), 2.63-2.56 (m, 1H), 2.56-2.49 (m, 1H), 2.29-2.21 (m, 2H), 2.15-2.08 (m, 1H), 2.01-1.92 (m, 1H), 1.02-0.94 (m, 2H), 0.85-0.77 (m, 2H). ¹⁹F NMR (376 MHz, MeOD) δ −59.48 (s); and E5-2 (8.5 mg, 9.79% yield) as white solid. ¹H NMR (400 MHz, MeOD) δ 7.83 (d, J=7.8 Hz, 1H), 7.59 (t, J=8.0 Hz, 1H), 7.39 (d, J=8.6 Hz, 1H), 6.72 (s, 1H), 4.58-4.48 (m, 2H), 4.05-3.98 (m, 1H), 3.97-3.89 (m, 1H), 3.80-3.68 (m, 1H), 3.21-3.16 (m, 1H), 3.16-3.12 (m, 2H), 2.69 (s, 3H), 2.65-2.59 (m, 1H), 2.59-2.52 (m, 1H), 2.36-2.30 (m, 1H), 2.28-2.19 (m, 2H), 1.94-1.86 (m, 1H), 1.01-0.95 (m, 2H), 0.84-0.77 (m, 2H). ¹⁹F NMR (376 MHz, MeOD) δ −59.48 (s).

### Procedure 6: Synthesis of (9R)-9-(3-(4-chloro-2-fluoro-5-methylbenzyl)-1-cyclopropylureido)-N-methyl-3-oxo-2,7-diazaspiro[4.5]decane-7-carboxamide (F5)

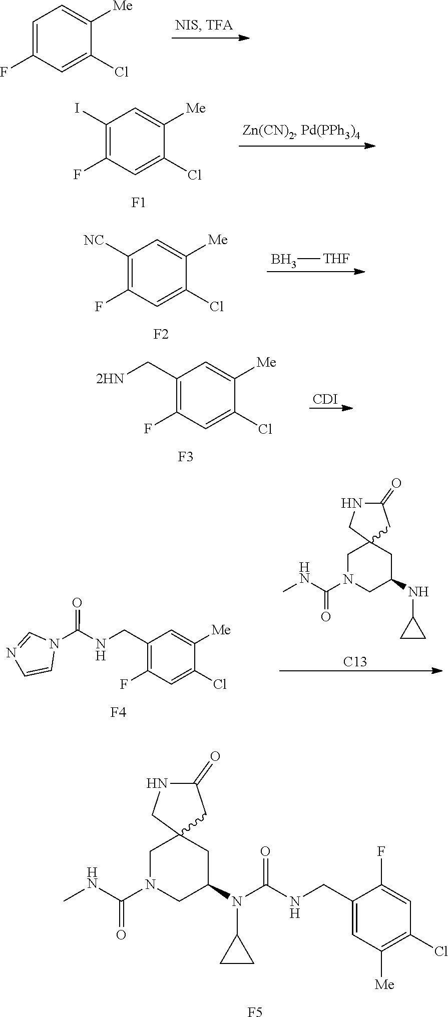

Synthesis of F1: To a solution of 2-chloro-4-fluoro-1-methylbenzene (17.0 g, 117.6 mmol) in TFA (170 mL) was added NIS (29.1 g, 129.3 mmol) at 0° C. in portions. The resulting mixture was stirred at room temperature for 16 hours. Then the mixture was concentrated under reduced pressure, the residue was diluted with DCM (200 mL) and washed with aq. NaHCO₃ (150 ml*3), the organic layer was separated, washed with brine (100 mL), dried over anhydrous Na₂SO₄, filtered and concentrated under reduced pressure to give crude F1 (27.7 g, 87.1% yield) as a white solid which was used at the next step directly without further purification. ¹H NMR (400 MHz, MeOD-d4) δ 7.71 (dd, J=6.8, 0.5 Hz, 1H), 7.17 (d, J=7.8 Hz, 1H), 2.30 (s, 3H).

Synthesis of F2: To a solution of F1 (27 g, 99.8 mmol) in DMF (270 mL) was added Zn(CN)₂ (12.9 g, 109.8 mmol), followed by the addition of Pd(PPh₃)₄ (5.7 g, 4.9 mmol). The resulting mixture was stirred at 100° C. for 16 hours under N₂ atmosphere. After cooling to room temperature, the mixture was diluted with EtOAc (400 mL) and filtered. The filtrate was washed with saturated aq. NH₄Cl (200 mL) twice. The organic layer was separated, washed with brine (200 mL), dried over anhydrous Na₂SO₄ and concentrated to dryness under reduced pressure. The residue was purified by flash column chromatography on silica gel (eluted with PE:EtOAc=100:0 to 95:5) to give F2 (10.0 g, 59.1% yield) as white solid.

Synthesis of compound F3: To a solution of F2 (10 g, 58.9 mmol) in anhydrous THF (100 mL) was added BH₃-THF (295 mL, 1M in THF) drop-wise at 0° C. The resulting mixture was stirred at room temperature for 16 hours under N₂ atmosphere. LCMS showed the starting material was consumed completely. Then the mixture was quenched with MeOH (80 mL) dropwise and then concentrated to dryness under reduced pressure. The residue was diluted with EtOAc (150 mL) and washed with aq. HCl (100 mL, 1 N). The aqueous phase was separated, basified with 15% aq. NaOH to pH=10. Then the mixture was extracted with EtOAc (100 mL*3), the combined organic layers were separated, washed with brine (100 mL), dried over Na₂SO₄, filtered and concentrated under reduced pressure to give crude 4 (5.5 g, 53.4% yield) as colorless oil which was used directly at the next step without further purification. LCMS: ESI m/z: 174 (M+H)⁺.

Synthesis of F4: To a solution of F3 (36 mg, 0.21 mmol) in THF (3 mL) was added CDI (36 mg, 0.22 mmol) at 0° C. The resulting mixture was stirred at room temperature for 50 mins. Then the mixture was concentrated under reduced pressure to give crude F4 (55 mg, 98.99% yield) as colorless oil which was used at the next step directly without further purification. LC/MS (ESI) m/z: 268 (M+H)⁺.

Synthesis of F5: To a solution of C13 (42 mg, 0.16 mmol) in THF (5 mL) were added TEA (162 mg, 1.60 mmol) and F4 (55 mg, 0.21 mmol), the resulting mixture was stirred at 60° C. for 16 hrs. The mixture was diluted with H₂O (30 mL) and extracted with EtOAc (20 mL×2). The combined organic layers were separated, washed with brine (20 mL), dried over anhydrous Na₂SO₄, filtered and concentrated to dryness under reduced pressure. The residue was purified by prep-HPLC to give F5 (20.7 mg, 28.19% yield) as white solid. LC/MS (ESI) m/z: 466 (M+H)⁺. ¹H NMR (400 MHz, MeOD) δ 7.26 (d, J=8.0 Hz, 1H), 7.15 (d, J=9.8 Hz, 1H), 6.98-6.88 (m, 1H), 4.46-4.33 (m, 2H), 4.12-4.01 (m, 1H), 3.96-3.86 (m, 1H), 3.82-3.68 (m, 1H), 3.32-3.30 (m, 0.5H), 3.24-3.18 (m, 1H), 3.18-3.15 (m, 1H), 3.15-3.09 (m, 0.5H), 2.72 (d, J=0.9 Hz, 3H), 2.66-2.57 (m, 1H), 2.57-2.48 (m, 1H), 2.39-2.34 (m, 0.5H), 2.33 (s, 3H), 2.31-2.21 (m, 2H), 2.17-2.10 (m, 0.5H), 2.00-1.86 (m, 1H), 1.01-0.91 (m, 2H), 0.82-0.72 (m, 2H). ¹⁹F NMR (376 MHz, MeOD) δ −123.07 (d, J=4.4 Hz).

### Procedure 7: Synthesis of 1-((9R)-7-acetyl-3-oxo-2,7-diazaspiro[4.5]decan-9-yl)-1-cyclopropyl-3-(2-fluoro-4-(trifluoromethoxy)benzyl)urea (G3)

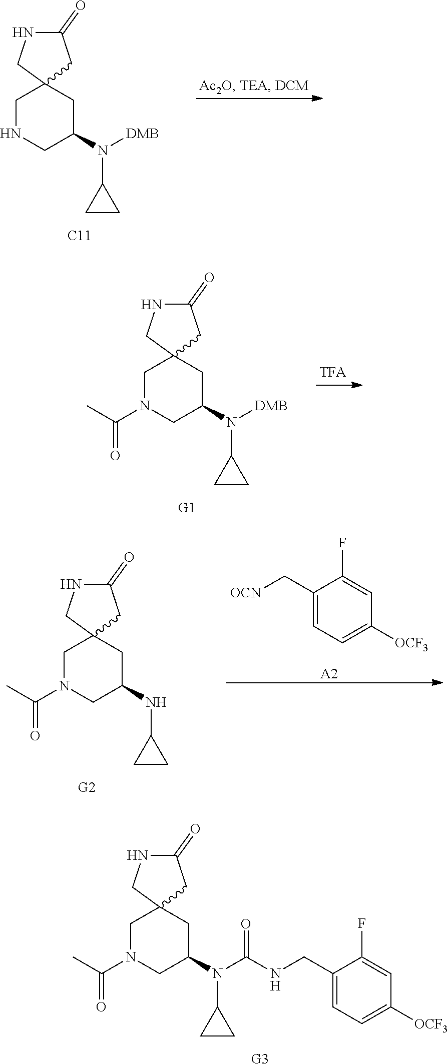

### Step 1: Synthesis of G1

To a mixture of C11 (42 mg, 0.12 mmol) and TEA (36 mg, 0.36 mmol) in DCM (6 mL) was added Ac₂O (26 mg, 0.18 mmol) dropwise at 0° C. Then the resulting mixture was stirred at room temperature for 2 hrs as monitored by TLC. Then the mixture was diluted with water (20 mL) and extracted with EtOAc (20 mL) twice. The combined organic layers were separated, dried over anhydrous Na₂SO₄, filtered and concentrated to dryness. The residue was purified via flash column chromatography on silica gel (eluted with PE/EtOAc=100:0 to 3:1) to give G1 (32 mg, 66.7% yield) as colorless oil. LC/MS (ESI) m/z: 402 (M+H)⁺.

Synthesis of G2: A solution of G1 (32 mg, 0.08 mmol) in TFA (5 mL) was stirred at 80° C. for 2 hrs. Then the mixture was concentrated to dryness under reduced pressure. The residue was diluted with diluted with EtOAc (15 mL) and washed with saturated NaHCO₃ solution (20 mL). The organic layer was separated, washed with brine (20 mL), dried over anhydrous Na₂SO₄, filtered and concentrated to give crude G2 (18 mg, 90.2% yield) as yellow oil which was used at the next step directly without further purification. LC/MS (ESI) m/z: 252 (M+H)⁺.

Synthesis of G3: To a mixture of G2 (18 mg, 0.07 mmol) in THF (5 mL) was added TEA (22 mg, 0.21 mmol) and A2 (32 mg, 0.11 mmol). The resulting mixture was stirred at 60° C. for 16 hrs. Then the mixture was diluted with water (20 mL) and extracted with EtOAc (20 mL) twice. The combined organic layers were washed with brine (20 mL), dried over anhydrous Na₂SO₄, filtered and concentrated to dryness. The residue was purified via prep-HPLC to give G3 (8 mg, 23.5% yield) as white solid. LC/MS (ESI) m/z: 487 (M+H)⁺. ¹H NMR (400 MHz, MeOD) δ 7.49-7.37 (m, 1H), 7.09 (t, J=7.0 Hz, 2H), 4.53 (t, J=11.8 Hz, 1H), 4.43 (t, J=10.7 Hz, 2H), 3.90-3.77 (m, 2H), 3.71-3.47 (m, 1H), 3.43-3.34 (m, 1H), 3.28-3.21 (m, 1H), 3.19 (s, 2H), 3.17-2.93 (m, 1H), 2.57-2.45 (m, 2H), 2.39-2.22 (m, 2H), 2.20 (d, J=17.4 Hz, 1H), 2.13 (d, J=3.2 Hz, 2H), 2.09 (d, J=2.3 Hz, 1H), 2.04-1.84 (m, 1H), 1.02-0.92 (m, 2H), 0.85-0.69 (m, 2H); ¹⁹F NMR (376 MHz, MeOH-d₄) δ −59.78 (s), −116.96 (s).

### Procedure 8: Synthesis of 1-((9R)-7-acetyl-3-oxo-2,7-diazaspiro[4.5]decan-9-yl)-1-cyclopropyl-3-(2-fluoro-4-(trifluoromethoxy)benzyl)urea (H8)

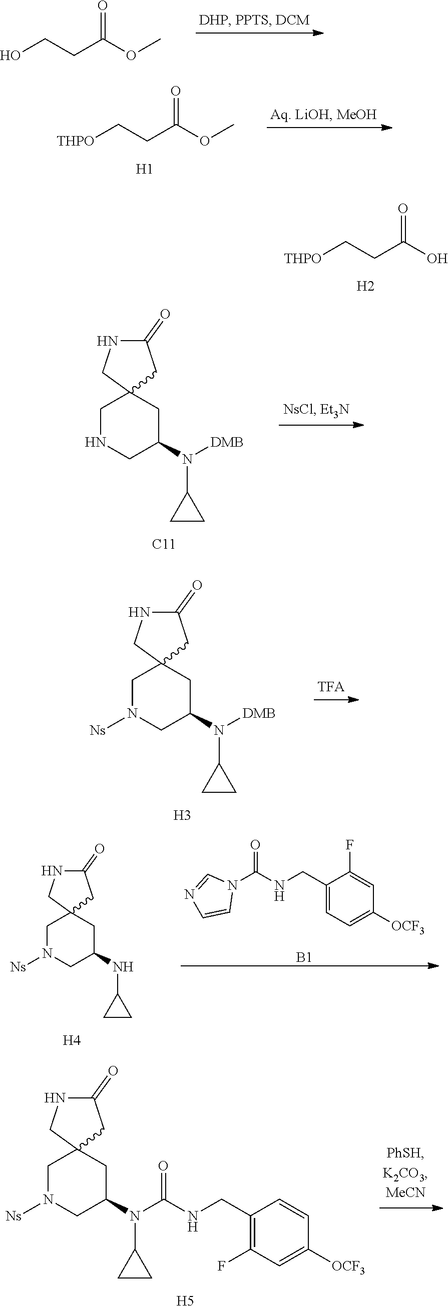

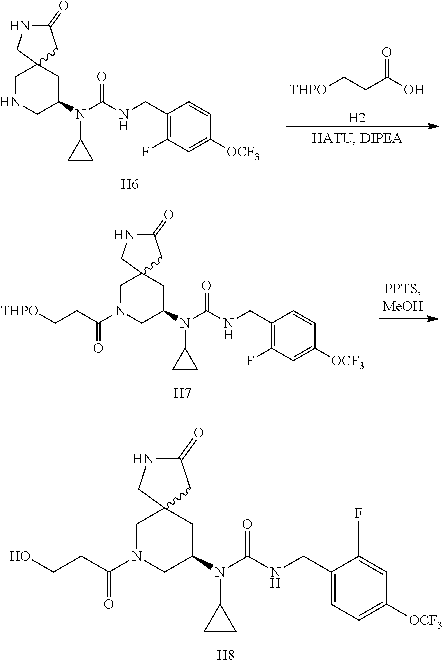

Synthesis of H1: To a solution of methyl 3-hydroxypropanoate (1 g, 9.61 mmol) in DCM (20 mL) was added DHP (1.13 g, 13.45 mmol) and PPTS (121 mg, 0.48 mmol) at room temperature. The resulting mixture was stirred at room temperature for 2 hrs. Then the mixture was concentrated under reduced pressure to dryness. The residue was diluted with EtOAc (30 mL) and washed with water (30 mL) and brine (30 mL). The organic layer was separated, dried over anhydrous Na₂SO₄, filtered and concentrated under reduced pressure to afford crude H1 (1.78 g, 98.5% yield) as colorless oil which was used at the next step directly without further purification.

Synthesis of H2: To a solution of H1 (1.78 g, 9.47 mmol) in MeOH (12 mL) and H₂O (12 mL) was added LiOH (453 mg, 18.91 mmol) in portions. The resulting mixture was stirred at room temperature for 2 hrs. Then the mixture was adjusted to pH=5 with aq. HCl (1 M) and extracted with EtOAc (30 mL) twice. The combined organic layers were separated, washed with water (30 mL) and brine (30 mL), dried over anhydrous Na₂SO₄, filtered and concentrated under reduced pressure to afford crude H2 (1.59 g, 96.5% yield) as colorless oil which was used in the next step directly without further purification. LC/MS (ESI) m/z: 173 (M−H)⁻.

Synthesis of H3: To a solution of C11 (102 mg, 0.28 mmol) in DCM (6 mL) were added TEA (85 mg, 0.84 mmol) and NsCl (82 mg, 0.36 mmol) drop-wise at 0° C. and the resulting mixture was stirred at room temperature for 18 hrs. Then the mixture was diluted with water (20 mL) and extracted with DCM (15 mL) twice. The combined organic layers were separated, washed with brine (20 mL), dried over anhydrous Na₂SO₄, filtered and the filtrate was concentrated to dryness in vacuo. The residue was purified via flash column chromatography on silica gel (eluted with PE/EtOAc=100:0 to 2:1) to give H3 (116 mg, 75% yield) as yellow solid. LC/MS (ESI) m/z: 545 (M+H)⁺.

Synthesis of H4: A mixture of H3 (116 mg, 0.21 mmol) in TFA (4 mL) was stirred at 80° C. for 3 hrs. Then the mixture was concentrated under reduced pressure to give crude H4 (76 mg, 90.5% yield) as purple oil which was used at the next step directly without further purification. LC/MS (ESI) m/z: 395 (M+H)⁺.

Synthesis of H5: To a solution of H4 (76 mg, 0.19 mmol) in MeCN (6 mL) were added TEA (0.08 mL, 0.57 mmol) and B1 (64 mg, 0.21 mmol) at 0° C. and the resulting mixture was stirred at 80° C. for 18 hrs. Then the mixture was diluted with water (20 mL) and extracted with DCM (20 mL) twice. The combined organic layers were washed with brine (20 mL), dried over anhydrous Na₂SO₄, filtered and the filtrate was concentrated to dryness in vacuo. The residue was purified via flash column chromatography on silica gel (eluted with DCM/MeOH=100:0 to 12:1) to give H5 (74 mg, 61% yield) as yellow solid. LC/MS (ESI) m/z: 630 (M+H)⁺.

Synthesis of H6: To a mixture of H5 (74 mg, 0.12 mmol) and K₂CO₃ (162 mg, 1.2 mmol) in MeCN (8 mL) was added PhSH (73 mg, 0.6 mmol) drop-wise at 0° C. and the resulting mixture was stirred at 70° C. for 18 hrs. Then the mixture was diluted with EtOAc (20 mL), filtered and the filtrate was concentrated to dryness in vacuo. The residue was purified via flash column chromatography on silica gel (eluted with DCM/MeOH=100:0 to 10:1) to give H6 (50 mg, 95.1% yield) as yellow oil. LC/MS (ESI) m/z: 445 (M+H)⁺.

Synthesis of H7: To a mixture of H6 (50 mg, 0.12 mmol) and DIEA (0.1 mL, 0.6 mmol) in DMF (5 mL) was added H2 (31 mg, 0.18 mmol) and HATU (67 mg, 0.18 mmol) at 0° C. and the resulting mixture was stirred at room temperature for 2 hrs. Then the mixture was diluted with water (20 mL) and extracted with EtOAc (20 mL) twice. The combined organic layers were washed with brine (20 mL), dried over anhydrous Na₂SO₄, filtered and the filtrate was concentrated to give crude H7 (70 mg, 99.0% yield) as yellow solid which was used at the next step directly without further purification. LC/MS (ESI) m/z: 601 (M+H)⁺.

Synthesis of H8: To a solution of H7 (70 mg, 0.12 mmol) in MeOH (5 mL) was added PPTS (90 mg, 0.36 mmol) at 0° C. and the resulting mixture was stirred at room temperature for 18 hrs. Then the mixture was diluted with water (20 mL) and extracted with DCM (20 mL) twice. The combined organic layers were washed with brine (20 mL), dried over anhydrous Na₂SO₄, filtered and the filtrate was concentrated to dryness in vacuo. The residue was purified via prep-HPLC to give H8 (17 mg, 27.4% yield) as white solid. LC/MS (ESI) m/z: 517 (M+H)⁺. ¹H NMR (400 MHz, MeOH-d₄) δ 7.48-7.39 (m, 1H), 7.09 (t, J=7.6 Hz, 2H), 7.03-6.92 (m, 1H), 4.57 (t, J=11.6 Hz, 1H), 4.50-4.37 (m, 2H), 3.99 (d, J=8.2 Hz, 1H), 3.93-3.72 (m, 3H), 3.61 (d, J=12.6 Hz, 1H), 3.49-3.32 (m, 1H), 3.25-3.12 (m, 2H), 3.08-2.94 (m, 1H), 2.74 (dt, J=14.9, 6.1 Hz, 1H), 2.66-2.43 (m, 3H), 2.37-2.10 (m, 3H), 2.03-1.84 (m, 1H), 0.96 (d, J=6.2 Hz, 2H), 0.78 (d, J=12.1 Hz, 2H); ¹⁹F NMR (376 MHz, MeOH-d₄) δ −59.78 (s), −116.98 (s).

### Procedure 9: Synthesis of (9R)-9-(1-cyclopropyl-3-(2-fluoro-4-(trifluoromethoxy)benzyl)ureido)-N-methyl-2-oxo-1,3,7-triazaspiro[4.5]decane-7-carboxamide (710)

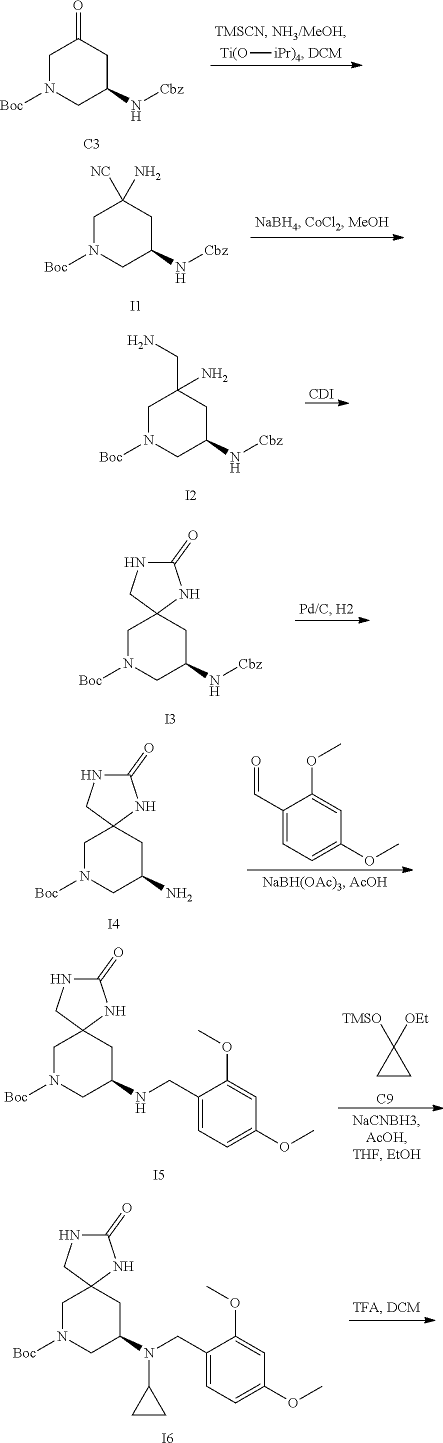

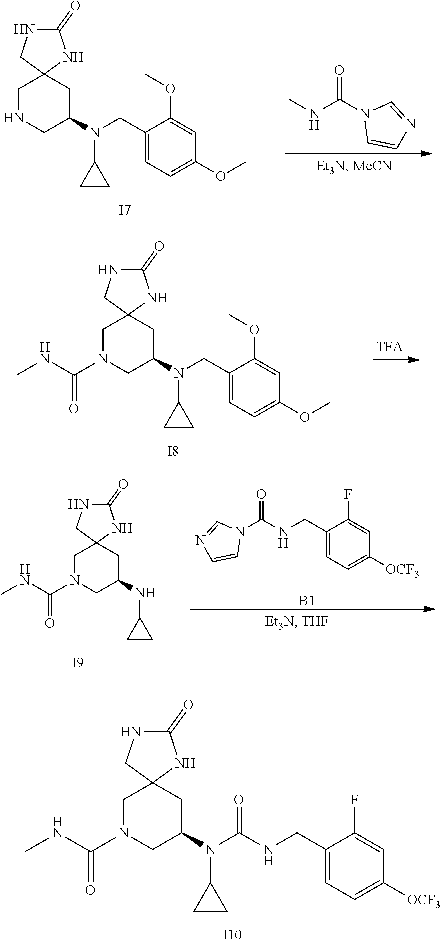

Synthesis of I1: To a mixture of C3 (470 mg, 1.35 mmol) and Ti(Oi-Pr)₄ (422 mg, 1.49 mmol) in DCM (10 mL) was added NH₃/MeOH (7N, 1.9 mL) at 0° C., and the mixture was stirred at room temperature for 2 hrs. Then TMSCN (161 mg, 1.62 mmol) was added into the above mixture at 0° C., and the resulting mixture was stirred at room temperature for another 16 hrs in a sealed tube. Then the mixture was diluted with H₂O (30 mL), filtered and the filtrate was extracted with DCM (20 mL×2). The combined organic layers were separated, washed with brine (30 mL), dried over anhydrous Na₂SO₄, filtered and concentrated to dryness. The residue was purified by column chromatography on silica gel (eluted with DCM:EtOAc=100:0 to 25:1) to give 11 (428 mg, 84.73% yield) as colorless oil. LC/MS (ESI) m/z: 375 (M+H)⁺.

Synthesis of I2: To a solution of I1 (428 mg, 1.14 mmol) in MeOH (10 mL) were added CoCl₂ (14 mg, 0.11 mmol) and NaBH₄ (87 mg, 2.28 mmol) at 0° C., the resulting mixture was stirred at 0° C. for 1 hr. Then the mixture was quenched with aq. NaOH (30 mL, 1 N) and extracted with DCM (20 mL×2). The combined organic layers were washed with brine (30 mL), dried over anhydrous Na₂SO₄, filtered and concentrated to give crude 12 (401 mg, 92.47% yield) as colorless oil which was used at the next step directly without further purification. LC/MS (ESI) m/z: 379 (M+H)⁺.

Synthesis of 13: To a solution of I2 (401 mg, 1.06 mmol) in THF (10 mL) was added CDI (76 mg, 0.74 mmol) at 0° C., the resulting mixture was stirred at 0° C. for 30 mins. LCMS indicated the complete consumption of the starting material. Then the mixture was stirred at 50° C. for another 2 hrs. After cooling to room temperature, the mixture was concentrated to dryness. The residue was purified by column chromatography on silica gel (eluted with DCM:EtOAc=100:0 to 16:1) to give 13 (305 mg, 71.34% yield) as colorless oil. LC/MS (ESI) m/z: 405 (M+H)⁺.

Synthesis of I4: To a solution of 4 (305 mg, 0.75 mmol) in MeOH (10 mL) was added Pd/C (300 mg, 10% w/w), the resulting mixture was degassed under N₂ atmosphere for three times and stirred at room temperature for 2 hrs under H2 atmosphere. Then the mixture was filtered and the filtrate was concentrated to give crude 14 (193 mg, 94.69% yield) as colorless oil which was used at the next step directly without further purification. LC/MS (ESI) m/z: 271 (M+H)⁺.

Synthesis of I5: To a mixture of 14 (193 mg, 0.71 mmol) and 2,4-dimethoxybenzaldehyde (118 mg, 0.71 mmol) in DCM (8 mL) was added AcOH (85 mg, 1.42 mmol) and the mixture was stirred at room temperature for 1 hr. Then NaBH(OAc)₃ (452 mg, 2.13 mmol) was added into the above mixture at 0° C., the resulting mixture was stirred at room temperature for 16 hrs. Then the mixture was concentrated to dryness. The residue was purified by column chromatography on silica gel (eluted with DCM:MeOH=100:0 to 10:1) to give 15 (288 mg, 95.83% yield) as colorless oil. LC/MS (ESI) m/z: 421 (M+H)⁺.

Synthesis of I6: To a mixture of I5 (288 mg, 0.69 mmol) and AcOH (414 mg, 6.90 mmol) in EtOH (5 mL) and THF (10 mL) was added C9 (300 mg, 1.73 mmol) and NaBH₃CN (152 mg, 2.42 mmol). The resulting mixture was stirred at 80° C. for 3 hrs. Then the mixture was concentrated to dryness, the residue was diluted with EtOAc (30 mL) and washed with saturated NaHCO₃ solution (30 mL). The organic layer was separated, washed with brine (30 mL), dried over anhydrous Na₂SO₄, filtered and concentrated to dryness. The residue was purified by column chromatography on silica gel (eluted with DCM:MeOH=100:0 to 25:1) to give 16 (235 mg, 74.50% yield) as colorless oil. LC/MS (ESI) m/z: 461 (M+H)⁺.

Synthesis of I7: To a solution of I6 (235 mg, 0.51 mmol) in DCM (8 mL) was added TFA (2 mL) at 0° C. and the resulting mixture was stirred at room temperature for 1 hr. LCMS indicated the complete consumption of the starting material. Then the mixture was concentrated to give crude I7 (179 mg, 97.33% yield) as colorless oil. LC/MS (ESI) m/z: 361 (M+H)⁺.

Synthesis of I8: To a mixture of I7 (179 mg, 0.50 mmol) and DIEA (323 mg, 2.5 mmol) in MeCN (10 mL) was added N-methyl-1H-imidazole-1-carboxamide (313 mg, 2.5 mmol) and the resulting mixture was stirred at 60° C. for 16 hrs. Then the mixture was diluted with H₂O (30 mL) and extracted with EtOAc (20 mL×2), the combined organic layers were washed with brine (30 mL), dried over anhydrous Na₂SO₄, filtered and concentrated to dryness. The residue was purified by column chromatography on silica gel (eluted with DCM:MeOH=100:0 to 9:1) to give 18 (110 mg, 53.05% yield) as colorless oil. LC/MS (ESI) m/z: 418 (M+H)⁺.

Synthesis of I9: A round-bottom flask was charged with I8 (110 mg, 0.26 mmol) and TFA (5 mL), the resulting mixture was stirred at 80° C. for 4 hrs. LCMS indicated the complete consumption of the starting material. Then the mixture was concentrated to give crude I9 (58 mg, 82.35% yield) as a purple oil which was used at the next step directly without further purification. LC/MS (ESI) m/z: 268 (M+H)⁺.

Synthesis of I10: To a mixture of I9 (58 mg, 0.22 mmol) and TEA (111 mg, 1.10 mmol) in THF (8 mL) was added B1 (67 mg, 0.22 mmol) at 0° C., the resulting mixture was stirred at 65° C. for 6 hrs. The mixture was diluted with H₂O (30 mL) and extracted with EtOAc (20 mL×2). The combined organic layers were washed with brine (30 mL), dried over anhydrous Na₂SO₄, filtered and concentrated to dryness. The residue was purified by prep-HPLC to give 110 (41.9 mg, 38.42% yield) as white solid. LC/MS (ESI) m/z: 503 (M+H)⁺. ¹H NMR (400 MHz, MeOD) δ 7.43 (t, J=8.6 Hz, 1H), 7.09 (t, J=7.8 Hz, 2H), 7.04-6.94 (m, 1H), 4.51-4.37 (m, 2H), 4.14-4.00 (m, 1H), 4.00-3.93 (m, 0.5H), 3.91-3.84 (m, 0.5H), 3.83-3.75 (m, 0.5H), 3.72-3.61 (m, 0.5H), 3.40-3.34 (m, 0.5H), 3.29-3.24 (m, 1H), 3.23-3.16 (m, 1H), 3.12-3.05 (m, 0.5H), 2.73-2.69 (m, 3H), 2.67 (s, 0.5H), 2.62-2.56 (m, 1H), 2.56-2.50 (m, 0.5H), 2.43-2.29 (m, 1H), 2.04-1.88 (m, 1H), 1.00-0.89 (m, 2H), 0.83-0.68 (m, 2H). ¹⁹F NMR (377 MHz, MeOD) δ −59.77 (s), −116.97 (s).

### Procedure 10: Synthesis of 9-(1-cyclopropyl-3-(2-fluoro-4-(trifluoromethoxy) benzyl)ureido)-N-methyl-2-oxa-7-azaspiro[4.5]decane-7-carboxamide (J15)

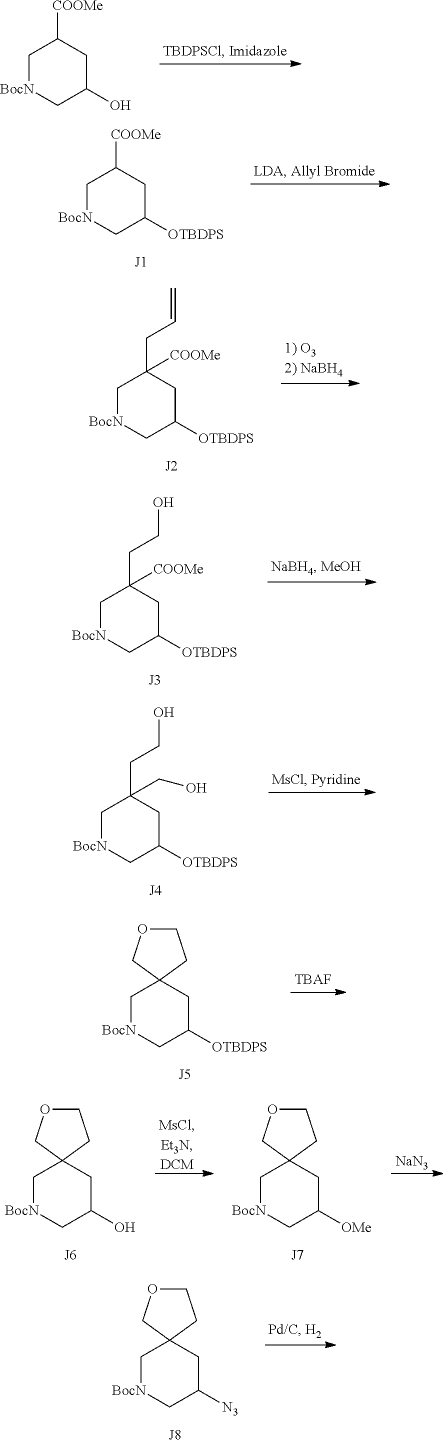

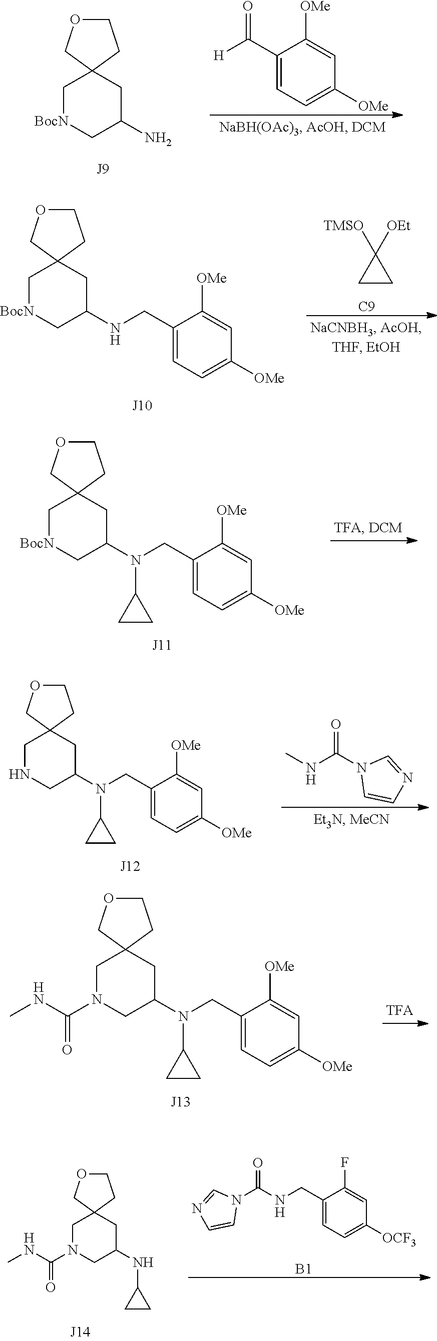

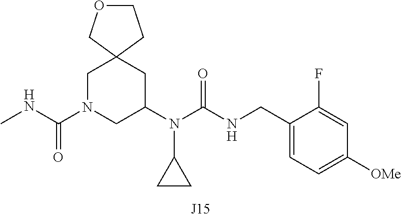 [SMILES: CNC(=O)N1CC(N(C(=O)NCc2ccc(OC)cc2F)C2CC2)CC2(CCOC2)C1]

Synthesis of J1: To a mixture of 1-(tert-butyl) 3-methyl 5-hydroxypiperidine-1,3-dicarboxylate (2 g, 7.713 mmol) and imidazole (1.49 g, 21.951 mmol) in DMF (25 mL) was added TBDPSCl (3.02 g, 10.976 mmol) dropwise at 0° C. under N₂ atmosphere. The resulting mixture was stirred at room temperature for 16 hours under N₂ atmosphere. Then the mixture was diluted with EtOAc (80 mL) and washed with saturated NH₄Cl solution (80 mL) twice and brine (100 mL). The organic layer was separated, dried over anhydrous Na₂SO₄ and filtered. The filtrate was evaporated to dryness. The residue was purified by column chromatography on silica gel (eluted with PE:EtOAc=100:0 to 10:1) to give J1 (3.56 g, 95.1% yield) as colorless oil. LC/MS (ESI) m/z: 498 (M+H)⁺.

Synthesis of J2: To a solution of J1 (2.5 g, 4.885 mmol) in anhydrous THF (60 mL) was added LDA (6.1 mL, 2M in THF) dropwise at −78° C. under N₂ atmosphere. The resulting mixture was stirred at −78° C. for 1.5 hrs under N₂ atmosphere. Then 3-bromoprop-1-ene (0.64 mL, 7.328 mmol) was added into the above mixture dropwise. The resulting mixture was stirred at −78° C. for another 2 hrs under N₂ atmosphere. Then the mixture was quenched with saturated NH₄Cl solution (60 mL) and extracted with EtOAc (60 mL) twice. The combined organic layer was separated, washed with brine (80 mL), dried over anhydrous Na₂SO₄ and filtered. The filtrate was evaporated to dryness. The residue was purified by column chromatography on silica gel (eluted with PE:EtOAc=100:0 to 20:1) to give J2 (2.2 g, 81.6% yield) as colorless oil. LC/MS (ESI) m/z: 538 (M+H)⁺.

Synthesis of J3: To a mixture of J2 (2.2 g, 4.091 mmol) in DCM (30 mL) and MeOH (20 mL) was bubbled with 02 for 3 minutes and then 03 for 1 hour at −78° C. under N₂ atmosphere. Then the mixture was purged with 02 for 3 minutes and then N₂ for 3 minutes before NaBH₄ (0.40 g, 11.961 mmol) was added into the above mixture. The resulting mixture was stirred at 0° C. for another 2 hours under N₂ atmosphere. Then the mixture was quenched with saturated NH₄Cl solution (100 mL) and extracted with EtOAc (80 mL) twice. The combined organic layer was separated, washed with brine (100 mL), dried over anhydrous Na₂SO₄ and filtered. The filtrate was evaporated to dryness to give crude J3 (2.0 g, 70% purity, 68.9% yield) as colorless oil which was used at the next step directly without further purification. LC/MS (ESI) m/z: 510 (M+H)⁺.

Synthesis of J4: To a solution of J3 (2 g, 2.747 mmol) in MeOH (20 mL) was added NaBH₄ (930 mg, 27.47 mmol) at 0° C. under N₂ atmosphere. The resulting mixture was stirred at room temperature for 16 hrs under N₂ atmosphere. Then the mixture was quenched with saturated NH₄Cl solution (80 mL) and extracted with DCM (60 mL) twice. The combined organic layers were separated, washed with water (100 mL) and brine (100 mL), dried over anhydrous Na₂SO₄ and filtered. The filtrate was evaporated to dryness. The residue was purified by column chromatography on silica gel (eluted with PE:EtOAc=100:0 to 1:1) to give J4 (1.2 g, 85.0% yield) as colorless oil. LC/MS (ESI) m/z: 514 (M+H)⁺.

Synthesis of J5: To a mixture of J4 (1.2 g, 2.42 mmol) and pyridine (1.11 g, 14.02 mmol) in DCM (25 mL) was added MsCl (803 mg, 7.01 mmol) dropwise at 0° C. The resulting mixture was stirred at room temperature for 22 hours under N₂ atmosphere. Then the mixture was quenched with saturated NaHCO₃ solution (80 mL) and extracted with EtOAc (60 mL) twice. The combined organic layers were separated, washed with water (100 mL) and brine (100 mL), dried over anhydrous Na₂SO₄ and filtered. The filtrate was evaporated to dryness. The residue was purified by column chromatography on silica gel (eluted with PE:EtOAc=100:0 to 8:1) to give J5 (860 mg, 74.27% yield) as colorless oil. LC/MS (ESI) m/z: 496 (M+H)⁺.

Synthesis of J6: To a solution of J5 (860 mg, 1.735 mmol) in THF (20 mL) was added TBAF (3.5 mL, 3.50 mmol, 1M in THF) at 0° C. The resulting mixture was stirred at room temperature for 2 hrs under N₂ atmosphere. Then the mixture was diluted with EtOAc (50 mL) and washed with water (50 mL×4) and brine (50 mL×2). The organic layer was separated, dried over anhydrous Na₂SO₄ and filtered. The filtrate was evaporated to dryness. The residue was purified by column chromatography on silica gel (eluted with PE:EtOAc=100:0 to 1:1) to give J6 (420 mg, 94.1% yield) as colorless oil. LC/MS (ESI) m/z: 258 (M+H)⁺.

Synthesis of J7: To a solution of J6 (280 mg, 1.088 mmol) in DCM (8 mL) was added TEA (330 mg, 3.264 mmol) and MsCl (149 mg, 1.306 mmol) at 0° C. The resulting mixture was stirred at room temperature for 2 hrs under N₂ atmosphere. Then the mixture was diluted with DCM (30 mL) and washed with water (30 mL) and brine (30 mL). The organic layer was separated, dried over anhydrous Na₂SO₄ and filtered. The filtrate was evaporated to dryness to give crude J7 (320 mg, 87.7% yield) as colorless oil which was used at the next step directly without further purification. LC/MS (ESI) m/z: 336 (M+H)⁺.

Synthesis of J8: To a solution of J7 (320 mg, 0.954 mmol) in DMF (8 mL) was added NaN₃ (186 mg, 2.862 mmol) at room temperature under N₂ atmosphere. The resulting mixture was stirred at 90° C. for 16 hours under N₂ atmosphere. Then the mixture was cooled to room temperature and diluted with water (30 mL), extracted with EtOAc (30 mL) twice. The combined organic layers were separated, washed with saturated NH₄Cl solution (30 mL×2) and brine (30 mL×2), dried over anhydrous Na₂SO₄ and filtered. The filtrate was evaporated to dryness to give crude J8 (290 mg, 60% purity, 64.6% yield) as colorless oil which was used at the next step directly without further purification. LC/MS (ESI) m/z: 283 (M+H)⁺.

Synthesis of J9: To a solution of J8 (290 mg, 0.616 mmol) in EtOH (5 mL) was added Pd/C (100 mg, 10% w/w). The resulting mixture was stirred at room temperature for 6 hrs under H2 atmosphere with 25 psi. Then the mixture was diluted with DCM (30 mL) and filtered through a pad of celite. The filtrate was concentrated to dryness. The crude product was purified by column chromatography on silica gel (eluted with DCM:MeOH=100:0 to 10:1) to give J9 (145 mg, 91.8% yield) as colorless oil. LC/MS (ESI) m/z: 257 (M+H)⁺.

Synthesis of J10: To a mixture of J9 (145 mg, 0.566 mmol) and 2,4-dimethoxybenzaldehyde (94 mg, 0.566 mmol) in DCM (8 mL) was added AcOH (102 mg, 1.697 mmol) at 0° C. The resulting mixture was stirred at room temperature for 1 hr. Then NaBH(OAc)₃ (359.74 mg, 1.697 mmol) was added into the above mixture in portions at 0° C. and the resulting mixture was stirred at room temperature for 4 hrs under N₂ atmosphere. The mixture was quenched with saturated NaHCO₃ solution (30 mL) and extracted with DCM (20 mL) twice. The combined organic layers were separated, washed with brine (30 mL), dried over anhydrous Na₂SO₄ and filtered. The filtrate was evaporated to dryness under reduced pressure. The residue was purified by column chromatography on silica gel (eluted with DCM:MeOH=100:0 to 10:1) to give J10 (135 mg, 58.7% yield) as colorless oil. LC/MS (ESI) m/z: 407 (M+H)⁺.

Synthesis of J11: To a mixture of J10 (125 mg, 0.307 mmol) and AcOH (55 mg, 0.922 mmol) in THF (6 mL) and EtOH (3 mL) was added C9 (134 mg, 0.769 mmol) and NaBH₃CN (58 mg, 0.922 mmol). The resulting mixture was stirred at 80° C. for 4 hrs under N₂ atmosphere. After cooling to room temperature, the mixture was diluted with water (20 mL) and basified with saturated NaHCO₃ solution (30 mL) to pH=8. Then the mixture was extracted with EtOAc (20 mL) twice. The combined organic layers were washed with brine (30 mL), dried over anhydrous Na₂SO₄, filtered and concentrated to dryness under reduced pressure. The residue was purified by column chromatography on silica gel (eluted with DCM:MeOH=100:0 to 25:1) to give J11 (95 mg, 67.9% yield) as colorless oil. LC/MS (ESI) m/z: 447 (M+H)⁺.

Synthesis of J12: To a solution of J11 (95 mg, 0.213 mmol) in anhydrous DCM (6 mL) was added TFA (2 mL) at 0° C. dropwise. The resulting mixture was stirred at room temperature for 2 hrs under N₂ atmosphere. Then the reaction mixture was concentrated under reduced pressure to give crude J12 (73 mg, 99.1% yield) as yellow oil which was used at the next step directly without further purification. LC/MS (ESI) m/z: 347 (M+H)⁺.

Synthesis of J13: To a mixture of J12 (73 mg, 0.211 mmol) and TEA (64 mg, 0.632 mmol) in anhydrous MeCN (6 mL) was added N-methyl-1H-imidazole-1-carboxamide (53 mg, 0.421 mmol) at room temperature. The resulting mixture was stirred at 60° C. for 16 hours under N₂ atmosphere. Then the mixture was diluted with water (30 mL) and extracted with EtOAc (20 mL) twice. The combined organic layers were washed with brine (30 mL), dried over anhydrous Na₂SO₄ and filtered. The filtrate was evaporated to dryness under reduced pressure. The residue was purified by column chromatography on silica gel (eluted with DCM:MeOH=100:0 to 20:1) to give 15 (80 mg, 94.1% yield) as colorless oil. LC/MS (ESI) m/z: 404 (M+H)⁺.

Synthesis of J14: A solution of J13 (70 mg, 0.173 mmol) in TFA (5 mL) was stirred at 80° C. for 3 hrs under N₂ atmosphere. After cooling, the mixture was evaporated to dryness under reduced pressure to give crude J14 (42 mg, 95.6% yield) as purple oil which was used at the next step directly without further purification. LC/MS (ESI) m/z: 254 (M+H)⁺.

Synthesis of J15: To a mixture of J14 (42 mg, 0.166 mmol) and TEA (167 mg, 1.66 mmol) in anhydrous THF (4 mL) was added a solution of B1 (75 mg, 0.249 mmol) in anhydrous THF (1 mL) at room temperature. The resulting mixture was stirred at 60° C. for 16 hrs under N₂ atmosphere. Then the mixture was diluted with water (20 mL) and extracted with EtOAc (20 mL) twice. The combined organic layers were separated, washed with brine (20 mL), dried over anhydrous Na₂SO₄ and filtered. The filtrate was evaporated to dryness under reduced pressure. The residue was purified via prep-HPLC to give J15 (33.1 mg, 40.87% yield) as white solid. LC/MS (ESI) m/z: 489 (M+H)⁺. ¹H NMR (400 MHz, CD₃OD-d₄) δ 7.51-7.38 (m, 1H), 7.18-6.98 (m, 2H), 4.52-4.38 (m, 2H), 4.03-3.77 (m, 5H), 3.66-3.54 (m, 1H), 3.48-3.37 (m, 1H), 3.14 (t, J=11.7 Hz, 1H), 2.72 (s, 3H), 2.64-2.50 (m, 2H), 2.36-2.13 (m, 1H), 2.01-1.82 (m, 1H), 1.80-1.63 (m, 2H), 1.04-0.89 (m, 2H), 0.84-0.69 (m, 2H). ¹⁹F NMR (376 MHz, CD₃OD-d₄) δ −59.16-−60.07 (m), −116.66-−117.21 (m).

### Procedure 11: Synthesis of 4-(1-cyclopropyl-3-(2-fluoro-4-(trifluoromethoxy)benzyl)ureido)-N-methyl-9-oxa-2-azaspiro[5.5]undecane-2-carboxamide (K7)

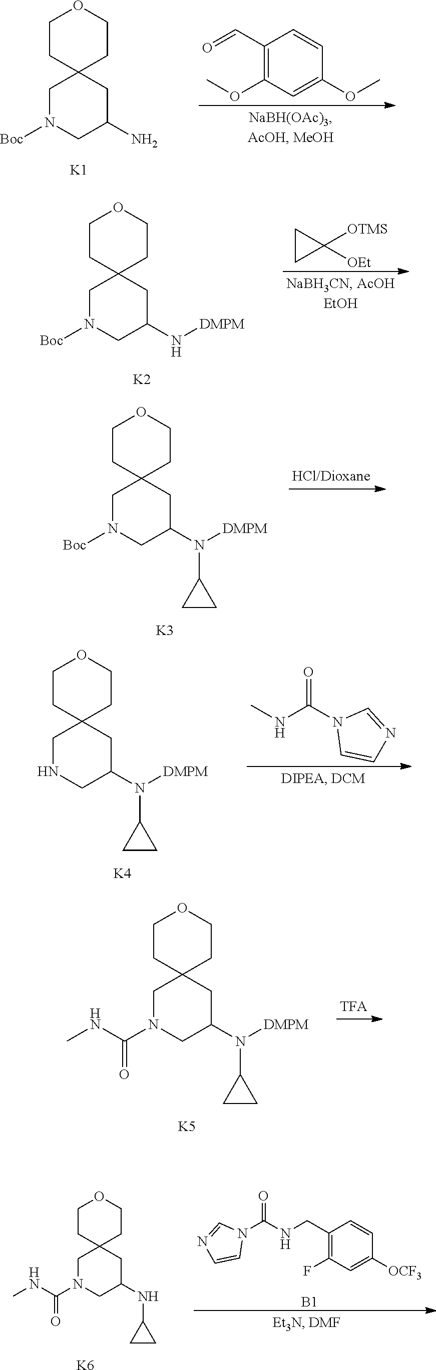

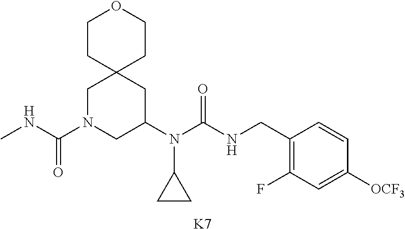

Synthesis of K2: To a mixture of K1 (500 mg, 1.85 mmol) and 2,4-dimethoxybenzaldehyde (307 mg, 1.85 mmol) in MeOH (12 mL) was added AcOH (212 μL, 3.70 mmol) and the mixture was stirred at room temperature for 10 min. Then NaBH(OAc)₃ (784 mg, 3.70 mmol) was added and the resulting mixture was stirred at room temperature for 1 hr. Then the mixture was cooled to 0° C., basified with 5% aqueous ammonia solution, and the residue was extracted with EtOAc (30 mL×3). The organic layers were combined, dried over anhydrous Na₂SO₄, filtered and concentrated to dryness to give crude 2 (728 mg) directly used for the next step without purification. LC/MS (ESI) m/z: 421 (M+H)⁺.

Synthesis of K3: To a mixture of K2 (728 mg, 1.73 mmol) and (1-ethoxycyclopropoxy)trimethylsilane (603 mg, 3.46 mmol) in EtOH (7.5 mL) was added AcOH (595 μL, 10.4 mmol) and NaBH₃CN (218 mg, 3.46 mmol). The resulting mixture was stirred at 80° C. for 18 hrs under N₂ atmosphere. Then the mixture was cooled to 0° C., basified with 5% aqueous ammonia solution, and the residue was extracted with EtOAc (30 mL×3). The organic layers were combined, dried over anhydrous Na₂SO₄, filtered and concentrated to dryness. The residue was purified by column chromatography on silica gel (eluted with Hexanes:EtOAc=100:0 to 0:100) to give K3 (536 mg, 67.2% yield over 2 steps) as colorless oil. LC/MS (ESI) m/z: 461 (M+H)⁺.

Synthesis of K4: To a solution of K3 (500 mg, 1.09 mmol) in DCM (5 mL) was added 1 M HCl in dioxane (1 mL) dropwise at 0° C. and the resulting mixture was stirred at room temperature for 3 hr. LCMS indicated the complete consumption of the starting material. Then the mixture was concentrated to give crude K4 (430 mg, 99.9% yield) as white solid which was used at the next step directly without further purification. LC/MS (ESI) m/z: 361 (M+H)⁺.

Synthesis of K5: To a mixture of K4 (230 mg, 0.58 mmol) and DIPEA (303 μL, 1.74 mmol) in DCM (5 mL) was added N-methyl-1H-imidazole-1-carboxamide (94 mg, 0.75 mmol) and the resulting mixture was stirred at room temp for 16 hrs. Then the mixture was diluted with H₂O (30 mL) and extracted with EtOAc (20 mL×2), the combined organic layers were washed with brine (30 mL), dried over anhydrous Na₂SO₄, filtered and concentrated to give crude K5 (182 mg, 75.4% yield) which was used at the next step directly without further purification. LC/MS (ESI) m/z: 418 (M+H)⁺.

Synthesis of K6: A round-bottom flask was charged with K5 (182 mg, 0.44 mmol) and TFA (4 mL), the mixture was stirred at 80° C. for 1 hr. LCMS indicated the complete consumption of the starting material. Then the mixture was concentrated to give crude K6 as purple oil which was used at the next step directly without further purification. LC/MS (ESI) m/z: 268 (M+H)⁺.

Synthesis of K7: To a mixture of 7 and TEA (303 μL) in DMF (1.5 mL) was added a solution of 8 (1.13 mL, 0.57 mmol), the resulting mixture was stirred at 50° C. for 2 hrs. Then the residue was directly purified by prep HPLC (eluted with H₂O+0.1% TFA:MeCN+0.1% TFA=100:0 to 0:100) to give pure K7 (38 mg, 17% yield over 2 steps) as white solid. LC/MS (ESI) m/z: 503 (M+H)⁺. ¹H NMR (400 MHz, d-DMSO) δ 7.50-7.45 (m, 1H), 7.37-7.35 (m, 1H), 7.28-7.26 (m, 1H), 6.96-6.93 (m, 1H), 6.38-6.37 (m, 1H), 4.36-4.35 (m, 2H), 4.18-4.14 (m, 1H), 3.94-3.91 (m, 1H), 3.78-3.71 (m, 1H), 3.62-3.55 (m, 4H), 3.05-2.99 (m, 1H), 2.59-2.58 (m, 3H), 2.52-2.47 (m, 1H), 2.33-2.30 (m, 1H), 1.86-1.70 (m, 2H), 1.50-1.47 (m, 1H), 1.39-1.30 (m, 3H), 0.94-0.92 (m, 2H), 0.70-0.67 (m, 2H);

### Procedure 12: Synthesis of 4-(1-cyclopropyl-3-(2-fluoro-4-(trifluoromethoxy) benzyl)ureido)-9-oxa-2-azaspiro[5.5]undecane-2-carboxamide (L3)

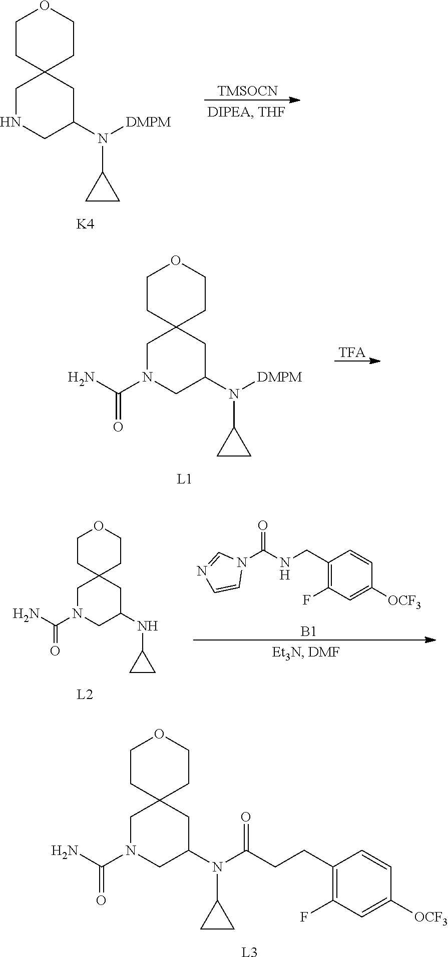

Synthesis of L1: To a mixture of K4 (63 mg, 0.16 mmol) and DIPEA (83 μL, 0.48 mmol) in THF (1.5 mL) was added trimethylsilyl isocyanate (26 μL, 0.19 mmol) and the resulting mixture was stirred at room temp for 16 hrs. Then the mixture was diluted with H₂O (10 mL) and extracted with EtOAc (10 mL×2), the combined organic layers were dried over anhydrous Na₂SO₄, filtered and concentrated to give crude L1 (27 mg, 42% yield) which was used at the next step directly without further purification. LC/MS (ESI) m/z: 404 (M+H)⁺.

Synthesis of 10: A scintillation vial was charged with L1 (27 mg, 68 μmol) and TFA (1 mL), and the mixture was stirred at 80° C. for 1 hr. LCMS indicated the complete consumption of the starting material. Then the mixture was concentrated to give crude L2 as purple oil which was used at the next step directly without further purification. LC/MS (ESI) m/z: 254 (M+H)⁺.

Synthesis of L3: To a mixture of L2 and TEA (47 μL) in DMF (1 mL) was added a solution of B1 (0.18 mL, 88 μmol), and the resulting mixture was stirred at 50° C. for 2 hrs. Then the residue was directly purified by prep HPLC (eluted with H₂O+0.1% TFA:MeCN+0.1% TFA=100:0 to 0:100) to give pure L3 (6.9 mg, 21% yield over 2 steps) as white solid. LC/MS (ESI) m/z: 489 (M+H)⁺. ¹H NMR (400 MHz, d-DMSO) δ 7.48-7.43 (m, 1H), 7.36-7.33 (m, 1H), 7.26-7.24 (m, 1H), 6.93-6.90 (m, 1H), 5.88 (s, 2H), 4.35-4.33 (m, 2H), 4.15-4.12 (m, 1H), 3.95-3.92 (m, 1H), 3.77-3.69 (m, 1H), 3.65-3.51 (m, 4H), 3.05-2.99 (m, 1H), 2.51-2.46 (m, 1H), 2.33-2.30 (m, 1H), 1.84-1.78 (m, 1H), 1.71-1.68 (m, 1H), 1.52-1.48 (m, 1H), 1.38-1.29 (m, 3H), 0.92-0.85 (m, 2H), 0.71-0.64 (m, 2H).

TABLE 1

| Ex- | Pro- | |
|---|---|---|
| ample | cedure | Structure |
| 1A | 3 | 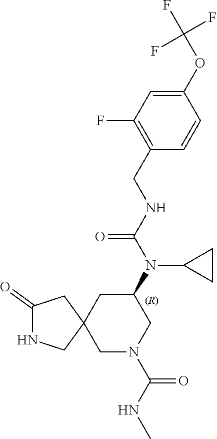 |
| 2A | 9 | 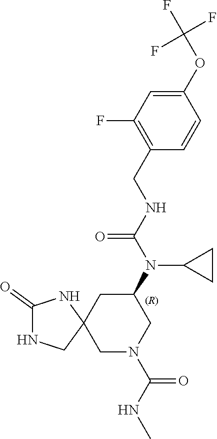 |
| 3A | 10 | 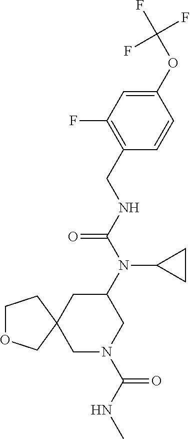 [SMILES: CNC(=O)N1CC(N(C(=O)NCc2ccc(OC(F)(F)F)cc2F)C2CC2)CC2(CCOC2)C1] |
| 4A | 3 | 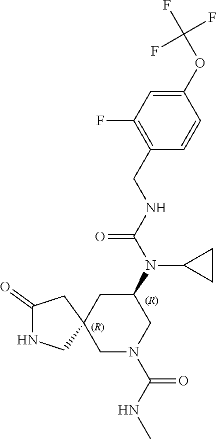 |
| | | (stereoisomer 1 - stereochemistry at spiro |
| | | carbon randomly assigned) |
| 5A | 3 | 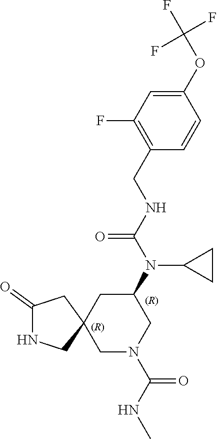 |
| | | (stereoisomer 2 - stereochemistry at spiro |
| | | carbon randomly assigned) |
| 6A | 4 | 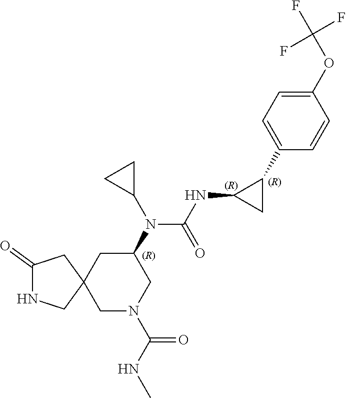 |
| | | (prepared from racemic mixture of trans- |
| | | cyclopropyl stereoisomers) |
| 7A | 5 | 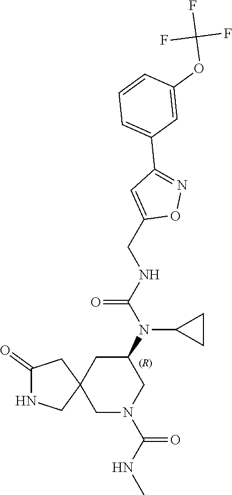 |
| 8A | 6 | 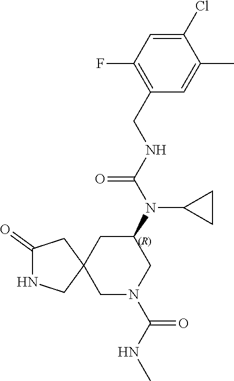 |
| 9A | 7 | 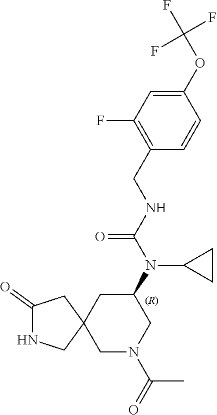 |
| 10A | 8 | 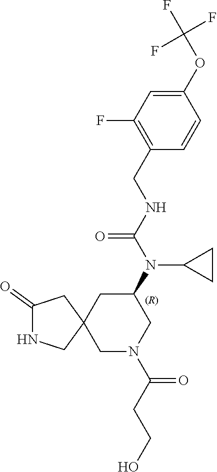 |
| 11A | 5 | 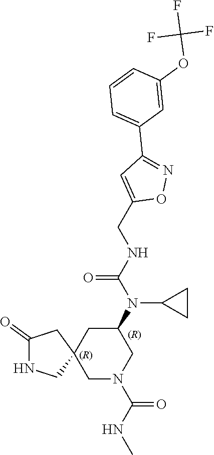 |
| | | (stereoisomer 1 - stereochemistry at spiro |
| | | carbon randomly assigned) |
| 12A | 5 | 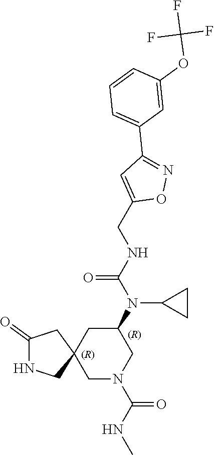 |
| | | (stereoisomer 2 - stereochemistry at spiro |
| | | carbon randomly assigned) |
| 13A | 11 | 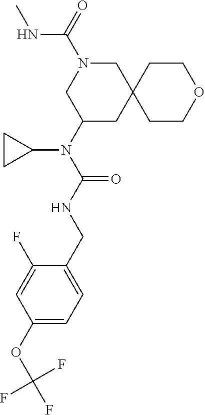 [SMILES: CNC(=O)N1CC(N(C(=O)NCc2ccc(OC(F)(F)F)cc2F)C2CC2)CC2(CCOCC2)C1] |
| 14A | 12 | 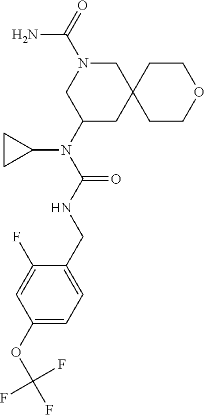 [SMILES: NC(=O)N1CC(N(C(=O)NCc2ccc(OC(F)(F)F)cc2F)C2CC2)CC2(CCOCC2)C1] |

### Synthesis of Common Intermediate A5

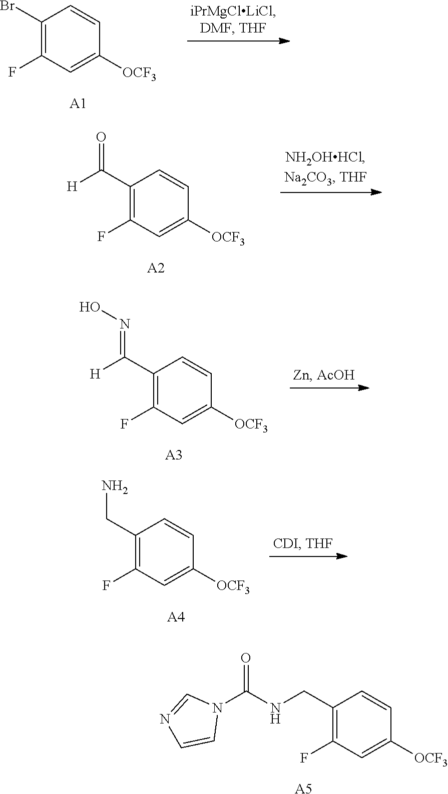

### Step 1: Synthesis of A2

To a solution of A1 (30 g, 116 mmol) in anhydrous THF (250 mL) was added isopropylmagnesium chloride lithium chloride complex (223 mL, 1.3 M in THF) dropwise at −78° C. under N₂ atmosphere. The mixture was stirred at −78° C. for 2 hrs. Then DMF (45 mL, 579 mmol) was added dropwise into the above mixture at −78° C. The resulting mixture was then warmed up to room temperature and stirred for another 1 hr under N₂ atmosphere. After completion, the reaction mixture was quenched with saturated NH₄Cl (200 mL) at 0° C. and extracted with TBME (200 mL×3). The combined organic layers were washed with brine (500 mL), dried over anhydrous Na₂SO₄, filtered and concentrated under reduced pressure to give A2 (24 g, quant.) as brown oil which was used in the next step directly without further purification. LC/MS (ESI) m/z: 209 (M+H)⁺.

### Step 2: Synthesis of A3

To a solution of A2 (24 g, 115 mmol) in THF (400 mL) was added sodium carbonate (24.5 g, 231 mmol) and Hydroxylamine hydrochloride (10.4 g, 150 mmol). The resulting mixture was stirred at 40° C. overnight. Then the mixture was diluted with water (600 mL) and extracted with DCM (400 mL×2). The combined organic layers were washed with brine (400 mL), dried over anhydrous Na₂SO₄, filtered and concentrated to dryness under reduced pressure to give A3 (25.7 g, 99% yield) as white solid which was used in the next step directly without further purification. LC/MS (ESI) m/z: 224 (M+H)⁺.

### Step 3: Synthesis of A4

To a solution of A3 (25.7 g, 115 mmol) in AcOH (250 mL) was added Zn (37.7 g, 576 mmol) in portions. The resulting mixture was stirred at 70° C. for 6 hrs under N₂ atmosphere. After completion, the mixture was basified with aq. NaOH (1 N) to pH=8 and extracted with DCM (400 mL×2). The combined organic layers were washed with brine (400 mL), dried over anhydrous Na₂SO₄, filtered and concentrated to dryness under reduced pressure. The residue was purified via flash column chromatography (eluted with 0˜6% MeOH in DCM) to give A4 (14.1 g, 59% yield) as colorless oil. LC/MS (ESI) m/z: 210 (M+H)⁺.

### Step 4: Synthesis of A5

To a solution of A4 (2.6 g, 12.4 mmol) in TIF (100 mL) was added triethylamine (1.25 g, 12.4 mmol) and CDI (2.2 g, 13.7 mmol) at 0° C. The resulting mixture was stirred at 0° C. for 1 hr under N₂ atmosphere. After completion, the mixture was diluted with water (120 mL) and extracted with DCM (70 mL×2). The combined organic layers were washed with brine (100 mL), dried over anhydrous Na₂SO₄, filtered and concentrated to dryness under reduced pressure. The residue was purified via flash column chromatography (eluted with 0˜20% EtOAc in PE) to give A5 (2.6 g, 69% yield) as white solid. LC/MS (ESI) m/z: 304 (M+H)⁺.

### Synthesis of Example 1/B13

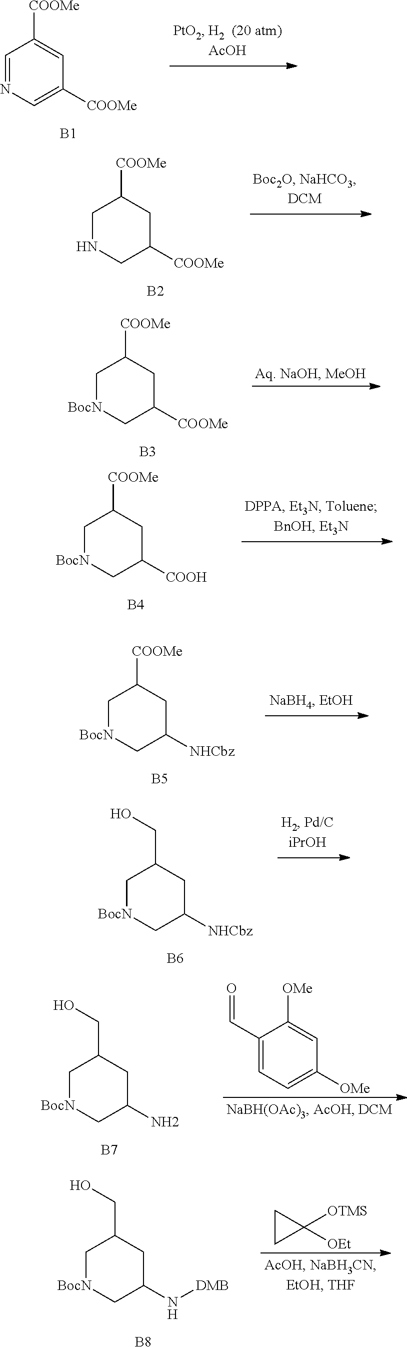

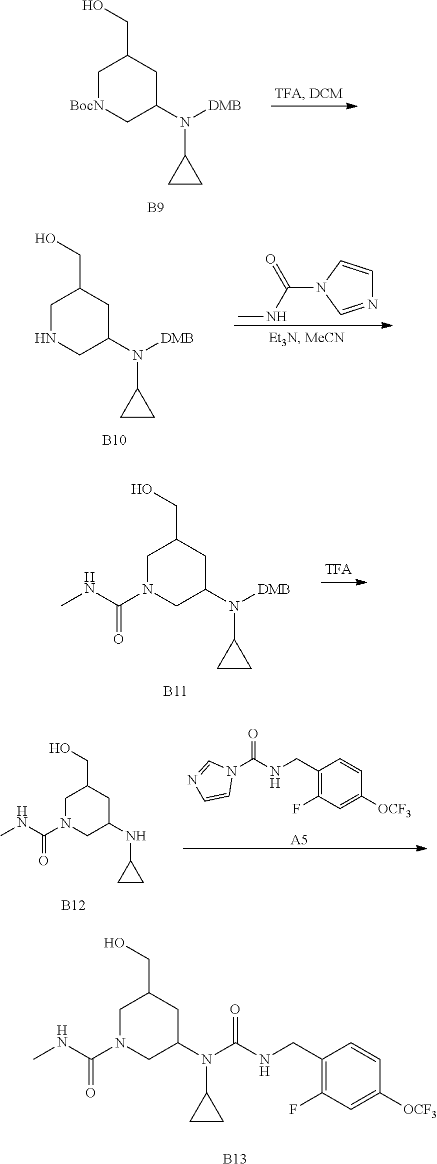

### Step 1: Synthesis of A2

To a solution of B1 (50 g, 256.2 mmol) in AcOH (120 mL) was added PtO₂ (5 g, 22.0 mmol). The resulting mixture was stirred at 50° C. for 16 hrs under H2 atmosphere (20 atm). Then the mixture was filtered and concentrated under reduced pressure to give crude B2 (50 g, 97% yield) which was used in next step directly without further purification. LC/MS (ESI) m/z: 202 (M+H)⁺.

### Step 2: Synthesis of B3

To a solution of B2 (50 g, 248.5 mmol) in DCM (500 mL) were added NaHCO₃ (76 g, 904.7 mmol) and Boc₂O (66.4 g, 304.2 mmol) at 0° C. The resulting mixture was stirred at room temperature for 16 hrs. The mixture was diluted with water (500 mL) and extracted with DCM (500 mL×2). The combined organic layers were washed with brine (500 mL), dried over anhydrous Na₂SO₄, and concentrated under reduced pressure. The residue was purified via flash column chromatography (eluted with 0˜15% EtOAc in PE) to give the cis isomer B3 (24.0 g, 32% yield) as white solid. LC/MS (ESI) m/z: 246 (M+H−56)⁺. ¹H NMR (400 MHz, CDCl₃) δ 4.35 (br s, 2H), 3.69 (s, 6H), 2.88-2.36 (m, 6H), 1.45 (s, 9H); and the trans isomer (9.1 g, 12% yield) as a colorless oil. LC/MS (ESI) m/z: 246 (M+H−56)⁺. ¹H NMR (400 MHz, CDCl₃) δ 3.82-3.70 (m, 2H), 3.68 (s, 6H), 3.60-3.42 (m, 2H), 2.85-2.77 (m, 2H), 2.15-1.96 (m, 2H), 1.44 (s, 9H).

### Step 3: Synthesis of B4

To a solution of B3 (24 g, 79.6 mmol) in MeOH (240 mL) were added 2M NaOH (42 mL, 84.0 mmol, aq.) at 0° C. The resulting mixture was stirred at room temperature for 16 hrs. Then the mixture was diluted with water (250 mL) and extracted with EtOAc (250 mL). The aqueous layer was adjusted with HCl (1M) to pH=4, then extracted with EtOAc (250 mL×2). The combined organic layers were washed with brine (250 mL), dried over anhydrous Na₂SO₄ and concentrated under reduced pressure. The residue was purified via flash column chromatography (eluted with 0˜70% EtOAc in PE) to give B4 (16 g, 70% yield) as white solid. LC/MS (ESI) m/z: 232 (M+H−56)⁺.

### Step 4: Synthesis of B5

To a solution of B4 (16 g, 55.7 mmol) in toluene (160 mL) were added DPPA (18.4 g, 66.8 mmol) and TEA (6.8 g, 66.8 mmol) at 0° C. The resulting mixture was stirred at 110° C. for 2 hrs under N₂ atmosphere. Then BnOH (30.1 g, 278.5 mmol) and TEA (6.8 g, 66.8 mmol) was added into the above mixture at 0° C. The resulting mixture was stirred at 80° C. for 2 hrs. After cooling to room temperature, the mixture was diluted with water (250 mL) and extracted with EtOAc (250 mL×2). The combined organic layers were washed with brine (250 mL), dried over anhydrous Na₂SO₄ and concentrated to dryness under reduced pressure. The residue was purified via flash column chromatography (eluted with 0˜40% EtOAc in PE) to give B5 (12.8 g, 59% yield) as white solid. LC/MS (ESI) m/z: 293 (M+H−100)⁺.

### Step 5: Synthesis of B6

To a solution of B5 (12.8 g, 32.6 mmol) in EtOH (150 mL) was added NaBH₄ (3.0 g, 81.5 mmol) at 0° C. in portions. The resulting mixture was stirred at room temperature for 16 hrs. Then the mixture was quenched with water (200 mL) and extracted with EtOAc (200 mL×2). The combined organic layers were washed with brine (200 mL), dried over anhydrous Na₂SO₄ and concentrated to dryness under reduced pressure. The residue was purified via flash column chromatography (eluted with 0˜60% EtOAc in PE) to give B6 (10.2 g, 28.0 mmol) as white solid. LC/MS (ESI) m/z: 265 (M+H−100)⁺.

### Step 6: Synthesis of B7

To a solution of B6 (10.0 g, 27.5 mmol) in i-PrOH (150 mL) was added Pd/C (1.0 g, 10 wt %) at room temperature under nitrogen atmosphere. The suspension was degassed under vacuum and purged with H₂ several times. The resulting mixture was stirred at room temperature for 18 hrs under H₂ atmosphere. Then the mixture was filtered through a pad of Celite®, the filter cake was washed with MeOH (100 mL). The combined filtrates were concentrated to dryness to give crude B7 (6.1 g, 97% yield) which was used in next step directly without further purification. LC/MS (ESI) m/z: 231 (M+H)⁺.

### Step 7: Synthesis of A8

To a solution of B7 (2.4 g, 10.4 mmol) in DCM (50 mL) were added AcOH (1.2 g, 20.8 mmol) and 2,4-dimethoxybenzaldehyde (8.9 g, 71.5 mmol) at room temperature. The resulting mixture was stirred for 1 hr under N₂ atmosphere. Then NaBH(OAc)₃ (2.65 g, 12.48 mmol) was added into the above mixture in portions at 0° C. and the resulting mixture was stirred at room temperature for 3 hrs under N₂ atmosphere. Then the mixture was filtered and rinsed with DCM (50 mL×2). The filtrate was diluted with water (70 mL) and extracted with DCM (40 mL×2). The combined organic layers were washed with brine (80 mL), dried over anhydrous Na₂SO₄ and concentrated under reduced pressure. The residue was purified via flash column chromatography (eluted with 5˜10% MeOH in DCM) to give B8 (2.7 g, 68% yield) as light-yellow oil. LC/MS (ESI) m/z: 381 (M+H)⁺.

### Step 8: Synthesis of B9

To a solution of B8 (2.7 g, 7.1 mmol) in THF/EtOH (60 mL, v/v=2:1) was added AcOH (4.3 g, 71 mmol), (1-ethoxycyclopropoxy)trimethylsilane (2.5 g, 14.2 mmol) and NaBH₃CN (1.3 g, 21.3 mmol). The resulting mixture was stirred at 80° C. for 4 hrs under N₂ atmosphere. Then the mixture was neutralized with NaHCO₃(aq.) until the pH was adjusted to pH=8. The mixture was diluted with water (80 mL) and extracted with EtOAc (70 mL×2). The combined organic layers were washed with brine (80 mL), dried over anhydrous Na₂SO₄ and concentrated under reduced pressure. The residue was purified via flash column chromatography (eluted with 5˜10% MeOH in DCM) to give B9 (2.0 g, 67% yield) as colorless oil. LC/MS (ESI) m/z: 421 (M+H)⁺.

### Step 9: Synthesis of B10

To a solution of B9 (2.0 g, 4.8 mmol) in DCM (20 mL) was added TFA (4 mL) at 0° C. under N₂ atmosphere. The resulting mixture was stirred at room temperature for 6 hrs. Then the reaction mixture was concentrated under reduced pressure. The residue was diluted with DCM (20 mL), neutralized with NaHCO₃(aq.) until the pH was adjusted to pH=8. Then the mixture was extracted with DCM (40 mL×2). The combined organic layers were washed with brine (40 mL), dried over anhydrous Na₂SO₄ and concentrated under reduced pressure. The residue was purified via flash column chromatography (eluted with 10˜15% MeOH in DCM) to give B10 (1.3 g, 86% yield) as light-yellow oil. LC/MS (ESI) m/z: 321 (M+H)⁺.

### Step 10: Synthesis of B11

To a solution of B10 (680 mg, 2.12 mmol) in MeCN (12 mL) was added TEA (322 mg, 3.18 mmol) and N-methyl-1H-imidazole-1-carboxamide (796 mg, 6.36 mmol) at 0° C. The resulting mixture was heated to 50° C. and stirred for 4 hrs. Then the mixture was diluted with water (30 mL) and extracted with EtOAc (20 mL×2). The combined organic layers were washed with brine (30 mL), dried over anhydrous Na₂SO₄, filtered and concentrated to dryness under reduced pressure. The residue was purified via flash column chromatography (eluted with 0˜50% EtOAc in PE) to give B11 (690 mg, 86% yield) as light-yellow oil. LC/MS (ESI) m/z: 378 (M+H)⁺.

### Step 11: Synthesis of B12

A solution of B11 (690 mg, 1.83 mmol) in TFA (8 mL) was stirred at 80° C. for 4 hrs. Then the reaction mixture was concentrated under reduced pressure to dryness. The residue was dissolved in DCM (20 mL) and neutralized with NaHCO₃(aq. sat.) until the pH was adjusted to pH=8. Then the mixture was diluted with water (30 mL) and extracted with DCM (20 mL×2). The combined organic layers were washed with brine (30 mL), dried over anhydrous Na₂SO₄, filtered and concentrated to dryness under reduced pressure. The residue was purified via flash column chromatography (eluted with 20˜30% MeOH in DCM) to give B12 (470 mg, 98% yield) as light-yellow oil. LC/MS (ESI) m/z: 228 (M+H)⁺.

### Step 3: Synthesis of Example 1/B13

To a solution of B12 (470 mg, 1.80 mmol) in anhydrous THF (15 mL) was added TEA (364 mg, 3.60 mmol) and A5 (546 mg, 1.80 mmol) at 0° C. The resulting mixture was stirred at 50° C. for 18 hrs under N₂ atmosphere. Then the mixture was diluted with water (30 mL) and extracted with EtOAc (20 mL×2). The combined organic layers were washed with brine (30 mL), dried over anhydrous Na₂SO₄, filtered and concentrated to dryness under reduced pressure. The residue was purified via flash column chromatography (eluted with 0˜6% MeOH in DCM) to give rac-Example 1/B13 (292 mg, 35% yield) as white solid. LC/MS (ESI) m/z: 463 (M+H)⁺. This material (200 mg, 0.43 mmol) was further separated via SFC (SHIMADZU PREP SOLUTION SFC; ChiralCel OX, 250×21.2 mm I.D., 5 μm; OZ-M-D-20-8MIN) to afford Example 1/B13 (76 mg, 38% yield, e.e. 100/D) as white solid. ¹H NMR (400 MHz, CD₃OD) δ 7.43 (t, J=8.6 Hz, 1H), 7.09 (t, J=7.8 Hz, 2H), 4.50-4.37 (m, 2H), 4.07 (d, J=9.9 Hz, 1H), 3.88 (d, J=12.4 Hz, 1H), 3.74-3.61 (m, 1H), 3.49-3.39 (m, 2H), 3.08 (s, 1H), 2.70 (s, 3H), 2.59-2.49 (m, 1H), 2.44-2.34 (m, 1H), 1.94-1.78 (m, 2H), 1.77-1.64 (m, 1H), 1.00-0.89 (m, 2H), 0.80-0.68 (m, 2H). ¹⁹F NMR (377 MHz, CD₃OD) δ −59.78 (s), −117.03 (s).

### Synthesis of Example 2/C10

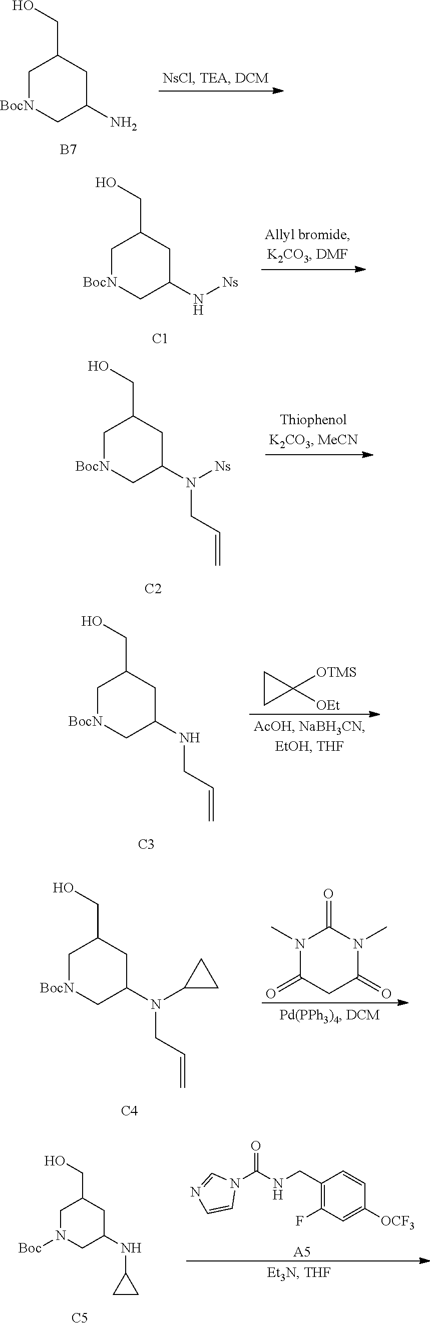

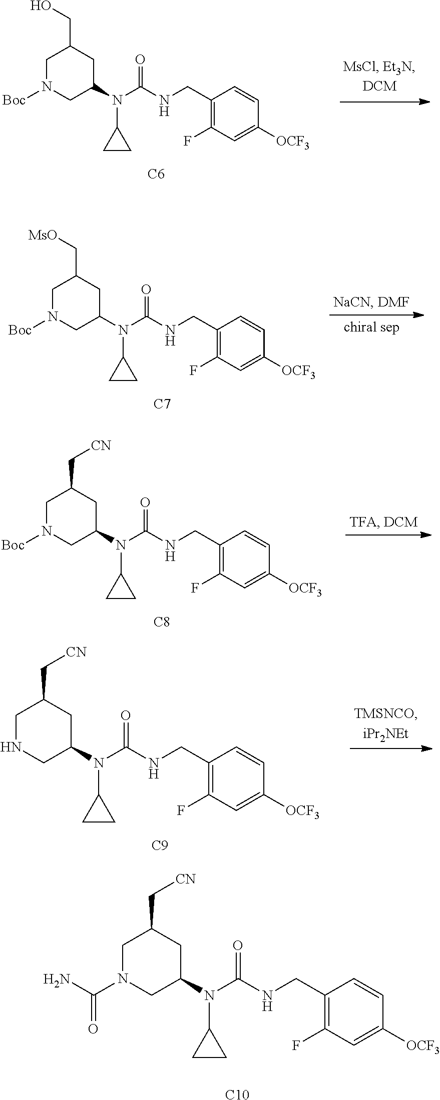

### Step 1: Synthesis of C1

To a solution of B7 (6.1 g, 26.5 mmol) in DCM (100 mL) was added TEA (4.0 g, 40.0 mmol) and NsCl (6.4 g, 29.1 mmol) at 0° C. under N₂ atmosphere. The resulting mixture was stirred at room temperature for 2 hrs. Then the mixture was diluted with water (150 mL) and extracted with DCM (100 mL×2). The combined organic layers were washed with brine (100 mL), dried over anhydrous Na₂SO₄ and concentrated to dryness under reduced pressure. The residue was purified via flash column chromatography (eluted with 5˜10% MeOH in DCM) to give C1 (6.5 g, 59% yield) as white solid. LC/MS (ESI) m/z: 360 (M+H−56)⁺.

### Step 2: Synthesis of C2

To a mixture of C1 (6.5 g, 15.7 mmol) and K₂CO₃ (4.3 g, 31.4 mmol) in DMF (100 mL) was added allyl bromide (3.8 g, 31.4 mmol) in portions at 0° C. and the resulting mixture was stirred at room temperature for 18 hrs under N₂ atmosphere. Then the mixture was filtered and rinsed with DCM (50 mL×2). The filtrate was diluted with water (150 mL) and extracted with DCM (100 mL×2). The combined organic layers were washed with brine (100 mL), dried over anhydrous Na₂SO₄ and concentrated to dryness under reduced pressure. The residue was purified via flash column chromatography (eluted with 0˜5% MeOH in DCM) to give C2 (6.5 g, 91% yield) as a yellow oil. LC/MS (ESI) m/z: 400 (M+H−56)⁺.

### Step 3: Synthesis of C3

To a solution of C2 (6.5 g, 14.3 mmol) in MeCN (120 mL) were added K₂CO₃ (9.9 g, 71.5 mmol) and thiophenol (8.9 g, 71.5 mmol). The resulting mixture was stirred at 80° C. for 18 hrs under N₂ atmosphere. Then the mixture was diluted with water (150 mL) and extracted with DCM (100 mL×2). The combined organic layers were washed with brine (100 mL), dried over anhydrous Na₂SO₄ and concentrated under reduced pressure. The residue was purified via flash column chromatography (eluted with 5˜10% MeOH in DCM) to give C3 (3.1 g, 83% yield) as light-yellow oil. LC/MS (ESI) m/z: 271 (M+H)⁺.

### Step 4: Synthesis of C4

A mixture of C3 (3.1 g, 11.5 mmol), (1-ethoxycyclopropoxy)trimethylsilane (4.0 g, 23.0 mmol), AcOH (6.9 g, 115 mmol) and NaBH₃CN (2.2 g, 34.5 mmol) in a solution of THF/EtOH (90 mL, V/V=2:1) were stirred at 80° C. for 4 hrs under N₂ atmosphere. Then the mixture was neutralized with NaHCO₃(aq., sat. until the pH was adjusted to pH=8. The mixture was diluted with water (60 mL) and extracted with EtOAc (40 mL×2). The combined organic layers were washed with brine (40 mL), dried over anhydrous Na₂SO₄ and concentrated under reduced pressure. The residue was purified via flash column chromatography (eluted with 5˜10% MeOH in DCM) to give C4 (3.2 g, 90% yield) as colorless oil. LC/MS (ESI) m/z: 311 (M+H)⁺.

### Step 5: Synthesis of C5

To a solution of C4 (1.6 g, 5.1 mmol) in DCM (30 mL) was added 1,3-dimethylbarbituric acid (1.2 g, 7.6 mmol) and Pd(PPh₃)₄ (596 mg, 0.5 mmol) at 0° C. under N₂ atmosphere. The resulting mixture was stirred at room temperature for 1 hr. Then the mixture was diluted with water (40 mL) and extracted with DCM (20 mL×2). The combined organic layers were washed with brine (30 mL), dried over anhydrous Na₂SO₄, filtered and concentrated to dryness under reduced pressure. The residue was purified via flash column chromatography (eluted with 0˜10% MeOH in DCM) to give C5 (1.2 g, 85% yield) as yellow oil. LC/MS (ESI) m/z: 271 (M+H)⁺.

### Step 6: Synthesis of C6

To a solution of C5 (1.2 g, 4.4 mmol) in anhydrous THF (30 mL) was added TEA (897 mg, 8.8 mmol) and A5 (22 mg, 0.12 mmol) at 0° C. The resulting mixture was stirred at 60° C. for 3 hrs under N₂ atmosphere. Then the mixture was diluted with water (40 mL) and extracted with EtOAc (30 mL×2). The combined organic layers were washed with brine (30 mL), dried over anhydrous Na₂SO₄, filtered and concentrated to dryness under reduced pressure. The residue was purified via flash column chromatography (eluted with 0˜5% MeOH in DCM) to give C6 (1.23 g, 54% yield) as colorless oil. LC/MS (ESI) m/z: 506 (M+H)⁺.

### Step 7: Synthesis of C7

To a solution of C6 (1.23 g, 2.4 mmol) in anhydrous DCM (30 mL) were added TEA (737 mg, 7.2 mmol) and MsCl (334 mg, 2.8 mmol) at 0° C. The resulting mixture was stirred at 0° C. for 2 hrs under N₂ atmosphere. Then the mixture was quenched with water (40 mL) and extracted with DCM (30 mL×2). The combined organic layers were washed with brine (30 mL), dried over anhydrous Na₂SO₄, filtered and concentrated to dryness under reduced pressure. The residue was purified via flash column chromatography (eluted with 0˜5% MeOH in DCM) to give C7 (1.2 g, 84% yield) as colorless oil. LC/MS (ESI) m/z: 584 (M+H)⁺.

### Step 8: Synthesis of C8

To a solution of C7 (1.2 g, 2.0 mmol) in DMF (16 mL) was added NaCN (121 mg, 2.4 mmol) at room temperature. The resulting mixture was stirred at 90° C. for 2 hrs under N₂ atmosphere. Then the mixture was diluted with saturated NH₄Cl solution (50 mL) and extracted with EtOAc (30 mL×2). The combined organic layers were washed with brine (50 mL), dried over anhydrous Na₂SO₄, filtered and concentrated to dryness under reduced pressure. The residue was purified via flash column chromatography (eluted with 0˜5% MeOH in DCM) to give C8 (1.0 g, 95% yield) as white solid. LC/MS (ESI) m/z: 515 (M+H)⁺. C8 (1.0 g, 1.9 mmol) was further separated via SFC (Waters Thar 80 preparative SFC; ChiralPak AD, 250×4.6 mm I.D. 5 μm; AD_MeOH_DEA_40) to afford C8-P1 (390 mg, 39% yield, e.e. 99%) and C8-P2 (450 mg, 45% yield, e.e. 99%) as white solid. LC/MS (ESI) m/z: 515 (M+H)⁺.

### Step 9: Synthesis of C9

To a solution of C8-P2 (40 mg, 78 μmol) in DCM (3 mL) was added TFA (1 mL) dropwise at 0° C. The resulting mixture was stirred at 0° C. for 1 hr under N₂ atmosphere. Then the mixture was concentrated under reduced pressure to give crude C9 (30 mg, 93% yield) as purple oil, which was used in the next step directly without further purification. LC/MS (ESI) m/z: 415 (M+H)⁺.

### Step 10: Synthesis of Example 2/C10

To a mixture of C9 (30 mg, 72 μmol) and DIEA (19 mg, 0.14 mmol) in anhydrous THF (5 mL) was added TMSNCO (12 mg, 0.1 mmol) dropwise at 0° C. The resulting mixture was stirred at room temperature for 16 hrs under N₂ atmosphere. Then the mixture was diluted with water (30 mL) and extracted with EtOAc (20 mL×2). The combined organic layers were washed with brine (20 mL), dried over anhydrous Na₂SO₄, filtered and concentrated to dryness under reduced pressure. The residue was purified via prep-HPLC to give Example 2/C10 (18 mg, 54% yield) as white solid. LC/MS (ESI) m/z: 458 (M+H)⁺. ¹H NMR (400 MHz, MeOD) δ 7.46-7.40 (m, 1H), 7.12-7.05 (m, 2H), 6.98 (t, J=5.9 Hz, 1H), 4.47-4.40 (m, 2H), 4.15-4.05 (m, 1H), 3.93-3-83 (m, 1H), 3.74-3.64 (m, 1H), 3.25-3.17 (m, 1H), 2.59-2.54 (m, 1H), 2.52-2.41 (m, 3H), 2.08-1.96 (m, 2H), 1.95-1.86 (m, 1H), 0.99-0.91 (m, 2H), 0.81-0.73 (m, 2H). ¹⁹F NMR (377 MHz, MeOD) δ −59.78 (s), −117.01 (s).

### Synthesis of Example 3/D5

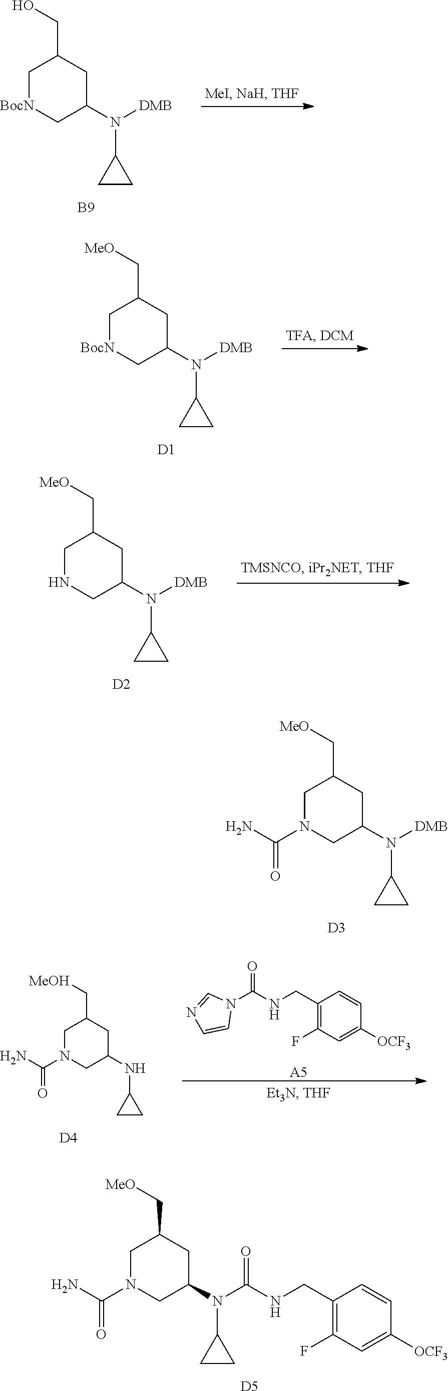

### Step 1: Synthesis of D1

To a solution of B9 (365 mg, 0.87 mmol) in anhydrous DMF (8 mL) was added NaH (32 mg, 1.3 mmol) in portions at 0° C. under N₂ atmosphere. The mixture was stirred at 0° C. for 30 min and then Mel (71 mg, 0.96 mmol) was added into the above mixture at 0° C. dropwise. The resulting mixture was allowed to warm to room temperature and stirred for 4 hrs. Then the mixture was quenched with saturated NH₄Cl (20 mL) and extracted with EtOAc (20 mL×2). The combined organic layers were washed with brine (20 mL), dried over anhydrous Na₂SO₄, filtered and concentrated to dryness under reduced pressure. The residue was purified via flash column chromatography (eluted with 30˜35% EtOAc in PE) to give D1 (299 mg, 79% yield) as colorless oil. LC/MS (ESI) m/z: 435 (M+H)⁺.

### Step 2: Synthesis of D2

To a solution of D1 (299 mg, 0.69 mmol) in DCM (8 mL) was added TFA (2 mL) dropwise at 0° C. The resulting mixture was stirred at room temperature for 1 hr. Then the mixture was concentrated under reduced pressure to give crude D2 (205 mg, 89% yield) as purple oil which was used in the next step directly without further purification. LC/MS (ESI) m/z: 335 (M+H)⁺.

### Step 3: Synthesis of D3

To a mixture of D2 (205 mg, 0.61 mmol) and DIEA (155 mg, 1.2 mmol) in anhydrous DCM (10 mL) was added TMSNCO (91 mg, 0.79 mmol) dropwise at 0° C. under N₂ atmosphere. The resulting mixture was stirred at room temperature for 2 hrs. Then the mixture was diluted with water (20 mL) and extracted with DCM (20 mL×2). The combined organic layers were washed with brine (20 mL), dried over anhydrous Na₂SO₄, filtered and concentrated to dryness under reduced pressure. The residue was purified via flash column chromatography (eluted with 20˜25% MeOH in DCM) to give D3 (177 mg, 76% yield) as light-yellow oil. LC/MS (ESI) m/z: 378 (M+H)⁺.

### Step 4: Synthesis of D4

A round-bottom flask was charged with D3 (177 mg, 0.47 mmol) and TFA (4 mL), the reaction mixture was stirred at 80° C. for 2 hrs under N₂ atmosphere. Then the mixture was concentrated under reduced pressure to give crude D4 (104 mg, quant.) as purple oil which was used in the next step directly without further purification. LC/MS (ESI) m/z: 228 (M+H)⁺.

### Step 5: Synthesis of Example 3/D5

To a mixture of D4 (104 mg, 0.46 mmol) and TEA (70 mg, 0.69 mmol) in anhydrous THF (6 mL) was added A5 (139 mg, 0.46 mmol) at 0° C. The resulting mixture was stirred at 60° C. for 4 hrs. Then the mixture was diluted with H₂O (20 mL) and extracted with EtOAc (20 mL×2). The combined organic layers were washed with brine (20 mL), dried over anhydrous Na₂SO₄, filtered and concentrated to dryness under reduced pressure. The residue was purified via prep-HPLC to give rac-Example 3/D5 (146 mg, 69% yield) as white solid. LC/MS (ESI) m/z: 463 (M+H)⁺. The material (146 mg, 0.32 mmol) was further separated via SFC (Waters Thar 80 preparative SFC; ChiralPak IB, 100×4.6 mm I.D. 5 μm; IB_EtOH_DEA_20) to afford Example 3/D5 (46 mg, 32% yield, e.e. 100%) as white solid. ¹H NMR (400 MHz, MeOD) δ 7.44 (t, J=8.6 Hz, 1H), 7.09 (t, J=7.7 Hz, 2H), 4.44 (s, 2H), 4.07 (d, J=12.3 Hz, 1H), 3.91 (d, J=11.6 Hz, 1H), 3.68 (d, J=4.1 Hz, 1H), 3.33 (s, 3H), 3.30-3.24 (m, 2H), 3.21-3.12 (m, 1H), 2.59-2.51 (m, 1H), 2.49-2.39 (m, 1H), 1.89 (d, J=7.2 Hz, 3H), 0.97-0.91 (m, 2H), 0.81-0.70 (m, 2H). ¹⁹F NMR (377 MHz, MeOD) δ −59.78 (s), −117.04 (s).

### Synthesis of Example 4/E14

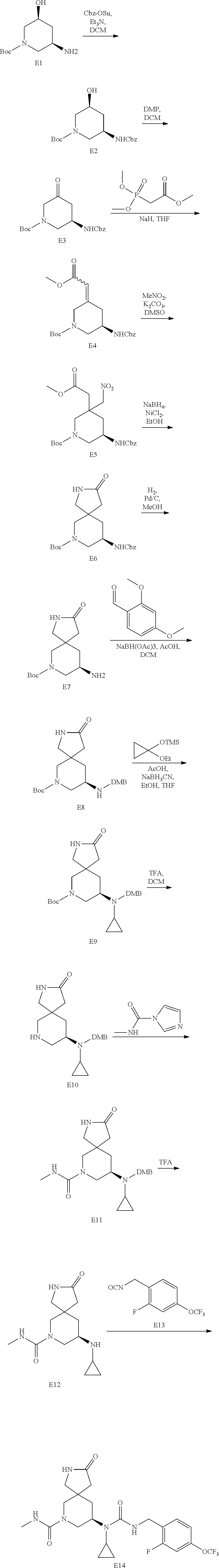

### Step 1: Synthesis of E2

To a mixture of TEA (16 mL, 116 mmol) and E1 (10.0 g, 46.2 mmol) in DCM (100 mL) was added Cbz-OSu (17.3 g, 69.4 mmol) in portions at 0° C. The resulting mixture was warmed up to room temperature and stirred for 20 hrs. The mixture was diluted with H₂O (300 mL) and extracted with DCM (400 mL×2). The combined organic layers were washed with brine (200 mL), dried over anhydrous Na₂SO₄, filtered and concentrated to dryness under reduced pressure. The residue was purified via flash column chromatography on silica gel (DCM:MeOH=100:0 to 100:3) to give E2 (15.9 g, 98% yield) as white solid. LC/MS (ESI) m/z: 295 (M+H−56)⁺.

### Step 2: Synthesis of E3

To a solution of E2 (8.0 g, 22.8 mmol) in anhydrous DCM (80 mL) was added Dess-Martin periodinane (19.4 g, 45.7 mmol) in portions at 0° C. The resulting mixture was stirred at room temperature for 2 hrs. Then the mixture was quenched with saturated NaHCO₃(aq.) (200 mL) and extracted with DCM (100 mL×2). The combined organic layers were washed with brine (150 mL), dried over anhydrous Na₂SO₄, filtered and concentrated to dryness under reduced pressure. The residue was purified via flash column chromatography on silica gel (DCM:MeOH=100:0 to 100:2) to give E3 (7.9 g, 99% yield) as white solid. LC/MS (ESI) m/z: 249 (M+H−100)⁺.

### Step 3: Synthesis of E4

To a solution of Trimethyl phosphonoacetate (4.0 mL, 27.2 mmol) in anhydrous THF (100 mL) was added NaH (1.1 g, 27.2 mmol) in portions at 0° C., the reaction mixture was stirred at 0° C. for 30 mins under N₂ atmosphere. Then a solution of E3 (7.9 g, 22.7 mmol) in THF (50 mL) was added into the above mixture at 0° C. dropwise. The resulting mixture was warmed up to room temperature and stirred for additional 16 hrs. Then the mixture was quenched with saturated NH₄Cl (200 mL) and extracted with EtOAc (150 mL×2). The combined organic layers were washed with brine (150 mL), dried over anhydrous Na₂SO₄, filtered and concentrated to dryness under reduced pressure. The residue was purified via flash column chromatography on silica gel (PE:EtOAc=100:0 to 100:35) to give E4 (7.2 g, 79% yield) as colorless oil. LC/MS (ESI) m/z: 304 (M+H−100)⁺.

### Step 4: Synthesis of E5

To a mixture of E4 (7.2 g, 17.8 mmol) and K₂CO₃ (2.5 g, 17.8 mmol) in DMSO (100 mL) was added nitromethane (10.8 g, 178 mmol). The resulting mixture was stirred at 100° C. for 16 hrs under N₂ atmosphere. Then the mixture was diluted with H₂O (300 mL) and extracted with EtOAc (200 mL×2). The combined organic layers were washed with brine (200 mL), dried over anhydrous Na₂SO₄, filtered and concentrated to dryness under reduced pressure. The residue was purified via flash column chromatography on silica gel (PE:EtOAc=100:0 to 100:25) to give E5 (3.8 g, 46% yield) as colorless oil. LC/MS (ESI) m/z: 366 (M+H−100)⁺.

### Step 5: Synthesis of E6

To a solution of E5 (3.8 g, 8.0 mmol) in EtOH (100 mL) was added Nickel chloride (10.4 g, 80.1 mmol) and NaBH₄ (3.0 g, 80.1 mmol) in portions at 0° C. The resulting mixture was stirred at room temperature for 2 hrs. Then the mixture was quenched with saturated NH₄Cl (aq) (150 mL) and extracted with EtOAc (100 mL×2). The combined organic layers were washed with brine (150 mL), dried over anhydrous Na₂SO₄, filtered and concentrated to dryness under reduced pressure. The residue was purified via flash column chromatography on silica gel (DCM:MeOH=100:0 to 100:4) to give E6 (2.2 g, 68% yield) as colorless oil. LC/MS (ESI) m/z: 304 (M+H−100)⁺.

### Step 6: Synthesis of E7

To a solution of E6 (2.2 g, 5.5 mmol) in MeOH (30 mL) was added Pd/C (10 wt %) (440 mg) and the mixture was degassed under N₂ atmosphere for three times. The resulting mixture was stirred at room temperature for 2 hrs under H2 atmosphere with 20 psi. Then the mixture was diluted with DCM (100 mL) and filtered through a pad of Celite®, the filter cake was washed with MeOH (25 mL). The combined filtrates were dried over anhydrous Na₂SO₄, filtered and concentrated to dryness to give crude E7 (1.4 g, 95% yield) as colorless oil which was used in the next step directly without further purification. LC/MS (ESI) m/z: 270 (M+H)⁺.

### Step 7: Synthesis of E8

To a mixture of E7 (1.4 g, 5.2 mmol) and 2,4-dimethoxybenzaldehyde (860 mg, 5.2 mmol) in DCM (50 mL) was added AcOH (614 mg, 10.4 mmol) and the mixture was stirred at room temperature for 1 hr. Then the reaction mixture was cooled down to 0° C. and NaBH(OAc)₃ (3.3 g, 15.5 mmol) was added into the above mixture in portions. The resulting mixture was stirred at room temperature for 16 hrs. After completion, the reaction mixture was concentrated to dryness under reduced pressure. The residue was purified via flash column chromatography on silica gel (eluted with DCM:MeOH=100:0 to 100:10) to give E8 (2.1 g, 96% yield) as colorless oil. LC/MS (ESI) m/z: 420 (M+H)⁺.

### Step 8: Synthesis of E9

To a mixture of E8 (2.1 g, 5.0 mmol) and AcOH (3 mL, 54.4 mmol) in EtOH (20 mL) and THF (40 mL) was added (1-ethoxycyclopropoxy)trimethylsilane (3 mL, 13.6 mmol) and NaBH₃CN (1.2 g, 19.0 mmol). The resulting mixture was stirred at 80° C. for 3 hrs under N₂ atmosphere. Then the mixture was concentrated to dryness under reduced pressure. The residue was dissolved in DCM (80 mL) and washed with saturated NaHCO₃(aq.) (80 mL). The organic layer was separated, washed with brine (100 mL), dried over anhydrous Na₂SO₄, filtered and concentrated to dryness under reduced pressure. The residue was purified by column chromatography on silica gel (DCM:MeOH=100:0 to 100:5) to give E9 (2.1 g, 91% yield) as colorless oil. LC/MS (ESI) m/z: 460 (M+H)⁺.

### Step 9: Synthesis of E10

To a solution of E9 (2.1 g, 4.6 mmol) in DCM (20 mL) was added TFA (5 mL) dropwise at 0° C. and the resulting mixture was stirred at room temperature for 1 hr. After completion, the reaction mixture was concentrated under reduced pressure to give crude E10 (1.6 g, quant.) as colorless oil which was used in the next step directly without further purification. LC/MS (ESI) m/z: 360 (M+H)⁺.

### Step 10: Synthesis of E11

To a mixture of E10 (1.6 g, 4.5 mmol) and DIEA (3.0 mL, 20.8 mmol) in anhydrous MeCN (80 mL) was added N-methyl-1H-imidazole-1-carboxamide (3.3 g, 26.0 mmol). The resulting mixture was stirred at 60° C. for 16 hrs. Then the mixture was diluted with H₂O (150 mL) and extracted with EtOAc (80 mL×2). The combined organic layers were washed with brine (150 mL), dried over anhydrous Na₂SO₄ and concentrated to dryness under reduced pressure. The residue was purified via flash column chromatography on silica gel (eluted with DCM:MeOH=100:0 to 100:8) to give E11 (1.6 g, 86% yield) as colorless oil. LC/MS (ESI) m/z: 417 (M+H)⁺.

### Step 11: Synthesis of E12

A round-bottom flask was charged with Eli (1.6 g, 3.8 mmol) and TFA (20 mL), the reaction mixture was stirred at 80° C. for 4 hrs under N₂ atmosphere. Then the mixture was concentrated under reduced pressure to give crude E12 (980 mg, quant.) as purple oil which was used in the next step directly without further purification. LC/MS (ESI) m/z: 267 (M+H)⁺.

### Step 12: Synthesis of Example 4/E14

To a mixture of E12 (310 mg, 1.2 mmol) and TEA (0.7 mL, 4.8 mmol) in anhydrous DCM (12 mL) was added a solution of E13 (282 mg, 1.2 mmol) in DCM (5 mL) dropwise at 0° C. under N₂ atmosphere. The resulting mixture was stirred at room temperature for 30 mins. Then the mixture was diluted with H₂O (30 mL) and extracted with DCM (20 mL×2). The combined organic layers were washed with brine (30 mL), dried over anhydrous Na₂SO₄, filtered and concentrated to dryness under reduced pressure. The residue was purified via flash column chromatography on silica gel (eluted with DCM:MeOH=100:0 to 100:15) to afford rac-Example 4/E14 (330 mg). LC/MS (ESI) m/z: 502 (M+H)⁺. This sample was further separated via SFC (ChiralCel OZ, 250×21.2 mm I.D., 5 μm; mobile phase: A for C02 and B for MeOH+0.1% NH₃H₂O; B %: 40%-40%, 4.0 min; 120 min) to give Example 4/E14 (98 mg, 17% yield) as white solid. ¹H NMR (400 MHz, MeOD) δ 7.49-7.41 (m, 1H), 7.16-7.07 (m, 2H), 4.51-4.39 (m, 2H), 4.09-4.02 (m, 1H), 3.97-3.88 (m, 1H), 3.80-3.67 (m, 1H), 3.22-3.14 (m, 3H), 2.72 (s, 3H), 2.66-2.60 (m, 1H), 2.59-2.53 (m, 1H), 2.38-2.31 (m, 1H), 2.30-2.21 (m, 2H), 1.94-1.86 (m, 1H), 1.02-0.95 (m, 2H), 0.82-0.75 (m, 2H). ¹⁹F NMR (377 MHz, MeOD) δ −59.77 (s), −116.97 (s).

### Synthesis of Example 5/F17

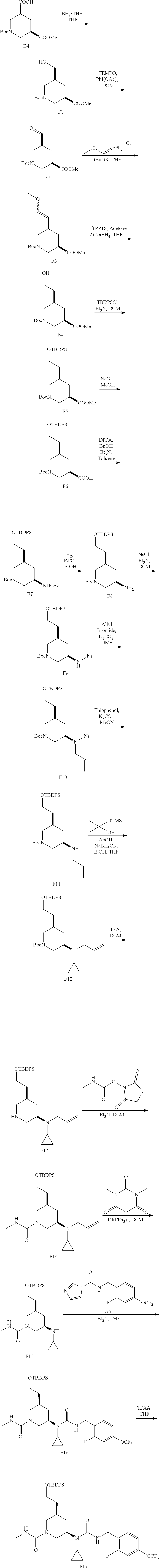

### Step 2: Synthesis of F1

To a solution of B4 (7.5 g, 26.1 mmol) in anhydrous THF (100 mL) was added BH₃-THF (65.3 mL, 1 M in THF) dropwise at 0° C. under N₂ atmosphere. The resulting mixture was stirred at room temperature for 2 hrs. Then the mixture was poured into ice-water (150 mL) slowly and extracted with EtOAc (100 ml, x 2). The combined organic layers were washed with brine (200 mL), dried over anhydrous Na₂SO₄, filtered and concentrated to dryness under reduced pressure. The residue was purified via flash column chromatography (eluted with 0˜5% MeOH in DCM) to give F1 (4.8 g, 67% yield) as colorless oil. LC/MS (ESI) m/z: 274 (M+H)⁺.

### Step 3: Synthesis of F2

To a solution of F1 (4.8 g, 17.6 mmol) in DCM (60 mL) was added TEMPO (375 mg, 1.76 mmol)

and PhI(OAc)₂ (8.54 g, 26.3 mmol) at 0° C. The resulting mixture was stirred at room temperature for 4 hrs. Then the mixture was diluted with water (120 mL) and extracted with DCM (80 mL×2). The combined organic layers were washed with brine (150 mL), dried over anhydrous Na₂SO₄, filtered and concentrated to dryness under reduced pressure. The residue was purified via flash column chromatography (eluted with 0˜40% EA in PE to give F2 (4.3 g, 90% yield) as yellow oil. LC/MS (ESI) m/z: 272 (M+H)⁺.

### Step 4: Synthesis of F3

To a mixture of Methoxymethylenetriphenylphosphonium chloride (5.6 g, 16.4 mmol) in anhydrous THF (70 mL) was added t-BuOK (15.3 mL, 1 M in THF) at 0° C. and the mixture was stirred at 0° C. for 30 mins under N₂ atmosphere. Then a solution of F2 (2.96 g, 10.9 mmol) in THF (70 mL) was added into the above mixture at 0° C. under N₂ atmosphere. The resulting mixture was stirred at room temperature for another 1 hr. Then the mixture was diluted with water (120 mL) and extracted with EtOAc (70 mL×2). The combined organic layers were washed with brine (120 mL), dried over anhydrous Na₂SO₄, filtered and concentrated to dryness under reduced pressure. The residue was purified via flash column chromatography (eluted with 0˜10% EA in PE to give F3 (2.5 g, 76% yield) as yellow oil. LC/MS (ESI) m/z: 300 (M+H)⁺.

### Step 5: Synthesis of F4

To a solution of F3 (3.9 g, 13 mmol) in acetone (60 mL) was added PPTS (6.55 g, 26 mmol).

The resulting mixture was stirred at 50° C. for 24 hrs. Then the mixture was diluted with water (120 mL) and extracted with EtOAc (70 mL×2). The combined organic layers were washed with brine (120 mL), dried over anhydrous Na₂SO₄, filtered and concentrated to dryness under reduced pressure. This residue was dissolved in THF (60 mL) and NaBH₄ (696 mg, 18.4 mmol) was added into the above solution in portions at 0° C. The resulting mixture was stirred at 0° C. for 20 mins. Then the mixture was quenched with water (100 mL) and extracted with EtOAc (70 mL×2). The combined organic layers were washed with brine (100 mL), dried over anhydrous Na₂SO₄, filtered and concentrated to dryness. The residue was purified via flash column chromatography (eluted with 0-4% MeOH in DCM) to give F4 (2.7 g, 76% yield) as yellow oil. LC/MS (ESI) m/z: 288 (M+H)⁺.

### Step 6: Synthesis of F5

To a mixture of F4 (2.7 g, 9.4 mmol) and TEA (3.9 mL, 28.2 mmol) in DCM (50 mL) was added DMAP (574 mg, 4.7 mmol) and TBDPSCl (3.2 mL, 12.2 mmol) at 0° C. The resulting mixture was stirred at room temperature for 18 hrs. Then the mixture was diluted with water (120 mL) and extracted with DCM (80 mL×2). The combined organic layers were washed with brine (120 mL), dried over anhydrous Na₂SO₄, filtered and concentrated to dryness under reduced pressure. The residue was purified via flash column chromatography (eluted with 0-7% EtOAc in PE to give F5 (3.4 g, 69% yield) as colorless oil. LC/MS (ESI) m/z: 526 (M+H)⁺.

### Step 7: Synthesis of F6

To a solution of F5 (3.4 g, 6.5 mmol) in MeOH (50 mL) was added 2 M NaOH (6.5 mL, 13 mmol, aq.) at 0° C. The resulting mixture was stirred at room temperature for 2 hrs. Then the mixture was diluted with water (100 mL) and extracted with TBME (100 mL). The aqueous layer was separated, adjusted with aq. HCl (1 N) to pH=4 and extracted with EtOAc (100 mL×2). The combined organic layers were washed with brine (150 mL), dried over anhydrous Na₂SO₄, filtered and concentrated under reduced pressure to give crude F6 (2.4 g, 70% yield) as white solid which was used in the next step directly without further purification. LC/MS (ESI) m/z: 512 (M+H)⁺.

### Step 8: Synthesis of F7

To a solution of F6 (2.4 g, 4.7 mmol) in toluene (50 mL) was added DPPA (1.3 mL, 6.1 mmol) and TEA (1.96 mL, 14.1 mmol) at 0° C. The resulting mixture was stirred at 110° C. for 2 hrs under N₂ atmosphere. Then BnOH (1.45 mL, 14.1 mmol) was added into the above mixture at 0° C. The resulting mixture was stirred at 90° C. for another 6 hrs under N₂ atmosphere. After cooling to room temperature, the mixture was diluted with water (120 mL) and extracted with EtOAc (70 mL×2). The combined organic layers were washed with brine (120 mL), dried over anhydrous Na₂SO₄, filtered and concentrated to dryness under reduced pressure. The residue was purified via flash column chromatography (eluted with 0˜18% EtOAc in PE) to give F7 (1.6 g, 55% yield) as white solid. LC/MS (ESI) m/z: 517 (M+H−100)⁺.

### Step 9: Synthesis of F8

To a solution of F7 (1.6 g, 2.6 mmol) in i-PrOH (40 mL) was added Pd/C (0.4 g, 10 wt %) at room temperature, the resulting mixture was stirred at room temperature for 4 hrs under H2 atmosphere with 20 psi. Then the mixture was filtered through a pad of Celite®, the filter cake was washed with MeOH (40 mL). The combined filtrates were concentrated to dryness to give crude F8 (1.2 g, 96% yield) which was used in the next step directly without further purification. LC/MS (ESI) m/z: 483 (M+H)⁺.

### Step 10: Synthesis of F9

To a mixture of F8 (1.2 g, 2.5 mmol) and TEA (1 mL, 7.5 mmol) in anhydrous DCM (50 mL) was added NsCl (580 mg, 2.63 mmol) in portions at 0° C. under N₂ atmosphere. The resulting mixture was stirred at room temperature for 7 hrs. Then the mixture was diluted with water (80 mL) and extracted with DCM (50 mL×2). The combined organic layers were washed with brine (80 mL), dried over anhydrous Na₂SO₄, filtered and concentrated to dryness under reduced pressure. The residue was purified via flash column chromatography (eluted with 0˜22% EtOAc in PE) to give F9 (1.4 g, 84% yield) as yellow solid. LC/MS (ESI) m/z: 668 (M+H)⁺.

### Step 11: Synthesis of F10

To a mixture of F9 (1.4 g, 2.1 mmol) and K₂CO₃ (869 mg, 6.3 mmol) in DMF (40 mL) was added allyl bromide (0.27 mL, 3.1 mmol) dropwise at 0° C. and the resulting mixture was stirred at room temperature for 18 hrs under N₂ atmosphere. Then the mixture was diluted with water (80 mL) and extracted with EtOAc (50 mL×2). The combined organic layers were washed with brine (80 mL), dried over anhydrous Na₂SO₄, filtered and concentrated to dryness under reduced pressure. The residue was purified via flash column chromatography (eluted with 0˜18% EtOAc in PE) to give F10 (1.4 g, 94% yield) as yellow oil. LC/MS (ESI) m/z: 652 (M+H−56)⁺.

### Step 12: Synthesis of F11

To a solution of F10 (1.4 g, 1.84 mmol) in MeCN (50 mL) was added K₂CO₃ (2.54 g, 18.4 mmol) and thiophenol (1.1 g, 9.2 mmol). The resulting mixture was stirred at 70° C. for 18 hrs under N₂ atmosphere. Then the mixture was diluted with water (80 mL) and extracted with DCM (50 mL×2). The combined organic layers were washed with brine (100 mL), dried over anhydrous Na₂SO₄, filtered and concentrated to dryness under reduced pressure. The residue was purified via flash column chromatography (eluted with 0˜10% MeOH in DCM) to give F11 (875 mg, 91% yield) as light-yellow oil. LC/MS (ESI) m/z: 523 (M+H)⁺.

### Step 13: Synthesis of F12

A mixture of F11 (875 mg, 1.67 mmol) and AcOH (0.96 mL, 16.7 mmol) in THF/EtOH (48 mL, v/v=2:1) was added (1-ethoxycyclopropoxy)trimethylsilane (870 mg, 5.01 mmol) and NaBH₃CN (316 mg, 5.01 mmol). The resulting mixture was stirred at 80° C. for 4 hrs under N₂ atmosphere. Then the mixture was basified with NaHCO₃ solution (aq., sat.) until the pH was adjusted to pH=8. The mixture was diluted with water (80 mL) and extracted with EtOAc (40 mL×2). The combined organic layers were washed with brine (80 mL), dried over anhydrous Na₂SO₄, filtered and concentrated to dryness under reduced pressure. The residue was purified via flash column chromatography (eluted with 0-3% MeOH in DCM) to give F12 (730 mg, 77% yield) as colorless oil. LC/MS (ESI) m/z: 563 (M+H)⁺.

### Step 14: Synthesis of F13

To a solution of F12 (330 mg, 0.6 mmol) in DCM (10 mL) was added TFA (2 mL) dropwise at 0° C. under N₂ atmosphere. The resulting mixture was stirred at room temperature for 2 hrs. Then the reaction mixture was concentrated under reduced pressure to give crude F13 (270 mg, quant.) as brown oil which was used in the next step directly without further purification. LC/MS (ESI) m/z: 463 (M+H)⁺.

### Step 15: Synthesis of F14

To a mixture of F13 (270 mg, 0.58 mmol) and TEA (0.24 mL, 1.75 mmol) in anhydrous DCM (15 mL) was added 2,5-dioxopyrrolidin-1-yl methylcarbamate (150 mg, 0.87 mmol) dropwise at 0° C. under N₂ atmosphere. The resulting mixture was stirred at room temperature for 1 hr. Then the mixture was diluted with water (50 mL) and extracted with DCM (30 mL×2). The combined organic layers were washed with brine (50 mL), dried over anhydrous Na₂SO₄, filtered and concentrated under reduced pressure to dryness. The residue was purified via flash column chromatography (eluted with 0˜5% MeOH in DCM) to give F14 (235 mg, 77% yield) as colorless oil. LC/MS (ESI) m/z: 520 (M+H)⁺.

### Step 16: Synthesis of F15

To a solution of F14 (235 mg, 0.45 mmol) in DCM (14 mL) were added 1,3-Dimethylbarbituric acid (141 mg, 0.9 mmol) and Pd(PPh₃)₄ (104 mg, 0.09 mmol) at 0° C. The resulting mixture was stirred at room temperature for 2 hrs under N₂ atmosphere. Then the mixture was concentrated to give crude F15 (226 mg, quant.) as orange oil which was used in the next step directly without further purification. LC/MS (ESI) m/z: 480 (M+H)⁺.

### Step 17: Synthesis of F16

To a mixture of F15 (226 mg, 0.47 mmol) and TEA (0.2 mL, 1.41 mmol) in THF (15 mL) was added A5 (150 mg, 0.5 mmol) at room temperature under N₂ atmosphere, the resulting mixture was stirred at 60° C. for 4 hrs. Then the mixture was diluted with water (40 mL) and extracted with EtOAc (20 mL×2). The combined organic layers were washed with brine (40 mL), dried over anhydrous Na₂SO₄, filtered and concentrated to dryness under reduced pressure. The residue was purified via flash column chromatography (eluted with 0-3% MeOH in DCM) to give F16 (315 mg, 94% yield) as white solid. LC/MS (ESI) m/z: 737 (M+Na)⁺.

### Step 18: Synthesis of Example 5/F17

To a solution of 21 (315 mg, 0.44 mmol) in THF (15 mL) was added TBAF (230 mg, 0.88 mmol). The resulting mixture was stirred at room temperature for 1 hr. Then the mixture was diluted with water (50 mL) and extracted with EtOAc (20 mL×2). The combined organic layers were washed with brine (40 mL), dried over anhydrous Na₂SO₄, filtered and concentrated under reduced pressure to dryness. The residue was purified via flash column chromatography (eluted with 0-8% MeOH in DCM) to give rac-Example 5 (155 mg, 74% yield) as white solid. LC/MS (ESI) m/z: 477 (M+H)⁺. The material (155 mg, 0.33 mmol) was further separated via SFC (Waters Thar 80 preparative SFC; ChiralCel OX, 250×20 mm I.D., 5 μm; 40 mL/min) to afford Example 5/(65 mg, 31% yield, e.e. 99%). ¹H NMR (400 MHz, CD₃OD) δ 7.43 (t, J=8.6 Hz, 1H), 7.09 (t, J=7.6 Hz, 2H), 4.44 (d, J=4.7 Hz, 2H), 4.02-3.94 (m, 1H), 3.91-3.82 (m, 1H), 3.69-3.58 (m, 3H), 3.16-3.08 (m, 1H), 2.69 (d, J=3.9 Hz, 3H), 2.58-2.50 (m, 1H), 2.33 (dd, J=13.1, 11.4 Hz, 1H), 1.95 (d, J=12.1 Hz, 1H), 1.79 (q, J=11.9 Hz, 1H), 1.71-1.60 (m, 1H), 1.58-1.37 (m, 2H), 1.00-0.88 (m, 2H), 0.81-0.70 (m, 2H). ¹⁹F NMR (376 MHz, CD₃OD) δ −59.71-−59.83 (m), −117.01 (d, J=4.2 Hz).

### Synthesis of Example 6/G12

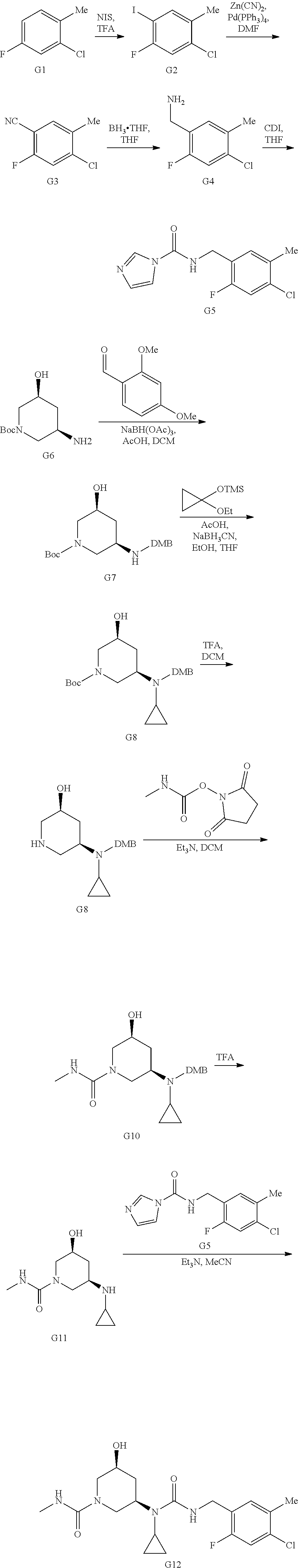

### Step 1: Synthesis of G2

To a solution of G2 (30.0 g, 207.6 mmol) in TFA (300 mL) was added NIS (46.7 g, 207.6 mmol) at 0° C. in portions. The resulting mixture was stirred at room temperature for 16 hours. Then the mixture was concentrated under reduced pressure, the residue was dissolved in DCM (600 mL) and washed with aq. NaHCO₃ (400 mL*2) and brine (400 mL), dried over anhydrous Na₂SO₄, filtered and concentrated under reduced pressure to dryness. The residue was purified via flash column chromatography on silicagel (eluted with PE:EtOAc=100:0 to 95:5) to give G2 (36.0 g, 64% yield) as white solid. ¹H NMR (400 MHz, MeOD) δ 7.72 (dd, J=6.8, 0.4 Hz, 1H), 7.19 (d, J=7.8 Hz, 1H), 2.30 (s, 3H).

### Step 2: Synthesis of G3

To a solution of G2 (27 g, 99.8 mmol) in anhydrous DMF (270 mL) was added Zn(CN)₂ (12.9 g, 109.8 mmol) followed by the addition of Pd(PPh₃)₄ (5.7 g, 4.9 mmol). The resulting mixture was stirred at 100° C. for 16 hours under N₂ atmosphere. After cooling to room temperature, the mixture was diluted with EtOAc (400 mL) and filtered. The filtrate was washed with saturated aq. NH₄Cl (200 mL) twice. Then the organic layer was separated, washed with brine (200 mL), dried over anhydrous Na₂SO₄, filtered and concentrated to dryness under reduced pressure to dryness. The residue was purified via flash column chromatography on silica gel (eluted with PE:EtOAc=100:0 to 95:5) to give G3 (10.0 g, 59.1% yield) as white solid.

### Step 3: Synthesis of G4

To a solution of G3 (10 g, 58.9 mmol) in anhydrous THF (100 mL) was added BH₃-THF (295 mL, 1M in THF) dropwise at 0° C. The resulting mixture was stirred at room temperature for 16 hours under N₂ atmosphere. LCMS showed the starting material was consumed completely. Then the mixture was quenched with MeOH (80 mL) dropwise and concentrated to dryness under reduced pressure. The residue was dissolved in EtOAc (150 mL) and washed with aq. HCl (100 mL, 1 N). The aqueous phase was separated, basified with 15% aq. NaOH to pH=10. Then the mixture was extracted with EtOAc (100 mL*3), the combined organic layers were separated, washed with brine (100 mL), dried over Na₂SO₄, filtered and concentrated under reduced pressure to give crude G4 (5.5 g, 53.4% yield) as colorless oil which was used directly in the next step without further purification. LCMS: ESI m/z: 174 (M+H)⁺.

### Step 4: Synthesis of G5

To a solution of G4 (36 mg, 0.21 mmol) in THF (3 mL) was added CDI (36 mg, 0.22 mmol) at 0° C. The resulting mixture was stirred at room temperature for 50 mins. Then the mixture was concentrated under reduced pressure to give crude G5 (55 mg, 98.99% yield) as colorless oil which was used in the next step directly without further purification. LC/MS (ESI) m/z: 268 (M+H)⁺.

### Step 5: Synthesis of G7

To a solution of G6 (400 mg, 1.85 mmol) in DCM (20 mL) was added 2,4-dimethoxybenzaldehyde (338 mg, 2.04 mmol), NaBH(OAc)₃ (785 mg, 3.70 mmol) and AcOH (cat.). The resulting mixture was stirred at room temperature for 16 hrs under N₂ atmosphere. Then the mixture was diluted with water (30 mL) and extracted with DCM (20 mL×2). The combined organic layers were washed with brine (30 mL), dried over anhydrous Na₂SO₄, filtered and concentrated to dryness under reduced pressure. The residue was purified via flash column chromatography (eluted with 0˜10% MeOH in DCM) to give G7 (550 mg, 81% yield) as white solid. LC/MS (ESI) m/z: 367 (M+H)⁺.

### Step 6: Synthesis of G8

To a mixture of G7 (550 mg, 1.50 mmol) and AcOH (1.35 g, 22.5 mmol) in THF/EtOH (30 mL, v/v=1:1) was added (1-ethoxycyclopropoxy)trimethylsilane (915 mg, 5.26 mmol) and NaBH₃CN (284 mg, 4.51 mmol). The resulting mixture was stirred at 80° C. for 2 hrs under N₂ atmosphere. The mixture was basified with saturated NaHCO₃ solution to pH=8 and extracted with EtOAc (30 mL×2). The combined organic layers were washed with brine (40 mL), dried over anhydrous Na₂SO₄, filtered and concentrated to dryness under reduced pressure. The residue was purified via flash column chromatography (eluted with 0-100% EtOAc in PE) to give G8 (450 mg, 74% yield) as white solid. LC/MS (ESI) m/z: 407 (M+H)⁺.

### Step 7: Synthesis of G9

To a solution of G8 (450 mg, 1.11 mmol) in DCM (20 mL) was added TFA (4 mL) dropwise at 0° C. The resulting mixture was stirred at room temperature for 2 hrs under N₂ atmosphere. Then the mixture was basified with saturated NaHCO₃ solution to adjust pH=8 and extracted with DCM (20 mL×2). The combined organic layers were washed with brine (30 mL), dried over anhydrous Na₂SO₄, filtered and concentrated to dryness under reduced pressure. The residue was purified via flash column chromatography (eluted with 0˜10% MeOH in DCM) to give G9 (285 mg, 84% yield) as yellow oil. LC/MS (ESI) m/z: 307 (M+H)⁺.

### Step 8: Synthesis of G10

To a solution of G9 (285 mg, 0.93 mmol) in DCM (15 mL) were added TEA (282 mg, 2.79 mmol) and 2,5-dioxopyrrolidin-1-yl methylcarbamate (320 mg, 1.86 mmol) at 0° C. dropwise.

The resulting mixture was stirred at room temperature for 16 hrs under N₂ atmosphere. Then the mixture was diluted with water (30 mL) and extracted with DCM (20 mL×2). The combined organic layers were washed with brine (30 mL), dried over anhydrous Na₂SO₄, filtered and concentrated to dryness under reduced pressure. The residue was purified via flash column chromatography (eluted with 0˜10% MeOH in DCM) to give G10 (250 mg, 74% yield) as white solid. LC/MS (ESI) m/z: 364 (M+H)⁺.

### Step 9: Synthesis of G11

A solution of G10 (250 mg, 0.69 mmol) in TFA (10 mL) was stirred at 80° C. for 2 hrs under N₂ atmosphere. Then the mixture was basified with saturated NaHCO₃ solution to adjust pH=8 and extracted with DCM (20 mL×2). The combined organic layers were washed with brine (30 mL), dried over anhydrous Na₂SO₄, filtered and concentrated to dryness under reduced pressure. The residue was purified via flash column chromatography (eluted with 0˜10% MeOH in DCM) to give G11 (120 mg, 82% yield) as yellow oil. LC/MS (ESI) m/z: 214 (M+H)⁺.

### Step 10: Synthesis of Example 6/G12

To a mixture of G11 (40 mg, 0.19 mmol) and TEA (57 mg, 056 mmol) in MeCN (10 mL) was added G5 (150 mg, 0.28 mmol) at room temperature. The resulting mixture was stirred at 60° C. for 16 hrs under nitrogen atmosphere. Then the mixture was quenched with NH₄Cl (20 mL, sat., aq.) and extracted with EtOAc (15 mL×2). The combined organic layers were washed with brine (20 mL), dried over anhydrous Na₂SO₄, filtered and concentrated to dryness under reduced pressure. The residue was purified via Prep-HPLC to give Example 6/G12 (8 mg, 10% yield) as white solid. LC/MS (ESI) m/z: 413 (M+H)⁺. ¹H NMR (400 MHz, MeOD) δ 7.24 (d, J=8.0 Hz, 1H), 7.13 (d, J=9.8 Hz, 1H), 6.90 (t, J=5.9 Hz, 1H), 4.42-4.31 (m, 2H), 4.17-4.06 (m, 1H), 3.83-3.74 (m, 1H), 3.73-3.63 (m, 1H), 3.60-3.45 (m, 1H), 3.11-3.02 (m, 1H), 2.69 (s, 3H), 2.55-2.48 (m, 1H), 2.46-2.36 (m, 1H), 2.32 (s, 3H), 2.14 (d, J=11.4 Hz, 1H), 2.09-1.97 (m, 1H), 1.00-0.88 (m, 2H), 0.80-0.70 (m, 2H). ¹⁹F NMR (376 MHz, MeOD) δ −123.11 (s).

### Synthesis of Example 7/H1

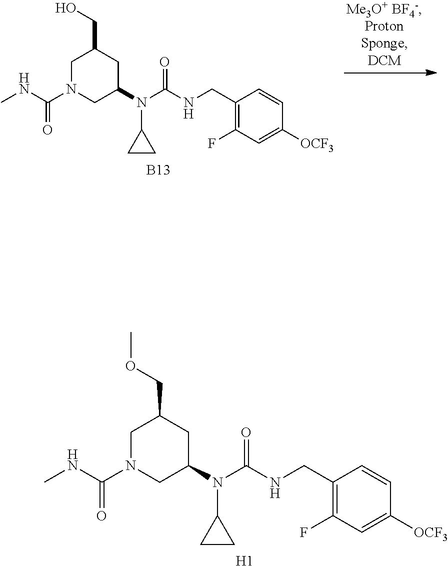

### Step 1: Synthesis of Example 7/H1

To a solution of rac-B13 (50 mg, 0.11 mmol) in anhydrous DCM (6 mL) was added Meerwein's salt (24 mg, 0.16 mmol) and proton sponge (46.4 mg, 0.22 mmol) at 0° C. The resulting mixture was stirred at room temperature for 48 hrs under N₂ atmosphere. Then the mixture was diluted with water (30 mL) and extracted with DCM (20 mL×2). The combined organic layers were washed with brine (30 mL), dried over anhydrous Na₂SO₄, filtered and concentrated to dryness. The residue was purified via flash column chromatography (eluted with 0˜10% MeOH in DCM) to give rac-Example 7/H1 (13 mg, 25% yield) as white solid. LC/MS (ESI) m/z: 477 (M+H)⁺. The material (13 mg, 0.027 mmol) was further separated via SFC (SHIMADZU PREP SOLUTION SFC; ChiralCel OZ, 250×21.2 mm I.D., 5 μm; OZ-M-D-20-8MIN) to afford Example 7/H1 (4.0 mg, 31% yield, e.e. 100%) as white solid. ¹H NMR (400 MHz, CD₃OD) δ 7.43 (t, J=8.6 Hz, 1H), 7.09 (t, J=7.8 Hz, 2H), 4.43 (s, 2H), 4.05 (d, J=14.0 Hz, 1H), 3.88 (dd, J=12.5, 4.0 Hz, 1H), 3.74-3.58 (m, 1H), 3.32 (s, 3H), 3.28 (t, J=5.5 Hz, 2H), 3.16-3.02 (m, 1H), 2.70 (s, 3H), 2.57-2.50 (m, 1H), 2.40 (dd, J=13.3, 10.8 Hz, 1H), 1.97-1.75 (m, 3H), 0.97-0.91 (m, 2H), 0.79-0.71 (m, 2H). ¹⁹F NMR (377 MHz, CD₃OD) δ −59.77 (s), −117.01 (s).

### Synthesis of Example 8/I1

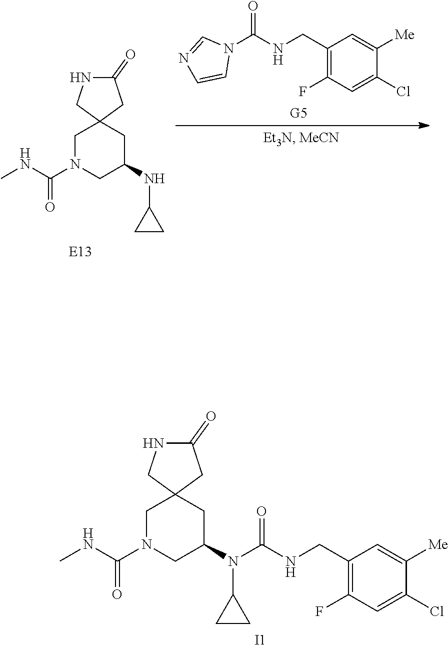

### Step 1: Synthesis of Example 8/H1

To a mixture of E13 (42 mg, 0.16 mmol) and TEA (162 mg, 1.60 mmol) in THF (6 mL) was added G5 (55 mg, 0.21 mmol) at 0° C. The resulting mixture was stirred at 60° C. for 16 hrs under N₂ atmosphere. Then the mixture was diluted with H₂O (30 mL) and extracted with EtOAc (20 mL×2). The combined organic layers were washed with brine (30 mL), dried over anhydrous Na₂SO₄, filtered and concentrated to dryness under reduced pressure. The residue was purified via prep-HPLC to give Example 8/I1 (20.7 mg, 28% yield) as white solid. LC/MS (ESI) m/z: 466 (M+H)⁺. ¹H NMR (400 MHz, MeOD) δ 7.26 (d, J=8.0 Hz, 1H), 7.15 (d, J=9.8 Hz, 1H), 6.98-6.88 (m, 1H), 4.46-4.33 (m, 2H), 4.12-4.01 (m, 1H), 3.96-3.86 (m, 1H), 3.82-3.68 (m, 1H), 3.32-3.30 (m, 0.5H), 3.24-3.18 (m, 1H), 3.18-3.15 (m, 1H), 3.15-3.09 (m, 0.5H), 2.72 (d, J=0.9 Hz, 3H), 2.66-2.57 (m, 1H), 2.57-2.48 (m, 1H), 2.39-2.34 (m, 0.5H), 2.33 (s, 3H), 2.31-2.21 (m, 2H), 2.17-2.10 (m, 0.5H), 2.00-1.86 (m, 1H), 1.01-0.91 (m, 2H), 0.82-0.72 (m, 2H). ¹⁹F NMR (376 MHz, MeOD) δ −123.07 (d, J=4.4 Hz).

### Synthesis of Example 9/J10

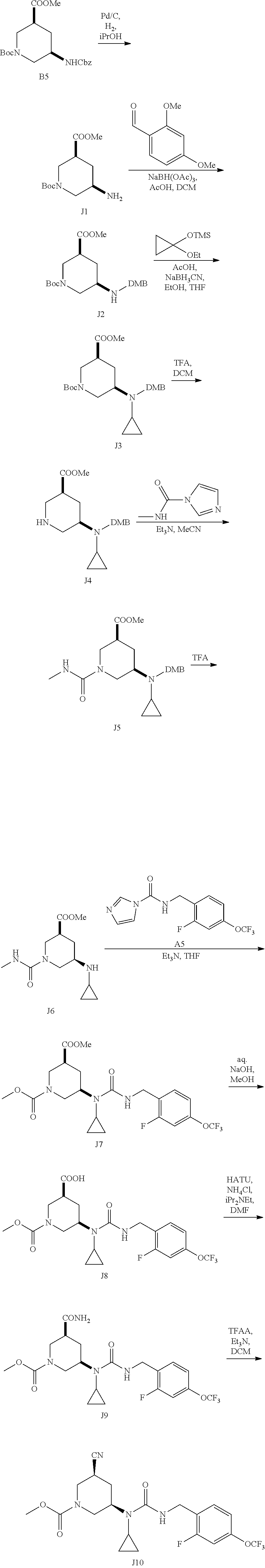

### Step 1: Synthesis of J1

To a solution of B5 (1.3 g, 3.3 mmol) in i-PrOH (30 mL) was added Pd/C (260 mg, 10 wt %) under nitrogen atmosphere. The suspension was degassed under vacuum and purged with H2 several times. The resulting mixture was stirred at room temperature for 16 hrs under H2 atmosphere with 20 psi. Then the mixture was filtered through a pad of Celite®, the filter cake was washed with MeOH (30 mL). The combined filtrates were concentrated to dryness to give crude J1 (840 mg, 98% yield) as yellow oil which was used in the next step directly without further purification. LC/MS (ESI) m/z: 259 (M+H)⁺.

### Step 2: Synthesis of J2

To a solution of J1 (840 mg, 3.3 mmol) in DCM (20 mL) was added AcOH (396 mg, 6.6 mmol) and 2,4-dimethoxybenzaldehyde (531 mg, 3.2 mmol). The resulting mixture was stirred at room temperature for 1 hr under N₂ atmosphere. Then NaBH(OAc)₃ (2.1 g, 9.9 mmol) was added into the above mixture in portions at 0° C. and the resulting mixture was stirred at room temperature for another 3 hrs under N₂ atmosphere. Then the mixture was filtered and rinsed with DCM (30 mL×2). The filtrate was diluted with water (50 mL) and extracted with DCM (30 mL×2). The combined organic layers were washed with brine (50 mL), dried over anhydrous Na₂SO₄, filtered and concentrated to dryness under reduced pressure. The residue was purified via flash column chromatography (eluted with 5˜10% MeOH in DCM) to give J2 (1.1 g, 83% yield) as light-yellow oil. LC/MS (ESI) m/z: 409 (M+H)⁺.

### Step 3: Synthesis of J3

To a mixture of J2 (1.1 g, 2.7 mmol) and AcOH (1.6 g, 27 mmol) in THF/EtOH (48 mL, v/v=2:1) was added (1-ethoxycyclopropoxy)trimethylsilane (940 mg, 5.4 mmol) and NaBH₃CN (509 mg, 8.1 mmol). The resulting mixture was stirred at 80° C. for 4 hrs under N₂ atmosphere. Then the mixture was neutralized with NaHCO₃(aq.) until the pH was adjusted to pH=8 and extracted with EtOAc (50 mL×2). The combined organic layers were washed with brine (80 mL), dried over anhydrous Na₂SO₄, filtered and concentrated to dryness under reduced pressure. The residue was purified via flash column chromatography (eluted with 25˜35% EtOAc in PE) to give J3 (830 mg, 69% yield) as colorless oil. LC/MS (ESI) m/z: 449 (M+H)⁺.

### Step 4: Synthesis of J4

To a solution of J3 (830 mg, 1.85 mmol) in DCM (20 mL) was added TFA (4 mL) dropwise at 0° C. The resulting mixture was stirred at room temperature for 1 hr under N₂ atmosphere. Then the mixture was concentrated under reduced pressure to give crude J4 (572 mg, 89% yield) as purple oil which was used in the next step directly without further purification. LC/MS (ESI) m/z: 349 (M+H)⁺.

### Step 5: Synthesis of J5

To a mixture of J4 (572 mg, 1.6 mmol) and TEA (646 mg, 6.4 mmol) in anhydrous DCM (20 mL) was added N-methyl-1H-imidazole-1-carboxamide (263 mg, 2.1 mmol) dropwise at 0° C. under N₂ atmosphere. The resulting mixture was stirred at room temperature for 12 hrs. Then the mixture was diluted with water (50 mL) and extracted with DCM (30 mL×2). The combined organic layers were washed with brine (40 mL), dried over anhydrous Na₂SO₄, filtered and concentrated under reduced pressure to dryness. The residue was purified via flash column chromatography (eluted with 5˜10% MeOH in DCM) to give J5 (606 mg, 91% yield) as colorless oil. LC/MS (ESI) m/z: 406 (M+H)⁺.

### Step 6: Synthesis of J6

A round-bottom flask was charged with J5 (606 mg, 1.5 mmol) and TFA (5 mL), the reaction mixture was stirred at 80° C. for 2 hrs under N₂ atmosphere. Then the mixture was concentrated under reduced pressure to give crude J6 (350 mg, quant.) as purple oil which was used in next step directly without further purification. LC/MS (ESI) m/z: 256 (M+H)⁺.

### Step 7: Synthesis of J7

To a mixture of J6 (350 mg, 1.37 mmol) and TEA (208 mg, 2.06 mmol) in anhydrous THF (10 mL) was added A5 (415 mg, 1.37 mmol) at 0° C. The resulting mixture was stirred at 60° C. for 4 hrs under N₂ atmosphere. Then the mixture was quenched with water (50 mL) and extracted with EtOAc (30 mL×2). The combined organic layers were washed with brine (50 mL), dried over anhydrous Na₂SO₄, filtered and concentrated to dryness under reduced pressure. The residue was purified via flash column chromatography (eluted with 10˜15% MeOH in DCM) to give J7 (534 mg, 79% yield) as colorless oil. LC/MS (ESI) m/z: 491 (M+H)⁺.

### Step 8: Synthesis of J8

To a solution of J7 (534 mg, 1.1 mmol) in MeOH (10 mL) was added 2M NaOH (1.1 mL, 2.2 mmol, aq.) at 0° C. The resulting mixture was stirred at room temperature for 2 hrs. Then the mixture was diluted with water (40 mL) and extracted with TBME (30 mL). The aqueous layer was separated, adjusted with aq. HCl (1 N) to pH=5 and extracted with EtOAc (30 mL×2). The combined organic layers were washed with brine (50 mL), dried over anhydrous Na₂SO₄, filtered and concentrated to dryness under reduced pressure. The residue was purified via flash column chromatography (eluted with 0˜80% EtOAc in PE) to give J8 (460 mg, 89% yield) as white solid. LC/MS (ESI) m/z: 477 (M+H)⁺.

### Step 9: Synthesis of J9

To a solution of J8 (460 mg, 0.97 mmol) in DMF (15 mL) was added DIEA (250 mg, 1.94 mmol) and HATU (380 mg, 1.1 mmol). The resulting mixture was stirred at room temperature for 15 min under N₂ atmosphere. Then NH₄Cl (81 mg, 1.5 mmol) was added into the above mixture in portions at 0° C. and the resulting mixture was stirred at room temperature for another 6 hrs under N₂ atmosphere. Then the mixture was quenched with NH₄Cl (60 mL) and extracted with EtOAc (30 mL×2). The combined organic layers were washed with brine (50 mL), dried over anhydrous Na₂SO₄, filtered and concentrated to dryness under reduced pressure. The residue was purified via flash column chromatography (eluted with 15˜20% MeOH in DCM) to give J9 (402 mg, 88% yield) as light-yellow oil. LC/MS (ESI) m/z: 476 (M+H)⁺.

### Step 10: Synthesis of Example 9/J10

To a mixture of 12 (402 mg, 0.85 mmol) and TEA (172 mg, 1.7 mmol) in anhydrous DCM (15 mL) was added TFAA (273 mg, 1.3 mmol) dropwise at 0° C. and the resulting mixture was stirred at room temperature for another 3 hrs under N₂ atmosphere. Then the mixture was diluted with water (50 mL) and extracted with DCM (30 mL×2). The combined organic layers were washed with brine (30 mL), dried over anhydrous Na₂SO₄, filtered and concentrated to dryness under reduced pressure. The residue was purified via prep-HPLC to give rac-Example 9/J10 (220 mg, 57% yield) as white solid. LC/MS (ESI) m/z: 458 (M+H)⁺. The material was further separated via SFC (Waters Thar 80 preparative SFC; ChiralPak IG, 100×4.6 mm I.D. 5 μm; IG_MeOH_DEA_20) to afford Example 9/J10 (63 mg, 29% yield, e.e. 100%) as white solid. ¹H NMR (400 MHz, MeOD) δ 7.43 (t, J=8.6 Hz, 1H), 7.10 (t, J=7.7 Hz, 2H), 4.44 (s, 2H), 4.29 (d, J=9.4 Hz, 1H), 3.85 (d, J=9.3 Hz, 1H), 3.63-3.51 (m, 1H), 3.26 (s, 1H), 2.90-2.79 (m, 2H), 2.70 (s, 3H), 2.62-2.52 (m, 1H), 2.48-2.34 (m, 1H), 2.30-2.20 (m, 1H), 1.04-0.94 (m, 2H), 0.83-0.73 (m, 2H). ¹⁹F NMR (377 MHz, MeOD) δ −59.78 (s), −116.98 (s).

### Synthesis of Example 10/K4

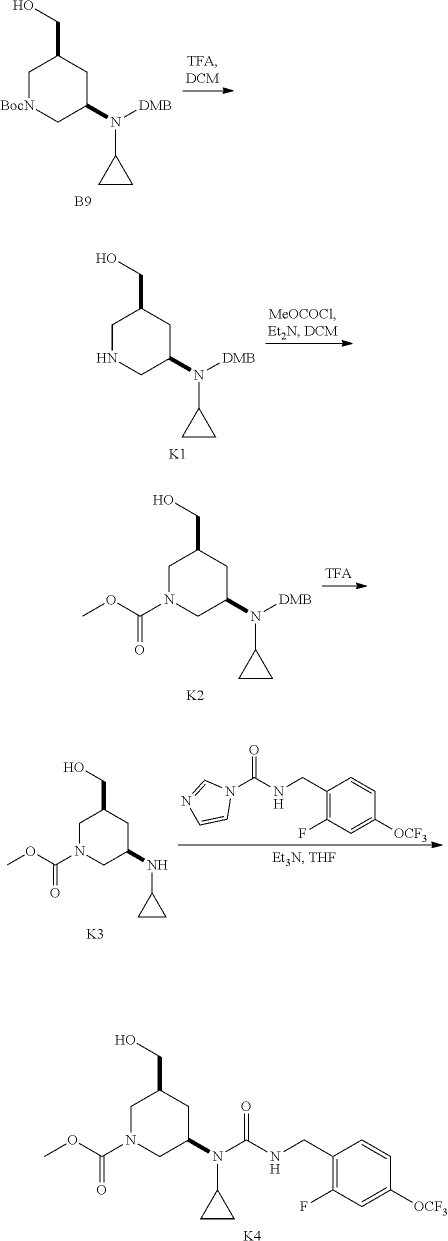

### Step 1: Synthesis of K1

To a solution of B9 (1.5 g, 3.57 mmol) in DCM (25 mL) was added TFA (5 mL) dropwise at 0° C. The resulting mixture was stirred at room temperature for 5 hrs under N₂ atmosphere.

Then the mixture was concentrated under reduced pressure to dryness. The residue was dissolved in DCM (30 mL) and basified with saturated NaHCO₃ solution until the pH was adjusted to pH=8. The resulting mixture was diluted with H₂O (80 mL) and extracted with DCM (50 mL×2). The combined organic layers were washed with brine (80 mL), dried over anhydrous Na₂SO₄, filtered and concentrated to dryness under reduced pressure. The residue was purified via flash column chromatography (eluted with 10˜20% MeOH in DCM) to give K1 (1.13 g, 99/yield) as light-yellow oil. LC/MS (ESI) m/z: 321 (M+H)⁺.

### Step 2: Synthesis of K2

To a mixture of K1 (1.13 g, 3.53 mmol) and TEA (714 mg, 7.06 mmol) in dry DCM (30 mL) was added methyl chloroformate (334 mg, 3.53 mmol) dropwise at 0° C. under N₂ atmosphere. The resulting mixture was stirred at room temperature for 2 hrs. Then the mixture was diluted with water (50 mL) and extracted with DCM (30 mL×2). The combined organic layers were washed with brine (50 mL), dried over anhydrous Na₂SO₄, filtered and concentrated to dryness under reduced pressure. The residue was purified via flash column chromatography (eluted with 5˜10% MeOH in DCM) to give K2 (1.32 g, 99% yield) as colorless oil. LC/MS (ESI) m/z: 379 (M+H)⁺.

### Step 3: Synthesis of K3

A solution of K2 (400 mg, 1.06 mmol) in TFA (6 mL) was stirred at 80° C. for 3 hrs under N₂ atmosphere. After completion, the mixture was concentrated to dryness under reduced pressure to dryness. The residue was dissolved in DCM (20 mL) and basified with saturated NaHCO₃ solution until the pH was adjusted to pH=8. The resulting mixture was diluted with H₂O (50 mL) and extracted with DCM (30 mL×2). The combined organic layers were washed with brine (50 mL), dried over anhydrous Na₂SO₄, filtered and concentrated to dryness under reduced pressure. The residue was purified via flash column chromatography (eluted with 10˜20% MeOH in DCM) to give K3 (190 mg, 79% yield) as colorless oil. LC/MS (ESI) m/z: 229 (M+H)⁺.

### Step 4: Synthesis of Example 10/K4

To a mixture of K3 (190 mg, 0.83 mmol) and TEA (168 mg, 1.66 mmol) in anhydrous THF (12 mL) was added A5 (265 mg, 0.83 mmol) at 0° C. The resulting mixture was stirred at 50° C. for 18 hrs under N₂ atmosphere. Then the mixture was diluted with water (50 mL) and extracted with EtOAc (30 mL×2). The combined organic layers were washed with brine (50 mL), dried over anhydrous Na₂SO₄, filtered and concentrated to dryness under reduced pressure. The residue was purified via flash column chromatography (eluted with 0˜10% MeOH in DCM) to give rac-Example 10/K4 (160 mg, 42% yield) as white solid. LC/MS (ESI) m/z: 464 (M+H)⁺. The material was further separated via SFC (SHIMADZU PREP SOLUTION SFC; ChiralCel OX, 250×21.2 mm I.D., 5 μm; IC_EtOH_DEA_30_8 min) to afford Example 10/K4 (55 mg, 34% yield, e.e. 100%) as white solid. ¹H NMR (400 MHz, CD₃OD) δ 7.42 (t, J=8.6 Hz, 1H), 7.09 (t, J=8.4 Hz, 2H), 4.43 (s, 2H), 4.26-4.16 (m, 1H), 4.10-3.98 (m, 1H), 3.76-3.66 (m, 1H), 3.68 (s, 3H), 3.48-3.36 (m, 2H), 3.17 (t, J=12.2 Hz, 1H), 2.58-2.32 (m, 2H), 1.92-1.80 (m, 2H), 1.76-1.68 (m, 1H), 0.99-0.87 (m, 2H), 0.80-0.68 (m, 2H). ¹⁹F NMR (377 MHz, CD₃OD) δ −59.78 (s), −117.08 (s).

### Synthesis of Example 11/L8

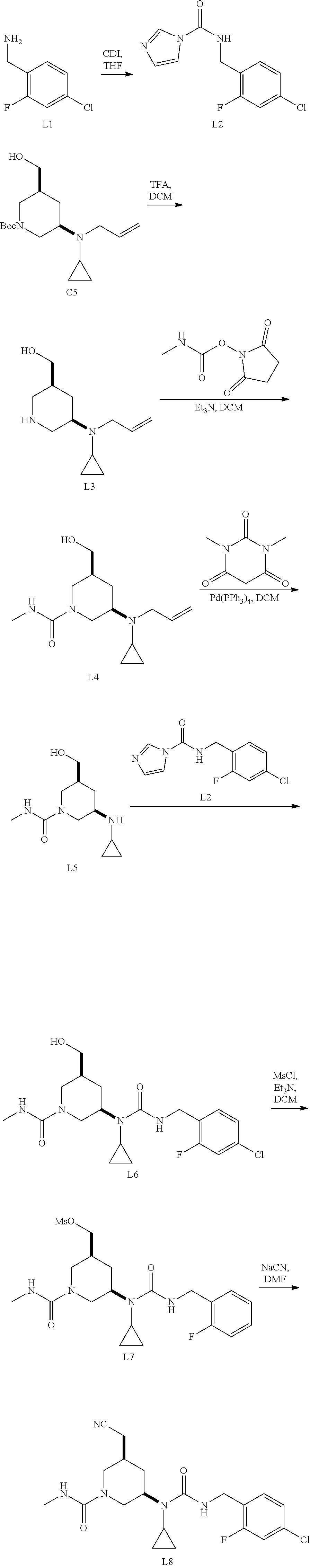

### Step 1: Synthesis of L2

To a solution of L1 (1.59 g, 10 mmol) in THF (100 mL) was added triethylamine (1.01 g, 10 mmol) and CDI (1.78 g, 11 mmol) at 0° C. The resulting mixture was stirred at 0° C. for 1 hr under N₂ atmosphere. After completion, the mixture was diluted with water (80 mL) and extracted with DCM (50 mL×2). The combined organic layers were washed with brine (60 mL), dried over anhydrous Na₂SO₄, filtered and concentrated to dryness under reduced pressure. The residue was purified via flash column chromatography (eluted with 0˜20%/EtOAc in PE) to give L2 (2.6 g, 69% yield) as white solid. LC/MS (ESI) m/z: 254 (M+H)⁺.

### Step 2: Synthesis of L3

To a solution of C5 (1.1 g, 3.5 mmol) in DCM (12 mL) was added TFA (3 mL) dropwise at 0° C. under N₂ atmosphere. The resulting mixture was stirred at room temperature for 5 hrs under N₂ atmosphere. Then the mixture was concentrated under reduced pressure to dryness.

The residue was dissolved in DCM (20 mL), basified with saturated NaHCO₃Solution until the pH was adjusted to pH=8, then the mixture was diluted with water (50 mL) and extracted with DCM (40 mL×2). The combined organic layers were washed with brine (70 mL), dried over anhydrous Na₂SO₄, filtered and concentrated to dryness under reduced pressure. The residue was purified via flash column chromatography (eluted with 20˜30% MeOH in DCM) to give L3 (0.72 g, 96% yield) as light-yellow oil. LC/MS (ESI) m/z: 211 (M+H)⁺.

### Step 3: Synthesis of L4

To a mixture of L3 (720 mg, 3.4 mmol) and TEA (1.0 g, 10.3 mmol) in dry DCM (30 mL) was added 2,5-dioxopyrrolidin-1-yl methylcarbamate (619 mg, 3.6 mmol) dropwise at 0° C. under N₂ atmosphere. The resulting mixture was stirred at room temperature for 12 hrs. Then the mixture was diluted with water (60 mL) and extracted with DCM (40 mL×2). The combined organic layers were washed with brine (50 mL), dried over anhydrous Na₂SO₄, filtered and concentrated under reduced pressure to dryness. The residue was purified via flash column chromatography (eluted with 10˜15% MeOH in DCM) to give L4 (670 mg, 73% yield) as colorless oil. LC/MS (ESI) m/z: 268 (M+H)⁺. L4 (670 mg, 2.5 mmol) was further separated via SFC ((R,R)-WHELK, 250×21.2 mm I.D., 5 μmA, A for CO₂ and B for IPA (0.1% 7 mol/L NH₃ in MeOH), 40 mL/min) to give L4-P1 (143 mg, 21% yield, e.e. 99%) and L4-P2 (164 mg, 25% yield, e.e. 99%) as colorless oil.

### Step 4: Synthesis of L5

To a solution of L4-P2 (50 mg, 0.19 mmol) in DCM (8 mL) were added 1,3-dimethylpyrimidine-2,4,6(1H,3H,5H)-trione (45 mg, 0.29 mmol) and Pd(PPh₃)₄ (23 mg, 0.02 mmol) at 0° C. under N₂ atmosphere. The resulting mixture was stirred at room temperature for 30 mins. Then the mixture was diluted with water (30 mL) and extracted with DCM (20 mL×2). The combined organic layers were washed with brine (30 mL), dried over anhydrous Na₂SO₄, filtered and concentrated to dryness under reduced pressure. The residue was purified via flash column chromatography (eluted with 25˜30% MeOH in DCM) to give L5 (25 mg, 59% yield) as orange oil. LC/MS (ESI) m/z: 228 (M+H)⁺.

### Step 5: Synthesis of L6

To a solution of L5 (25 mg, 0.11 mmol) in anhydrous THF (6 mL) was added TEA (33 mg, 0.33 mmol) and L2 (28 mg, 0.11 mmol) at 0° C. The resulting mixture was stirred at 50° C. for 18 hrs under N₂ atmosphere. Then the mixture was quenched with water (30 mL) and extracted with DCM (20 mL×2). The combined organic layers were washed with brine (30 mL), dried over anhydrous Na₂SO₄, filtered and concentrated to dryness under reduced pressure. The residue was purified via flash column chromatography (eluted with 0˜6% MeOH in DCM) to give L6 (29 mg, 64% yield) as colorless oil. LC/MS (ESI) m/z: 413 (M+H)⁺.

### Step 6: Synthesis of L7

To a solution of L6 (29 mg, 0.07 mmol) in anhydrous DCM (4 mL) was added TEA (21 mg, 0.21 mmol) and MsCl (9 mg, 0.08 mmol) at 0° C., the resulting mixture was stirred at 0° C. for 15 min under N₂ atmosphere. Then the mixture was diluted with H₂O (30 mL) and extracted with EtOAc (20 mL×2). The combined organic layers were washed with brine (30 mL), dried over anhydrous Na₂SO₄, filtered and concentrated under reduced pressure to give crude L7 (30 mg, quant.) as light-yellow oil which was used in the next step directly without further purification.

### Step 7: Synthesis of Example 11/L8

To a solution of L7 (30 mg, 0.06 mmol) in DMF (4 mL) was added NaCN (4 mg, 0.09 mmol). The resulting mixture was stirred at 60° C. for 16 hrs under N₂ atmosphere. Then the mixture was quenched with saturated NH₄Cl solution (50 mL) and extracted with EtOAc (20 mL×2). The combined organic layers were washed with brine (30 mL), dried over anhydrous Na₂SO₄, filtered and concentrated to dryness under reduced pressure. The residue was purified via prep-HPLC to give Example 11/L8 (11 mg, 42% yield) as white solid. LC/MS (ESI) m/z: 422 (M+H)⁺. ¹H NMR (400 MHz, MeOD) δ 7.32 (t, J=8.3 Hz, 1H), 7.20-7.12 (m, 2H), 6.94 (t, J=5.8 Hz, 1H), 4.40 (d, J=5.5 Hz, 2H), 4.14-4.06 (m, 1H), 3.89-3.79 (m, 1H), 3.73-3.60 (m, 1H), 3.18-3.08 (m, 1H), 2.70 (s, 3H), 2.58-2.52 (m, 1H), 2.52-2.39 (m, 3H), 2.07-1.85 (m, 3H), 1.00-0.88 (m, 2H), 0.80-0.69 (m, 2H). ¹⁹F NMR (376 MHz, MeOD) δ −118.40 (s).

TABLE 2
Compounds prepared according to the methods described above.

| | | Observed | |
|---|---|---|---|
| Example | Structure | LCMS M/Z | 1H NMR |
| Example 1 | 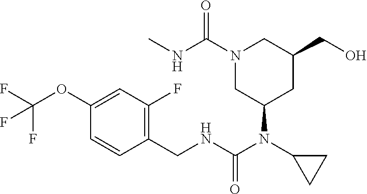 [SMILES: CNC(=O)N1C[C@@H](CO)C[C@@H](N(C(=O)NCc2ccc(OC(F)(F)F)cc2F)C2CC2)C1] (3R,5S)-3-(1-cyclopropyl-3-(2-fluoro- 4-(trifluoromethoxy)benzyl)ureido)-5- (hydroxymethyl)-N-methylpiperidine-1-carboxamide | 463.2 | ¹H NMR (400 MHz, MeOD) δ 7.43 (t, J = 8.6 Hz, 1H), 7.09 (t, J = 7.8 Hz, 2H), 4.50-4.37 (m, 2H), 4.07 (d, J = 9.9 Hz, 1H), 3.88 (d, J = 12.4 Hz, 1H), 3.74-3.61 (m, 1H), 3.49-3.39 (m, 2H), 3.08 (s, 1H), 2.70 (s, 3H), 2.59-2.49 (m, 1H), 2.44- 2.34 (m, 1H), 1.84 (dd, J = 23.5, 11.6 Hz, 2H), 1.77-1.64 (m, 1H), 0.94 (dd, J = 6.2, 3.7 Hz, 2H), 0.76 (d, J = 3.1 Hz, 2H). |
| Example 2 | 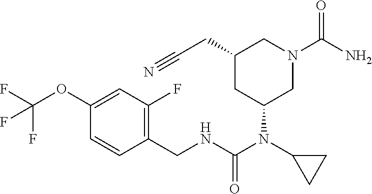 [SMILES: N#CC[C@H]1C[C@@H](N(C(=O)NCc2ccc(OC(F)(F)F)cc2F)C2CC2)CN(C(N)=O)C1] (3R,5R)-3-(cyanomethyl)-5-(1-cyclopropyl-3-(2-fluoro-4- (trifluoromethoxy)benzyl)ureido)piperidine-1-carboxamide | 458.1 | ¹H NMR (400 MHz, MeOD) δ 7.43 (t, J = 8.6 Hz, 1H), 7.09 (t, J = 7.6 Hz, 2H), 6.98 (t, J = 5.9 Hz, 1H), 4.44 (d, J = 5.4 Hz, 2H), 4.11 (d, J = 11.4 Hz, 1H), 3.88 (d, J = 12.7 Hz, 1H), 3.70 (ddd, J = 15.5, 11.1, 4.3 Hz, 1H), 3.25-3.17 (m, 1H), 2.59-2.54 (m, 1H), 2.53-2.41 (m, 3H), 2.02 (dd, J = 18.2, 7.7 Hz, 2H), 1.92 (dd, J = 10.0, 5.4 Hz, 1H), 1.00-0.92 (m, 2H), 0.81-0.73 (m, 2H). |
| Example 3 | 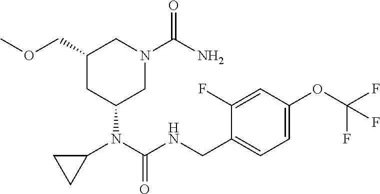 [SMILES: COC[C@H]1C[C@@H](N(C(=O)NCc2ccc(OC(F)(F)F)cc2F)C2CC2)CN(C(N)=O)C1] (3R,5S)-3-(1-cyclopropyl-3-(2-fluoro-4-(trifluoromethoxy)benzyl)ureido)-5-(methoxymethyl)piperidine-1- carboxamide | 463.2 | ¹H NMR (400 MHz,CD3OD) δ 7.44 (t, J = 8.6 Hz, 1H), 7.09 (t, J = 7.7 Hz, 2H), 4.44 (s, 2H), 4.07 (d, J = 12.3 Hz, 1H), 3.91 (d, J = 11.6 Hz, 1H), 3.68 (d, J = 4.1 Hz, 1H), 3.33 (s, 3H), 3.28 (d, J = 6.2 Hz, 2H), 3.21-3.12 (m, 1H), 2.59-2.51 (m, 1H), 2.49- 2.39 (m, 1H), 1.89 (d, J = 7.2 Hz, 3H), 0.97-0.91 (m, 2H), 0.81-0.70 (m, 2H). |
| Example 4 | 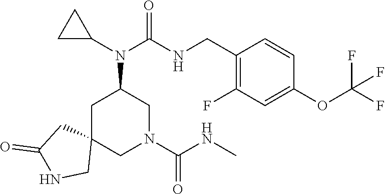 [SMILES: CNC(=O)N1C[C@H](N(C(=O)NCc2ccc(OC(F)(F)F)cc2F)C2CC2)C[C@]2(CNC(=O)C2)C1] (5S,9R)-9-(1-cyclopropyl-3-(2-fluoro-4- (trifluoromethoxy)benzyl)ureido)-N-methyl-3-oxo-2,7- diazaspiro[ 4.5] decane-7-carboxamide | 502.2 | ¹H NMR (400 MHz, MeOD) δ 7.49- 7.41 (m, 1H), 7.16-7.07 (m, 2H), 4.51-4.39 (m, 2H), 4.09- 4.02 (m, 1H), 3.97-3.88 (m, 1H), 3.80-3.67 (m, 1H), 3.22-3.14 (m, 3H), 2.72 (s, 3H), 2.66-2.60 (m, 1H), 2.59-2.53 (m, 1H), 2.38-2.31 (m, 1H), 2.30-2.21 (m, 2H), 1.94- 1.86 (m, 1H), 1.02-0.95 (m, 2H), 0.82-0.75 (m, 2H). 19F NMR (377 MHz, MeOD) δ −59.77 (s), −116.97 (s). |
| Example 5 | 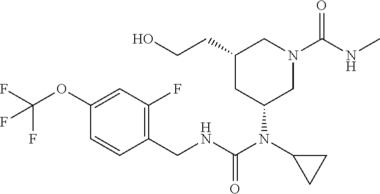 [SMILES: CNC(=O)N1C[C@@H](CCO)C[C@@H](N(C(=O)NCc2ccc(OC(F)(F)F)cc2F)C2CC2)C1] (3R,5R)-3-(1-cyclopropyl-3-(2-fluoro-4- (trifluoromethoxy)benzyl)ureido)-5-(2-hydroxyethyl)- N-methylpiperidine-1-carboxamide | 477.3 | 1HNMR(400MHz,MeOD) δ7.43(t, J = 8.6Hz,1H), 7.09(t, J = 7.7 Hz, 2H), 6.95(t, J = 5.9 Hz,1H),4.44(d, J = 4.7Hz, 2H), 4.02-3.94(m, 1H), 3.91- 3.82 (m, 1H), 3.69-3.58 (m, 3H), 3.16-3.08 (m, 1H), 2.69 (d, J = 3.9 Hz, 3H), 2.58-2.50 (m, 1H), 2.33 (dd, J = 13.1, 11.4 Hz, 1H), 1.95 (d, J = 12.1 Hz, 1H), 1.79 (q, J = 11.9 Hz, 1H), 1.67 (ddd, J = 10.7, 7.5, 3.8 Hz, 1H), 1.58-1.37 (m, 2H), 1.00-0.88 (m, 2H), 0.81-0.70 (m, 2H). |
| Example 6 | 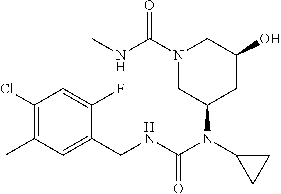 [SMILES: CNC(=O)N1C[C@@H](O)C[C@@H](N(C(=O)NCc2cc(C)c(Cl)cc2F)C2CC2)C1] (3R,5S)-3-(3-(4-chloro-2-fluoro-5-methylbenzyl)-1- cyclopropylureido)-5-hydroxy-N-methylpiperidine-1-carboxamide | 413.2 | ¹H NMR (400 MHz, MeOD) δ 7.24 (d, J = 8.0 Hz, 1H), 7.13 (d, J = 9.8 Hz, 1H), 6.90 (t, J = 5.9 Hz, 1H), 4.42- 4.31 (m, 2H), 4.14 (dd, J = 12.7, 4.8 Hz, 1H), 3.78 (dd, J = 12.7, 4.1 Hz, 1H), 3.67 (tt, J = 12.1, 3.9 Hz, 1H), 3.58-3.48 (m, 1H), 3.11-3.02 (m, 1H), 2.69 (s, 3H), 2.55- 2.48 (m, 1H), 2.41 (dd, J = 12.7, 10.8 Hz, 1H), 2.32 (s, 3H), 2.14 (d, J = 11.4 Hz, 1H), 2.02 (dd, J = 23.2, 11.7 Hz, 1H), 0.99-0.90 (m, 2H), 0.80-0.70 (m, 2H). |
| Example 7 | 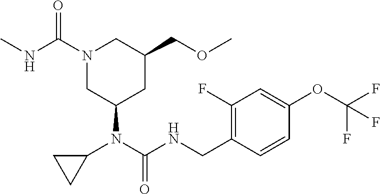 [SMILES: CNC(=O)N1C[C@@H](COC)C[C@@H](N(C(=O)NCc2ccc(OC(F)(F)F)cc2F)C2CC2)C1] (3R,5S)-3-(1-cyclopropyl-3-(2-fluoro-4- (trifluoromethoxy)benzyl)ureido)-5- (methoxymethyl)-N-methylpiperidine-1-carboxamide | 477.3 | ¹H NMR (400 MHz, MeOD) δ 7.43 (t, J = 8.6 Hz, 1H), 7.09 (t, J = 7.8 Hz, 2H), 4.43 (s, 2H), 4.05 (d, J = 14.0 Hz, 1H), 3.88 (dd, J = 12.5, 4.0 Hz, 1H), 3.74-3.58 (m, 1H), 3.32 (s, 3H), 3.28 (t, J = 5.5 Hz, 2H), 3.16-3.02 (m, 1H), 2.70 (s, 3H), 2.53 (td, J = 6.6, 3.4 Hz, 1H), 2.40 (dd, J = 13.3, 10.8 Hz, 1H), 1.86 (d, J = 8.5 Hz, 3H), 0.97-0.91 (m, 2H), 0.79- 0.71 (m, 2H). |
| Example 8 | 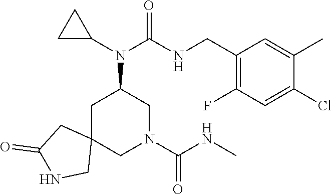 [SMILES: CNC(=O)N1C[C@H](N(C(=O)NCc2cc(C)c(Cl)cc2F)C2CC2)CC2(CNC(=O)C2)C1] (9R)-9-(3-(4-chloro-2-fluoro-5-methylbenzyl)-1- cyclopropylureido)-N-methyl-3-oxo-2,7- diazaspiro[4.5]decane-7-carboxamide | 466.2 | ¹H NMR (400 MHz, MeOD) δ 7.26 (d, J = 8.0 Hz, 1H), 7.15 (d, J = 9.8 Hz, 1H), 6.98-6.88 (m, 1H), 4.46-4.33 (m, 2H), 4.12-4.01 (m, 1H), 3.96- 3.86 (m, 1H), 3.82-3.68 (m, 1H), 3.32-3.30 (m, 1H), 3.24-3.18 (m, 1H), 3.18-3.15 (m, 1H), 3.15-3.09 (m, 1H), 2.72 (d, J = 0.9 Hz, 3H), 2.66-2.57 (m, 1H), 2.57-2.48 (m, 1H), 2.39-2.34 (m, 1H), 2.33 (s, 3H), 2.31-2.21 (m, 2H), 2.17-2.10 (m, 1H), 2.00-1.86 (m, 1H), 1.01-0.91 (m, 2H), 0.82- 0.72 (m, 2H). |
| Example 9 | 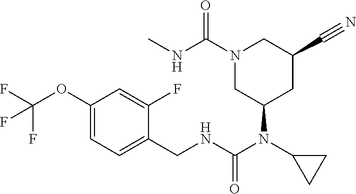 [SMILES: CNC(=O)N1C[C@@H](C#N)C[C@@H](N(C(=O)NCc2ccc(OC(F)(F)F)cc2F)C2CC2)C1] (3S,5R)-3-cyano-5-(1-cyclopropyl-3-(2-fluoro-4- (trifluoromethoxy)benzyl)ureido)-N-methylpiperidine-1- carboxamide | 458.2 | ¹H NMR (400 MHz, CD3OD) δ 7.43 (t, J = 8.6 Hz, 1H), 7.10 (t, J = 7.7 Hz, 2H), 4.44 (s, 2H), 4.29 (d, J = 9.6 Hz, 1H), 3.85 (d, J = 12.1 Hz, 1H), 3.63- 3.52 (m, 1H), 3.26 (s, 1H), 2.90-2.79 (m, 2H), 2.71 (s, 3H), 2.62-2.54 (m, 1H), 2.48-2.37 (m, 1H), 2.26 (d, J = 12.6 Hz, 1H), 1.03-0.92 (m, 2H), 0.78 (s, 2H). |
| Example 10 | 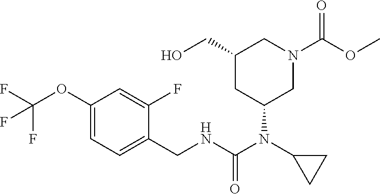 [SMILES: COC(=O)N1C[C@@H](CO)C[C@@H](N(C(=O)NCc2ccc(OC(F)(F)F)cc2F)C2CC2)C1] methyl (3R,5S)-3-(1-cyclopropyl-3-(2-fluoro-4- (trifluoromethoxy)benzyl)ureido)-5- (hydroxymethyl)piperidine-1-carboxylate | 464.1 | ¹H NMR (400 MHz, MeOD) δ 7.42 (t, J = 8.6 Hz, 1H), 7.09 (t, J = 8.4 Hz, 2H), 4.43 (s, 2H), 4.20 (d, J = 11.7 Hz, 1H), 4.03 (s, 1H), 3.68 (s, 4H), 3.54- 3.36 (m, 2H), 3.16 (dd, J = 20.0, 7.7 Hz, 1H), 2.48 (d, J = 45.0 Hz, 2H), 1.85 (t, J = 9.9 Hz, 2H), 1.73 (d, J = 8.8 Hz, 1H), 0.94 (dd, J = 6.5, 2.4 Hz, 2H), 0.75 (s, 2H). |
| Example 11 | 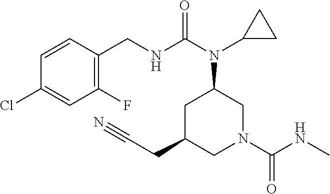 [SMILES: CNC(=O)N1C[C@@H](CC#N)C[C@@H](N(C(=O)NCc2ccc(Cl)cc2F)C2CC2)C1] (3R,5R)-3-(3-(4-chloro-2- fluorobenzyl)-1-cyclopropylureido)-5- (cyanomethyl)-N-methylpiperidine-1-carboxamide | 422.2 | ¹H NMR (400 MHz, MeOD) δ 7.32 (t, J = 8.3 Hz, 1H), 7.20-7.12 (m, 2H), 6.94 (t, J = 5.8 Hz, 1H), 4.40 (d, J = 5.5 Hz, 2H), 4.14-4.06 (m, 1H), 3.89-3.79 (m, 1H), 3.73- 3.60 (m, 1H), 3.13 (s, 1H), 2.70 (s, 3H), 2.58-2.52 (m, 1H), 2.52-2.39 (m, 3H), 2.07-1.85 (m, 3H), 1.00-0.88 (m, 2H), 0.80- 0.69 (m, 2H). |

TABLE 3
Compounds prepared according to adaptations of the methods used to prepare
Examples 1-11 in Table 1.

| | | Observed | |
|---|---|---|---|
| Example | Structure | LCMS M/Z | 1H NMR |
| Example 12 | 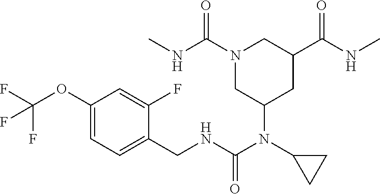 [SMILES: CNC(=O)C1CC(N(C(=O)NCc2ccc(OC(F)(F)F)cc2F)C2CC2)CN(C(=O)NC)C1] | 490.3 | |
| | 5-(1-cyclopropyl-3-(2-fluoro-4- | | |
| | (trifluoromethoxy)benzyl)ureido)- | | |
| | N¹,N³-dimethylpiperidine-1,3- | | |
| | dicarboxamide | | |
| Example 13 | 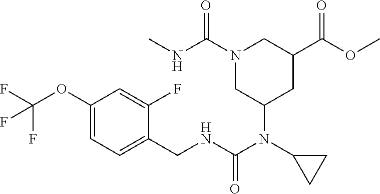 [SMILES: CNC(=O)N1CC(C(=O)OC)CC(N(C(=O)NCc2ccc(OC(F)(F)F)cc2F)C2CC2)C1] | 491.3 | |
| | methyl 5-(1-cyclopropyl-3-(2-fluoro-4- | | |
| | (trifluoromethoxy)benzyl)ureido)-1- | | |
| | (methylcarbamoyl)piperidine-3- | | |
| | carboxylate | | |
| Example 14 | 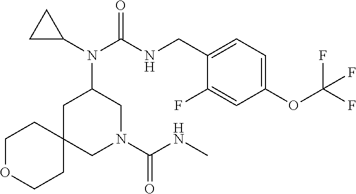 [SMILES: CNC(=O)N1CC(N(C(=O)NCc2ccc(OC(F)(F)F)cc2F)C2CC2)CC2(CCOCC2)C1] | 503.3 | ¹H NMR (400 MHz, dmso) δ 7.48 (t, J = 8.5 Hz, 1H), 7.40-7.35 (m, 1H), 7.32- 7.25 (m, 1H), 6.98 (t, J = 5.9 Hz, 1H), 6.40 (q, J = 4.3 Hz, 1H), 4.36 (d, J = 5.8 Hz, 2H), 4.17 (d, J = 13.4 Hz, 1H), 4.02-3.92 (m, 1H), 3.75 (ddd, J = 12.1, 8.5, 2.9 Hz, 1H), 3.67- 3.51 (m, 4H), 3.02 (t, J = 11.9 Hz, 1H), 2.59 (d, J = |
| | 4-(1-cyclopropyl-3-(2-fluoro-4- | | 4.2 Hz, 3H), 2.56 (q, J = |
| | (trifluoromethoxy)benzyl)ureido)-N- | | 1.9 Hz, 1H), 2.33 (s, 1H), |
| | methyl-9-oxa-2- | | 1.83 (t, J = 12.5 Hz, 1H), |
| | azaspiro[5.5]undecane-2-carboxamide | | 1.72 (d, J = 12.4 Hz, 1H), |
| | | | 1.48 (dt, J = 9.1, 4.4 Hz, |
| | | | 1H), 1.43-1.25 (m, 3H), |
| | | | 0.95-0.92 (m, 2H), 0.74- |
| | | | 0.64 (m, 2H) |
| Example 15 | 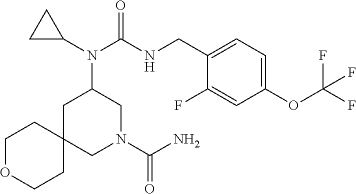 [SMILES: NC(=O)N1CC(N(C(=O)NCc2ccc(OC(F)(F)F)cc2F)C2CC2)CC2(CCOCC2)C1] | 489.3 | ¹H NMR (400 MHz, dmso) δ 7.42 (t, J = 8.5 Hz, 1H), 7.32 (d, J = 10.1 Hz, 1H), 7.22 (d, J = 8.5 Hz, 1H), 6.92 (t, J = 5.9 Hz, 1H), 5.88 (s, 2H), 4.30 (d, J = 5.8 Hz, 2H), 4.10 (d, J = 13.6 Hz, 1H), 3.91 (d, J = 12.2 Hz, 1H), 3.69 (t, J = 12.0 Hz, 1H), 3.61-3.48 (m, 4H), 2.98 (t, J = 11.9 Hz, 1H), 2.48-2.42 (m, |
| | 4-(1-cyclopropyl-3-(2-fluoro-4- | | 1H), 2.28 (d, J = 13.4 Hz, |
| | (trifluoromethoxy)benzyl)ureido)-9- | | 1H), 1.77 (t, J = 12.5 Hz, |
| | oxa-2-azaspiro[5.5]undecane-2- | | 1H), 1.66 (d, J = 12.7 Hz, |
| | carboxamide | | 1H), 1.47 (d, J = 14.3 Hz, |
| | | | 1H), 1.36-1.23 (m, 3H), |
| | | | 0.88-0.87 (m, 2H), 0.65- |
| | | | 0.64 (m, 2H). |
| Example 16 | 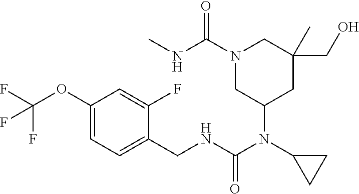 [SMILES: CNC(=O)N1CC(N(C(=O)NCc2ccc(OC(F)(F)F)cc2F)C2CC2)CC(C)(CO)C1] | 477.4 | |
| | 5-(1-cyclopropyl-3-(2-fluoro-4- | | |
| | (trifluoromethoxy)benzyl)ureido)-3- | | |
| | (hydroxymethyl)-N,3- | | |
| | dimethylpiperidine-1-carboxamide | | |
| Example 17 | 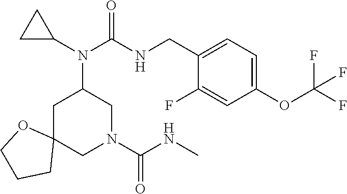 [SMILES: CNC(=O)N1CC(N(C(=O)NCc2ccc(OC(F)(F)F)cc2F)C2CC2)CC2(CCCO2)C1] | 489.2 | |
| | 9-(1-cyclopropyl-3-(2-fluoro-4- | | |
| | (trifluoromethoxy)benzyl)ureido)-N- | | |
| | methyl-1-oxa-7-azaspiro[4.5]decane-7- | | |
| | carboxamide | | |
| Example 18 | 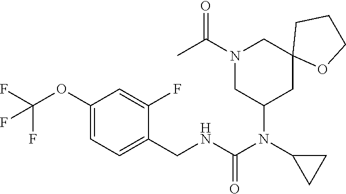 [SMILES: CC(=O)N1CC(N(C(=O)NCc2ccc(OC(F)(F)F)cc2F)C2CC2)CC2(CCCO2)C1] | 474.3 | |
| | 1-(7-acetyl-1-oxa-7- | | |
| | azaspiro[4.5]decan-9-yl)-1- | | |
| | cyclopropyl-3-(2-fluoro-4- | | |
| | (trifluoromethoxy)benzyl)urea | | |
| Example 19 | 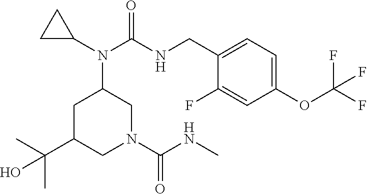 [SMILES: CNC(=O)N1CC(N(C(=O)NCc2ccc(OC(F)(F)F)cc2F)C2CC2)CC(C(C)(C)O)C1] | 491.3 | ¹H NMR (400 MHz, dmso) δ 7.42 (t, J = 8.5 Hz, 1H), 7.33 (dd, J = 10.4, 2.4 Hz, 1H), 7.26-7.19 (m, 1H), 6.94 (dt, J = 11.8, 5.9 Hz, 1H), 6.30 (p, J = 4.7 Hz, 1H), 4.31 (d, J = 5.9 Hz, 2H), 4.21 (s, 1H), 4.02 (d, J = 12.4 Hz, 1H), 3.78 (dd, J = 12.4, 4.1 Hz, 1H), 3.47 (td, J = 11.1, 5.7 Hz, 1H), 2.95 (dt, J = 23.6, 11.9 Hz, |
| | 3-(1-cyclopropyl-3-(2-fluoro-4- | | 1H), 2.53 (d, J = 4.3 Hz, |
| | (trifluoromethoxy)benzyl)ureido)-5- | | 3H), 2.48-2.43 (m, 1H), |
| | (2-hydroxypropan-2-yl)-N- | | 2.28 (t, J = 12.3 Hz, 1H), |
| | methylpiperidine-1-carboxamide | | 1.81-1.68 (m, 2H), 1.37- |
| | | | 1.27 (m, 1H), 1.05 (s, 6H), |
| | | | 0.92-0.80 (m, 2H), 0.67- |
| | | | 0.63 (m, 2H). |
| Example 20 | 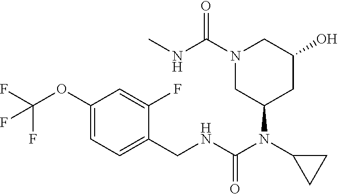 [SMILES: CNC(=O)N1C[C@H](O)C[C@@H](N(C(=O)NCc2ccc(OC(F)(F)F)cc2F)C2CC2)C1] | 449.3 | |
| | (3R,5R)-3-(1-cyclopropyl-3-(2-fluoro- | | |
| | 4-(trifluoromethoxy)benzyl)ureido)-5- | | |
| | hydroxy-N-methylpiperidine-1- | | |
| | carboxamide | | |
| Example 22 | 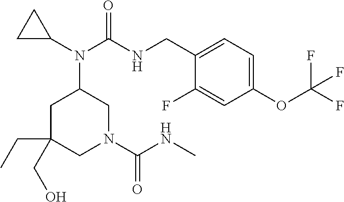 [SMILES: CCC1(CO)CC(N(C(=O)NCc2ccc(OC(F)(F)F)cc2F)C2CC2)CN(C(=O)NC)C1] | 491.4 | |
| | 5-(1-cyclopropyl-3-(2-fluoro-4- | | |
| | (trifluoromethoxy)benzyl)ureido)-3- | | |
| | ethyl-3-(hydroxymethyl)-N- | | |
| | methylpiperidine-1-carboxamide | | |
| Example 25 | 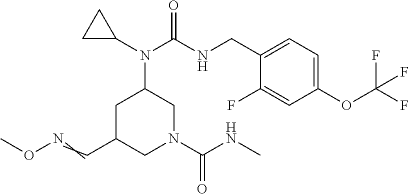 [SMILES: CNC(=O)N1CC(C=NOC)CC(N(C(=O)NCc2ccc(OC(F)(F)F)cc2F)C2CC2)C1] | 490.3 | ¹H NMR (400 MHz, dmso) δ 7.42 (t, J = 8.5 Hz, 1H), 7.40-7.29 (m, 2H), 7.22 (d, J = 8.5 Hz, 1H), 6.95 (t, J = 5.9 Hz, 1H), 6.46 (q, J = 3.9 Hz, 1H), 4.31 (d, J = 5.8 Hz, 2H), 4.00 (d, J = 13.0 Hz, 1H), 3.80 (d, J = 19.6 Hz, 2H), 3.72 (d, J = 0.7 Hz, 2H), 3.54-3.43 (m, 1H), 3.03 (dd, J = 13.5, 10.4 Hz, 1H), 2.54 (dd, J = |
| | 3-(1-cyclopropyl-3-(2-fluoro-4- | | 4.6, 1.8 Hz, 3H), 2.51- |
| | (trifluoromethoxy)benzyl)ureido)-5- | | 2.39 (m, 2H), 2.35-2.23 |
| | ((methoxyimino)methyl)-N- | | (m, 1H), 2.03-1.89 (m, |
| | methylpiperidine-1-carboxamide | | 1H), 1.89-1.74 (m, 1H), |
| | | | 0.92-0.84 (m, 2H), 0.72- |
| | | | 0.62 (m, 2H). |
| Example 27 | 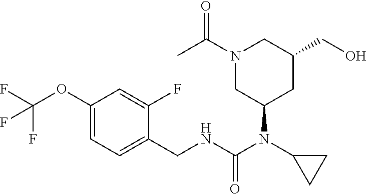 [SMILES: CC(=O)N1C[C@H](CO)C[C@@H](N(C(=O)NCc2ccc(OC(F)(F)F)cc2F)C2CC2)C1] | 448.3 | ¹H NMR (400 MHz, dmso) δ 7.42 (dt, J = 10.7, 8.5 Hz, 1H), 7.33 (dt, J = 10.5, 3.0 Hz, 1H), 7.22 (d, J = 8.6 Hz, 1H), 6.98-6.90 (m, 1H), 4.72 (s, 1H), 4.40- 4.24 (m, 3H), 3.83-3.73 (m, 1H), 3.66-3.50 (m, 1H), 3.32-3.14 (m, 1H), 3.03 (dd, J = 13.6, 3.4 Hz, 1H), 2.83 (t, J = 11.7 Hz, 1H), 2.42 (tq, J = 6.3, |
| | 1-((3R,5R)-1-acetyl-5- | | 3.1 Hz, 1H), 2.24-2.06 |
| | (hydroxymethyl)piperidin-3-yl)-1- | | (m, 1H), 1.97 (s, 3H), 1.92- |
| | cyclopropyl-3-(2-fluoro-4- | | 1.83 (m, 2H), 1.64 (d, J = |
| | (trifluoromethoxy)benzyl)urea | | 12.8 Hz, 1H), 0.92-0.82 |
| | | | (m, 2H), 0.67-0.57 (m, |
| | | | 2H). |
| Example 21 | 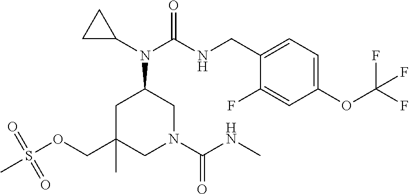 [SMILES: CNC(=O)N1C[C@H](N(C(=O)NCc2ccc(OC(F)(F)F)cc2F)C2CC2)CC(C)(COS(C)(=O)=O)C1] | 555.2 | ¹H NMR (400 MHz, dmso) δ 7.42 (t, J = 8.5 Hz, 1H), 7.33 (dd, J = 10.3, 2.5 Hz, 1H), 7.23 (dd, J = 8., 2.3 Hz, 1H), 6.95 (t, J = 5.9 Hz, 1H), 6.38 (q, J = 4.3 Hz, 1H), 4.34-4.28 (m, 2H), 3.99 (s, 2H), 3.85 (d, J = 13.2 Hz, 2H), 3.62 (s, 1H), 3.16 (s, 3H), 3.02 (t, J = 11.8 Hz, 1H), 2.62-2.52 (m, 3H), 2.46 (dq, J = 6.8, |
| | ((5R)-5-(1-cyclopropyl-3-(2-fluoro-4- | | 3.4 Hz, 1H), 2.38 (d, J = |
| | (trifluoromethoxy)benzyl)ureido)-3- | | 13.7 Hz, 1H), 2.02-1.84 |
| | methyl-1- | | (m, 1H), 1.70 (d, J = |
| | (methylcarbamoyl)piperidin-3- | | 12.7 Hz, 1H), 0.92 (s, 3H), |
| | yl)methyl methanesulfonate | | 0.96-0.80 (m, 2H), 0.70- |
| | | | 0.61 (m, 2H). |
| Example 29 | 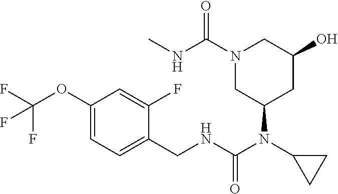 [SMILES: CNC(=O)N1C[C@@H](O)C[C@@H](N(C(=O)NCc2ccc(OC(F)(F)F)cc2F)C2CC2)C1] | 449.3 | ¹H NMR (400 MHz, dmso) δ 7.48 (t, J = 8.5 Hz, 1H), 7.38 (dd, J = 10.4, 2.4 Hz, 1H), 7.32-7.24 (m, 1H), 6.99 (t, J = 5.9 Hz, 1H), 6.46 (q, J = 4.3 Hz, 1H), 4.98 (s, br, 1H) 4.37 (d, J = 5.8 Hz, 2H), 4.05 (dd, J = 12.6, 4.7 Hz, 1H), 3.81 (dd, J = 12.4, 4.2 Hz, 1H), 3.56 (tt, J = 11.7, 4.0 Hz, 1H), 3.45-3.33 (m, 2H), 2.93 |
| | (3R,5S)-3-(1-cyclopropyl-3-(2-fluoro- | | (t, J = 11.8 Hz, 1H), 2.59 |
| | 4-(trifluoromethoxy)benzyl)ureido)-5- | | (d, J = 4.2 Hz, 3H), 2.27 |
| | hydroxy-N-methylpiperidine-1- | | (dd, J = 12.6, 10.5 Hz, 1H), |
| | carboxamide | | 2.05-1.96 (m, 1H), 1.97- |
| | | | 1.83 (m, 1H), 1.00-0.87 |
| | | | (m, 2H), 0.79-0.65 (m, |
| | | | 2H). |
| Example 30 | 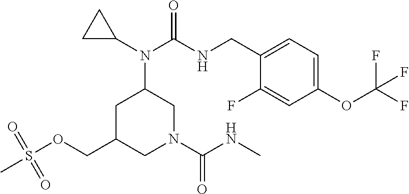 [SMILES: CNC(=O)N1CC(COS(C)(=O)=O)CC(N(C(=O)NCc2ccc(OC(F)(F)F)cc2F)C2CC2)C1] | 541.2 | |
| | (5-(1-cyclopropyl-3-(2-fluoro-4- | | |
| | (trifluoromethoxy)benzyl)ureido)-1- | | |
| | (methylcarbamoyl)piperidin-3- | | |
| | yl)methyl methanesulfonate | | |
| Example 31 | 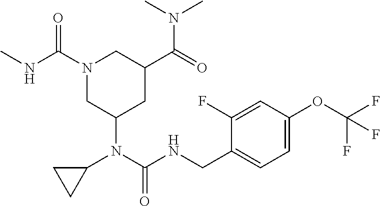 [SMILES: CNC(=O)N1CC(C(=O)N(C)C)CC(N(C(=O)NCc2ccc(OC(F)(F)F)cc2F)C2CC2)C1] | 504.3 | ¹H NMR (400 MHz, dmso) δ 7.46 (t, J = 8.5 Hz, 1H), 7.37 (dd, J = 10.4, 2.4 Hz, 1H), 7.31-7.22 (m, 1H), 6.97 (t, J = 5.9 Hz, 1H), 6.49 (q, J = 4.3 Hz, 1H), 4.35 (d, J = 5.8 Hz, 2H), 3.93 (d, J = 12.9 Hz, 1H), 3.87 (dd, J = 12.7, 4.1 Hz, 1H), 3.61 (tt, J = 11.9, 4.1 Hz, 1H), 3.06 (s, 3H), 2.84 (s, 3H), 2.78-2.69 (m, 1H), 2.66-2.61 (m, 1H), |
| | 5-(1-cyclopropyl-3-(2-fluoro-4- | | 2.59 (d, J = 4.1 Hz, 3H), |
| | (trifluoromethoxy)benzyl)ureido)- | | 2.50 (dq, J = 6.9, 3.5 Hz, |
| | N¹,N³,N³-trimethylpiperidine-1,3- | | 1H), 2.15 (q, J = 12.2 Hz, |
| | dicarboxamide | | 1H), 1.82 (d, J = 12.0 Hz, |
| | | | 1H), 0.96-0.87 (m, 2H), |
| | | | 0.76-0.64 (m, 2H). |
| Example 28 | 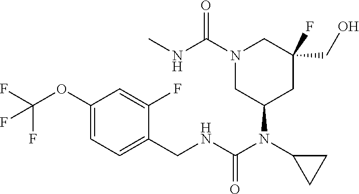 [SMILES: CNC(=O)N1C[C@H](N(C(=O)NCc2ccc(OC(F)(F)F)cc2F)C2CC2)C[C@@](F)(CO)C1] | 481.3 | ¹H NMR (400 MHz, dmso) δ 7.46 (t, J = 8.5 Hz, 1H), 7.36 (dd, J = 10.3, 2.4 Hz, 1H), 7.29-7.22 (m, 1H), 7.02 (t, J = 5.9 Hz, 1H), 6.48 (q, J = 4.3 Hz, 1H), 5.10 (s, 1H), 4.42-4.27 (m, 2H), 4.05 (d, J = 13.0 Hz, 1H), 3.87-3.79 (m, 1H), 3.61-3.48 (m, 2H), 3.44 (d, J = 13.6 Hz, 1H), 3.08 (t, J = 11.9 Hz, 1H), |
| | (3S,5R)-5-(1-cyclopropyl-3-(2-fluoro- | | 2.71 (dd, J = 13.0, 6.8 Hz, |
| | 4-(trifluoromethoxy)benzyl)ureido)-3- | | 1H), 2.58 (d, J = 4.2 Hz, |
| | fluoro-3-(hydroxymethyl)-N- | | 3H), 2.53-2.45 (m, 1H), |
| | methylpiperidine-1-carboxamide | | 2.28-2.14 (m, 2H), 0.99- |
| | | | 0.86 (m, 2H), 0.80-0.62 |
| | | | (m, 2H). |
| Example 36 | 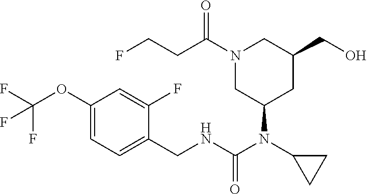 [SMILES: O=C(CCF)N1C[C@@H](CO)C[C@@H](N(C(=O)NCc2ccc(OC(F)(F)F)cc2F)C2CC2)C1] | 480.1 | ¹H NMR (400 MHz, dmso) δ 7.48-7.37 (m, 1H), 7.33 (dt, J = 10.4, 2.8 Hz, 1H), 7.26-7.19 (m, 1H), 7.00- 6.89 (m, 1H), 4.70 (q, J = 5.5 Hz, 1H), 4.59 (q, J = 5.6 Hz, 1H), 4.54-4.46 (m, 1H), 4.32 (t, J = 6.1 Hz, 2H), 3.87 (d, J = 12.4 Hz, 1H), 3.73 (d, J = 13.0 Hz, 1H), 3.65-3.43 (m, 1H), 3.38-3.21 (m, 2H), |
| | 1-cyclopropyl-3-(2-fluoro-4- | | 3.16 (t, J = 12.1 Hz, 1H), |
| | (trifluoromethoxy)benzyl)-1-((3R,5S)- | | 2.89-2.67 (m, 2H), 2.64- |
| | 1-(3-fluoropropanoyl)-5- | | 2.51 (m, 1H), 2.50-2.41 |
| | (hydroxymethyl)piperidin-3-yl)urea | | (m, 1H), 2.11 (t, J = |
| | | | 12.2 Hz, 1H), 1.81-1.71 |
| | | | (m, 2H), 0.93-0.80 (m, |
| | | | 2H), 0.73-0.59 (m, 2H). |
| Example 37 | 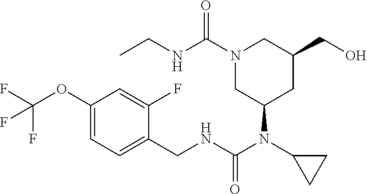 [SMILES: CCNC(=O)N1C[C@@H](CO)C[C@@H](N(C(=O)NCc2ccc(OC(F)(F)F)cc2F)C2CC2)C1] | 477.3 | ¹H NMR (400 MHz, dmso) δ 7.42 (t, J = 8.5 Hz, 1H), 7.33 (dd, J = 10.3, 2.4 Hz, 1H), 7.26-7.19 (m, 1H), 6.93 (t, J = 5.9 Hz, 1H), 6.39 (t, J = 5.4 Hz, 1H), 4.51 (s, 1H), 4.31 (d, J = 5.8 Hz, 2H), 4.00 (dd, J = 12.7, 3.9 Hz, 1H), 3.82 (dd, J = 12.4, 4.0 Hz, 1H), 3.51 (tt, J = 11.5, 4.1 Hz, 1H), 3.26 (t, J = 5.6 Hz, 2H), |
| | (3R,5S)-3-(1-cyclopropyl-3-(2-fluoro- | | 3.07-2.96 (m, 2H), 2.91 |
| | 4-(trifluoromethoxy)benzyl)ureido)-N- | | (t, J = 11.8 Hz, 1H), 2.45 |
| | ethyl-5-(hydroxymethyl)piperidine-1- | | (tt, J = 6.8, 3.8 Hz, 1H), |
| | carboxamide | | 2.22-2.12 (m, 1H), 1.77- |
| | | | 1.70 (m, 1H), 1.64 (q, J = |
| | | | 11.8 Hz, 1H), 1.54-1.49 |
| | | | (m, 1H), 0.98 (t, J = 7.1 Hz, |
| | | | 3H), 0.92-0.83 (m, |
| | | | 2H), 0.72-0.61 (m, 2H). |
| Example 34 | 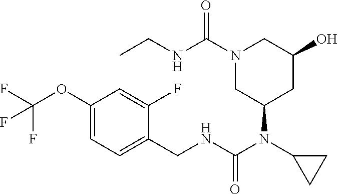 [SMILES: CCNC(=O)N1C[C@@H](O)C[C@@H](N(C(=O)NCc2ccc(OC(F)(F)F)cc2F)C2CC2)C1] | 463.3 | ¹H NMR (400 MHz, dmso) δ 7.42 (t, J = 8.5 Hz, 1H), 7.37-7.29 (m, 1H), 7.26- 7.19 (m, 1H), 6.94 (t, J = 5.9 Hz, 1H), 6.45 (t, J = 5.4 Hz, 1H), 4.92 (d, J = 4.8 Hz, 1H), 4.31 (d, J = 5.8 Hz, 2H), 4.00 (dd, J = 12.6, 4.7 Hz, 1H), 3.76 (dd, J = 12.6, 4.1 Hz, 1H), 3.50 (tt, J = 11.8, 4.0 Hz, 1H), 3.36 (s, br, 1H) 3.06-2.95 |
| | (3R,5S)-3-(1-cyclopropyl-3-(2-fluoro- | | (m, 2H), 2.92-2.79 (m, 1H), |
| | 4-(trifluoromethoxy)benzyl)ureido)-N- | | 2.44 (tt, J = 6.9, 3.8 Hz, |
| | ethyl-5-hydroxypiperidine-1- | | 1H), 2.20 (dd, J = 12.5, |
| | carboxamide | | 10.6 Hz, 1H), 1.99-1.90 |
| | | | (m, 1H), 1.84 (q, J = |
| | | | 11.6 Hz, 1H), 0.98 (t, J = |
| | | | 7.1 Hz, 3H), 0.94-0.82 (m, |
| | | | 2H), 0.71-0.59 (m, 1H). |
| Example 35 | 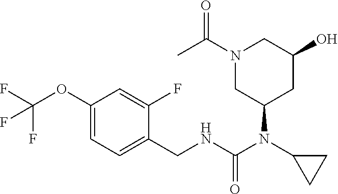 [SMILES: CC(=O)N1C[C@@H](O)C[C@@H](N(C(=O)NCc2ccc(OC(F)(F)F)cc2F)C2CC2)C1] | 434.3 | ¹H NMR (400 MHz, dmso) δ 7.43 (q, J = 8.2 Hz, 1H), 7.37-7.29 (m, 1H), 7.22 (d, J = 8.6 Hz, 1H), 6.95 (dt, J = 18.0, 5.9 Hz, 1H), 5.06 (s, 1H), 4.41 (dd, J = 12.2, 4.8 Hz, 1H), 4.40- 4.28 (m, 2H), 4.23 (dd, J = 11.8, 4.3 Hz, 1H), 3.75 (dd, J = 12.8, 4.8 Hz, 1H), 3.67- 3.54 (m, 1H), 3.50-3.42 (m, 1H), 3.09 (dd, J = 13.8, |
| | 1-((3R,5S)-1-acetyl-5- | | 12.3 Hz, 1H), 2.74 (t, J = |
| | hydroxypiperidin-3-yl)-1-cyclopropyl- | | 11.7 Hz, 1H), 2.61 (dd, J = |
| | 3-(2-fluoro-4- | | 12.9, 10.6 Hz, 1H), 2.44 |
| | (trifluoromethoxy)benzyl)urea | | (tq, J = 7.1, 3.6 Hz, 1H), |
| | | | 2.12 (t, J = 11.4 Hz, 1H), |
| | | | 1.96 (s, 3H), 0.94-0.84 |
| | | | (m, 2H), 0.77-0.56 (m, |
| | | | 2H). |
| Example 33 | 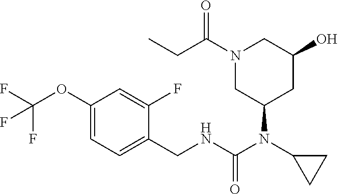 [SMILES: CCC(=O)N1C[C@@H](O)C[C@@H](N(C(=O)NCc2ccc(OC(F)(F)F)cc2F)C2CC2)C1] | 448.3 | ¹H NMR (400 MHz, dmso) δ 7.48-7.37 (m, 1H), 7.33 (dd, J = 10.4, 2.6 Hz, 1H), 7.22 (d, J = 8.5 Hz, 1H), 7.01-6.89 (m, 1H), 4.44 (dd, J = 12.1, 4.7 Hz, 1H), 4.40-4.21 (m, 3H), 3.79 (dd, J = 12.7, 4.6 Hz, 1H), 3.68-3.54 (m, 2H), 3.51- 3.37 (m, 1H), 3.30 (dt, J = 10.2, 4.8 Hz, 1H), 3.04 (t, J = 11.9 Hz, 1H), 2.83-2.69 |
| | 1-cyclopropyl-3-(2-fluoro-4- | | (m, 1H), 2.63-2.53 (m, |
| | (trifluoromethoxy)benzyl)-1-((3R,5S)- | | 1H), 2.50-2.39 (m, 1H), |
| | 5-hydroxy-1-propionylpiperidin-3- | | 2.38-2.17 (m, 2H), 2.17- |
| | yl)urea | | 2.00 (m, 1H), 1.99-1.84 |
| | | | (m, 1H), 0.94-0.85 (m, |
| | | | 2H), 0.77-0.55 (m, 2H). |
| Example 32 | 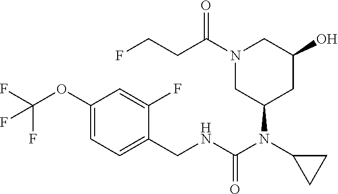 [SMILES: O=C(CCF)N1C[C@@H](O)C[C@@H](N(C(=O)NCc2ccc(OC(F)(F)F)cc2F)C2CC2)C1] | 466.2 | ¹H NMR (400 MHz, dmso) δ 7.48-7.38 (m, 1H), 7.32 (dd, J = 10.5, 2.8 Hz, 1H), 7.22 (d, J = 8.6 Hz, 1H), 6.95 (dt, J = 15.8, 5.9 Hz, 1H), 4.76-4.64 (m, 1H), 4.62-4.52 (m, 1H), 4.44 (dd, J = 12.2, 4.8 Hz, 1H), 4.40-4.21 (m, 2H), 3.67 (dd, J = 12.5, 4.1 Hz, 1H), 3.58 (tt, J = 11.8, 3.9 Hz, 1H), 3.53-3.38 (m, 1H), |
| | 1-cyclopropyl-3-(2-fluoro-4- | | 3.39-3.27 (m, 1H), 3.09 |
| | (trifluoromethoxy)benzyl)-1-((3R,5S)- | | (t, J = 12.0 Hz, 1H), 2.85- |
| | 1-(3-fluoropropanoyl)-5- | | 2.66 (m, 2H), 2.69-2.55 |
| | hydroxypiperidin-3-yl)urea | | (m, 1H), 2.17 (t, J = |
| | | | 11.4 Hz, 1H), 2.09-1.86 (m, |
| | | | 2H), 0.94-0.82 (m, 2H), |
| | | | 0.78-0.58 (m, 2H). |
| Example 38 | 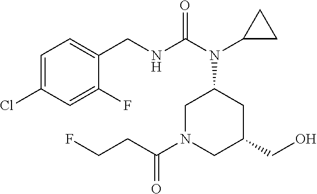 [SMILES: O=C(CCF)N1C[C@@H](CO)C[C@@H](N(C(=O)NCc2ccc(Cl)cc2F)C2CC2)C1] | 430 | |
| | 3-(4-chloro-2-fluorobenzyl)-1- | | |
| | cyclopropyl-1-((3R,5S)-1-(3- | | |
| | fluoropropanoyl)-5- | | |
| | (hydroxymethyl)piperidin-3-yl)urea | | |
| Example 39 | 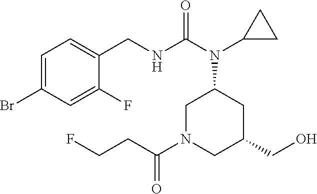 [SMILES: O=C(CCF)N1C[C@@H](CO)C[C@@H](N(C(=O)NCc2ccc(Br)cc2F)C2CC2)C1] | 473.9 | ¹H NMR (400 MHz, dmso) δ 7.48 (dt, J = 9.8, 2.4 Hz, 1H), 7.39 (d, J = 8.2 Hz, 1H), 7.31-7.21 (m, 1H), 6.92 (dt, J = 14.8, 5.9 Hz, 1H), 4.75-4.65 (m, 1H), 4.63-4.54 (m, 1H), 4.53- 4.45 (m, 1H), 4.27 (t, J = 6.2 Hz, 2H), 3.87 (d, J = 13.4 Hz, 1H), 3.72 (d, J = 12.5 Hz, 1H), 3.62-3.42 (m, 1H), 3.38-3.21 (m, |
| | 3-(4-bromo-2-fluorobenzyl)-1- | | 2H), 3.16 (t, J = 12.0 Hz, |
| | cyclopropyl-1-((3R,5S)-1-(3- | | 1H), 2.81-2.66 (m, 2H), |
| | fluoropropanoyl)-5- | | 2.64-2.50 (m, 1H), 2.49- |
| | (hydroxymethyl)piperidin-3-yl)urea | | 2.40 (m, 1H), 2.11 (t, J = |
| | | | 12.2 Hz, 1H), 1.81-1.68 |
| | | | (m, 2H), 0.92-0.81 (m, |
| | | | 2H), 0.74-0.57 (m, 2H). |
| Example 40 | 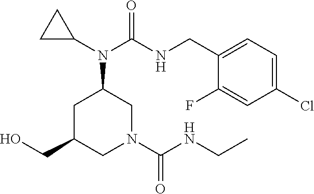 [SMILES: CCNC(=O)N1C[C@@H](CO)C[C@@H](N(C(=O)NCc2ccc(Cl)cc2F)C2CC2)C1] | 427.4 | ¹H NMR (400 MHz, dmso) δ 7.40-7.23 (m, 3H), 6.89 (t, J = 5.9 Hz, 1H), 6.39 (t, J = 5.4 Hz, 1H), 4.51 (t, J = 5.2 Hz, 1H), 4.28 (d, J = 5.8 Hz, 2H), 3.99 (d, J = 11.8 Hz, 1H), 3.81 (d, J = 12.4 Hz, 1H), 3.60-3.46 (m, 1H), 3.25 (q, J = 5.3 Hz, 3H), 3.07-2.96 (m, 2H), 2.91 (t, J = 11.8 Hz, 1H), 2.49-2.39 (m, 1H), |
| | (3R,5S)-3-(3-(4-chloro-2- | | 2.17 (dd, J = 12.9, 11.5 Hz, |
| | fluorobenzyl)-1-cyclopropylureido)-N- | | 1H), 1.76-1.57 (m, 2H), |
| | ethyl-5-(hydroxymethyl)piperidine-1- | | 0.98 (t, J = 7.1 Hz, 3H), |
| | carboxamide | | 0.92-0.83 (m, 2H), 0.66- |
| | | | 0.60 (m, 2H). |
| Example 41 | 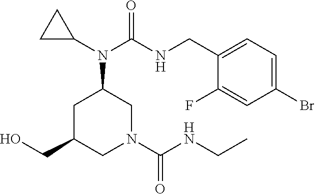 [SMILES: CCNC(=O)N1C[C@@H](CO)C[C@@H](N(C(=O)NCc2ccc(Br)cc2F)C2CC2)C1] | 470.9 | ¹H NMR (400 MHz, dmso) δ 7.48 (dd, J = 9.8, 1.9 Hz, 1H), 7.39 (dd, J = 8.2, 1.9 Hz, 1H), 7.26 (t, J = 8.2 Hz, 1H), 6.89 (t, J = 5.9 Hz, 1H), 6.45-6.32 (m, 1H), 4.52 (s, 1H), 4.26 (d, J = 5.8 Hz, 2H), 4.03-3.95 (m, 1H), 3.85-3.77 (m, 1H), 3.50 (tt, J = 11.5, 4.0 Hz, 1H), 3.32-3.19 (m, 2H), 3.07-2.95 (m, 2H), |
| | (3R,5S)-3-(3-(4-bromo-2- | | 2.90 (t, J = 11.8 Hz, 1H), |
| | fluorobenzyl)-1-cyclopropylureido)-N- | | 2.48-2.39 (m, 1H), 2.24- |
| | ethyl-5-(hydroxymethyl)piperidine-1- | | 2.11 (m, 1H), 1.76-1.57 |
| | carboxamide | | (m, 2H), 1.54-1.51 (m, |
| | | | 1H), 0.98 (t, J = 7.1 Hz, |
| | | | 3H), 0.92-0.79 (m, 2H), |
| | | | 0.67-0.59 (m, 2H). |
| Example 42 | 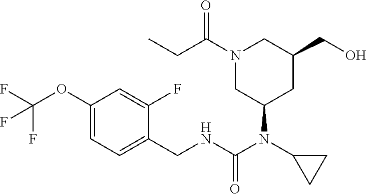 [SMILES: CCC(=O)N1C[C@@H](CO)C[C@@H](N(C(=O)NCc2ccc(OC(F)(F)F)cc2F)C2CC2)C1] | 462.3 | ¹H NMR (400 MHz, dmso) δ 7.47-7.37 (m, 1H), 7.37- 7.29 (m, 1H), 7.23 (d, J = 8.5 Hz, 1H), 7.00-6.88 (m, 1H), 4.31 (t, J = 5.8 Hz, 2H), 3.87 (d, J = 13.4 Hz, 1H), 3.74-3.66 (m, 1H), 3.37-3.19 (m, 3H), 3.11 (t, J = 11.9 Hz, 1H), 2.80 (t, J = 11.7 Hz, 1H), 2.61-2.53 (m, 1H), 2.49- 2.41 (m, 1H), 2.34-2.22 |
| | 1-cyclopropyl-3-(2-fluoro-4- | | (m, 2H), 2.07 (t, J = |
| | (trifluoromethoxy)benzyl)-1-((3R,5S)- | | 12.2 Hz, 1H), 1.84-1.68 (m, |
| | 5-(hydroxymethyl)-1- | | 2H), 0.97 (q, J = 7.1 Hz, |
| | propionylpiperidin-3-yl)urea | | 3H), 0.92-0.81 (m, 2H), |
| | | | 0.72-0.55 (m, 2H). |
| Example 43 | 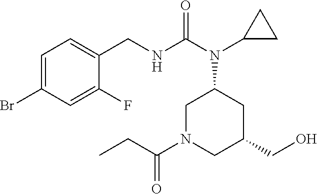 [SMILES: CCC(=O)N1C[C@@H](CO)C[C@@H](N(C(=O)NCc2ccc(Br)cc2F)C2CC2)C1] | 456.2 | ¹H NMR (400 MHz, dmso) δ 7.52-7.43 (m, 1H), 7.42- 7.36 (m, 1H), 7.31-7.21 (m, 1H), 6.91 (dt, J = 15.8, 6.0 Hz, 1H), 4.26 (t, J = 5.7 Hz, 2H), 3.90-3.82 (m, 1H), 3.69 (d, J = 12.7 Hz, 1H), 3.62-3.56 (m, 1H), 3.35-3.21 (m, 2H), 3.10 (t, J = 12.0 Hz, 1H), 2.79 (t, J = 11.7 Hz, 1H), 2.57 (d, J = 12.5 Hz, 1H), 2.46- |
| | 3-(4-bromo-2-fluorobenzyl)-1- | | 2.41 (m, 1H), 2.36-2.22 |
| | cyclopropyl-1-((3R,5S)-5- | | (m, 2H), 2.07 (t, J = |
| | (hydroxymethyl)-1- | | 12.2 Hz, 1H), 1.83-1.66 (m, |
| | propionylpiperidin-3-yl)urea | | 2H), 1.00-0.92 (m, 3H), |
| | | | 0.92-0.79 (m, 2H), 0.73- |
| | | | 0.55 (m, 2H). |
| Example 44 | 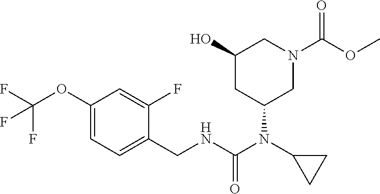 [SMILES: COC(=O)N1C[C@H](O)C[C@@H](N(C(=O)NCc2ccc(OC(F)(F)F)cc2F)C2CC2)C1] | 450.3 | ¹H NMR (400 MHz, dmso) δ 7.42 (t, J = 8.5 Hz, 1H), 7.33 (dd, J = 10.4, 2.4 Hz, 1H), 7.26-7.19 (m, 1H), 6.94 (t, J = 5.9 Hz, 1H), 5.06 (s, 1H), 4.31 (d, J = 5.9 Hz, 2H), 3.99 (s, 1H), 3.80 (s, 1H), 3.57 (s, 3H), 3.45-3.34 (m, 2H), 2.93 (t, J = 11.8 Hz, 1H), 2.49- 2.40 (m, 1H), 2.35 (s, 1H), 2.01-1.92 (m, 1H), 1.87 |
| | methyl (3R,5S)-3-(1-cyclopropyl-3-(2- | | (q, J = 11.6 Hz, 1H), 0.93- |
| | fluoro-4- | | 0.82 (m, 2H), 0.73-0.56 |
| | (trifluoromethoxy)benzyl)ureido)-5- | | (m, 2H). |
| | hydroxypiperidine-1-carboxylate | | |
| Example 45 | 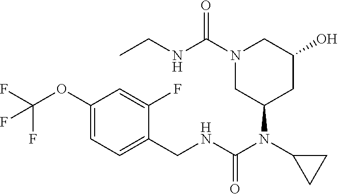 [SMILES: CCNC(=O)N1C[C@H](O)C[C@@H](N(C(=O)NCc2ccc(OC(F)(F)F)cc2F)C2CC2)C1] | 463.3 | ¹H NMR (400 MHz, dmso) δ 7.42 (t, J = 8.5 Hz, 1H), 7.32 (dd, J = 10.4, 2.4 Hz, 1H), 7.23 (dd, J = 8.5, 2.3 Hz, 1H), 6.89 (t, J = 5.9 Hz, 1H), 6.20 (t, J = 5.4 Hz, 1H), 4.56 (s, 1H), 4.31 (d, J = 5.8 Hz, 2H), 3.93-3.79 (m, 4H), 3.08-2.99 (m, 1H), 3.03-2.89 (m, 2H), 2.64 (dd, J = 13.7, 1.7 Hz, 1H), 2.51-2.41 (m, |
| | (3R,5R)-3-(1-cyclopropyl-3-(2-fluoro- | | 1H), 2.15 (td, J = 12.7, |
| | 4-(trifluoromethoxy)benzyl)ureido)-N- | | 2.9 Hz, 1H), 1.71 (d, J = |
| | ethyl-5-hydroxypiperidine-1- | | 12.7 Hz, 1H), 0.99 (t, J = |
| | carboxamide | | 7.1 Hz, 3H), 0.91-0.79 (m, |
| | | | 2H), 0.68-0.59 (m, 2H). |
| Example 46 | 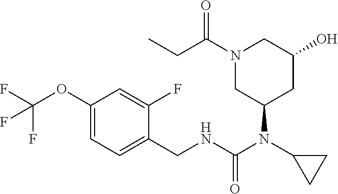 [SMILES: CCC(=O)N1C[C@H](O)C[C@@H](N(C(=O)NCc2ccc(OC(F)(F)F)cc2F)C2CC2)C1] | 448.3 | ¹H NMR (400 MHz, dmso) δ 7.48-7.37 (m, 1H), 7.37- 7.28 (m, 1H), 7.23 (d, J = 8.6 Hz, 1H), 6.97-6.84 (m, 1H), 4.39-4.24 (m, 3H), 4.06-3.88 (m, 2H), 3.80-3.63 (m, 2H), 3.01- 2.86 (m, 2H), 2.55-2.37 (m, 1H), 2.40-2.13 (m, 3H), 1.71 (d, J = 12.9 Hz, 1H), 0.96 (td, J = 7.4, 3.2 Hz, 3H), 0.93-0.81 (m, |
| | 1-cyclopropyl-3-(2-fluoro-4- | | 2H), 0.74-0.55 (m, 2H). |
| | (trifluoromethoxy)benzyl)-1-((3R,5R)- | | |
| | 5-hydroxy-1-propionylpiperidin-3- | | |
| | yl)urea | | |
| Example 47 | 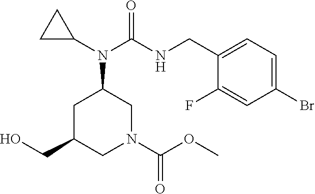 [SMILES: COC(=O)N1C[C@@H](CO)C[C@@H](N(C(=O)NCc2ccc(Br)cc2F)C2CC2)C1] | 458.2 | ¹H NMR (400 MHz, dmso) δ 7.47 (dd, J = 9.8, 2.0 Hz, 1H), 7.39 (dd, J = 8.2, 1.9 Hz, 1H), 7.26 (t, J = 8.2 Hz, 1H), 6.87 (t, J = 5.9 Hz, 1H), 4.57 (s, 1H), 4.26 (d, J = 5.8 Hz, 2H), 4.09-4.02 (m, 1H), 3.88- 3.83 (m, 1H), 3.58 (s, 3H), 3.26-3.17 (m, 1H), 2.99 (t, J = 11.8 Hz, 1H), 2.48-2.38 (m, 1H), 2.33 (s, 1H), 1.73- |
| | methyl (3R,5S)-3-(3-(4-bromo-2- | | 1.58 (m, 2H), 1.61-1.52 |
| | fluorobenzyl)-1-cyclopropylureido)-5- | | (m, 1H), 0.93-0.79 (m, |
| | (hydroxymethyl)piperidine-1- | | 2H), 0.70-0.56 (m, 2H). |
| | carboxylate | | |
| Example 50 | 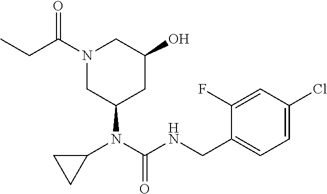 [SMILES: CCC(=O)N1C[C@@H](O)C[C@@H](N(C(=O)NCc2ccc(Cl)cc2F)C2CC2)C1] | 398.3 | ¹H NMR (400 MHz, dmso) δ 7.41-7.23 (m, 3H), 6.97- 6.86 (m, 1H), 4.44 (dd, J = 12.3, 4.7 Hz, 1H), 4.30- 4.21 (m, 2H), 3.67-3.54 (m, 1H), 3.51-3.40 (m, 2H), 3.37-3.26 (m, 1H), 3.03 (t, J = 11.6 Hz, 1H), 2.79-2.67 (m, 1H), 2.63- 2.52 (m, 1H), 2.50-2.38 (m, 1H), 2.38-2.20 (m, 2H), 2.12 (t, J = 11.3 Hz, |
| | 3-(4-chloro-2-fluorobenzyl)-1- | | 1H), 2.07-1.84 (m, 2H), |
| | cyclopropyl-1-((3R,5S)-5-hydroxy-1- | | 0.96 (q, J = 7.0 Hz, 3H), |
| | propionylpiperidin-3-yl)urea | | 0.93-0.84 (m, 2H), 0.76- |
| | | | 0.54 (m, 2H). |
| Example 51 | 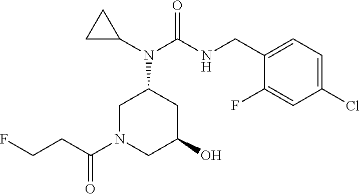 [SMILES: O=C(CCF)N1C[C@H](O)C[C@@H](N(C(=O)NCc2ccc(Cl)cc2F)C2CC2)C1] | 416.6 | ¹H NMR (400 MHz, dmso) δ 7.41-7.23 (m, 3H), 6.98- 6.87 (m, 1H), 5.09 (d, J = 4.6 Hz, 1H), 4.74-4.65 (m, 1H), 4.61-4.53 (m, 1H), 4.44 (dd, J = 12.1, 4.8 Hz, 1H), 4.37-4.21 (m, 2H), 3.81 (dd, J = 13.2, 4.7 Hz, 1H), 3.66 (d, J = 12.7 Hz, 1H), 3.57 (s, 1H), 3.46 (dq, J = 10.6, 5.8 Hz, 1H), 3.09 (t, J = 12.0 Hz, |
| | 3-(4-chloro-2-fluorobenzyl)-1- | | 1H), 2.83-2.68 (m, 2H), |
| | cyclopropyl-1-((3R,5S)-1-(3- | | 2.45 (d, J = 4.5 Hz, 1H), |
| | fluoropropanoyl)-5-hydroxypiperidin- | | 2.16 (t, J = 11.4 Hz, 1H), |
| | 3-yl)urea | | 2.01-1.86 (m, 1H), 0.93- |
| | | | 0.84 (m, 2H), 0.76-0.56 |
| | | | (m, 2H). |
| Example 52 | 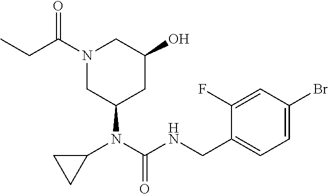 [SMILES: CCC(=O)N1C[C@@H](O)C[C@@H](N(C(=O)NCc2ccc(Br)cc2F)C2CC2)C1] | 442.2 | ¹H NMR (400 MHz, dmso) δ 7.48 (dt, J = 9.9, 2.2 Hz, 1H), 7.39 (dd, J = 8.3, 1.8 Hz, 1H), 7.31-7.21 (m, 1H), 6.97-6.86 (m, 1H), 4.44 (dd, J = 12.0, 4.7 Hz, 1H), 4.25 (dq, J = 12.6, 5.5 Hz, 2H), 3.79 (dd, J = 13.1, 4.7 Hz, 1H), 3.67- 3.53 (m, 1H), 3.51-3.36 (m, 1H), 3.37-3.25 (m, 1H), 3.03 (t, J = 11.7 Hz, 1H), |
| | 3-(4-bromo-2-fluorobenzyl)-1- | | 2.49-2.38 (m, 1H), 2.38- |
| | cyclopropyl-1-((3R,5S)-5-hydroxy-1- | | 2.20 (m, 1H), 2.12 (t, J = |
| | propionylpiperidin-3-yl)urea | | 11.3 Hz, 1H), 2.03 (d, J = |
| | | | 11.8 Hz, 1H), 1.98-1.84 |
| | | | (m, 2H), 1.01-0.93 (m, |
| | | | 3H), 0.96-0.82 (m, 2H), |
| | | | 0.77-0.54 (m, 2H). |
| Example 53 | 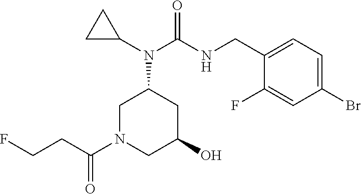 [SMILES: O=C(CCF)N1C[C@H](O)C[C@@H](N(C(=O)NCc2ccc(Br)cc2F)C2CC2)C1] | 460.2 | ¹H NMR (400 MHz, dmso) δ 7.48 (dt, J = 9.8, 2.3 Hz, 1H), 7.43-7.35 (m, 1H), 7.32-7.21 (m, 1H), 6.98- 6.87 (m, 1H), 4.75-4.64 (m, 1H), 4.62-4.53 (m, 1H), 4.44 (dd, J = 12.1, 4.7 Hz, 1H), 4.31-4.20 (m, 2H), 3.81 (dd, J = 13.1, 4.8 Hz, 1H), 3.69-3.53 (m, 1H), 3.53-3.38 (m, 1H), 3.38-3.26 (m, 1H), 3.14- |
| | 3-(4-bromo-2-fluorobenzyl)-1- | | 2.99 (m, 1H), 2.85-2.55 |
| | cyclopropyl-1-((3R,5S)-1-(3- | | (m, 3H), 2.50-2.39 (m, |
| | fluoropropanoyl)-5-hydroxypiperidin- | | 1H), 2.16 (t, J = 11.4 Hz, |
| | 3-yl)urea | | 1H), 1.96-1.86 (m, 1H), |
| | | | 0.93-0.82 (m, 2H), 0.76- |
| | | | 0.55 (m, 2H). |
| Example 54 | 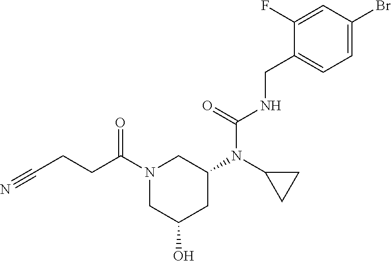 [SMILES: N#CCCC(=O)N1C[C@@H](O)C[C@@H](N(C(=O)NCc2ccc(Br)cc2F)C2CC2)C1] | 467.2 | ¹H NMR (400 MHz, dmso) δ 7.52-7.44 (m, 1H), 7.47- 7.35 (m, 1H), 7.26 (q, J = 7.9 Hz, 1H), 6.97-6.86 (m, 1H), 4.45-4.38 (m, 1H), 4.31-4.24 (m, 2H), 3.83 (d, J = 12.4 Hz, 1H), 3.71-3.64 (m, 1H), 3.60 (d, J = 11.2 Hz, 1H), 3.51- 3.40 (m, 1H), 3.35-3.25 (m, 1H), 3.13-3.02 (m, 1H), 2.80-2.56 (m, 2H), 2.47-2.39 (m, 1H), 2.32- 2.24 (m, 1H), 2.15-2.07 (m, 1H), 2.02 (s, 1H), 1.99- 1.87 (m, 1H), 0.93-0.84 |
| | 3-(4-bromo-2-fluorobenzyl)-1- | | (m, 2H), 0.74-0.59 (m, |
| | ((3R,5S)-1-(3-cyanopropanoyl)-5- | | 2H). |
| | hydroxypiperidin-3-yl)-1- | | |
| | cyclopropylurea | | |
| Example 55 | 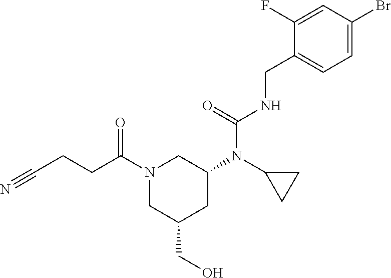 [SMILES: N#CCCC(=O)N1C[C@@H](CO)C[C@@H](N(C(=O)NCc2ccc(Br)cc2F)C2CC2)C1] | 481.1 | |
| | 3-(4-bromo-2-fluorobenzyl)-1- | | |
| | ((3R,5S)-1-(3-cyanopropanoyl)-5- | | |
| | (hydroxymethyl)piperidin-3-yl)-1- | | |
| | cyclopropylurea | | |
| Example 56 | 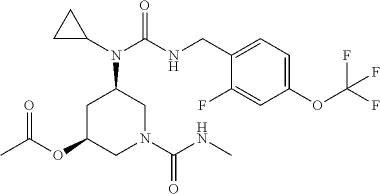 [SMILES: CNC(=O)N1C[C@@H](OC(C)=O)C[C@@H](N(C(=O)NCc2ccc(OC(F)(F)F)cc2F)C2CC2)C1] | 491.3 | ¹H NMR (400 MHz, dmso) δ 7.42 (t, J = 8.5 Hz, 1H), 7.33 (dd, J = 10.3, 2.4 Hz, 1H), 7.26-7.19 (m, 1H), 6.98 (t, J = 5.9 Hz, 1H), 6.51 (q, J = 4.3 Hz, 1H), 4.57-4.45 (m, 1H), 4.31 (d, J = 5.7 Hz, 2H), 4.10 (dd, J = 12.6, 4.9 Hz, 1H), 3.78 (dd, J = 12.6, 4.5 Hz, 1H), 3.62-3.49 (m, 1H), 3.00 (t, J = 11.9 Hz, 1H), |
| | (3S,5R)-5-(1-cyclopropyl-3-(2-fluoro- | | 2.54 (d, J = 4.1 Hz, 3H), |
| | 4-(trifluoromethoxy)benzyl)ureido)-1- | | 2.48-2.41 (m, 2H), 2.11- |
| | (methylcarbamoyl)piperidin-3-yl | | 2.01 (m, 2H), 2.00 (s, 3H), |
| | acetate | | 0.94-0.84 (m, 2H), 0.71- |
| | | | 0.62 (m, 2H). |
| Example 48 | 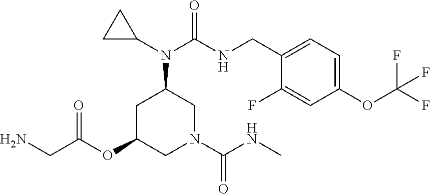 [SMILES: CNC(=O)N1C[C@@H](OC(=O)CN)C[C@@H](N(C(=O)NCc2ccc(OC(F)(F)F)cc2F)C2CC2)C1] | 506.2 | |
| | (3S,5R)-5-(1-cyclopropyl-3-(2-fluoro- | | |
| | 4-(trifluoromethoxy)benzyl)ureido)-1- | | |
| | (methylcarbamoyl)piperidin-3-yl | | |
| | glycinate | | |
| Example 49 | 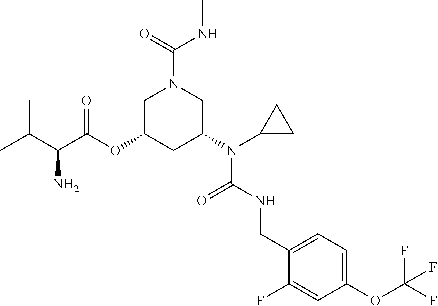 [SMILES: CNC(=O)N1C[C@@H](OC(=O)[C@@H](N)C(C)C)C[C@@H](N(C(=O)NCc2ccc(OC(F)(F)F)cc2F)C2CC2)C1] | 548.3 | |
| | (3S,5R)-5-(1-cyclopropyl-3-(2-fluoro- | | |
| | 4-(trifluoromethoxy)benzyl)ureido)-1- | | |
| | (methylcarbamoyl)piperidin-3-yl L- | | |
| | valinate | | |
| Example 57 | 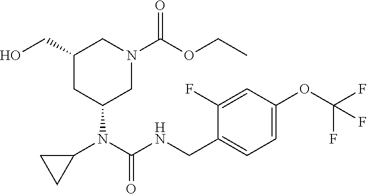 [SMILES: CCOC(=O)N1C[C@@H](CO)C[C@@H](N(C(=O)NCc2ccc(OC(F)(F)F)cc2F)C2CC2)C1] | 478.3 | ¹H NMR (400 MHz, dmso) δ 7.42 (t, J = 8.5 Hz, 1H), 7.33 (dd, J = 10.5, 2.4 Hz, 1H), 7.22 (dt, J = 8.7, 1.5 Hz, 1H), 6.93 (t, J = 5.9 Hz, 1H), 4.59 (t, J = 5.2 Hz, 1H), 4.31 (d, J = 5.8 Hz, 2H), 4.02 (q, J = 7.0 Hz, 2H), 3.87 (s, 1H), 3.65-3.52 (m, 1H), 3.32 (s, 4H), 3.27-3.16 (m, 1H), 2.99 (s, 1H), 2.49-2.39 |
| | ethyl (3R,5S)-3-(1-cyclopropyl-3-(2- | | (m, 1H), 1.73-1.61 (m, |
| | fluoro-4- | | 1H), 1.58-1.53 (m, 1H), |
| | (trifluoromethoxy)benzyl)ureido)-5- | | 1.16 (t, J = 7.1 Hz, 3H), |
| | (hydroxymethyl)piperidine-1- | | 0.94-0.79 (m, 2H), 0.71- |
| | carboxylate | | 0.57 (m, 2H). |
| Example 58 | 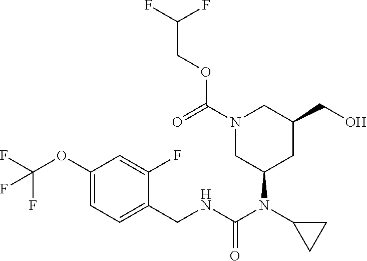 [SMILES: O=C(OCC(F)F)N1C[C@@H](CO)C[C@@H](N(C(=O)NCc2ccc(OC(F)(F)F)cc2F)C2CC2)C1] | 514.1 | ¹H NMR (400 MHz, dmso) δ 7.42 (t, J = 8.5 Hz, 1H), 7.33 (dd, J = 10.3, 2.4 Hz, 1H), 7.22 (dd, J = 8.5, 2.3 Hz, 1H), 6.94 (t, J = 5.9 Hz, 1H), 6.22 (t, J = 3.4 Hz, 1H), 4.62 (s, 1H), 4.34-4.21 (m, 4H), 4.06 (s, 1H), 3.87 (s, 1H), 3.59 (s, 1H), 3.37-3.28 (m, 3H), 3.26-3.20 (m, 1H), 2.49- 2.40 (m, 1H), 1.77-1.64 (m, 2H), 1.64-1.57 (m, 1H), 0.92-0.81 (m, 2H), 0.72-0.57 (m, 2H). |
| | 2,2-difluoroethyl (3R,5S)-3-(1- | | |
| | cyclopropyl-3-(2-fluoro-4- | | |
| | (trifluoromethoxy)benzyl)ureido)-5- | | |
| | (hydroxymethyl)piperidine-1- | | |
| | carboxylate | | |
| Example 59 | 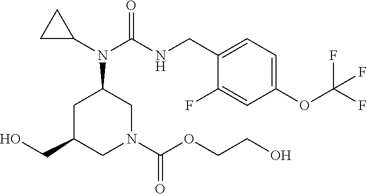 [SMILES: O=C(OCCO)N1C[C@@H](CO)C[C@@H](N(C(=O)NCc2ccc(OC(F)(F)F)cc2F)C2CC2)C1] | 494.1 | ¹H NMR (400 MHz, dmso) δ 7.42 (t, J = 8.6 Hz, 1H), 7.32 (dd, J = 10.4, 2.4 Hz, 1H), 7.22 (dd, J = 8.5, 2.3 Hz, 1H), 6.94 (t, J = 5.9 Hz, 1H), 4.74 (s, 1H), 4.60 (s, 1H), 4.31 (d, J = 5.8 Hz, 2H), 4.08 (dd, J = 13.0, 4.0 Hz, 1H), 3.98 (s, 1H), 3.91 (dd, J = 12.3, 4.2 Hz, 1H), 3.65-3.54 (m, 1H), 3.53 (t, J = 5.2 Hz, |
| | 2-hydroxyethyl (3R,5S)-3-(1- | | 2H), 3.35-3.27 (m, 3H), |
| | cyclopropyl-3-(2-fluoro-4- | | 3.27-3.18 (m, 1H), 2.49- |
| | (trifluoromethoxy)benzyl)ureido)-5- | | 2.40 (m, 1H), 2.33 (s, 1H), |
| | (hydroxymethyl)piperidine-1- | | 1.77-1.59 (m, 2H), 1.62- |
| | carboxylate | | 1.53 (m, 1H), 0.91-0.79 (m, |
| | | | 2H), 0.72-0.58 (m, 2H). |
| Example 64 | 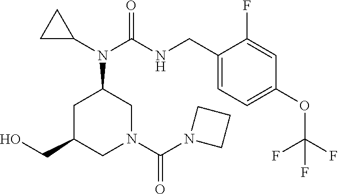 [SMILES: O=C(N1CCC1)N1C[C@@H](CO)C[C@@H](N(C(=O)NCc2ccc(OC(F)(F)F)cc2F)C2CC2)C1] | 489.3 | |
| | 1-((3R,5S)-1-(azetidine-1-carbonyl)-5- | | |
| | (hydroxymethyl)piperidin-3-yl)-1- | | |
| | cyclopropyl-3-(2-fluoro-4- | | |
| | (trifluoromethoxy)benzyl)urea | | |
| Example 62 | 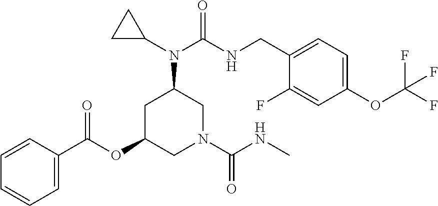 [SMILES: CNC(=O)N1C[C@@H](OC(=O)c2ccccc2)C[C@@H](N(C(=O)NCc2ccc(OC(F)(F)F)cc2F)C2CC2)C1] | 553.3 | ¹H NMR (400 MHz, dmso) δ 8.00-7.93 (m, 2H), 7.72- 7.63 (m, 1H), 7.58-7.49 (m, 2H), 7.47-7.38 (m, 1H), 7.37-7.31 (m, 1H), 7.22 (d, J = 8.3 Hz, 1H), 6.99 (t, J = 5.9 Hz, 1H), 6.60-6.52 (m, 1H), 4.82- 4.72 (m, 1H), 4.32 (d, J = 5.7 Hz, 2H), 4.24 (dd, J = 12.5, 4.9 Hz, 1H), 3.85 (dd, J = 12.4, 4.2 Hz, 1H), 3.69- 3.56 (m, 1H), 3.09 (t, J = |
| | (3S,5R)-5-(1-cyclopropyl-3-(2-fluoro- | | 11.9 Hz, 1H), 2.64 (dd, J = |
| | 4-(trifluoromethoxy)benzyl)ureido)-1- | | 12.6, 10.8 Hz, 1H), 2.57 (d, |
| | (methylcarbamoyl)piperidin-3-yl | | J = 4.1 Hz, 3H), 2.26- |
| | benzoate | | 2.17 (m, 2H), 0.96-0.86 |
| | | | (m, 2H), 0.75-0.65 (m, |
| | | | 2H). |
| Example 63 | 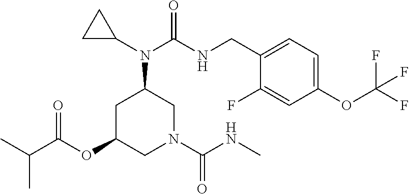 [SMILES: CNC(=O)N1C[C@@H](OC(=O)C(C)C)C[C@@H](N(C(=O)NCc2ccc(OC(F)(F)F)cc2F)C2CC2)C1] | 519.3 | ¹H NMR (400 MHz, dmso) δ 7.42 (t, J = 8.5 Hz, 1H), 7.33 (dd, J = 10.2, 2.4 Hz, 1H), 7.25-7.18 (m, 1H), 6.98 (t, J = 5.9 Hz, 1H), 6.52 (q, J = 4.2 Hz, 1H), 4.57-4.45 (m, 1H), 4.31 (d, J = 5.7 Hz, 2H), 4.08 (dd, J = 12.5, 4.9 Hz, 1H), 3.79 (dd, J = 12.3, 4.2 Hz, 1H), 3.58-3.50 (m, 1H), 3.03 (t, J = 11.9 Hz, 1H), |
| | (3S,5R)-5-(1-cyclopropyl-3-(2-fluoro- | | 2.55 (d, J = 4.2 Hz, 3H), |
| | 4-(trifluoromethoxy)benzyl)ureido)-1- | | 2.48-2.41 (m, 2H), 2.08- |
| | (methylcarbamoyl)piperidin-3-yl | | 1.96 (m, 2H), 1.09 (d, J = |
| | isobutyrate | | 2.7 Hz, 3H), 1.07 (d, J = |
| | | | 2.7 Hz, 3H), 0.94-0.84 |
| | | | (m, 2H), 0.71-0.62 (m, |
| | | | 2H). |
| Example 65 | 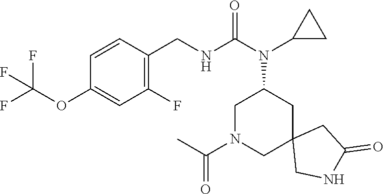 [SMILES: CC(=O)N1C[C@H](N(C(=O)NCc2ccc(OC(F)(F)F)cc2F)C2CC2)CC2(CNC(=O)C2)C1] | 487.2 | 1H NMR (400 MHz, MeOD) δ 7.49-7.37 (m, 1H), 7.09 (t, J = 7.0 Hz, 2H), 4.53 (t, J = 11.8 Hz, 1H), 4.43 (t, J = 10.7 Hz, 2H), 3.90-3.77 (m, 2H), 3.71-3.47 (m, 1H), 3.43- 3.34 (m, 1H), 3.28-3.21 (m, 1H), 3.19 (s, 2H), 3.17- 2.93 (m, 1H), 2.57-2.45 (m, 2H), 2.39-2.22 (m, 2H), 2.20 (d, J = 17.4 Hz, |
| | 1-((9R)-7-acetyl-3-oxo-2,7- | | 1H), 2.13 (d, J = 3.2 Hz, |
| | diazaspiro[4.5]decan-9-yl)-1- | | 2H), 2.09 (d, J = 2.3 Hz, |
| | cyclopropyl-3-(2-fluoro-4- | | 1H), 2.04-1.84 (m, 1H), |
| | (trifluoromethoxy)benzyl)urea | | 1.02-0.92 (m, 2H), 0.85- |
| | | | 0.69 (m, 2H). |
| Example 66 | 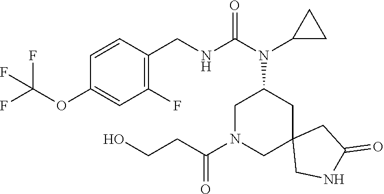 [SMILES: O=C1CC2(CN1)C[C@@H](N(C(=O)NCc1ccc(OC(F)(F)F)cc1F)C1CC1)CN(C(=O)CCO)C2] | 517.2 | 1HNMR (400 MHz, MeOD) δ 7.48-7.39 (m, 1H), 7.09 (t, J = 7.6 Hz, 2H), 7.03-6.92 (m, 1H), 4.57 (t, J = 11.6 Hz, 1H), 4.50-4.37 (m, 0H), 0.99 (d, J = 8.2 Hz, 1H), 3.93-3.72 (m, 3H), 3.61 (d, J = 12.6 Hz, 1H), 3.49-3.32 (m, 1H), 3.25-3.12 (m, 2H), 3.08- 2.94 (m, 1H), 2.74 (dt, J = 14.9, 6.1 Hz, 1H), 2.66- 2.43 (m, 3H), 2.37-2.10 |
| | 1-cyclopropyl-3-(2-fluoro-4- | | (m, 3H), 2.03-1.84 (m, |
| | (trifluoromethoxy)benzyl)-1-((9R)-7- | | 1H), 0.96 (d, J = 6.2 Hz, |
| | (3-hydroxypropanoyl)-3-oxo-2,7- | | 2H), 0.78 (d, J = 12.1 Hz, |
| | diazaspiro[4.5]decan-9-yl)urea | | 2H). |
| Example 67 | 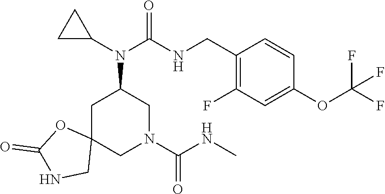 [SMILES: CNC(=O)N1C[C@H](N(C(=O)NCc2ccc(OC(F)(F)F)cc2F)C2CC2)CC2(CNC(=O)O2)C1] | 504.2 | ¹H NMR (400 MHz, MeOD) δ 7.44 (t, J = 8.6 Hz, 1H), 7.14-7.07 (m, 2H), 7.05 (d, J = 5.5 Hz, 1H), 4.45 (t, J = 4.4 Hz, 2H), 4.25 (d, J = 12.9 Hz, 1H), 3.85 (d, J = 12.6 Hz, 1H), 3.78-3.66 (m, 1H), 3.50-3.38 (m, 0H), 0.14 (t, J = 12.0 Hz, 1H), 0.78 (d, J = 12.8 Hz,1H), 0.70 (s, 0H), 2.52 (dd, J = 15.8, 8.9 Hz, |
| | (9R)-9-(1-cyclopropyl-3-(2-fluoro-4- | | 2H), 0.19 (d, J = 10.8 Hz, |
| | (trifluoromethoxy)benzyl)ureido)-N- | | 1H),0.98 (d, J = 6.6 Hz, 0H), |
| | methyl-2-oxo-1-oxa-3,7- | | 0.79 (d, J = 9.0 Hz, 0H). |
| | diazaspiro[4.5]decane-7-carboxamide | | |
| Example 68, | 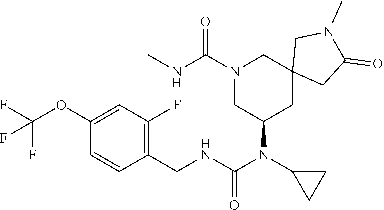 [SMILES: CNC(=O)N1C[C@H](N(C(=O)NCc2ccc(OC(F)(F)F)cc2F)C2CC2)CC2(CC(=O)N(C)C2)C1] | 516.3 | ¹H NMR (400 MHz, MeOD) δ 7.47-7.39 (m, 1H), 7.12-7.04 (m, 2H), 7.03-6.95 (m, 1H), 4.48- 4.39 (m, 2H), 4.02-3.86 (m, 2H), 3.76-3.69 (m, 1H), 3.30-3.25 (m, 1H), 3.23-3.17 (m, 1H), 3.17- 3.07 (m, 1H), 2.83 (s, 3H), 2.72-2.68 (m, 3H), 2.64- 2.55 (m, 1H), 2.55-2.49 (m, 1H), 2.42-2.29 (m, 1H), 2.29-2.17 (m, 2H), |
| | (9R)-9-(1-cyclopropyl-3-(2-fluoro-4- | | 1.96-1.81 (m, 1H), 0.99- |
| | (trifluoromethoxy)benzyl)ureido)-N,2- | | 0.92 (m, 2H), 0.79-0.71 |
| | dimethyl-3-oxo-2,7- | | (m, 2H). |
| | diazaspiro[4.5]decane-7-carboxamide | | |
| Example 69 (rac) | 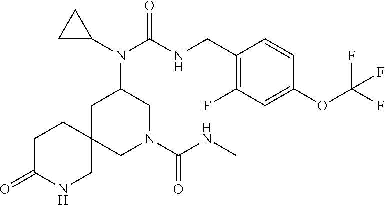 [SMILES: CNC(=O)N1CC(N(C(=O)NCc2ccc(OC(F)(F)F)cc2F)C2CC2)CC2(CCC(=O)NC2)C1] | 516.3 | ¹H NMR (400 MHz, MeOD) δ 7.47-7.38 (m, 1H), 7.14-7.05 (m, 2H), 7.00 (t, J = 5.7 Hz, 1H), 4.51-4.36 (m, 2H), 4.15 (d, J = 13.4 Hz, 1H), 3.97- 3.81 (m, 2H), 3.21 (t, J = 13.1 Hz, 1H), 3.12-2.95 (m, 2H), 2.69 (s, 3H), 2.59- 2.45 (m, 3H), 2.38-2.23 (m, 1H), 2.03 (t, J = 12.5 Hz, 1H), 1.93-1.80 (m, 1H), 1.79-1.62 (m, 2H), |
| | 4-(1-cyclopropyl-3-(2-fluoro-4- | | 1.02-0.90 (m, 2H), 0.81- |
| | (trifluoromethoxy)benzyl)ureido)-N- | | 0.69 (m, 2H). |
| | methyl-9-oxo-2,8- | | |
| | diazaspiro[5.5]undecane-2- | | |
| | carboxamide | | |
| Example 70 | 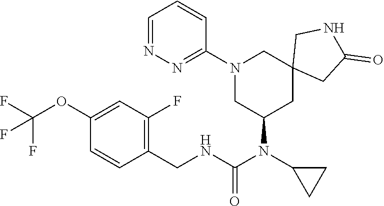 [SMILES: O=C1CC2(CN1)C[C@@H](N(C(=O)NCc1ccc(OC(F)(F)F)cc1F)C1CC1)CN(c1cccnn1)C2] | 523.3 | ¹H NMR (400 MHz, MeOD) δ 8.46 (s, 1H), 7.46 (t, J = 8.6 Hz, 1H), 7.43-7.34 (m, 1H), 7.33- 7.28 (m, 1H), 7.14-7.07 (m, 2H), 7.07-6.98 (m, 1H), 4.59-4.52 (m, 1H), 4.51-4.39 (m, 2H), 4.37- 4.26 (m, 1H), 3.99-3.83 (m, 1H), 3.29-3.17 (m, 3H), 2.87-2.75 (m, 1H), 2.64-2.50 (m, 1H), 2.44- 2.20 (m, 3H), 2.09-1.94 |
| | 1-cyclopropyl-3-(2-fluoro-4- | | (m, 1H), 1.05-0.92 (m, |
| | (trifluoromethoxy)benzyl)-1-((9R)-3- | | 2H), 0.89-0.76 (m, 2H). |
| | oxo-7-(pyridazin-3-yl)-2,7- | | |
| | diazaspiro[4.5]decan-9-yl)urea | | |
| Example 71 | 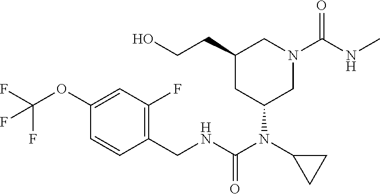 [SMILES: CNC(=O)N1C[C@H](CCO)C[C@@H](N(C(=O)NCc2ccc(OC(F)(F)F)cc2F)C2CC2)C1] | 477.2 | ¹H NMR (400 MHz, MeOD) δ 7.43 (t, J = 8.5 Hz, 1H), 7.09 (t, J = 8.2 Hz, 2H), 4.43 (d, J = 6.7 Hz, 2H), 3.89 (d, J = 7.9 Hz, 3H), 3.64 (t, J = 6.3 Hz, 2H), 3.15 (t, J = 12.5 Hz, 1H), 2.86 (d, J = 11.6 Hz, 1H), 2.69 (s, 3H), 2.50 (s, 1H), 2.33-2.22 (m, 1H), 2.05 (s, 1H), 1.76 (d, J = 12.7 Hz, 1H), 1.67 (dd, |
| | (3R,5S)-3-(1-cyclopropyl-3-(2-fluoro- | | J = 13.6, 7.0 Hz, 1H), 1.53 |
| | 4-(trifluoromethoxy)benzyl)ureido)-5- | | (dd, J = 13.7, 6.6 Hz, 1H), |
| | (2-hydroxyethyl)-N-methylpiperidine- | | 0.94 (d, J = 6.0 Hz, 2H), |
| | 1-carboxamide | | 0.75 (s, 2H). |
| Example 72, | 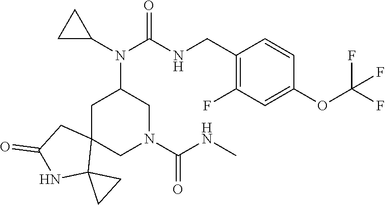 [SMILES: CNC(=O)N1CC(N(C(=O)NCc2ccc(OC(F)(F)F)cc2F)C2CC2)CC2(CC(=O)NC23CC3)C1] | 528.3 | ¹H NMR (400 MHz, MeOD) δ 7.46-7.38 (m, 1H), 7.09 (t, J = 7.3 Hz, 2H), 6.98 (t, J = 5.8 Hz, 1H), 4.48-4.34 (m, 2H), 4.04-3.96 (m, 1H), 3.95- 3.85 (m, 1H), 3.70-3.58 (m, 1H), 3.18-3.08 (m, 1H), 2.70 (s, 3H), 2.59- 2.53 (m, 1H), 2.52-2.31 (m, 3H), 1.86-1.74 (m, 1H), 1.70-1.59 (m, 1H), 0.99-0.91 (m, 2H), 0.86- |
| | 8-(1-cyclopropyl-3-(2-fluoro-4- | | 0.78 (m, 3H), 0.78-0.70 |
| | (trifluoromethoxy)benzyl)ureido)-N- | | (m, 2H), 0.64-0.53 (m, |
| | methyl-11-oxo-6,12- | | 1H). |
| | diazadispiro[2.0.5⁴.3³]dodecane-6- | | |
| | carboxamide | | |
| Example 73 | 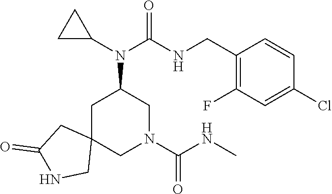 [SMILES: CNC(=O)N1C[C@H](N(C(=O)NCc2ccc(Cl)cc2F)C2CC2)CC2(CNC(=O)C2)C1] | 452.3 | ¹H NMR (400 MHz, MeOD) δ 7.37-7.28 (m, 1H), 7.20-7.12 (m, 2H), 6.99-6.92 (m, 1H), 4.46- 4.34 (m, 2H), 4.14-3.97 (m, 1H), 3.95-3.84 (m, 1H), 3.79-3.63 (m, 1H), 3.29-3.25 (m, 1H), 3.22- 3.18 (m, 1H), 3.17-3.04 (m, 2H), 2.69 (s, 3H), 2.65- 2.55 (m, 1H), 2.55-2.47 (m, 1H), 2.35-2.29 (m, |
| | (9R)-9-(3-(4-chloro-2-fluorobenzyl)-1- | | 1H), 2.28-2.19 (m, 2H), |
| | cyclopropylureido)-N-methyl-3-oxo- | | 2.18-2.03 (m, 1H), 1.98- |
| | 2,7-diazaspiro[4.5]decane-7- | | 1.82 (m, 1H), 1.00-0.90 |
| | carboxamide | | (m, 2H), 0.82-0.63 (m, |
| | | | 2H). |
| Example 74 | 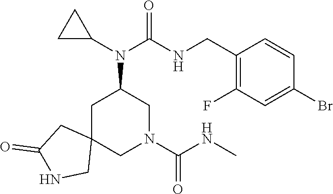 [SMILES: CNC(=O)N1C[C@H](N(C(=O)NCc2ccc(Br)cc2F)C2CC2)CC2(CNC(=O)C2)C1] | 496.2 | 1H NMR (400 MHz, MeOD) δ 7.36-7.21 (m, 3H), 7.00-6.92 (m, 1H), 4.45-4.31 (m, 2H), 4.09- 3.99 (m, 1H), 3.94-3.84 (m, 1H), 3.78-3.65 (m, 1H), 3.28 (s, 0.3H), 3.22- 3.17 (m, 0.7H), 3.17-3.06 (m, 2H), 2.69 (s, 3H), 2.63- 2.58 (m, 1H), 2.55-2.46 (m, 1H), 2.32 (d, J = 16.9 Hz, 0.65H), 2.28- |
| | (9R)-9-(3-(4-bromo-2-fluorobenzyl)-1- | | 2.17 (m, 2H), 2.14-2.08 (m, |
| | cyclopropylureido)-N-methyl-3-oxo- | | 0.38H), 1.97-1.84 (m, |
| | 2,7-diazaspiro[4.5]decane-7- | | 1H), 0.99-0.88 (m, 2H), |
| | carboxamide | | 0.81-0.69 (m, 2H). |
| Example 75 | 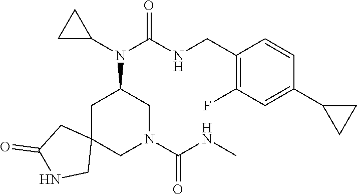 [SMILES: CNC(=O)N1C[C@H](N(C(=O)NCc2ccc(C3CC3)cc2F)C2CC2)CC2(CNC(=O)C2)C1] | 458.3 | ¹H NMR (400 MHz, MeOD) δ 7.18 (t, J = 8.0 Hz, 1H), 6.92-6.58 (m, 3H), 4.41-4.31 (m, 2H), 4.04 (t, J = 13.0 Hz, 1H), 3.91-3.83 (m, 1H), 3.76- 3.65 (m, 1H), 3.21-3.01 (m, 3H), 2.69 (d, J = 0.9 Hz, 3H), 2.62-2.46 (m, 2H), 2.32-2.09 (m, 3H), 1.93-1.81 (m, 2H), 0.98- 0.87 (m, 4H), 0.74-0.60 |
| | (9R)-9-(1-cyclopropyl-3-(4- | | (m, 4H). |
| | cyclopropyl-2-fluorobenzyl)ureido)-N- | | |
| | methyl-3-oxo-2,7- | | |
| | diazaspiro[4.5]decane-7-carboxamide | | |
| Example 76 | 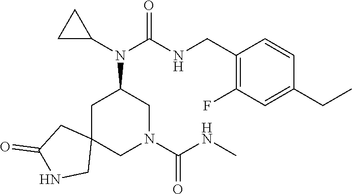 [SMILES: CCc1ccc(CNC(=O)N(C2CC2)[C@H]2CN(C(=O)NC)CC3(CNC(=O)C3)C2)c(F)c1] | 446.3 | ¹H NMR (400 MHz, MeOD) δ 7.22 (t, J = 7.9 Hz, 1H), 6.97 (d, J = 7.8 Hz, 1H), 6.91 (d, J = 11.5 Hz, 1H), 4.38 (dd, J = 15.0, 9.2 Hz, 2H), 4.05 (t, J = 13.2 Hz, 1H), 3.93-3.85 (m, 1H), 3.79-3.68 (m, 1H), 3.22-3.05 (m, 3H), 2.69 (d, J = 1.0 Hz, 3H), 2.62 (dt, J = 11.5, 5.7 Hz, 3H), 2.54-2.46 (m, 1H), 2.30 (s, 1H), 2.28-2.19 |
| | (9R)-9-(1-cyclopropyl-3-(4-ethyl-2- | | (m, 2H), 2.11 (d, J = |
| | fluorobenzyl)ureido)-N-methyl-3-oxo- | | 17.2 Hz, 1H), 1.91 (dd, |
| | 2,7-diazaspiro[4.5]decane-7- | | J = 22.2, 12.6 Hz, 1H), |
| | carboxamide | | 1.21 (t, J = 7.6 Hz, 3H), |
| | | | 0.94 (d, J = 6.4 Hz, 2H), |
| | | | 0.78-0.69 (m, 2H). |
| Example 77 | 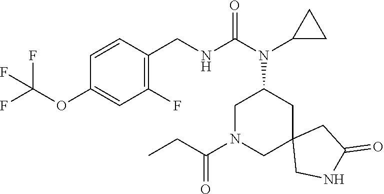 [SMILES: CCC(=O)N1C[C@H](N(C(=O)NCc2ccc(OC(F)(F)F)cc2F)C2CC2)CC2(CNC(=O)C2)C1] | 501.2 | ¹H NMR (400 MHz, MeOD) δ 7.45 (t, J = 8.5 Hz, 1H), 7.09 (t, J = 8.1 Hz, 2H), 4.55 (d, J = 12.0 Hz, 1H), 4.45 (d, J = 6.9 Hz, 2H), 3.99-3.76 (m, 2H), 3.61 (d, J = 15.4 Hz, 1H), 3.37 (d, J = 12.0 Hz, 1H), 3.29-3.21 (m, 1H), 3.21-2.93 (m, 2H), 2.60- 2.44 (m, 3H), 2.42-2.13 (m, 4H), 2.04-1.84 (m, |
| | 1-cyclopropyl-3-(2-fluoro-4- | | 1H), 1.16-1.08 (m, 3H), |
| | (trifluoromethoxy)benzyl)-1-((9R)-3- | | 1.03-0.92 (m, 2H), 0.86- |
| | oxo-7-propionyl-2,7- | | 0.69 (m, 2H). |
| | diazaspiro[4.5]decan-9-yl)urea | | |
| Example 78 | 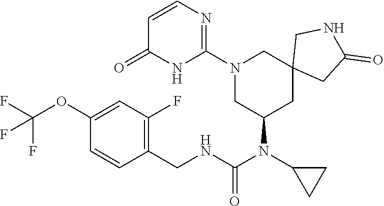 [SMILES: O=C1CC2(CN1)C[C@@H](N(C(=O)NCc1ccc(OC(F)(F)F)cc1F)C1CC1)CN(c1nccc(=O)[nH]1)C2] | 539.2 | ¹H NMR (400 MHz, MeOD) δ 7.63 (s, 1H), 7.45 (t, J = 8.6 Hz, 1H), 7.09 (t, J = 8.5 Hz, 2H), 5.76 (d, J = 6.6 Hz, 1H), 4.53 (s, 1H), 4.43 (t, J = 10.8 Hz, 2H), 4.28 (s, 1H), 3.82 (d, J = 8.5 Hz, 1H), 3.24 (dd, J = 28.7, 6.9 Hz, 3H), 2.81-2.72 (m, 1H), 2.61-2.51 (m, 1H), 2.37- 2.14 (m, 3H), 1.98 (dd, J = 21.9, 12.5 Hz, 1H), 0.98 (d, |
| | 1-cyclopropyl-3-(2-fluoro-4- | | J = 6.2 Hz, 2H), 0.80 (s, |
| | (trifluoromethoxy)benzyl)-1-((9R)-3- | | 2H). |
| | oxo-7-(6-oxo-1,6-dihydropyrimidin-2- | | |
| | yl)-2,7-diazaspiro[4.5]decan-9-yl)urea | | |
| Example 79 | 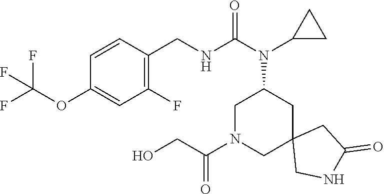 [SMILES: O=C1CC2(CN1)C[C@@H](N(C(=O)NCc1ccc(OC(F)(F)F)cc1F)C1CC1)CN(C(=O)CO)C2] | 503.2 | ¹H NMR (400 MHz, MeOD) δ 7.44 (t, J = 8.4 Hz, 1H), 7.09 (t, J = 7.7 Hz, 2H), 4.48 (dt, J = 27.0,19.5 Hz, 4H), 4.33 (dd, J = 15.1, 4.5 Hz, 1H), 4.20 (d, J = 15.0 Hz, 1H), 3.83-3.59 (m, 2H), 3.25- 3.15 (m, 2H), 2.93 (t, J = 13.5 Hz,1H), 0.63-2.45 (m, 2H), 2.37-2.13 (m, 3H), 2.04-1.85 (m, 1H), 0.96 |
| | 1-cyclopropyl-3-(2-fluoro-4- | | (d, J = 4.4 Hz, 2H), 0.77 |
| | (trifluoromethoxy)benzyl)-1-((9R)-7- | | (d, J = 12.7 Hz, 2H) |
| | (2-hydroxyacetyl)-3-oxo-2,7- | | |
| | diazaspiro[4.5]decan-9-yl)urea | | |
| Example 80 | 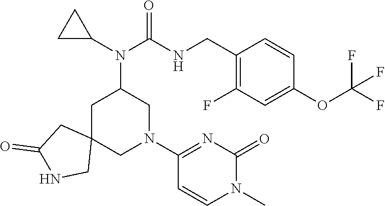 [SMILES: Cn1ccc(N2CC(N(C(=O)NCc3ccc(OC(F)(F)F)cc3F)C3CC3)CC3(CNC(=O)C3)C2)nc1=O] | 553.3 | ¹H NMR (400 MHz, MeOD) δ 7.62 (d, J = 7.5 Hz, 1H), 7.44 (t, J = 8.6 Hz, 1H), 7.09 (t, J = 8.0 Hz, 2H), 7.02 (d, J = 6.0 Hz, 1H), 6.25-6.16 (m, 1H), 4.83-4.74 (m, 1H), 4.44 (d, J = 4.8 Hz, 2H), 4.02 (s, 1H), 3.74 (s, 1H), 3.37 (s, 3H), 3.25 (s, 1H), 3.24-3.12 (m, 2H), 2.78 (s, 1H), 2.58 (s, 1H), 2.35 (t, J = 12.8 Hz, 1H), 2.25 |
| | 1-cyclopropyl-3-(2-fluoro-4- | | (q, J = 17.1 Hz, 2H), 1.98 |
| | (trifluoromethoxy)benzyl)-1-((9R)-7- | | (t, J = 17.2 Hz, 1H), 0.97 |
| | (1-methyl-2-oxo-1,2- | | (d, J = 3.7 Hz, 2H), 0.80 (s, |
| | dihydropyrimidin-4-yl)-3-oxo-2,7- | | 2H). |
| | diazaspiro[4.5]decan-9-yl)urea | | |
| Example 81 (cis) | 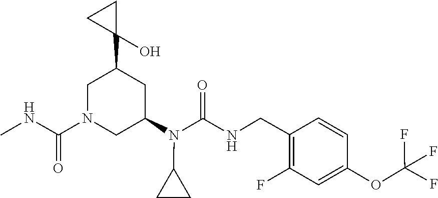 [SMILES: CNC(=O)N1C[C@H](N(C(=O)NCc2ccc(OC(F)(F)F)cc2F)C2CC2)C[C@H](C2(O)CC2)C1] | 489.3 | ¹H NMR (400 MHz, MeOD) δ 7.44 (t, J = 8.3 Hz, 1H), 7.13-7.05 (m, 2H), 6.97 (s, 1H), 4.50- 4.38 (m, 2H), 4.13-4.02 (m, 1H), 3.88-3.79 (m, 1H), 3.75-3.62 (m, 1H), 3.16-3.05 (m, 1H), 2.76- 2.70 (m, 1H), 2.69 (s, 3H), 2.57-2.48 (m, 1H), 2.28- 2.14 (m, 1H), 1.97-1.87 (m, 1H), 1.18-1.06 (m, 1H), 1.01-0.91 (m, 2H), |
| | (3R,5S)-3-(1-cyclopropyl-3-(2-fluoro- | | 0.82-0.73 (m, 2H), 0.72- |
| | 4-(trifluoromethoxy)benzyl)ureido)-5- | | 0.60 (m, 2H), 0.60-0.52 |
| | (1-hydroxycyclopropyl)-N- | | (m, 1H), 0.52-0.43 (m, |
| | methylpiperidine-1-carboxamide | | 1H). |
| Example 82 | 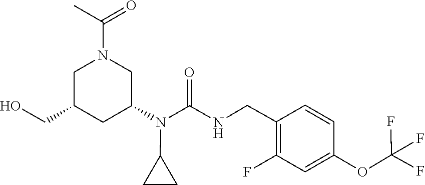 [SMILES: CC(=O)N1C[C@@H](CO)C[C@@H](N(C(=O)NCc2ccc(OC(F)(F)F)cc2F)C2CC2)C1] | 448.2 | ¹H NMR (400 MHz, MeOD) δ 7.48-7.38 (m, 1H), 7.14-7.04 (m, 2H), 7.02-6.90 (m, 1H), 4.69- 4.54 (m, 1H), 4.52-4.35 (m, 2.5H), 3.97 (d, J = 9.9 Hz, 0.5H), 3.89-3.74 (m, 1H), 3.63-3.35 (m, 2.5H), 3.29-3.23 (m, 0.5H), 3.06 (t, J = 11.9 Hz, 0.5H), 2.80-2.68 (m, 0.5H), 2.62-2.47 (m, 1H), 2.31- |
| | 1-((3R,5S)-1-acetyl-5- | | 2.19 (m, 0.5H), 2.11 (s, 3H), |
| | (hydroxymethyl)piperidin-3-yl)-1- | | 2.03-1.82 (m, 2H), 1.80- |
| | cyclopropyl-3-(2-fluoro-4- | | 1.59 (m, 1H), 1.02-0.88 |
| | (trifluoromethoxy)benzyl)urea | | (m, 2H), 0.85-0.66 (m, |
| | | | 2H). |
| Example 83 (cis) | 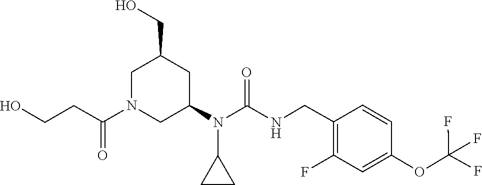 [SMILES: O=C(CCO)N1C[C@@H](CO)C[C@@H](N(C(=O)NCc2ccc(OC(F)(F)F)cc2F)C2CC2)C1] | 478.2 | ¹H NMR (400 MHz, CD3OD) δ 7.48-7.40 (m, 1H), 7.09 (t, J = 9.0 Hz, 2H), 7.01-6.91 (m, 1H), 4.70-4.63 (m, 1H), 4.53- 4.48 (m, 1H), 4.47-4.38 (m, 2H), 4.07 (d, J = 10.2 Hz, 1H), 3.93 (d, J = 12.8 Hz, 1H), 3.87-3.69 (m, 2H), 3.60-3.38 (m, 2H), 3.32 (s, 1H), 3.09 (t, J = 11.9 Hz, 1H), 2.72-2.51 |
| | 1-cyclopropyl-3-(2-fluoro-4- | | (m, 3H), 2.26 (t, J = |
| | (trifluoromethoxy)benzyl)-1-((3R,5S)- | | 12.3 Hz, 1H), 2.00-1.83 |
| | 5-(hydroxymethyl)-1-(3- | | (m, 2H), 1.81-1.63 (m, 1H), |
| | hydroxypropanoyl)piperidin-3-yl)urea | | 1.01-0.90 (m, 2H), 0.86- |
| | | | 0.68 (m, 2H). |
| Example 86 | 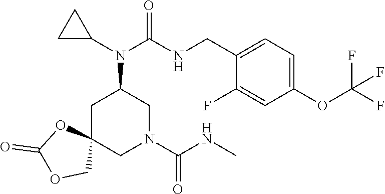 [SMILES: CNC(=O)N1C[C@H](N(C(=O)NCc2ccc(OC(F)(F)F)cc2F)C2CC2)C[C@@]2(COC(=O)O2)C1] | 505.1 | 1HNMR (400 MHz, MeOD) δ 7.43 (t, J = 7.9 Hz,1H), 7.08 (d, J = 9.4 Hz, 2H), 4.50-4.26 (m, 5H), 3.84 (d, J = 12.2 Hz, 1H), 0.65 (s, 1H), 0.17 (t, J = 12.1 Hz,1H), 2.88 (d, J = 12.9 Hz,1H), 0.70 (s, 0H), 0.62 (t, J = 12.5 Hz, 1H), 0.53 (s, 1H), 0.25 (d, J = 12.4 Hz, 1H),0.98 (d, J = 6.0 Hz, 2H),0.78 (d, J = 8.8 Hz, 0H). |
| | (5S,9R)-9-(1-cyclopropyl-3-(2-fluoro- | | |
| | 4-(trifluoromethoxy)benzyl)ureido)-N- | | |
| | methyl-2-oxo-1,3-dioxa-7- | | |
| | azaspiro[4.5]decane-7-carboxamide | | |
| Example 87 | 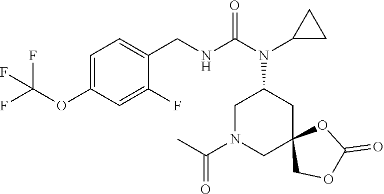 [SMILES: CC(=O)N1C[C@H](N(C(=O)NCc2ccc(OC(F)(F)F)cc2F)C2CC2)C[C@]2(COC(=O)O2)C1] | 490.2 | ¹H NMR (400 MHz, MeOD) δ 7.45 (t, J = 8.5 Hz, 1H), 7.13-7.04 (m, 2H), 4.73 (d, J = 12.7 Hz, 1H), 4.49-4.39 (m, 3H), 4.31 (dd, J = 20.4, 8.6 Hz, 2H), 4.14 (d, J = 13.8 Hz, 1H), 3.86 (d, J = 12.9 Hz, 1H), 3.76 (t, J = 12.3 Hz, 1H), 3.57 (s, 1H), 3.41 (dd, J = 30.3, 18.3 Hz, 1H), 3.09 (d, J = 30.0 Hz, 1H), |
| | 1-((5S,9R)-7-acetyl-2-oxo-1,3-dioxa-7- | | 2.80 (d, J = 12.9 Hz, 1H), |
| | azaspiro[4.5]decan-9-yl)-1- | | 2.68 (t, J = 12.6 Hz, 1H), |
| | cyclopropyl-3-(2-fluoro-4- | | 2.54 (s, 1H), 2.32 (d, J = |
| | (trifluoromethoxy)benzyl)urea | | 11.7 Hz, 1H), 2.25 (d, J = |
| | | | 12.9 Hz, 1H), 2.14 (s, 3H), |
| | | | 1.04-0.90 (m, 2H), 0.87- |
| | | | 0.70 (m, 2H). |
| Example 88 (trans) | 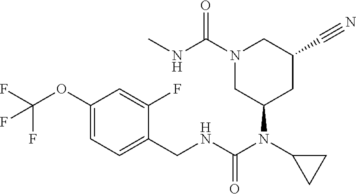 [SMILES: CNC(=O)N1C[C@H](C#N)C[C@@H](N(C(=O)NCc2ccc(OC(F)(F)F)cc2F)C2CC2)C1] | 458.2 | 1HNMR (400 MHz, MeOD) δ 7.43 (t, J = 8.6 Hz,1H), 7.09 (t, J = 7.6 Hz, 2H), 4.49-4.38 (m, 2H), 4.31 (d, J = 13.9 Hz, 1H), 3.94 (d, J = 12.6 Hz,1H), 3.85 (dt, J = 16.1, 3.9 Hz, 1H), 3.22 (dd, J = 17.6, 7.2 Hz, 2H), 2.88 (dd, J = 13.9, 2.7 Hz, 1H), 2.72 (s, 3H), 2.65-2.58 (m, 1H), 2.52 (td, J = 12.9, 4.6 Hz, 1H), |
| | (3R,5R)-3-cyano-5-(1-cyclopropyl-3- | | 2.10 (d, J = 12.9 Hz, 1H), |
| | (2-fluoro-4- | | 0.97 (d, J = 4.7 Hz, 2H), |
| | (trifluoromethoxy)benzyl)ureido)-N- | | 0.79 (d, J = 3.2 Hz, 2H). |
| | methylpiperidine-1-carboxamide | | |
| Example 89 (rac) | 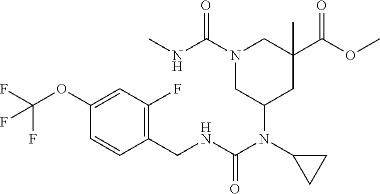 [SMILES: CNC(=O)N1CC(N(C(=O)NCc2ccc(OC(F)(F)F)cc2F)C2CC2)CC(C)(C(=O)OC)C1] | 505.2 | ¹H NMR (400 MHz, MeOD) δ 7.43 (t, J = 8.6 Hz, 1H), 7.09 (m, 2H), 4.50-4.37 (m, 2H), 4.33 (d, J = 13.4 Hz, 0.7H), 4.03 (d, J = 13.2 Hz, 0.3H), 3.97- 3.84 (m, 1.3H), 3.71-3.67 (m, 3H), 3.66-3.59 (m, 07H), 3.19-3.10 (m, 1H), 2.93 (d, J = 13.8 Hz, 0.3H), 2.69 (s, 3H), 2.64-2.50 (m, 1.7H), 2.41 (t, J = 12.4 Hz, |
| | methyl 5-(1-cyclopropyl-3-(2-fluoro-4- | | 0.3H), 2.29-2.20 (m, 0.7H), |
| | (trifluoromethoxy)benzyl)ureido)-3- | | 2.10 (t, J = 12.6 Hz, 0.7H), |
| | methyl-1- | | 1.87 (d, J = 13.1 Hz, 0.3H), |
| | (methylcarbamoyl)piperidine-3- | | 1.27-1.15 (m, 3H), 1.02- |
| | carboxylate | | 0.89 (m, 2H), 0.86-0.68 |
| | | | (m, 2H). |
| Example 90 | 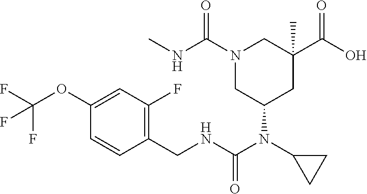 [SMILES: CNC(=O)N1C[C@@H](N(C(=O)NCc2ccc(OC(F)(F)F)cc2F)C2CC2)C[C@](C)(C(=O)O)C1] | 491.2 | ¹H NMR (400 MHz, MeOD) δ 7.43 (t, J = 8.4 Hz, 1H), 7.15-7.00 (m, 2H), 6.93 (s, 1H), 4.51- 4.35 (m, 2H), 4.29 (d, J = 13.2 Hz, 1H), 4.05-3.90 (m, 1H), 3.73-3.57 (m, 1H), 3.13 (t, J = 11.9 Hz, 1H), 2.69 (s, 3H), 2.64- 2.57 (m, 1H), 2.52 (d, J = 13.5 Hz, 1H), 2.25 (d, J = 12.6 Hz, 1H), 2.09 (t, J = |
| | (3S,5S)-5-(1-cyclopropyl-3-(2-fluoro-4- | | 12.4 Hz, 1H), 1.19 (s, 3H), |
| | (trifluoromethoxy)benzyl)ureido)-3- | | 1.01-0.86 (m, 2H), 0.86- |
| | methyl-1- | | 0.77 (m, 1H), 0.77-0.67 |
| | (methylcarbamoyl)piperidine-3- | | (m, 1H). |
| | carboxylic acid | | |
| Example 91 | 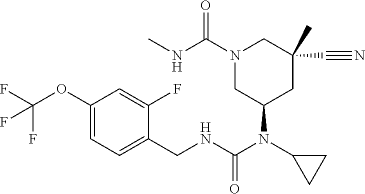 [SMILES: CNC(=O)N1C[C@H](N(C(=O)NCc2ccc(OC(F)(F)F)cc2F)C2CC2)C[C@@](C)(C#N)C1] | 472.3 | ¹H NMR (400 MHz, MeOD) δ 7.43 (t, J = 8.4 Hz, 1H), 7.15-7.01 (m, 2H), 4.51-4.35 (m, 2H), 4.28 (d, J = 13.7 Hz, 1H), 3.97 (d, J = 12.6 Hz, 1H), 3.91-3.77 (m, 1H), 3.23 (t, J = 12.0 Hz, 1H), 2.72 (s, 3H), 2.67-2.56 (m, 2H), 2.28 (t, J = 12.7 Hz, 1H), 2.17-2.06 (m, 1H), 1.37 (s, 3H), 1.04-0.91 |
| | (3R,5R)-3-cyano-5-(1-cyclopropyl-3- | | (m, 2H), 0.85-0.70 (m, |
| | (2-fluoro-4- | | 2H). |
| | (trifluoromethoxy)benzyl)ureido)-N,3- | | |
| | dimethylpiperidine-1-carboxamide | | |
| Example 92 | 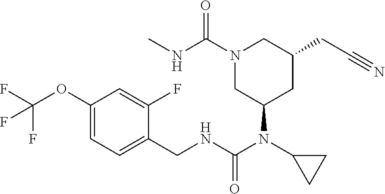 [SMILES: CNC(=O)N1C[C@H](CC#N)C[C@@H](N(C(=O)NCc2ccc(OC(F)(F)F)cc2F)C2CC2)C1] | 472.1 | ¹H NMR (400 MHz, MeOD) δ 7.43 (t, J = 8.6 Hz, 1H), 7.09 (t, J = 7.6 Hz, 2H), 4.49-4.37 (m, 2H), 3.96 (d, J = 13.7 Hz, 1H), 3.81 (t, J = 12.8 Hz, 2H), 3.23 (d, J = 12.2 Hz, 1H), 2.95 (dd, J = 13.7, 3.1 Hz, 1H), 2.70 (s, 3H), 2.54 (ddd, J = 10.0, 7.2, 3.2 Hz, 3H), 2.39 (dd, J = 32.5, 3.7 Hz, 2H), 1.90 (d, |
| | (3S,5R)-3-(cyanomethyl)-5-(1- | | J = 13.0 Hz, 1H), 0.96 (d, |
| | cyclopropyl-3-(2-fluoro-4- | | J = 6.6 Hz, 2H), 0.81-0.69 |
| | (trifluoromethoxy)benzyl)ureido)-N- | | (m, 2H). |
| | methylpiperidine-1-carboxamide | | |
| Example 93 | 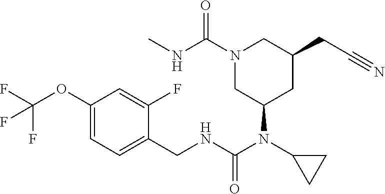 [SMILES: CNC(=O)N1C[C@@H](CC#N)C[C@@H](N(C(=O)NCc2ccc(OC(F)(F)F)cc2F)C2CC2)C1] | 472.3 | ¹H NMR (400 MHz, MeOD) δ 7.43 (t, J = 8.6 Hz, 1H), 7.09 (t, J = 7.6 Hz, 2H), 4.44 (s, 2H), 4.11 (d, J = 12.4 Hz, 1H), 3.85 (d, J = 12.9 Hz, 1H), 3.68 (dd, J = 8.4, 4.0 Hz, 1H), 3.16 (t, J = 12.1 Hz, 1H), 2.70 (s, 3H), 2.58-2.53 (m, 1H), 0.53-2.40 (m, 3H), 2.07-1.86 (m, 3H), 0.96 (d, J = 3.6 Hz, 2H), 0.77 (d, J = |
| | (3R,5R)-3-(cyanomethyl)-5-(1- | | 2.4 Hz, 2H). |
| | cyclopropyl-3-(2-fluoro-4- | | |
| | (trifluoromethoxy)benzyl)ureido)-N- | | |
| | methylpiperidine-1-carboxamide | | |
| Example 94 (cis) | 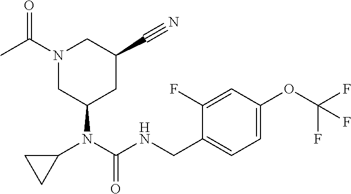 [SMILES: CC(=O)N1C[C@@H](C#N)C[C@@H](N(C(=O)NCc2ccc(OC(F)(F)F)cc2F)C2CC2)C1] | 443.1 | ¹H NMR (400 MHz, MeOD) δ 7.44 (dd, J = 15.5, 8.1 Hz, 1H), 7.09 (t, J = 8.3 Hz, 2H), 7.04-6.95 (m, 1H), 4.74 (d, J = 11.9 Hz, 1H), 4.44 (s, 2H), 4.12 (dd, J = 36.3, 13.9 Hz, 1H), 3.83 (d, J = 13.0 Hz, 1H), 3.71 (d, J = 12.3 Hz, 1H), 3.44 (t, J = 12.2 Hz, 1H), 3.26-3.06 (m, 1H), 2.98 (s, 1H), 2.84 (s, 1H), |
| | 1-((3R,5S)-1-acetyl-5-cyanopiperidin- | | 2.71 (t, J = 12.3 Hz, 1H), |
| | 3-yl)-1-cyclopropyl-3-(2-fluoro-4- | | 2.54 (ddd, J = 40.0, 20.5, |
| | (trifluoromethoxy)benzyl)urea | | 9.6 Hz, 2H), 2.35-2.21 (m, |
| | | | 1H), 2.12 (d, J = 11.7 Hz, |
| | | | 3H), 1.05-0.89 (m, 2H), |
| | | | 0.88-0.62 (m, 2H). |
| Example 95 | 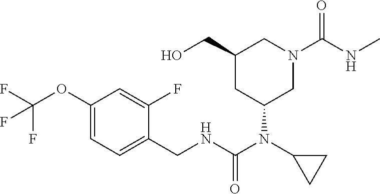 [SMILES: CNC(=O)N1C[C@H](CO)C[C@@H](N(C(=O)NCc2ccc(OC(F)(F)F)cc2F)C2CC2)C1] | 463.2 | ¹H NMR (400 MHz, MeOD) δ 7.43 (t, J = 8.6 Hz, 1H), 7.09 (t, J = 7.9 Hz, 2H), 4.49-4.35 (m, 2H), 3.99-3.72 (m, 3H), 3.53 (dt, J = 17.8, 10.6 Hz, 2H), 3.17 (t, J = 11.8 Hz, 1H), 2.89 (d, J = 13.8 Hz, 1H), 2.70 (s, 3H), 2.51 (s, 1H), 2.26 (td, J = 13.0, 5.3 Hz, 1H), 2.01 (s, 1H), 1.87 (d, J = 12.7 Hz, 1H), |
| | (3R,5R)-3-(1-cyclopropyl-3-(2-fluoro- | | 0.94 (d, J = 6.5 Hz, 2H), |
| | 4-(trifluoromethoxy)benzyl)ureido)-5- | | 0.75 (s, 2H). |
| | (hydroxymethyl)-N-methylpiperidine- | | |
| | 1-carboxamide | | |
| Example 96 | 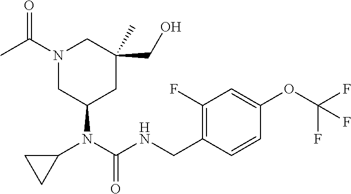 [SMILES: CC(=O)N1C[C@H](N(C(=O)NCc2ccc(OC(F)(F)F)cc2F)C2CC2)C[C@](C)(CO)C1] | 462.3 | ¹H NMR (400 MHz, MeOD) δ 7.48-7.37 (m, 1H), 7.14-7.01 (m, 2H), 7.00-6.87 (m, 1H), 4.50- 4.34 (m, 2.5H), 4.25 (d, J = 14.2 Hz, 0.5H), 3.99-3.87 (m, 0.5H), 3.86-3.73 (m, 1H), 3.73-3.64 (m, 0.5H), 3.50-3.45 (m, 0.5H), 3.35- 3.33 (m, 1H), 3.29-3.24 (m, 1H), 3.04 (t, J = 12.2 Hz, 0.5H), 2.77 (d, |
| | 1-((3R,5S)-1-acetyl-5- | | J = 13.1 Hz, 0.5H), 2.58- |
| | (hydroxymethyl)-5-methylpiperidin-3- | | 2.46 (m, 1H), 2.37 (d, J = |
| | yl)-1-cyclopropyl-3-(2-fluoro-4- | | 13.7 Hz, 0.5H), 2.09 (d, J = |
| | (trifluoromethoxy)benzyl)urea | | 20.8 Hz, 3H), 2.06-1.98 |
| | | | (m, 0.5H), 1.97-1.85 (m, |
| | | | 1H), 1.69 (d, J = 13.7 Hz, |
| | | | 0.5H), 1.02-0.89 (m, 5H), |
| | | | 0.84-0.64 (m, 2H). |
| Example 97 | 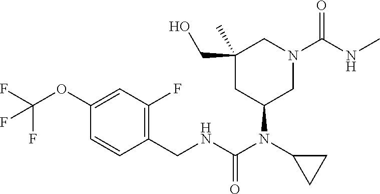 [SMILES: CNC(=O)N1C[C@@H](N(C(=O)NCc2ccc(OC(F)(F)F)cc2F)C2CC2)C[C@@](C)(CO)C1] | 477.3 | ¹H NMR (400 MHz, MeOD) δ 7.43 (t, J = 8.1 Hz, 1H), 7.14-7.02 (m, 2H), 4.49-4.36 (m, 2H), 3.94-3.86 (m, 1H), 3.86- 3.75 (m, 2H), 3.45-3.32 (m, 2H), 3.12 (t, J = 11.9 Hz, 1H), 2.69 (s, 3H), 2.58-2.41 (m, 2H), 1.91 (t, J = 12.9 Hz, 1H), 1.82- 1.71 (m, 1H), 0.99-0.90 (m, 5H), 0.80-0.70 (m, 2H). |
| | (3R,5S)-5-(1-cyclopropyl-3-(2-fluoro- | | |
| | 4-(trifluoromethoxy)benzyl)ureido)-3- | | |
| | (hydroxymethyl)-N,3- | | |
| | dimethylpiperidine-1-carboxamide | | |
| Example 98 | 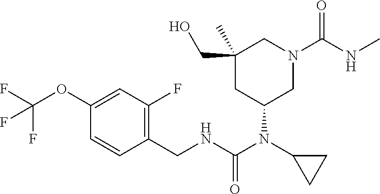 [SMILES: CNC(=O)N1C[C@H](N(C(=O)NCc2ccc(OC(F)(F)F)cc2F)C2CC2)C[C@@](C)(CO)C1] | 477.5 | ¹H NMR (400 MHz, MeOD) δ 7.43 (t, J = 8.3 Hz, 1H), 7.15-7.02 (m, 2H), 7.00-6.91 (m, 1H), 4.51-4.36 (m, 2H), 4.03- 3.86 (m, 2H), 3.71 (d, J = 13.0 Hz, 1H), 3.31 (s, 2H), 3.06 (t, J = 12.1 Hz, 1H), 2.70 (s, 3H), 2.62 (d, J = 13.2 Hz, 1H), 2.56-2.46 (m, 1H), 2.09 (t, J = 12.5 Hz, 1H), 1.51-1.43 (m, |
| | (3R,5R)-5-(1-cyclopropyl-3-(2-fluoro- | | 1H), 1.02-0.90 (m, 5H), |
| | 4-(trifluoromethoxy)benzyl)ureido)-3- | | 0.83-0.71 (m, 2H). |
| | (hydroxymethyl)-N,3- | | |
| | imethylpiperidine-1-carboxamide | | |
| Example 99 | 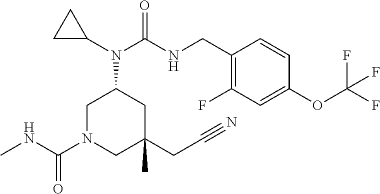 [SMILES: CNC(=O)N1C[C@H](N(C(=O)NCc2ccc(OC(F)(F)F)cc2F)C2CC2)C[C@](C)(CC#N)C1] | 486.2 | ¹H NMR (400 MHz, MeOD) δ 7.43 (t, J = 7.8 Hz, 1H), 7.08 (d, J = 8.6 Hz, 2H), 4.50-4.37 (m, 2H), 3.96-3.80 (m, 3H), 3.13 (t, J = 12.1 Hz, 1H), 2.70 (s, 3H), 2.61 (d, J = 12.8 Hz, 1H), 2.50 (d, J = 21.1 Hz, 1H), 2.46 (s, 2H), 2.12 (t, J = 12.3 Hz, 1H), 1.72 (d, J = 11.8 Hz, 1H), 1.11 (s, 3H), 0.95 (s, 2H), |
| | (3R,5R)-3-(cyanomethyl)-5-(1- | | 0.76 (s, 2H). |
| | cyclopropyl-3-(2-fluoro-4- | | |
| | (trifluoromethoxy)benzyl)ureido)-N,3- | | |
| | dimethylpiperidine-1-carboxamide | | |
| Example 100 | 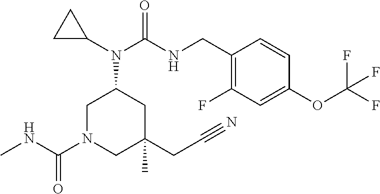 [SMILES: CNC(=O)N1C[C@H](N(C(=O)NCc2ccc(OC(F)(F)F)cc2F)C2CC2)C[C@@](C)(CC#N)C1] | 486.2 | ¹H NMR (400 MHz, MeOD) δ 7.42 (d, J = 7.9 Hz, 1H), 7.08 (d, J = 8.4 Hz, 2H), 4.49-4.36 (m, 2H), 3.86 (t, J = 12.7 Hz, 2H), 3.75 (s, 1H), 3.15 (t, J = 11.4 Hz, 1H), 2.70 (s, 3H), 2.59 (d, J = 19.0 Hz, 3H), 2.44 (d, J = 16.8 Hz, 1H), 2.13 (t, J = 13.9 Hz, 1H), 1.83 (d, J = 12.3 Hz, 1H), 1.13 (s, 3H), 0.96 (s, |
| | (3S,5R)-3-(cyanomethyl)-5-(1- | | 2H), 0.75 (s, 2H). |
| | cyclopropyl-3-(2-fluoro-4- | | |
| | (trifluoromethoxy)benzyl)ureido)-N,3- | | |
| | dimethylpiperidine-1-carboxamide | | |
| Example 101 | 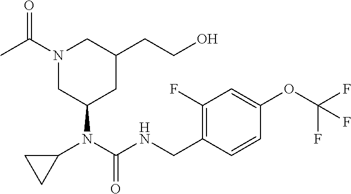 [SMILES: CC(=O)N1CC(CCO)C[C@@H](N(C(=O)NCc2ccc(OC(F)(F)F)cc2F)C2CC2)C1] | 462.2 | ¹H NMR (400 MHz, MeOD) δ 7.43 (q, J = 8.3 Hz, 1H), 7.09 (t, J = 8.5 Hz, 2H), 6.97 (dd, J = 16.0, 10.3 Hz, 1H), 4.54- 4.33 (m, 3H), 3.86 (ddd, J = 41.1, 25.4, 12.9 Hz, 2H), 3.63 (t, J = 6.4 Hz, 2H), 3.35 (d, J = 12.0 Hz, 1H), 3.25 (dd, J = 13.9, 3.1 Hz, 1H), 3.09 (dd, J = 26.7, 12.0 Hz, 1H), 2.72 (d, J = |
| | 1-((3R)-1-acetyl-5-(2- | | 13.3 Hz, 1H), 2.61-2.45 |
| | hydroxyethyl)piperidin-3-yl)-1- | | (m, 1H), 2.42-2.24 (m, |
| | cyclopropyl-3-(2-fluoro-4- | | 1H), 2.16 (d, J = 12.1 Hz, |
| | (trifluoromethoxy)benzyl)urea | | 1H), 2.10 (d, J = 8.1 Hz, |
| | | | 3H),1.94-1.76 (m, 1H), |
| | | | 1.67-1.57 (m, H), 1.55- |
| | | | 1.44 (m, 1H), 0.95 (dd, |
| | | | J = 12.6, 6.1 Hz, 2H), 0.82- |
| | | | 0.66 (m, 2H). |
| Example 102 (rac) | 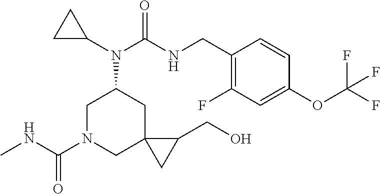 [SMILES: CNC(=O)N1C[C@H](N(C(=O)NCc2ccc(OC(F)(F)F)cc2F)C2CC2)CC2(CC2CO)C1] | 489.3 | ¹H NMR (400 MHz, MeOD) δ 7.48-7.38 (m, 1H), 7.14-7.05 (m, 2H), 7.01-6.90 (m, 1H), 4.52- 4.35 (m, 2H), 4.03-3.63 (m, 3H), 3.62-3.36 (m, 1.5H), 3.29-3.10 (m, 2H), 3.09-2.96 (m, 0.5H), 2.73- 2.66 (m, 3H), 2.66-2.40 (m, 2H), 1.58-1.38 (m, 0.5H), 1.17-0.90 (m, 3.5H), 0.82-0.52 (m, 3H), |
| | (7R)-7-(1-cyclopropyl-3-(2-fluoro-4- | | 0.51-0.21 (m, 1H). |
| | (trifluoromethoxy)benzyl)ureido)-1- | | |
| | (hydroxymethyl)-N-methyl-5- | | |
| | azaspiro[2.5]octane-5-carboxamide | | |
| Example 103 | 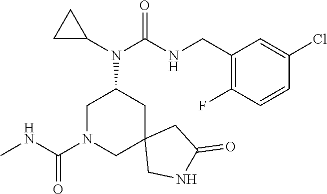 [SMILES: CNC(=O)N1C[C@H](N(C(=O)NCc2cc(Cl)ccc2F)C2CC2)CC2(CNC(=O)C2)C1] | 452.2 | ¹H NMR (400 MHz, MeOD) δ 7.36-7.22 (m, 2H), 7.13-6.96 (m, 2H), 4.41 (s, 2H), 4.03 (d, J = 13.0 Hz, 1H), 3.92 (d, J = 8.3 Hz, 1H), 3.72 (d, J = 11.9 Hz, 1H), 3.25-3.08 (m, 3H), 2.70 (s, 3H), 2.66- 2.50 (m, 2H), 2.38-2.10 (m, 3H), 1.97-1.88 (m, 1H), 0.97 (d, J = 5.4 Hz, 2H), 0.77 (s, 2H). |
| | (9R)-9-(3-(5-chloro-2-fluorobenzyl)-1- | | |
| | cyclopropylureido)-N-methyl-3-oxo- | | |
| | 2,7-diazaspiro[4.5]decane-7- | | |
| | carboxamide | | |
| Example 104 | 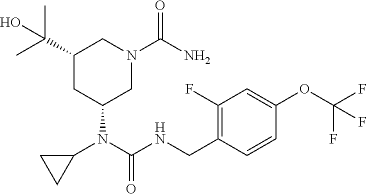 [SMILES: CC(C)(O)[C@H]1C[C@@H](N(C(=O)NCc2ccc(OC(F)(F)F)cc2F)C2CC2)CN(C(N)=O)C1] | 477.3 | ¹H NMR (400 MHz, MeOD) δ 7.44 (t, J = 8.4 Hz, 1H), 7.09 (t, J = 7.3 Hz, 2H), 6.96 (t, J = 5.7 Hz, 1H), 4.43 (s, 2H), 4.14 (d, J = 12.2 Hz, 1H), 3.87 (d, J = 11.8 Hz, 1H), 3.71-3.60 (m, 1H), 3.17 (t, J = 12.0 Hz, 1H), 2.63- 2.44 (m, 2H), 1.98 (t, J = 7.7 Hz, 2H), 1.61-1.50 (m, 1H), 1.19 (s, 6H), 0.99- |
| | (3R,5S)-3-(1-cyclopropyl-3-(2-fluoro- | | 0.88 (m, 2H), 0.86-0.68 |
| | 4-(trifluoromethoxy)benzyl)ureido)-5- | | (m, 2H). |
| | (2-hydroxypropan-2-yl)piperidine-1- | | |
| | carboxamide | | |
| Example 105 (cis) | 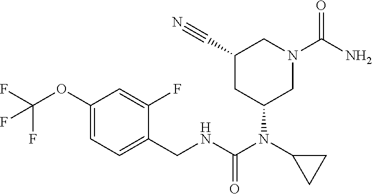 [SMILES: N#C[C@H]1C[C@@H](N(C(=O)NCc2ccc(OC(F)(F)F)cc2F)C2CC2)CN(C(N)=O)C1] | 444.1 | ¹H NMR (400 MHz, MeOD) δ 7.44 (t, J = 8.6 Hz, 1H), 7.09 (t, J = 8.4 Hz, 2H), 7.00 (s, 1H), 4.44 (s, 2H), 4.31 (d, J = 8.9 Hz, 1H), 3.89 (d, J = 12.4 Hz, 1H), 3.58 (d, J = 11.5 Hz, 1H), 3.36 (s, 1H), 2.87 (d, J = 10.9 Hz, 2H), 2.58 (s, 1H), 2.44 (d, J = 12.0 Hz, 1H), 2.27 (d, J = 11.6 Hz, 1H), 0.96 (s, 2H), |
| | (3S,5R)-3-cyano-5-(1-cyclopropyl-3-(2- | | 0.79 (s, 2H). |
| | fluoro-4- | | |
| | (trifluoromethoxy)benzyl)ureido) | | |
| | piperidine-1-carboxamide | | |
| Example 106 (cis) | 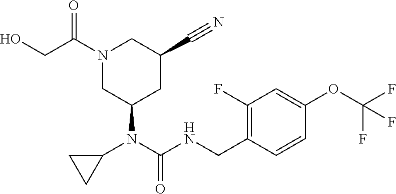 [SMILES: N#C[C@H]1C[C@@H](N(C(=O)NCc2ccc(OC(F)(F)F)cc2F)C2CC2)CN(C(=O)CO)C1] | 459.1 | ¹H NMR (400 MHz, MeOD) δ 7.44 (t, J = 8.3 Hz, 1H), 7.10 (t, J = 8.5 Hz, 2H), 7.01 (s, 1H), 4.73 (d, J = 12.3 Hz, 1H), 4.44 (s, 2H), 4.34-4.16 (m, 2H), 4.05 (d, J = 12.9 Hz, 1H), 3.68 (d, J = 11.3 Hz, 1H), 3.55-3.36 (m, 1H), 3.29-3.08 (m, 1H), 3.02-2.84 (m, 1H), 2.78 (t, J = 11.9 Hz, 1H), |
| | 1-((3R,5S)-5-cyano-1-(2- | | 2.65-2.44 (m, 2H), 2.37- |
| | hydroxyacetyl)piperidin-3-yl)-1- | | 2.21 (m, 1H), 0.97 (s, 2H), |
| | cyclopropyl-3-(2-fluoro-4- | | 0.78 (d, J = 15.4 Hz, 2H). |
| | (trifluoromethoxy)benzyl)urea | | |
| Example 107 (cis) | 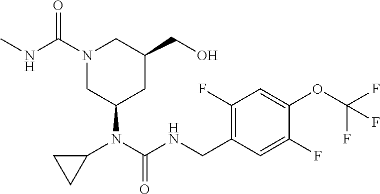 [SMILES: CNC(=O)N1C[C@@H](CO)C[C@@H](N(C(=O)NCc2cc(F)c(OC(F)(F)F)cc2F)C2CC2)C1] | 481.1 | ¹H NMR (400 MHz, CD3OD) δ 7.28 (s, 2H), 7.02 (s, 1H), 4.42 (s, 2H), 4.08 (d, J = 13.7 Hz, 1H), 3.90 (d, J = 12.2 Hz, 1H), 3.67 (s, 1H), 3.44 (s, 2H), 3.12 (t, J = 11.1 Hz, 1H), 2.70 (s, 3H), 2.55 (s, 1H), 2.40 (t, J = 12.2 Hz, 1H), 1.96-1.79 (m, 2H), 1.72 (s, 1H), 0.96 (s, 2H), 0.77 (s, 2H). |
| | (3R,5S)-3-(1-cyclopropyl-3-(2,5- | | |
| | difluoro-4- | | |
| | (trifluoromethoxy)benzyl)ureido)-5- | | |
| | (hydroxymethyl)-N-methylpiperidine- | | |
| | 1-carboxamide | | |
| Example 108 (cis) | 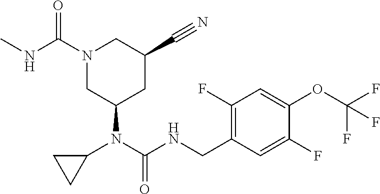 [SMILES: CNC(=O)N1C[C@@H](C#N)C[C@@H](N(C(=O)NCc2cc(F)c(OC(F)(F)F)cc2F)C2CC2)C1] | 476.2 | ¹H NMR (400 MHz, CD3OD) δ 7.37-7.19 (m, 2H), 7.08 (t, J = 5.8 Hz, 1H), 4.42 (d, J = 5.6 Hz, 2H), 4.29 (d, J = 9.4 Hz, 1H), 3.86 (d, J = 13.2 Hz, 1H), 3.62-3.52 (m, 1H), 3.27 (s, 1H), 2.91-2.79 (m, 2H), 2.71 (s, 3H), 2.62- 2.56 (m, 1H), 2.47-2.37 (m, 1H), 2.27 (d, J = 13.0 Hz, 1H), 0.98 (t, J = |
| | (3S,5R)-3-cyano-5-(1-cyclopropyl-3- | | 7.0 Hz, 2H), 0.79 (s, 2H). |
| | (2,5-difluoro-4- | | |
| | (trifluoromethoxy)benzyl)ureido)-N- | | |
| | methylpiperidine-1-carboxamide | | |
| Example 109 (cis) | 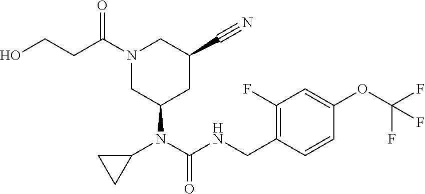 [SMILES: N#C[C@H]1C[C@@H](N(C(=O)NCc2ccc(OC(F)(F)F)cc2F)C2CC2)CN(C(=O)CCO)C1] | 473.2 | ¹H NMR (400 MHz, MeOD) δ 7.44 (d, J = 5.8 Hz, 1H), 7.10 (t, J = 8.4 Hz, 2H), 7.01 (d, J = 15.6 Hz, 1H), 4.79 (d, J = 12.3 Hz, 1H), 4.48 (d, J = 26.6 Hz, 2H), 4.29 (d, J = 13.2 Hz, 1H), 3.94 (d, J = 12.7 Hz, 1H), 3.82 (d, J = 5.4 Hz, 2H), 3.69 (d, J = 11.2 Hz,1H), 3.47 (t, J = 12.0 Hz, 1H), 3.17 (d, |
| | 1-((3R,5S)-5-cyano-1-(3- | | J = 8.1 Hz, 1H), 2.98 (s, |
| | hydroxypropanoyl)piperidin-3-yl)-1- | | 1H), 2.83 (d, J = 11.5 Hz, |
| | cyclopropyl-3-(2-fluoro-4- | | 1H), 2.77-2.66 (m, 1H), |
| | (trifluoromethoxy)benzyl)urea | | 2.63-2.44 (m, 0 H), 2.36- |
| | | | 2.21 (m, 1H), 0.98 (d, J = |
| | | | 5.5 Hz, 2H), 0.79 (d, J = |
| | | | 18.6 Hz, 2H) |
| Example 110 (cis) | 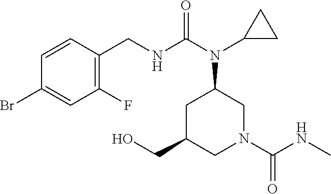 [SMILES: CNC(=O)N1C[C@@H](CO)C[C@@H](N(C(=O)NCc2ccc(Br)cc2F)C2CC2)C1] | 457.1 | ¹H NMR (400 MHz, MeOD) δ 7.27 (dd, J = 20.3, 8.6 Hz, 3H), 6.91 (s, 1H), 4.40 (d, J = 15.7 Hz, 2H), 4.07 (d, J = 12.6 Hz, 1H), 3.88 (d, J = 12.4 Hz, 1H), 3.67 (s, 1H), 3.43 (d, J = 6.6 Hz, 2H), 3.11 (t, J = 11.4 Hz, 1H), 2.70 (s, 3H), 2.52 (s, 1H), 2.39 (t, J = 12.4 Hz, 1H), 1.84 (dd, J = 22.4, 10.8 Hz, 2H), 1.70 (s, |
| | (3R,5S)-3-(3-(4-bromo-2- | | 1H), 0.94 (s, 2H), 0.75 (s, |
| | fluorobenzyl)-1-cyclopropylureido)-5- | | 2H). |
| | (hydroxymethyl)-N-methylpiperidine- | | |
| | 1-carboxamide | | |
| Example 111 (cis) | 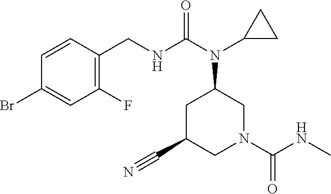 [SMILES: CNC(=O)N1C[C@@H](C#N)C[C@@H](N(C(=O)NCc2ccc(Br)cc2F)C2CC2)C1] | 452.3 | ¹H NMR (400 MHz, MeOD) δ 7.28 (dd, J = 15.2, 8.0 Hz, 3H), 6.96 (s, 1H), 4.38 (s, 2H), 4.29 (d, J = 9.6 Hz, 1H), 3.84 (d, J = 13.0 Hz, 1H), 3.56 (s, 1H), 3.26 (s, 1H), 2.85 (d, J = 7.1 Hz, 2H), 2.70 (s, 3H), 2.56 (s, 1H), 2.42 (d, J = 11.6 Hz, 1H), 2.25 (d, J = 11.5 Hz, 1H), 0.95 (s, 2H), 0.76 (s, 2H). |
| | (3R,5S)-3-(3-(4-bromo-2- | | |
| | fluorobenzyl)-1-cyclopropylureido)-5- | | |
| | cyano-N-methylpiperidine-1- | | |
| | carboxamide | | |
| Example 112 (cis) | 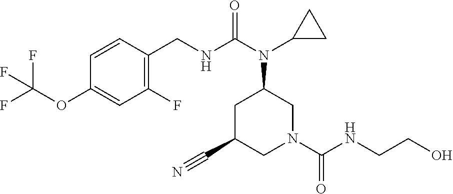 [SMILES: N#C[C@H]1C[C@@H](N(C(=O)NCc2ccc(OC(F)(F)F)cc2F)C2CC2)CN(C(=O)NCCO)C1] | 488.1 | ¹H NMR (400 MHz, CD3OD) δ 7.44 (t, J = 8.6 Hz, 1H), 7.09 (d, J = 9.0 Hz, 2H), 7.01 (s, 1H), 4.44 (s, 2H), 4.31 (d, J = 8.2 Hz, 1H), 3.90 (d, J = 11.9 Hz, 1H), 3.58 (t, J = 5.3 Hz, 3H), 3.26 (s, 3H), 2.88 (t, J = 10.6 Hz, 2H), 2.57 (s, 1H), 2.48-2.36 (m, 1H), 2.26 (d, J = 11.5 Hz, 1H), 0.96 (s, |
| | (3S,5R)-3-cyano-5-(1-cyclopropyl-3-(2- | | 2H), 0.79 (s, 2H). |
| | fluoro-4- | | |
| | (trifluoromethoxy)benzyl)ureido)-N- | | |
| | (2-hydroxyethyl)piperidine-1- | | |
| | carboxamide | | |
| Example 113 (cis) | 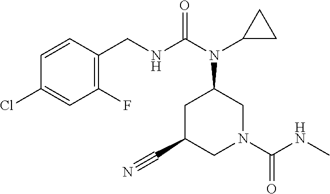 [SMILES: CNC(=O)N1C[C@@H](C#N)C[C@@H](N(C(=O)NCc2ccc(Cl)cc2F)C2CC2)C1] | 408.2 | ¹H NMR (400 MHz, MeOD) δ 7.32 (t, J = 8.3 Hz, 1H), 7.19-7.13 (m, 2H), 6.97-6.93 (m, 1H), 4.42-4.37 (m, 2H), 4.32- 4.24 (m, 1H), 3.91-3.78 (m, 1H), 3.62-3.50 (m, 1H), 3.30-3.25 (m, 1H), 2.89-2.78 (m, 2H), 2.70 (s, 3H), 2.60-2.51 (m, 1H), 2.47-2.35 (m, 1H), 2.28-2.20 (m, 1H), 1.00- |
| | (3R,5S)-3-(3-(4-chloro-2- | | 0.90 (m, 2H), 0.80-0.70 |
| | fluorobenzyl)-1-cyclopropylureido)-5- | | (m, 2H). |
| | cyano-N-methylpiperidine-1- | | |
| | carboxamide | | |
| Example 114 (cis) | 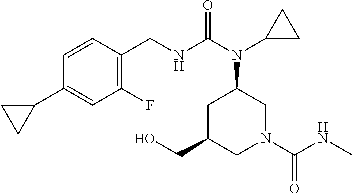 [SMILES: CNC(=O)N1C[C@@H](CO)C[C@@H](N(C(=O)NCc2ccc(C3CC3)cc2F)C2CC2)C1] | 419.2 | ¹H NMR (400 MHz, MeOD) δ 7.18 (t, J = 7.9 Hz, 1H), 6.86 (d, J = 7.9 Hz, 1H), 6.76 (d, J = 11.6 Hz, 1H), 4.45-4.28 (m, 2H), 4.08 (d, J = 13.1 Hz, 1H), 3.86 (d, J = 13.0 Hz, 1H), 3.69 (s, 1H), 3.52- 3.39 (m, 2H), 3.12 (t, J = 11.8 Hz, 1H), 2.72 (s, 3H), 2.54-2.35 (m, 2H), 1.95- 1.78 (m, 3H), 1.73 (s, 1H), |
| | (3R,5S)-3-(1-cyclopropyl-3-(4- | | 1.03-0.88 (m, 4H), 0.69 |
| | cyclopropyl-2-fluorobenzyl)ureido)-5- | | (d, J = 25.6 Hz, 4H). |
| | (hydroxymethyl)-N-methylpiperidine- | | |
| | 1-carboxamide | | |
| Example 115 (cis) | 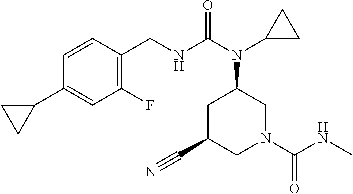 [SMILES: CNC(=O)N1C[C@@H](C#N)C[C@@H](N(C(=O)NCc2ccc(C3CC3)cc2F)C2CC2)C1] | 414.2 | ¹H NMR (400 MHz, MeOD) δ 7.18 (t, J = 8.0 Hz, 1H), 6.86 (dd, J = 7.9, 1.4 Hz, 1H), 6.80 (s, 1H), 6.76 (dd, J = 11.8, 1.5 Hz, 1H), 4.37 (d, J = 5.2 Hz, 2H), 4.29 (d, J = 9.4 Hz, 1H), 3.84 (dd, J = 12.9, 3.9 Hz, 1H), 3.56 (s, 1H), 3.26 (s, 1H), 2.91-2.76 (m, 2H), 2.70 (s, 3H), 2.60-2.49 (m, 1H), |
| | (3S,5R)-3-cyano-5-(1-cyclopropyl-3-(4- | | 2.42 (d, J = 12.1 Hz, 1H), |
| | cyclopropyl-2-fluorobenzyl)ureido)-N- | | 2.24 (d, J = 12.2 Hz, 1H), |
| | methylpiperidine-1-carboxamide | | 1.89 (dd, J = 9.1, 4.2 Hz, |
| | | | 1H), 0.96 (tt, J = 10.7, |
| | | | 5.8 Hz, 4H), 0.80-0.70 |
| | | | (m, 2H), 0.69-0.62 (m, 2H). |
| Example 116 (cis) | 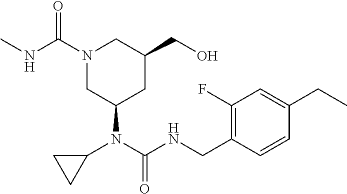 [SMILES: CCc1ccc(CNC(=O)N(C2CC2)[C@@H]2C[C@H](CO)CN(C(=O)NC)C2)c(F)c1] | 407.2 | ¹H NMR (400 MHz, CD3OD) δ 7.23 (t, J = 7.7 Hz, 1H), 6.98 (d, J = 7.6 Hz, 1H), 6.91 (d, J = 11.6 Hz, 1H), 6.80 (s, 1H), 4.39 (s, 2H), 4.09 (d, J = 13.1 Hz, 1H), 3.87 (d, J = 11.7 Hz, 1H), 3.68 (s, 1H), 3.44 (d, J = 7.0 Hz, 2H), 3.12 (t, J = 11.9 Hz, 1H), 2.70 (s, 3H), 2.63 (q, J = 7.5 Hz, 2H), 2.52 (s, 1H), |
| | (3R,5S)-3-(1-cyclopropyl-3-(4-ethyl-2- | | 2.39 (t, J = 12.5 Hz, 1H), |
| | fluorobenzyl)ureido)-5- | | 1.94-1.80 (m, 2H), 1.71 |
| | (hydroxymethyl)-N-methylpiperidine- | | (s, 1H), 1.21 (t, J = 7.6 Hz, |
| | 1-carboxamide | | 3H), 0.93 (s, 2H), 0.74 (s, |
| | | | 2H). |
| Example 117 (cis) | 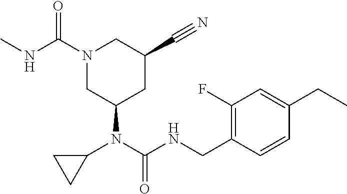 [SMILES: CCc1ccc(CNC(=O)N(C2CC2)[C@@H]2C[C@H](C#N)CN(C(=O)NC)C2)c(F)c1] | 402.3 | ¹H NMR (400 MHz, MeOD) δ 7.22 (t, J = 7.9 Hz, 1H), 6.97 (d, J = 7.6 Hz, 1H), 6.91 (d, J = 11.8 Hz, 1H), 4.39 (s, 2H), 4.29 (d, J = 9.5 Hz, 1H), 3.84 (d, J = 13.1 Hz, 1H), 3.61-3.52 (m, 1H), 3.26 (s, 1H), 2.90-2.80 (m, 2H), 2.70 (s, 3H), 2.65- 2.59 (m, 2H), 2.58-2.53 (m, 1H), 2.47-2.38 (m, |
| | (3S,5R)-3-cyano-5-(1-cyclopropyl-3-(4- | | 1H), 2.25 (d, J = 12.6 Hz, |
| | ethyl-2-fluorobenzyl)ureido)-N- | | 1H), 1.24-1.18 (m, 3H), |
| | methylpiperidine-1-carboxamide | | 0.97-0.90 (m, 2H), 0.77- |
| | | | 0.71 (m, 2H). |
| Example 118 (cis) | 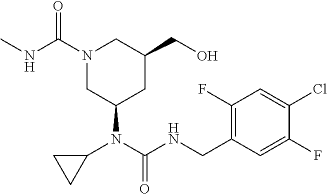 [SMILES: CNC(=O)N1C[C@@H](CO)C[C@@H](N(C(=O)NCc2cc(F)c(Cl)cc2F)C2CC2)C1] | 431.1 | ¹H NMR (400 MHz, CD3OD) δ 7.29 (s, 1H), 7.19 (s, 1H), 4.58 (s, 1H), 4.39 (s, 2H), 4.08 (d, J = 13.4 Hz, 1H), 3.89 (d, J = 11.8 Hz, 1H), 3.67 (s, 1H), 3.45 (s, 2H), 3.12 (t, J = 12.0 Hz, 1H), 2.71 (s, 3H), 2.54 (s, 1H), 2.40 (t, J = 12.0 Hz, 1H), 1.96-1.79 (m, 2H), 1.72 (s, 1H), 0.96 (s, 2H), 0.76 (s, 2H). |
| | (3R,5S)-3-(3-(4-chloro-2,5- | | |
| | difluorobenzyl)-1-cyclopropylureido)- | | |
| | 5-(hydroxymethyl)-N- | | |
| | methylpiperidine-1-carboxamide | | |
| Example 119 (cis) | 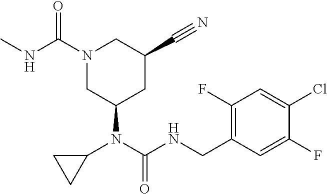 [SMILES: CNC(=O)N1C[C@@H](C#N)C[C@@H](N(C(=O)NCc2cc(F)c(Cl)cc2F)C2CC2)C1] | 426.1 | ¹H NMR (400 MHz, MeOD) δ 7.29 (dd, J = 9.2, 6.1 Hz, 1H), 7.19 (dd, J = 9.4, 6.5 Hz, 1H), 4.38 (s, 2H), 4.29 (d, J = 9.3 Hz, 1H), 3.85 (d, J = 12.9 Hz, 1H), 3.61-3.51 (m, 1H), 3.26 (s, 1H), 2.86 (dd, J = 15.7, 8.1 Hz, 2H), 2.70 (s, 3H), 2.61-2.54 (m, 1H), 2.41 (dt, J = 11.6, 8.8 Hz, 1H), 2.26 (d, J = 12.3 Hz, |
| | (3R,5S)-3-(3-(4-chloro-2,5- | | 1H), 0.96 (t, J = 7.1 Hz, |
| | difluorobenzyl)-1-cyclopropylureido)- | | 2H), 0.78 (s, 2H). |
| | 5-cyano-N-methylpiperidine-1- | | |
| | carboxamide | | |
| Example 120 | 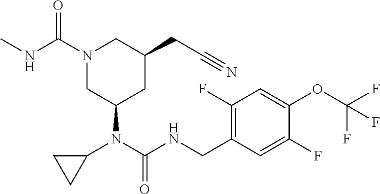 [SMILES: CNC(=O)N1C[C@@H](CC#N)C[C@@H](N(C(=O)NCc2cc(F)c(OC(F)(F)F)cc2F)C2CC2)C1] | 490.1 | ¹H NMR (400 MHz, MeOD) δ 7.35-7.22 (m, 2H), 7.05 (t, J = 5.8 Hz, 1H), 4.42 (d, J = 5.6 Hz, 2H), 4.11 (d, J = 10.8 Hz, 1H), 3.85 (d, J = 12.7 Hz, 1H), 3.74-3.61 (m, 1H), 3.21-3.12 (m, 1H), 2.70 (s, 3H), 2.60-2.41 (m, 4H), 2.08-1.87 (m, 3H), 1.03-0.92 (m, 2H), 0.84- 0.72 (m, 2H). |
| | (3R,5R)-3-(cyanomethyl)-5-(1- | | |
| | cyclopropyl-3-(2,5-difluoro-4- | | |
| | (trifluoromethoxy)benzyl)ureido)-N- | | |
| | methylpiperidine-1-carboxamide | | |
| Example 121 | 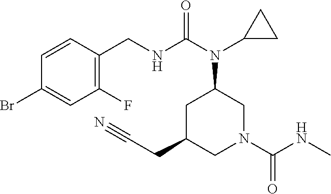 [SMILES: CNC(=O)N1C[C@@H](CC#N)C[C@@H](N(C(=O)NCc2ccc(Br)cc2F)C2CC2)C1] | 466.2 | ¹H NMR (400 MHz, MeOD) δ 7.36-7.20 (m, 3H), 4.39 (s, 2H), 4.15- 4.07 (m, 1H), 3.89-3.80 (m, 1H), 3.74-3.63 (m, 1H), 3.22-3.11 (m, 1H), 2.71 (s, 3H), 2.59-2.53 (m, 1H), 2.53-2.38 (m, 3H), 2.06-1.85 (m, 3H), 1.00-0.91 (m, 2H), 0.81- 0.71 (m, 2H). |
| | (3R,5R)-3-(3-(4-bromo-2- | | |
| | fluorobenzyl)-1-cyclopropylureido)-5- | | |
| | (cyanomethyl)-N-methylpiperidine-1- | | |
| | carboxamide | | |
| Example 122 | 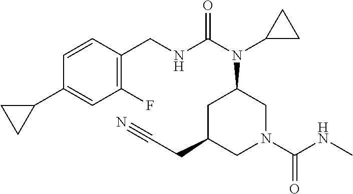 [SMILES: CNC(=O)N1C[C@@H](CC#N)C[C@@H](N(C(=O)NCc2ccc(C3CC3)cc2F)C2CC2)C1] | 428.2 | ¹H NMR (400 MHz, MeOD) δ 7.19 (t, J = 8.0 Hz, 1H), 6.86 (d, J = 7.9 Hz, 1H), 6.82- 6.78 (m, 1H), 6.75 (s, 1H), 4.43-4.29 (m, 2H), 4.12 (d, J = 13.1 Hz, 1H), 3.83 (d, J = 12.9 Hz, 1H), 3.74- 3.62 (m, 1H), 3.20-3.13 (m, 1H), 2.71 (s, 3H), 2.57- 2.40 (m, 4H), 2.07-1.85 |
| | (3R,5R)-3-(cyanomethyl)-5-(1- | | (m, 4H), 1.02-0.90 (m, |
| | cyclopropyl-3-(4-cyclopropyl-2- | | 4H), 0.79-0.62 (m, 4H). |
| | fluorobenzyl)ureido)-N- | | |
| | methylpiperidine-1-carboxamide | | |
| Example 123 | 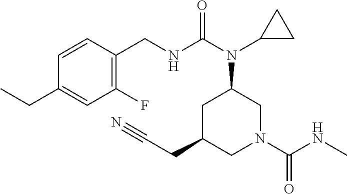 [SMILES: CCc1ccc(CNC(=O)N(C2CC2)[C@@H]2C[C@H](CC#N)CN(C(=O)NC)C2)c(F)c1] | 416.3 | ¹H NMR (400 MHz, MeOD) δ 7.23 (t, J = 7.9 Hz, 1H), 6.98 (d, J = 7.9 Hz, 1H), 6.92 (d, J = 11.6 Hz, 1H), 6.81 (t, 1H), 4.40 (s, 2H), 4.12 (d, J = 9.3 Hz, 1H), 3.84 (d, J = 12.4 Hz, 1H), 3.73-3.62 (m, 1H), 3.20-3.13 (m, 1H), 2.71 (s, 3H), 2.67-2.59 (m, 2H), 2.56-2.41 (m, 4H), |
| | (3R,5R)-3-(cyanomethyl)-5-(1- | | 2.05-1.86 (m, 3H), 1.22 |
| | cyclopropyl-3-(4-ethyl-2- | | (t, J = 7.6 Hz, 3H), 0.97- |
| | fluorobenzyl)ureido)-N- | | 0.92 (m, 2H), 0.75 (d, J = |
| | methylpiperidine-1-carboxamide | | 2.7 Hz, 2H). |
| Example 124 | 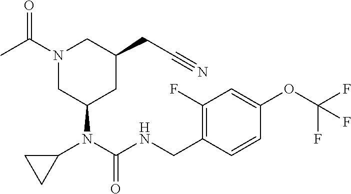 [SMILES: CC(=O)N1C[C@@H](CC#N)C[C@@H](N(C(=O)NCc2ccc(OC(F)(F)F)cc2F)C2CC2)C1] | 457.2 | ¹H NMR (400 MHz, MeOD) δ 7.47-7.39 (m, 1H), 7.09 (t, J = 8.3 Hz, 2H), 6.98 (d, J = 20.9 Hz, 1H), 4.65-4.57 (m, 1H), 4.49 (s, 1H), 4.47-4.40 (m, 2H), 3.92 (d, J = 11.8 Hz, 1H), 3.86-3.76 (m, 1H), 3.56 (s, 1H), 3.34 (d, J = 12.5 Hz, 1H), 3.08 (t, J = 12.0 Hz, 1H), 2.86-2.78 (m, 1H), 0.62- |
| | 1-((3R,5R)-1-acetyl-5- | | 2.54 (m, 1H), 2.54-2.47 |
| | (cyanomethyl)piperidin-3-yl)-1- | | (m, 2H), 2.30 (d, J = |
| | cyclopropyl-3-(2-fluoro-4- | | 12.3 Hz, 1H), 2.12 (d, |
| | (trifluoromethoxy)benzyl)urea | | J = 3.2 Hz, 3H), 2.10- |
| | | | 2.01 (m, 2H), 1.88 (s, 1H), |
| | | | 1.01-0.90 (m, 2H), 0.84- |
| | | | 0.70 (m, 2H). |
| Example 125 | 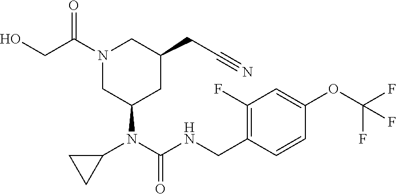 [SMILES: N#CC[C@H]1C[C@@H](N(C(=O)NCc2ccc(OC(F)(F)F)cc2F)C2CC2)CN(C(=O)CO)C1] | 473.3 | ¹H NMR (400 MHz, MeOD) δ 7.48-7.39 (m, 1H), 7.09 (t, J = 8.2 Hz, 2H), 4.59 (d, J = 13.5 Hz, 1H), 4.44 (s, 2H), 4.24 (dt, J = 24.8, 15.3 Hz, 2H), 3.79-3.58 (m, 2H), 3.17 (dd, J = 37.2, 25.1 Hz, 1H), 2.79-2.57 (m, 1H), 2.47 (ddd, J = 57.7, 15.4, 7.7 Hz, 3H), 2.16-2.01 (m, 2H), 1.99-1.83 (m, |
| | 1-((3R,5R)-5-(cyanomethyl)-1-(2- | | 1H), 0.97 (d, J = 6.3 Hz, |
| | hydroxyacetyl)piperidin-3-yl)-1- | | 2H), 0.78 (dd, J = 17.2, |
| | cyclopropyl-3-(2-fluoro-4- | | 7.3 Hz, 2H). |
| | (trifluoromethoxy)benzyl)urea | | |
| Example 126 | 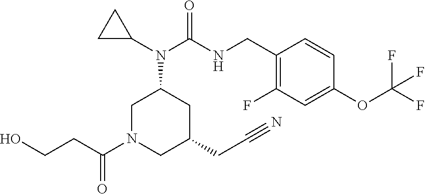 [SMILES: N#CC[C@H]1C[C@@H](N(C(=O)NCc2ccc(OC(F)(F)F)cc2F)C2CC2)CN(C(=O)CCO)C1] | 487.3 | ¹H NMR (400 MHz, MeOD) δ 7.47-7.39 (m, 1H), 7.09 (t, J = 8.5 Hz, 2H), 7.02-6.82 (m, 1H), 4.67-4.51 (m, 1H), 4.44 (d, J = 3.3 Hz, 2H), 4.06- 3.91 (m, 1H), 3.87-3.72 (m, 3H), 3.60-3.51 (m, 0.5H), 3.36 (d, J = 12.7 Hz, 0.5H), 3.14-3.06 (m, 0.5H), 2.86-2.74 (m, 0.5H), 2.71-2.59 (m, 2H), |
| | 1-((3R,5R)-5-(cyanomethyl)-1-(3- | | 2.56-2.46 (m, 3H), 2.32 |
| | hydroxypropanoyl)piperidin-3-yl)-1- | | (t, J = 12.2 Hz, 0.5H), 2.10- |
| | cyclopropyl-3-(2-fluoro-4- | | 2.00 (m, 2H), 1.89 (s, |
| | (trifluoromethoxy)benzyl)urea | | 0.5H), 1.01-0.90 (m, 2H), |
| | | | 0.77 (d, J = 17.5 Hz, 2H). |
| Example 127 | 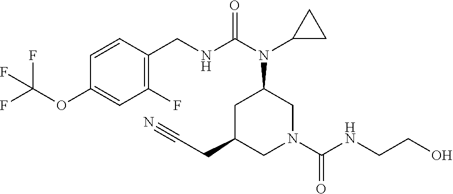 [SMILES: N#CC[C@H]1C[C@@H](N(C(=O)NCc2ccc(OC(F)(F)F)cc2F)C2CC2)CN(C(=O)NCCO)C1] | 502.3 | ¹H NMR (400 MHz, MeOD) δ 7.44 (t, J = 8.6 Hz, 1H), 7.09 (t, J = 7.4 Hz, 2H), 6.99 (t, J = 5.9 Hz, 1H), 4.44 (d, J = 5.4 Hz, 2H), 4.12 (d, J = 10.7 Hz, 1H), 3.88 (d, J = 12.8 Hz, 1H), 3.70 (dd, J = 13.4, 9.2 Hz, 1H), 3.57 (t, J = 5.8 Hz, 2H), 3.26 (t, J = 5.8 Hz, |
| | (3R,5R)-3-(cyanomethyl)-5-(1- | | 2H), 3.20-3.13 (m, 1H), |
| | cyclopropyl-3-(2-fluoro-4- | | 2.58-2.53 (m, 1H), 2.52- |
| | (trifluoromethoxy)benzyl)ureido)-N- | | 2.40 (m, 3H), 2.07- |
| | (2-hydroxyethyl)piperidine-1- | | 1.87 (m, 3H), 0.96 (d, |
| | carboxamide | | J = 3.7 Hz, 2H), 0.81- |
| | | | 0.72 (m, 0H). |
| Example 128 | 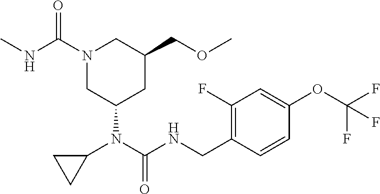 [SMILES: CNC(=O)N1C[C@@H](COC)C[C@H](N(C(=O)NCc2ccc(OC(F)(F)F)cc2F)C2CC2)C1] | 477.3 | ¹H NMR (400 MHz, MeOD) δ 7.43 (t, J = 8.6 Hz, 1H), 7.09 (t, J = 8.0 Hz, 2H), 6.94 (t, J = 5.8 Hz, 1H), 4.43 (t, J = 5.7 Hz, 2H), 3.98-3.86 (m, 2H), 3.77 (ddd, J = 11.9, 7.8, 4.3 Hz, 1H), 3.42-3.32 (m, 5H), 3.17 (t, J = 11.9 Hz, 1H), 2.89 (dd, J = |
| | (3S,5S)-3-(1-cyclopropyl-3-(2-fluoro-4- | | 13.7, 3.3 Hz, 1H), |
| | (trifluoromethoxy)benzyl)ureido)-5- | | 2.71 (d, J = 4.0 Hz, |
| | (methoxymethyl)-N-methylpiperidine- | | 3H), 2.55-2.46 (m, |
| | 1-carboxamide | | 1H), 2.27 (td, J = 12.8, |
| | | | 5.4 Hz, 1H), 2.13 (s, |
| | | | 1H), 1.84 (d, J = |
| | | | 11.6 Hz, 1H), 0.99- |
| | | | 0.90 (m, 2H), 0.75 |
| | | | (dd, J = 6.6, 3.9 Hz, |
| | | | 2H). |
| Example 129 | 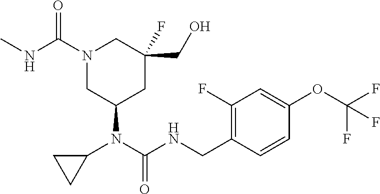 [SMILES: CNC(=O)N1C[C@H](N(C(=O)NCc2ccc(OC(F)(F)F)cc2F)C2CC2)C[C@](F)(CO)C1] | 481.3 | ¹H NMR (400 MHz, MeOD) δ 7.43 (t, J = 8.6 Hz, 1H), 7.13-7.02 (m, 2H), 6.97 (t, J = 5.8 Hz, 1H), 4.51-4.37 (m, 2H), 4.24-4.09 (m, 1H), 4.01- 3.81 (m, 2H), 3.66-3.45 (m, 2H), 3.29-3.21 (m, 1H), 2.94-2.76 (m, 1H), 2.71 (s, 3H), 2.63-2.55 (m, 1H), 2.45-2.24 (m, 1H), 2.07-1.95 (m, 1H), |
| | (3R,5R)-5-(1-cyclopropyl-3-(2-fluoro- | | 1.00-0.88 (m, 2H), 0.84- |
| | 4-(trifluoromethoxy)benzyl)ureido)-3- | | 0.68 (m, 2H). |
| | fluoro-3-(hydroxymethyl)-N- | | |
| | methylpiperidine-1-carboxamide | | |
| Example 130 (cis) | 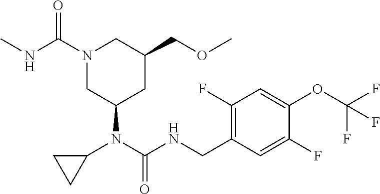 [SMILES: CNC(=O)N1C[C@@H](COC)C[C@@H](N(C(=O)NCc2cc(F)c(OC(F)(F)F)cc2F)C2CC2)C1] | 495.5 | ¹H NMR (400 MHz, MeOD) δ 7.30-7.23 (m, 2H), 7.05-6.99 (m, 1H), 4.58 (s, 1H), 4.45-4.37 (m, 2H), 4.05 (d, J = 14.0 Hz, 1H), 3.88 (d, J = 12.2 Hz, 1H), 3.70- 3.59 (m, 1H), 3.32 (s, 3H), 3.29-3.23 (m, 2H), 3.12 (t, J = 11.9 Hz, 1H), 2.70 (s, 3H), 2.58-2.50 (m, 1H), 2.45-2.36 (m, |
| | (3R,5S)-3-(1-cyclopropyl-3-(2,5- | | 1H), 1.91-1.78 (m, 3H), |
| | difluoro-4- | | 0.99-0.91 (m, 2H), 0.79- |
| | (trifluoromethoxy)benzyl)ureido)-5- | | 0.72 (m, 2H). |
| | (methoxymethyl)-N-methylpiperidine- | | |
| Example 131 (cis) | 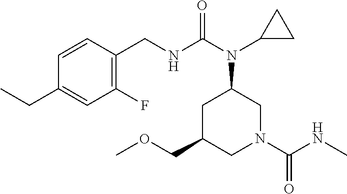 [SMILES: CCc1ccc(CNC(=O)N(C2CC2)[C@@H]2C[C@H](COC)CN(C(=O)NC)C2)c(F)c1] | 421.3 | ¹H NMR (400 MHz, MeOD) δ 14.57-14.51 (m, 1H), 7.22 (t, J = 7.9 Hz, 1H), 6.97 (d, J = 7.7 Hz, 1H), 6.91 (d, J = 11.5 Hz, 1H), 6.77 (s, 1H), 4.39 (d, J = 3.1 Hz, 2H), 4.06 (d, J = 13.1 Hz, 1H), 3.86 (d, J = 12.9 Hz, 1H), 3.70-3.61 (m, 1H), 3.32 (s, 3H), 3.27 (d, J = |
| | (3R,5S)-3-(1-cyclopropyl-3-(4-ethyl-2- | | 6.4 Hz, 2H), 3.11 (s, 1H), |
| | fluorobenzyl)ureido)-5- | | 2.70 (s, 3H), 2.63 (q, |
| | (methoxymethyl)-N-methylpiperidine- | | J = 7.6 Hz, 2H), 2.51 (s, |
| | 1-carboxamide | | 1H), 2.44-2.35 (m, 1H), |
| | | | 1.86 (d, J = 8.0 Hz, 3H), |
| | | | 1.21 (t, J = 7.6 Hz, 3H), |
| | | | 0.94-0.88 (m, 2H), 0.75- |
| | | | 0.69 (m, 2H). |
| Example 132 (cis) | 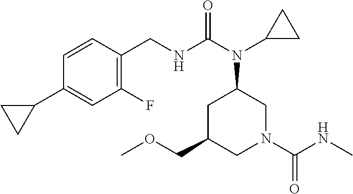 [SMILES: CNC(=O)N1C[C@@H](COC)C[C@@H](N(C(=O)NCc2ccc(C3CC3)cc2F)C2CC2)C1] | 433.3 | ¹H NMR (400 MHz, MeOD) δ 7.18 (t, J = 8.0 Hz, 1H), 6.86 (d, J = 7.9 Hz, 1H), 6.77 (s, 1H), 6.74 (s, 1H), 4.37 (d, J = 4.2 Hz, 2H), 4.06 (d, J = 13.1 Hz, 1H), 3.86 (d, J = 12.6 Hz, 1H), 3.65 (dd, J = 7.7, 3.6 Hz, 1H), 3.32 (s, 3H), 3.26 (dd, J = 13.5, 7.0 Hz, 2H), 3.10 (t, J = 12.0 Hz, 1H), |
| | (3R,5S)-3-(1-cyclopropyl-3-(4- | | 2.70 (s, 3H), 2.50 (dt, J = |
| | cyclopropyl-2-fluorobenzyl)ureido)-5- | | 10.3, 3.4 Hz, 1H), 2.45- |
| | (methoxymethyl)-N-methylpiperidine- | | 2.32 (m, 1H), 1.94-1.75 |
| | 1-carboxamide | | (m, 4H), 1.01-0.94 (m, |
| | | | 2H), 0.92 (dd, J = 8.1, |
| | | | 4.2 Hz, 2H), 0.77-0.69 (m, |
| | | | 2H), 0.69-0.60 (m, 2H). |
| Example 133 (cis) | 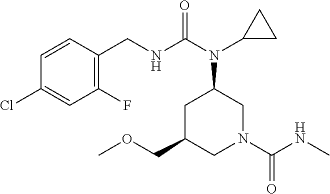 [SMILES: CNC(=O)N1C[C@@H](COC)C[C@@H](N(C(=O)NCc2ccc(Cl)cc2F)C2CC2)C1] | 427.2 | ¹H NMR (400 MHz, MeOD) δ 7.32 (t, J = 8.3 Hz, 1H), 7.19-7.12 (m, 2H), 6.90 (t, J = 5.7 Hz, 1H), 4.39 (s, 2H), 4.05 (d, J = 12.3 Hz, 1H), 3.87 (d, J = 13.1 Hz, 1H), 3.70-3.60 (m, 1H), 3.32 (s, 3H), 3.28- 3.23 (m, 2H), 3.11 (t, J = 12.0 Hz, 1H), |
| | (3R,5S)-3-(3-(4-chloro-2- | | 2.70 (s, 3H), 2.52 (dt, |
| | fluorobenzyl)-1-cyclopropylureido)-5- | | J = 10.4, 3.5 Hz, 1H), |
| | (methoxymethyl)-N-methylpiperidine- | | 2.44-2.34 (m, 1H), |
| | 1-carboxamide | | 1.90-1.78 (m, 3H), |
| | | | 0.93 (d, J = 3.4 Hz, |
| | | | 2H), 0.74 (s, 2H). |
| Example 134 | 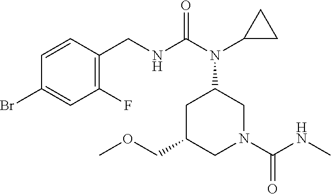 | 471.2 | ¹H NMR (400 MHz, MeOD) δ 7.36-7.19 (m, 3H), 6.92 (t, J = 5.9 Hz, 1H), 4.38 (s, 2H), 4.05 (d, J = 12.0 Hz, 1H), 3.92-3.83 (m, 1H), 3.72-3.58 (m, 1H), 3.32 (d, J = 3.1 Hz, 3H), 3.27 (t, J = 5.7 Hz, 2H), 3.15- 3.06 (m, 1H), 2.69 (s, 3H), 2.56-2.48 (m, 1H), 2.40 (dd, J = 13.1, 10.9 Hz, |
| | (3S,5R)-3-(3-(4-bromo-2- | | 1H), 1.94-1.75 (m, 3H), |
| | fluorobenzyl)-1-cyclopropylureido)-5- | | 0.98-0.89 (m, 2H), 0.74 |
| | (methoxymethyl)-N-methylpiperidine- | | (dd, J = 5.9, 3.7 Hz, 2H). |
| | 1-carboxamide | | |
| Example 135 (cis) | 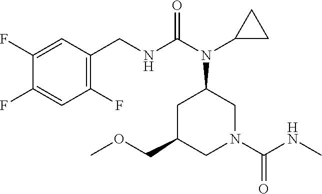 [SMILES: CNC(=O)N1C[C@@H](COC)C[C@@H](N(C(=O)NCc2cc(F)c(F)cc2F)C2CC2)C1] | 429.3 | ¹H NMR (400 MHz, MeOD) δ 7.22 (d, J = 8.7 Hz, 1H), 7.16-7.08 (m, 1H), 4.37 (s, 2H), 4.05 (d, J = 12.7 Hz, 1H), 3.88 (d, J = 12.5 Hz, 1H), 3.65 (d, J = 3.8 Hz, 1H), 3.32 (s, 3H), 3.27 (d, J = 6.2 Hz, 2H), 3.12 (t, J = 11.9 Hz, 1H), 2.70 (s, 3H), 2.56-2.50 |
| | (3R,5S)-3-(1-cyclopropyl-3-(2,4,5- | | (m, 1H), 2.45-2.36 (m, |
| | trifluorobenzyl)ureido)-5- | | 1H), 1.86 (d, J = 8.0 Hz, |
| | (methoxymethyl)-N-methylpiperidine- | | 3H), 0.94 (d, J = 3.3 Hz, |
| | 1-carboxamide | | 2H), 0.74 (s, 2H). |
| Example 136 (cis) | 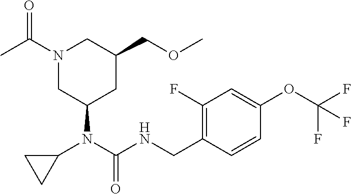 [SMILES: COC[C@H]1C[C@@H](N(C(=O)NCc2ccc(OC(F)(F)F)cc2F)C2CC2)CN(C(C)=O)C1] | 462.3 | ¹H NMR (400 MHz, MeOD) δ 7.47-7.39 (m, 1H), 7.12-7.04 (m, 2H), 4.61-4.56 (m, 1H), 4.49- 4.38 (m, 3H), 3.93 (d, J = 13.2 Hz, 1H), 3.86-3.75 (m, 1H), 3.58-3.47 (m, 1H), 3.33 (d, J = 2.9 Hz, 3H), 3.30-3.23 (m, 3H), 3.06 (t, J = 11.9 Hz, 1H), 2.80-2.70 (m, 1H), 2.60- 2.48 (m, 1H), 2.26 (t, 1H), |
| | 1-((3R,5S)-1-acetyl-5- | | 2.10 (d, J = 2.1 Hz, 3H), |
| | (methoxymethyl)piperidin-3-yl)-1- | | 1.99-1.93 (m, 1H), 1.92- |
| | cyclopropyl-3-(2-fluoro-4- | | 1.73 (m, 2H), 0.99-0.88 |
| | (trifluoromethoxy)benzyl)urea | | (m, 2H), 0.82-0.67 (m, |
| | | | 2H). |
| Example 137 | 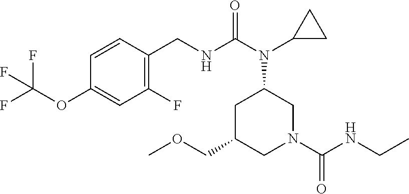 [SMILES: CCNC(=O)N1C[C@H](COC)C[C@H](N(C(=O)NCc2ccc(OC(F)(F)F)cc2F)C2CC2)C1] | 491.3 | ¹H NMR (400 MHz, MeOD) δ 7.43 (t, J = 8.6 Hz, 1H), 7.09 (t, J = 7.8 Hz, 2H), 4.43 (s, 2H), 4.07 (d, J = 13.2 Hz, 1H), 3.89 (d, J = 12.4 Hz, 1H), 3.66 (s, 1H), 3.32 (s, 3H), 3.29-3.24 (m, 2H), 3.14 (dt, J = 26.0, 9.5 Hz, 3H), 2.58-2.46 (m, 1H), 2.45-2.33 |
| | (3S,5R)-3-(1-cyclopropyl-3-(2-fluoro- | | (m, 1H), 1.86 (d, J = |
| | 4-(trifluoromethoxy)benzyl)ureido)-N- | | 8.5 Hz, 3H), 1.09 (t, |
| | ethyl-5-(methoxymethyl)piperidine-1- | | J = 7.2 Hz, 3H), 0.94 |
| | carboxamide | | (d, J = 3.7 Hz, 2H), |
| | | | 0.76 (s, 2H). |
| Example 138 | 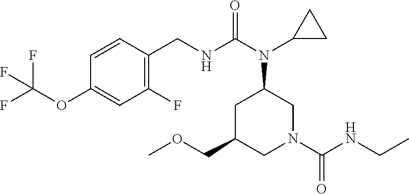 [SMILES: CCNC(=O)N1C[C@@H](COC)C[C@@H](N(C(=O)NCc2ccc(OC(F)(F)F)cc2F)C2CC2)C1] | 491.3 | ¹H NMR (400 MHz, MeOD) δ 7.43 (t, J = 8.6 Hz, 1H), 7.09 (t, J = 7.8 Hz, 2H), 4.49- 4.35 (m, 2H), 4.07 (d, J = 13.5 Hz, 1H), 3.89 (d, J = 12.4 Hz, 1H), 3.66 (s, 1H), 3.32 (s, 3H), 3.29-3.24 (m, 2H), 3.16 (q, J = 7.3 Hz, 2H), 3.08 (d, J = 11.9 Hz, 1H), 2.59-2.48 (m, 1H), |
| | (3R,5S)-3-(1-cyclopropyl-3-(2-fluoro- | | 2.44-2.31 (m, 1H), 1.86 |
| | 4-(trifluoromethoxy)benzyl)ureido)-N- | | (d, J = 8.5 Hz, 3H), 1.09 |
| | ethyl-5-(methoxymethyl)piperidine-1- | | (t, J = 7.2 Hz, 3H), 0.94 |
| | carboxamide | | (d, J = 3.8 Hz, 2H), 0.81- |
| | | | 0.70 (m, 2H). |
| Example 139 | 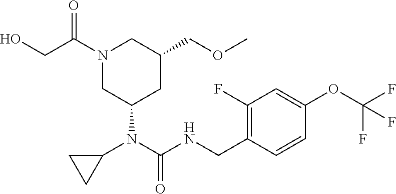 [SMILES: COC[C@@H]1C[C@H](N(C(=O)NCc2ccc(OC(F)(F)F)cc2F)C2CC2)CN(C(=O)CO)C1] | 478.3 | ¹H NMR (400 MHz, MeOD) δ 7.43 (dd, J = 13.7, 8.7 Hz, 1H), 7.09 (t, J = 8.3 Hz, 2H), 4.56 (d, J = 9.1 Hz, 1H), 4.43 (s, 2H), 4.30-4.15 (m, 2H), 3.73 (d, J = 11.7 Hz, 1H), 3.67- 3.55 (m, 1H), 3.30 (s, 3H), 3.30-3.25 (m, 2H), 3.22 (s, 1H), 3.12 (s, 1H), 2.68 (s, 1H), 2.60-2.49 (m, 1H), 2.31 (d, J = 12.4 Hz, |
| | 1-cyclopropyl-3-(2-fluoro-4- | | 1H), 2.01-1.78 (m, 3H), |
| | (trifluoromethoxy)benzyl)-1-((3S,5R)- | | 0.93 (dd, J = 15.6, 9.3 Hz, |
| | 1-(2-hydroxyacetyl)-5- | | 2H), 0.76 (dd, J = 19.1, |
| | (methoxymethyl)piperidin-3-yl)urea | | 9.8 Hz, 2H). |
| Example 140 | 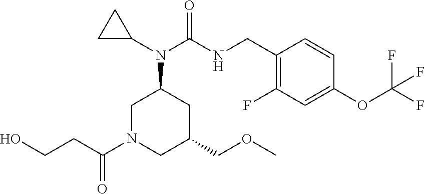 [SMILES: COC[C@H]1C[C@H](N(C(=O)NCc2ccc(OC(F)(F)F)cc2F)C2CC2)CN(C(=O)CCO)C1] | 492.1 | ¹H NMR (400 MHz, MeOD) δ 7.48-7.38 (m, 1H), 7.16-7.03 (m, 2H), 4.66-4.47 (m, 1H), 4.47- 4.38 (m, 2H), 4.11-3.88 (m, 1H), 3.87-3.79 (m, 2H), 3.78-3.51 (m, 1H), 3.33 (d, J = 3.2 Hz, 3H), 3.30-3.00 (m, 3H), 2.79- 2.70 (m, 0.5H), 2.69-2.49 (m, 3H), 2.35-2.22 (m, 0.5H), 2.05-1.76 (m, 3H), |
| | 1-cyclopropyl-3-(2-fluoro-4- | | 1.01-0.88 (m, 2H), 0.83- |
| | (trifluoromethoxy)benzyl)-1-((3R,5S)- | | 0.69 (m, 2H). |
| | 1-(3-hydroxypropanoyl)-5- | | |
| | (methoxymethyl)piperidin-3-yl)urea | | |
| Example 141 (cis) | 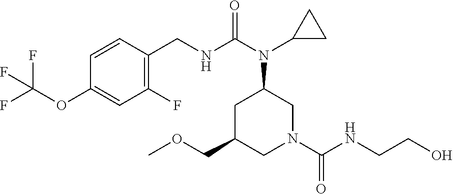 [SMILES: COC[C@H]1C[C@@H](N(C(=O)NCc2ccc(OC(F)(F)F)cc2F)C2CC2)CN(C(=O)NCCO)C1] | 507.3 | ¹H NMR (400 MHz, CD3OD) δ 7.44 (t, J = 8.6 Hz, 1H), 7.09 (t, J = 7.5 Hz, 2H), 4.44 (d, J = 2.4 Hz, 2H), 4.08 (d, J = 13.4 Hz, 1H), 3.91 (d, J = 12.4 Hz, 1H), 3.69 (s, 1H), 3.57 (t, J = 5.8 Hz, 2H), 3.33 (s, 3H), 3.30-3.28 (m, 2H), 0.26-3.24 (m, 2H) 3.16- 3.08 (m, 1H), 2.58-2.50 (m, 1H), 2.46-2.37 (m, |
| | (3R,5S)-3-(1-cyclopropyl-3-(2-fluoro- | | 1H), 1.92-1.83 (m, 3H), |
| | 4-(trifluoromethoxy)benzyl)ureido)-N- | | 0.98-0.91 (m, 2H), 0.79- |
| | (2-hydroxyethyl)-5- | | 0.73 (m, 2H). |
| | (methoxymethyl)piperidine-1- | | |
| | carboxamide | | |
| Example 143 (cis) | 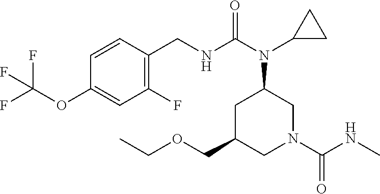 [SMILES: CCOC[C@H]1C[C@@H](N(C(=O)NCc2ccc(OC(F)(F)F)cc2F)C2CC2)CN(C(=O)NC)C1] | 491.2 | ¹H NMR (400 MHz, CD3OD) δ 7.43 (t, J = 8.6 Hz, 1H), 7.09 (t, J = 7.7 Hz, 2H), 4.44 (s, 2H), 4.07 (d, J = 13.2 Hz, 1H), 3.88 (d, J = 12.6 Hz, 1H), 3.66 (d, J = 4.6 Hz, 1H), 3.52-3.44 (m, 2H), 3.37- 3.32 (m, 2H), 3.12 (t, J = 12.0 Hz, 1H), 2.70 (s, 3H), 2.58-2.50 (m, 1H), 2.46-2.36 (m, 1H), 1.91- |
| | (3R,5S)-3-(1-cyclopropyl-3-(2-fluoro- | | 1.77 (m, 3H), 1.18 (t, J = |
| | 4-(trifluoromethoxy)benzyl)ureido)-5- | | 7.0 Hz , 0H), 0.95 (d, J = |
| | (ethoxymethyl)-N-methylpiperidine-1- | | 3.4 Hz, 2H), 0.76 (d, J = |
| | carboxamide | | 2.8 Hz, 0H). |
| Example 144 (cis) | 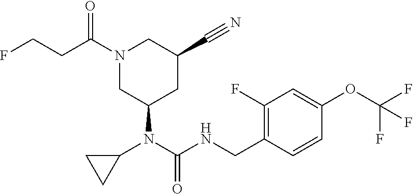 [SMILES: N#C[C@H]1C[C@@H](N(C(=O)NCc2ccc(OC(F)(F)F)cc2F)C2CC2)CN(C(=O)CCF)C1] | 475.4 | ¹H NMR (400 MHz, CD3OD) δ 7.53-7.35 (m, 1H), 7.09 (t, J = 8.3 Hz, 2H), 7.00 (d, J = 15.7 Hz, 1H), 4.75 (d, J = 5.1 Hz, 2H), 4.67-4.57 (m, 1H), 4.52-4.39 (m, 3H), 4.24 (d, J = 12.2 Hz, 1H), 3.90 (d, J = 12.5 Hz, 1H), 3.70 (s, 1H), 3.50-3.39 (m, 1H), 3.24-3.09 (m, 1H), 3.02-2.71 (m, 3H), 2.63- |
| | 1-((3R,5S)-5-cyano-1-(3- | | 2.45 (m, 2H), 2.30 (d, J = |
| | fluoropropanoyl)piperidin-3-yl)-1- | | 29.7 Hz, 1H), 0.98 (d, J = |
| | cyclopropyl-3-(2-fluoro-4- | | 6.2 Hz, 2H), 0.78 (d, J = |
| | (trifluoromethoxy)benzyl)urea | | 23.2 Hz, 2H). |
| Example 145 | 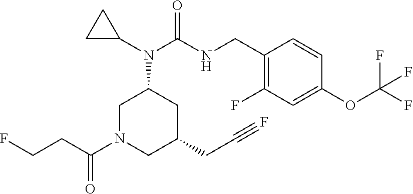 | 489.2 | ¹H NMR (400 MHz, CD3OD) δ 7.55-7.36 (m, 1H), 7.09 (t, J = 8.5 Hz, 2H), 4.79-4.73 (m, 1H), 4.68-4.62 (m, 2H), 4.55- 4.39 (m, 3H), 4.03-3.87 (m, 1H), 3.82-3.74 (m, 1H), 3.63-3.49 (m, 1H), 3.36 (d, J = 12.4 Hz, 1H), 3.17-3.08 (m, 1H), 2.97- 2.72 (m, 3H), 2.63-2.44 (m, 3H), 2.35 (t, J = |
| | 1-((3R,5R)-5-(cyanomethyl)-1-(3- | | 12.3 Hz, 1H), 2.15-1.87 |
| | fluoropropanoyl)piperidin-3-yl)-1- | | (m, 3H), 1.02-0.90 (m, |
| | cyclopropyl-3-(2-fluoro-4- | | 2H), 0.86-0.68 (m, 2H). |
| | (trifluoromethoxy)benzyl)urea | | |
| Example 146 | 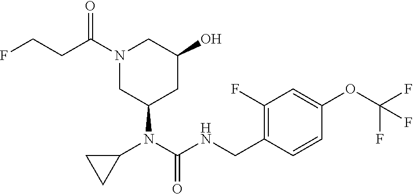 [SMILES: O=C(CCF)N1C[C@@H](O)C[C@@H](N(C(=O)NCc2ccc(OC(F)(F)F)cc2F)C2CC2)C1] | 466.2 | ¹H NMR (400 MHz, MeOD) δ 7.48-7.37 (m, 1H), 7.15-7.02 (m, 2H), 6.99-6.86 (m, 1H), 4.79- 4.71 (m, 1H), 4.67-4.59 (m, 1H), 4.57-4.37 (m, 3H), 4.14-4.05 (m, 1H), 3.92-3.77 (m, 1.5H), 3.46- 3.32 (m, 0.5H), 3.29- 3.12 (m, 1.5H), 2.95-2.67 (m, 2.5H), 2.64-2.51 (m, 1H), 2.51-2.33 (m, 1H), |
| | 1-cyclopropyl-3-(2-fluoro-4- | | 2.00-1.84 (m, 1H), 0.98- |
| | (trifluoromethoxy)benzyl)-1-((3R,5R)- | | 0.89 (m, 2H), 0.84-0.67 |
| | 1-(3-fluoropropanoyl)-5- | | (m, 2H). |
| | hydroxypiperidin-3-yl)urea | | |
| Example 147 (rac) | 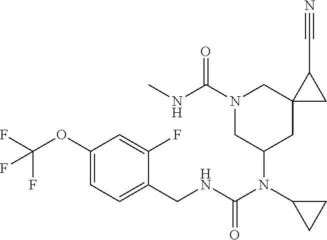 [SMILES: CNC(=O)N1CC(N(C(=O)NCc2ccc(OC(F)(F)F)cc2F)C2CC2)CC2(CC2C#N)C1] | 484.3 | ¹H NMR (400 MHz, MeOD) δ 7.48-7.37 (m, 1H), 7.17-7.04 (m, 2H), 7.04-6.93 (m, 1H), 4.53- 4.34 (m, 2H), 4.11-3.77 (m, 2H), 3.66-3.42 (m, 1H), 3.21-2.86 (m, 2H), 2.74-2.68 (m, 3H), 2.67- 2.59 (m, 1H), 2.58-2.45 (m, 1H), 1.75-1.58 (m, 1H), 1.56-1.44 (m, 0.5H), 1.34-1.23 (m, 1H), 1.21- 1.11 (m, 1.5H), 1.01-0.91 (m, 2H), 0.90-0.70 (m, 2H). |
| | 1-cyano-7-(1-cyclopropyl-3-(2-fluoro- | | |
| | 4-(trifluoromethoxy)benzyl)ureido)-N- | | |
| | methyl-5-azaspiro[2.5]octane-5- | | |
| | carboxamide | | |
| Example 148 | 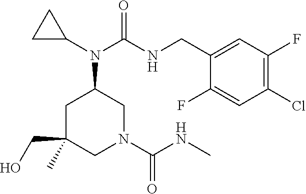 [SMILES: CNC(=O)N1C[C@H](N(C(=O)NCc2cc(F)c(Cl)cc2F)C2CC2)C[C@](C)(CO)C1] | 445.2 | ¹H NMR (400 MHz, MeOD) δ 7.34-7.23 (m, 1H), 7.22-7.14 (m, 1H), 6.99 (t, J = 5.6 Hz, 1H), 4.44-4.33 (m, 2H), 3.96- 3.87 (m, 1H), 3.85-3.75 (m, 2H), 3.44-3.32 (m, 2H), 3.13 (t, J = 12.0 Hz, 1H), 2.70 (s, 3H), 2.56- 2.45 (m, 2H), 1.92 (t, J = 12.8 Hz, 1H), 1.83-1.73 (m, 1H), 0.99-0.91 (m, |
| | (3S,5R)-5-(3-(4-chloro-2,5- | | 5H), 0.79-0.70 (m, 2H). |
| | difluorobenzyl)-1-cyclopropylureido)- | | |
| | 3-(hydroxymethyl)-N,3- | | |
| | dimethylpiperidine-1-carboxamide | | |
| Example 149 | 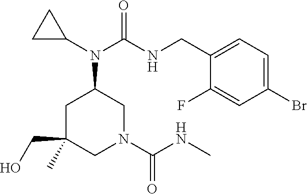 [SMILES: CNC(=O)N1C[C@H](N(C(=O)NCc2ccc(Br)cc2F)C2CC2)C[C@](C)(CO)C1] | 471.2 | ¹H NMR (400 MHz, MeOD) δ 7.36-7.21 (m, 3H), 6.91 (t, J = 5.8 Hz, 1H), 4.45-4.30 (m, 2H), 3.94-3.75 (m, 3H), 3.45- 3.33 (m, 2H), 3.12 (t, J = 12.0 Hz, 1H), 2.69 (s, 3H), 2.55-2.43 (m, 2H), 1.96- 1.84 (m, 1H), 1.81-1.72 (m, 1H), 0.98-0.89 (m, 5H), 0.79-0.68 (m, 2H). |
| | (3S,5R)-5-(3-(4-bromo-2- | | |
| | fluorobenzyl)-1-cyclopropylureido)-3- | | |
| | (hydroxymethyl)-N,3- | | |
| | dimethylpiperidine-1-carboxamide | | |
| Example 150 | 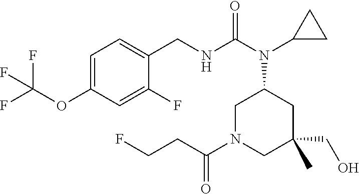 [SMILES: C[C@]1(CO)C[C@@H](N(C(=O)NCc2ccc(OC(F)(F)F)cc2F)C2CC2)CN(C(=O)CCF)C1] | 494.3 | ¹H NMR (400 MHz, MeOD) δ 7.43 (q, J = 8.9 Hz, 1H), 7.15-7.04 (m, 2H), 7.01-6.87 (m, 1H), 4.75 (q, 1H), 4.63 (q, J = 5.6 Hz, 1H), 4.54-4.47 (m,0.5H), 4.47-4.38 (m, 2H), 4.28 (d, J = 13.4 Hz, 0.5H), 3.94-3.81 (m, 1.5H), 3.75-3.63 (m, 0.5H), 3.51-3.45 (m, 0.5H), 3.38-3.32 (m, 2H), |
| | 1-cyclopropyl-3-(2-fluoro-4- | | 3.07 (t, J = 12.0 Hz, 0.5H), |
| | (trifluoromethoxy)benzyl)-1-((3R,5S)- | | 3.01-2.70 (m, 2.5H), 2.58- |
| | 1-(3-fluoropropanoyl)-5- | | 2.47 (m, 1H), 2.40 (d, J = |
| | (hydroxymethyl)-5-methylpiperidin-3- | | 13.3 Hz, 0.5H), 2.08-1.87 |
| | yl)urea | | (m, 1.5H), 1.74-1.63 (m, |
| | | | 0.5H), 1.02-0.90 (m, 5H), |
| | | | 0.86-0.66 (m, 2H). |
| Example 151 | 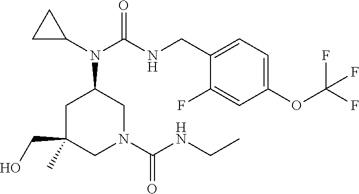 [SMILES: CCNC(=O)N1C[C@H](N(C(=O)NCc2ccc(OC(F)(F)F)cc2F)C2CC2)C[C@](C)(CO)C1] | 491.5 | ¹H NMR (400 MHz, MeOD) δ 7.43 (t, J = 8.6 Hz, 1H), 7.09 (t, J = 7.7 Hz, 2H), 6.97-6.90 (m, 1H), 4.42 (s, 2H), 3.92 (d, J = 12.7 Hz, 1H), 3.81 (d, J = 13.2 Hz, 2H), 3.50-3.33 (m, 2H), 3.19-3.09 (m, 3H), 2.49 (t, J = 10.1 Hz, 2H), 1.92 (t, J = 12.7 Hz, 1H), 1.76 (d, J = 11.2 Hz, 1H), 1.09 (t, J = 7.2 Hz, 3H), |
| | (3S,5R)-5-(1-cyclopropyl-3-(2-fluoro- | | 0.94 (s, 5H), 0.78-0.67 (m, |
| | 4-(trifluoromethoxy)benzyl)ureido)-N- | | 2H). |
| | ethyl-3-(hydroxymethyl)-3- | | |
| | methylpiperidine-1-carboxamide | | |
| Example 152 | 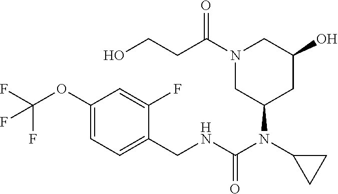 [SMILES: O=C(CCO)N1C[C@@H](O)C[C@@H](N(C(=O)NCc2ccc(OC(F)(F)F)cc2F)C2CC2)C1] | 464.2 | 1H NMR (400 MHz, MeOD) δ 7.43 (dd, J = 15.7, 8.2 Hz, 1H), 7.09 (t, J = 8.6 Hz, 2H), 6.97 (d, J = 21.2 Hz, 1H), 4.69-4.60 (m, 1H), 4.46 (s, 1H), 4.46- 4.39 (m, 2H), 3.99 (d, J = 4.7 Hz, 1H), 3.86 (t, J = 9.5 Hz, 1H), 3.81 (q, J = 5.9 Hz, 2H), 3.54 (d, J = 4.8 Hz, 1H), 3.25 (t, J = 12.1 Hz, 1H), |
| | 1-cyclopropyl-3-(2-fluoro-4- | | 3.04 (t, J = 11.9 Hz, |
| | (trifluoromethoxy)benzyl)-1-((3R,5S)- | | 1H), 2.79-2.73 (m, 1H), |
| | 5-hydroxy-1-(3- | | 2.69-2.50 (m, 3H), 2.30 |
| | hydroxypropanoyl)piperidin-3-yl)urea | | (t, J = 11.5 Hz, 1H), 2.23- |
| | | | 2.04 (m, 2H), 0.9 6 (dd, |
| | | | J = 11.8 , 6.4 Hz, 2H), |
| | | | 0.77 (d, J = 26.5 Hz, 2H). |
| Example 153 | 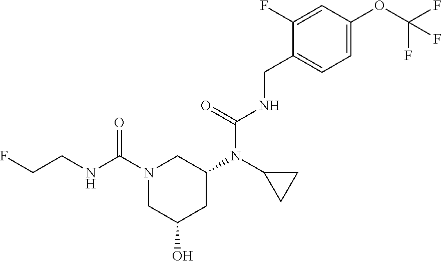 [SMILES: O=C(NCCF)N1C[C@@H](O)C[C@@H](N(C(=O)NCc2ccc(OC(F)(F)F)cc2F)C2CC2)C1] | 481.2 | 1H NMR (400 MHz, MeOD) δ 7.43 (t, J = 8.6 Hz, 1H), 7.09 (t, J = 7.3 Hz, 2H), 6.98 (t, J = 5.9 Hz, 1H), 4.49-4.38 (m, 3H), 4.34 (t, J = 5.2 Hz, 1H), 4.15 (dd, J = 12.7, 4.8 Hz, 1H), 3.83 (d, J = 12.8 Hz, 1H), 3.73-3.63 (m, 1H), 3.59-3.50 (m, 1H), 3.44 (dd, J = 5.3, 4.0 Hz, 1H), 3.38 (t, J = 5.2 Hz, 1H), 3.15-3.02 (m, 1H), 2.58-2.50 (m, 1H), 2.43 (dd, J = 12.7, 10.8 Hz, 1H), 2.15 (d, J = 11.6 Hz, 1H), |
| | (3R,5S)-3-(1-cyclopropyl-3-(2-fluoro- | | 2.04 (dd, J = 23.2, 11.7 Hz, |
| | 4-(trifluoromethoxy)benzyl)ureido)-N- | | 1H), 1.02-0.88 (m, 2H), |
| | (2-fluoroethyl)-5-hydroxypiperidine- | | 0.82-0.64 (m, 2H). |
| | 1-carboxamide | | |
| Example 154 | 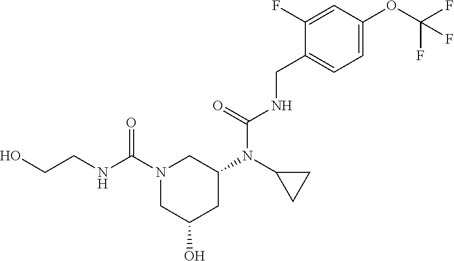 [SMILES: O=C(NCCO)N1C[C@@H](O)C[C@@H](N(C(=O)NCc2ccc(OC(F)(F)F)cc2F)C2CC2)C1] | 479.4 | 1H NMR (400 MHz, Methanol-d4) δ 7.49-7.40 (m, 1H), 7.14-7.02 (m, 2H), 4.52-4.36 (m, 2H), 4.21-4.11 (m, 1H), 3.89- 3.78 (m, 1H), 3.76-3.65 (m, 1H), 3.63-3.51 (m, 3H), 3.28-3.21 (m, 2H), 3.14-3.02 (m, 1H), 2.60- 2.49 (m, 1H), 2.48-2.36 (m, 1H), 2.20-2.12 (m, 1H), 2.09-1.96 (m, 1H), 1.02-0.90 (m, 2H), 0.83- 0.69 (m, 2H). |
| | (3R,5S)-3-(1-cyclopropyl-3-(2-fluoro- | | |
| | 4-(trifluoromethoxy)benzyl)ureido)-5- | | |
| | hydroxy-N-(2- | | |
| | hydroxyethyl)piperidine-1- | | |
| | carboxamide | | |
| Example 155 | 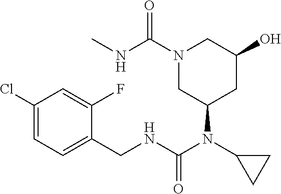 [SMILES: CNC(=O)N1C[C@@H](O)C[C@@H](N(C(=O)NCc2ccc(Cl)cc2F)C2CC2)C1] | 399.1 | 1H NMR (400 MHz, MeOD) δ 7.32 (t, J = 8.3 Hz, 1H), 7.16 (dt, J = 10.2, 2.1 Hz, 2H), 6.94 (t, J = 5.9 Hz, 1H), 4.40 (t, J = 5.0 Hz, 2H), 4.13 (dd, J = 12.7, 4.8 Hz, 1H), 3.78 (dd, J = 12.6, 4.1 Hz, 1H), 3.72- 3.62 (m, 1H), 3.58-3.46 (m, 1H), 3.12-3.00 (m, 1H), 2.69 (s, 3H), 2.51 (dt, |
| | (3R,5S)-3-(3-(4-chloro-2- | | J = 10.4, 3.4 Hz, 1H), 2.41 |
| | fluorobenzyl)-1-cyclopropylureido)-5- | | (dd, J = 12.6, 10.8 Hz, 1H), |
| | hydroxy-N-methylpiperidine-1- | | 2.14 (d, J = 11.5 Hz, 1H), |
| | carboxamide | | 2.02 (dd, J = 23.1, 11.8 Hz, |
| | | | 1H), 1.00-0.88 (m, 2H), |
| | | | 0.82-0.69 (m, 2H). |
| Example 156 | 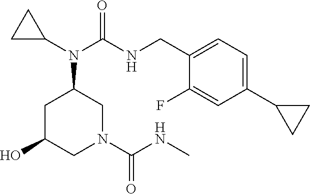 [SMILES: CNC(=O)N1C[C@@H](O)C[C@@H](N(C(=O)NCc2ccc(C3CC3)cc2F)C2CC2)C1] | 405.2 | 1H NMR (400 MHz, MeOD) δ 7.19 (t, J = 8.0 Hz, 1H), 6.89-6.83 (m, 1H), 6.82-6.77 (m, 1H), 6.75 (d, J = 1.6 Hz, 1H), 4.38 (t, J = 5.8 Hz, 2H), 4.19-4.10 (m, 1H), 3.82- 3.74 (m, 1H), 3.73-3.63 (m, 1H), 3.59-3.49 (m, 1H), 3.11-3.02 (m, 1H), 2.70 (s, 3H), 2.54-2.46 (m, 1H), 2.45-2.36 (m, |
| | (3R,5S)-3-(1-cyclopropyl-3-(4- | | 1H), 2.19-2.10 (m, 1H), |
| | cyclopropyl-2-fluorobenzyl)ureido)-5- | | 2.08-1.97 (m, 1H), 1.95- |
| | hydroxy-N-methylpiperidine-1- | | 1.84 (m, 1H), 1.01-0.88 |
| | carboxamide | | (m, 4H), 0.80-0.70 (m, |
| | | | 2H), 0.70-0.62 (m, 2H). |
| Example 157 | 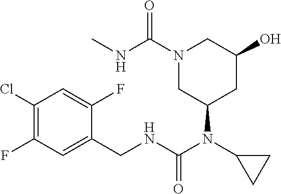 [SMILES: CNC(=O)N1C[C@@H](O)C[C@@H](N(C(=O)NCc2cc(F)c(Cl)cc2F)C2CC2)C1] | 417.2 | 1H NMR (400 MHz, MeOD) δ 7.29 (dd, J = 9.2, 6.0 Hz, 1H), 7.19 (dd, J = 9.5, 6.5 Hz, 1H), 4.38 (t, J = 6.8 Hz, 2H), 4.13 (dd, J = 12.7, 4.8 Hz, 1H), 3.79 (d, J = 12.7 Hz, 1H), 3.67 (tt, J = 12.0, 4.0 Hz, 1H), 3.58- 3.49 (m, 1H), 3.11-3.04 (m, 1H), 2.69 (s, 3H), 2.57- 2.51 (m, 1H), 2.41 (dd, J = 12.6, 10.8 Hz, 1H), 2.15 |
| | (3R,5S)-3-(3-(4-chloro-2,5- | | (d, J = 11.6 Hz, 1H), 2.05- |
| | difluorobenzyl)-1-cyclopropylureido)- | | 1.97 (m, 1H), 0.99-0.92 |
| | 5-hydroxy-N-methylpiperidine-1- | | (m, 2H), 0.80-0.73 (m, |
| | carboxamide | | 2H). |
| Example 158 | 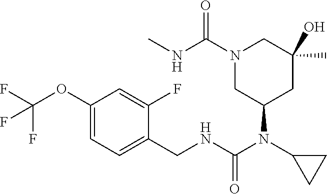 [SMILES: CNC(=O)N1C[C@H](N(C(=O)NCc2ccc(OC(F)(F)F)cc2F)C2CC2)C[C@](C)(O)C1] | 463.2 | 1HNMR (400 MHz, MeOD) δ 7.43 (t, J = 8.6 Hz, 1H), 7.09 (t, J = 7.3 Hz, 2H), 4.51- 4.36 (m, 2H), 3.92-3.73 (m, 3H), 3.0 9 (t, J = 12.0 Hz, 1H), 2.70 (s, 3H), 2.58 (d, J = 12.9 Hz, 1H), 2.54-2.47 (m, 1H), 2.21 (t, J = 12.4 Hz, 1H), 1.89-1.80 (m, 1H), 1.21 (s, 3H), 0.96 (dd, J = 6.6, 1.9 Hz, 2H), 0.81- 0.71 (m, 2H). |
| | (3S,5R)-5-(1-cyclopropyl-3-(2-fluoro- | | |
| | 4-(trifluoromethoxy)benzyl)ureido)-3- | | |
| | hydroxy-N,3-dimethylpiperidine-1- | | |
| | carboxamide | | |
| Example 159, | 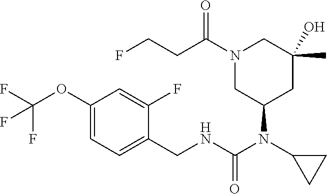 [SMILES: C[C@@]1(O)C[C@@H](N(C(=O)NCc2ccc(OC(F)(F)F)cc2F)C2CC2)CN(C(=O)CCF)C1] | 480.4 | 1HNMR (400 MHz, MeOD) δ 7.44 (q, J = 8.6 Hz, 1H), 7.09 (t, J = 7.3 Hz, 2H), 6.98 (dt, J = 18.4, 5.7 Hz, 1H), 4.76 (dd, J = 10.4, 4.7 Hz, 1H), 4.67-4.60 (m, 1H), 4.54-4.34 (m, 3H), 3.86 (dt, J = 8.4, 7.4 Hz, 1H), 3.72-3.61 (m, 1H), 3.28 (s, 1H), 3.08 (t, J = 12.0 Hz, 1H), 3.00-2.68 (m, 2H), 2.59-2.44 (m, |
| | 1-cyclopropyl-3-(2-fluoro-4- | | 2H), 2.30 (dd, J = 21.7, |
| | (trifluoromethoxy)benzyl)-1-((3R,5R)- | | 12.4 Hz, 1H), 1.87 (dd, J = |
| | 1-(3-fluoropropanoyl)-5-hydroxy-5- | | 21.1, 12.5 Hz, 1H), 1.22 (d, |
| | methylpiperidin-3-yl)urea | | J = 26.2 Hz, 3H), 1.01- |
| | | | 0.89 (m, 2H), 0.83-0.67 |
| | | | (m, 2H). |
| Example 160 | 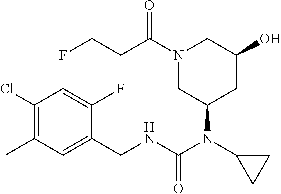 [SMILES: Cc1cc(CNC(=O)N(C2CC2)[C@@H]2C[C@H](O)CN(C(=O)CCF)C2)c(F)cc1Cl] | 430.2 | 1H NMR (400 MHz, MeOD) δ 7.24 (t, J = 7.9 Hz, 1H), 7.13 (dd, J = 9.8, 3.4 Hz, 1H), 4.75 (dd, J = 11.0, 5.6 Hz, 1H), 4.63 (dd, J = 10.9, 5.6 Hz, 1.5H), 4.44 (dd, J = 11.7, 4.7 Hz, 0.5H), 4.36 (t, J = 11.9 Hz, 2H), 4.00-3.81 (m, 1H), 3.80-3.73 (m, 0.5H), 3.55 (ddd, J = 15.6, 13.3, 7.4 Hz, |
| | 3-(4-chloro-2-fluoro-5-methylbenzyl)- | | 1.5H), 3.29-3.20 (m, |
| | 1-cyclopropyl-1-((3R,5S)-1-(3- | | 1H), 3.09-2.74 (m, 3H), |
| | fluoropropanoyl)-5-hydroxypiperidin- | | 2.53 (dtd, J = 14.1, 6.7, |
| | 3-yl)urea | | 3.5 Hz, 1H), 2.32 (s, 3H), |
| | | | 2.14 (ddd, J = 23.4, 21.3, |
| | | | 11.6 Hz, 2H), 0.99-0.90 |
| | | | (m, 2H), 0.83-0.67 (m, 2H). |
| Example 161 (cis) | 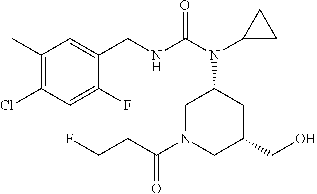 [SMILES: Cc1cc(CNC(=O)N(C2CC2)[C@@H]2C[C@H](CO)CN(C(=O)CCF)C2)c(F)cc1Cl] | 444.2 | 1H NMR (400 MHz, CD3OD) δ 7.25 (t, J = 8.5 Hz, 1H), 7.18-7.10 (m, 1H), 4.78-4.74 (m, 1H), 4.68-4.62 (m, 2H), 4.54- 4.48 (m, 1H), 4.43-4.33 (m, 2H), 4.05 (d, J = 13.3 Hz, 1H), 3.90 (d, J = 12.8 Hz, 1H), 3.82- 3.75 (m, 1H), 3.61-3.38 (m, 3H), 3.14-3.08 (m, 1H), 2.91-2.72 (m, 2H), |
| | 3-(4-chloro-2-fluoro-5-methylbenzyl)- | | 2.60-2.49 (m, 1H), 2.33 |
| | 1-cyclopropyl-1-((3R,5S)-1-(3- | | (s, 3H), 2.27 (d, J = |
| | fluoropropanoyl)-5- | | 12.3 Hz, 1H), 2.02- |
| | (hydroxymethyl)piperidin-3-yl)urea | | 1.85 (m, 2H), 1.81-1.67 |
| | | | (m, 1H), 1.00-0.90 (m, |
| | | | 2H), 0.83-0.67 (m, 2H). |
| Example 162 (cis) | 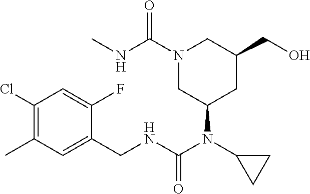 [SMILES: CNC(=O)N1C[C@@H](CO)C[C@@H](N(C(=O)NCc2cc(C)c(Cl)cc2F)C2CC2)C1] | 427.1 | 1H NMR (400 MHz, Methanol-d4) δ 7.29-7.20 (m, 1H), 7.17-7.08 (m, 1H), 4.43-4.31 (m, 2H), 4.14-4.04 (m, 1H), 3.94- 3.83 (m, 1H), 3.76-3.61 (m, 1H), 3.51-3.38 (m, 2H), 3.18-3.07 (m, 1H), 2.70 (s, 3H), 2.61-2.48 (m, 1H), 2.45-2.35 (m, 1H), 2.32 (s, 3H), 1.96- 1.79 (m, 2H), 1.79-1.64 |
| | (3R,5S)-3-(3-(4-chloro-2-fluoro-5- | | (m, 1H), 0.98-0.88 (m, |
| | methylbenzyl)-1-cyclopropylureido)- | | 2H), 0.79-0.67 (m, 2H). |
| | 5-(hydroxymethyl)-N- | | |
| | methylpiperidine-1-carboxamide | | |
| Example 163 | 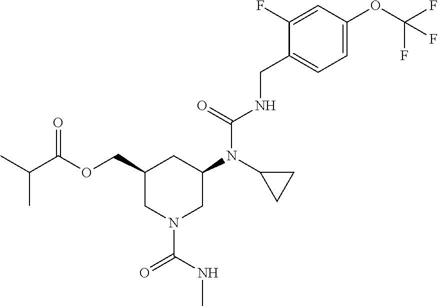 [SMILES: CNC(=O)N1C[C@@H](COC(=O)C(C)C)C[C@@H](N(C(=O)NCc2ccc(OC(F)(F)F)cc2F)C2CC2)C1] | 533.3 | 1H NMR (400 MHz, MeOD) δ 7.43 (t, J = 8.6 Hz, 1H), 7.09 (t, J = 7.3 Hz, 2H), 6.97 (t, J = 5.8 Hz, 1H), 4.44 (s, 2H), 4.11 (d, J = 12.7 Hz, 1H), 4.05-3.93 (m, 2H), 3.88- 3.80 (m, 1H), 3.73-3.62 (m, 1H), 3.20-3.10 (m, 1H), 2.70 (s, 3H), 2.63-2.51 (m, 2H), 2.48-2.36 (m, 1H), 1.92 (d, J = 7.7 Hz, 3H), 1.17 (d, J = 7.0 Hz, 6H), 0.98-0.91 (m, 2H), 0.83-0.72 (m, 2H). |
| | ((3S,5R)-5-(1-cyclopropyl-3-(2-fluoro- | | |
| | 4-(trifluoromethoxy)benzyl)ureido)-1- | | |
| | (methylcarbamoyl)piperidin-3- | | |
| | yl)methyl isobutyrate | | |
| Example 164 | 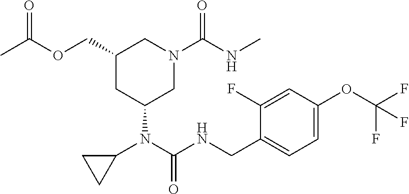 [SMILES: CNC(=O)N1C[C@@H](COC(C)=O)C[C@@H](N(C(=O)NCc2ccc(OC(F)(F)F)cc2F)C2CC2)C1] | 505.3 | 1H NMR (400 MHz, MeOD) δ 7.43 (t, J = 8.6 Hz, 1H), 7.09 (t, J = 7.9 Hz, 2H), 4.41 (d, J = 16.5 Hz, 2H), 4.08 (d, J = 13.1 Hz, 1H), 4.02-3.90 (m, 2H), 3.86 (d, J = 12.3 Hz, 1H), 3.71-3.61 (m, 1H), 3.15 (t, J = 12.0 Hz, 1H), 2.70 (s, 3H), 2.60-2.51 (m, 1H), 2.48-2.38 (m, 1H), 2.05 (s, 3H), 1.90 (d, J = 5.2 Hz, 3H), 0.99-0.92 |
| | ((3S,5R)-5-(1-cyclopropyl-3-(2-fluoro- | | (m, 2H), 0.79-0.71 (m, |
| | 4-(trifluoromethoxy)benzyl)ureido)-1- | | 2H). |
| | (methylcarbamoyl)piperidin-3- | | |
| | yl)methyl acetate | | |
| Example 165 | 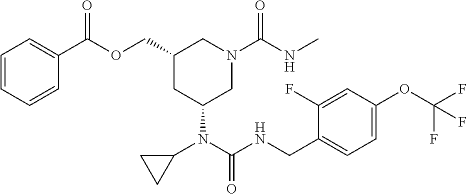 [SMILES: CNC(=O)N1C[C@@H](COC(=O)c2ccccc2)C[C@@H](N(C(=O)NCc2ccc(OC(F)(F)F)cc2F)C2CC2)C1] | 567.2 | 1H NMR (400 MHz, MeOD) δ 8.10-8.01 (m, 2H), 7.61 (d, J = 7.4 Hz, 1H), 7.52-7.41 (m, 3H), 7.08 (d, J = 9.7 Hz, 2H), 4.44 (s, 2H), 4.31-4.17 (m, 3H), 3.88 (d, J = 9.0 Hz, 1H), 3.72 (d, J = 4.5 Hz, 1H), 3.20 (s, 1H), 2.71 (s, 3H), 2.61-2.51 (m, 2H), 2.13-1.97 (m, 3H), 0.96 (d, J = 4.2 Hz, 2H), 0.82-0.72 (m, 2H). |
| | ((3S,5R)-5-(1-cyclopropyl-3-(2-fluoro- | | |
| | 4-(trifluoromethoxy)benzyl)ureido)-1- | | |
| | (methylcarbamoyl)piperidin-3- | | |
| | yl)methyl benzoate | | |
| Example 166 | 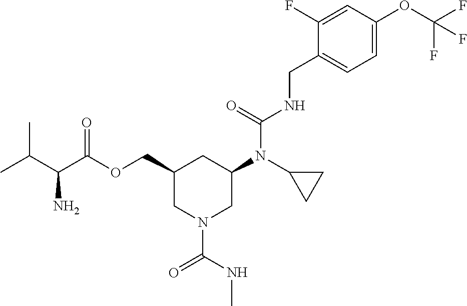 [SMILES: CNC(=O)N1C[C@@H](COC(=O)[C@@H](N)C(C)C)C[C@@H](N(C(=O)NCc2ccc(OC(F)(F)F)cc2F)C2CC2)C1] | 562.3 | 1H NMR (400 MHz, MeOD) δ 8.46 (s, 1H), 7.43 (t, J = 8.6 Hz, 1H), 7.14-7.04 (m, 2H), 6.99 (t, J = 5.8 Hz, 1H), 4.44 (d, J = 5.3 Hz, 2H), 4.18- 4.06 (m, 3H), 3.88-3.76 (m, 2H), 3.69-3.60 (m, 1H), 3.24-3.16 (m, 1H), 2.71 (s, 3H), 2.59-2.52 (m, 1H), 2.48-2.39 (m, 1H), 2.28-2.18 (m, 1H), 1.99-1.88 (m, 3H), 1.07- 1.01 (m, 6H), 0.98-0.93 (m, 2H), 0.81-0.72 (m, 2H). |
| | ((3S,5R)-5-(1-cyclopropyl-3-(2-fluoro- | | |
| | 4-(trifluoromethoxy)benzyl)ureido)-1- | | |
| | (methylcarbamoyl)piperidin-3- | | |
| | yl)methyl L-valinate | | |
| Example 167 | 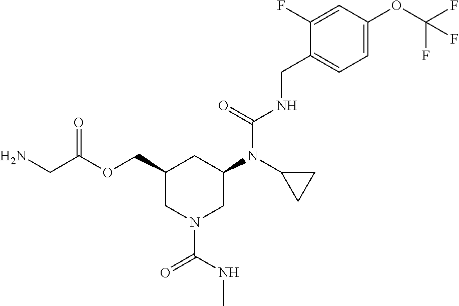 [SMILES: CNC(=O)N1C[C@@H](COC(=O)CN)C[C@@H](N(C(=O)NCc2ccc(OC(F)(F)F)cc2F)C2CC2)C1] | 520.2 | 1H NMR (400 MHz, MeOD) δ 8.43 (s, 1H), 7.43 (dd, J = 10.7, 6.5 Hz, 1H), 7.09 (dd, J = 7.3, 4.7 Hz, 2H), 6.99 (t, J = 5.9 Hz, 1H), 4.44 (d, J = 5.6 Hz, 2H), 4.24-4.04 (m, 3H), 3.89-3.73 (m, 3H), 3.70-3.60 (m, 1H), 3.25- 3.15 (m, 1H), 2.71 (s, 3H), 2.59-2.51 (m, 1H), 2.49- 2.35 (m, 1H), 1.98-1.84 (m, 3H), 0.95 (dd, J = 6.2, 3.3 Hz, 2H), 0.83-0.67 (m, 2H). |
| | ((3S,5R)-5-(1-cyclopropyl-3-(2-fluoro- | | |
| | 4-(trifluoromethoxy)benzyl)ureido)-1- | | |
| | (methylcarbamoyl)piperidin-3- | | |
| | yl)methyl glycinate | | |
| Example 169 | 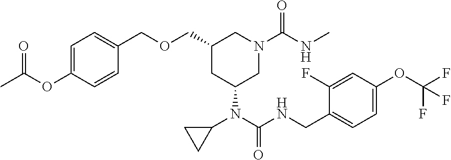 [SMILES: CNC(=O)N1C[C@@H](COCc2ccc(OC(C)=O)cc2)C[C@@H](N(C(=O)NCc2ccc(OC(F)(F)F)cc2F)C2CC2)C1] | 611.4 | 1H NMR (400 MHz, MeOD) δ 7.42 (t, J = 8.6 Hz, 1H), 7.37 (d, J = 8.2 Hz, 2H), 7.07 (d, J = 8.1 Hz, 4H), 6.94 (t, J = 5.7 Hz, 1H), 4.48 (d, J = 12.7 Hz, 2H), 4.44 (t, J = 7.1 Hz, 2H), 4.09 (d, J = 13.3 Hz, 1H), 3.88 (d, J = 12.8 Hz, |
| | 4-((((3S,5R)-5-(1-cyclopropyl-3-(2- | | 1H), 3.65 (s, 1H), 3.44- |
| | fluoro-4- | | 3.33 (m, 2H), 3.11 (t, J = |
| | (trifluoromethoxy)benzyl)ureido)-1- | | 12.0 Hz, 1H), 2.69 (s, 3H), |
| | (methylcarbamoyl)piperidin-3- | | 2.52 (s, 1H), 2.48-2.37 |
| | yl)methoxy)methyl)phenyl acetate | | (m, 1H), 2.26 (s, 3H), 1.90 |
| | | | (t, J = 8.5 Hz, 3H), 0.93 (d, |
| | | | J = 4.2 Hz, 2H), 0.75 (s, |
| | | | 2H). |
| Example 170 | 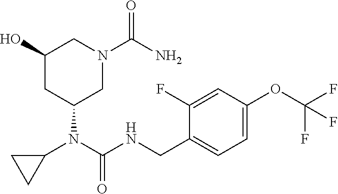 [SMILES: NC(=O)N1C[C@H](O)C[C@@H](N(C(=O)NCc2ccc(OC(F)(F)F)cc2F)C2CC2)C1] | 435.2 | 1H NMR (400 MHz, MeOD) δ 7.43 (t, J = 8.6 Hz, 1H), 7.08 (t, J = 8.1 Hz, 2H), 6.93 (t, J = 5.8 Hz, 1H), 4.43 (d, J = 5.7 Hz, 2H), 4.07-3.89 (m, 4H), 3.29-3.21 (m, 1H), 2.88 (d, J = 13.2 Hz, 1H), 2.60-2.50 (m, 1H), 2.38 (td, J = 12.9, 3.0 Hz, 1H), 1.91 (d, J = 12.9 Hz, 1H), 0.93 (t, J = 5.9 Hz, 2H), |
| | (3R,5R)-3-(1-cyclopropyl-3-(2-fluoro- | | 0.82-0.68 (m, 2H). |
| | 4-(trifluoromethoxy)benzyl)ureido)-5- | | |
| | hydroxypiperidine-1-carboxamide | | |
| Example 171 (cis) | 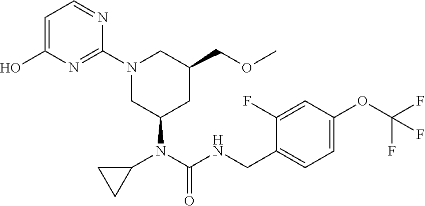 [SMILES: COC[C@H]1C[C@@H](N(C(=O)NCc2ccc(OC(F)(F)F)cc2F)C2CC2)CN(c2nccc(O)n2)C1] | 514.2 | 1H NMR (400 MHz, MeOD) δ 7.59 (s, 1H), 7.44 (t, J = 8.6 Hz, 1H), 7.09 (t, J = 7.7 Hz, 2H), 7.01 (t, J = 5.8 Hz, 1H), 5.74 (d, J = 6.7 Hz, 1H), 4.41 (d, J = 21.4 Hz, 3H), 4.25 (d, J = 12.2 Hz, 1H), 3.74 (t, J = 11.4 Hz, 1H), 3.35 (q,J = 4.5 Hz, 5H), 3.27 (s, 1H), 2.63 (dt, J = 16.6, 8.4 Hz, 1H), 2.59- 2.52 (m, 1H), 1.99 (d, J = |
| | 1-cyclopropyl-3-(2-fluoro-4- | | 14.0 Hz, 1H), 1.95 (d, J = |
| | (trifluoromethoxy)benzyl)-1-((3R,5S)- | | 5.3 Hz, 2H), 0.95 (dd, J = |
| | 1-(4-hydroxypyrimidin-2-yl)-5- | | 6.1, 3.4 Hz, 2H), 0.79 (d, |
| | (methoxymethyl)piperidin-3-yl)urea | | J = 3.7 Hz, 2H). |
| Example 172 (cis) | 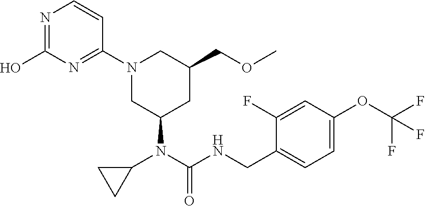 [SMILES: COC[C@H]1C[C@@H](N(C(=O)NCc2ccc(OC(F)(F)F)cc2F)C2CC2)CN(c2ccnc(O)n2)C1] | 514.3 | 1H NMR (400 MHz, MeOD) δ 7.51-7.36 (m, 2H), 7.09 (t, J = 8.6 Hz, 2H), 6.18 (d, J = 6.6 Hz, 1H), 4.25 (d, J = 149.7 Hz, 4H), 3.64 (s, 1H), 3.33 (s, 5H), 3.29-3.27 (m, 1H), 2.57 (s, 2H), 2.06-1.98 (m, 1H), 1.89 (s, 2H), 0.96 (s, 2H), 0.78 (s, 2H). |
| | 1-cyclopropyl-3-(2-fluoro-4- | | |
| | (trifluoromethoxy)benzyl)-1-((3R,5S)- | | |
| | 1-(2-hydroxypyrimidin-4-yl)-5- | | |
| | (methoxymethyl)piperidin-3-yl)urea | | |
| Example 173 (cis) | 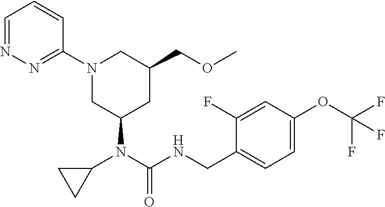 [SMILES: COC[C@H]1C[C@@H](N(C(=O)NCc2ccc(OC(F)(F)F)cc2F)C2CC2)CN(c2cccnn2)C1] | 498.3 | 1H NMR (400 MHz, MeOD) δ 8.43 (d, J = 3.9 Hz, 1H), 7.45 (t, J = 8.6 Hz, 1H), 7.37 (dd, J = 9.4, 4.4 Hz, 1H), 7.28 (d, J = 8.5 Hz, 1H), 7.09 (t, J = 8.8 Hz, 2H), 6.97 (d, J = 5.9 Hz, 1H), 4.46 (t, J = 7.9 Hz, 3H), 4.36 (d, J = 12.6 Hz, 1H), 3.85-3.74 (m, 1H), 3.36- 3.33 (m, 5H), 3.27 (s, 1H), 2.66 (dd, J = 13.2, 10.9 Hz, |
| | 1-cyclopropyl-3-(2-fluoro-4- | | 1H), 2.57 (td, J = 6.6, |
| | (trifluoromethoxy)benzyl)-1-((3R,5S)- | | 3.5 Hz, 1H), 2.06-1.91 |
| | 5-(methoxymethyl)-1-(pyridazin-3- | | (m, 3H), 0.96 (t, J = 6.6 Hz, |
| | yl)piperidin-3-yl)urea | | 2H), 0.84-0.76 (m, 2H). |
| Example 174 | 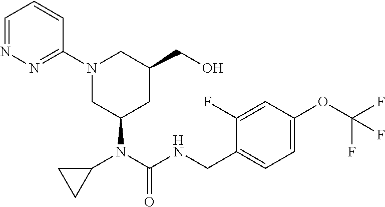 [SMILES: O=C(NCc1ccc(OC(F)(F)F)cc1F)N(C1CC1)[C@@H]1C[C@H](CO)CN(c2cccnn2)C1] | 484.3 | 1H NMR (400 MHz, CD3OD) δ 8.47-8.41 (m, 1H), 7.45 (t, J = 8.5 Hz, 1H), 7.40-7.34 (m, 1H), 7.31-7.27 (m, 1H), 7.10 (t, J = 8.8 Hz, 2H), 4.54- 4.48 (m, 1H), 4.46 (s, 2H), 4.42-4.35 (m, 1H), 3.86- 3.76 (m, 1H), 3.56-3.47 (m, 2H), 3.33 (s, 1H), 2.70- 2.61 (m, 1H), 2.61-2.55 (m, 1H), 2.04-1.94 (m, 2H), 1.90-1.80 (m, 1H), |
| | 1-cyclopropyl-3-(2-fluoro-4- | | 1.00-0.93 (m, 2H), 0.85- |
| | (trifluoromethoxy)benzyl)-1-((3R,5S)- | | 0.77 (m, 2H). |
| | 5-(hydroxymethyl)-1-(pyridazin-3- | | |
| | yl)piperidin-3-yl)urea | | |
| Example 175 (cis) | 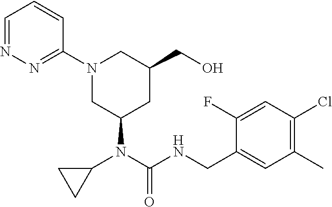 [SMILES: Cc1cc(CNC(=O)N(C2CC2)[C@@H]2C[C@H](CO)CN(c3cccnn3)C2)c(F)cc1Cl] | 448.2 | 1H NMR (400 MHz, CD3OD) δ 8.44 (d, J = 4.1 Hz, 1H), 8.16 (s, 1H), 7.42-7.34 (m, 1H), 7.33- 7.21 (m, 2H), 7.13 (d, J = 9.8 Hz, 1H), 6.90 (t, J = 5.8 Hz, 1H), 4.50 (d, J = 10.6 Hz, 1H), 4.38 (t, J = 6.8 Hz, 3H), 3.86-3.76 (m, 1H), 3.57-3.46 (m, 2H), 3.33 (s, 1H), 2.69-2.60 (m, 1H), 2.60-2.52 (m, 1H), 2.32 (s, 3H), 2.05- |
| | 3-(4-chloro-2-fluoro-5-methylbenzyl)- | | 1.93 (m, 2H), 1.88-1.75 |
| | 1-cyclopropyl-1-((3R,5S)-5- | | (m, 1H), 0.95 (t, J = |
| | (hydroxymethyl)-1-(pyridazin-3- | | 6.8 Hz, 2H), 0.83-0.73 |
| | yl)piperidin-3-yl)urea | | (m, 2H). |
| Example 176 | 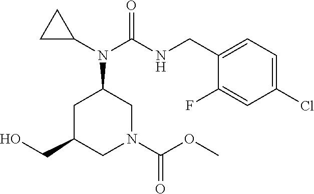 [SMILES: COC(=O)N1C[C@@H](CO)C[C@@H](N(C(=O)NCc2ccc(Cl)cc2F)C2CC2)C1] | 414.1 | 1H NMR (400 MHz, CD3OD) δ 7.32 (t, J = 8.3 Hz, 1H), 7.21-7.12 (m, 2H), 4.40 (s, 2H), 4.21 (d, J = 11.8 Hz, 1H), 4.05 (d, J = 9.1 Hz, 1H), 3.68 (s, 4H), 3.50-3.37 (m, 2H), 3.17 (t, J = 11.8 Hz, 1H), 2.48 (d, J = 40.8 Hz, 2H), 1.85 (t, J = 9.8 Hz, 2H), 1.74 (d, J = 4.7 Hz, 1H), 0.99- 0.89 (m, 2H), 0.74 (s, 2H). |
| | methyl (3R,5S)-3-(3-(4-chloro-2- | | |
| | fluorobenzyl)-1-cyclopropylureido)-5- | | |
| | (hydroxymethyl)piperidine-1- | | |
| | carboxylate | | |
| Example 177 (cis) | 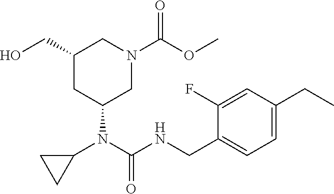 [SMILES: CCc1ccc(CNC(=O)N(C2CC2)[C@@H]2C[C@H](CO)CN(C(=O)OC)C2)c(F)c1] | 408.2 | 1H NMR (400 MHz, CD3OD) δ 7.22 (t, J = 7.8 Hz, 1H), 6.98 (d, J = 7.8 Hz, 1H), 6.91 (d, J = 11.5 Hz, 1H), 6.75 (s, 1H), 4.40 (d, J = 4.2 Hz, 2H), 4.20 (d, J = 12.1 Hz, 1H), 4.05 (d, J = 8.3 Hz, 1H), 3.68 (s, 4H), 3.51-3.37 (m, 0 H), 3.18 (t, J = 12.2 Hz, 1H), 2.63 (q, J = 7.6 Hz, 2H), 2.47 (d, J = 38.1 Hz, 2H), 1.92- |
| | methyl (3R,5S)-3-(1-cyclopropyl-3-(4- | | 1.79 (m, 2H), 1.73 (s, 1H), 1.22 |
| | ethyl-2-fluorobenzyl)ureido)-5- | | (t, J = 7.6 Hz, 3H), 0.92 (s, 2H), |
| | (hydroxymethyl)piperidine-1- | | 0.73 (s, 2H). |
| | carboxylate | | |
| Example 178 (cis) | 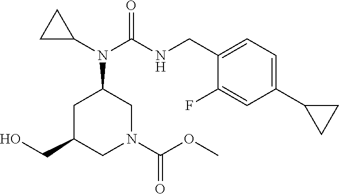 [SMILES: COC(=O)N1C[C@@H](CO)C[C@@H](N(C(=O)NCc2ccc(C3CC3)cc2F)C2CC2)C1] | 420.2 | 1H NMR (400 MHz, CD3OD) δ 7.18 (t, J = 8.0 Hz, 1H), 6.86 (d, J = 7.9 Hz, 1H), 6.76 (d, J = 12.8 Hz, 1H), 4.37 (s, 2H), 4.20 (d, J = 14.4 Hz, 1H), 4.04 (d, J = 11.7 Hz, 1H), 3.68 (s, 4H), 3.50-3.37 (m, 2H), 3.17 (t, J = 12.0 Hz, 1H), 2.47 (d, J = 36.8 Hz, 2H), 1.94-1.79 (m, 3H), 1.73 (s, 1H), 1.00-0.89 |
| | methyl (3R,5S)-3-(1-cyclopropyl-3-(4- | | (m, 4H), 0.75-0.61 (m, |
| | cyclopropyl-2-fluorobenzyl)ureido)-5- | | 4H). |
| | (hydroxymethyl)piperidine-1- | | |
| | carboxylate | | |
| Example 179 (cis) | 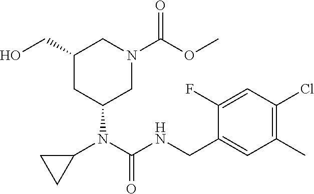 [SMILES: COC(=O)N1C[C@@H](CO)C[C@@H](N(C(=O)NCc2cc(C)c(Cl)cc2F)C2CC2)C1] | 428.1 | 1H NMR (400 MHz, MeOD) δ 7.23 (d, J = 8.1 Hz, 1H), 7.13 (d, J = 9.8 Hz, 1H), 4.36 (s, 2H), 4.19 (s, 1H), 4.04 (s, 1H), 3.67 (d, J = 3.3 Hz, 4H), 3.50-3.39 (m, 2H), 3.21- 3.12 (m, 1H), 2.48 (d, J = 37.1 Hz, 2H), 2.32 (s, 3H), 1.90-1.66 (m, 3H), 0.93 (d, J = 6.3 Hz, 2H), 0.72 (s, 2H). |
| | methyl (3R,5S)-3-(3-(4-chloro-2- | | |
| | fluoro-5-methylbenzyl)-1- | | |
| | cyclopropylureido)-5- | | |
| | (hydroxymethyl)piperidine-1- | | |
| | carboxylate | | |
| Example 180 (cis) | 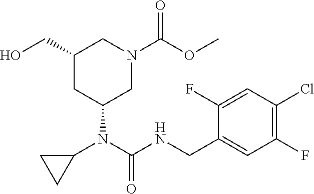 [SMILES: COC(=O)N1C[C@@H](CO)C[C@@H](N(C(=O)NCc2cc(F)c(Cl)cc2F)C2CC2)C1] | 432.3 | 1H NMR (400 MHz, MeOD) δ 7.29 (dd, J = 9.1, 6.1 Hz, 1H), 7.19 (dd, J = 9.3, 6.5 Hz, 1H), 6.97 (s, 1H), 4.38 (s, 2H), 4.22 (s, 1H), 4.04 (s, 1H), 3.68 (s, 4H), 3.46 (dt, J = 24.0, 11.9 Hz, 2H), 3.17 (t, J = 12.1 Hz, 1H), 2.49 (d, J = 45.6 Hz, 2H), 1.85 (t, J = 9.7 Hz, 2H), 1.73 (s, 1H), 0.95 (d, J = 5.2 Hz, 2H), 0.75 (s, 2H). |
| | methyl (3R,5S)-3-(3-(4-chloro-2,5- | | |
| | difluorobenzyl)-1-cyclopropylureido)- | | |
| | 5-(hydroxymethyl)piperidine-1- | | |
| | carboxylate | | |
| Example 181 | 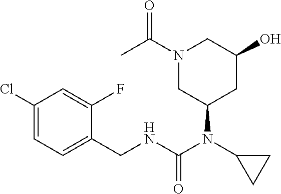 [SMILES: CC(=O)N1C[C@@H](O)C[C@@H](N(C(=O)NCc2ccc(Cl)cc2F)C2CC2)C1] | 384.1 | 1H NMR (400 MHz, MeOD) δ 7.32 (q, J = 7.8 Hz, 1H), 7.17 (d, J = 8.4 Hz, 2H), 4.61 (d, J = 8.5 Hz, 1H), 4.48-4.34 (m, 3H), 3.90 (d, J = 13.2 Hz, 1H), 3.78 (d, J = 11.2 Hz, 1H), 3.65-3.46 (m, 2H), 3.23 (t, J = 12.5 Hz, 1H), 3.01 (t, J = 11.8 Hz, 1H), 2.83-2.73 (m, 1H), 2.59- 2.47 (m, 1H), 2.28 (t, J = |
| | 1-((3R,5S)-1-acetyl-5- | | 11.5 Hz, 1H), 2.20 (d, J = |
| | hydroxypiperidin-3-yl)-3-(4-chloro-2- | | 11.9 Hz, 1H), 2.12 (dd, J = |
| | fluorobenzyl)-1-cyclopropylurea | | 12.9, 5.9 Hz, 4H), 2.07 (d, |
| | | | J = 11.6 Hz, 1H), 1.00-0.89 |
| | | | (m, 2H), 0.84-0.66 (m, 2H). |
| Example 182 | 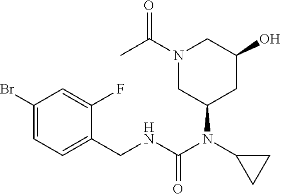 [SMILES: CC(=O)N1C[C@@H](O)C[C@@H](N(C(=O)NCc2ccc(Br)cc2F)C2CC2)C1] | 428.0 | 1H NMR (400 MHz, MeOD) δ 7.28 (dt, J = 22.9, 7.6 Hz, 3H), 4.61 (d, J = 8.5 Hz, 1H), 4.46-4.33 (m, 3H), 3.90 (d, J = 13.0 Hz, 1H), 3.78 (d, J = 11.6 Hz, 1H), 3.53 (ddd, J = 15.4, 12.9, 8.1 Hz, 2H), 3.23 (t, J = 12.6 Hz, 1H), 3.01 (t, J = 11.8 Hz, 1H), 2.84-2.73 (m, 1H), 2.61- 2.46 (m, 1H), 2.28 (t, |
| | 1-((3R,5S)-1-acetyl-5- | | J = 11.5 Hz, 1H), 2.20 (d, |
| | hydroxypiperidin-3-yl)-3-(4-bromo-2- | | J = 11.5 Hz, 1H), 2.12 (dd, |
| | fluorobenzyl)-1-cyclopropylurea | | J = 13.0, 6.2 Hz, 4H), 2.07 |
| | | | (d, J = 11.9 Hz, 1H), 0.96 |
| | | | (d, J = 16.1 Hz, 2H), 0.76 |
| | | | (dt, J = 24.6, 10.2 Hz, 2H). |
| Example 183 (trans) | 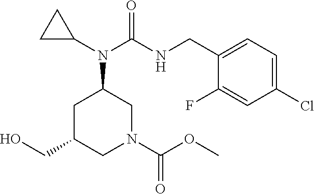 [SMILES: COC(=O)N1C[C@H](CO)C[C@@H](N(C(=O)NCc2ccc(Cl)cc2F)C2CC2)C1] | 414.2 | 1H NMR (400 MHz, MeOD) δ 7.32 (t, J = 8.3 Hz, 1H), 7.19-7.13 (m, 2H), 6.89 (s, 1H), 4.40 (d, J = 5.3 Hz, 2H), 4.02 (d, J = 13.0 Hz, 2H), 3.79 (s, 1H), 3.67 (s, 3H), 3.57-3.46 (m, 2H), 3.23 (t, J = 12.0 Hz, 1H), 2.94 (s, 1H), 2.50 (s, 1H), 2.32- 2.20 (m, 1H), 2.04-1.86 (m, 2H), 0.93 (d, J = |
| | methyl (3R,5R)-3-(3-(4-chloro-2- | | 6.5 Hz, 2H), 0.74 (s, 2H). |
| | fluorobenzyl)-1-cyclopropylureido)-5- | | |
| | (hydroxymethyl)piperidine-1- | | |
| | carboxylate | | |
| Example 184 (trans) | 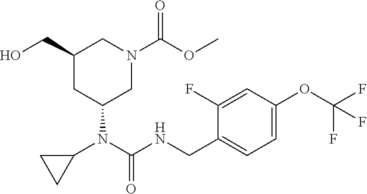 [SMILES: COC(=O)N1C[C@H](CO)C[C@@H](N(C(=O)NCc2ccc(OC(F)(F)F)cc2F)C2CC2)C1] | 464.2 | 1H NMR (400 MHz, MeOD) δ 7.43 (t, J = 8.6 Hz, 1H), 7.09 (t, J = 8.5 Hz, 2H), 4.43 (s, 2H), 4.03 (d, J = 12.9 Hz, 2H), 3.83 (d, J = 17.1 Hz, 1H), 3.67 (s, 3H), 3.60-3.45 (m, 2H), 3.24 (t, J = 11.8 Hz, 1H), 2.95 (s, 1H), 2.52 (s, 1H), 2.33-2.20 (m, 1H), 1.96 (d, J = 35.1 Hz, 2H), 0.94 (d, J = 6.5 Hz, 2H), |
| | methyl (3R,5R)-3-(1-cyclopropyl-3-(2- | | 0.75 (s, 2H). |
| | fluoro-4- | | |
| | (trifluoromethoxy)benzyl)ureido)-5- | | |
| | (hydroxymethyl)piperidine-1- | | |
| | carboxylate | | |
| Example 185 | 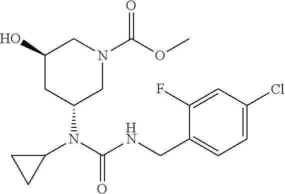 [SMILES: COC(=O)N1C[C@H](O)C[C@@H](N(C(=O)NCc2ccc(Cl)cc2F)C2CC2)C1] | 400.1 | 1H NMR (400 MHz, MeOD) δ 7.32 (t, J = 8.3 Hz, 1H), 7.19-7.13 (m, 2H), 6.91 (t, J = 5.7 Hz, 1H), 4.40 (s, 2H), 4.17 (s, 1H), 3.97 (s, 1H), 3.69 (d, J = 12.7 Hz, 4H), 3.55 (ddd, J = 15.4, 10.4, 4.8 Hz, 1H), 3.11 (t, J = 11.6 Hz, 1H), 2.49 (d, J = 30.3 Hz, 2H), 2.16-1.98 (m, 2H), 0.94 (dd, J = 6.6, 2.4 Hz, 2H), |
| | methyl (3R,5S)-3-(3-(4-chloro-2- | | 0.74 (s, 2H). |
| | fluorobenzyl)-1-cyclopropylureido)-5- | | |
| | hydroxypiperidine-1-carboxylate | | |
| Example 186 (cis) | 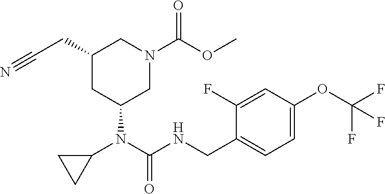 [SMILES: COC(=O)N1C[C@@H](CC#N)C[C@@H](N(C(=O)NCc2ccc(OC(F)(F)F)cc2F)C2CC2)C1] | 473.1 | 1H NMR (400 MHz, Methanol-d4) δ 7.47-7.38 (m, 1H), 7.14-7.03 (m, 2H), 4.43 (s, 2H), 4.26- 4.11 (m, 1H), 4.11-4.00 (m, 1H), 3.78-3.58 (m, 4H), 3.35-3.31 (m, 1H), 3.26-3.15 (m, 1H), 2.59- 2.53 (m, 1H), 2.51-2.46 (m, 2H), 2.06-1.85 (m, 3H), 1.01-0.88 (m, 2H), 0.88-0.69 (m, 2H). |
| | methyl (3R,5R)-3-(cyanomethyl)-5-(1- | | |
| | cyclopropyl-3-(2-fluoro-4- | | |
| | (trifluoromethoxy)benzyl)ureido)pipe | | |
| | ridine-1-carboxylate | | |
| Example 187 | 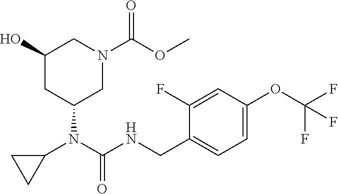 [SMILES: COC(=O)N1C[C@H](O)C[C@@H](N(C(=O)NCc2ccc(OC(F)(F)F)cc2F)C2CC2)C1] | 450.2 | 1H NMR (400 MHz, MeOD) δ 7.42 (t, J = 8.4 Hz, 1H), 7.09 (t, J = 9.3 Hz, 2H), 6.91 (t, J = 5.8 Hz, 1H), 4.43 (s, 2H), 3.99 (t, J = 22.7 Hz, 4H), 3.68 (s, 3H), 3.35 (s, 1H), 2.97-2.80 (m, 1H), 2.59 (s, 1H), 2.46-2.32 (m, 1H), 1.88 (d, J = 13.3 Hz, 1H), 1.01-0.88 (m, 2H), 0.77 (s, 2H). |
| | methyl (3R,5R)-3-(1-cyclopropyl-3-(2- | | |
| | fluoro-4- | | |
| | (trifluoromethoxy)benzyl)ureido)-5- | | |
| | hydroxypiperidine-1-carboxylate | | |
| Example 188 (cis) | 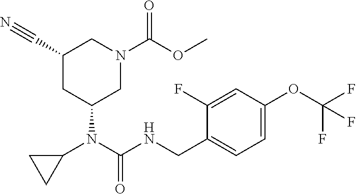 [SMILES: COC(=O)N1C[C@@H](C#N)C[C@@H](N(C(=O)NCc2ccc(OC(F)(F)F)cc2F)C2CC2)C1] | 459.1 | 1H NMR (400 MHz, MeOD) δ 7.43 (t, J = 8.6 Hz, 1H), 7.09 (t, J = 8.7 Hz, 2H), 6.98 (t, J = 5.8 Hz, 0.65H), 4.43 (s, 2H), 4.33 (s, 1H), 4.02 (d, J = 10.5 Hz, 1H), 3.70 (s, 3H), 3.57 (s, 1H), 3.28 (s, 1H), 2.89 (d, J = 8.2 Hz, 2H), 2.58 (s, 1H), 2.44 (q, J = 12.2 Hz, 1H), 2.23 (d, J = 12.1 Hz, 1H), 1.00-0.89 |
| | methyl (3S,5R)-3-cyano-5-(1- | | (m, 2H), 0.77 (s, 2H). |
| | cyclopropyl-3-(2-fluoro-4- | | |
| | (trifluoromethoxy)benzyl)ureido)pipe | | |
| | ridine-1-carboxylate | | |
| Example 189 | 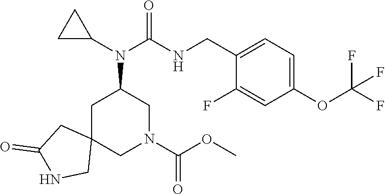 [SMILES: COC(=O)N1C[C@H](N(C(=O)NCc2ccc(OC(F)(F)F)cc2F)C2CC2)CC2(CNC(=O)C2)C1] | 503.3 | 1H NMR (400 MHz, MeOD) δ 7.43 (t, J = 8.6 Hz, 1H), 7.09 (t, J = 8.3 Hz, 2H), 7.01-6.92 (m, 1H), 4.49-4.39 (m, 2H), 4.17-3.98 (m, 2H), 3.83-3.72 (m, 1H), 3.69 (d, J = 1.8 Hz, 3H), 3.29-3.21 (m, 2H), 3.21- 3.10 (m, 2H), 2.78- 2.60 (m, 1H), 2.60-2.48 (m, 1H), 2.29-2.20 (m, |
| | methyl (9R)-9-(1-cyclopropyl-3-(2- | | 3H), 2.17-2.06 (m, 1H), |
| | fluoro-4- | | 1.97-1.80 (m, 1H), 1.00- |
| | (trifluoromethoxy)benzyl)ureido)-3- | | 0.90 (m, 2H), 0.82-0.70 |
| | oxo-2,7-diazaspiro[4.5]decane-7- | | (m, 2H). |
| | carboxylate | | |
| Example 190 (cis) | 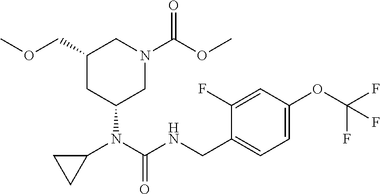 [SMILES: COC[C@H]1C[C@@H](N(C(=O)NCc2ccc(OC(F)(F)F)cc2F)C2CC2)CN(C(=O)OC)C1] | 478.1 | 1H NMR (400 MHz, CD3OD) δ 7.42 (t, J = 8.6 Hz, 1H), 7.08 (t, J = 8.8 Hz, 2H), 6.93 (t, J = 5.6 Hz, 1H), 4.43 (d, J = 4.5 Hz, 2H), 4.20- 3.98 (m, 2H), 3.67 (s, 4H), 3.31-3.22 (m, 5H), 3.17 (t, J = 12.0 Hz, 1H), 2.49 (d, J = 36.7 Hz, 2H), 1.91-1.76 (m, 3H), 0.93 (d, J = 6.0 Hz, 2H), 0.74 |
| | methyl (3R,5S)-3-(1-cyclopropyl-3-(2- | | (s, 2H). |
| | fluoro-4- | | |
| | (trifluoromethoxy)benzyl)ureido)-5- | | |
| | (methoxymethyl)piperidine-1- | | |
| | carboxylate | | |
| Example 191 (cis) | 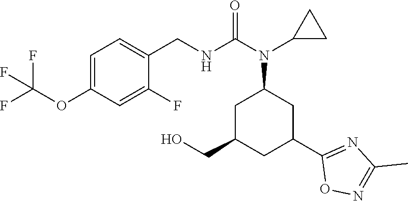 [SMILES: Cc1noc(C2C[C@@H](CO)C[C@@H](N(C(=O)NCc3ccc(OC(F)(F)F)cc3F)C3CC3)C2)n1] | 488.3 | 1H NMR (400 MHz, MeOD) δ 7.43 (t, J = 8.6 Hz, 1H), 7.09 (t, J = 8.6 Hz, 2H), 6.97 (t, J = 5.8 Hz, 1H), 4.44 (d, J = 5.8 Hz, 2H), 4.15 (dd, J = 13.2, 3.6 Hz, 1H), 4.01 (dd, J = 12.4, 4.2 Hz, 1H), 3.83-3.74 (m, 1H), 3.54- 3.42 (m, 3H), 2.81-2.72 (m, 1H), 2.61-2.54 (m, 1H), 2.15 (s, 3H), 1.99-1.83 (m, 3H), 0.96 (dd, J = 5.8, |
| | 1-cyclopropyl-3-(2-fluoro-4- | | 3.4 Hz, 2H), 0.82-0.74 |
| | (trifluoromethoxy)benzyl)-1-((3R,5S)- | | (m, 2H). |
| | 5-(hydroxymethyl)-1-(3-methyl-1,2,4- | | |
| | oxadiazol-5-yl)piperidin-3-yl)urea | | |
| Example 192 (cis) | 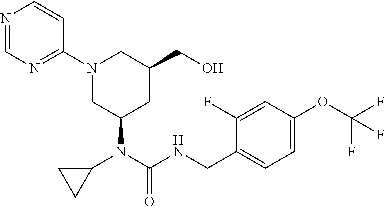 [SMILES: O=C(NCc1ccc(OC(F)(F)F)cc1F)N(C1CC1)[C@@H]1C[C@H](CO)CN(c2ccncn2)C1] | 484.3 | 1H NMR (400 MHz, MeOD) δ 8.46 (s, 1H), 8.09 (d, J = 6.6 Hz, 1H), 7.45 (t, J = 8.6 Hz, 1H), 7.10 (t, J = 8.0 Hz, 2H), 6.88-6.84 (m, 1H), 4.62 (s, 1H), 4.46 (d, J = 4.7 Hz, 3H), 3.73 (s, 1H), 3.56- 3.46 (m, 2H), 3.29 (s, 1H), 2.68-2.55 (m, 2H), 2.00 (dd, J = 20.6, 8.7 Hz, 2H), 1.78 (d, J = 6.8 Hz, 1H), 1.00-0.93 (m, 2H), 0.80 |
| | 1-cyclopropyl-3-(2-fluoro-4- | | (d, J = 3.6 Hz, 2H). |
| | (trifluoromethoxy)benzyl)-1-((3R,5S)- | | |
| | 5-(hydroxymethyl)-1-(pyrimidin-4- | | |
| | yl)piperidin-3-yl)urea | | |
| Example 193 (cis) | 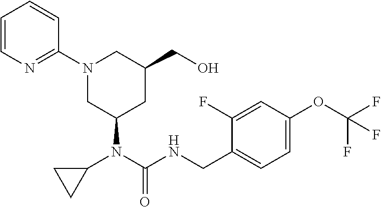 [SMILES: O=C(NCc1ccc(OC(F)(F)F)cc1F)N(C1CC1)[C@@H]1C[C@H](CO)CN(c2ccccn2)C1] | 483.3 | 1H NMR (400 MHz, Methanol-d4) δ 8.07-7.99 (m, 1H), 7.55-7.49 (m, 1H), 7.48-7.42 (m, 1H), 7.15-7.04 (m, 2H), 7.03- 6.94 (m, 1H), 6.90-6.82 (m, 1H), 6.66-6.57 (m, 1H), 4.51-4.43 (m, 2H), 4.40-4.29 (m, 1H), 4.31- 4.21 (m, 1H), 3.90-3.76 (m, 1H), 3.57-3.43 (m, 2H), 3.24-3.12 (m, 1H), 2.63-2.50 (m, 2H), 2.01- |
| | 1-cyclopropyl-3-(2-fluoro-4- | | 1.88 (m, 2H), 1.87-1.76 |
| | (trifluoromethoxy)benzyl)-1-((3R,5S)- | | (m, 1H), 1.01-0.90 (m, |
| | 5-(hydroxymethyl)-1-(pyridin-2- | | 2H), 0.85-0.75 (m, 2H). |
| | yl)piperidin-3-yl)urea | | |
| Example 194 (cis) | 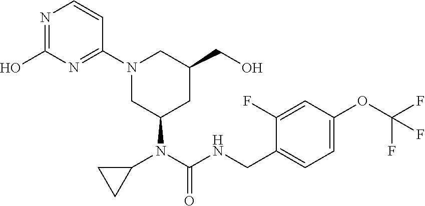 [SMILES: O=C(NCc1ccc(OC(F)(F)F)cc1F)N(C1CC1)[C@@H]1C[C@H](CO)CN(c2ccnc(O)n2)C1] | 500.3 | 1H NMR (400 MHz, MeOD) δ 7.51-7.35 (m, 2H), 7.15-7.03 (m, 2H), 7.02-6.93 (m, 1H), 6.19 (d, J = 7.3 Hz, 1H), 5.27- 4.87 (m, 1H), 4.44 (s, 2H), 4.31-3.87 (m, 1H), 3.87- 3.56 (m, 1H), 3.56-3.41 (m, 2H), 3.37-3.31 (m, 1H), 2.86-2.36 (m, 2H), 2.09-1.83 (m, 2H), 1.82- 1.64 (m, 1H), 1.02-0.90 (m, 2H), 0.89-0.65 (m, |
| | 1-cyclopropyl-3-(2-fluoro-4- | | 2H). |
| | (trifluoromethoxy)benzyl)-1-((3R,5S)- | | |
| | 5-(hydroxymethyl)-1-(2- | | |
| | hydroxypyrimidin-4-yl)piperidin-3- | | |
| | yl)urea | | |
| Example 195 (cis) | 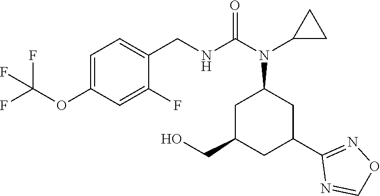 [SMILES: O=C(NCc1ccc(OC(F)(F)F)cc1F)N(C1CC1)[C@H]1CC(c2ncon2)C[C@@H](CO)C1] | 474.2 | 1HNMR (400 MHz, MeOD) δ 8.72 (s, 1H), 7.44 (t, J = 8.5 Hz, 1H), 7.09 (t, J = 9.0 Hz, 0 H), 6.94 (t, J = 5.8 Hz, 1H), 4.44 (d, J = 5.6 Hz, 2H), 4.10 (dd, J = 13.2, 2.9 Hz, 1H), 3.95 (dd, J = 12.1, 4.3 Hz, 1H), 3.81 (dd, J = 9.7, 5.6 Hz, 1H), 3.47 (qd, J = 11.0, 5.7 Hz, 2H), 3.36 (d, J = 11.8 Hz, 1H), 2.66-2.53 (m, 2H), 1.97- |
| | 1-cyclopropyl-3-(2-fluoro-4- | | 1.81 (m, 3H), 1.01-0.91 |
| | (trifluoromethoxy)benzyl)-1-((3R,5S)- | | (m, 2H), 0.78 (ddd, J = 9.1, |
| | 5-(hydroxymethyl)-1-(1,2,4-oxadiazol- | | 6.0, 4.4 Hz, 2H). |
| | 3-yl)piperidin-3-yl)urea | | |
| Example 196 (cis) | 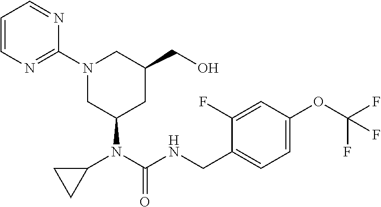 [SMILES: O=C(NCc1ccc(OC(F)(F)F)cc1F)N(C1CC1)[C@@H]1C[C@H](CO)CN(c2ncccn2)C1] | 484.2 | 1H NMR (400 MHz, MeOD) δ 8.29 (d, J = 4.8 Hz, 2H), 7.44 (t, J = 8.5 Hz, 1H), 7.09 (t, J = 9.3 Hz, 2H), 6.94 (t, J = 5.8 Hz, 1H), 6.55 (t, J = 4.8 Hz, 1H), 4.82 (d, J = 3.7 Hz, 1H), 4.72-4.65 (m, 1H), 4.45 (d, J = 5.6 Hz, 2H), 3.70 (ddd, J = 15.4, 11.0, 4.4 Hz, 1H), 3.52-3.46 (m, 2H), 3.26 (d, J = |
| | 1-cyclopropyl-3-(2-fluoro-4- | | 11.9 Hz, 1H), 2.56 (ddd, |
| | (trifluoromethoxy)benzyl)-1-((3R,5S)- | | J = 28.6, 15.6, 7.7 Hz, 2H), |
| | 5-(hydroxymethyl)-1-(pyrimidin-2- | | 1.95 (dd, J = 14.4, 8.2 Hz, |
| | yl)piperidin-3-yl)urea | | 2H), 1.81-1.69 (m, 1H), |
| | | | 0.99-0.90 (m, 2H), 0.80 |
| | | | (ddd, J = 12.6, 9.5, 4.0 Hz, |
| | | | 2H). |
| Example 197 (cis) | 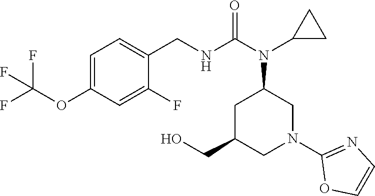 [SMILES: O=C(NCc1ccc(OC(F)(F)F)cc1F)N(C1CC1)[C@@H]1C[C@H](CO)CN(c2ncco2)C1] | 473.3 | 1H NMR (400 MHz, MeOD) δ 7.48-7.40 (m, 1H), 7.36 (s, 1H), 7.09 (t, J = 8.7 Hz, 2H), 6.80 (s, 1H), 4.44 (s, 2H), 4.14-4.07 (m, 1H), 4.01-3.94 (m, 1H), 3.87-3.76 (m, 1H), 3.53-3.41 (m, 2H), 3.39- 3.32 (m, 1H), 2.68-2.60 (m, 1H), 2.59-2.53 (m, 1H), 1.94-1.82 (m, 3H), 1.01-0.91 (m, 2H), 0.81- |
| | 1-cyclopropyl-3-(2-fluoro-4- | | 0.73 (m, 2H). |
| | (trifluoromethoxy)benzyl)-1-((3R,5S)- | | |
| | 5-(hydroxymethyl)-1-(oxazol-2- | | |
| | yl)piperidin-3-yl)urea | | |
| Example 198 (cis) | 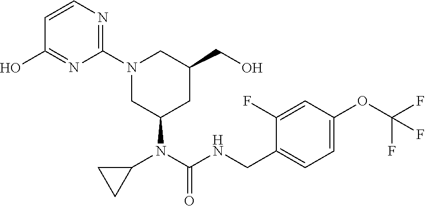 [SMILES: O=C(NCc1ccc(OC(F)(F)F)cc1F)N(C1CC1)[C@@H]1C[C@H](CO)CN(c2nccc(O)n2)C1] | 500.1 | 1H NMR (400 MHz, CD3OD) δ 7.59 (s, 1H), 7.43 (d, J = 8.3 Hz, 1H), 7.09 (t, J = 7.7 Hz, 2H), 5.74 (d, J = 7.0 Hz, 1H), 4.42 (d, J = 16.3 Hz, 3H), 4.26 (s, 1H), 3.74 (s, 1H), 3.53-3.46 (m, 2H), 3.28- 3.26 (m, 1H), 2.68-2.50 (m, 2H), 1.95 (d, J = 8.6 Hz, 2H), 1.81 (s, 1H), 0.96 (s, 2H), 0.79 (s, 2H). |
| | 1-cyclopropyl-3-(2-fluoro-4- | | |
| | (trifluoromethoxy)benzyl)-1-((3R,5S)- | | |
| | 5-(hydroxymethyl)-1-(4- | | |
| | hydroxypyrimidin-2-yl)piperidin-3- | | |
| | yl)urea | | |
| Example 199 (cis) | 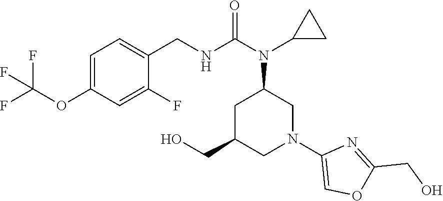 [SMILES: O=C(NCc1ccc(OC(F)(F)F)cc1F)N(C1CC1)[C@@H]1C[C@H](CO)CN(c2coc(CO)n2)C1] | 503.2 | 1H NMR (400 MHz, MeOD) δ 7.44 (t, J = 8.6 Hz, 1H), 7.09 (t, J = 8.7 Hz, 2H), 6.96 (t, J = 5.9 Hz, 1H), 6.70-6.63 (m, 1H), 4.46-4.42 (m, 4H), 4.17-4.07 (m, 1H), 4.01- 3.95 (m, 1H), 3.88-3.75 (m, 1H), 3.54-3.42 (m, 2H), 3.41-3.33 (m, 1H), 2.68-2.59 (m, 1H), 2.59- 2.51 (m, 1H), 1.97-1.79 (m, 3H), 0.99-0.92 (m, |
| | 1-cyclopropyl-3-(2-fluoro-4- | | 2H), 0.82-0.75 (m, 2H). |
| | (trifluoromethoxy)benzyl)-1-((3R,5S)- | | |
| | 5-(hydroxymethyl)-1-(2- | | |
| | (hydroxymethyl)oxazol-4-yl)piperidin- | | |
| | 3-yl)urea | | |
| Example 200 | 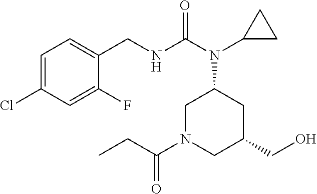 [SMILES: CCC(=O)N1C[C@@H](CO)C[C@@H](N(C(=O)NCc2ccc(Cl)cc2F)C2CC2)C1] | 412.2 | 1HNMR (400 MHz, MeOD) δ 7.32 (q, J = 8.2 Hz, 1H), 7.16 (dd, J = 9.9, 4.3 Hz, 2H), 4.64 (d, J = 9.1 Hz, 1H), 4.48 (d, J = 12.5 Hz, 1H), 4.44-4.34 (m, 2H), 4.02 (d, J = 14.0 Hz, 1H), 3.91-3.75 (m, 1H), 3.62- 3.36 (m, 3H), 3.24 (t, J = 12.0 Hz, 1H), 3.06 (t, J = 11.9 Hz, 1H), 2.76-2.65 (m, 1H), 2.54 (dtd, J = |
| | 3-(4-chloro-2-fluorobenzyl)-1- | | 18.3, 6.7, 3.3 Hz, 1H), |
| | cyclopropyl-1-((3R,5S)-5- | | 2.47-2.37 (m, 2H), 2.24 |
| | (hydroxymethyl)-1- | | (t, J = 12.3 Hz, 1H), |
| | propionylpiperidin-3-yl)urea | | 1.97-1.81 (m, 2H), 1.80- |
| | | | 1.59 (m, 1H), 1.11 (td, |
| | | | J = 7.5, 4.8 Hz, 3H), 0.94 |
| | | | (ddd, J = 13.9, 7.6, 4.4 Hz, |
| | | | 2H), 0.84-0.66 (m, 2H). |
| Example 201 | 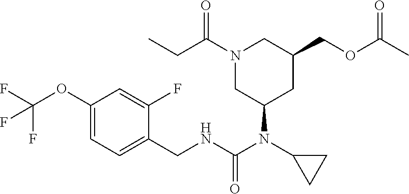 [SMILES: CCC(=O)N1C[C@@H](COC(C)=O)C[C@@H](N(C(=O)NCc2ccc(OC(F)(F)F)cc2F)C2CC2)C1] | 504.3 | 1H NMR (400 MHz, MeOD) δ 7.49-7.37 (m, 1H), 7.09 (t, J = 8.7 Hz, 2H), 6.96 (dt, J = 19.0, 5.6 Hz, 1H), 4.60 (t, J = 9.4 Hz, 1H), 4.5-4.38 (m, 2H), 4.04 (dd, J = 11.1, 5.0 Hz, 1H), 3.99-3.84 (m, 2H), 3.83-3.73 (m, 1H), 3.58-3.47 (m, 1H), 3.26 (s, 1H), 3.09 (t, J = 11.9 Hz, 1H), 2.83-2.73 |
| | ((3S,5R)-5-(1-cyclopropyl-3-(2-fluoro- | | (m, 1H), 2.55 (dtd, J = 14.1, |
| | 4-(trifluoromethoxy)benzyl)ureido)-1- | | 6.7, 3.5 Hz, 1H), 2.48- |
| | propionylpiperidin-3-yl)methyl | | 2.36 (m, 2H), 2.28 (t, J = |
| | acetate | | 12.1 Hz, 1H), 2.05 (s, 3H), |
| | | | 2.03-1.83 (m, 3H), 1.11 |
| | | | (td, J = 7.4, 3.7 Hz, 3H), |
| | | | 1.01-0.88 (m, 2H), 0.76 |
| | | | (ddd, J = 18.3, 16.3, |
| | | | 9.8 Hz, 2H). |
| Example 202 | 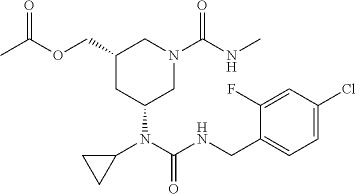 [SMILES: CNC(=O)N1C[C@@H](COC(C)=O)C[C@@H](N(C(=O)NCc2ccc(Cl)cc2F)C2CC2)C1] | 455.2 | 1H NMR (400 MHz, CD3OD) δ 7.32 (t, J = 8.3 Hz, 1H), 7.21-7.13 (m, 2H), 4.41 (d, J = 5.6 Hz, 2H), 4.09 (d, J = 13.3 Hz, 1H), 4.04-3.91 (m, 2H), 3.86 (d, J = 16.1 Hz, 1H), 3.65 (s, 1H), 3.18-3.12 (m, 1H), 2.71 (s, 3H), 2.59- 2.50 (m, 1H), 2.48-2.37 (m, 1H), 2.06 (s, 3H), 1.90 (d, J = 4.9 Hz, 3H), 0.95 (d, |
| | ((3S,5R)-5-(3-(4-chloro-2- | | J = 3.3 Hz, 2H), 0.76 (s, |
| | fluorobenzyl)-1-cyclopropylureido)-1- | | 2H). |
| | (methylcarbamoyl)piperidin-3- | | |
| | yl)methyl acetate | | |
| Example 203 | 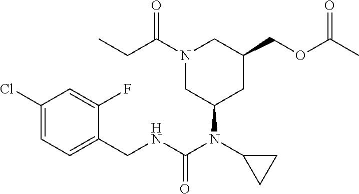 [SMILES: CCC(=O)N1C[C@@H](COC(C)=O)C[C@@H](N(C(=O)NCc2ccc(Cl)cc2F)C2CC2)C1] | 454.1 | 1HNMR (400 MHz, MeOD) δ 7.37-7.27 (m, 1H), 7.16 (dd, J = 7.7, 4.8 Hz, 2H), 6.92 (dt, J = 18.9, 5.8 Hz, 1H), 4.61 (d, J = 10.6 Hz, 1H), 4.48 (d, J = 10.0 Hz, 1H), 4.44-4.34 (m, 2H), 4.04 (dd, J = 11.1, 5.0 Hz, 1H), 3.95 (dd, J = 11.1, 6.9 Hz, 1H), 3.87 (d, J = 13.1 Hz, 1H), 3.78 (dt, J = 11.4, 5.8 Hz, 1H), 3.52 (t, J = 3.9 Hz, 1H), 3.27 (d, |
| | ((3S,5R)-5-(3-(4-chloro-2- | | J = 12.6 Hz, 1H), 3.09 (t, |
| | fluorobenzyl)-1-cyclopropylureido)-1- | | J = 11.9 Hz, 1H), 2.78 (dd, |
| | propionylpiperidin-3-yl)methyl | | J = 13.3, 11.2 Hz, 1H), |
| | acetate | | 2.54 (dtd, J = 14.1, 6.7, |
| | | | 3.5 Hz, 1H), 2.47-2.36 |
| | | | (m, 2H), 2.28 (t, J = |
| | | | 12.1 Hz, 1H), 2.06 (s, 3H), |
| | | | 2.04-1.84 (m, 3H), 1.11 |
| | | | (td, J = 7.5, 3.1 Hz, 3H), |
| | | | 1.00-0.89 (m, 2H), 0.84- |
| | | | 0.65 (m, 2H). |
| Example 204 (cis) | 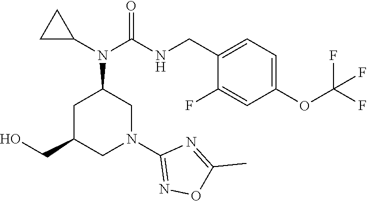 [SMILES: Cc1nc(N2C[C@@H](CO)C[C@@H](N(C(=O)NCc3ccc(OC(F)(F)F)cc3F)C3CC3)C2)no1] | 488.2 | 1H NMR (400 MHz, MeOD) δ 7.44 (t, J = 8.5 Hz, 1H), 7.09 (t, J = 9.3 Hz, 2H), 6.94 (t, J = 5.8 Hz, 1H), 4.45 (d, J = 4.8 Hz, 2H), 4.08-3.98 (m, 1H), 3.92-3.75 (m, 2H), 3.53-3.39 (m, 2H), 3.27 (s, 1H), 2.62-2.51 (m, 2H), 2.43 (s, 3H), 1.97- 1.75 (m, 3H), 1.03-0.89 (m, 2H), 0.84-0.71 (m, |
| | 1-cyclopropyl-3-(2-fluoro-4- | | 2H). |
| | (trifluoromethoxy)benzyl)-1-((3R,5S)- | | |
| | 5-(hydroxymethyl)-1-(5-methyl-1,2,4- | | |
| | oxadiazol-3-yl)piperidin-3-yl)urea | | |
| Example 205 (cis) | 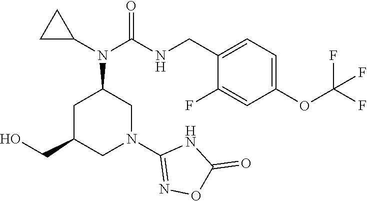 [SMILES: O=C(NCc1ccc(OC(F)(F)F)cc1F)N(C1CC1)[C@@H]1C[C@H](CO)CN(c2noc(=O)[nH]2)C1] | 490.2 | 1H NMR (400 MHz, MeOD) δ 7.43 (t, J = 8.6 Hz, 1H), 7.09 (t, J = 7.6 Hz, 2H), 6.99 (s, 1H), 4.44 (d, J = 2.7 Hz, 2H), 3.85 (d, J = 4.6 Hz, 1H), 3.69 (d, J = 13.8 Hz, 1H), 3.56-3.47 (m, 2H), 3.43 (dd, J = 11.0, 5.9 Hz, 1H), 3.26 (t, J = 11.9 Hz, 1H), 2.64-2.51 (m, 2H), 1.97-1.83 (m, 3H), 0.96 (d, J = 6.4 Hz, 2H), 0.77 |
| | 1-cyclopropyl-3-(2-fluoro-4- | | (d, J = 3.4 Hz, 2H). |
| | (trifluoromethoxy)benzyl)-1-((3R,5S)- | | |
| | 5-(hydroxymethyl)-1-(5-oxo-4,5- | | |
| | dihydro-1,2,4-oxadiazol-3- | | |
| | yl)piperidin-3-yl)urea | | |
| Example 206 (cis) | 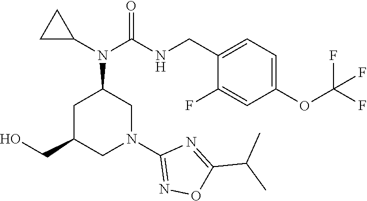 [SMILES: CC(C)c1nc(N2C[C@@H](CO)C[C@@H](N(C(=O)NCc3ccc(OC(F)(F)F)cc3F)C3CC3)C2)no1] | 516.4 | 1H NMR (400 MHz, MeOD) δ 7.44 (t, J = 8.4 Hz, 1H), 7.09 (t, J = 9.0 Hz, 2H), 6.95 (t, J = 5.9 Hz, 1H), 4.45 (d, J = 5.8 Hz, 2H), 4.13-4.01 (m, 1H), 3.91 (dd, J = 11.9, 4.2 Hz, 1H), 3.81 (d, J = 4.3 Hz, 1H), 3.55-3.40 (m, 2H), 3.27 (s, 1H), 3.16- 3.02 (m, 1H), 2.62-2.50 (m, 2H), 1.90 (d, J = 7.9 Hz, 3H), 1.33 (d, J = |
| | 1-cyclopropyl-3-(2-fluoro-4- | | 7.0 Hz, 6H), 1.02-0.89 |
| | (trifluoromethoxy)benzyl)-1-((3R,5S)- | | (m, 2H), 0.79 (dd, J = 7.1, |
| | 5-(hydroxymethyl)-1-(5-isopropyl- | | 3.6 Hz, 2H). |
| | 1,2,4-oxadiazol-3-yl)piperidin-3- | | |
| | yl)urea | | |
| Example 207 (cis) | 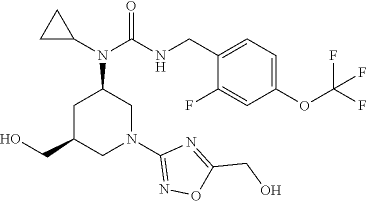 [SMILES: O=C(NCc1ccc(OC(F)(F)F)cc1F)N(C1CC1)[C@@H]1C[C@H](CO)CN(c2noc(CO)n2)C1] | 504.2 | 1H NMR (400 MHz, DMSO) δ 7.42 (t, J = 8.6 Hz, 1H), 7.33 (d, J = 10.4 Hz, 1H), 7.23 (d, J = 8.5 Hz, 1H), 6.96 (t, J = 5.9 Hz, 1H), 5.87 (t, J = 6.4 Hz, 1H), 4.63 (t, J = 5.3 Hz, 1H), 4.55 (d, J = 6.4 Hz, 2H), 4.32 (d, J = 5.7 Hz, 2H), 3.94 (d, J = 11.4 Hz, 1H), 3.75 (d, J = 9.1 Hz, 2H), 3.30- 3.22 (m, 2H), 3.15 (d, J = |
| | 1-cyclopropyl-3-(2-fluoro-4- | | 12.0 Hz, 1H), 2.46 (d, |
| | (trifluoromethoxy)benzyl)-1-((3R,5S)- | | J = 3.4 Hz, 2H), 1.74 |
| | 5-(hydroxymethyl)-1-(5- | | (dd, J = 18.3, 7.6 Hz, |
| | (hydroxymethyl)-1,2,4-oxadiazol-3- | | 3H), 0.89 (d, J = 6.6 Hz, |
| | yl)piperidin-3-yl)urea | | 2H), 0.67 (d, J = 11.1 Hz, |
| | | | 2H). |
| Example 208 (cis) | 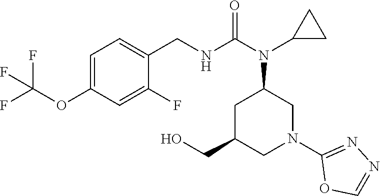 [SMILES: O=C(NCc1ccc(OC(F)(F)F)cc1F)N(C1CC1)[C@@H]1C[C@H](CO)CN(c2nnco2)C1] | 474.2 | 1HNMR(400 MHz,MeOD) δ 8.35 (s, 1H), 7.44 (t, J = 8.6 Hz,1H), 7.09 (t, J = 8.4 Hz, 2H), 6.98 (t, J = 5.9 Hz, 1H), 4.44 (d, J = 5.0 Hz, 2H), 4.10-4.02 (m, 1H), 3.92 (dd, J = 12.0, 4.1 Hz, 1H), 3.88-3.77 (m, 1H), 3.55-3.41 (m, 3H), 2.81-2.71 (m, 1H), 2.61- 2.53 (m, 1H), 2.00-1.81 (m, 3H), 0.96 (dd, J = 6.4, 2.7 Hz, 2H), 0.83-0.74 |
| | 1-cyclopropyl-3-(2-fluoro-4- | | (m, 2H). |
| | (trifluoromethoxy)benzyl)-1-((3R,5S)- | | |
| | 5-(hydroxymethyl)-1-(1,3,4-oxadiazol- | | |
| | 2-yl)piperidin-3-yl)urea | | |
| Example 209 (cis) | 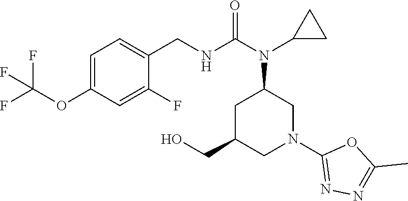 [SMILES: Cc1nnc(N2C[C@@H](CO)C[C@@H](N(C(=O)NCc3ccc(OC(F)(F)F)cc3F)C3CC3)C2)o1] | 488.2 | 1H NMR (400 MHz, MeOD) δ 7.43 (t, J = 8.6 Hz, 1H), 7.09 (t, J = 8.4 Hz, 2H), 4.44 (s, 2H), 4.07-3.93 (m, 1H), 3.84 (dd, J = 14.2, 11.6 Hz, 2H), 3.52 (dd, J = 11.0, 4.4 Hz, 1H), 3.48-3.37 (m, 2H), 2.76-2.67 (m, 1H), 2.60- 2.52 (m, 1H), 2.37 (s, 3H), 1.93 (t, J = 8.6 Hz, 3H), 1.00-0.91 (m, 2H), 0.82- 0.73 (m, 2H). |
| | 1-cyclopropyl-3-(2-fluoro-4- | | |
| | (trifluoromethoxy)benzyl)-1-((3R,5S)- | | |
| | 5-(hydroxymethyl)-1-(5-methyl-1,3,4- | | |
| | oxadiazol-2-yl)piperidin-3-yl)urea | | |
| Example 210 (cis) | 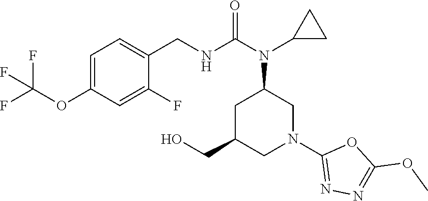 [SMILES: COc1nnc(N2C[C@@H](CO)C[C@@H](N(C(=O)NCc3ccc(OC(F)(F)F)cc3F)C3CC3)C2)o1] | 504.1 | 1H NMR (400 MHz, MeOD) δ 7.43 (t, J = 8.6 Hz, 1H), 7.09 (t, J = 8.6 Hz, 2H), 6.97 (t, J = 5.8 Hz, 1H), 4.44 (s, 2H), 4.06 (s, 3H), 3.87 (t, J = 12.7 Hz, 2H), 3.75 (dd, J = 12.1, 4.5 Hz, 1H), 3.53-3.42 (m, 2H), 3.36 (d, J = 11.8 Hz, 1H), 2.68- 2.61 (m, 1H), 0.59- 2.5 3 (m, 1H), 1.91 (d, J = 5.2 Hz, 3H), 1.00- |
| | 1-cyclopropyl-3-(2-fluoro-4- | | 0.92 (m, 2H), 0.81-0.73 |
| | (trifluoromethoxy)benzyl)-1-((3R,5S)- | | (m, 2H). |
| | 5-(hydroxymethyl)-1-(5-methoxy- | | |
| | 1,3,4-oxadiazol-2-yl)piperidin-3- | | |
| | yl)urea | | |
| Example 211 (cis) | 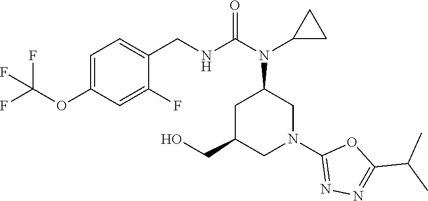 [SMILES: CC(C)c1nnc(N2C[C@@H](CO)C[C@@H](N(C(=O)NCc3ccc(OC(F)(F)F)cc3F)C3CC3)C2)o1] | 516.3 | 1H NMR (400 MHz, MeOD) δ 7.44 (t, J = 8.6 Hz, 1H), 7.09 (t, J = 8.4 Hz, 2H), 6.98 (t, J = 5.9 Hz, 1H), 4.44 (d, J = 5.3 Hz, 2H), 4.05- 3.96 (m, 1H), 3.87 (dd, J = 12.3, 3.4 Hz, 2H), 3.52 (dd, J = 11.0, 4.2 Hz, 1H), 3.47-3.37 (m, 2H), 3.05 (dt, J = 13.9, 7.0 Hz, 1H), 2.75-2.67 (m, 1H), 2.60-2.54 (m, |
| | 1-cyclopropyl-3-(2-fluoro-4- | | 1H), 1.92 (d, J = 6.7 Hz, |
| | (trifluoromethoxy)benzyl)-1-((3R,5S)- | | 3H), 1.32 (d, J = 7.0 Hz, |
| | 5-(hydroxymethyl)-1-(5-isopropyl- | | 6H), 0.99-0.91 (m, 2H), |
| | 1,3,4-oxadiazol-2-yl)piperidin-3- | | 0.82-0.74 (m, 2H). |
| | yl)urea | | |
| Example 212 (cis) | 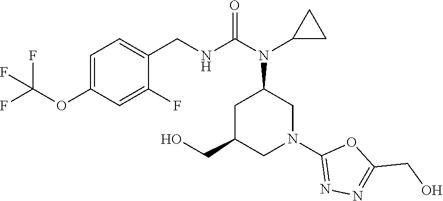 [SMILES: O=C(NCc1ccc(OC(F)(F)F)cc1F)N(C1CC1)[C@@H]1C[C@H](CO)CN(c2nnc(CO)o2)C1] | 504.3 | 1HNMR (400 MHz, MeOD) δ 7.44 (t, J = 8.6 Hz, 1H), 7.09 (t, J = 8.6 Hz, 2H), 6.98 (t, J = 5.9 Hz, 1H), 4.56 (s, 2H), 4.44 (d, J = 5.4 Hz, 2H), 4.09-4.02 (m, 1H), 3.91 (dd, J = 11.9, 4.2 Hz, 1H), 3.85 (d, J = 4.5 Hz, 1H), 3.53 (dd, J = 11.0, 4.4 Hz, 1H), 3.49-3.4 1 (m, 2H), 2.82-2.70 (m, 1H), 2.60- 2.52 (m, 1H), 1.93 (d, |
| | 1-cyclopropyl-3-(2-fluoro-4- | | J = 7.6 Hz, 3H), 0.96 |
| | (trifluoromethoxy)benzyl)-1-((3R,5S)- | | (td, J = 6.4, 3.2 Hz, 2H), |
| | 5-(hydroxymethyl)-1-(5- | | 0.85-0.73 (m, 2H). |
| | (hydroxymethyl)-1,3,4-oxadiazol-2- | | |
| | yl)piperidin-3-yl)urea | | |
| Example 213 (cis) | 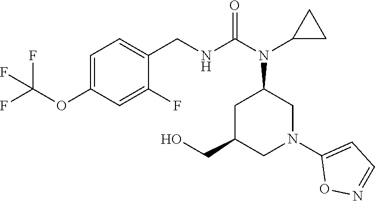 [SMILES: O=C(NCc1ccc(OC(F)(F)F)cc1F)N(C1CC1)[C@@H]1C[C@H](CO)CN(c2ccno2)C1] | 473.2 | 1H NMR (400 MHz, MeOD) δ 8.05 (d, J = 2.1 Hz, 1H), 7.44 (t, J = 8.6 Hz, 1H), 7.09 (t, J = 8.6 Hz, 2H), 5.20 (d, J = 2.1 Hz, 1H), 4.45 (d, J = 4.3 Hz, 2H), 3.89 (d, J = 12.7 Hz, 2H), 3.73 (dd, J = 12.1, 4.3 Hz, 1H), 3.52 (dd, J = 11.0, 3.9 Hz, 1H), 3.44 (dd, J = 11.0, 6.1 Hz, 1H), 3.34 (s, 1H), 2.69-2.61 (m, |
| | 1-cyclopropyl-3-(2-fluoro-4- | | 1H), 2.59-2.52 (m, 1H), |
| | (trifluoromethoxy)benzyl)-1-((3R,5S)- | | 1.94-1.86 (m, 3H), 0.96 |
| | 5-(hydroxymethyl)-1-(isoxazol-5- | | (d, J = 6.6 Hz, 2H), 0.77 |
| | yl)piperidin-3-yl)urea | | (dd, J = 10.7, 3.6 Hz, 2H). |
| Example 214 (cis) | 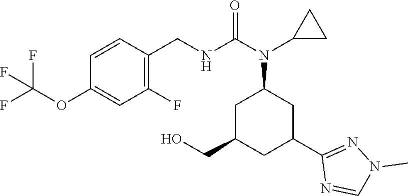 [SMILES: Cn1cnc(C2C[C@@H](CO)C[C@@H](N(C(=O)NCc3ccc(OC(F)(F)F)cc3F)C3CC3)C2)n1] | 487.3 | 1H NMR (400 MHz, MeOD) δ 7.98 (s, 1H), 7.44 (t, J = 8.5 Hz, 1H), 7.09 (t, J = 8.7 Hz, 2H), 6.93 (t, J = 5.8 Hz, 1H), 4.44 (d, J = 5.7 Hz, 2H), 4.05 (d, J = 12.3 Hz, 1H), 3.93 (dd, J = 11.8, 4.1 Hz, 1H), 3.85 (d, J = 4.1 Hz, 1H), 3.73 (s, 3H), 3.47 (d, J = 4.0 Hz, 2H), 3.20 (t, J = 11.5 Hz, 1H), 2.60-2.52 |
| | 1-cyclopropyl-3-(2-fluoro-4- | | (m, 1H), 2.46 (dd, J = 12.5, |
| | (trifluoromethoxy)benzyl)-1-((3R,5S)- | | 10.7 Hz, 1H), 1.95-1.81 |
| | 5-(hydroxymethyl)-1-(1-methyl-1H- | | (m, 3H), 0.99-0.89 (m, |
| | 1,2,4-triazol-3-yl)piperidin-3-yl)urea | | 2H), 0.84-0.71 (m, 2H). |
| Example 215 (cis) | 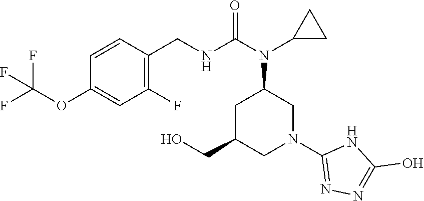 [SMILES: O=C(NCc1ccc(OC(F)(F)F)cc1F)N(C1CC1)[C@@H]1C[C@H](CO)CN(c2nnc(O)[nH]2)C1] | 489.3 | 1H NMR (400 MHz, MeOD) δ 7.43 (t, J = 8.5 Hz, 1H), 7.09 (t, J = 7.8 Hz, 2H), 4.44 (d, J = 3.3 Hz, 2H), 3.88 (s, 1H), 3.71 (d, J = 12.8 Hz, 1H), 3.58 (d, J = 12.0 Hz, 1H), 3.49-3.40 (m, 2H), 3.13 (t, J = 11.6 Hz, 1H), 2.57- 2.43 (m, 2H), 1.96-1.81 (m, 3H), 0.95 (d, J = 4.4 Hz, 2H), 0.77 (d, J = 2.5 Hz, 0H). |
| | 1-cyclopropyl-3-(2-fluoro-4- | | |
| | (trifluoromethoxy)benzyl)-1-((3R,5S)- | | |
| | 1-(5-hydroxy-4H-1,2,4-triazol-3-yl)-5- | | |
| | (hydroxymethyl)piperidin-3-yl)urea | | |
| Example 216 (cis) | 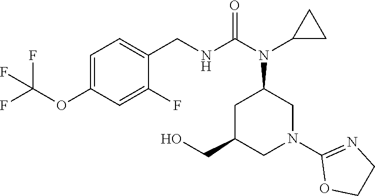 [SMILES: O=C(NCc1ccc(OC(F)(F)F)cc1F)N(C1CC1)[C@@H]1C[C@H](CO)CN(C2=NCCO2)C1] | 475.1 | 1H NMR (400 MHz, MeOD) δ 7.43 (t, J = 8.6 Hz, 1H), 7.09 (t, J = 8.2 Hz, 2H), 4.43 (s, 2H), 4.34 (t, J = 8.6 Hz, 2H), 3.99 (d, J = 12.9 Hz, 1H), 3.86 (dd, J = 12.2, 3.9 Hz, 1H), 3.72 (t, J = 8.6 Hz, 3H), 3.50-3.36 (m, 2H), 3.24 (t, J = 11.8 Hz, 1H), 2.59-2.42 (m, 2H), 1.84 (dd, J = 19.8, 8.8 Hz, 3H), |
| | 1-cyclopropyl-1-((3R,5S)-1-(4,5- | | 1.02-0.89 (m, 2H), 0.84- |
| | dihydrooxazol-2-yl)-5- | | 0.68 (m, 2H). |
| | (hydroxymethyl)piperidin-3-yl)-3-(2- | | |
| | fluoro-4- | | |
| | (trifluoromethoxy)benzyl)urea | | |
| Example 217 (cis) | 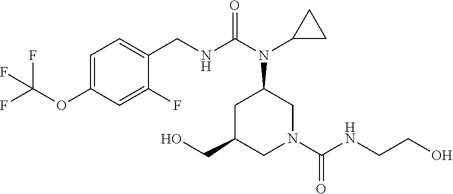 [SMILES: O=C(NCCO)N1C[C@@H](CO)C[C@@H](N(C(=O)NCc2ccc(OC(F)(F)F)cc2F)C2CC2)C1] | 493.3 | 1H NMR (400 MHz, Methanol-d4) δ 7.50-7.38 (m, 1H), 7.15-7.03 (m, 2H), 4.50-4.37 (m, 2H), 4.17-4.05 (m, 1H), 3.96- 3.87 (m, 1H), 3.77-3.64 (m, 1H), 3.63-3.52 (m, 2H), 3.52-3.37 (m, 2H), 3.29-3.23 (m, 2H), 3.19- 3.06 (m, 1H), 2.59-2.48 (m, 1H), 2.46-2.34 (m, 1H), 1.96-1.79 (m, 2H), |
| | (3R,5S)-3-(1-cyclopropyl-3-(2-fluoro- | | 1.77-1.65 (m, 1H), 1.02- |
| | 4-(trifluoromethoxy)benzyl)ureido)-N- | | 0.90 (m, 2H), 0.81-0.71 |
| | (2-hydroxyethyl)-5- | | (m, 2H). |
| | (hydroxymethyl)piperidine-1- | | |
| | carboxamide | | |
| Example 218 (cis), | 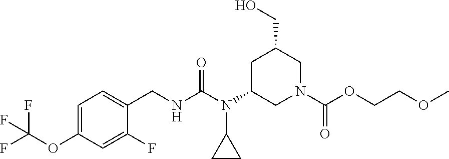 [SMILES: COCCOC(=O)N1C[C@@H](CO)C[C@@H](N(C(=O)NCc2ccc(OC(F)(F)F)cc2F)C2CC2)C1] | 508.3 | 1H NMR (400 MHz, MeOD) δ 7.43 (t, J = 8.6 Hz, 1H), 7.09 (t, J = 8.4 Hz, 2H), 4.43 (s, 2H), 4.26-4.17 (m, 3H), 4.06 (d, J = 8.6 Hz, 1H), 3.66 (s, 1H), 3.61-3.58 (m, 2H), 3.50-3.39 (m, 2H), 3.36 (s, 3H), 3.25-3.16 (m, 1H), 2.55 (s, 1H), 2.43 |
| | 2-methoxyethyl (3R,5S)-3-(1- | | (s, 1H), 1.8 7 (d, J = 8.4 Hz, |
| | cyclopropyl-3-(2-fluoro-4- | | 2H), 1.74 (s, 1H), 0.94 |
| | (trifluoromethoxy)benzyl)ureido)-5- | | (d, J = 3.2 Hz, 2H), 0.76 |
| | (hydroxymethyl)piperidine-1- | | (d, J = 4.1 Hz, 2H). |
| | carboxylate | | |
| Example 219 (cis), | 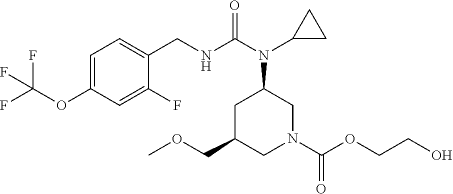 [SMILES: COC[C@H]1C[C@@H](N(C(=O)NCc2ccc(OC(F)(F)F)cc2F)C2CC2)CN(C(=O)OCCO)C1] | 508.3 | 1H NMR (400 MHz, MeOD) δ 7.43 (t, J = 8.6 Hz, 1H), 7.09 (t, J = 8.5 Hz, 2H), 4.44 (s, 2H), 4.26-4.17 (m, 1H), 4.16- 4.03 (m, 3H), 3.72 (t, J = 4.8 Hz, 3H), 3.32 (s, 3H), 3.27 (dd, J = 12.5, 6.8 Hz, 2H), 3.16 (d, J = 11.4 Hz, 1H), 2.50 (d, J = 33.2 Hz, 2H), 1.96-1.78 (m, 3H), 1.00-0.86 (m, 2H), 0.75 |
| | 2-hydroxyethyl (3R,5S)-3-(1- | | (s, 2H). |
| | cyclopropyl-3-(2-fluoro-4- | | |
| | (trifluoromethoxy)benzyl)ureido)-5- | | |
| | (methoxymethyl)piperidine-1- | | |
| | carboxylate | | |
| Example 220 (cis) | 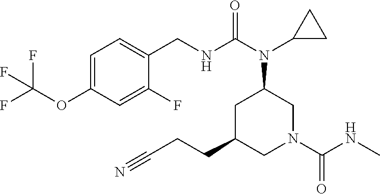 [SMILES: CNC(=O)N1C[C@@H](CCC#N)C[C@@H](N(C(=O)NCc2ccc(OC(F)(F)F)cc2F)C2CC2)C1] | 486.1 | 1HNMR(400 MHz,MeOD) δ 7.43 (t, J = 8.4 Hz, 1H), 7.09 (t, J = 7.9 Hz, 2H), 4.43 (s, 2H), 4.00 (d, J = 13.2 Hz, 1H), 3.88 (d, J = 12.7 Hz, 1H), 3.64 (t, J = 11.8 Hz, 1H), 3.16 (d, J = 11.7 Hz, 1H), 2.71 (s, 3H), 2.53 (dd, J = 13.0, 6.3 Hz, 3H), 2.39-2.30 (m, 1H), 1.98 (d, J = 11.9 Hz, 1H), 1.81 (d, J = 11.7 Hz, |
| | (3S,5R)-3-(2-cyanoethyl)-5-(1- | | 1H), 1.70-1.51 (m, 3H), |
| | cyclopropyl-3-(2-fluoro-4- | | 0.95 (s, 2H), 0.77 (s, 2H). |
| | (trifluoromethoxy)benzyl)ureido)-N- | | |
| | methylpiperidine-1-carboxamide | | |
| Example 221 (cis) | 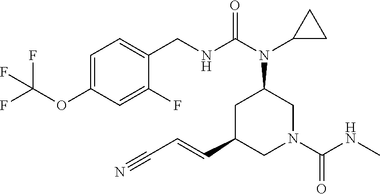 [SMILES: CNC(=O)N1C[C@@H](/C=C/C#N)C[C@@H](N(C(=O)NCc2ccc(OC(F)(F)F)cc2F)C2CC2)C1] | 484.2 | 1H NMR (400 MHz, MeOD) δ 7.43 (dd, J = 11.3, 5.9 Hz, 1H), 7.09 (t, J = 7.5 Hz, 2H), 6.69 (dd, J = 16.5, 7.0 Hz, 1H), 6.46- 6.37 (m, 1H), 5.64 (dd, J = 16.5, 1.2 Hz, 1H), 5.57 (d, J = 11.0 Hz, 1H), 4.43 (s, 2H), 4.02 (d, J = 12.9 Hz, 1H), 3.91-3.82 (m, 1H), 3.66 (ddd, J = 11.8, 7.8, 4.2 Hz, 1H), 3.28-3.12 |
| | (3R,5R)-3-((E)-2-cyanovinyl)-5-(1- | | (m, 2H), 2.71 (d, J = |
| | cyclopropyl-3-(2-fluoro-4- | | 5.5 Hz, 3H), 2.60-2.52 |
| | (trifluoromethoxy)benzyl)ureido)-N- | | (m, 2H), 2.52-2.41 (m, |
| | methylpiperidine-1-carboxamide | | 1H), 2.19-1.87 (m, 2H), |
| | | | 0.95 (td, J = 6.5, 2.9 Hz, |
| | | | 2H), 0.83-0.70 (m, 2H). |
| Example 223 (rac) | 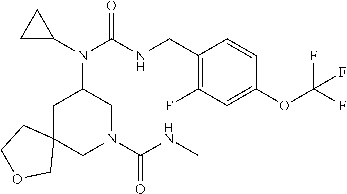 [SMILES: CNC(=O)N1CC(N(C(=O)NCc2ccc(OC(F)(F)F)cc2F)C2CC2)CC2(CCOC2)C1] | 489.3 | 1H NMR (400 MHz, MeOD) δ 7.51-7.38 (m, 1H), 7.18-6.98 (m, 2H), 4.52-4.38 (m, 2H), 4.03- 3.77 (m, 5H), 3.66-3.54 (m, 1H), 3.48-3.37 (m, 1H), 3.14 (t, J = 11.7 Hz, 1H), 2.72 (s, 3H), 2.64- 2.50 (m, 2H), 2.36-2.13 (m, 1H), 2.01-1.82 (m, 1H), 1.80-1.63 (m, 2H), 1.04-0.89 (m, 2H), 0.84- |
| | 9-(1-cyclopropyl-3-(2-fluoro-4- | | 0.69 (m, 2H). |
| | (trifluoromethoxy)benzyl)ureido)-N- | | |
| | methyl-2-oxa-7-azaspiro[4.5]decane-7- | | |
| | carboxamide | | |
| Example 224 (cis) | 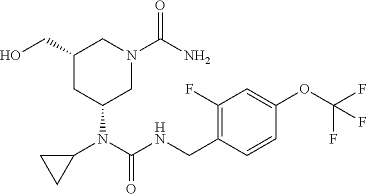 [SMILES: NC(=O)N1C[C@@H](CO)C[C@@H](N(C(=O)NCc2ccc(OC(F)(F)F)cc2F)C2CC2)C1] | 449.1 | 1H NMR (400 MHz, MeOD) δ 7.43 (t, J = 8.6 Hz, 1H), 7.08 (t, J = 7.8 Hz, 2H), 6.95 (t, J = 6.1 Hz, 1H), 4.43 (s, 2H), 4.09 (d, J = 13.2 Hz, 1H), 3.91 (d, J = 12.3 Hz, 1H), 3.69 (dt, J = 15.6, 5.5 Hz, 1H), 3.52-3.39 (m, 2H), 3.22-3.10 (m, 1H), 2.54 (td, J = 6.6, 3.4 Hz, 1H), 2.47- |
| | (3R,5S)-3-(1-cyclopropyl-3-(2-fluoro- | | 2.37 (m, 1H), 1.94-1.80 |
| | 4-(trifluoromethoxy)benzyl)ureido)-5- | | (m, 2H), 1.80-1.66 (m, |
| | (hydroxymethyl)piperidine-1- | | 1H), 0.99-0.89 (m, 2H), |
| | carboxamide | | 0.80-0.70 (m, 2H). |

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound of Formula (I): 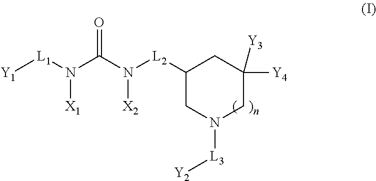
wherein:
n is 0, 1, or 2;
L₁ is absent or selected from -alkyl-, -hydroxyalkyl-, -cycloalkyl-, and -heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cyanoalkyl, —O-alkoxyalkyl, —O-haloalkyl, —O-hydroxyalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, haloalkyl, and cycloalkyl;
each Y₂″ is independently alkyl, or both instances taken together with the nitrogen atom to which they are bonded form a 4-, 5- or 6-membered heterocyclyl or cycloalkyl; and
Y₃ and Y₄ together with the carbon to which they are bonded form a 4-, 5-, or 6-membered cycloalkyl, cycloheteroalkyl, or heterocyclyl, or Y₃ and Y₄ are each independently selected from —OH, —CN, —CO₂H, —CO₂(alkyl), alkyl, hydroxyalkyl, cyanoalkyl, and halogen;
or a pharmaceutically acceptable salt thereof.

2. The compound of claim 1, wherein
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl; and
Y₃ and Y₄ together with the carbon to which they are bonded form a 4-, 5-, or 6-membered cycloalkyl, cycloheteroalkyl, or heterocyclyl.

3. The compound of claim 1 or 2, wherein Y₃ and Y₄ together with the carbon to which they are bonded form an unsubstituted 4-, 5-, or 6-membered heterocyclyl.

4. The compound of any one of claims 1-3, wherein Y₃ and Y₄ together with the carbon to which they are bonded form an unsubstituted 4-, 5-, or 6-membered cyclic urea, cyclic carbamate, cyclic sulfone, cyclic sulfonamide, lactam, azalactam, or lactone.

5. The compound of claim 3 or 4, wherein Y₃ and Y₄ together with the carbon to which they are bonded form any one of the following structures: 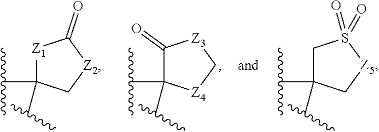
wherein
Z₁ is selected from O, NH, and CH₂;
Z₂ is selected from O, NH, and CH₂;
Z₃ is selected from O and NH;
Z₄ is selected from NH and CH₂; and
Z₅ is selected from NH and CH₂;
provided that one of Z₁ and Z₂ is not CH₂.

6. The compound of claim 3 or 4, wherein Y₃ and Y₄ together with the carbon to which they are bonded form the following structure: 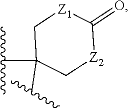
wherein
Z₁ is selected from O, NH, and CH₂; and
Z₂ is selected from O, NH, and CH₂;
provided that one of Z₁ and Z₂ is not CH₂.

7. The compound of claim 3 or 4, wherein Y₃ and Y₄ together with the carbon to which they are bonded form any one of the following structures: 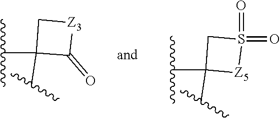
wherein
Z₃ is selected from O and NH; and
Z₅ is selected from NH and CH₂.

8. The compound of any one of claims 1-3, wherein Y₃ and Y₄ together with the carbon to which they are bonded form an unsubstituted 4-, 5-, or 6-membered cycloheteroalkyl.

9. The compound of claim 8, wherein Y₃ and Y₄ together with the carbon to which they are bonded form an unsubstituted piperidinyl, tetrahydrofuranyl, azetidinyl, or morpholinyl.

10. The compound of any one of claims 1-9, wherein Y₃ and Y₄ together with the carbon to which they are bonded form any one of the following structures:

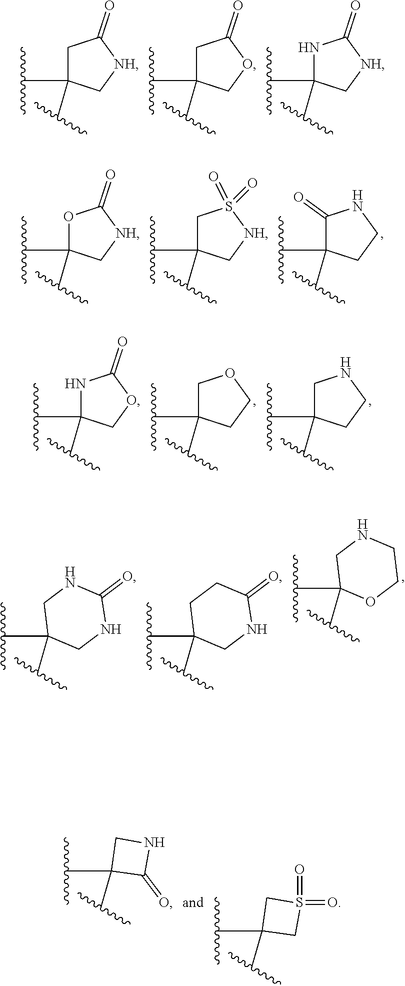

11. The compound of claim 1 or 2, wherein Y₃ and Y₄ together with the carbon to which they are bonded form a substituted 4-, 5-, or 6-membered heterocyclyl.

12. The compound of any one of claims 1-3, wherein Y₃ and Y₄ together with the carbon to which they are bonded form a substituted 4-, 5-, or 6-membered cyclic urea, cyclic carbamate, cyclic sulfone, cyclic sulfonamide, lactam, azalactam, or lactone.

13. The compound of claim 12, wherein the cyclic urea, cyclic carbamate, cyclic sulfonamide, lactam, or azalactam is N-substituted.

14. The compound of claim 13, wherein the cyclic urea, cyclic carbamate, cyclic sulfonamide, lactam, or azalactam is N-alkyl substituted.

15. The compound of any one of claims 11-14, wherein Y₃ and Y₄ together with the carbon to which they are bonded form the following structure: 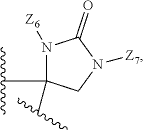
wherein
Z₆ is selected from —H and alkyl; and
Z₇ is selected from —H and alkyl;
provided that Z₆ and Z₇ are not both —H.

16. The compound of any one of claims 11-14, wherein Y₃ and Y₄ together with the carbon to which they are bonded form any one the following structures: 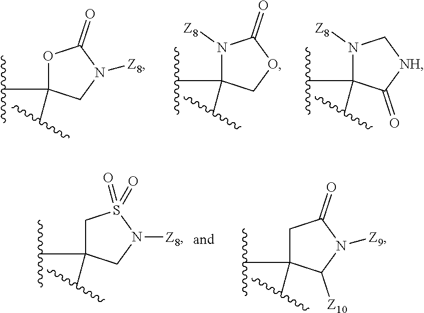
wherein
each Z₈ is independently an alkyl;
Z₉ is selected from —H and alkyl; and
Z₁₀ is selected from —H and alkyl;
provided that Z₉ and Z₁₀ are not both —H.

17. The compound of any one of claims 11-14, wherein Y₃ and Y₄ together with the carbon to which they are bonded form any one of the following structures: 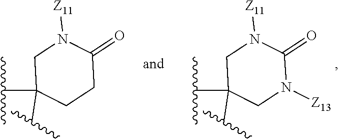
wherein
Z₁₁ is alkyl;
Z₁₂ is selected from —H and alkyl; and
Z₁₃ is selected from —H and alkyl;
provided that Z₁₂ and Z₁₃ are not both —H.

18. The compound of any one of claims 11-14, wherein Y₃ and Y₄ together with the carbon to which they are bonded form the following structure: 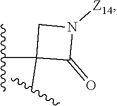
wherein Z₁₄ is alkyl.

19. The compound of any one of claims 1-32, wherein Y₃ and Y₄ together with the carbon to which they are bonded form a substituted 4-, 5-, or 6-membered cycloheteroalkyl

20. The compound of claim 19, wherein Y₃ and Y₄ together with the carbon to which they are bonded form a substituted piperidinyl, tetrahydrofuranyl, azetidinyl, or morpholinyl.

21. The compound of claim 19 or 20, wherein Y₃ and Y₄ together with the carbon to which they are bonded form an N-alkyl or N-acetyl substituted piperidinyl, azetidinyl, or morpholinyl.

22. The compound of any one of claims 1-3, wherein Y₃ and Y₄ together with the carbon to which they are bonded form a substituted 4-, 5-, or 6-membered cycloalkyl.

23. The compound of claim 22, wherein Y₃ and Y₄ together with the carbon to which they are bonded form a substituted cyclopropyl or cyclobutyl.

24. The compound of any one of claims 11-23, wherein Y₃ and Y₄ together with the carbon to which they are bonded form any one of the following structures:

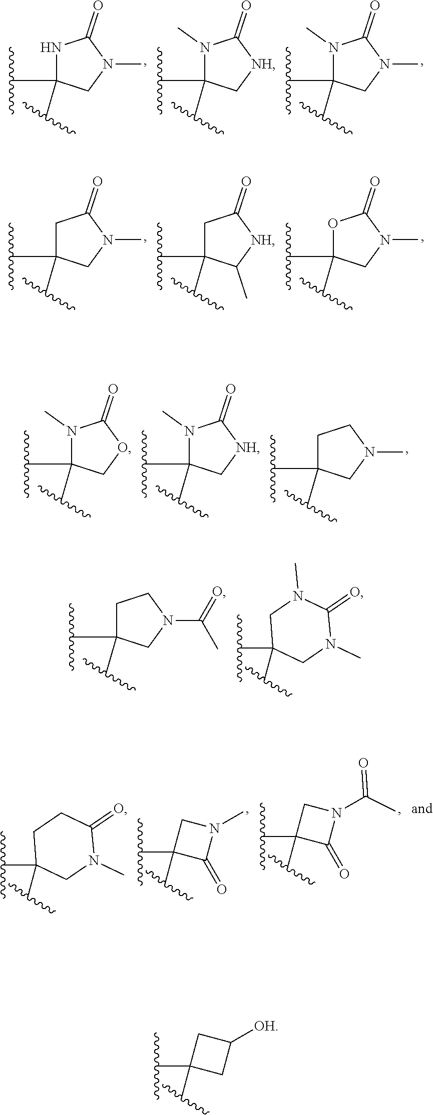

25. The compound of claim 1, wherein Y₃ and Y₄ together with the carbon to which they are bonded form an unsubstituted 4-, 5-, or 6-membered cycloheteroalkyl.

26. The compound of claim 25, wherein Y₃ and Y₄ together with the carbon to which they are bonded form a substituted tetrahydrofuranyl or tetrahydropyranyl.

27. The compound of claim 25 or 26 wherein Y₃ and Y₄ together with the carbon to which they are bonded form any one of the following structures:

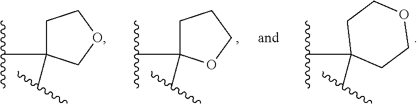

28. The compound of claim 1, wherein Y₃ and Y₄ are each independently selected from —OH, —CN, —CO₂H, —CO₂(alkyl), alkyl, hydroxyalkyl, cyanoalkyl, and halogen;

29. The compound of claim 27, wherein Y₃ and Y₄ are each independently selected from —F, —OH, —CN, —CO₂H, —CO₂Et, —CH₃, —CH₂CH₃, —CH₂CN, —CH₂OH, and —CH₂OSO₂Me.

30. The compound of claim 28, wherein Y₃ is selected from —F, CH₃, —CH₂CH₃; and Y₄ is is selected from —OH, —CN, —CO₂H, —CO₂Et, —CH₂CN, —CH₂OH, and —CH₂OSO₂Me.

31. A compound of Formula (II): 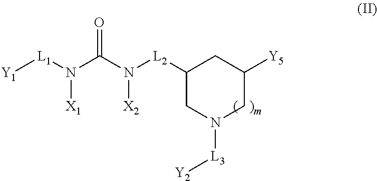
wherein:
m is 0, 1, or 2;
L₁ is absent or selected from -alkyl-, -hydroxyalkyl-, -cycloalkyl-, -heteroaryl-, and -heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cyanoalkyl, —O-alkoxyalkyl, —O-haloalkyl, —O-hydroxyalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, haloalkyl, and cycloalkyl;
each Y₂″ is independently alkyl, or both instances taken together with the nitrogen atom to which they are bonded form a 4-, 5- or 6-membered heterocyclyl or cycloalkyl;
Y₅ is selected from cycloalkyl, heteroaryl, heterocyclyl, C₀-C₆ alkyl-Y₅′, and C₂-C₆ alkenyl-Y₅′;
Y₅′ is selected from —CN, —OH, —NH₂, —OSO₂-alkyl, —NH(Y₅″), —C(O)N(Y₅′″)₂, —SO₂N(Y₅′″)₂, —O(CO)—Y₅′″, —(CO)O—Y₅′″, alkoxy, benzyloxy, —C═N—O(alkyl), and a squaramide moiety;
Y₅″ is selected from alkyl, —C(O)-alkyl, and —SO₂-alkyl; and
Y₅′″ is independently for each occurrence selected from —H, alkyl, aminoalkyl, and aryl;
or a pharmaceutically acceptable salt thereof.

32. The compound of claim 31, wherein
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl;
Y₅ is selected from cyano, cycloalkyl, heteroaryl, heterocyclyl, alkyl-Y₅′, and a squaramide moiety;
Y₅′ is selected from —CN, —OH, —NH₂, —NH(Y₅″), —C(O)N(Y₅′″)₂, —SO₂N(Y₅′″)₂, —and a squaramide moiety;
Y₅″ is selected from alkyl, —C(O)-alkyl, and —SO₂-alkyl; and
Y₅′″ is independently for each occurrence selected from —H and alkyl.

33. The compound of claim 31 or 32 having the structure:

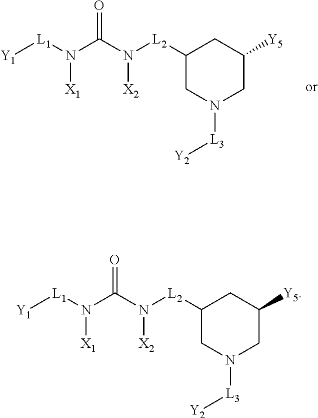

34. The compound of any one of claims 31-33, wherein Y₅ is an unsubstituted 5-membered heteroaryl.

35. The compound of claim 34, wherein Y₅ is selected from an unsubstituted pyrazolyl, unsubstituted diazolyl, unsubstituted oxazolyl, and unsubstituted isooxazolyl.

36. The compound of claim 35, wherein Y₅ is selected from

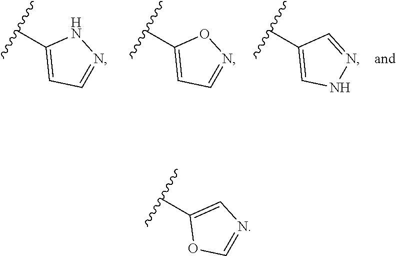 [SMILES: *c1ccn[nH]1 ; *c1ccno1 ; *c1cn[nH]c1 ; "and" ; *c1cnco1]

37. The compound of claim any one of claims 31-33, wherein Y₅ is a substituted 6-membered heteroaryl.

38. The compound of claim 37, wherein Y₅ is selected from a substituted pyridinyl and substituted pyrimidinyl.

39. The compound of claim 38, wherein Y₅ is selected from

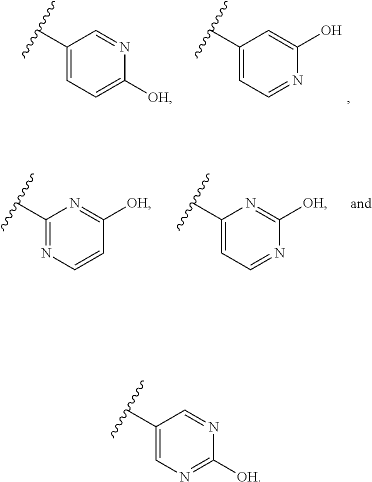 [SMILES: *c1ccc(O)nc1 ; *c1ccnc(O)c1 ; *c1nccc(O)n1 ; *c1ccnc(O)n1 ; "and" ; *c1cnc(O)nc1]

40. The compound of claim any one of claims 31-33, wherein Y₅ is C₀-C₆ alkyl-Y₅′.

41. The compound of claim 40, wherein Y₅ is C₁-C₄ alkyl-Y₅′; and the alkyl is unbranched.

42. The compound of claim 40, wherein Y₅ is C₁-C₄ alkyl-Y₅′; and the alkyl is branched.

43. The compound of claim 40, wherein Y₅ is C₁-C₄ alkyl-Y₅′; and the alkyl is substituted with a cycloalkyl.

44. The compound of any one of claims 40-43, wherein
Y₅′ is selected from —NH(Y₅″), —C(O)N(Y₅′″)₂, and —SO₂N(Y₅′)₂;
Y₅″ is selected from —C(O)—CH₃, and —SO₂—CH₃; and
Y₅′″ is independently for each occurrence selected from —H and —CH₃.

45. The compound of any one of claims 40-43, wherein Y₅′ is —OH, —CN, or alkoxy.

46. The compound of claim 31 or 33, wherein Y₅′ is —O(CO)—Y₅″ or —(CO)O—Y₅′″.

47. The compound of claim 46, wherein Y₅′″ is alkyl, aminoalkyl, or aryl.

48. The compound of any one of claims 40-43, wherein Y₅′ is a squaramide moiety.

49. The compound of claim 48, wherein Y₅′ is 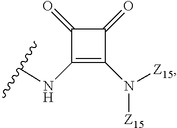
wherein Z₅ is independently for each occurrence selected from —H and alkyl.

50. The compound of claim 49, wherein each Z₁₅ is —H, each Z₁₅ is —CH₃, or one Z₁₅ is —H and the other is —CH₃.

51. The compound of any one of claims 31-33, wherein Y₅ is a squaramide moiety.

52. The compound of claim 51, wherein Y₅ is 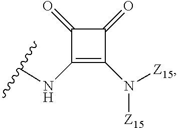
wherein Z₅ is independently for each occurrence selected from —H and alkyl.

53. The compound of claim 52, wherein each Z₁₅ is —H. each Z₁₅ is —CH₃, or one of Z₁₅ is H and the other is —CH₃.

54. The compound of any one of claims 31-53 where Y is selected from

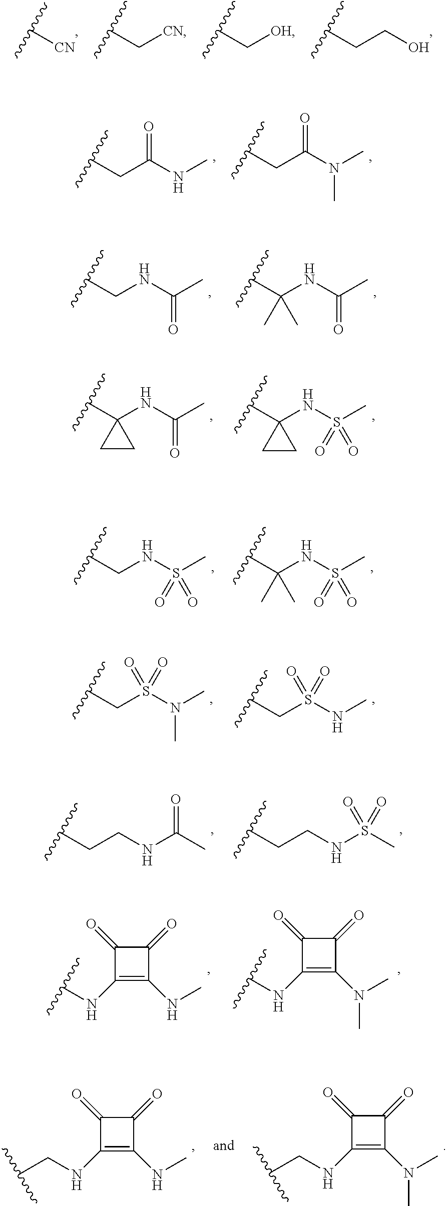

55. The compound of any one of claims 31-47, wherein Y₅ is selected from —OH, —OAc,

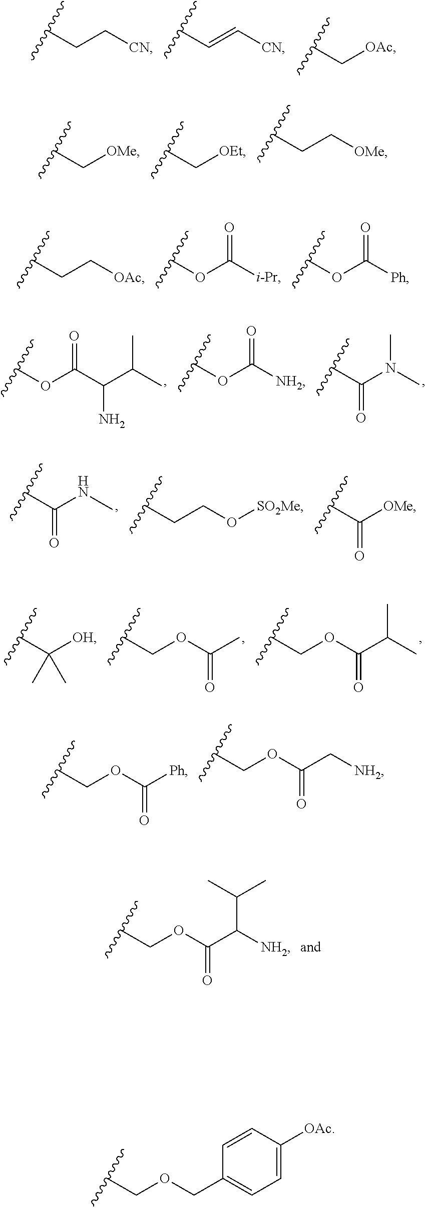

56. The compound of any one of claims 1-55, wherein one of X₁ and X₂ is —H; and the other of X₁ and X₂ is selected from —CH₃, —CH₂CH₃, —CH₂CF₃, —CH₂CH₂CH₃,

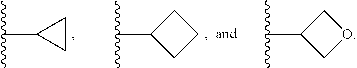

57. The compound of any one of claims 1-55, wherein X₁ is —H; and X₂ is

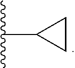 [SMILES: *C1CC1]

58. The compound of any one of claims 1-57, wherein L₁ is absent.

59. The compound of any one of claims 1-58, wherein L₁ is selected from -alkyl-, -hydroxyalkyl-, -cycloalkyl-, -heteroaryl-, and -heteroaryl-CH₂—.

60. The compound of claim 59, wherein L₁ is selected from —CH₂—, —C(H)(CH₃)—, —CH₂CH₂—, and —C(H)(OH)CH₂—.

61. The compound of claim 59, wherein L₁ is selected from

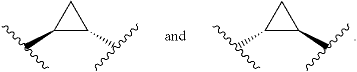 [SMILES: *[C@H]1C[C@@H]1* ; *[C@@H]1C[C@H]1*]

62. The compound of claim 59, wherein L₁ is selected from

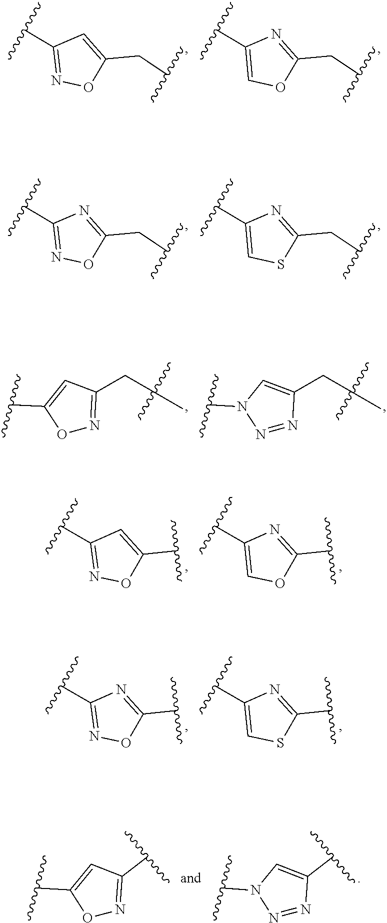

63. The compound of any one of claims 1-62, wherein Y₁ is substituted aryl.

64. The compound of claim 63, wherein Y₁ is 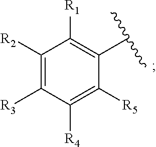
and
R₁, R₂, R₃, R₄, and R₅ are independently selected from —H, halogen, —CN, —CF₃, —CHF₂, —CF₂CH₃, —OCF₃, —OCHF₂, alkyl, haloalkyl, alkenyl, alkynyl, alkoxy, cycloalkyl, heterocyclyl, aryl, and heteroaryl; provided that one of R₁, R₂, R₃, R₄, and R₅ is not —H.

65. The compound of claim 64, wherein R₁, R₂, R₃, R₄, and R₅ are independently selected from —H, —F, —Cl, —Br, —CN, —CH₃, —CH₂CH₃, —CH₂CH₂CH₃, —CH(CH₃)₂, —OCH₃, —OCF₃, and

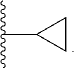 [SMILES: *C1CC1]

66. The compound of any one of claims 64-65, wherein two of R₁, R₂, R₃, R₄, and R₅ are not —H, or three of R₁, R₂, R₃, R₄, and R₅ are not —H.

67. The compound of any one of claims 64-66, wherein Y₁ is selected from

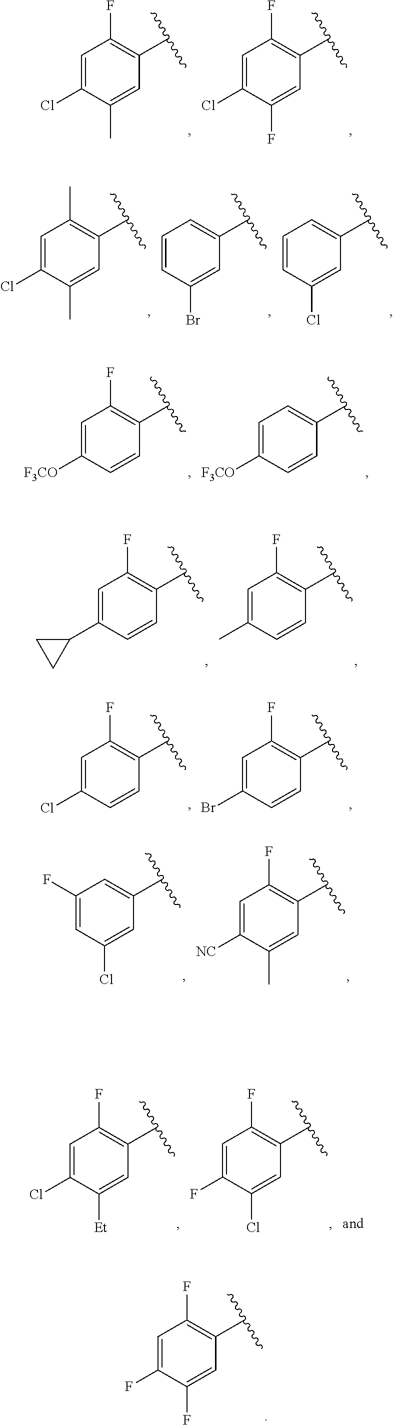

68. The compound of any one of claims 1-62, wherein Y₁ is unsubstituted heteroaryl.

69. The compound of claim 56, wherein Y₁ is selected from

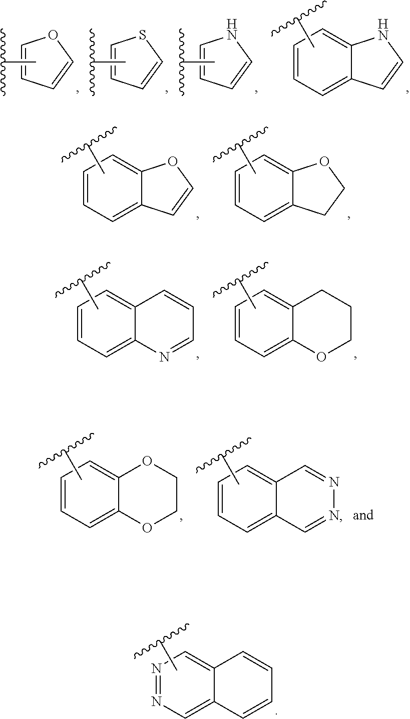

70. The compound of any one of claims 1-62, wherein Y₁ is substituted heteroaryl.

71. The compound of claim 70, wherein Y₁ is selected from 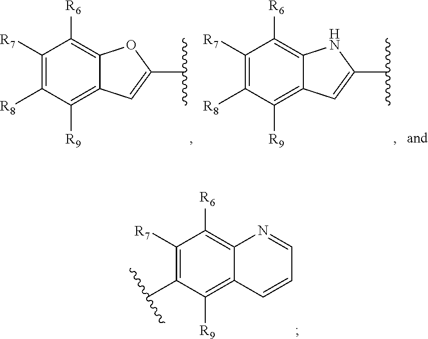
and
each occurrence of R₆, R₇, R₈, and R₉ are independently selected from —H, halogen, —CN, —OCF₃, —OCHF₂, alkyl, haloalkyl, alkenyl, alkynyl, alkoxy, cycloalkyl, aryl, and heteroaryl;
provided that at least one of R₆, R₇, R₈, and R₉ is not —H.

72. The compound of any one of claims 1-71, wherein L₂ is absent.

73. The compound of any one of claims 1-71, wherein L₂ is —CH₂—.

74. The compound of any one of claims 1-73, wherein L₃ is absent.

75. The compound of claim 74, wherein Y₂ is unsubstituted heteroaryl.

76. The compound of claim 75, wherein Y₂ is selected from

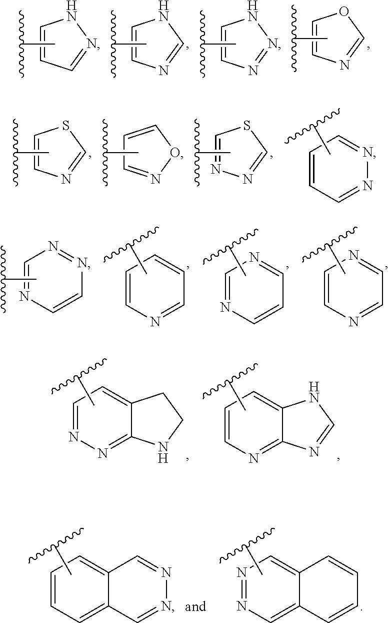

77. The compound of claim 76, wherein Y₂ is

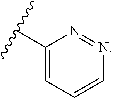 [SMILES: *c1cccnn1]

78. The compound of claim 75, wherein Y₂ is substituted heteroaryl.

79. The compound of claim 78, wherein Y₂ is 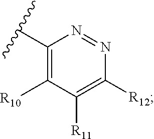
R₁₀, R₁₁, and R₁₂ are independently selected from —H, halogen, —CN, —OH, —NH₂, —OCF₃, —OCHF₂, —OAc, —NHAc, alkyl, haloalkyl, hydroxyalkyl, alkenyl, alkynyl, alkoxy, alkylamino cycloalkyl, aryl, heteroaryl, —C(O)NR₁₃R₁₄, —CO₂R₁₅, and —C(O)NHSO₂R₁₅; provided that at least one of R₁₀, R₁₁, and R₁₂ is not —H; and
each occurrence of R₁₃, R₁₄, and R₁₅ is independently selected from —H, alkyl, aryl, and heteroaryl.

80. The compound of claim 79, wherein R₁₀, R₁₁, and R₁₂ are independently selected from —H, —F, —Cl, —Br, —CN, —CH₃, —CH₂CH₃, —CF₃, —CHF₂, —CF₂CH₃, —OCH₃, —OCF₃, —OCHF₂, —OAc, —NH₂, —NHCH₃, —NHAc, —C(O)NH₂, —C(O)NHCH₃, —C(O)NHCH₂CH₃, —C(O)NHSO₂CH₃, —C(O)NHSO₂CH₂CH₃, —CH₂OH, —CO₂H, phenyl, cyclopropyl, cyclobutyl, imidazolyl, and tetrazolyl.

81. The compound of claim 78, wherein Y₂ is selected from

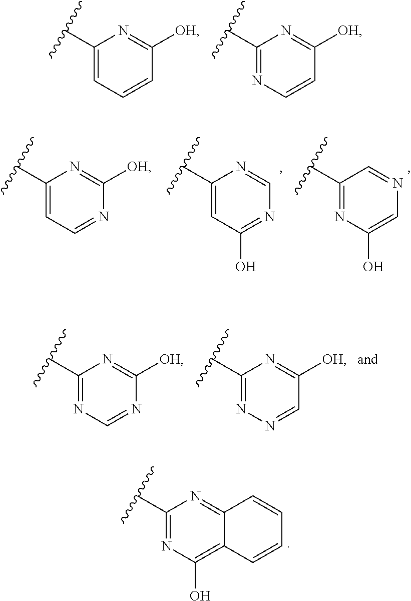

82. The compound of claim 78, wherein Y₂ is 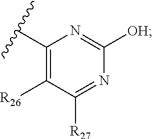
and
R₂₆ and R₂₇ are independently selected from —H, halogen, —CN, —OH, —OCF₃, —OCHF₂, —NH₂, alkyl, alkoxy, alkylamino, and cycloalkyl; provided that at least one of R₆ and R₇ is not —H; or R₆ and R₇ taken together with the carbon atoms to which they are bonded form an unsubstituted or substituted fused C₅-C₇ cycloalkyl; or
Y₂ is 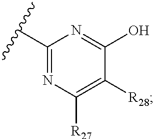
and
R₂₇ and R₂₈ are independently selected from —H, halogen, —CN, —OH, —OCF₃, —OCHF₂, —NH₂, alkyl, alkoxy, alkylamino, and cycloalkyl; provided that at least one of R₇ and R₈ is not —H; or R₇ and R₈ taken together with the carbon atoms to which they are bonded form an unsubstituted or substituted fused C₅-C₇ cycloalkyl; or
Y₂ is 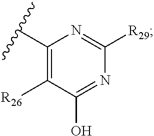
and
R₂₆ and R₂₉ are independently selected from —H, halogen, —CN, —OH, —OCF₃, —OCHF₂, —NH₂, alkyl, alkoxy, alkylamino, and cycloalkyl; provided that at least one of R₆ and R₉ is not —H; or
Y₂ is 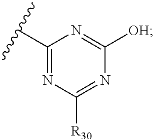
and
R₃₀ is selected from halogen, —CN, —OH, —OCF₃, —OCHF₂, —NH₂, alkyl, alkoxy, alkylamino, and cycloalkyl; or
Y₂ is 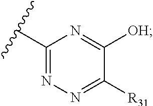
and
R₃₁ is selected from halogen, —CN, —OH, —OCF₃, —OCHF₂, —NH₂, alkyl, alkoxy, alkylamino, and cycloalkyl.

83. The compound of 82, wherein Y₂ is selected from

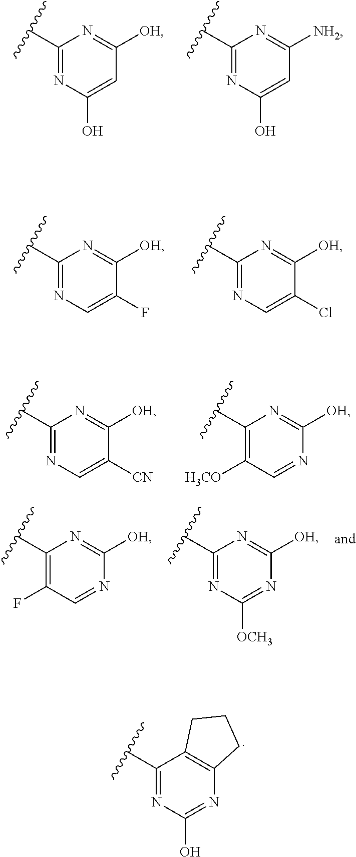

84. The compound of any one of claims 1-73, wherein L₃ is —C(O)—.

85. The compound of claim 84, wherein Y₂ is selected from alkyl, alkenyl, alkynyl, alkoxy, alkoxyalkyl, hydroxyalkyl, haloalkyl, and cyanoalkyl.

86. The compound of claim 85, wherein Y₂ is selected from —CH₃, —CH₂CH₃, —CF₃, —CH₂CH(CH₃)₂, —CH₂CH₂C≡CH, —CH₂CH₂OCH₃, —C(H)(CH₃)CH₂OCH₃, —OCH₃, —OCH₂CH₃, —CH₂OH, —CH₂CH₂OH, —C(CH₃)₂OH, —CH₂CH₂F, —CH₂CH₂CN, and —CH₂OCH₃.

87. The compound of claim 86, wherein Y₂ is selected from —CH₂OH and —CH₂CH₂OH.

88. The compound of claim 84, wherein Y₂ is unsubstituted heteroaryl.

89. The compound of claim 88, wherein Y₂ is

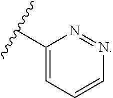 [SMILES: *c1cccnn1]

90. The compound of claim 84, wherein Y₂ is substituted heteroaryl.

91. The compound of claim 90, wherein Y₂ is 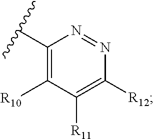
R₁₀, R₁₁, and R₁₂ are independently selected from —H, halogen, —CN, —OH, —NH₂, —OCF₃, —OCHF₂, —OAc, —NHAc, alkyl, haloalkyl, hydroxyalkyl, alkenyl, alkynyl, alkoxy, alkylamino, cycloalkyl, aryl, heteroaryl, —C(O)NR₁₃R₁₄, and —CO₂R₁₅; provided that at least one of R₁₀, R₁₁, and R₁₂ is not —H; and
each occurrence of R₁₃, R₁₄, and R₁₅ is independently selected from —H, alkyl, aryl, and heteroaryl.

92. The compound of claim 91, wherein R₁₀, R₁₁, and R₁₂ are independently selected from —H, —F, —Cl, —Br, —CN, —CH₃, —CH₂CH₃, —CF₃, —CHF₂, —CF₂CH₃, —OCH₃, —OCF₃, —OCHF₂, —OAc, —NH₂, —NHCH₃, —NHAc, —C(O)NH₂, —C(O)NHCH₃, —C(O)NHCH₂CH₃, —C(O)NHSO₂CH₃, —C(O)NHSO₂CH₂CH₃, —CH₂OH, —CO₂H, phenyl, cyclopropyl, cyclobutyl, imidazolyl, and tetrazolyl.

93. The compound of claim 90, wherein Y₂

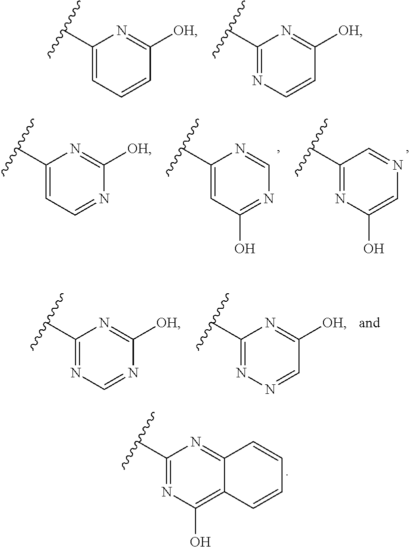

94. The compound of claim 90, wherein Y₂ is 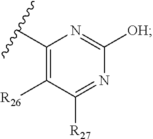
and
R₂₆ and R₂₇ are independently selected from —H, halogen, —CN, —OH, —OCF₃, —OCHF₂, —NH₂, alkyl, alkoxy, alkylamino, and cycloalkyl; provided that at least one of Re and R⁷ is not —H; or R₆ and R₇ taken together with the carbon atoms to which they are bonded form an unsubstituted or substituted fused C₅-C₇ cycloalkyl; or
Y₂ is 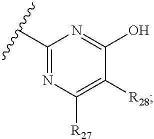
and
R₂₇ and R₂₈ are independently selected from —H, halogen, —CN, —OH, —OCF₃, —OCHF₂, —NH₂, alkyl, alkoxy, alkylamino, and cycloalkyl; provided that at least one of R₇ and R₈ is not —H; or R₇ and R₈ taken together with the carbon atoms to which they are bonded form an unsubstituted or substituted fused C₅-C₇ cycloalkyl; or
Y₂ is 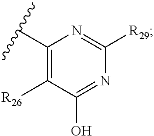
and
R₂₆ and R₂₉ are independently selected from —H, halogen, —CN, —OH, —OCF₃, —OCHF₂, —NH₂, alkyl, alkoxy, alkylamino, and cycloalkyl; provided that at least one of R⁶ and R⁹ is not —H; or
Y₂ is 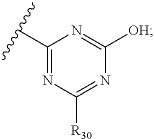
and
R₃₀ is selected from halogen, —CN, —OH, —OCF₃, —OCHF₂, —NH₂, alkyl, alkoxy, alkylamino, and cycloalkyl; or
Y₂ is 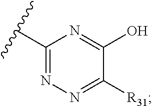
and
R₃₁ is selected from halogen, —CN, —OH, —OCF₃, —OCHF₂, —NH₂, alkyl, alkoxy, alkylamino, and cycloalkyl.

95. The compound of 94, wherein Y₂ is selected from

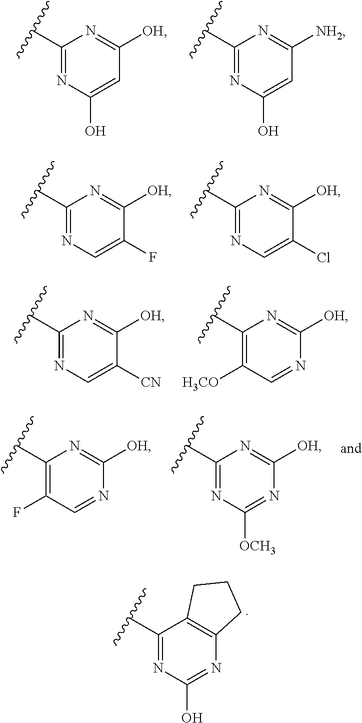

96. The compound of claim 84, wherein Y₂ is —NH(Y₂′).

97. The compound of claim 96, wherein Y₂′ is selected from —H, —OH, alkyl, alkoxy, alkoxyalkyl, and cycloalkyl

98. The compound of claim 97, wherein Y₂′ is selected from —H, —OH, —OCH₃, —CH₃, —CH₂CH₂OCH₃, and

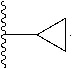 [SMILES: *C1CC1]

99. The compound of claim 96, wherein Y₂′ is selected from —H, alkyl, alkoxy, haloalkyl, and hydroxyalkyl.

100. The compound of claim 99, wherein Y₂′ is selected from —H, —OCH₃, —CH₃, —CH₂CH₃, —CH₂OH, —CH₂CH₂OH, —CH₂CH₂CH₂OH, —CH₂CH₂F, and —CH₂CH₂CH₂F.

101. The compound of claim 84, wherein Y₂ is —N(Y₂″)₂.

102. The compound of claim 101, wherein each Y₂″ is —CH₃.

103. The compound of claim 101, wherein both instances of Y₂″ taken together with the nitrogen atom to which they are bonded form a morpholinyl or azetidinyl.

104. The compound of claim 84, wherein Y₂′ is selected from cyanoalkyl, —O-alkoxyalkyl, —O-haloalkyl, and —O-hydroxyalkyl,

105. The compound of claim 84, wherein Y₂′ is selected from —CH₂CH₂CN, —OCH₂CH₂CH₂CN, —OCH₂CHF₂, —OCH₂CH₂CHF₂, —CH₂CH₂OH, —CH₂CH₂OCH₃, and —OCH₂CH₂CH₂OH.

106. The compound of claim 1 having the structure selected from:

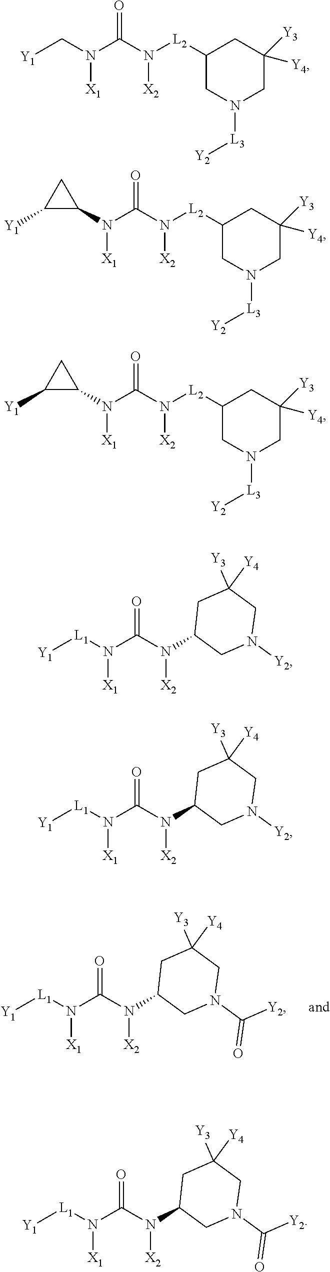

107. The compound of claim 31 having the structure:

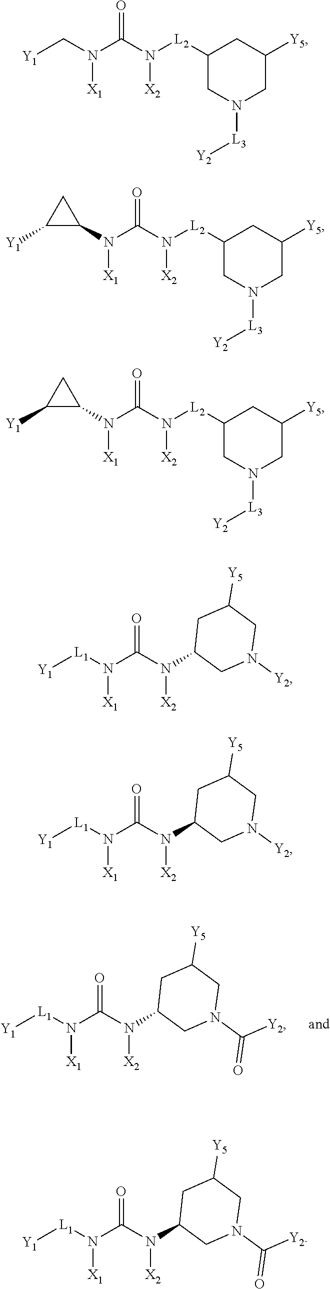

108. A compound or a pharmaceutically acceptable salt thereof having the structure of any one of the following compounds:

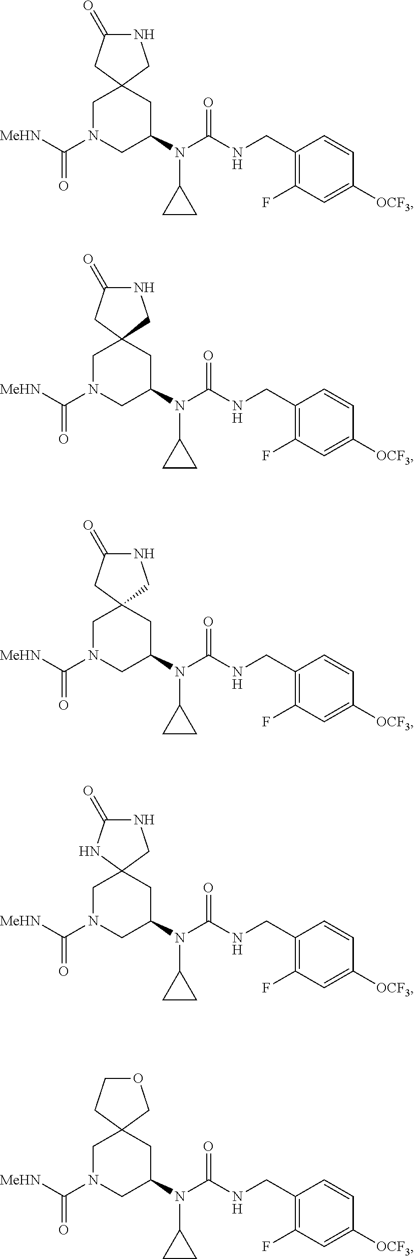 [SMILES: CNC(=O)N1C[C@H](N(C(=O)NCc2ccc(OC(F)(F)F)cc2F)C2CC2)CC2(CNC(=O)C2)C1 ; CNC(=O)N1C[C@H](N(C(=O)NCc2ccc(OC(F)(F)F)cc2F)C2CC2)C[C@]2(CNC(=O)C2)C1 ; CNC(=O)N1C[C@H](N(C(=O)NCc2ccc(OC(F)(F)F)cc2F)C2CC2)C[C@@]2(CNC(=O)C2)C1 ; CNC(=O)N1C[C@H](N(C(=O)NCc2ccc(OC(F)(F)F)cc2F)C2CC2)CC2(CNC(=O)N2)C1 ; CNC(=O)N1C[C@H](N(C(=O)NCc2ccc(OC(F)(F)F)cc2F)C2CC2)CC2(CCOC2)C1]

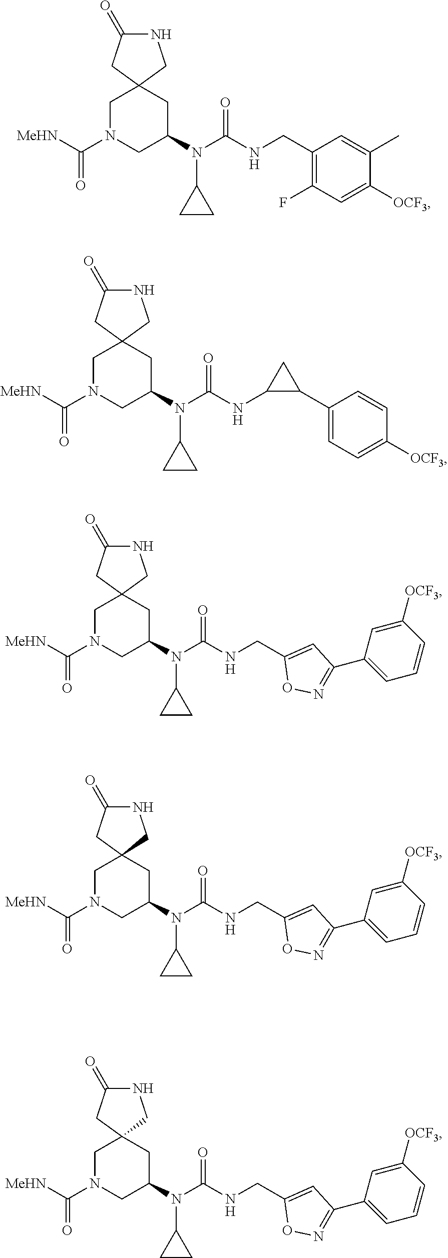 [SMILES: CNC(=O)N1C[C@H](N(C(=O)NCc2cc(C)c(OC(F)(F)F)cc2F)C2CC2)CC2(CNC(=O)C2)C1 ; CNC(=O)N1C[C@H](N(C(=O)NC2CC2c2ccc(OC(F)(F)F)cc2)C2CC2)CC2(CNC(=O)C2)C1 ; CNC(=O)N1C[C@H](N(C(=O)NCc2cc(-c3cccc(OC(F)(F)F)c3)no2)C2CC2)CC2(CNC(=O)C2)C1 ; CNC(=O)N1C[C@H](N(C(=O)NCc2cc(-c3cccc(OC(F)(F)F)c3)no2)C2CC2)C[C@]2(CNC(=O)C2)C1 ; CNC(=O)N1C[C@H](N(C(=O)NCc2cc(-c3cccc(OC(F)(F)F)c3)no2)C2CC2)C[C@@]2(CNC(=O)C2)C1]

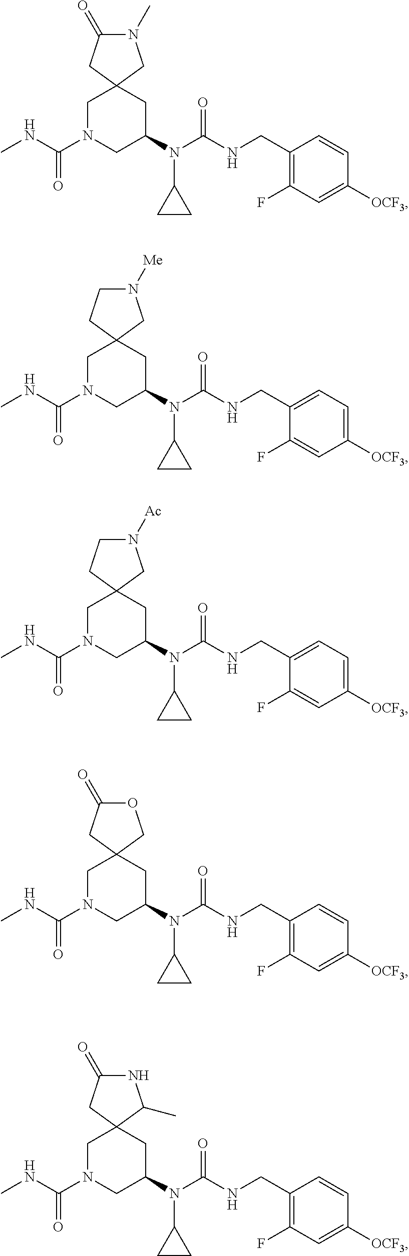

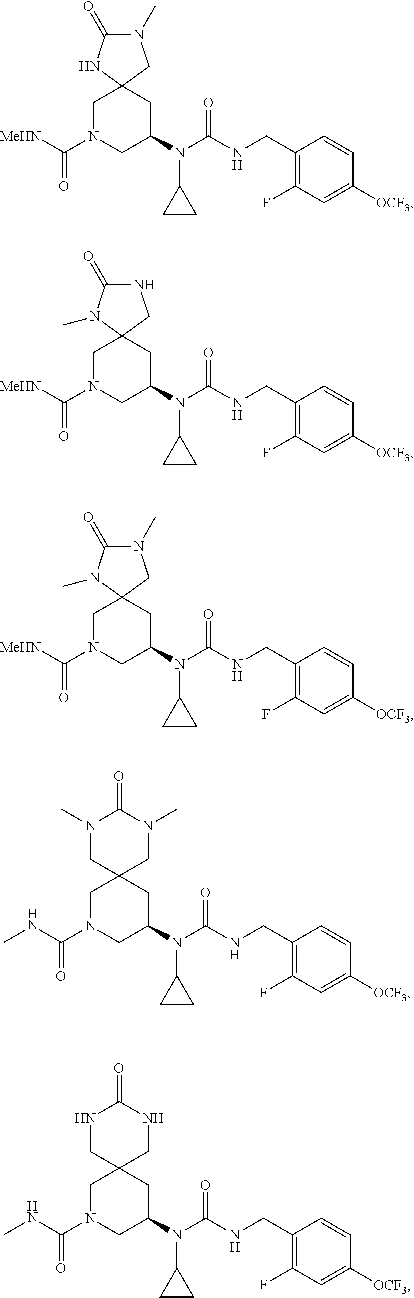 [SMILES: CNC(=O)N1C[C@H](N(C(=O)NCc2ccc(OC(F)(F)F)cc2F)C2CC2)CC2(CN(C)C(=O)N2)C1 ; CNC(=O)N1C[C@H](N(C(=O)NCc2ccc(OC(F)(F)F)cc2F)C2CC2)CC2(CNC(=O)N2C)C1 ; CNC(=O)N1C[C@H](N(C(=O)NCc2ccc(OC(F)(F)F)cc2F)C2CC2)CC2(CN(C)C(=O)N2C)C1 ; CNC(=O)N1C[C@H](N(C(=O)NCc2ccc(OC(F)(F)F)cc2F)C2CC2)CC2(CN(C)C(=O)N(C)C2)C1 ; CNC(=O)N1C[C@H](N(C(=O)NCc2ccc(OC(F)(F)F)cc2F)C2CC2)CC2(CNC(=O)NC2)C1]

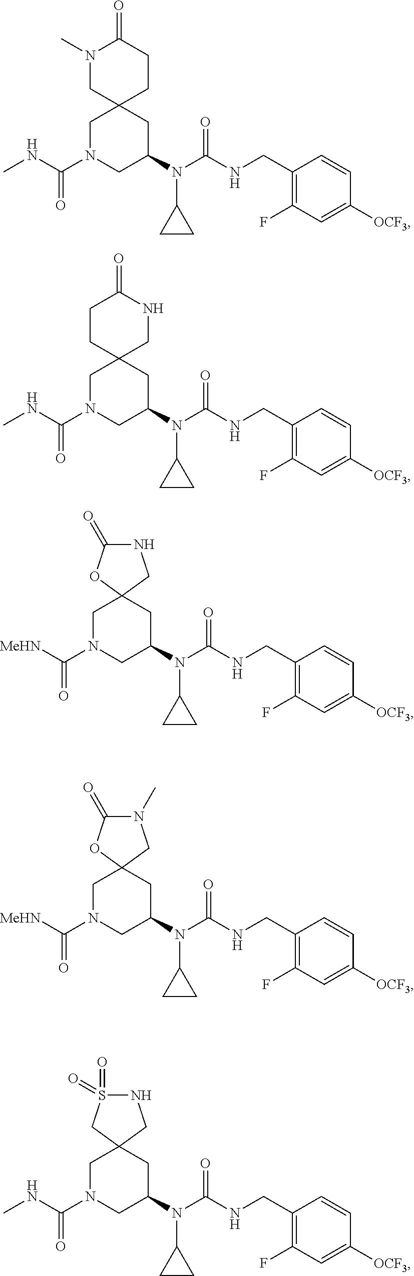 [SMILES: CNC(=O)N1C[C@H](N(C(=O)NCc2ccc(OC(F)(F)F)cc2F)C2CC2)CC2(CCC(=O)N(C)C2)C1 ; CNC(=O)N1C[C@H](N(C(=O)NCc2ccc(OC(F)(F)F)cc2F)C2CC2)CC2(CCC(=O)NC2)C1 ; CNC(=O)N1C[C@H](N(C(=O)NCc2ccc(OC(F)(F)F)cc2F)C2CC2)CC2(CNC(=O)O2)C1 ; CNC(=O)N1C[C@H](N(C(=O)NCc2ccc(OC(F)(F)F)cc2F)C2CC2)CC2(CN(C)C(=O)O2)C1 ; CNC(=O)N1C[C@H](N(C(=O)NCc2ccc(OC(F)(F)F)cc2F)C2CC2)CC2(CNS(=O)(=O)C2)C1]

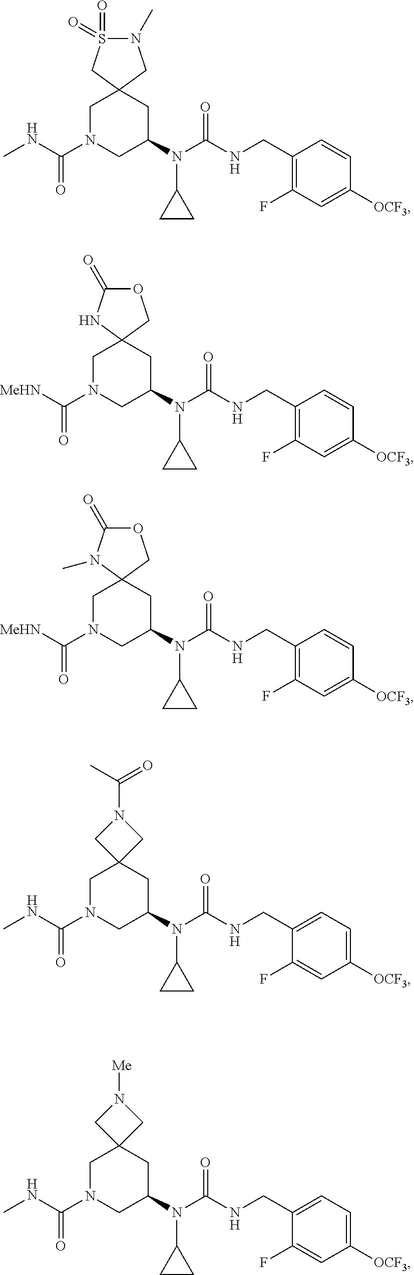 [SMILES: CNC(=O)N1C[C@H](N(C(=O)NCc2ccc(OC(F)(F)F)cc2F)C2CC2)CC2(C1)CN(C)S(=O)(=O)C2 ; CNC(=O)N1C[C@H](N(C(=O)NCc2ccc(OC(F)(F)F)cc2F)C2CC2)CC2(COC(=O)N2)C1 ; CNC(=O)N1C[C@H](N(C(=O)NCc2ccc(OC(F)(F)F)cc2F)C2CC2)CC2(COC(=O)N2C)C1 ; CNC(=O)N1C[C@H](N(C(=O)NCc2ccc(OC(F)(F)F)cc2F)C2CC2)CC2(CN(C(C)=O)C2)C1 ; CNC(=O)N1C[C@H](N(C(=O)NCc2ccc(OC(F)(F)F)cc2F)C2CC2)CC2(CN(C)C2)C1]

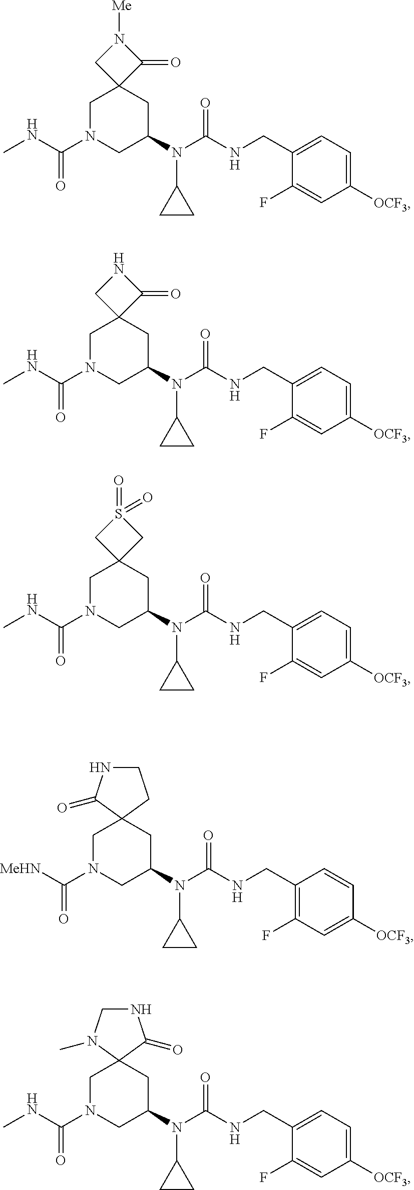 [SMILES: CNC(=O)N1C[C@H](N(C(=O)NCc2ccc(OC(F)(F)F)cc2F)C2CC2)CC2(CN(C)C2=O)C1 ; CNC(=O)N1C[C@H](N(C(=O)NCc2ccc(OC(F)(F)F)cc2F)C2CC2)CC2(CNC2=O)C1 ; CNC(=O)N1C[C@H](N(C(=O)NCc2ccc(OC(F)(F)F)cc2F)C2CC2)CC2(C1)CS(=O)(=O)C2 ; CNC(=O)N1C[C@H](N(C(=O)NCc2ccc(OC(F)(F)F)cc2F)C2CC2)CC2(CCNC2=O)C1 ; CNC(=O)N1C[C@H](N(C(=O)NCc2ccc(OC(F)(F)F)cc2F)C2CC2)CC2(C1)C(=O)NCN2C]

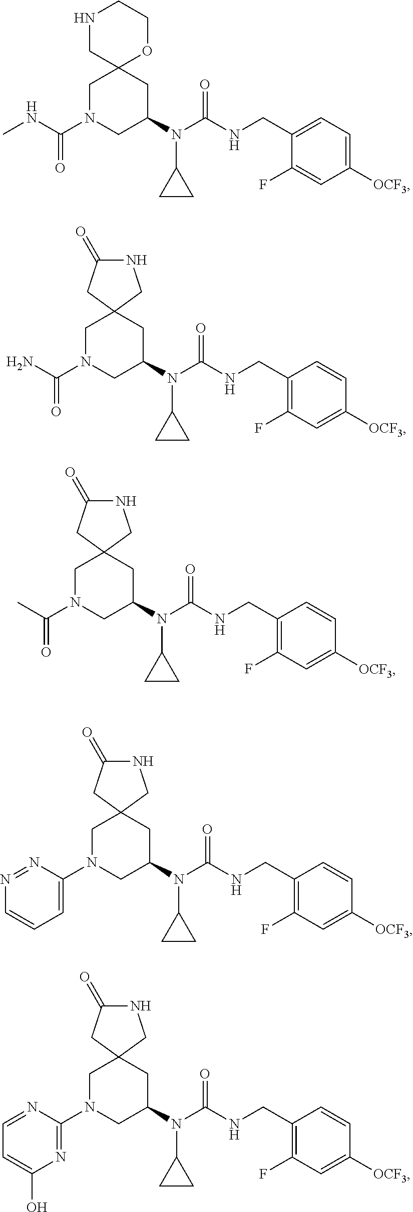 [SMILES: CNC(=O)N1C[C@H](N(C(=O)NCc2ccc(OC(F)(F)F)cc2F)C2CC2)CC2(CNCCO2)C1 ; NC(=O)N1C[C@H](N(C(=O)NCc2ccc(OC(F)(F)F)cc2F)C2CC2)CC2(CNC(=O)C2)C1 ; CC(=O)N1C[C@H](N(C(=O)NCc2ccc(OC(F)(F)F)cc2F)C2CC2)CC2(CNC(=O)C2)C1 ; O=C1CC2(CN1)C[C@@H](N(C(=O)NCc1ccc(OC(F)(F)F)cc1F)C1CC1)CN(c1cccnn1)C2 ; O=C1CC2(CN1)C[C@@H](N(C(=O)NCc1ccc(OC(F)(F)F)cc1F)C1CC1)CN(c1nccc(O)n1)C2]

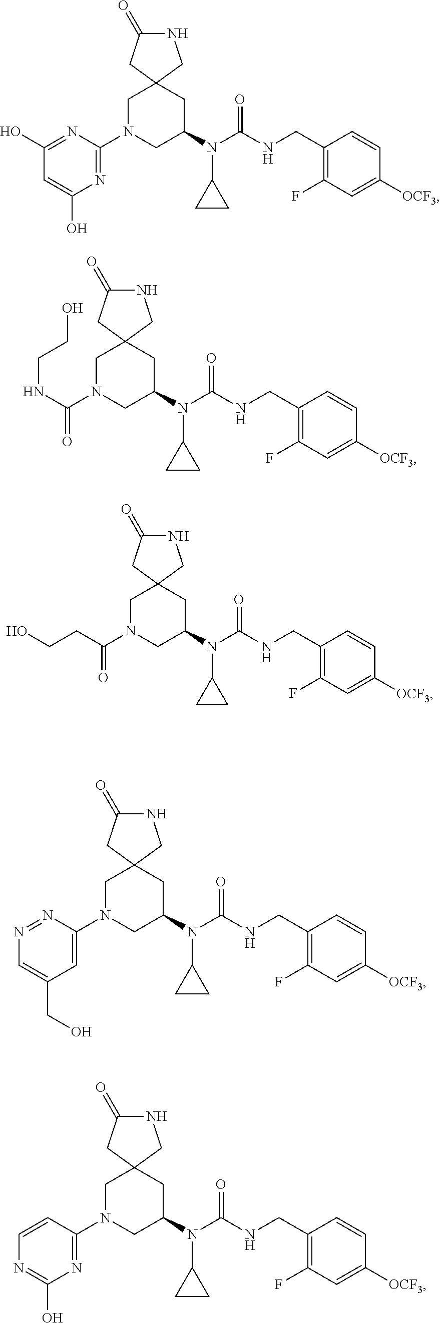

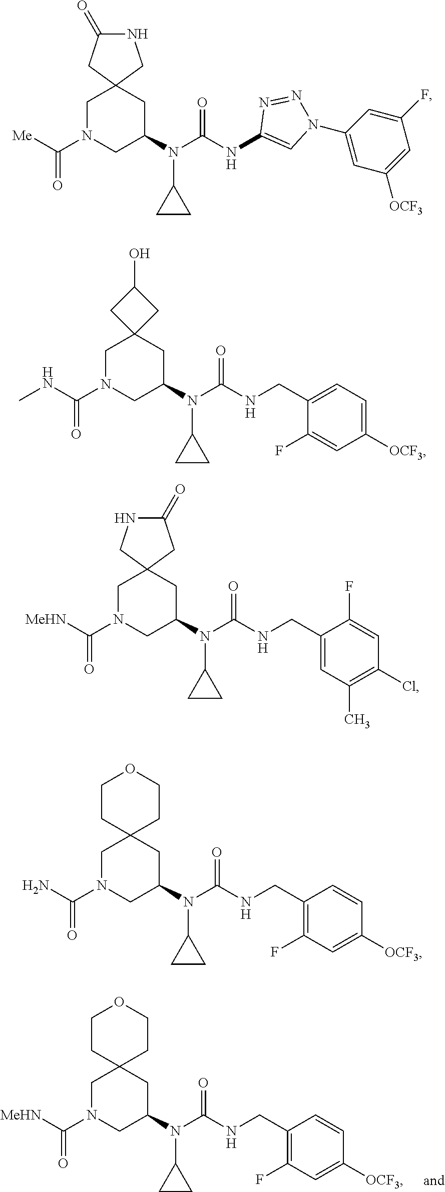

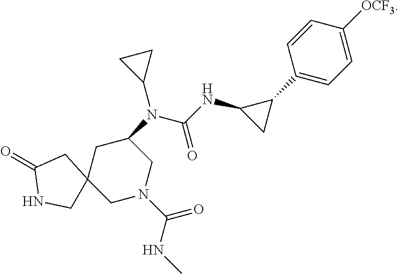 [SMILES: CNC(=O)N1C[C@H](N(C(=O)N[C@@H]2C[C@H]2c2ccc(OC(F)(F)F)cc2)C2CC2)CC2(CNC(=O)C2)C1]

109. A compound or a pharmaceutically acceptable salt thereof having the structure of any one of the following compounds:

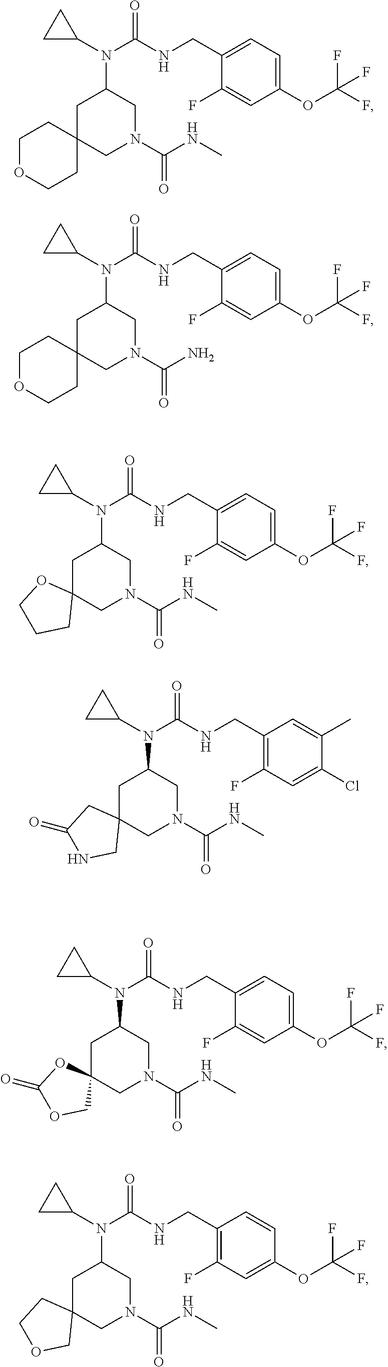

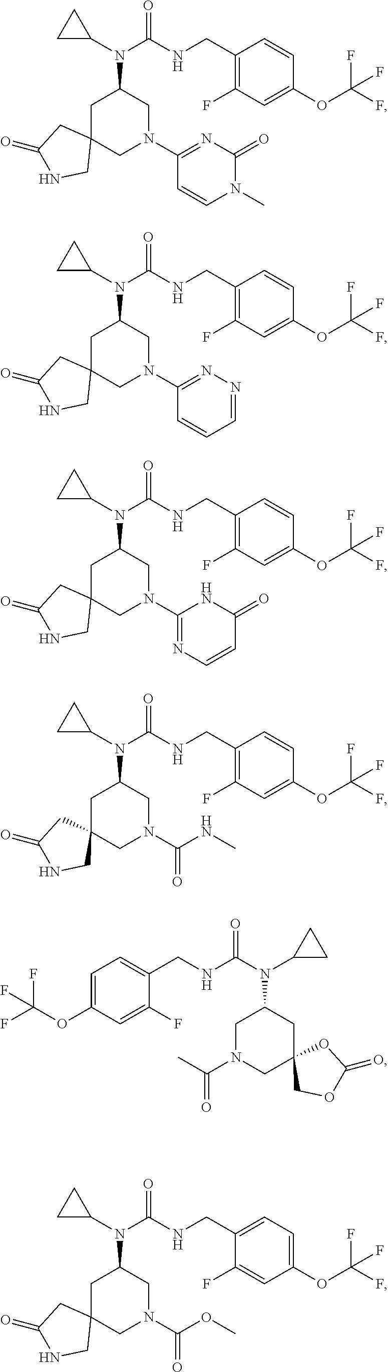 [SMILES: Cn1ccc(N2C[C@H](N(C(=O)NCc3ccc(OC(F)(F)F)cc3F)C3CC3)CC3(CNC(=O)C3)C2)nc1=O ; O=C1CC2(CN1)C[C@@H](N(C(=O)NCc1ccc(OC(F)(F)F)cc1F)C1CC1)CN(c1cccnn1)C2 ; O=C1CC2(CN1)C[C@@H](N(C(=O)NCc1ccc(OC(F)(F)F)cc1F)C1CC1)CN(c1nccc(=O)[nH]1)C2 ; CNC(=O)N1C[C@H](N(C(=O)NCc2ccc(OC(F)(F)F)cc2F)C2CC2)C[C@]2(CNC(=O)C2)C1 ; CC(=O)N1C[C@H](N(C(=O)NCc2ccc(OC(F)(F)F)cc2F)C2CC2)C[C@@]2(COC(=O)O2)C1 ; COC(=O)N1C[C@H](N(C(=O)NCc2ccc(OC(F)(F)F)cc2F)C2CC2)CC2(CNC(=O)C2)C1]

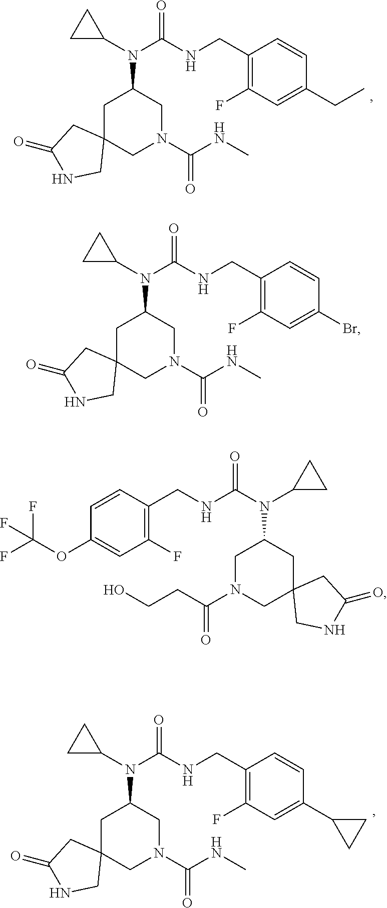

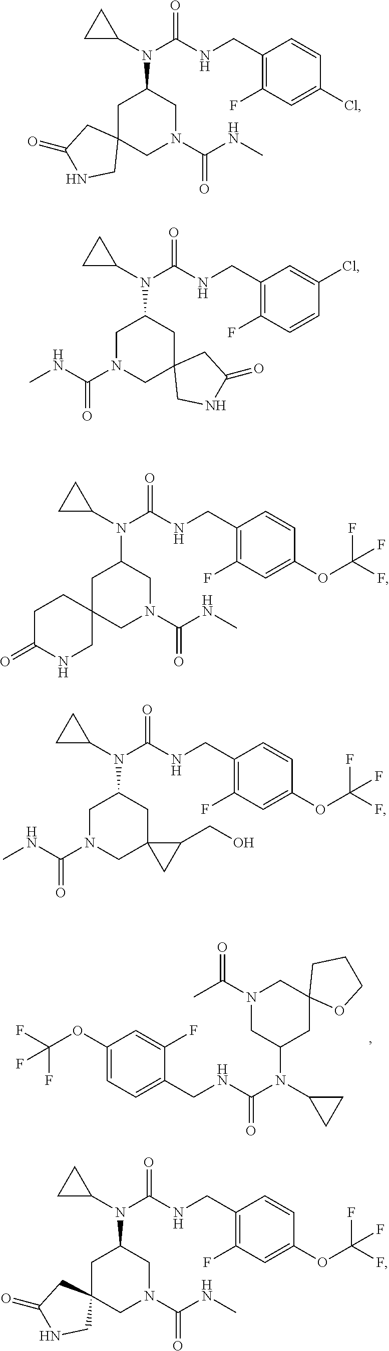 [SMILES: CNC(=O)N1C[C@H](N(C(=O)NCc2ccc(Cl)cc2F)C2CC2)CC2(CNC(=O)C2)C1 ; CNC(=O)N1C[C@H](N(C(=O)NCc2cc(Cl)ccc2F)C2CC2)CC2(CNC(=O)C2)C1 ; CNC(=O)N1CC(N(C(=O)NCc2ccc(OC(F)(F)F)cc2F)C2CC2)CC2(CCC(=O)NC2)C1 ; CNC(=O)N1C[C@H](N(C(=O)NCc2ccc(OC(F)(F)F)cc2F)C2CC2)CC2(CC2CO)C1 ; CC(=O)N1CC(N(C(=O)NCc2ccc(OC(F)(F)F)cc2F)C2CC2)CC2(CCCO2)C1 ; CNC(=O)N1C[C@H](N(C(=O)NCc2ccc(OC(F)(F)F)cc2F)C2CC2)C[C@@]2(CNC(=O)C2)C1]

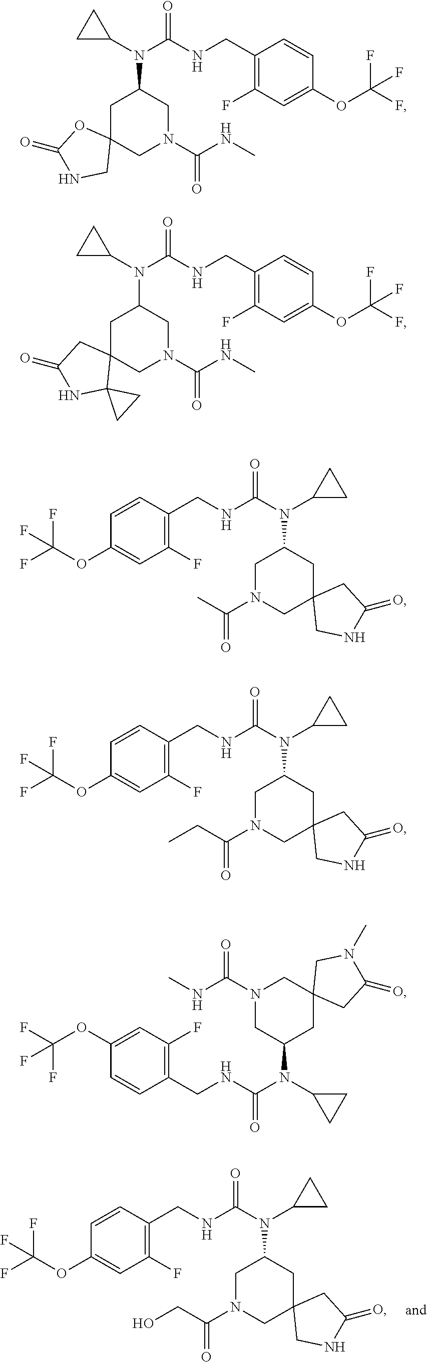

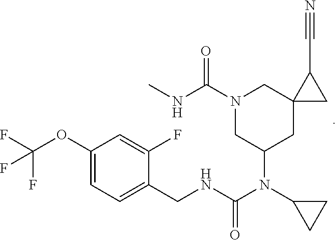 [SMILES: CNC(=O)N1CC(N(C(=O)NCc2ccc(OC(F)(F)F)cc2F)C2CC2)CC2(CC2C#N)C1]

110. A compound or a pharmaceutically acceptable salt thereof having the structure of any one of the following compounds:

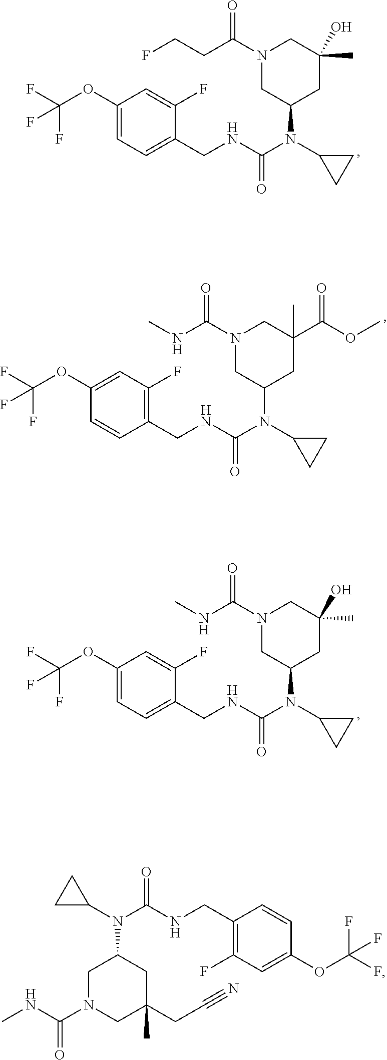

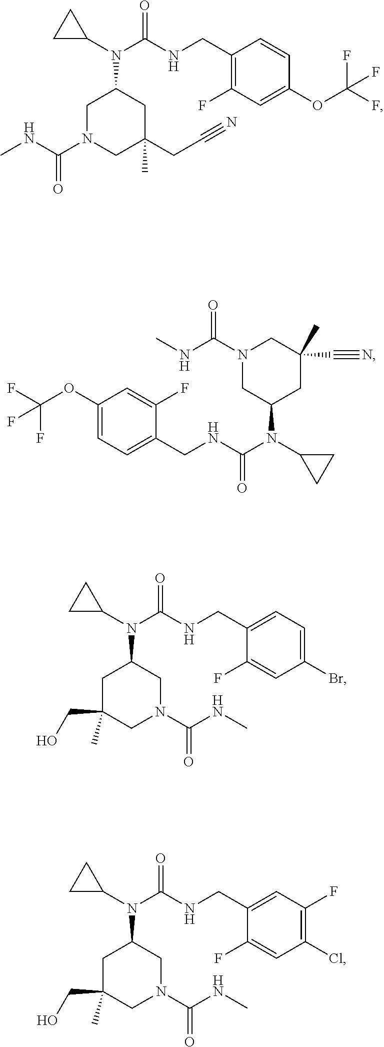 [SMILES: CNC(=O)N1C[C@H](N(C(=O)NCc2ccc(OC(F)(F)F)cc2F)C2CC2)C[C@@](C)(CC#N)C1 ; CNC(=O)N1C[C@H](N(C(=O)NCc2ccc(OC(F)(F)F)cc2F)C2CC2)C[C@@](C)(C#N)C1 ; CNC(=O)N1C[C@H](N(C(=O)NCc2ccc(Br)cc2F)C2CC2)C[C@](C)(CO)C1 ; CNC(=O)N1C[C@H](N(C(=O)NCc2cc(F)c(Cl)cc2F)C2CC2)C[C@](C)(CO)C1]

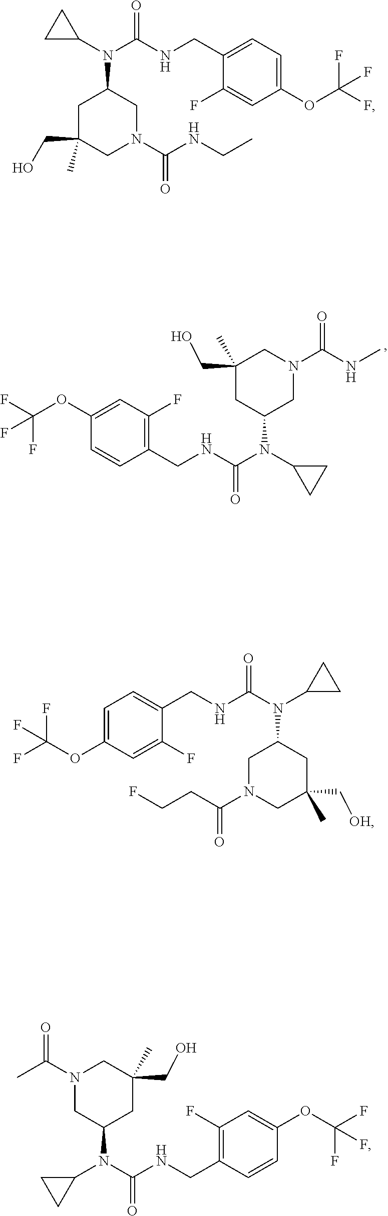

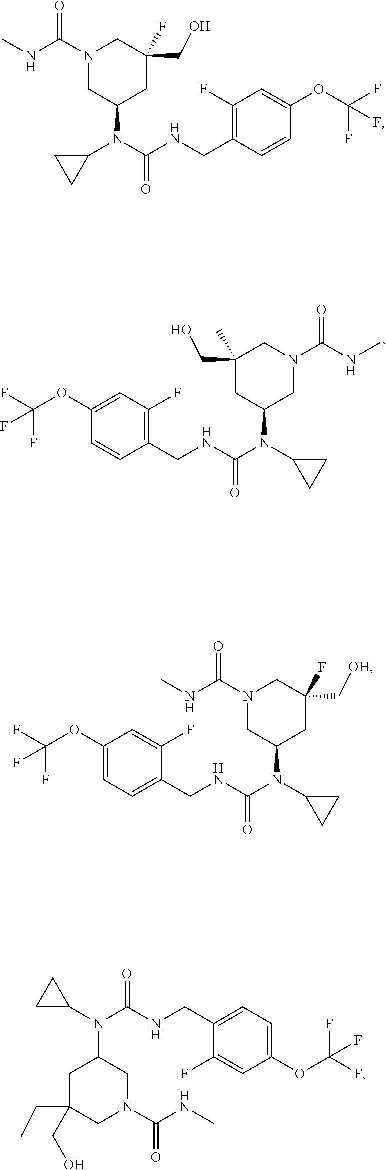

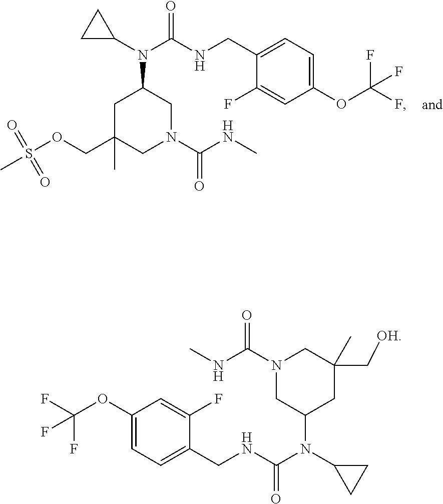 [SMILES: CNC(=O)N1C[C@H](N(C(=O)NCc2ccc(OC(F)(F)F)cc2F)C2CC2)CC(C)(COS(C)(=O)=O)C1 ; "and" ; CNC(=O)N1CC(N(C(=O)NCc2ccc(OC(F)(F)F)cc2F)C2CC2)CC(C)(CO)C1]

111. A compound or a pharmaceutically acceptable salt thereof having the structure of any one of the following compounds:

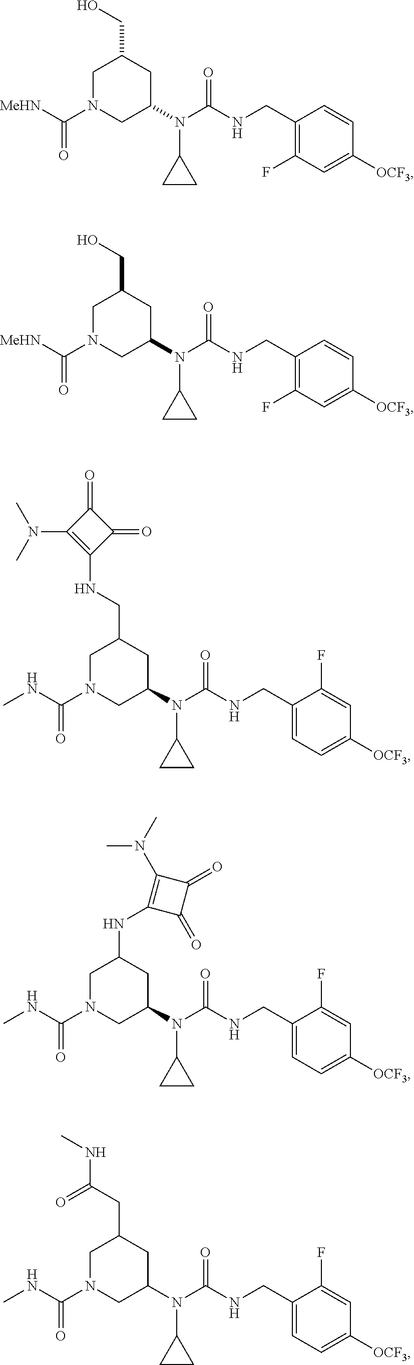

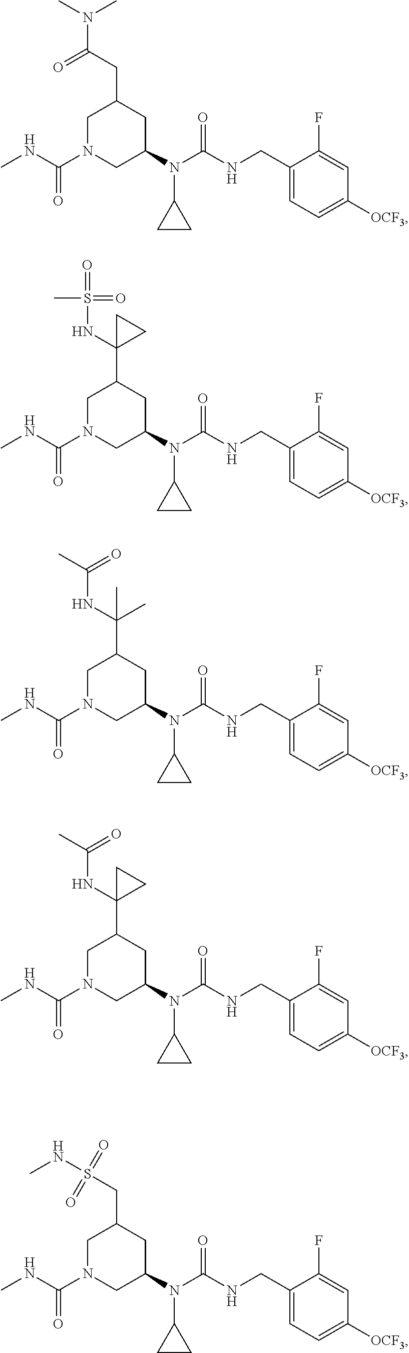 [SMILES: CNC(=O)N1CC(CC(=O)N(C)C)C[C@@H](N(C(=O)NCc2ccc(OC(F)(F)F)cc2F)C2CC2)C1 ; CNC(=O)N1CC(C2(NS(C)(=O)=O)CC2)C[C@@H](N(C(=O)NCc2ccc(OC(F)(F)F)cc2F)C2CC2)C1 ; CNC(=O)N1CC(C(C)(C)NC(C)=O)C[C@@H](N(C(=O)NCc2ccc(OC(F)(F)F)cc2F)C2CC2)C1 ; CNC(=O)N1CC(C2(NC(C)=O)CC2)C[C@@H](N(C(=O)NCc2ccc(OC(F)(F)F)cc2F)C2CC2)C1 ; CNC(=O)N1CC(CS(=O)(=O)NC)C[C@@H](N(C(=O)NCc2ccc(OC(F)(F)F)cc2F)C2CC2)C1]

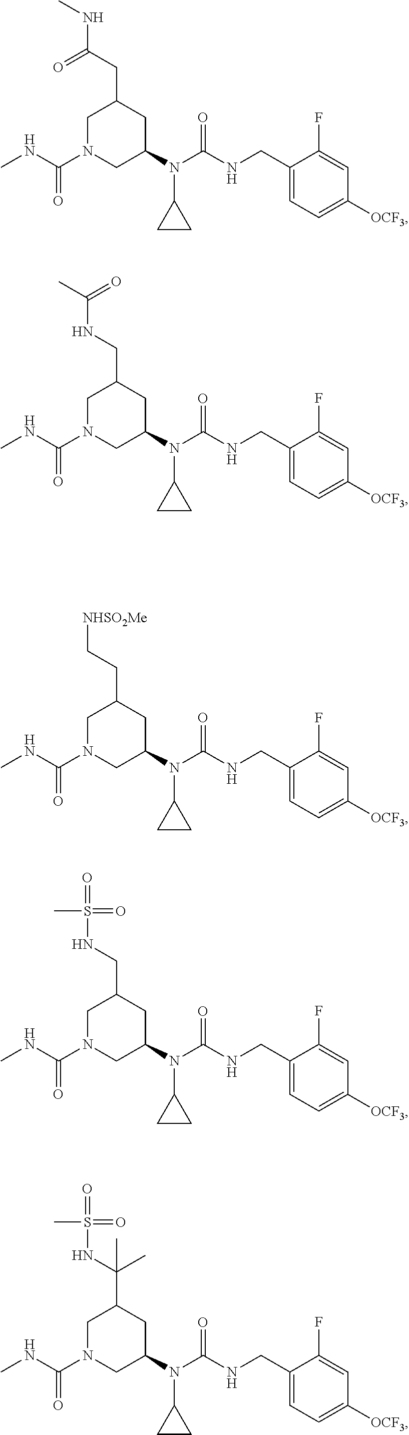

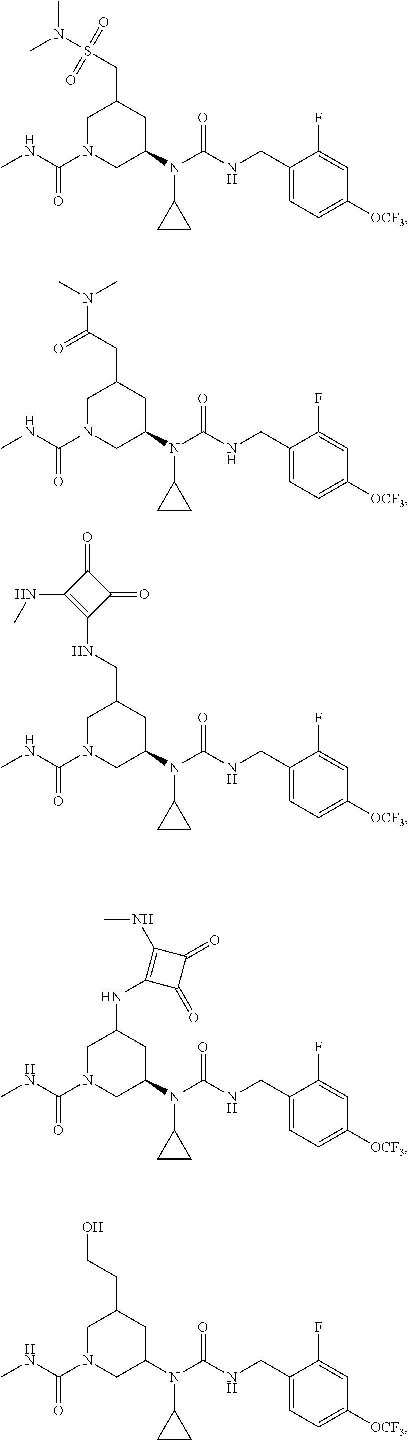 [SMILES: CNC(=O)N1CC(CS(=O)(=O)N(C)C)C[C@@H](N(C(=O)NCc2ccc(OC(F)(F)F)cc2F)C2CC2)C1 ; CNC(=O)N1CC(CC(=O)N(C)C)C[C@@H](N(C(=O)NCc2ccc(OC(F)(F)F)cc2F)C2CC2)C1 ; CNC(=O)N1CC(CNc2c(NC)c(=O)c2=O)C[C@@H](N(C(=O)NCc2ccc(OC(F)(F)F)cc2F)C2CC2)C1 ; CNC(=O)N1CC(Nc2c(NC)c(=O)c2=O)C[C@@H](N(C(=O)NCc2ccc(OC(F)(F)F)cc2F)C2CC2)C1 ; CNC(=O)N1CC(CCO)CC(N(C(=O)NCc2ccc(OC(F)(F)F)cc2F)C2CC2)C1]

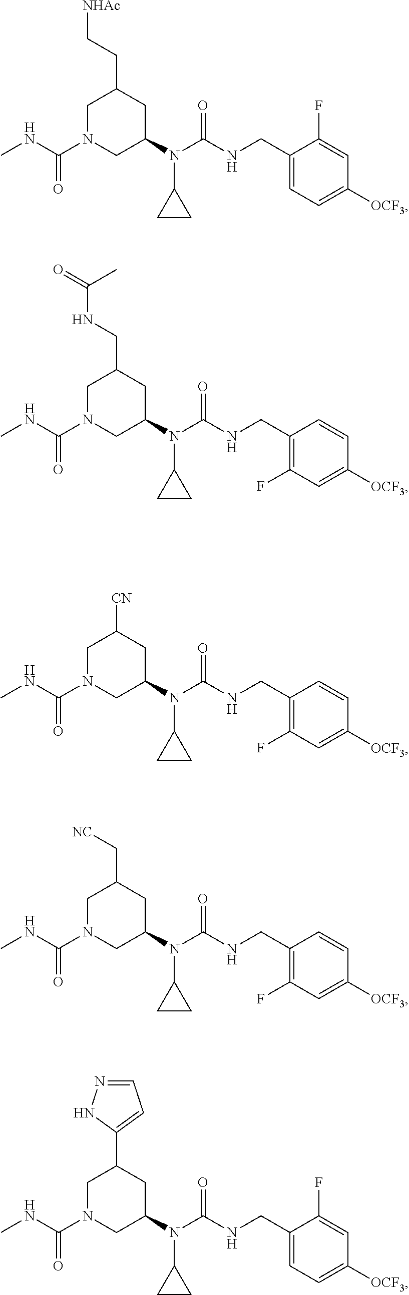 [SMILES: CNC(=O)N1CC(CCNC(C)=O)C[C@@H](N(C(=O)NCc2ccc(OC(F)(F)F)cc2F)C2CC2)C1 ; CNC(=O)N1CC(CNC(C)=O)C[C@@H](N(C(=O)NCc2ccc(OC(F)(F)F)cc2F)C2CC2)C1 ; CNC(=O)N1CC(C#N)C[C@@H](N(C(=O)NCc2ccc(OC(F)(F)F)cc2F)C2CC2)C1 ; CNC(=O)N1CC(CC#N)C[C@@H](N(C(=O)NCc2ccc(OC(F)(F)F)cc2F)C2CC2)C1 ; CNC(=O)N1CC(c2ccn[nH]2)C[C@@H](N(C(=O)NCc2ccc(OC(F)(F)F)cc2F)C2CC2)C1]

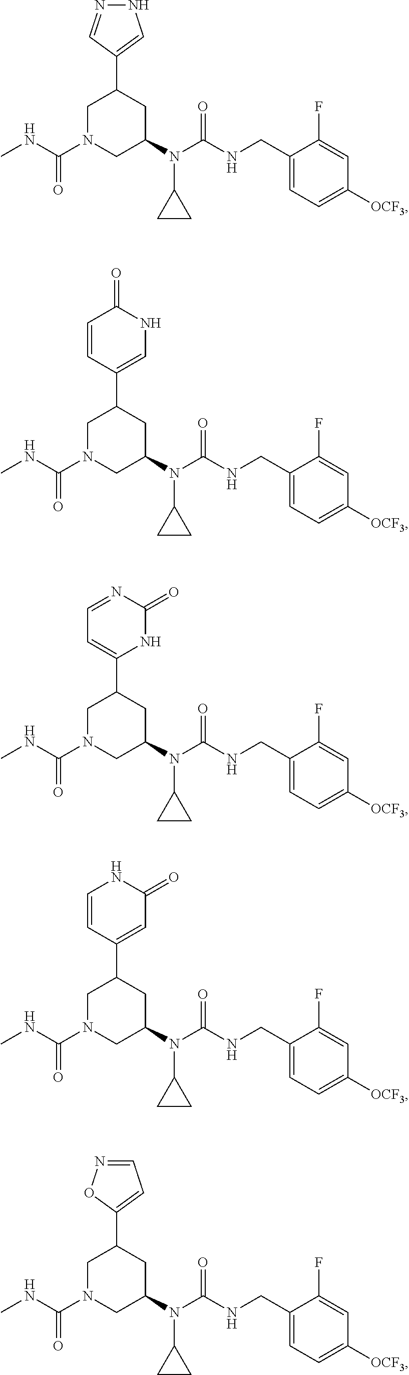 [SMILES: CNC(=O)N1CC(c2cn[nH]c2)C[C@@H](N(C(=O)NCc2ccc(OC(F)(F)F)cc2F)C2CC2)C1 ; CNC(=O)N1CC(c2ccc(=O)[nH]c2)C[C@@H](N(C(=O)NCc2ccc(OC(F)(F)F)cc2F)C2CC2)C1 ; CNC(=O)N1CC(c2ccnc(=O)[nH]2)C[C@@H](N(C(=O)NCc2ccc(OC(F)(F)F)cc2F)C2CC2)C1 ; CNC(=O)N1CC(c2cc[nH]c(=O)c2)C[C@@H](N(C(=O)NCc2ccc(OC(F)(F)F)cc2F)C2CC2)C1 ; CNC(=O)N1CC(c2ccno2)C[C@@H](N(C(=O)NCc2ccc(OC(F)(F)F)cc2F)C2CC2)C1]

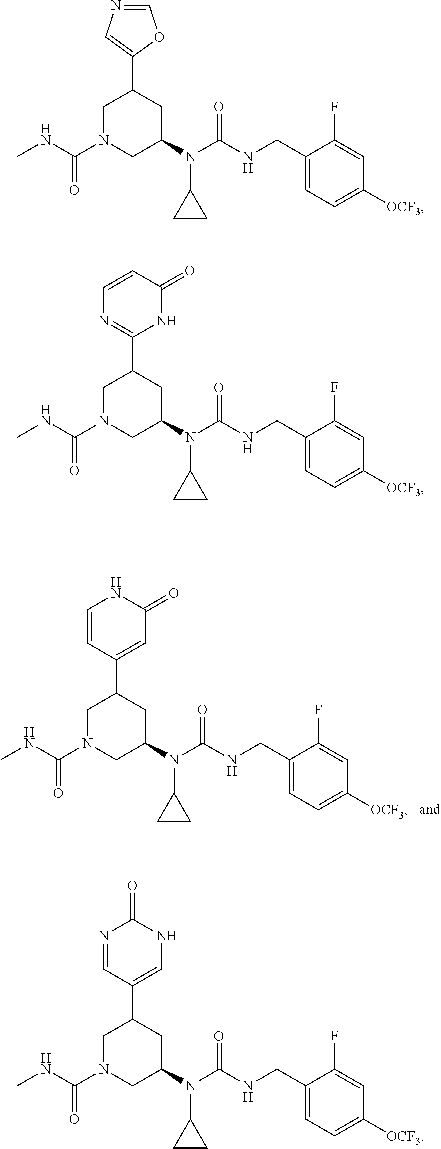 [SMILES: CNC(=O)N1CC(c2cnco2)C[C@@H](N(C(=O)NCc2ccc(OC(F)(F)F)cc2F)C2CC2)C1 ; CNC(=O)N1CC(c2nccc(=O)[nH]2)C[C@@H](N(C(=O)NCc2ccc(OC(F)(F)F)cc2F)C2CC2)C1 ; CNC(=O)N1CC(c2cc[nH]c(=O)c2)C[C@@H](N(C(=O)NCc2ccc(OC(F)(F)F)cc2F)C2CC2)C1 ; CNC(=O)N1CC(c2cnc(=O)[nH]c2)C[C@@H](N(C(=O)NCc2ccc(OC(F)(F)F)cc2F)C2CC2)C1]

112. A compound or a pharmaceutically acceptable salt thereof having the structure of any one of the following compounds:

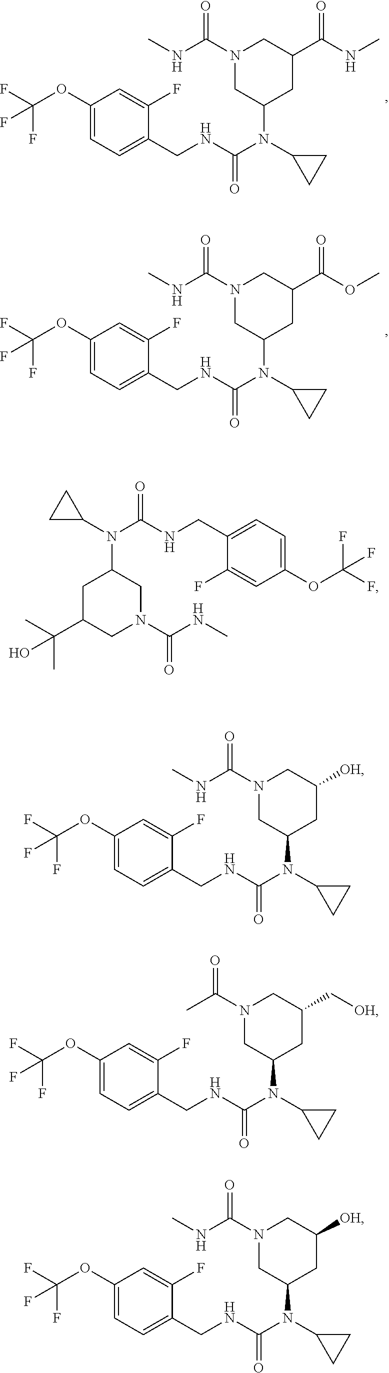

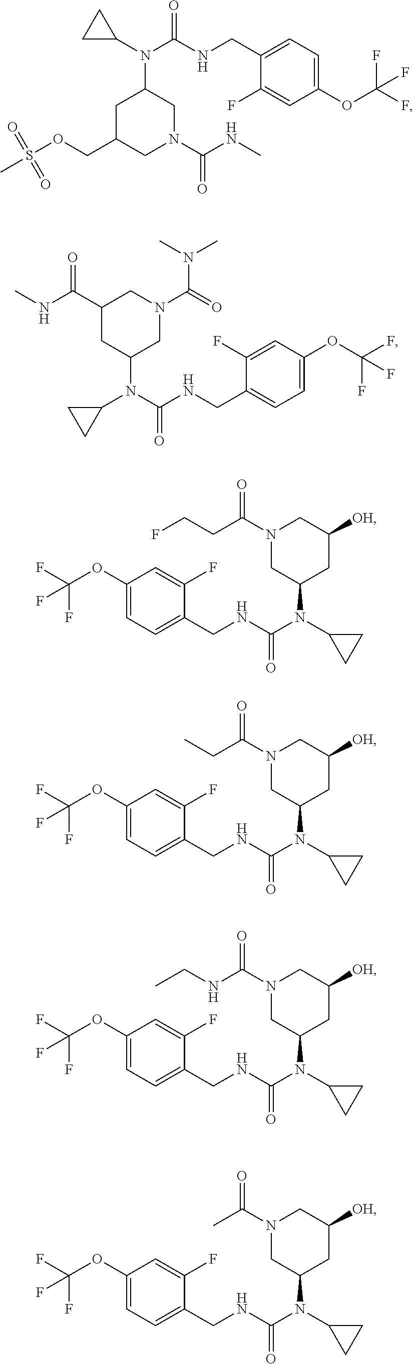 [SMILES: CNC(=O)N1CC(COS(C)(=O)=O)CC(N(C(=O)NCc2ccc(OC(F)(F)F)cc2F)C2CC2)C1 ; CNC(=O)C1CC(N(C(=O)NCc2ccc(OC(F)(F)F)cc2F)C2CC2)CN(C(=O)N(C)C)C1 ; O=C(CCF)N1C[C@@H](O)C[C@@H](N(C(=O)NCc2ccc(OC(F)(F)F)cc2F)C2CC2)C1 ; CCC(=O)N1C[C@@H](O)C[C@@H](N(C(=O)NCc2ccc(OC(F)(F)F)cc2F)C2CC2)C1 ; CCNC(=O)N1C[C@@H](O)C[C@@H](N(C(=O)NCc2ccc(OC(F)(F)F)cc2F)C2CC2)C1 ; CC(=O)N1C[C@@H](O)C[C@@H](N(C(=O)NCc2ccc(OC(F)(F)F)cc2F)C2CC2)C1]

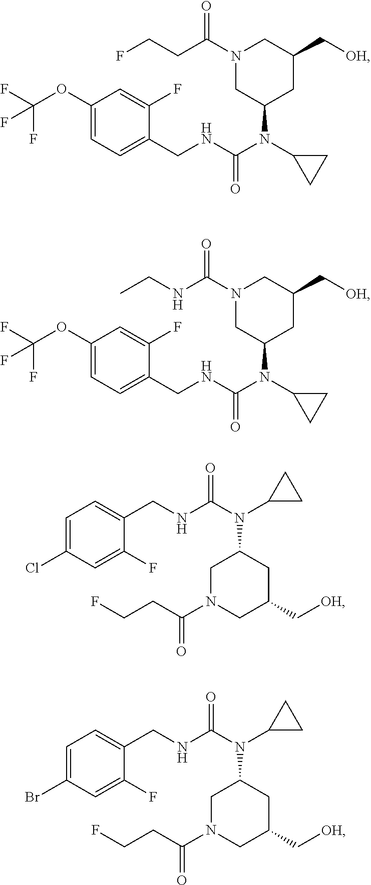 [SMILES: O=C(CCF)N1C[C@@H](CO)C[C@@H](N(C(=O)NCc2ccc(OC(F)(F)F)cc2F)C2CC2)C1 ; CCNC(=O)N1C[C@@H](CO)C[C@@H](N(C(=O)NCc2ccc(OC(F)(F)F)cc2F)C2CC2)C1 ; O=C(CCF)N1C[C@@H](CO)C[C@@H](N(C(=O)NCc2ccc(Cl)cc2F)C2CC2)C1 ; O=C(CCF)N1C[C@@H](CO)C[C@@H](N(C(=O)NCc2ccc(Br)cc2F)C2CC2)C1]

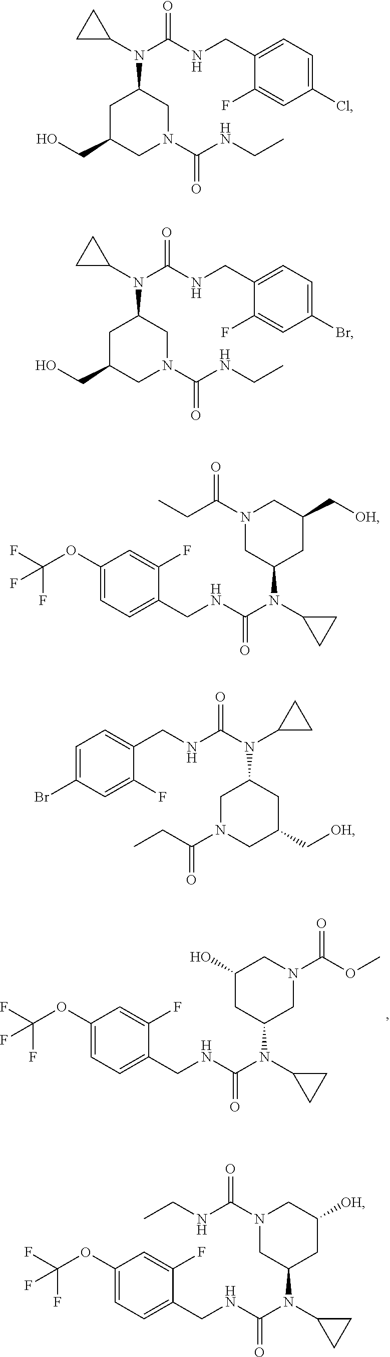 [SMILES: CCNC(=O)N1C[C@@H](CO)C[C@@H](N(C(=O)NCc2ccc(Cl)cc2F)C2CC2)C1 ; CCNC(=O)N1C[C@@H](CO)C[C@@H](N(C(=O)NCc2ccc(Br)cc2F)C2CC2)C1 ; CCC(=O)N1C[C@@H](CO)C[C@@H](N(C(=O)NCc2ccc(OC(F)(F)F)cc2F)C2CC2)C1 ; CCC(=O)N1C[C@@H](CO)C[C@@H](N(C(=O)NCc2ccc(Br)cc2F)C2CC2)C1 ; COC(=O)N1C[C@@H](O)C[C@@H](N(C(=O)NCc2ccc(OC(F)(F)F)cc2F)C2CC2)C1 ; CCNC(=O)N1C[C@H](O)C[C@@H](N(C(=O)NCc2ccc(OC(F)(F)F)cc2F)C2CC2)C1]

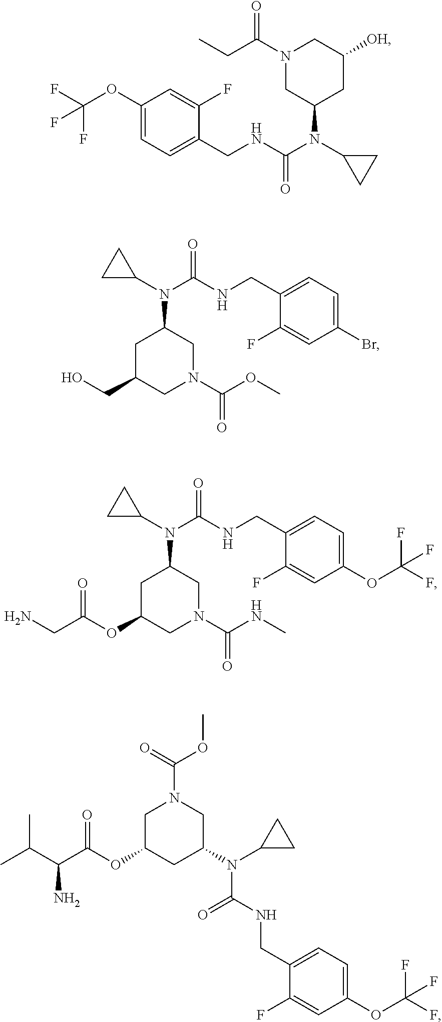 [SMILES: CCC(=O)N1C[C@H](O)C[C@@H](N(C(=O)NCc2ccc(OC(F)(F)F)cc2F)C2CC2)C1 ; COC(=O)N1C[C@@H](CO)C[C@@H](N(C(=O)NCc2ccc(Br)cc2F)C2CC2)C1 ; CNC(=O)N1C[C@@H](OC(=O)CN)C[C@@H](N(C(=O)NCc2ccc(OC(F)(F)F)cc2F)C2CC2)C1 ; COC(=O)N1C[C@@H](OC(=O)[C@@H](N)C(C)C)C[C@@H](N(C(=O)NCc2ccc(OC(F)(F)F)cc2F)C2CC2)C1]

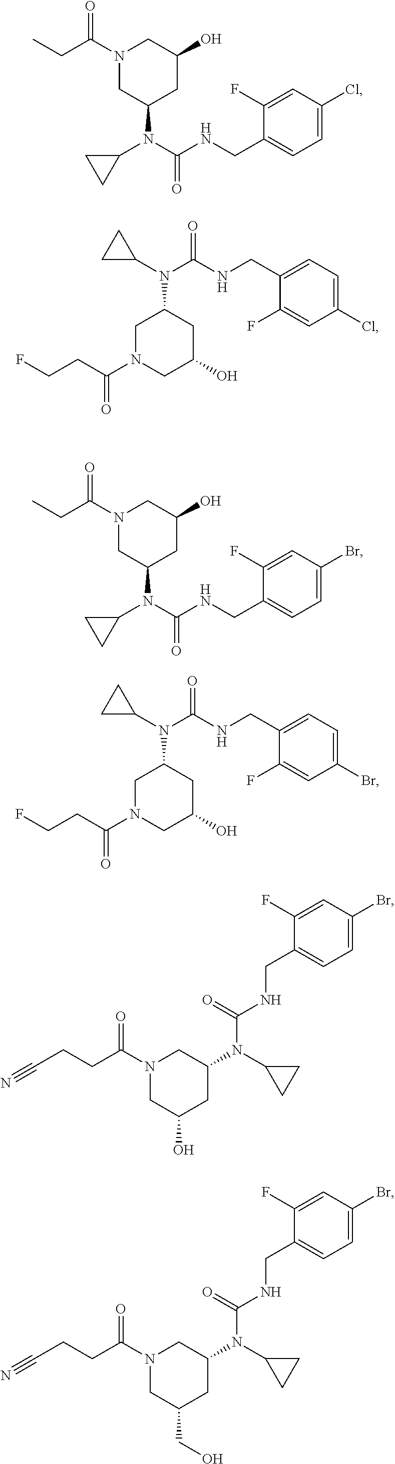

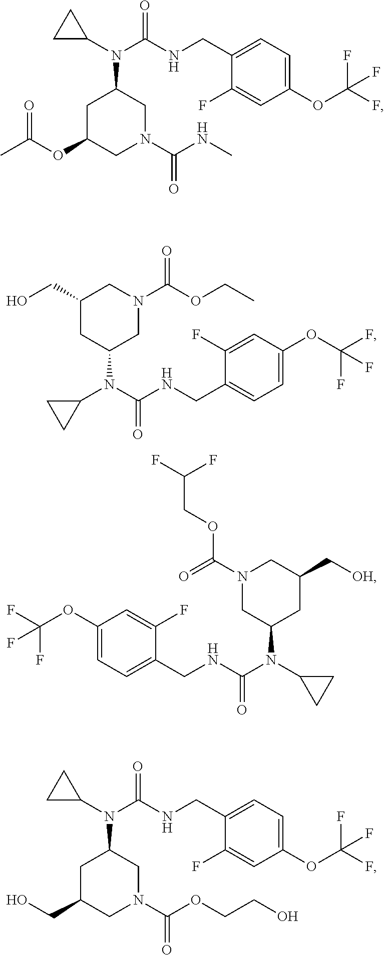

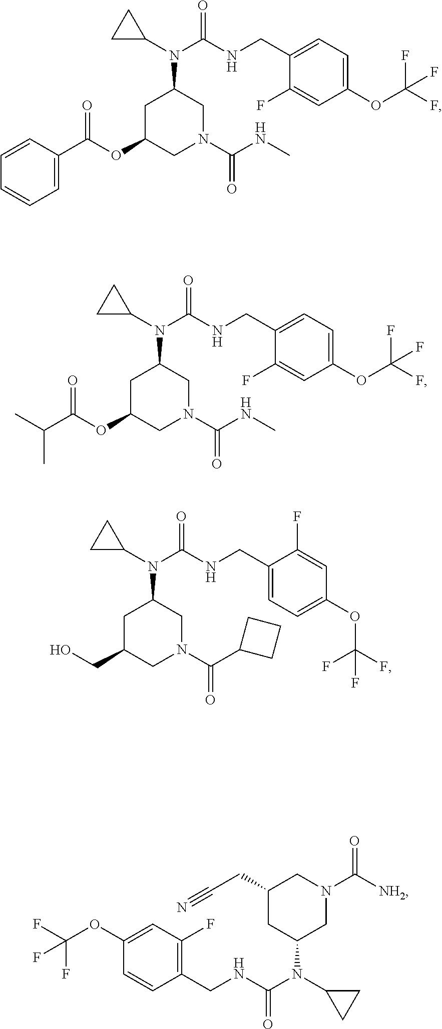 [SMILES: CNC(=O)N1C[C@@H](OC(=O)c2ccccc2)C[C@@H](N(C(=O)NCc2ccc(OC(F)(F)F)cc2F)C2CC2)C1 ; CNC(=O)N1C[C@@H](OC(=O)C(C)C)C[C@@H](N(C(=O)NCc2ccc(OC(F)(F)F)cc2F)C2CC2)C1 ; O=C(C1CCC1)N1C[C@@H](CO)C[C@@H](N(C(=O)NCc2ccc(OC(F)(F)F)cc2F)C2CC2)C1 ; N#CC[C@H]1C[C@@H](N(C(=O)NCc2ccc(OC(F)(F)F)cc2F)C2CC2)CN(C(N)=O)C1]

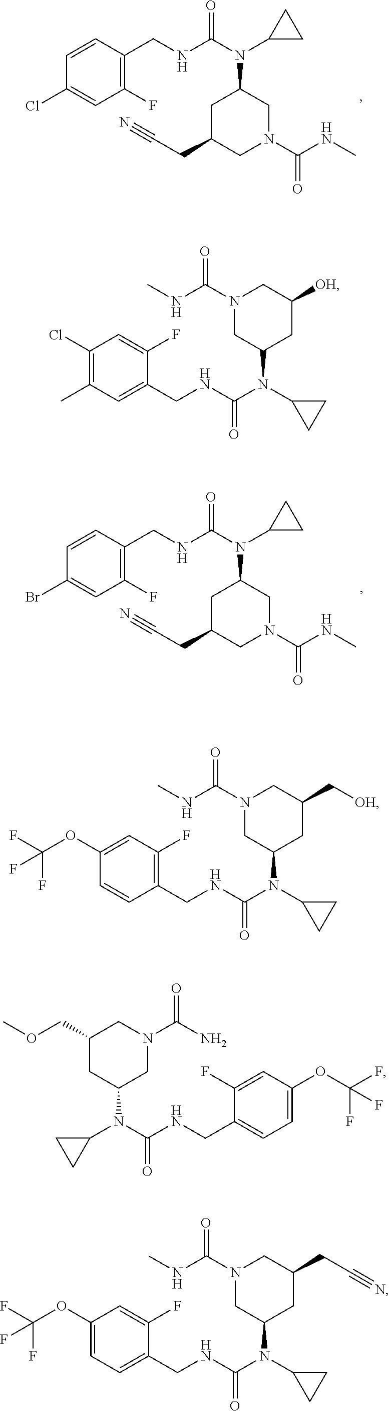

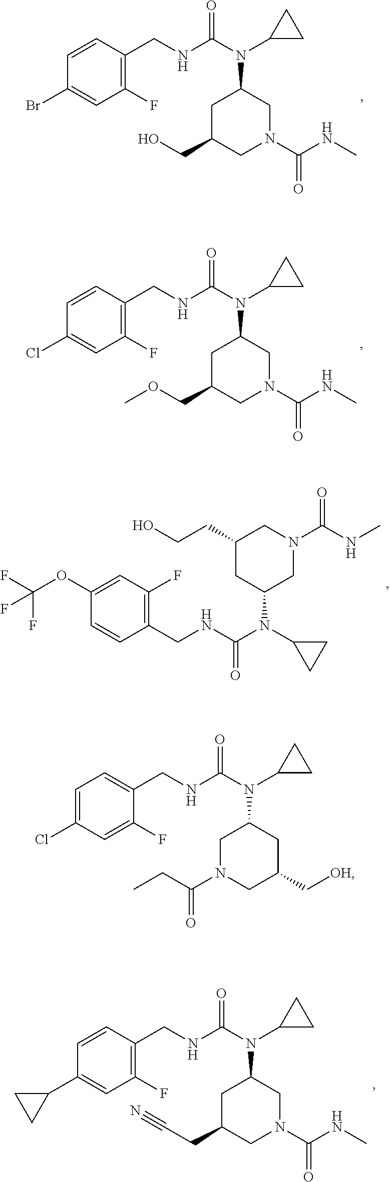 [SMILES: CNC(=O)N1C[C@@H](CO)C[C@@H](N(C(=O)NCc2ccc(Br)cc2F)C2CC2)C1 ; CNC(=O)N1C[C@@H](COC)C[C@@H](N(C(=O)NCc2ccc(Cl)cc2F)C2CC2)C1 ; CNC(=O)N1C[C@@H](CCO)C[C@@H](N(C(=O)NCc2ccc(OC(F)(F)F)cc2F)C2CC2)C1 ; CCC(=O)N1C[C@@H](CO)C[C@@H](N(C(=O)NCc2ccc(Cl)cc2F)C2CC2)C1 ; CNC(=O)N1C[C@@H](CC#N)C[C@@H](N(C(=O)NCc2ccc(C3CC3)cc2F)C2CC2)C1]

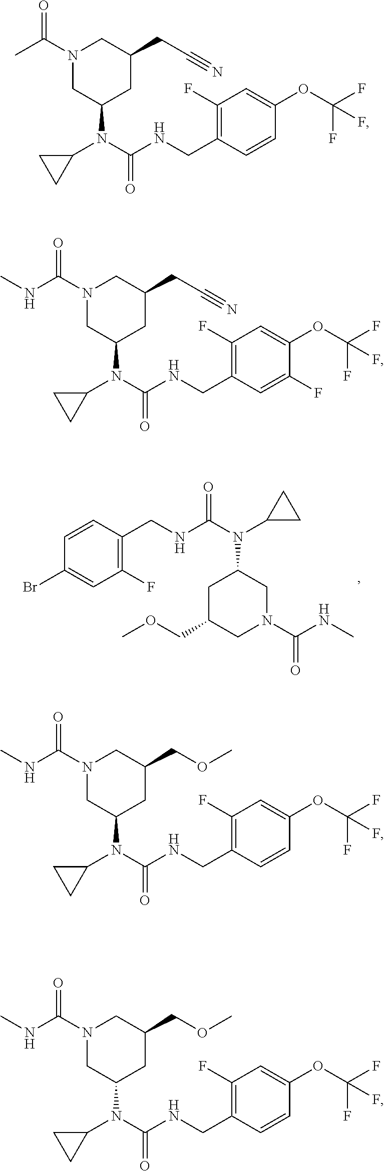

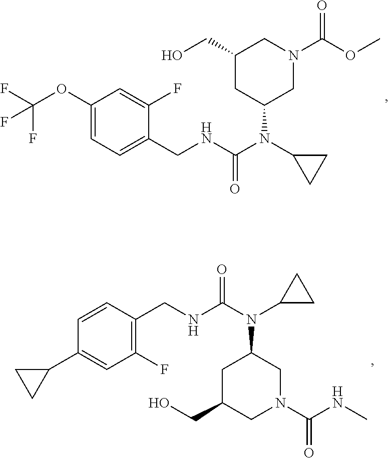 [SMILES: COC(=O)N1C[C@@H](CO)C[C@@H](N(C(=O)NCc2ccc(OC(F)(F)F)cc2F)C2CC2)C1 ; CNC(=O)N1C[C@@H](CO)C[C@@H](N(C(=O)NCc2ccc(C3CC3)cc2F)C2CC2)C1]

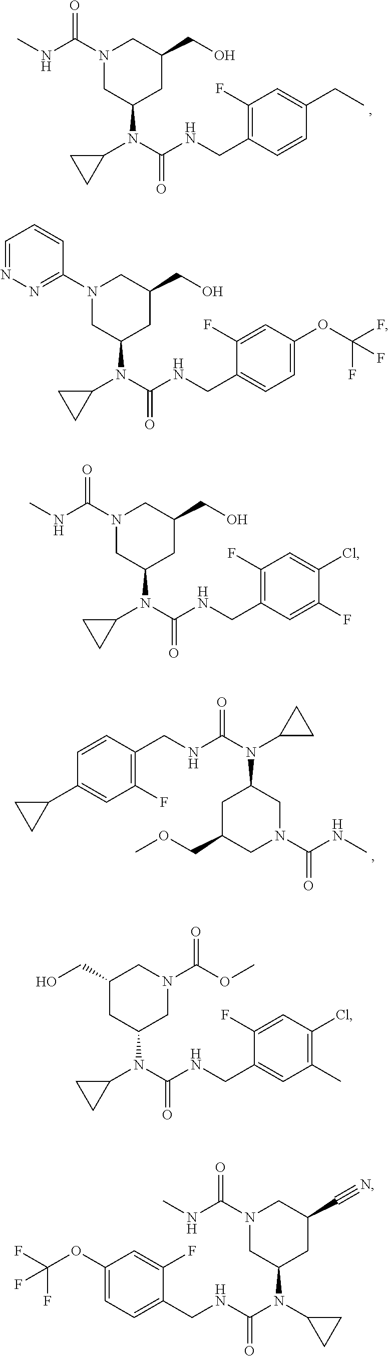

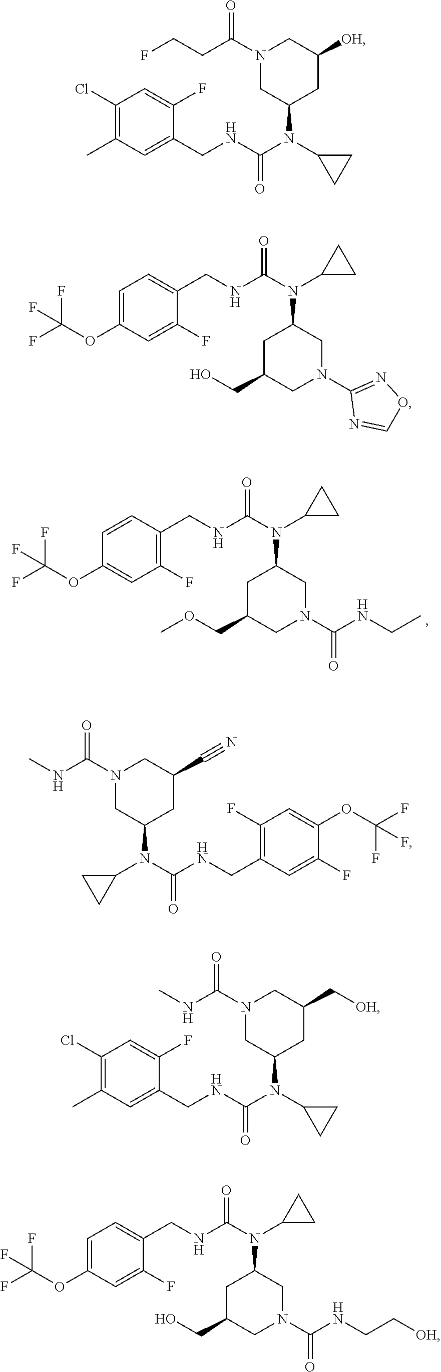

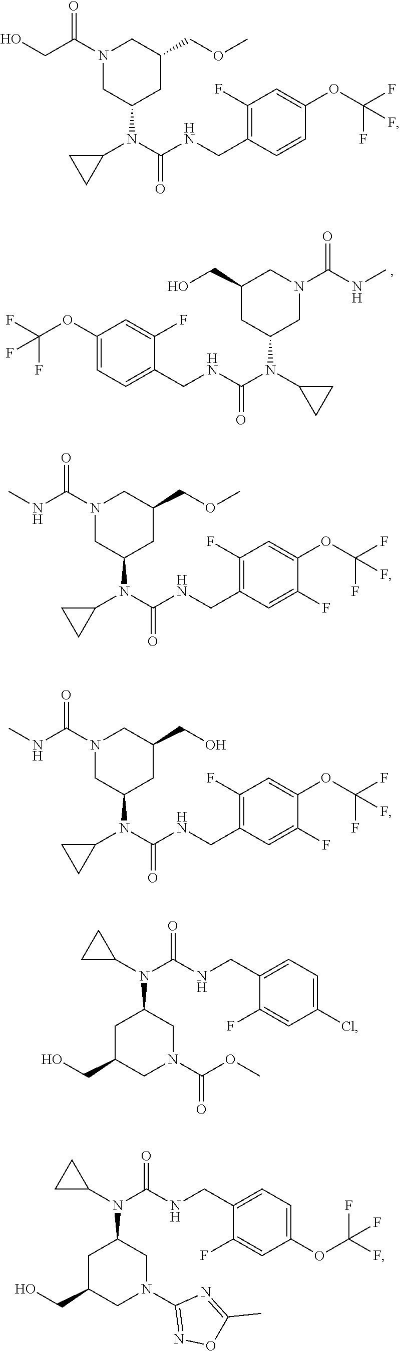

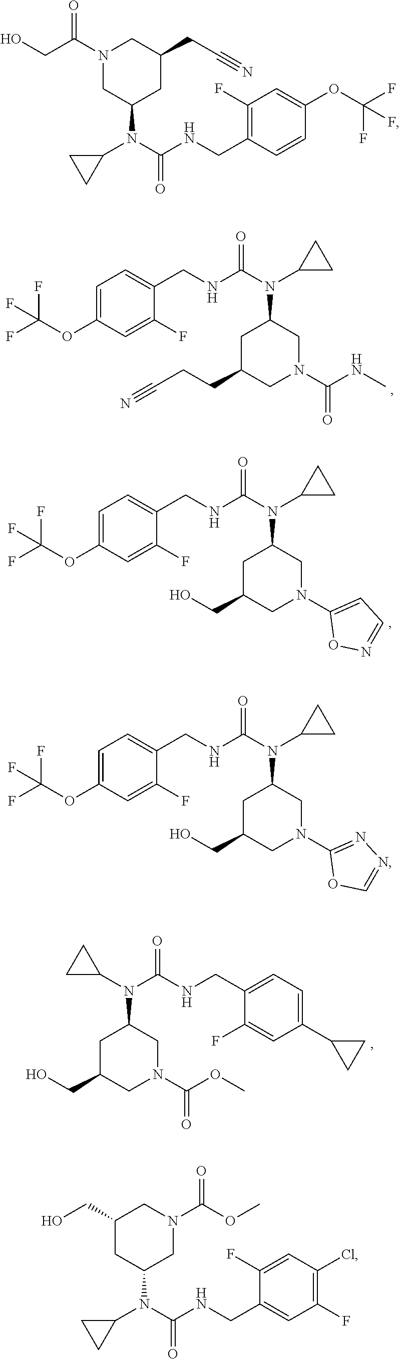 [SMILES: N#CC[C@H]1C[C@@H](N(C(=O)NCc2ccc(OC(F)(F)F)cc2F)C2CC2)CN(C(=O)CO)C1 ; CNC(=O)N1C[C@@H](CCC#N)C[C@@H](N(C(=O)NCc2ccc(OC(F)(F)F)cc2F)C2CC2)C1 ; O=C(NCc1ccc(OC(F)(F)F)cc1F)N(C1CC1)[C@@H]1C[C@H](CO)CN(c2ccno2)C1 ; O=C(NCc1ccc(OC(F)(F)F)cc1F)N(C1CC1)[C@@H]1C[C@H](CO)CN(c2nnco2)C1 ; COC(=O)N1C[C@@H](CO)C[C@@H](N(C(=O)NCc2ccc(C3CC3)cc2F)C2CC2)C1 ; COC(=O)N1C[C@@H](CO)C[C@@H](N(C(=O)NCc2cc(F)c(Cl)cc2F)C2CC2)C1]

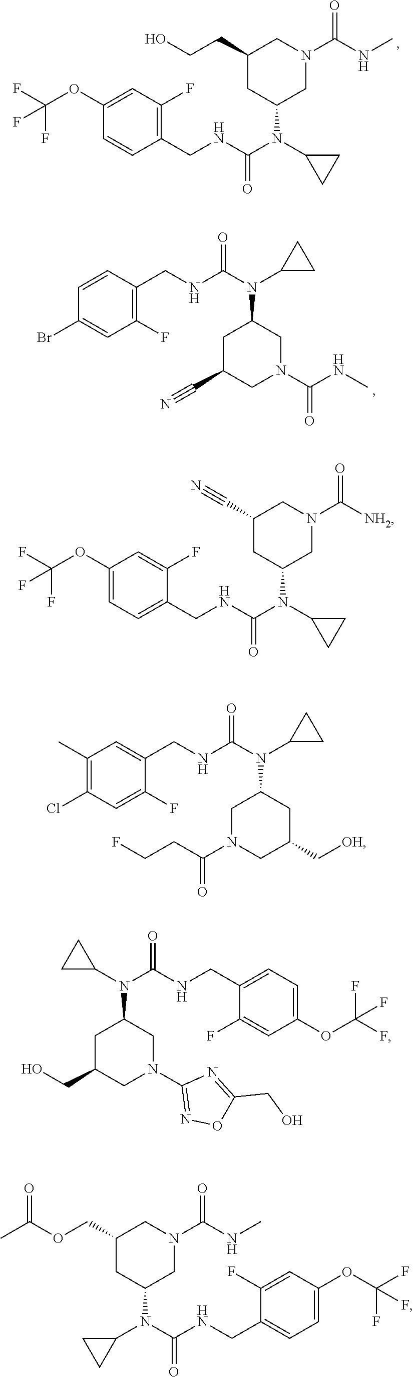

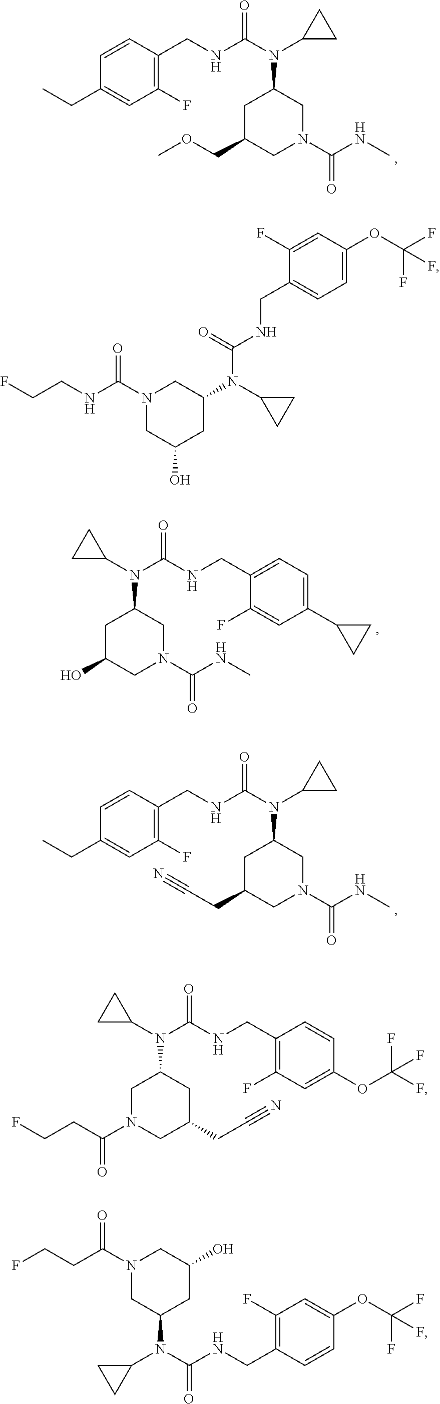

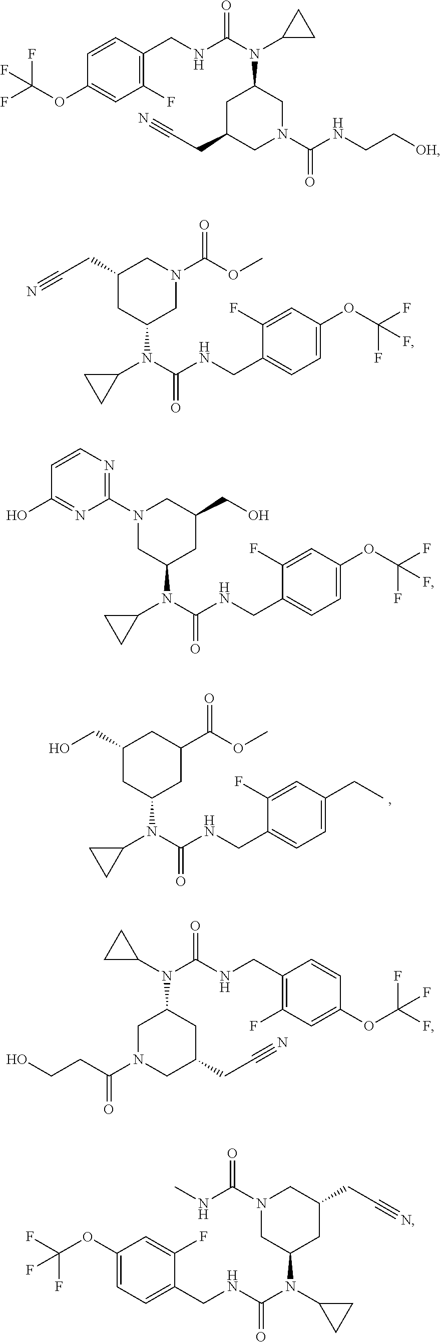 [SMILES: N#CC[C@H]1C[C@@H](N(C(=O)NCc2ccc(OC(F)(F)F)cc2F)C2CC2)CN(C(=O)NCCO)C1 ; COC(=O)N1C[C@@H](CC#N)C[C@@H](N(C(=O)NCc2ccc(OC(F)(F)F)cc2F)C2CC2)C1 ; O=C(NCc1ccc(OC(F)(F)F)cc1F)N(C1CC1)[C@@H]1C[C@H](CO)CN(c2nccc(O)n2)C1 ; CCc1ccc(CNC(=O)N(C2CC2)[C@H]2CC(C(=O)OC)C[C@@H](CO)C2)c(F)c1 ; N#CC[C@H]1C[C@@H](N(C(=O)NCc2ccc(OC(F)(F)F)cc2F)C2CC2)CN(C(=O)CCO)C1 ; CNC(=O)N1C[C@H](CC#N)C[C@@H](N(C(=O)NCc2ccc(OC(F)(F)F)cc2F)C2CC2)C1]

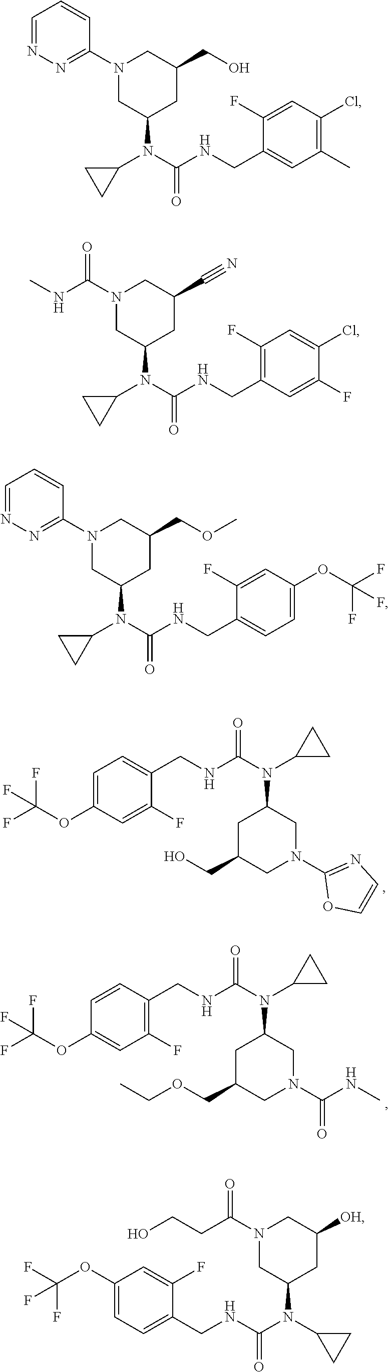

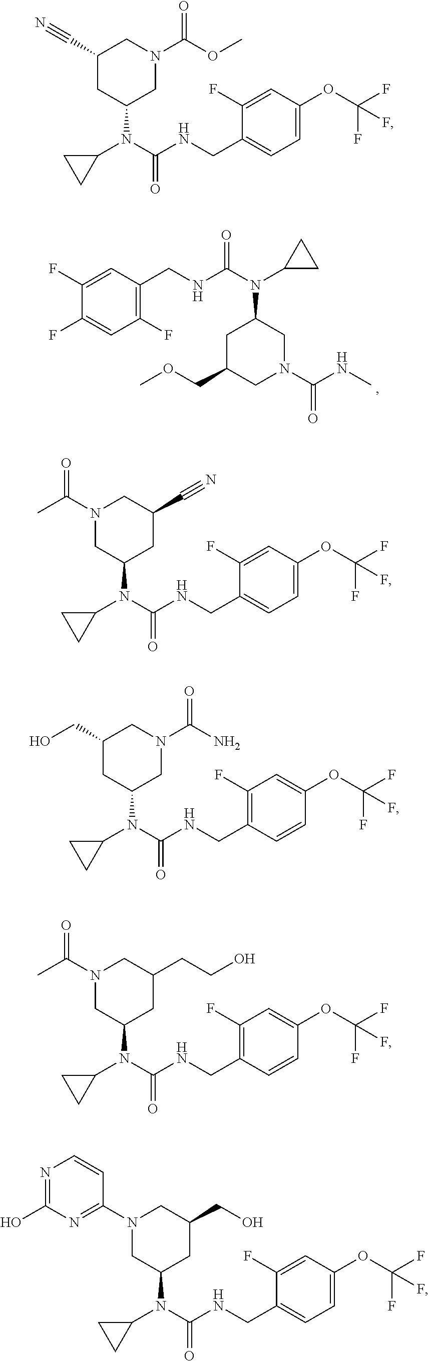

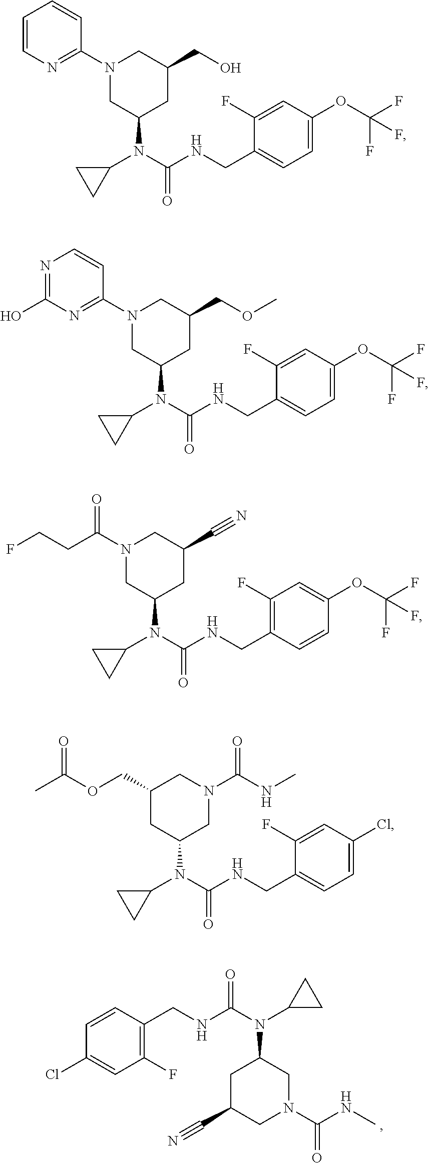 [SMILES: O=C(NCc1ccc(OC(F)(F)F)cc1F)N(C1CC1)[C@@H]1C[C@H](CO)CN(c2ccccn2)C1 ; COC[C@H]1C[C@@H](N(C(=O)NCc2ccc(OC(F)(F)F)cc2F)C2CC2)CN(c2ccnc(O)n2)C1 ; N#C[C@H]1C[C@@H](N(C(=O)NCc2ccc(OC(F)(F)F)cc2F)C2CC2)CN(C(=O)CCF)C1 ; CNC(=O)N1C[C@@H](COC(C)=O)C[C@@H](N(C(=O)NCc2ccc(Cl)cc2F)C2CC2)C1 ; CNC(=O)N1C[C@@H](C#N)C[C@@H](N(C(=O)NCc2ccc(Cl)cc2F)C2CC2)C1]

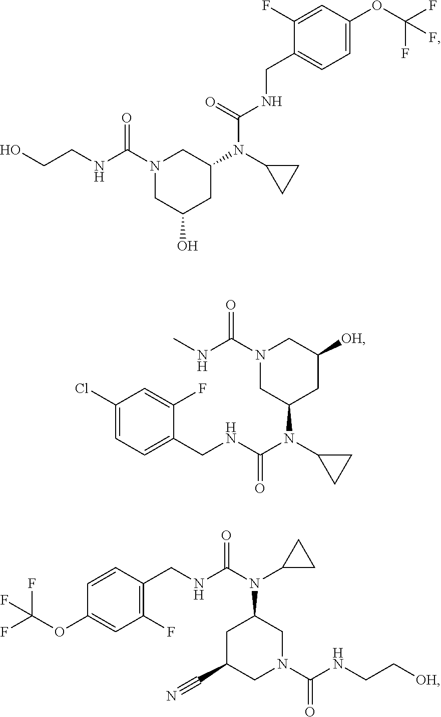 [SMILES: O=C(NCCO)N1C[C@@H](O)C[C@@H](N(C(=O)NCc2ccc(OC(F)(F)F)cc2F)C2CC2)C1 ; CNC(=O)N1C[C@@H](O)C[C@@H](N(C(=O)NCc2ccc(Cl)cc2F)C2CC2)C1 ; N#C[C@H]1C[C@@H](N(C(=O)NCc2ccc(OC(F)(F)F)cc2F)C2CC2)CN(C(=O)NCCO)C1]

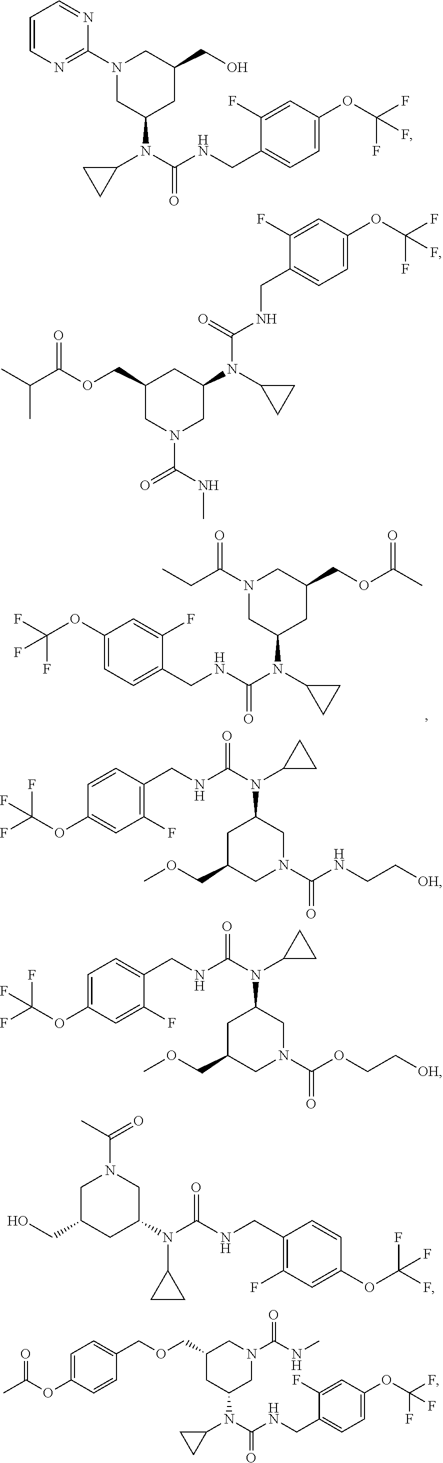

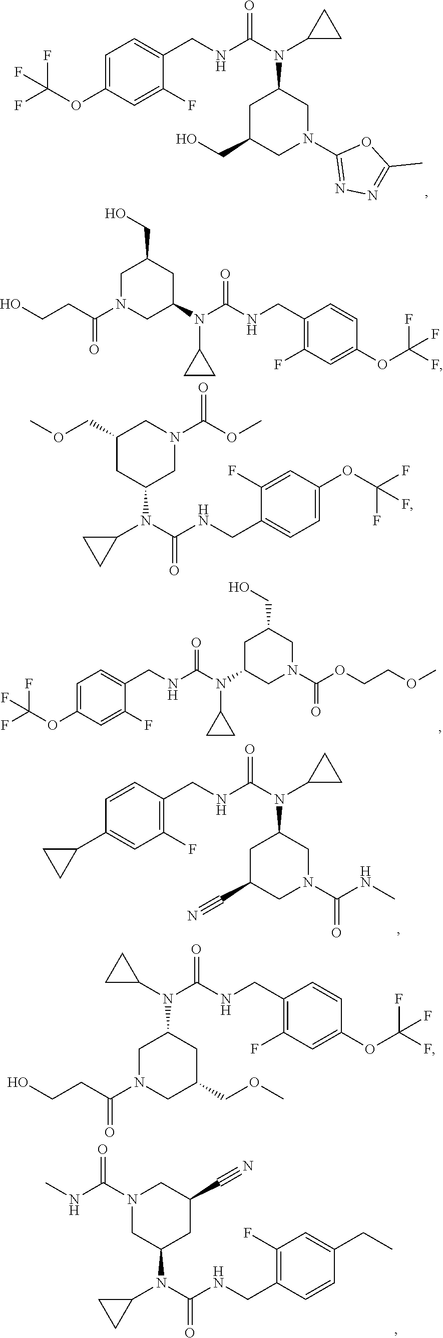

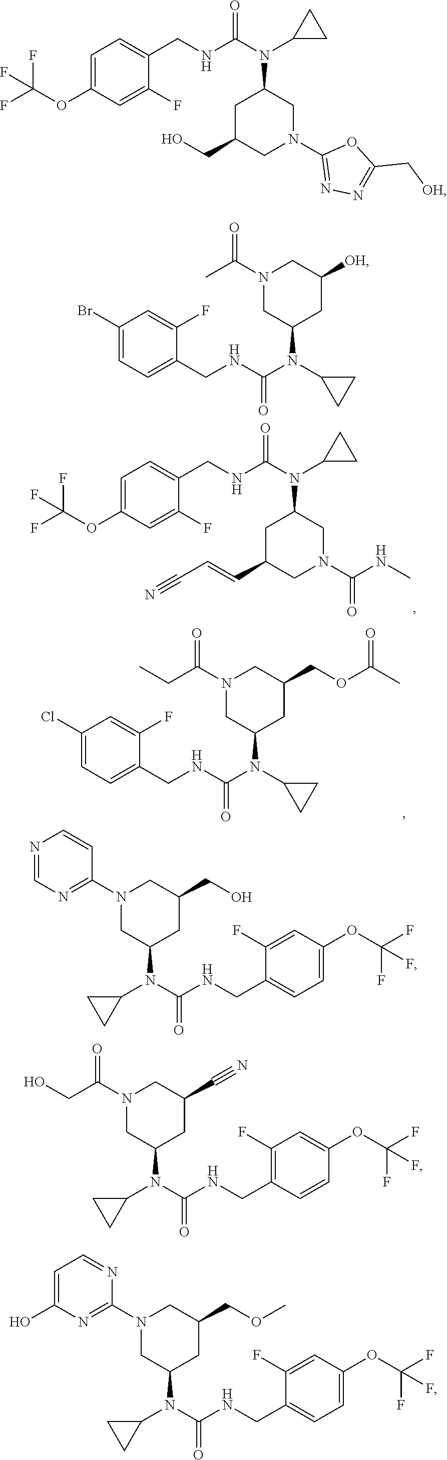

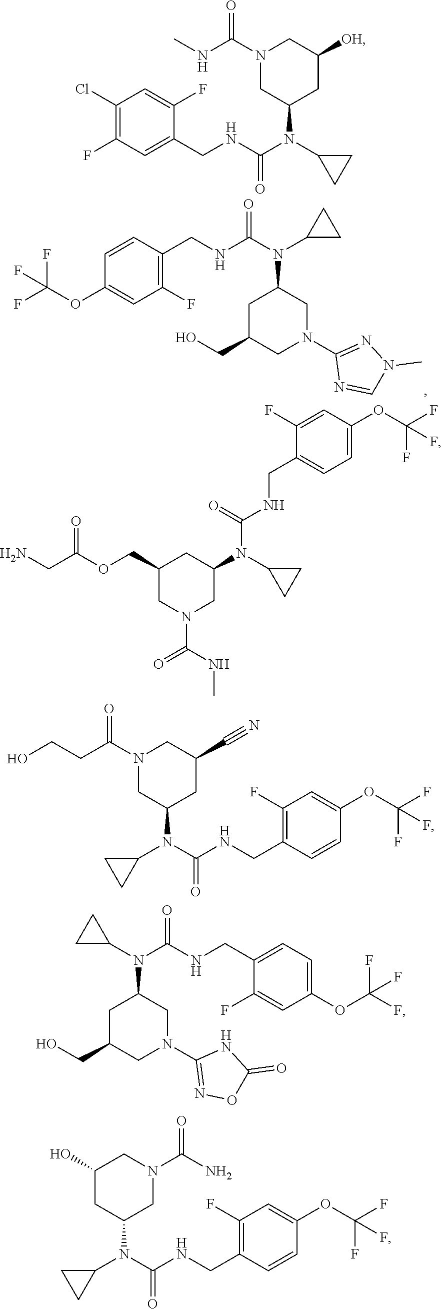

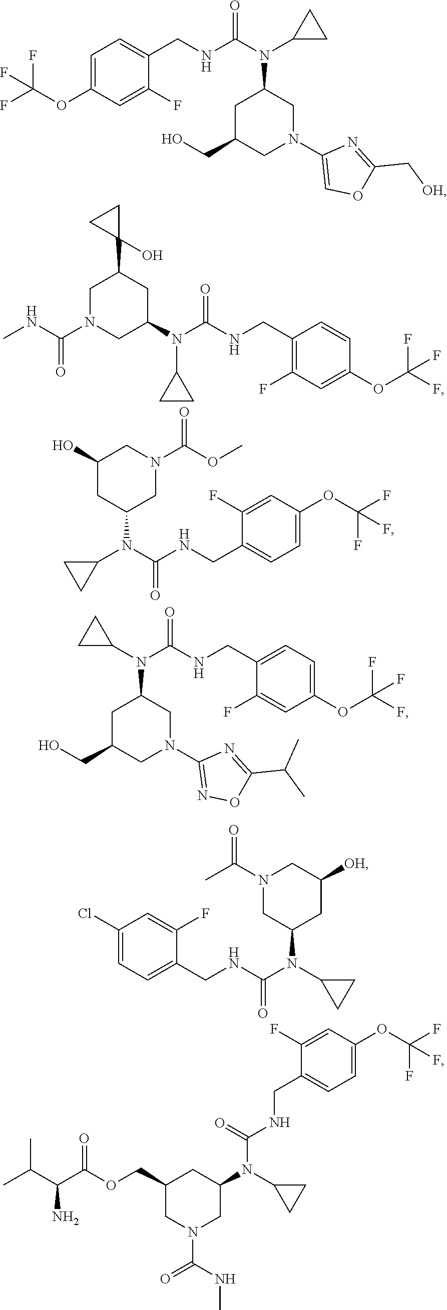

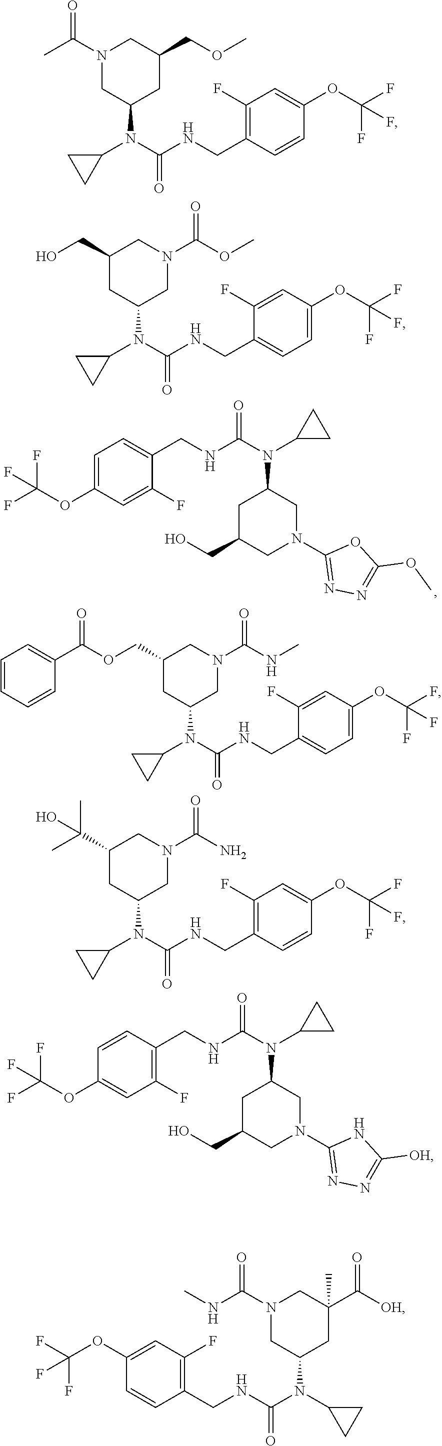

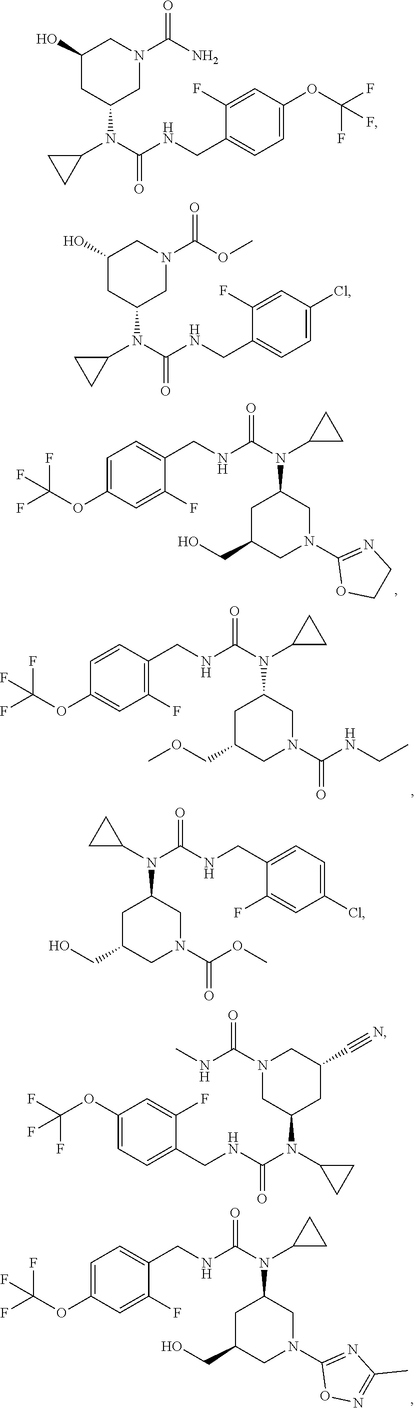

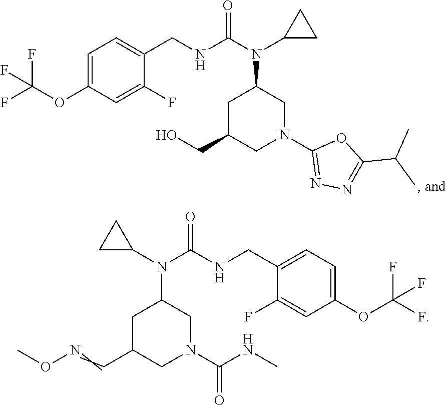

113. A compound of Formula (III): 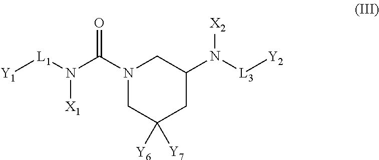
wherein:
L₁ is absent or selected from -alkyl-, -hydroxyalkyl-, -cycloalkyl-, and -heteroaryl-CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl; and
Y₆ and Y₇ together with the carbon to which they are bonded form a 4-, 5-, or 6-membered cycloalkyl or heterocyclyl;
or a pharmaceutically acceptable salt thereof.

114. A compound of Formula (IV): 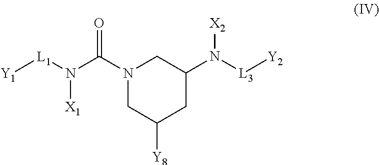
wherein:
L₁ is absent or selected from -alkyl-, -hydroxyalkyl-, -cycloalkyl-, -heteroaryl-, and -heteroaryl-CH₂—;
L₂ is absent or —CH₂—;
L₃ is absent or —C(O)—;
X₁ and X₂ are independently selected from —H, alkyl, haloalkyl, cycloalkyl, alkyl-cycloalkyl, and heterocyclyl; provided that X₁ and X₂ are not both —H;
Y₁ is selected from aryl and heteroaryl;
Y₂ is selected from alkyl, alkenyl, alkynyl, alkoxy, alkoxyalkyl, hydroxyalkyl, haloalkyl, cycloalkyl, heterocyclyl, aryl, heteroaryl, —NH(Y₂′), and —N(Y₂″)₂;
Y₂′ is selected from —H, —OH, alkyl, alkoxy, alkoxyalkyl, hydroxyalkyl, and cycloalkyl;
each Y₂″ is independently alkyl, or both instances taken together with the nitrogen atom to which they are bonded form a 5- or 6-membered heterocyclyl;
Y₈ is selected from cyano, cycloalkyl, heteroaryl, heterocyclyl, alkyl-Y₈′, and a squaramide moiety;
Y₈′ is selected from —CN, —OH, —NH₂, —NH(Y₈″), —C(O)N(Y₈′″)₂, —SO₂N(Y₈′″)₂, and a squaramide moiety;
Y₈″ is selected from alkyl, —C(O)-alkyl, and —SO₂-alkyl; and
Y₈′″ is independently for each occurrence selected from —H and alkyl;
or a pharmaceutically acceptable salt thereof.

115. A compound or a pharmaceutically acceptable salt thereof having the structure of any one of the following compounds:

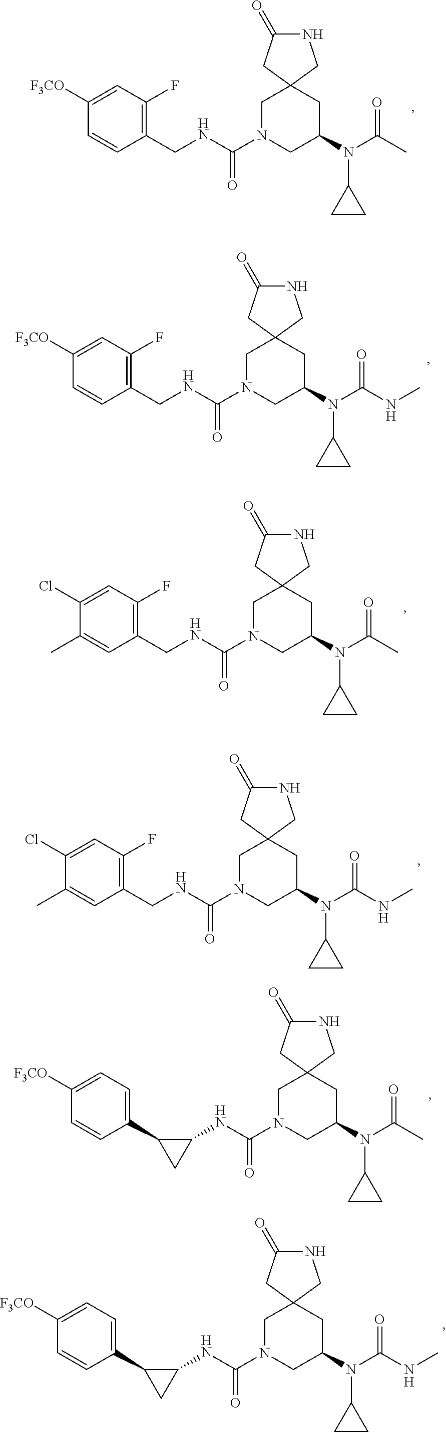

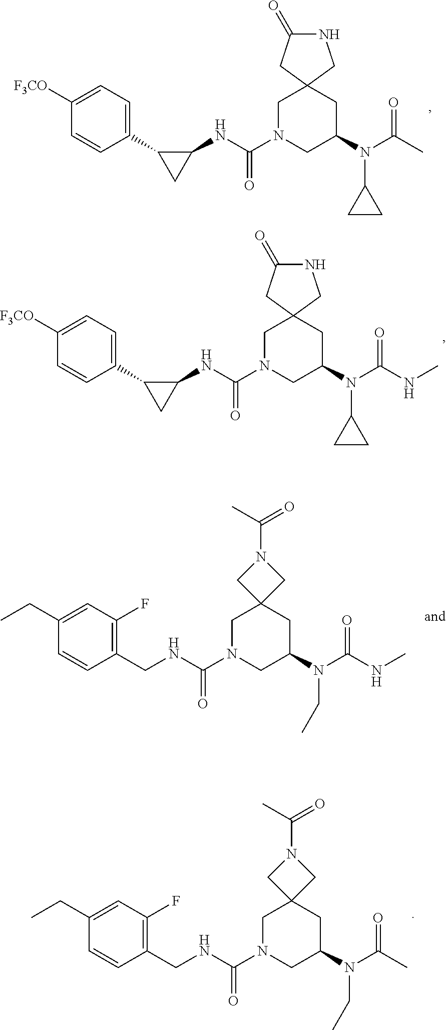

116. A compound or a pharmaceutically acceptable salt thereof having the structure of any one of the following compounds:

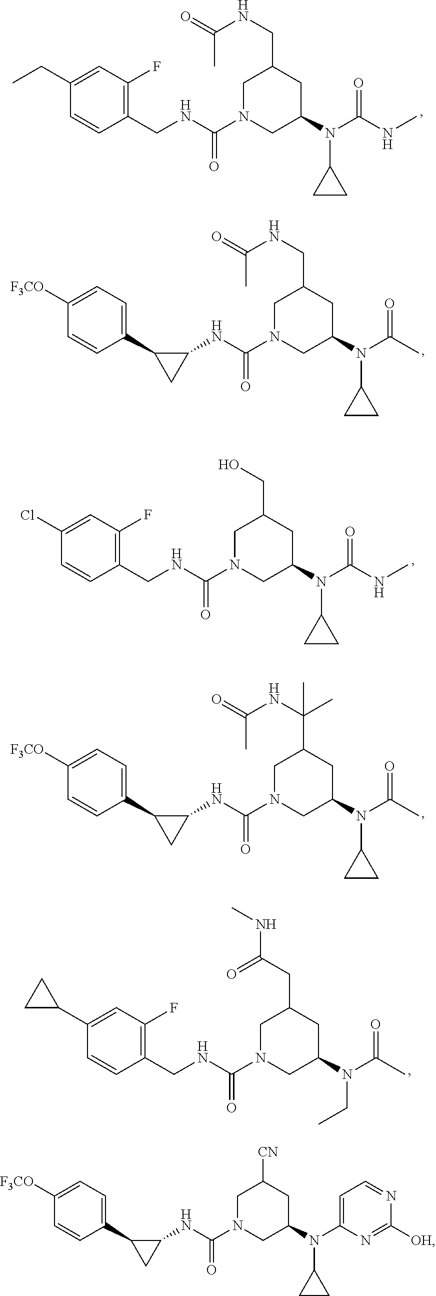

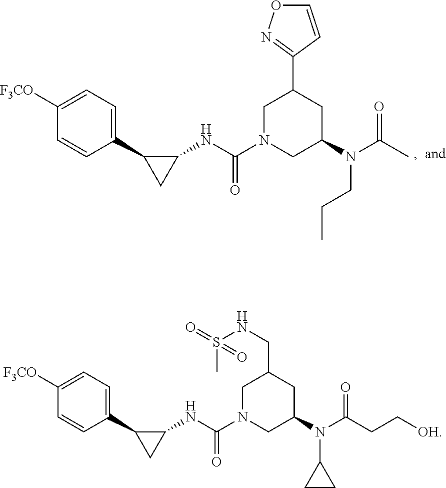 [SMILES: CCCN(C(C)=O)[C@@H]1CC(c2ccon2)CN(C(=O)N[C@@H]2C[C@H]2c2ccc(OC(F)(F)F)cc2)C1 ; CS(=O)(=O)NCC1C[C@@H](N(C(=O)CCO)C2CC2)CN(C(=O)N[C@@H]2C[C@H]2c2ccc(OC(F)(F)F)cc2)C1]

117. A pharmaceutical composition, comprising a compound of any one of claims 1-116;
and a pharmaceutical acceptable excipient.

118. A method of treating or preventing a disease or disorder associated with a genetic defect in phenylalanine hydroxylase, comprising administering to a subject in need thereof an effective amount of a compound of any one of claims 1-116.

119. A method of treating or preventing phenylketonuria, comprising administering to a subject in need thereof an effective amount of a compound of any one of claims 1-116.

120. A method of treating or preventing hyperphenylalaninemia, comprising administering to a subject in need thereof an effective amount of a compound of any one of claims 1-116.

121. The method of any one of claims 118-120, wherein the compound reduces systemic phenylalanine levels in the subject.

122. A method of treating or preventing tyrosinemia (Type I, II, or III), comprising administering to a subject in need thereof an effective amount of a compound of any one of claims 1-116.

123. The method of claim 122, wherein the compound reduces systemic tyrosine levels in the subject.

124. A method of treating or preventing nonketotic hyperglycinemia, comprising administering to a subject in need thereof an effective amount of a compound of any one of claims 1-116.

125. The method of claim 124, wherein the compound reduces systemic glycine levels in the subject.

126. A method of treating or preventing isovaleric acidemia, methylmalonic acidemia, propionic acidemia, maple syrup urine disease, DNAJC12 deficiency, urea cycle disorders, or hyperammonemia, comprising administering to a subject in need thereof an effective amount of a compound of any one of claims 1-116.

127. A method of treating or preventing diabetes, chronic kidney disease, nonalcoholic fatty liver disease, nonalcoholic steatohepatitis, metabolic syndrome, obesity related disorders, or neurodevelopmental and autism-spectrum disorders, comprising administering to a subject in need thereof an effective amount of a compound of any one of claims 1-116.

128. The method of any one of claims 118-127, wherein the compound inhibits SLC6A19 in the subject.
